(19) United States
(12) Patent Application Publication
(10) Pub. No.: US 2022/0251084 A1
(43) Pub. Date: Aug. 11, 2022

(54) PROGRANULIN MODULATORS AND METHODS OF USING THE SAME

(71) Applicant: ARKUDA THERAPEUTICS, Watertown, MA (US)

(72) Inventors: Duane A. Burnett, Cambridge, MA (US); William J. Greenlee, Teaneck, NJ (US); James C. Lanter, Cambridge, MA (US)

(21) Appl. No.: 17/614,666

(22) PCT Filed: Jun. 12, 2020
(86) PCT No.: PCT/US20/37358

§ 371 Date: Nov. 29, 2021

Related U.S. Application Data
(60) Provisional application No. 62/860,394, filed on Jun. 12, 2019.

(51) Int. Cl. C07D 471/08; C07D 401/06; C07D 401/12; C07D 401/14; C07D 413/06; C07D 413/12; C07D 417/04; C07D 417/06; C07D 417/14; C07D 453/02; C07D 471/10; C07D 487/04; C07D 487/08; C07D 491/048; C07D 498/08; C07D 498/10; C07D 519/00

(52) U.S. Cl. CPC C07D 471/08; C07D 519/00; C07D 401/12; C07D 401/14; C07D 413/06; C07D 413/12; C07D 417/04; C07D 417/06; C07D 417/14; C07D 453/02; C07D 471/10; C07D 487/04; C07D 487/08; C07D 491/048; C07D 498/08; C07D 498/10; C07D 401/06

(57) ABSTRACT

Provided herein are compounds that modulate progranulin and methods of using the compounds in progranulin-associated disorders, such as Frontotemporal dementia (FTD).

### BACKGROUND

Provided herein are compounds that modulate progranulin levels and can be useful as therapeutics for granulin (GRN)- and/or progranulin (PGRN)-associated disorders. Mutations in the GRN gene cause Frontotemporal lobar degeneration (FTLD) (see, e.g., Cruts et al., Granulin Mutations Associated with Frontotemporal Lobar Degeneration and Related Disorders: An Update, Hu Mutation, 2008 and Baker et al., Nature, 2006.) FTLD-associated mutations in GRN result in a reduction of progranulin protein expression, which suggests that haploinsufficiency of progranulin is the critical pathogenic factor in FTLD-GRN. Plasma and CSF progranulin levels are reduced by up to 70% in pathogenic GRN mutation carriers (Ghidoni, et al., Neurodegen Dis, 2012). More than 60 non-sense mutations in the GRN gene have been described. Plasma can be easily monitored for PGRN (see e.g., Meeter, Nature Neurology, volume 13, 2017). Thus, granulin- and/or progranulin-associated disorders can be modulated by compounds which increase progranulin secretion and/or activity.

All known FTLD-GRN-associated mutations cause haploinsufficiency of progranulin, suggesting that restoration of proper progranulin levels or progranulin protein function will be therapeutically beneficial for FTLD-GRN patients. Several studies have shown that even subtle reductions in progranulin levels by genetic modifiers (e.g., TMEM106B, SLPI, Rs5848) have significant effects on the age-of-onset of FTLD, increase the risk of developing FTLD, or worsen the course of autoimmune diseases such as osteoarthritis (see, e.g., Nicholson et al., J Neurochem, 2013; Cruchaga et al., Arch Neurol, 2012; and Wei et al, Plos One, 2014). Polymorphisms that affect progranulin levels have also been identified as genetic modifiers of several other neurodegenerative diseases, such as Alzheimer's disease and C9orf72-linked FTLD (see, e.g., Sheng et al., Gene, 2014 and van Blitterswijk et al., Mol Neurodegen, 2014). As such, it is contemplated herein that progranulin-targeted therapeutics are effective across multiple neurodegenerative and autoimmune disorders.

Granulins are a family of secreted and glycosylated proteins. They are cleaved from a common precursor protein called progranulin (PGRN). Progranulin is a secreted glycoprotein and is expressed in neurons, neuroglia, chondrocytes, epithelial cells and leukocytes (Toh H et al. J Mol Neurosci 2011 November; 45(3):538-48). It is a precursor protein with an N-terminal signal peptide and seven granulin motifs. Each of these granulin motifs contains 12 cysteines, which are responsible for 6 disulfide bridges in every granulin (Bateman A et al. Bioessays 2009:1245-54). Progranulin is coded by the GRN gene. Mutations in the GRN gene have been implicated in up to 25% of frontotemporal lobar degeneration, inherited in an autosomal dominant fashion with high penetrance (see, e.g., Mackenzie, Acta Neuropathologica, 114(1): 49-54 (2007)). Thus, modulation of progranulin activity is an attractive target for treating disorders associated with GRN activity or GRN-gene mutations.

### SUMMARY

Provided herein are compounds and methods for modulating progranulin. More particularly, provided are modulators of progranulin and the uses of such modulators in treating progranulin-associated disorders, e.g., Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), lysosomal storage diseases, diseases associated with inclusions and/or misfunction of C9orf72, TDP-43, FUS, UBQLN2, VCP, CHMP28, and/or MAPT, acute neurological disorders, glioblastoma, or neuroblastoma.

In one aspect, the disclosure provides compounds of Formula (I):

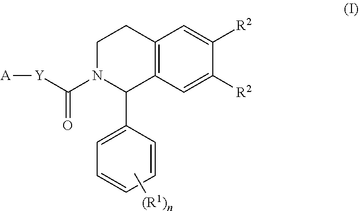

wherein A is a 4-10 membered heterocycle comprising 1 to 3 ring heteroatoms selected from N, O, and S, optionally substituted with 1 to 3 R³;

Y is C₀₋₆alkylene, C₀₋₆alkylene-O—C₀₋₆alkylene, C₀₋₆alkylene-NR^{N}, C₀₋₆alkylene-SO₂, CO, CO₂, or CONH, wherein C₀₋₆alkylene is optionally substituted with 1 or 2 R⁴;

each R¹ is independently halo, —O—CH₂—C₆aryl-(OCH₂CH₂)ₚ—OR⁵, or —O—CH₂—Het-(OCH₃), wherein Het is a 6-membered heteroaryl comprising 2 ring N atoms;

one R² is H and the other is H, CN, COOC₁₋₆alkyl, CONHC₁₋₆alkyl, SO₂CH₃, or O-propargyl;

R³ is C₁₋₆alkyl, halo, C₀₋₆alkylene-OH, C₀₋₆alkylene-O-propargyl, propargyl, or C₀₋₆alkylene-NR^{N}R^{N};

each R⁴ is independently F, OH, or OC₁₋₆alkyl, or two R⁴ together with the carbon atom to which they are attached form cyclopropyl;

R⁵ is C₁₋₆alkyl or propargyl;

each R^{N} is independently H or C₁₋₆alkyl;

n is 1-3; and

p is 0-2;

with the proviso that

(a) if A comprises

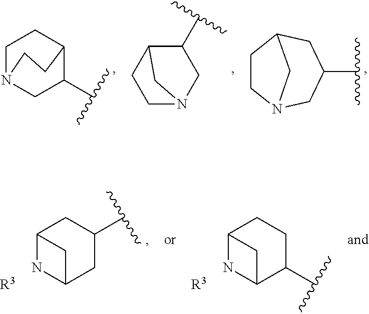

Y is CH₂, O, or NR^{N}, then one R² is H and the other R² is not H; or

(b) if Y is CH₂, O, or NR^{N} and each R² is H, then A does not comprise

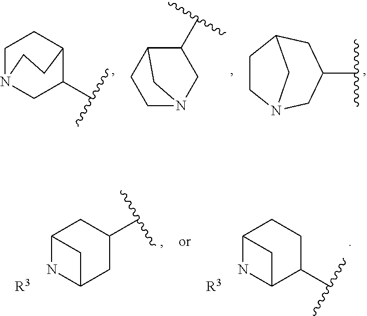

In some embodiments, A is a 4-10 membered heterocycle comprising 1 to 3 ring heteroatoms selected from N, O, and S, optionally substituted with 1 or 2 R³;

Y is C₀₋₆alkylene, C₀₋₆alkylene-O—C₀₋₆alkylene, C₀₋₆alkylene-NR^{N}, C₀₋₆alkylene-SO₂, CO₂—, or CONH—, wherein C₀₋₆alkylene is optionally substituted with 1 or 2 R⁴;

each R¹ is independently halo, —O—CH₂—C₆aryl-(OCH₂CH₂)ₚ—OR⁵, or —O—CH₂—Het-(OCH₃), wherein Het is a 6-membered heteroaryl comprising 2 ring N atoms;

one R² is H and the other is H, CN, COOC₁₋₆alkyl, CONHC₁₋₆alkyl, SO₂CH₃, or O-propargyl;

R³ is C₁₋₆alkyl;

each R⁴ is independently F or OR³;

R⁵ is C₁₋₆alkyl or propargyl;

R^{N} is H or C₁₋₆alkyl;

n is 1-3; and

p is 0-2;

with the proviso that

(a) if A comprises

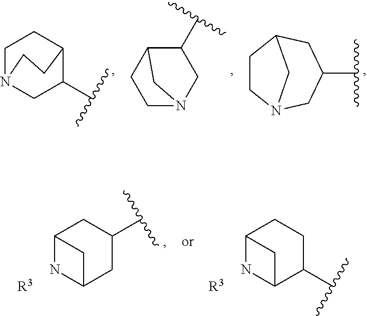

and Y is CH₂, O, or NR^{N}, then one R² is H and the other R² is not H; or

(b) if Y is CH₂, O, or NR^{N} and each R² is H, then A does not comprise,

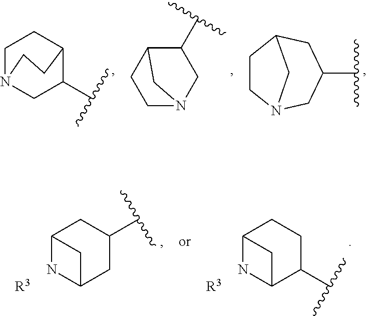

In some cases, the compounds are compounds of Formula (Ia) or (Ib):

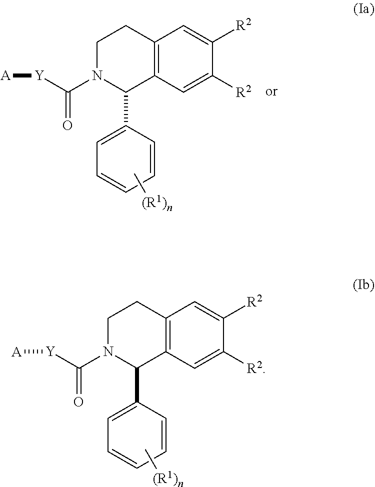

In some cases, the compounds are compounds of Formula (Ic) or (Id):

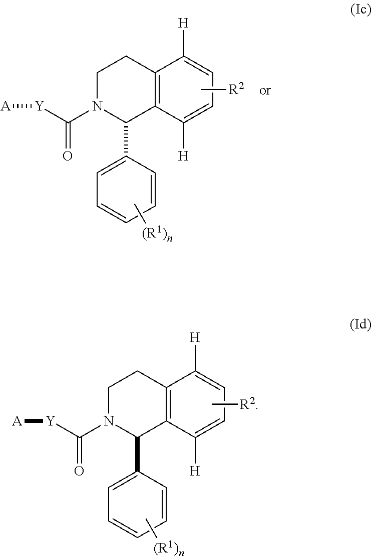

Further provided are methods of modulating progranulin in a subject. In some embodiments, provided are methods of treating a progranulin-associated disorder in a subject.

Other aspects of the disclosure include a compound as disclosed herein for use in the preparation of a medicament for the modulation of progranulin, and the use of a compound as disclosed herein in a method of treating or preventing a progranulin-associated disorder in a subject.

### DETAILED DESCRIPTION

### Compounds as Progranulin Modulators

Provided herein are compounds that can modulate progranulin production and/or secretion.

The disclosure provides compounds of Formula (I):

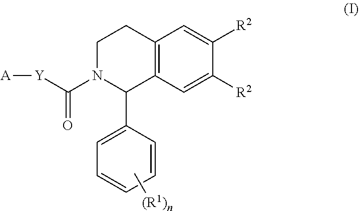

wherein A is a 4-10 membered heterocycle comprising 1 to 3 ring heteroatoms selected from N, O, and S, optionally substituted with 1 to 3 R³;

Y is C₀₋₆alkylene, C₀₋₆alkylene-O—C₀₋₆alkylene, C₀₋₆alkylene-NR^{N}, C₀₋₆alkylene-SO₂, CO, CO₂, or CONH, wherein C₀₋₆alkylene is optionally substituted with 1 or 2 R⁴;

each R¹ is independently halo, —O—CH₂—C₆aryl-(OCH₂CH₂)ₚ—OR⁵, or —O—CH₂—Het-(OCH₃), wherein Het is a 6-membered heteroaryl comprising 2 ring N atoms;

one R² is H and the other is H, CN, COOC₁₋₆alkyl, CONHC₁₋₆alkyl, SO₂CH₃, or O-propargyl;

R³ is C₁₋₆alkyl, halo, C₀₋₆alkylene-OH, C₀₋₆alkylene-O-propargyl, propargyl, or C₀₋₆alkylene-NR^{N}R^{N};

each R⁴ is independently F, OH, or OC₁₋₆alkyl, or two R⁴ together with the carbon atom to which they are attached form cyclopropyl;

R⁵ is C₁₋₆alkyl or propargyl;

each R^{N} is independently H or C₁₋₆alkyl;

n is 1-3; and

p is 0-2;

with the proviso that

(a) if A comprises

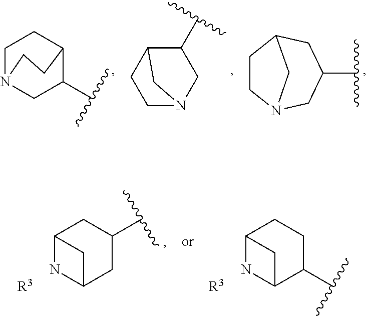

and Y is CH₂, O, or NR^{N}, then one R² is H and the other R² is not H; or

(b) if Y is CH₂, O, or NR^{N} and each R² is H, then A does not comprise

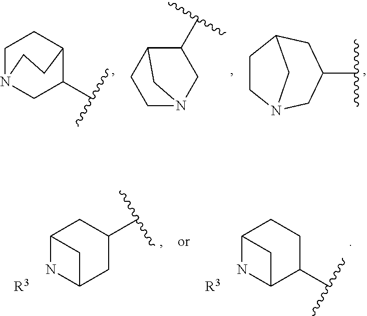

In some embodiments, A is a 4-10 membered heterocycle comprising 1 to 3 ring heteroatoms selected from N, O, and S, optionally substituted with 1 or 2 R³;

Y is C₀₋₆alkylene, C₀₋₆alkylene-O—C₀₋₆alkylene, C₀₋₆alkylene-NR^{N}, C₀₋₆alkylene-SO₂, CO₂—, or CONH—, wherein C₀₋₆alkylene is optionally substituted with 1 or 2 R⁴;

each R¹ is independently halo, —O—CH₂—C₆aryl-(OCH₂CH₂)ₚ—OR⁵, or —O—CH₂—Het-(OCH₃), wherein Het is a 6-membered heteroaryl comprising 2 ring N atoms;

one R² is H and the other is H, CN, COOC₁₋₆alkyl, CONHC₁₋₆alkyl, SO₂CH₃, or O-propargyl;

R³ is C₁₋₆alkyl;

each R⁴ is independently F or OR³;

R⁵ is C₁₋₆alkyl or propargyl;

R^{N} is H or C₁₋₆alkyl;

n is 1-3; and

p is 0-2;

with the proviso that

(a) if A comprises

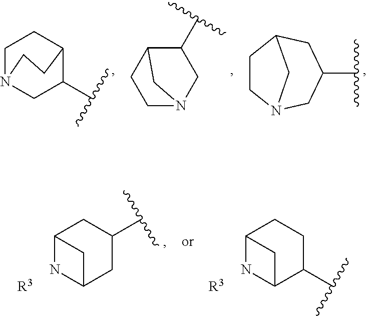

and Y is CH₂, O, or NR^{N}, then one R² is H and the other R² is not H; or

(b) if Y is CH₂, O, or NR^{N} and each R² is H, then A does not comprise

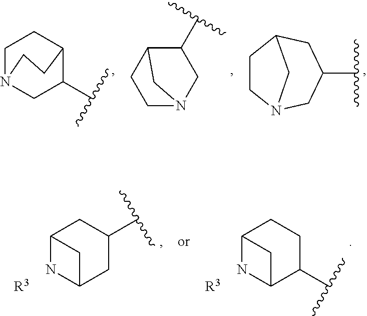

In some embodiments, A is a 4-10 membered heterocycle comprising 1 to 3 ring heteroatoms selected from N, O, and S, optionally substituted with 1 or 2 R³;

Y is C₀₋₆alkylene, C₀₋₆alkylene-O, C₀₋₆alkylene-O—C₀₋₆alkylene, C₀₋₆alkylene-NR³, C₀₋₆alkylene-SO₂, CO₂—, or CONH—, wherein C₀₋₆alkylene is optionally substituted with 1 or 2 R⁴;

each R¹ is independently halo, —O—CH₂—C₆aryl-(OCH₂CH₂)ₚ—OR⁵, or —O—CH₂—Het-(OCH₃), wherein Het is a 6-membered heteroaryl comprising 2 ring N atoms;

one R² is H and the other is H, CN, COOC₁₋₆alkyl, CONHC₁₋₆alkyl, SO₂CH₃, or O-propargyl;

R³ is H or C₁₋₆alkyl;

each R⁴ is independently F or OR³;

R⁵ is C₁₋₆alkyl or propargyl;

n is 1-3; and

p is 0-2;

with the proviso that

(a) if A comprises

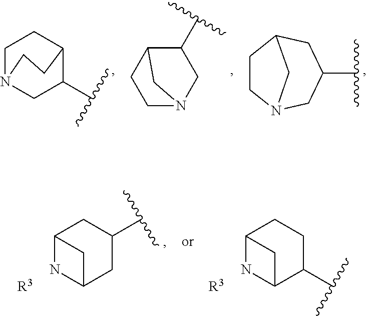

and Y is CH₂, O, or NR³, then one R² is H and the other R² is not H; or

(b) if Y is CH₂, O, or NR³ and each R² is H, then A does not comprise

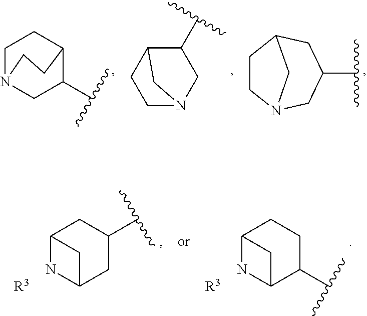

In some embodiments, the compound of Formula (I) has the structure of Formula (Ia) or (Ib):

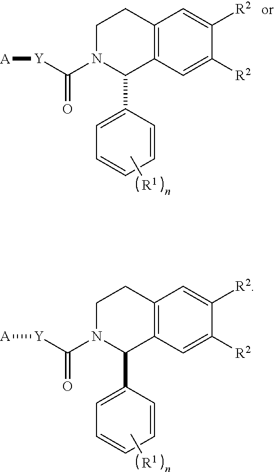

In some cases, the compound of Formula (I) has the structure of Formula (Ia). In some cases, the compound of Formula (I) has the structure of Formula (Ib).

In some embodiments, the compound of Formula (I) has the structure of Formula (Ic) or (Id):

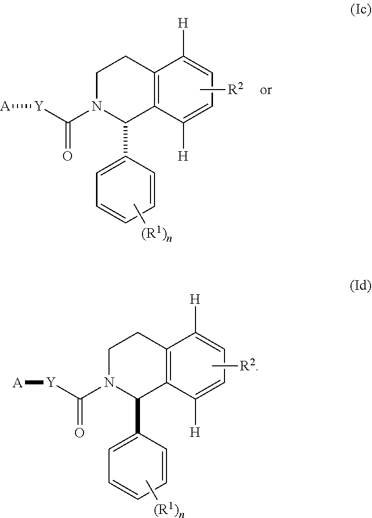

In some cases, the compound of Formula (I) has the structure of Formula (Ic). In some cases, the compound of Formula (I) has the structure of Formula (Id).

In some cases, A comprises a 4 membered heterocycle. In some cases, A comprises a 5 membered heterocycle. In some cases, A comprises a 6 membered heterocycle. In some cases, A comprises a 7 membered heterocycle. In some cases, A comprises an 8 membered heterocycle. In some cases, A comprises a 9 membered heterocycle. In some cases, A comprises a 10 membered heterocycle. In some cases, A comprises a 4-, 6-, 8-, or 10-membered heterocycle comprising 1 or 2 ring heteroatoms selected from N and O. In some cases, A comprises an 8-membered heterocycle comprising 1 or 2 ring heteroatoms selected from N and O. In some cases, A is substituted with 1 to 3 R³. In some cases, A is substituted with 1 or 2 R³. In some cases, A is unsubstituted.

In some cases, A comprises

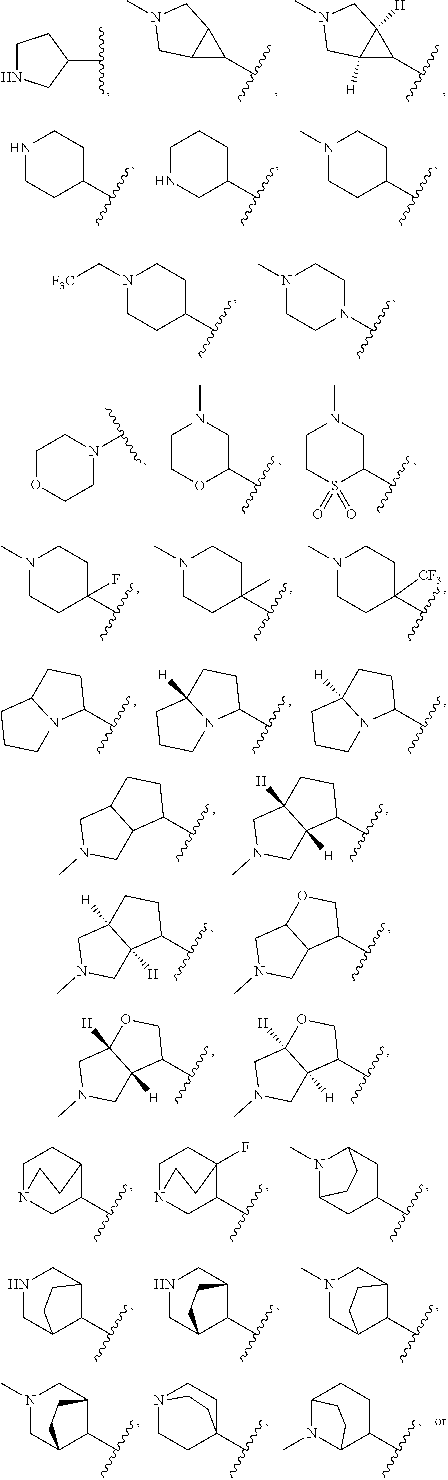

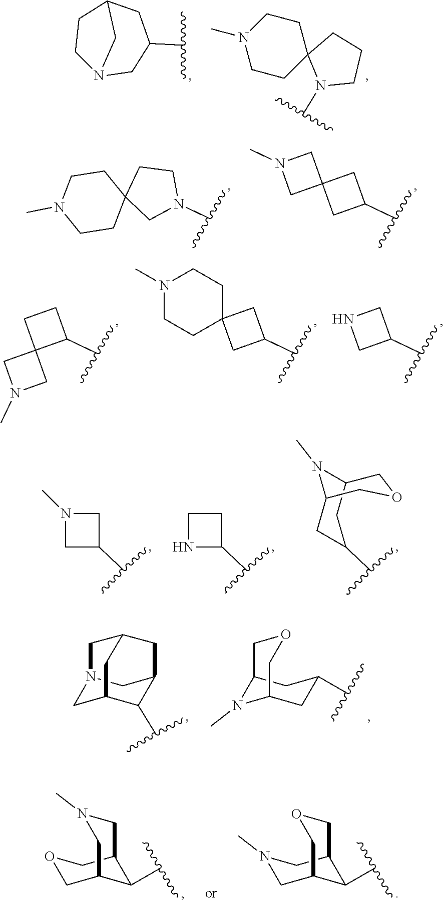

In some cases, A comprises

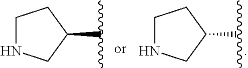 [SMILES: *[C@@H]1CCNC1 ; *[C@H]1CCNC1]

In some cases, A comprises

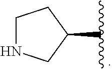 [SMILES: *[C@@H]1CCNC1]

In some cases, A comprises

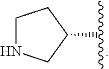 [SMILES: *[C@H]1CCNC1]

In some cases, A comprises

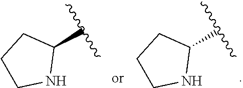 [SMILES: *[C@@H]1CCCN1 ; *[C@H]1CCCN1]

In some cases, A comprises

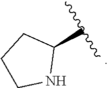 [SMILES: *[C@@H]1CCCN1]

In some cases, A comprises

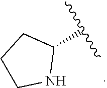 [SMILES: *[C@H]1CCCN1]

In some cases, A comprises

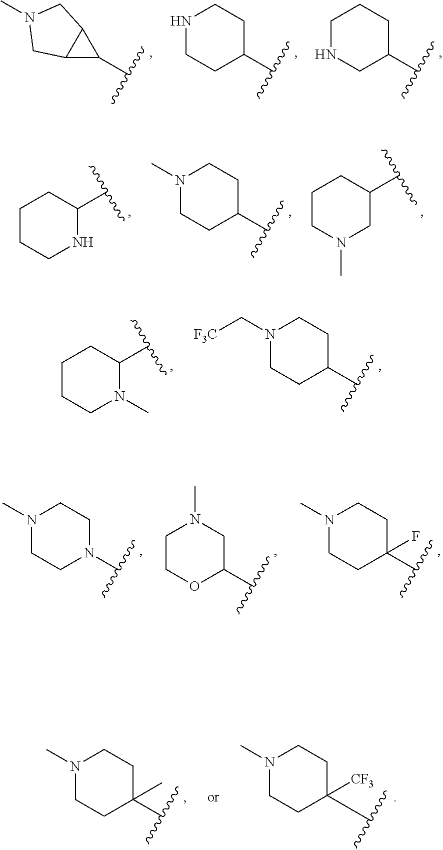

In some cases, A comprises

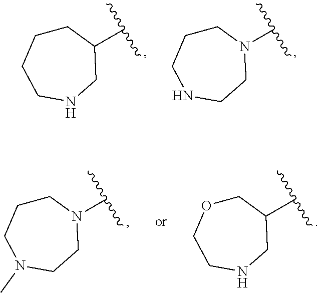

In some cases, A comprises

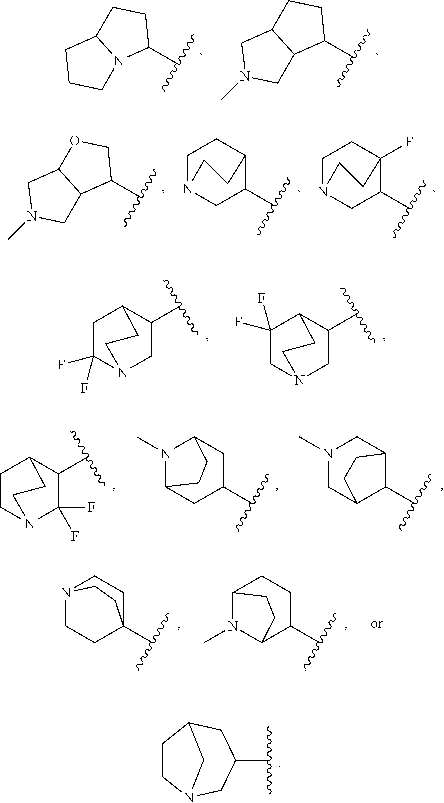

In some cases, A comprises

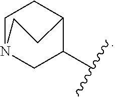 [SMILES: *C1CN2CCC1CC2]

In some cases, A comprises

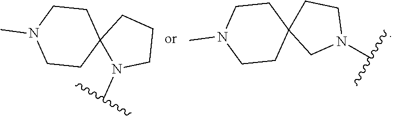

In some cases, A comprises

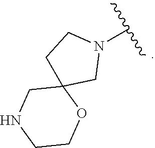 [SMILES: *N1CCC2(CNCCO2)C1]

In some cases, A comprises

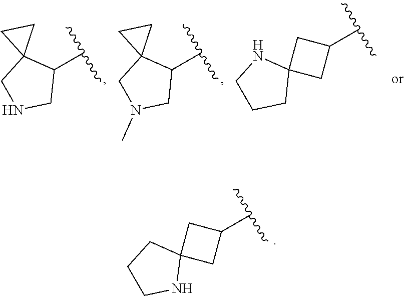

In some cases, A comprises

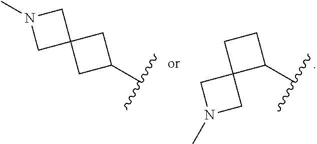

In some cases, A comprises

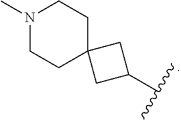 [SMILES: *C1CC2(CCN(C)CC2)C1]

In some cases, Y is C₀₋₆alkylene, C₀₋₆alkylene-O—C₀₋₆alkylene (e.g., C₀₋₆alkylene-O), C₀₋₆alkylene-NR^{N}, C₀₋₆alkylene-SO₂, CO, CO₂—, or CONH—. In some cases, Y is C₀₋₆alkylene, C₀₋₆alkylene-O—C₀₋₆alkylene (e.g., C₀₋₆alkylene-O), C₀₋₆alkylene-NR^{N}, C₀₋₆alkylene-SO₂, CO₂—, or CONH—. In some cases, Y is C₀ alkylene (i.e., a bond). In some cases, Y is C₁₋₆alkylene, C₁₋₆alkylene-O, or C₁₋₆alkylene-NR^{N}.

In some cases, Y is C₀₋₆alkylene-O or C₀₋₆alkylene-NR^{N}. In some cases, Y is C₀₋₆alkylene-O. In some cases, Y is C₁₋₆alkylene-O. In some cases, Y is C₁₋₆alkylene-NR^{N}. In some cases, Y is NH or 0. In some cases, Y is NH. In some cases, Y is 0.

In some cases, the C₁₋₆alkylene of a Y moiety is unsubstituted. In some cases, C₁₋₆ alkylene is substituted with 1 or 2 R⁴. In some cases, C₁₋₆alkylene is substituted with 1 R⁴.

In some cases, each R¹ is independently halo. In some cases, each R¹ is independently —O—CH₂—C₆aryl-(OCH₂CH₂)ₚ—OR⁵ or —O—CH₂—Het-(OCH₃), wherein Het is a 6-membered heteroaryl comprising 2 ring N atoms. In some cases, each R¹ is independently —O—CH₂—C₆aryl-(OCH₂CH₂)ₚ—OR⁵. In some cases, each R¹ is independently —O—CH₂—Het-(OCH₃).

In some cases, at least one R¹ is halo. In some cases, at least one R¹ is F. In some cases, each R¹ is F. In some cases, at least one R¹ is —O—CH₂—C₆aryl-(OCH₂CH₂)ₚ—OR⁵. In some cases, at least one R¹ is —O—CH₂—Het. In some cases, Het comprises 2-pyrimidyl or 5-pyrimidyl optionally substituted with OMe. In some cases, Het comprises 2-pyrimidyl. In some cases, Het comprises 5-pyrimidyl. In some cases, Het is unsubstituted. In some cases, Het is substituted with OMe.

In some cases, both R² are H. In some cases, one R² is H and the other is CN, COOC₁₋₆alkyl, CONHC₁₋₆alkyl, SO₂CH₃, or O-propargyl. In some cases, In some cases, one R² is H and the other is CN. In some cases, one R² is H and the other is COOCH₃ or CONHCH₃. In some cases, one R² is H and the other is COOCH₃. In some cases, one R² is H and the other is CONHCH₃. In some cases, In some cases, one R² is H and the other is SO₂CH₃. In some cases, In some cases, one R² is H and the other is O-propargyl.

In some cases, R³ is C₁₋₆alkyl, halo, C₀₋₆alkylene-OH, C₀₋₆alkylene-O-propargyl, propargyl, or C₀₋₆alkylene-NR^{N}R^{N}. In some cases, R³ is C₁₋₆alkyl. In some cases, R³ is C₁₋₆alkyl. In some cases, R³ is halo. In some cases, R³ is fluoro. In some cases, R³ is C₀₋₆alkylene-OH. In some cases, R³ is C₁alkylene-OH. In some cases, R³ is C₀alkylene-OH, i.e., R³ is OH. In some cases, R³ is C₀₋₆alkylene-O-propargyl. In some cases, R³ is C₂alkylene-O-propargyl. In some cases, R³ is propargyl. In some cases, R³ is C₀₋₆alkylene-NR^{N}R^{N}. In some cases, R³ is C₁alkylene-NR^{N}R^{N}. In some cases, R³ is C₀alkylene-NR^{N}R^{N}, i.e., R³ is NR^{N}R^{N}.

In some cases, each R⁴ is independently F, OH, or OC₁₋₆alkyl, or two R⁴ together with the carbon atom to which they are attached form cyclopropyl. In some cases, R⁴ is F, OH, or OCH₃. In some cases, R⁴ is F. In some cases, R⁴ is OH or OCH₃. In some cases, R⁴ is OH. In some cases, R⁴ is OCH₃. In some cases, two R⁴ together with the carbon atom to which they are attached form cyclopropyl.

In some cases, R⁵ is methyl. In some cases, R⁵ is propargyl.

In some cases, each R^{N} is independently H or C₁₋₆alkyl. In some cases, R^{N} is H. In some cases, R^{N} is C₁₋₆alkyl. In some cases, R^{N} is methyl.

In some cases, p is 0. In some cases, p is 1. In some cases, p is 2.

In some cases, n is 1. In some cases, n is 2. In some cases, n is 3.

Specific compounds contemplated include those listed in Table A, or a pharmaceutically acceptable salt thereof:

TABLE A

| Structure | Compound No. |
|---|---|
| 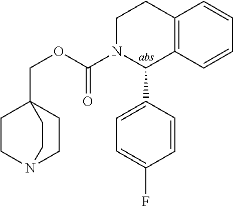 [SMILES: O=C(OCC12CCN(CC1)CC2)N1CCc2ccccc2[C@@H]1c1ccc(F)cc1] | Compound 2001 |
| 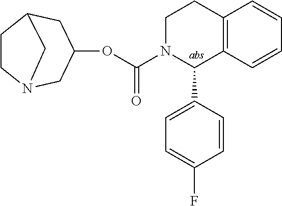 [SMILES: O=C(OC1CC2CCN(C2)C1)N1CCc2ccccc2[C@@H]1c1ccc(F)cc1] | Compound 2002 |
| 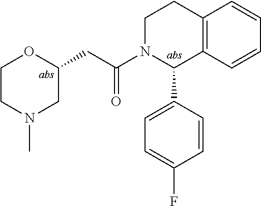 [SMILES: CN1CCO[C@H](CC(=O)N2CCc3ccccc3[C@@H]2c2ccc(F)cc2)C1] | Compound 2003 |
| 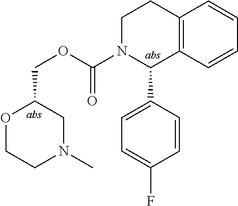 [SMILES: CN1CCO[C@H](COC(=O)N2CCc3ccccc3[C@@H]2c2ccc(F)cc2)C1] | Compound 2004 |
| 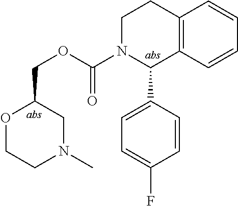 [SMILES: CN1CCO[C@@H](COC(=O)N2CCc3ccccc3[C@@H]2c2ccc(F)cc2)C1] | Compound 2005 |
| 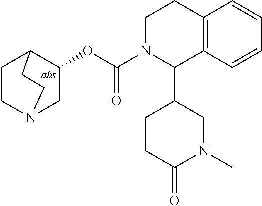 [SMILES: CN1CC(C2c3ccccc3CCN2C(=O)O[C@@H]2CN3CCC2CC3)CCC1=O] | Compound 2006 |
| 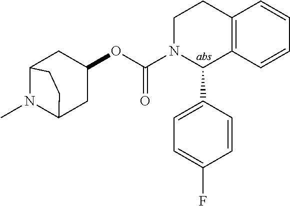 | Compound 2007 |
| 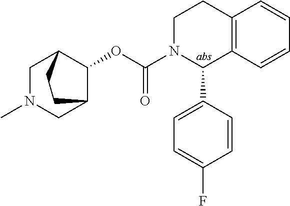 [SMILES: CN1C[C@H]2CC[C@@H](C1)[C@H]2OC(=O)N1CCc2ccccc2[C@@H]1c1ccc(F)cc1] | Compound 2008 |
| 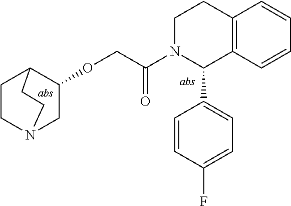 [SMILES: O=C(CO[C@@H]1CN2CCC1CC2)N1CCc2ccccc2[C@@H]1c1ccc(F)cc1] | Compound 2009 |
| 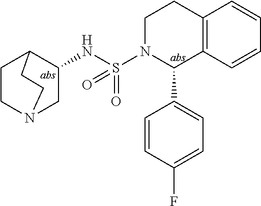 [SMILES: O=S(=O)(N[C@@H]1CN2CCC1CC2)N1CCc2ccccc2[C@@H]1c1ccc(F)cc1] | Compound 2010 |
| 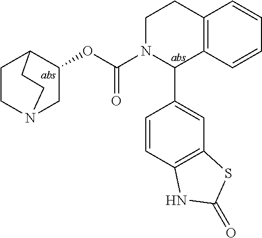 [SMILES: O=C(O[C@@H]1CN2CCC1CC2)N1CCc2ccccc2C1c1ccc2[nH]c(=O)sc2c1] | Compound 2011 |
| 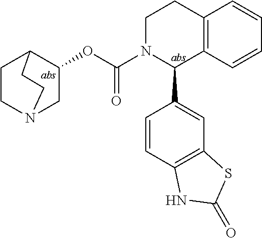 [SMILES: O=C(O[C@@H]1CN2CCC1CC2)N1CCc2ccccc2[C@H]1c1ccc2[nH]c(=O)sc2c1] | Compound 2012 |
| 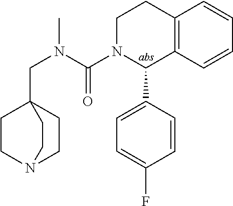 [SMILES: CN(CC12CCN(CC1)CC2)C(=O)N1CCc2ccccc2[C@@H]1c1ccc(F)cc1] | Compound 2013 |
| 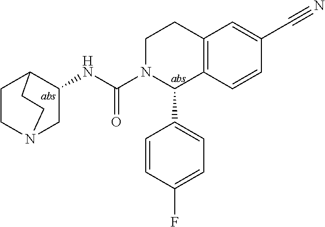 [SMILES: N#Cc1ccc2c(c1)CCN(C(=O)N[C@@H]1CN3CCC1CC3)[C@H]2c1ccc(F)cc1] | Compound 2014 |
| 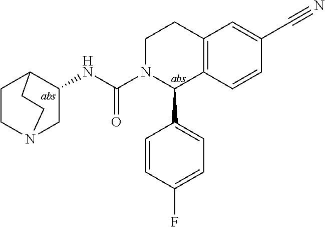 [SMILES: N#Cc1ccc2c(c1)CCN(C(=O)N[C@@H]1CN3CCC1CC3)[C@@H]2c1ccc(F)cc1] | Compound 2015 |
| 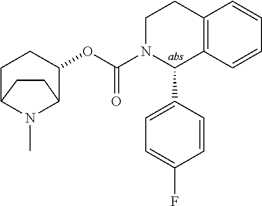 [SMILES: CN1C2CCC1[C@@H](OC(=O)N1CCc3ccccc3[C@@H]1c1ccc(F)cc1)CC2] | Compound 2016 |
| 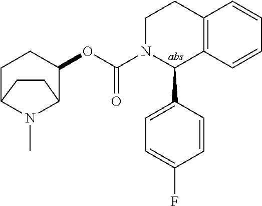 [SMILES: CN1C2CCC1[C@H](OC(=O)N1CCc3ccccc3[C@H]1c1ccc(F)cc1)CC2] | Compound 2017 |
| 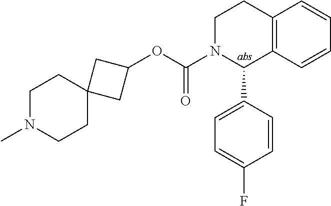 [SMILES: CN1CCC2(CC1)CC(OC(=O)N1CCc3ccccc3[C@@H]1c1ccc(F)cc1)C2] | Compound 2018 |
| 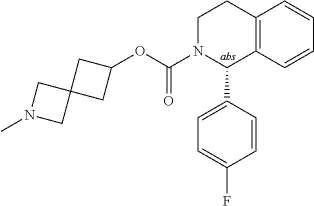 [SMILES: CN1CC2(CC(OC(=O)N3CCc4ccccc4[C@@H]3c3ccc(F)cc3)C2)C1] | Compound 2019 |
| 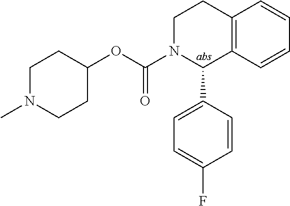 [SMILES: CN1CCC(OC(=O)N2CCc3ccccc3[C@@H]2c2ccc(F)cc2)CC1] | Compound 2020 |
| 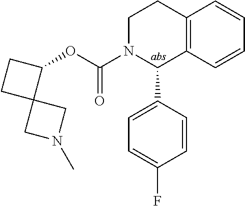 [SMILES: CN1CC2(CC[C@@H]2OC(=O)N2CCc3ccccc3[C@@H]2c2ccc(F)cc2)C1] | Compound 2021 |
| 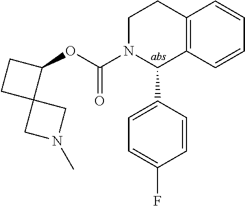 [SMILES: CN1CC2(CC[C@H]2OC(=O)N2CCc3ccccc3[C@@H]2c2ccc(F)cc2)C1] | Compound 2022 |
| 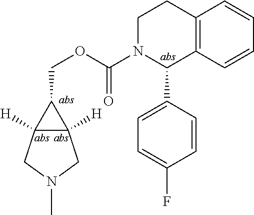 [SMILES: CN1C[C@@H]2[C@@H](COC(=O)N3CCc4ccccc4[C@@H]3c3ccc(F)cc3)[C@@H]2C1] | Compound 2023 |
| 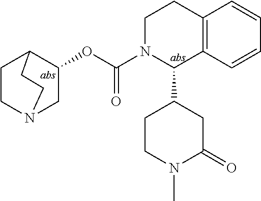 [SMILES: CN1CCC([C@H]2c3ccccc3CCN2C(=O)O[C@@H]2CN3CCC2CC3)CC1=O] | Compound 2024 |
| 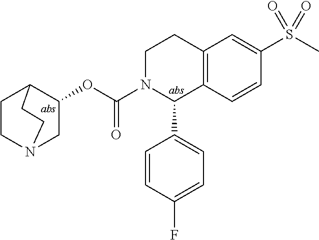 [SMILES: CS(=O)(=O)c1ccc2c(c1)CCN(C(=O)O[C@@H]1CN3CCC1CC3)[C@H]2c1ccc(F)cc1] | Compound 2025 |
| 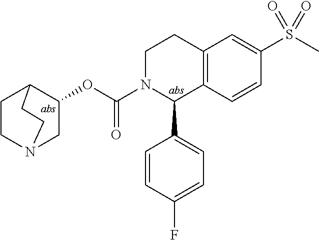 [SMILES: CS(=O)(=O)c1ccc2c(c1)CCN(C(=O)O[C@@H]1CN3CCC1CC3)[C@@H]2c1ccc(F)cc1] | Compound 2026 |
| 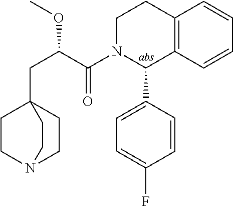 [SMILES: CO[C@@H](CC12CCN(CC1)CC2)C(=O)N1CCc2ccccc2[C@@H]1c1ccc(F)cc1] | Compound 2027 |
| 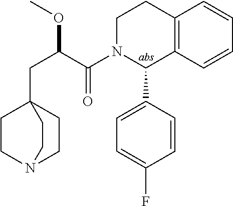 [SMILES: CO[C@H](CC12CCN(CC1)CC2)C(=O)N1CCc2ccccc2[C@@H]1c1ccc(F)cc1] | Compound 2028 |
| 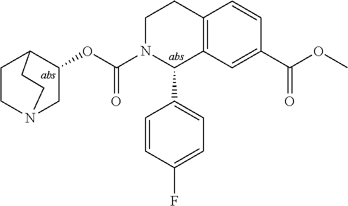 [SMILES: COC(=O)c1ccc2c(c1)[C@H](c1ccc(F)cc1)N(C(=O)O[C@@H]1CN3CCC1CC3)CC2] | Compound 2029 |
| 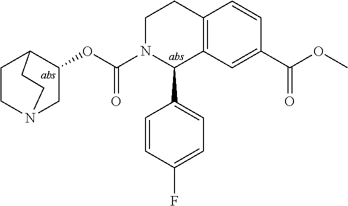 [SMILES: COC(=O)c1ccc2c(c1)[C@@H](c1ccc(F)cc1)N(C(=O)O[C@@H]1CN3CCC1CC3)CC2] | Compound 2030 |
| 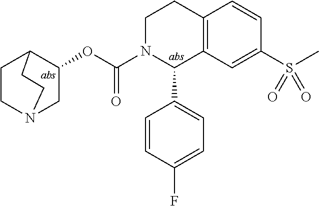 [SMILES: CS(=O)(=O)c1ccc2c(c1)[C@H](c1ccc(F)cc1)N(C(=O)O[C@@H]1CN3CCC1CC3)CC2] | Compound 2031 |
| 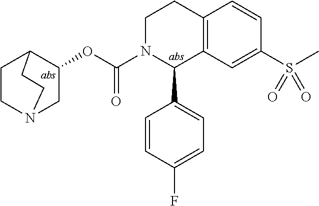 [SMILES: CS(=O)(=O)c1ccc2c(c1)[C@@H](c1ccc(F)cc1)N(C(=O)O[C@@H]1CN3CCC1CC3)CC2] | Compound 2032 |
| 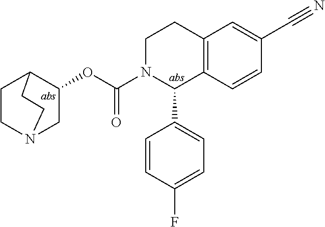 [SMILES: N#Cc1ccc2c(c1)CCN(C(=O)O[C@@H]1CN3CCC1CC3)[C@H]2c1ccc(F)cc1] | Compound 2033 |
| 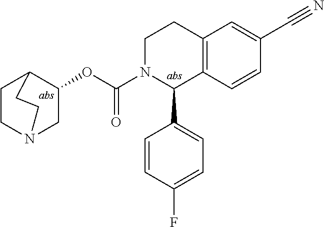 [SMILES: N#Cc1ccc2c(c1)CCN(C(=O)O[C@@H]1CN3CCC1CC3)[C@@H]2c1ccc(F)cc1] | Compound 2034 |
| 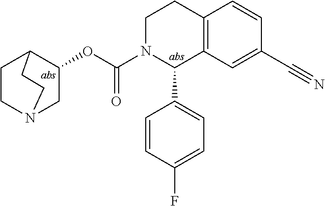 [SMILES: N#Cc1ccc2c(c1)[C@H](c1ccc(F)cc1)N(C(=O)O[C@@H]1CN3CCC1CC3)CC2] | Compound 2035 |
| 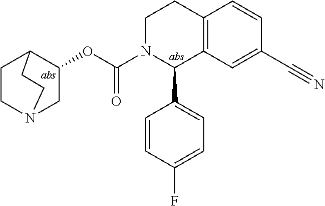 [SMILES: N#Cc1ccc2c(c1)[C@@H](c1ccc(F)cc1)N(C(=O)O[C@@H]1CN3CCC1CC3)CC2] | Compound 2036 |
| 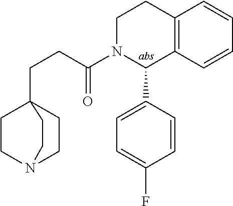 [SMILES: O=C(CCC12CCN(CC1)CC2)N1CCc2ccccc2[C@@H]1c1ccc(F)cc1] | Compound 2037 |
| 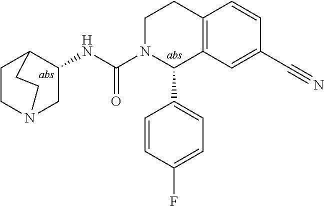 [SMILES: N#Cc1ccc2c(c1)[C@H](c1ccc(F)cc1)N(C(=O)N[C@@H]1CN3CCC1CC3)CC2] | Compound 2038 |
| 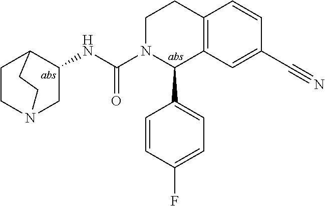 [SMILES: N#Cc1ccc2c(c1)[C@@H](c1ccc(F)cc1)N(C(=O)N[C@@H]1CN3CCC1CC3)CC2] | Compound 2039 |
| 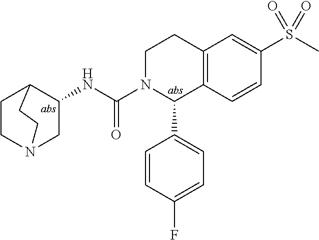 [SMILES: CS(=O)(=O)c1ccc2c(c1)CCN(C(=O)N[C@@H]1CN3CCC1CC3)[C@H]2c1ccc(F)cc1] | Compound 2040 |
| 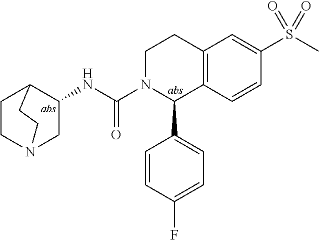 [SMILES: CS(=O)(=O)c1ccc2c(c1)CCN(C(=O)N[C@@H]1CN3CCC1CC3)[C@@H]2c1ccc(F)cc1] | Compound 2041 |
| 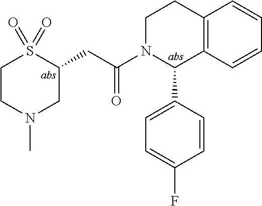 [SMILES: CN1CCS(=O)(=O)[C@H](CC(=O)N2CCc3ccccc3[C@@H]2c2ccc(F)cc2)C1] | Compound 2042 |
| 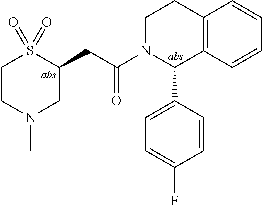 [SMILES: CN1CCS(=O)(=O)[C@@H](CC(=O)N2CCc3ccccc3[C@@H]2c2ccc(F)cc2)C1] | Compound 2043 |
| 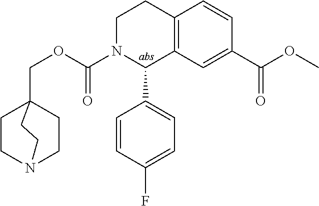 [SMILES: COC(=O)c1ccc2c(c1)[C@H](c1ccc(F)cc1)N(C(=O)OCC13CCN(CC1)CC3)CC2] | Compound 2044 |
| 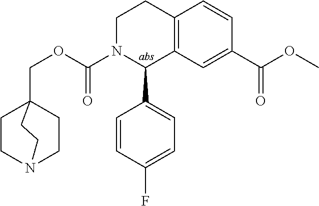 [SMILES: COC(=O)c1ccc2c(c1)[C@@H](c1ccc(F)cc1)N(C(=O)OCC13CCN(CC1)CC3)CC2] | Compound 2045 |
| 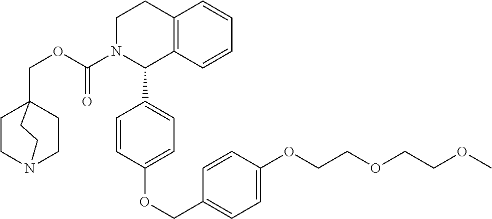 [SMILES: COCCOCCOc1ccc(COc2ccc([C@H]3c4ccccc4CCN3C(=O)OCC34CCN(CC3)CC4)cc2)cc1] | Compound 2046 |
| 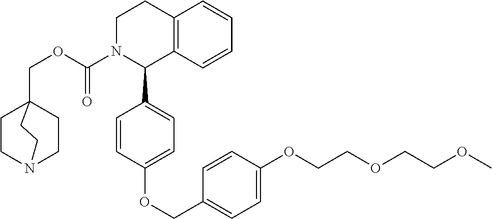 [SMILES: COCCOCCOc1ccc(COc2ccc([C@@H]3c4ccccc4CCN3C(=O)OCC34CCN(CC3)CC4)cc2)cc1] | Compound 2047 |
| 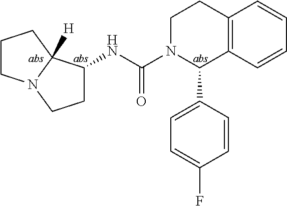 [SMILES: O=C(N[C@@H]1CCN2CCC[C@H]12)N1CCc2ccccc2[C@@H]1c1ccc(F)cc1] | Compound 2048 |
| 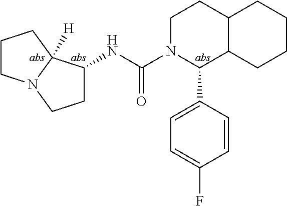 [SMILES: O=C(N[C@@H]1CCN2CCC[C@@H]12)N1CCC2CCCCC2[C@@H]1c1ccc(F)cc1] | Compound 2049 |
| 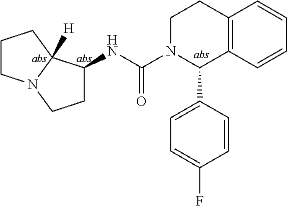 [SMILES: O=C(N[C@H]1CCN2CCC[C@H]12)N1CCc2ccccc2[C@@H]1c1ccc(F)cc1] | Compound 2050 |
| 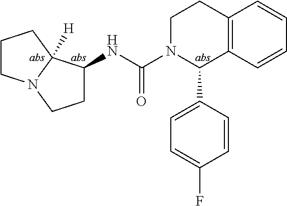 [SMILES: O=C(N[C@H]1CCN2CCC[C@@H]12)N1CCc2ccccc2[C@@H]1c1ccc(F)cc1] | Compound 2051 |
| 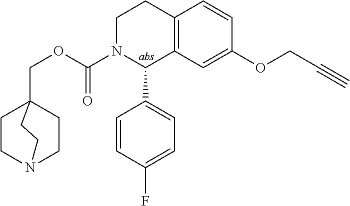 [SMILES: C#CCOc1ccc2c(c1)[C@H](c1ccc(F)cc1)N(C(=O)OCC13CCN(CC1)CC3)CC2] | Compound 2052 |
| 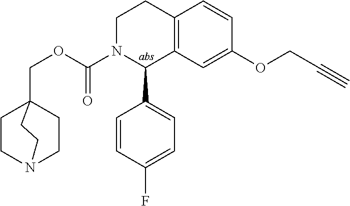 [SMILES: C#CCOc1ccc2c(c1)[C@@H](c1ccc(F)cc1)N(C(=O)OCC13CCN(CC1)CC3)CC2] | Compound 2053 |
| 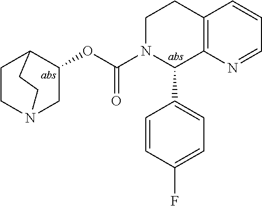 [SMILES: O=C(O[C@@H]1CN2CCC1CC2)N1CCc2cccnc2[C@@H]1c1ccc(F)cc1] | Compound 2054 |
| 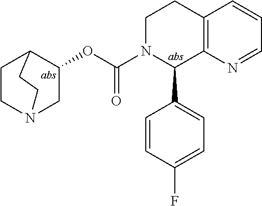 [SMILES: O=C(O[C@@H]1CN2CCC1CC2)N1CCc2cccnc2[C@H]1c1ccc(F)cc1] | Compound 2055 |
| 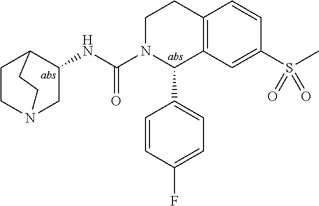 [SMILES: CS(=O)(=O)c1ccc2c(c1)[C@H](c1ccc(F)cc1)N(C(=O)N[C@@H]1CN3CCC1CC3)CC2] | Compound 2056 |
| 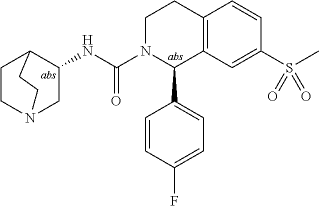 [SMILES: CS(=O)(=O)c1ccc2c(c1)[C@@H](c1ccc(F)cc1)N(C(=O)N[C@@H]1CN3CCC1CC3)CC2] | Compound 2057 |
| 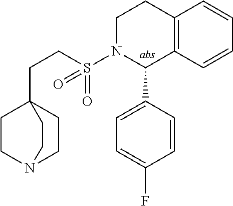 [SMILES: O=S(=O)(CCC12CCN(CC1)CC2)N1CCc2ccccc2[C@@H]1c1ccc(F)cc1] | Compound 2058 |
| 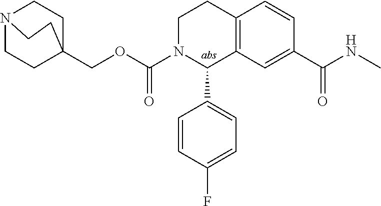 [SMILES: CNC(=O)c1ccc2c(c1)[C@H](c1ccc(F)cc1)N(C(=O)OCC13CCN(CC1)CC3)CC2] | Compound 2059 |
| 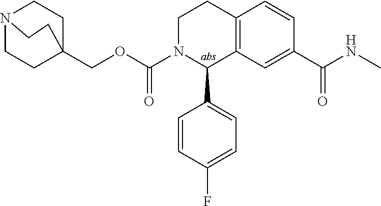 [SMILES: CNC(=O)c1ccc2c(c1)[C@@H](c1ccc(F)cc1)N(C(=O)OCC13CCN(CC1)CC3)CC2] | Compound 2060 |
| 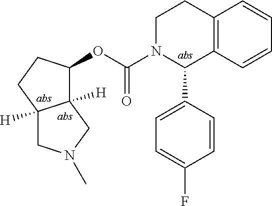 [SMILES: CN1C[C@H]2CC[C@@H](OC(=O)N3CCc4ccccc4[C@@H]3c3ccc(F)cc3)[C@H]2C1] | Compound 2061 |
| 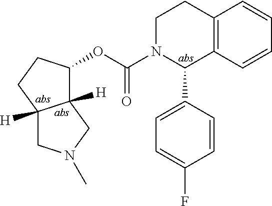 [SMILES: CN1C[C@@H]2CC[C@H](OC(=O)N3CCc4ccccc4[C@@H]3c3ccc(F)cc3)[C@@H]2C1] | Compound 2062 |
| 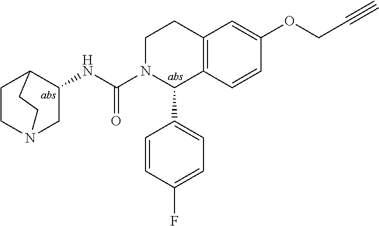 [SMILES: C#CCOc1ccc2c(c1)CCN(C(=O)N[C@@H]1CN3CCC1CC3)[C@H]2c1ccc(F)cc1] | Compound 2063 |
| 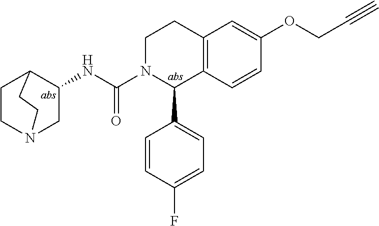 [SMILES: C#CCOc1ccc2c(c1)CCN(C(=O)N[C@@H]1CN3CCC1CC3)[C@@H]2c1ccc(F)cc1] | Compound 2064 |
| 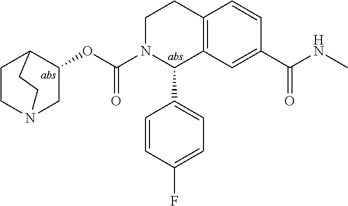 [SMILES: CNC(=O)c1ccc2c(c1)[C@H](c1ccc(F)cc1)N(C(=O)O[C@@H]1CN3CCC1CC3)CC2] | Compound 2065 |
| 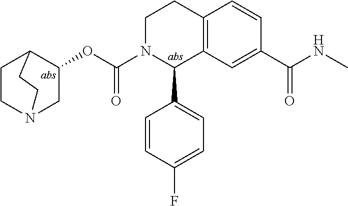 [SMILES: CNC(=O)c1ccc2c(c1)[C@@H](c1ccc(F)cc1)N(C(=O)O[C@@H]1CN3CCC1CC3)CC2] | Compound 2066 |
| 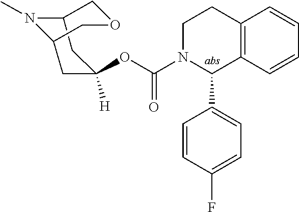 | Compound 2067 |
| 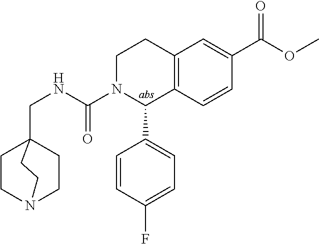 [SMILES: COC(=O)c1ccc2c(c1)CCN(C(=O)NCC13CCN(CC1)CC3)[C@H]2c1ccc(F)cc1] | Compound 2068 |
| 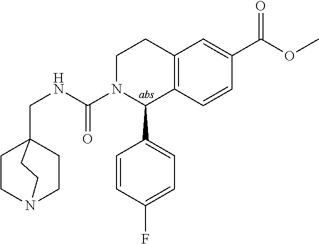 [SMILES: COC(=O)c1ccc2c(c1)CCN(C(=O)NCC13CCN(CC1)CC3)[C@@H]2c1ccc(F)cc1] | Compound 2069 |
| 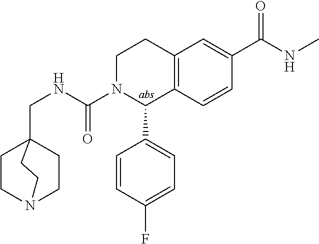 [SMILES: CNC(=O)c1ccc2c(c1)CCN(C(=O)NCC13CCN(CC1)CC3)[C@H]2c1ccc(F)cc1] | Compound 2070 |
| 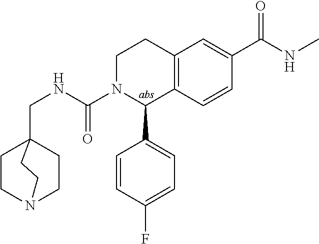 [SMILES: CNC(=O)c1ccc2c(c1)CCN(C(=O)NCC13CCN(CC1)CC3)[C@@H]2c1ccc(F)cc1] | Compound 2071 |
| 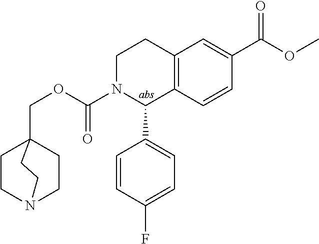 [SMILES: COC(=O)c1ccc2c(c1)CCN(C(=O)OCC13CCN(CC1)CC3)[C@H]2c1ccc(F)cc1] | Compound 2072 |
| 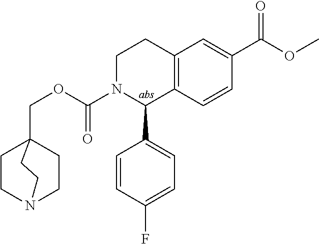 [SMILES: COC(=O)c1ccc2c(c1)CCN(C(=O)OCC13CCN(CC1)CC3)[C@@H]2c1ccc(F)cc1] | Compound 2073 |
| 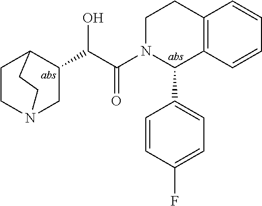 [SMILES: O=C(C(O)[C@@H]1CN2CCC1CC2)N1CCc2ccccc2[C@@H]1c1ccc(F)cc1] | Compound 2074 |
| 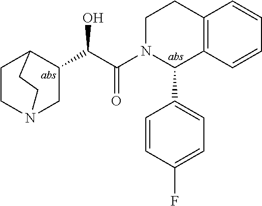 [SMILES: O=C([C@H](O)[C@@H]1CN2CCC1CC2)N1CCc2ccccc2[C@@H]1c1ccc(F)cc1] | Compound 2075 |
| 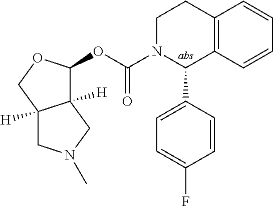 [SMILES: CN1C[C@H]2CO[C@@H](OC(=O)N3CCc4ccccc4[C@@H]3c3ccc(F)cc3)[C@H]2C1] | Compound 2076 |
| 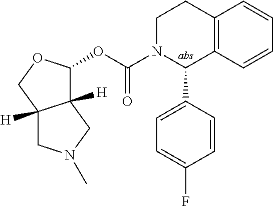 [SMILES: CN1C[C@@H]2CO[C@H](OC(=O)N3CCc4ccccc4[C@@H]3c3ccc(F)cc3)[C@@H]2C1] | Compound 2077 |
| 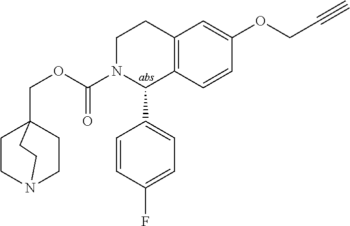 [SMILES: C#CCOc1ccc2c(c1)CCN(C(=O)OCC13CCN(CC1)CC3)[C@H]2c1ccc(F)cc1] | Compound 2078 |
| 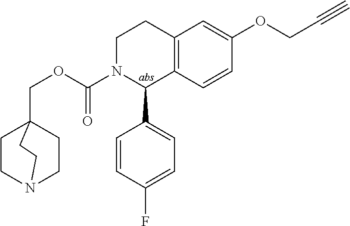 [SMILES: C#CCOc1ccc2c(c1)CCN(C(=O)OCC13CCN(CC1)CC3)[C@@H]2c1ccc(F)cc1] | Compound 2079 |
| 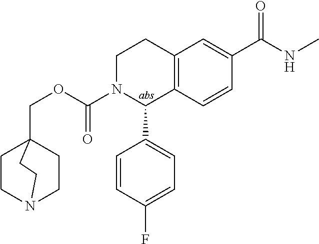 [SMILES: CNC(=O)c1ccc2c(c1)CCN(C(=O)OCC13CCN(CC1)CC3)[C@H]2c1ccc(F)cc1] | Compound 2080 |
| 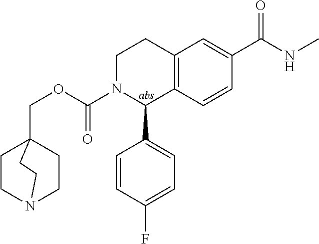 [SMILES: CNC(=O)c1ccc2c(c1)CCN(C(=O)OCC13CCN(CC1)CC3)[C@@H]2c1ccc(F)cc1] | Compound 2081 |
| 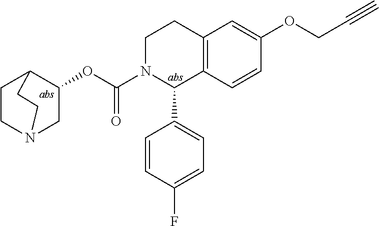 [SMILES: C#CCOc1ccc2c(c1)CCN(C(=O)O[C@@H]1CN3CCC1CC3)[C@H]2c1ccc(F)cc1] | Compound 2082 |
| 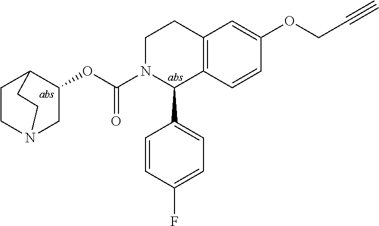 [SMILES: C#CCOc1ccc2c(c1)CCN(C(=O)O[C@@H]1CN3CCC1CC3)[C@@H]2c1ccc(F)cc1] | Compound 2083 |
| 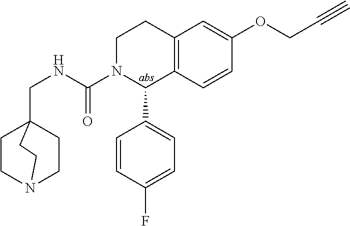 [SMILES: C#CCOc1ccc2c(c1)CCN(C(=O)NCC13CCN(CC1)CC3)[C@H]2c1ccc(F)cc1] | Compound 2084 |
| 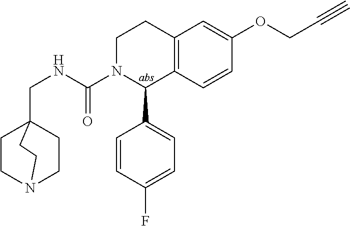 [SMILES: C#CCOc1ccc2c(c1)CCN(C(=O)NCC13CCN(CC1)CC3)[C@@H]2c1ccc(F)cc1] | Compound 2085 |
| 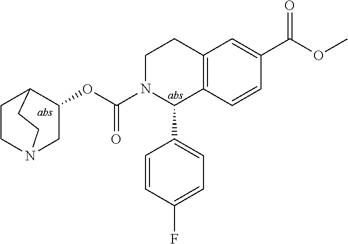 [SMILES: COC(=O)c1ccc2c(c1)CCN(C(=O)O[C@@H]1CN3CCC1CC3)[C@H]2c1ccc(F)cc1] | Compound 2086 |
| 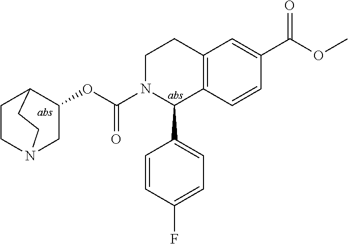 [SMILES: COC(=O)c1ccc2c(c1)CCN(C(=O)O[C@@H]1CN3CCC1CC3)[C@@H]2c1ccc(F)cc1] | Compound 2087 |
| 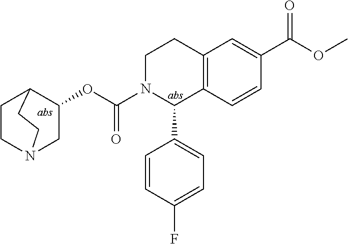 [SMILES: COC(=O)c1ccc2c(c1)CCN(C(=O)O[C@@H]1CN3CCC1CC3)[C@H]2c1ccc(F)cc1] | Compound 2088 |
| 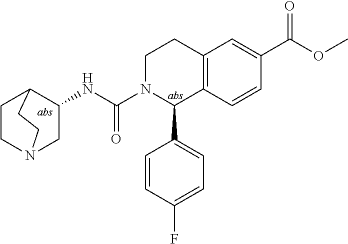 [SMILES: COC(=O)c1ccc2c(c1)CCN(C(=O)N[C@@H]1CN3CCC1CC3)[C@@H]2c1ccc(F)cc1] | Compound 2089 |
| 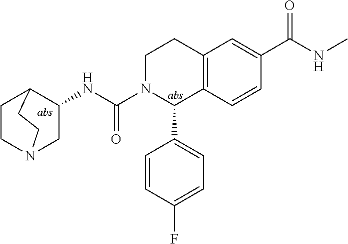 [SMILES: CNC(=O)c1ccc2c(c1)CCN(C(=O)N[C@@H]1CN3CCC1CC3)[C@H]2c1ccc(F)cc1] | Compound 2090 |
| 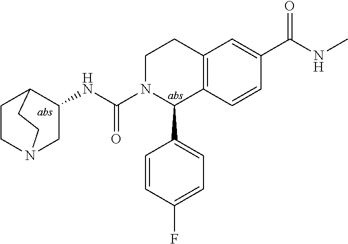 [SMILES: CNC(=O)c1ccc2c(c1)CCN(C(=O)N[C@@H]1CN3CCC1CC3)[C@@H]2c1ccc(F)cc1] | Compound 2091 |
| 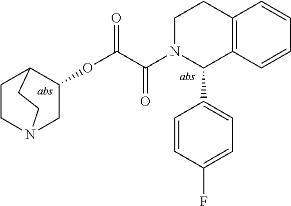 [SMILES: O=C(O[C@@H]1CN2CCC1CC2)C(=O)N1CCc2ccccc2[C@@H]1c1ccc(F)cc1] | Compound 2092 |
| 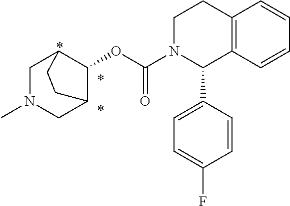 | Compound 2093 |
| 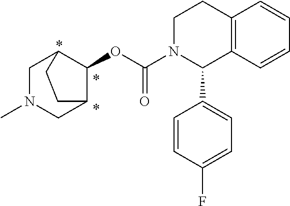 | Compound 2094 |
| 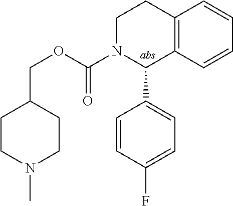 [SMILES: CN1CCC(COC(=O)N2CCc3ccccc3[C@@H]2c2ccc(F)cc2)CC1] | Compound 2095 |
| 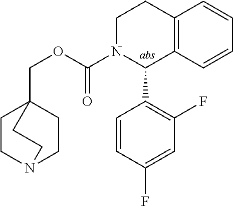 [SMILES: O=C(OCC12CCN(CC1)CC2)N1CCc2ccccc2[C@@H]1c1ccc(F)cc1F] | Compound 2096 |
| 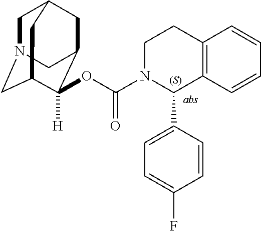 | Compound 2097 |
| 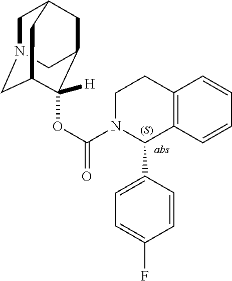 | Compound 2098 |
| 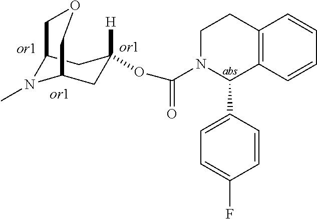 | Compound 2099 |
| 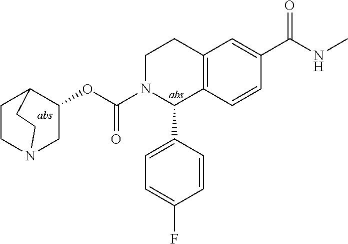 [SMILES: CNC(=O)c1ccc2c(c1)CCN(C(=O)O[C@@H]1CN3CCC1CC3)[C@H]2c1ccc(F)cc1] | Compound 2100 |
| 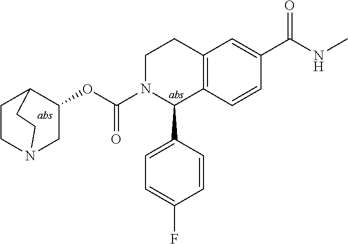 [SMILES: CNC(=O)c1ccc2c(c1)CCN(C(=O)O[C@@H]1CN3CCC1CC3)[C@@H]2c1ccc(F)cc1] | Compound 2101 |
| 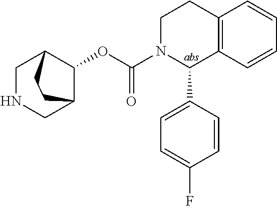 [SMILES: O=C(O[C@H]1[C@@H]2CC[C@H]1CNC2)N1CCc2ccccc2[C@@H]1c1ccc(F)cc1] | Compound 2102 |
| 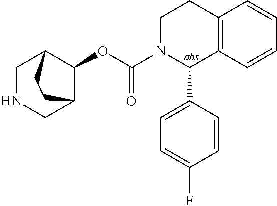 [SMILES: O=C(O[C@@H]1[C@@H]2CC[C@H]1CNC2)N1CCc2ccccc2[C@@H]1c1ccc(F)cc1] | Compound 2103 |
| 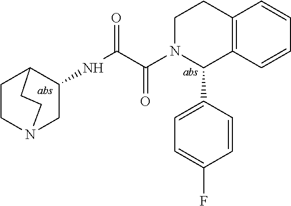 [SMILES: O=C(N[C@@H]1CN2CCC1CC2)C(=O)N1CCc2ccccc2[C@@H]1c1ccc(F)cc1] | Compound 2104 |
| 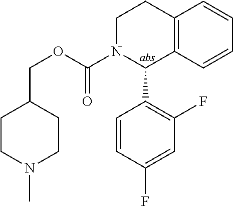 [SMILES: CN1CCC(COC(=O)N2CCc3ccccc3[C@@H]2c2ccc(F)cc2F)CC1] | Compound 2105 |
| 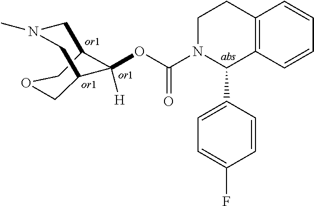 | Compound 2106 |
| 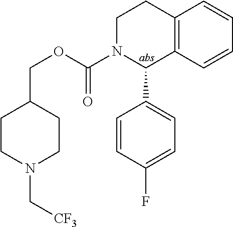 [SMILES: O=C(OCC1CCN(CC(F)(F)F)CC1)N1CCc2ccccc2[C@@H]1c1ccc(F)cc1] | Compound 2107 |
| 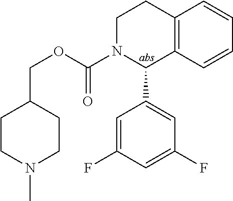 [SMILES: CN1CCC(COC(=O)N2CCc3ccccc3[C@@H]2c2cc(F)cc(F)c2)CC1] | Compound 2108 |
| 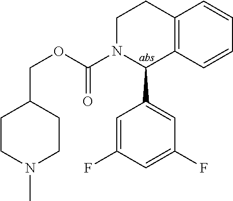 [SMILES: CN1CCC(COC(=O)N2CCc3ccccc3[C@H]2c2cc(F)cc(F)c2)CC1] | Compound 2109 |
| 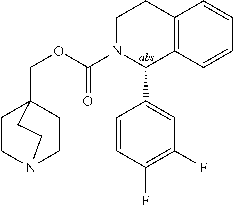 [SMILES: O=C(OCC12CCN(CC1)CC2)N1CCc2ccccc2[C@@H]1c1ccc(F)c(F)c1] | Compound 2110 |
| 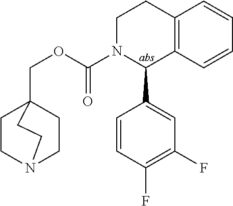 [SMILES: O=C(OCC12CCN(CC1)CC2)N1CCc2ccccc2[C@H]1c1ccc(F)c(F)c1] | Compound 2111 |
| 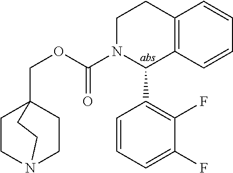 [SMILES: O=C(OCC12CCN(CC1)CC2)N1CCc2ccccc2[C@@H]1c1cccc(F)c1F] | Compound 2112 |
| 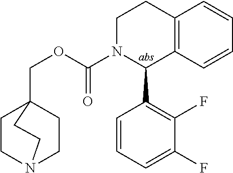 [SMILES: O=C(OCC12CCN(CC1)CC2)N1CCc2ccccc2[C@H]1c1cccc(F)c1F] | Compound 2113 |
| 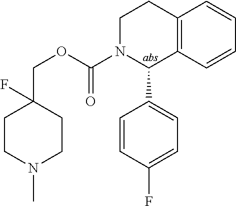 [SMILES: CN1CCC(F)(COC(=O)N2CCc3ccccc3[C@@H]2c2ccc(F)cc2)CC1] | Compound 2114 |
| 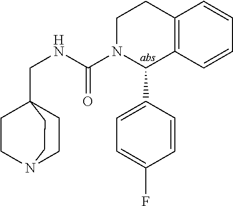 [SMILES: O=C(NCC12CCN(CC1)CC2)N1CCc2ccccc2[C@@H]1c1ccc(F)cc1] | Compound 2178 |
| 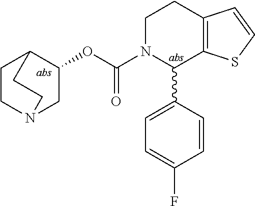 [SMILES: O=C(O[C@@H]1CN2CCC1CC2)N1CCc2ccsc2C1c1ccc(F)cc1] | Compound 2122 |
| 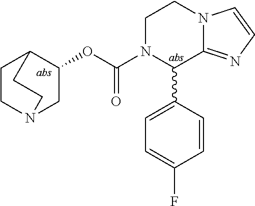 [SMILES: O=C(O[C@@H]1CN2CCC1CC2)N1CCn2ccnc2C1c1ccc(F)cc1] | Compound 2123 |
| 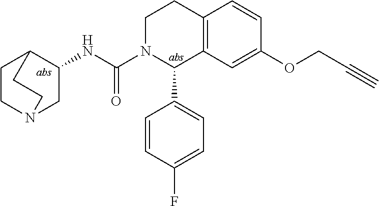 [SMILES: C#CCOc1ccc2c(c1)[C@H](c1ccc(F)cc1)N(C(=O)N[C@@H]1CN3CCC1CC3)CC2] | Compound 2115 |
| 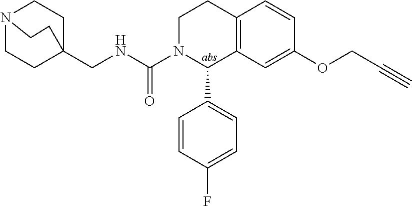 [SMILES: C#CCOc1ccc2c(c1)[C@H](c1ccc(F)cc1)N(C(=O)NCC13CCN(CC1)CC3)CC2] | Compound 2192 |
| 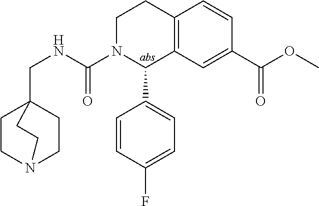 [SMILES: COC(=O)c1ccc2c(c1)[C@H](c1ccc(F)cc1)N(C(=O)NCC13CCN(CC1)CC3)CC2] | Compound 2117 |
| 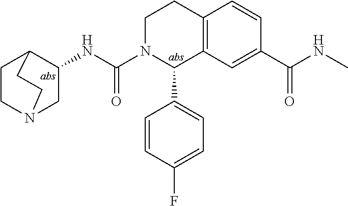 [SMILES: CNC(=O)c1ccc2c(c1)[C@H](c1ccc(F)cc1)N(C(=O)N[C@@H]1CN3CCC1CC3)CC2] | Compound 2118 |
| 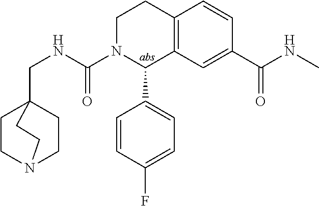 [SMILES: CNC(=O)c1ccc2c(c1)[C@H](c1ccc(F)cc1)N(C(=O)NCC13CCN(CC1)CC3)CC2] | Compound 2119 |
| 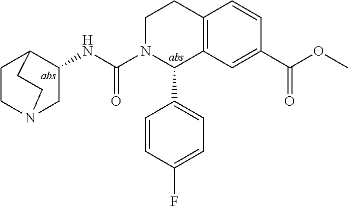 [SMILES: COC(=O)c1ccc2c(c1)[C@H](c1ccc(F)cc1)N(C(=O)N[C@@H]1CN3CCC1CC3)CC2] | Compound 2116 |
| 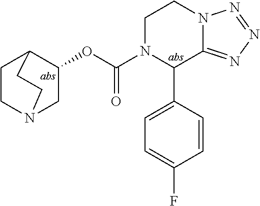 [SMILES: O=C(O[C@@H]1CN2CCC1CC2)N1CCn2nnnc2C1c1ccc(F)cc1] | Compound 2124 |
| 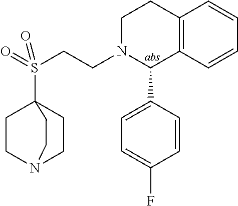 [SMILES: O=S(=O)(CCN1CCc2ccccc2[C@@H]1c1ccc(F)cc1)C12CCN(CC1)CC2] | Compound 2131 |
| 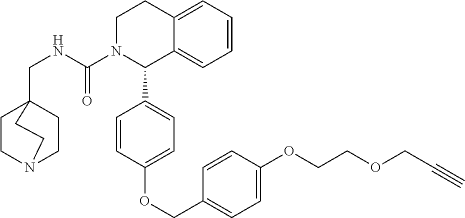 [SMILES: C#CCOCCOc1ccc(COc2ccc([C@H]3c4ccccc4CCN3C(=O)NCC34CCN(CC3)CC4)cc2)cc1] | Compound 2125 |
| 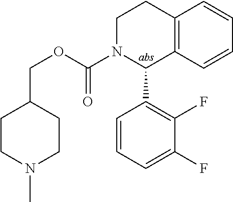 [SMILES: CN1CCC(COC(=O)N2CCc3ccccc3[C@@H]2c2cccc(F)c2F)CC1] | Compound 2134 |
| 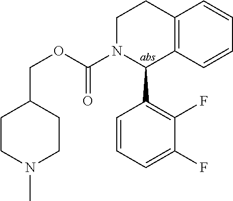 [SMILES: CN1CCC(COC(=O)N2CCc3ccccc3[C@H]2c2cccc(F)c2F)CC1] | Compound 2135 |
| 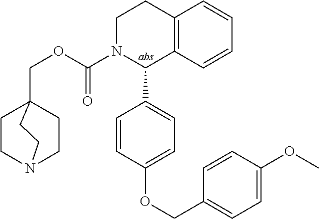 [SMILES: COc1ccc(COc2ccc([C@H]3c4ccccc4CCN3C(=O)OCC34CCN(CC3)CC4)cc2)cc1] | Compound 2136 |
| 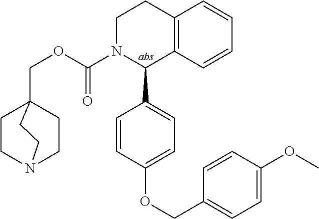 [SMILES: COc1ccc(COc2ccc([C@@H]3c4ccccc4CCN3C(=O)OCC34CCN(CC3)CC4)cc2)cc1] | Compound 2137 |
| 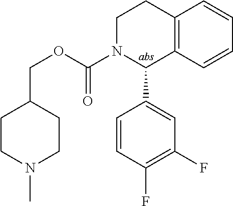 [SMILES: CN1CCC(COC(=O)N2CCc3ccccc3[C@@H]2c2ccc(F)c(F)c2)CC1] | Compound 2138 |
| 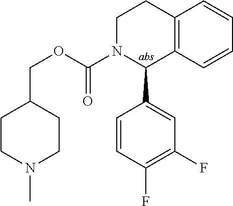 [SMILES: CN1CCC(COC(=O)N2CCc3ccccc3[C@H]2c2ccc(F)c(F)c2)CC1] | Compound 2139 |
| 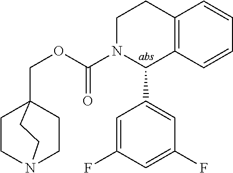 [SMILES: O=C(OCC12CCN(CC1)CC2)N1CCc2ccccc2[C@@H]1c1cc(F)cc(F)c1] | Compound 2140 |
| 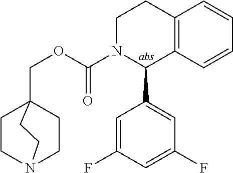 [SMILES: O=C(OCC12CCN(CC1)CC2)N1CCc2ccccc2[C@H]1c1cc(F)cc(F)c1] | Compound 2141 |
| 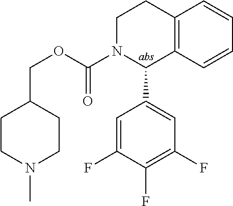 [SMILES: CN1CCC(COC(=O)N2CCc3ccccc3[C@@H]2c2cc(F)c(F)c(F)c2)CC1] | Compound 2142 |
| 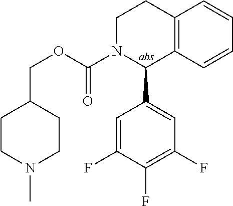 [SMILES: CN1CCC(COC(=O)N2CCc3ccccc3[C@H]2c2cc(F)c(F)c(F)c2)CC1] | Compound 2143 |
| 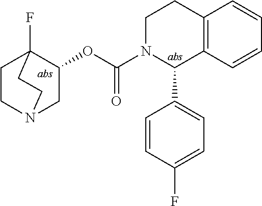 [SMILES: O=C(O[C@@H]1CN2CCC1(F)CC2)N1CCc2ccccc2[C@@H]1c1ccc(F)cc1] | Compound 2126 |
| 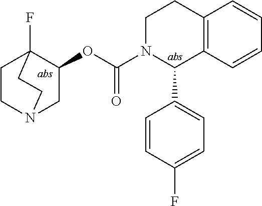 [SMILES: O=C(O[C@H]1CN2CCC1(F)CC2)N1CCc2ccccc2[C@@H]1c1ccc(F)cc1] | Compound 2127 |
| 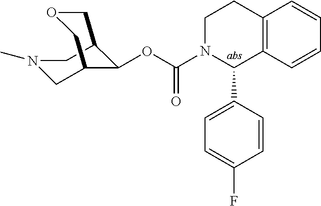 | Compound 2144 |
| 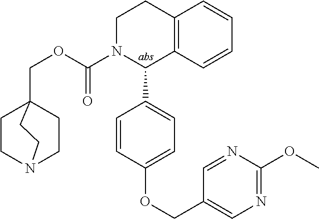 [SMILES: COc1ncc(COc2ccc([C@H]3c4ccccc4CCN3C(=O)OCC34CCN(CC3)CC4)cc2)cn1] | Compound 2145 |
| 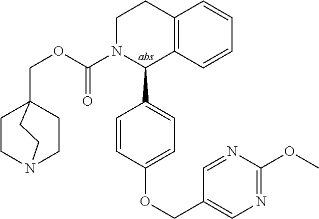 [SMILES: COc1ncc(COc2ccc([C@@H]3c4ccccc4CCN3C(=O)OCC34CCN(CC3)CC4)cc2)cn1] | Compound 2146 |
| 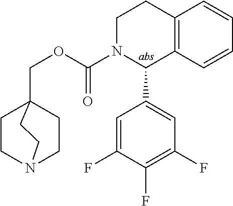 [SMILES: O=C(OCC12CCN(CC1)CC2)N1CCc2ccccc2[C@@H]1c1cc(F)c(F)c(F)c1] | Compound 2147 |
| 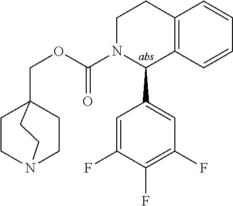 [SMILES: O=C(OCC12CCN(CC1)CC2)N1CCc2ccccc2[C@H]1c1cc(F)c(F)c(F)c1] | Compound 2148 |
| 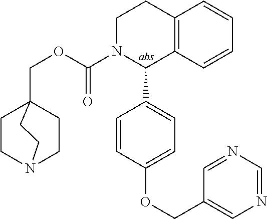 [SMILES: O=C(OCC12CCN(CC1)CC2)N1CCc2ccccc2[C@@H]1c1ccc(OCc2cncnc2)cc1] | Compound 2149 |
| 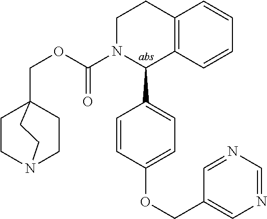 [SMILES: O=C(OCC12CCN(CC1)CC2)N1CCc2ccccc2[C@H]1c1ccc(OCc2cncnc2)cc1] | Compound 2150 |
| 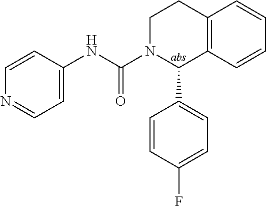 [SMILES: O=C(Nc1ccncc1)N1CCc2ccccc2[C@@H]1c1ccc(F)cc1] | Compound 2151 |
| 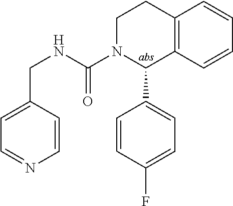 [SMILES: O=C(NCc1ccncc1)N1CCc2ccccc2[C@@H]1c1ccc(F)cc1] | Compound 2152 |
| 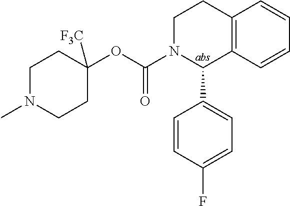 [SMILES: CN1CCC(OC(=O)N2CCc3ccccc3[C@@H]2c2ccc(F)cc2)(C(F)(F)F)CC1] | Compound 2128 |
| 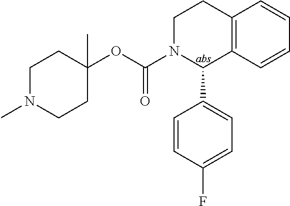 [SMILES: CN1CCC(C)(OC(=O)N2CCc3ccccc3[C@@H]2c2ccc(F)cc2)CC1] | Compound 2129 |
| 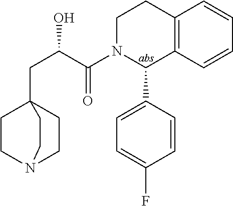 [SMILES: O=C([C@@H](O)CC12CCN(CC1)CC2)N1CCc2ccccc2[C@@H]1c1ccc(F)cc1] | Compound 2153 |
| 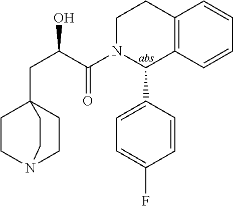 [SMILES: O=C([C@H](O)CC12CCN(CC1)CC2)N1CCc2ccccc2[C@@H]1c1ccc(F)cc1] | Compound 2154 |
| 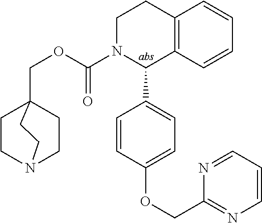 [SMILES: O=C(OCC12CCN(CC1)CC2)N1CCc2ccccc2[C@@H]1c1ccc(OCc2ncccn2)cc1] | Compound 2155 |
| 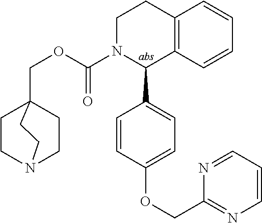 [SMILES: O=C(OCC12CCN(CC1)CC2)N1CCc2ccccc2[C@H]1c1ccc(OCc2ncccn2)cc1] | Compound 2156 |
| 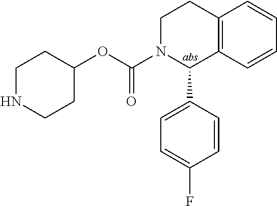 [SMILES: O=C(OC1CCNCC1)N1CCc2ccccc2[C@@H]1c1ccc(F)cc1] | Compound 2161 |
| 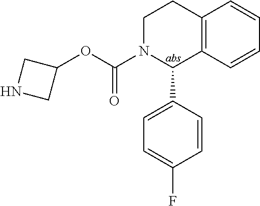 [SMILES: O=C(OC1CNC1)N1CCc2ccccc2[C@@H]1c1ccc(F)cc1] | Compound 2162 |
| 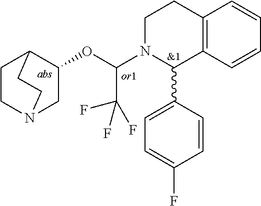 [SMILES: Fc1ccc(C2c3ccccc3CCN2C(O[C@@H]2CN3CCC2CC3)C(F)(F)F)cc1] | Compound 2133 |
| 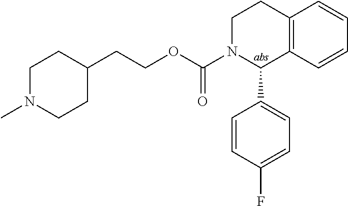 [SMILES: CN1CCC(CCOC(=O)N2CCc3ccccc3[C@@H]2c2ccc(F)cc2)CC1] | Compound 2163 |
| 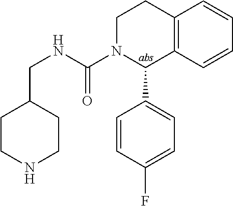 [SMILES: O=C(NCC1CCNCC1)N1CCc2ccccc2[C@@H]1c1ccc(F)cc1] | Compound 2165 |
| 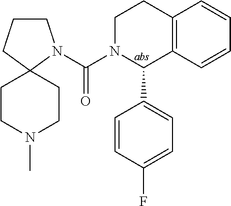 [SMILES: CN1CCC2(CCCN2C(=O)N2CCc3ccccc3[C@@H]2c2ccc(F)cc2)CC1] | Compound 2120 |
| 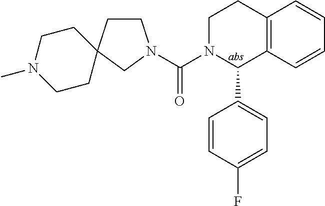 [SMILES: CN1CCC2(CC1)CCN(C(=O)N1CCc3ccccc3[C@@H]1c1ccc(F)cc1)C2] | Compound 2121 |
| 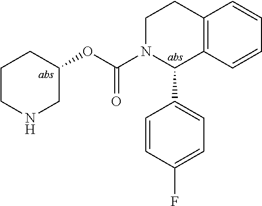 [SMILES: O=C(O[C@H]1CCCNC1)N1CCc2ccccc2[C@@H]1c1ccc(F)cc1] | Compound 2168 |
| 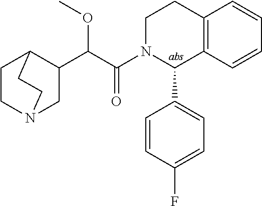 [SMILES: COC(C(=O)N1CCc2ccccc2[C@@H]1c1ccc(F)cc1)C1CN2CCC1CC2] | Compound 2175 |
| 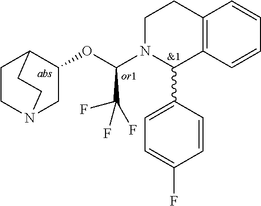 [SMILES: Fc1ccc(C2c3ccccc3CCN2[C@H](O[C@@H]2CN3CCC2CC3)C(F)(F)F)cc1] | Compound 2132 |
| 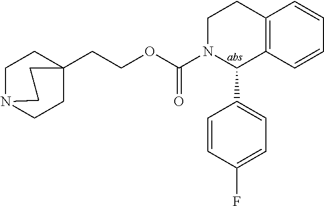 [SMILES: O=C(OCCC12CCN(CC1)CC2)N1CCc2ccccc2[C@@H]1c1ccc(F)cc1] | Compound 2130 |
| 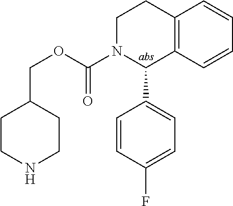 [SMILES: O=C(OCC1CCNCC1)N1CCc2ccccc2[C@@H]1c1ccc(F)cc1] | Compound 2170 |
| 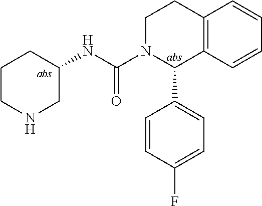 [SMILES: O=C(N[C@H]1CCCNC1)N1CCc2ccccc2[C@@H]1c1ccc(F)cc1] | Compound 2171 |
| 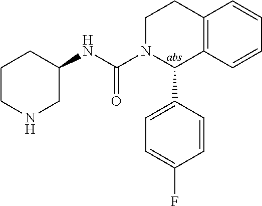 [SMILES: O=C(N[C@@H]1CCCNC1)N1CCc2ccccc2[C@@H]1c1ccc(F)cc1] | Compound 2172 |
| 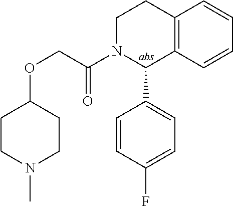 [SMILES: CN1CCC(OCC(=O)N2CCc3ccccc3[C@@H]2c2ccc(F)cc2)CC1] | Compound 2173 |
| 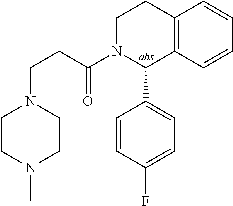 [SMILES: CN1CCN(CCC(=O)N2CCc3ccccc3[C@@H]2c2ccc(F)cc2)CC1] | Compound 2174 |
| 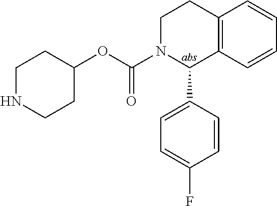 [SMILES: O=C(OC1CCNCC1)N1CCc2ccccc2[C@@H]1c1ccc(F)cc1] | Compound 2176 |
| 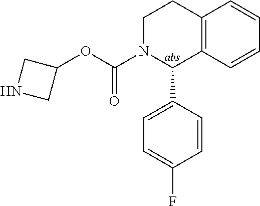 [SMILES: O=C(OC1CNC1)N1CCc2ccccc2[C@@H]1c1ccc(F)cc1] | Compound 2177 |
| 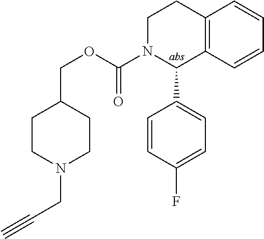 [SMILES: C#CCN1CCC(COC(=O)N2CCc3ccccc3[C@@H]2c2ccc(F)cc2)CC1] | Compound 2194 |
| 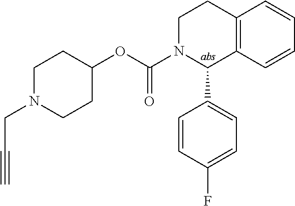 [SMILES: C#CCN1CCC(OC(=O)N2CCc3ccccc3[C@@H]2c2ccc(F)cc2)CC1] | Compound 2195 |
| 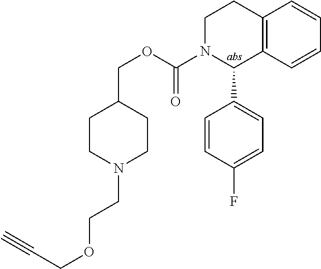 [SMILES: C#CCOCCN1CCC(COC(=O)N2CCc3ccccc3[C@@H]2c2ccc(F)cc2)CC1] | Compound 2196 |
| 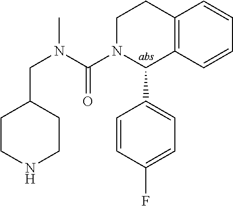 [SMILES: CN(CC1CCNCC1)C(=O)N1CCc2ccccc2[C@@H]1c1ccc(F)cc1] | Compound 2182 |
| 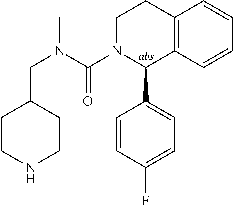 [SMILES: CN(CC1CCNCC1)C(=O)N1CCc2ccccc2[C@H]1c1ccc(F)cc1] | Compound 2183 |
| 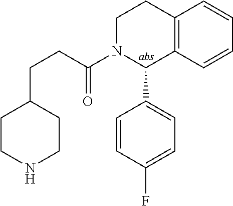 [SMILES: O=C(CCC1CCNCC1)N1CCc2ccccc2[C@@H]1c1ccc(F)cc1] | Compound 2184 |
| 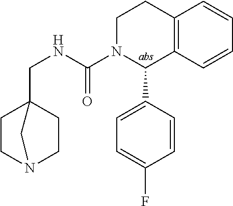 [SMILES: O=C(NCC12CCN(CC1)C2)N1CCc2ccccc2[C@@H]1c1ccc(F)cc1] | Compound 2193 |
| 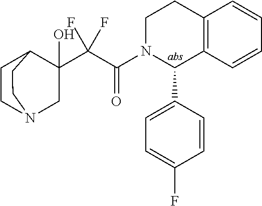 [SMILES: O=C(N1CCc2ccccc2[C@@H]1c1ccc(F)cc1)C(F)(F)C1(O)CN2CCC1CC2] | Compound 2198 |
| 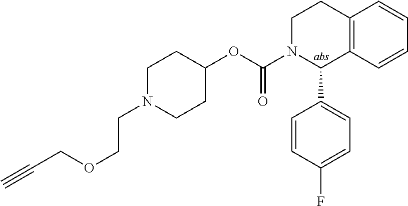 [SMILES: C#CCOCCN1CCC(OC(=O)N2CCc3ccccc3[C@@H]2c2ccc(F)cc2)CC1] | Compound 2197 |
| 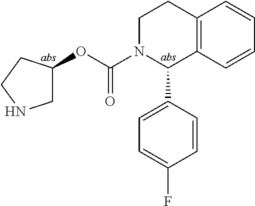 [SMILES: O=C(O[C@@H]1CCNC1)N1CCc2ccccc2[C@@H]1c1ccc(F)cc1] | Compound 2179 |
| 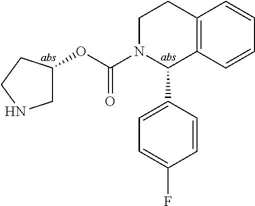 [SMILES: O=C(O[C@H]1CCNC1)N1CCc2ccccc2[C@@H]1c1ccc(F)cc1] | Compound 2180 |
| 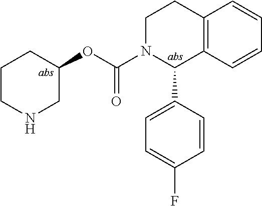 [SMILES: O=C(O[C@@H]1CCCNC1)N1CCc2ccccc2[C@@H]1c1ccc(F)cc1] | Compound 2181 |
| 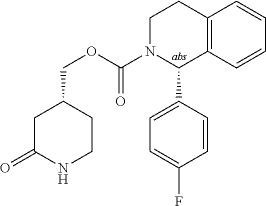 [SMILES: O=C1C[C@H](COC(=O)N2CCc3ccccc3[C@@H]2c2ccc(F)cc2)CCN1] | Compound 2185 |
| 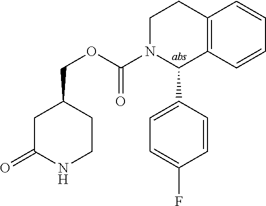 [SMILES: O=C1C[C@@H](COC(=O)N2CCc3ccccc3[C@@H]2c2ccc(F)cc2)CCN1] | Compound 2186 |
| 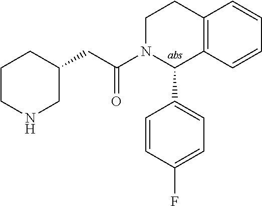 [SMILES: O=C(C[C@H]1CCCNC1)N1CCc2ccccc2[C@@H]1c1ccc(F)cc1] | Compound 2187 |
| 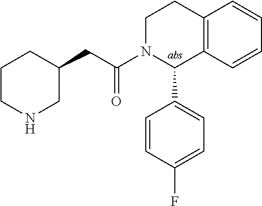 [SMILES: O=C(C[C@@H]1CCCNC1)N1CCc2ccccc2[C@@H]1c1ccc(F)cc1] | Compound 2188 |
| 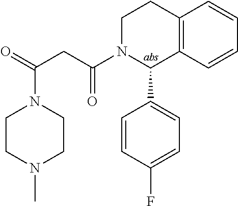 [SMILES: CN1CCN(C(=O)CC(=O)N2CCc3ccccc3[C@@H]2c2ccc(F)cc2)CC1] | Compound 2189 |
| 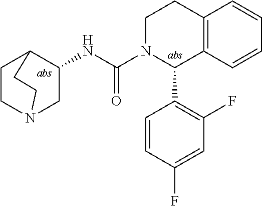 [SMILES: O=C(N[C@@H]1CN2CCC1CC2)N1CCc2ccccc2[C@@H]1c1ccc(F)cc1F] | Compound 2191 |
| 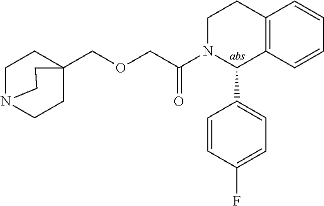 [SMILES: O=C(COCC12CCN(CC1)CC2)N1CCc2ccccc2[C@@H]1c1ccc(F)cc1] | Compound 2190 |

Other compounds contemplated include those listed in Table B, or a pharmaceutically acceptable salt thereof:

TABLE B

| Structure | Compound No. |
|---|---|
| 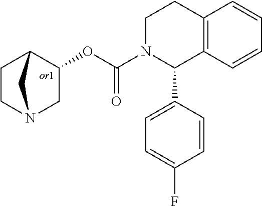 [SMILES: O=C(O[C@@H]1C[N@]2CC[C@H]1C2)N1CCc2ccccc2[C@@H]1c1ccc(F)cc1] | Compound 2202 |
| 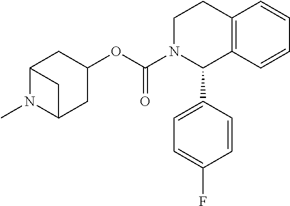 [SMILES: CN1C2CC(OC(=O)N3CCc4ccccc4[C@@H]3c3ccc(F)cc3)CC1C2] | Compound 2560 |
| 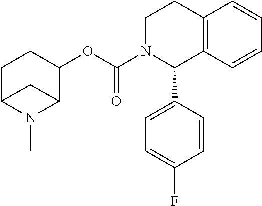 [SMILES: CN1C2CCC(OC(=O)N3CCc4ccccc4[C@@H]3c3ccc(F)cc3)C1C2] | Compound 2561 |
| 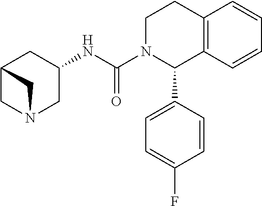 [SMILES: O=C(N[C@H]1C[C@H]2C[N@@](C1)C2)N1CCc2ccccc2[C@@H]1c1ccc(F)cc1] | Compound 2562 |
| 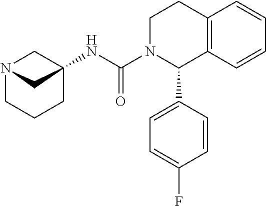 | Compound 2563 |
| 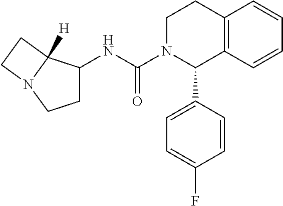 [SMILES: O=C(NC1CCN2CC[C@H]12)N1CCc2ccccc2[C@@H]1c1ccc(F)cc1] | Compound 2564 |
| 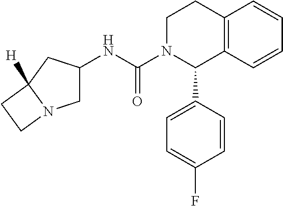 [SMILES: O=C(NC1C[C@H]2CCN2C1)N1CCc2ccccc2[C@@H]1c1ccc(F)cc1] | Compound 2565 |
| 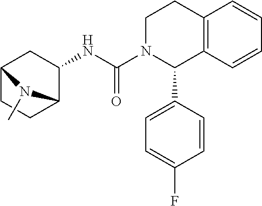 [SMILES: CN1[C@@H]2CC[C@H]1[C@@H](NC(=O)N1CCc3ccccc3[C@@H]1c1ccc(F)cc1)C2] | Compound 2224 |
| 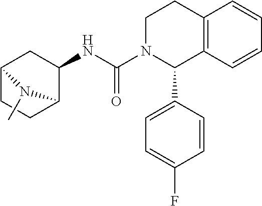 [SMILES: CN1[C@H]2CC[C@@H]1[C@H](NC(=O)N1CCc3ccccc3[C@@H]1c1ccc(F)cc1)C2] | Compound 2225 |
| 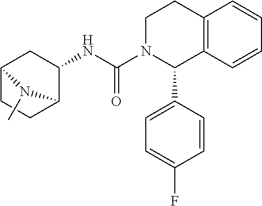 [SMILES: CN1[C@H]2CC[C@@H]1[C@@H](NC(=O)N1CCc3ccccc3[C@@H]1c1ccc(F)cc1)C2] | Compound 2226 |
| 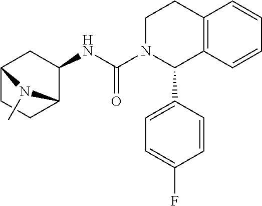 [SMILES: CN1[C@@H]2CC[C@H]1[C@H](NC(=O)N1CCc3ccccc3[C@@H]1c1ccc(F)cc1)C2] | Compound 2227 |
| 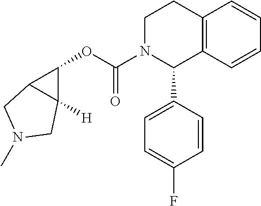 [SMILES: CN1CC2[C@H](C1)[C@H]2OC(=O)N1CCc2ccccc2[C@@H]1c1ccc(F)cc1] | Compound 2566 |
| 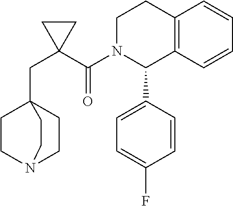 [SMILES: O=C(N1CCc2ccccc2[C@@H]1c1ccc(F)cc1)C1(CC23CCN(CC2)CC3)CC1] | Compound 2567 |
| 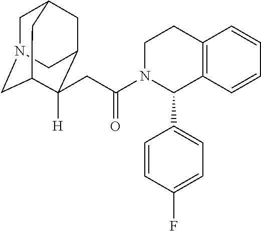 [SMILES: O=C(CC1C2CC3CC1CN(C3)C2)N1CCc2ccccc2[C@@H]1c1ccc(F)cc1] | Compound 2568 |
| 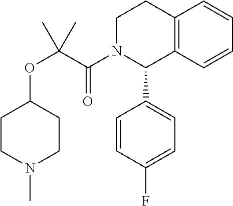 [SMILES: CN1CCC(OC(C)(C)C(=O)N2CCc3ccccc3[C@@H]2c2ccc(F)cc2)CC1] | Compound 2228 |
| 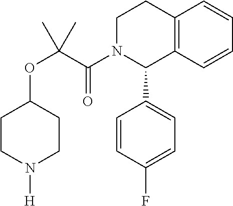 [SMILES: CC(C)(OC1CCNCC1)C(=O)N1CCc2ccccc2[C@@H]1c1ccc(F)cc1] | Compound 2229 |
| 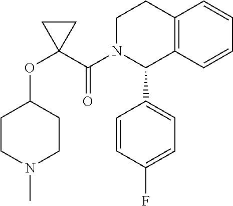 [SMILES: CN1CCC(OC2(C(=O)N3CCc4ccccc4[C@@H]3c3ccc(F)cc3)CC2)CC1] | Compound 2247 |
| 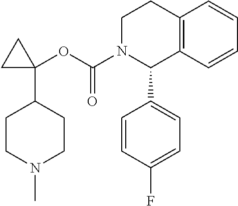 [SMILES: CN1CCC(C2(OC(=O)N3CCc4ccccc4[C@@H]3c3ccc(F)cc3)CC2)CC1] | Compound 2215 |
| 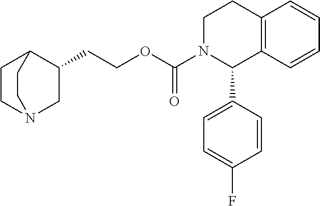 [SMILES: O=C(OCC[C@@H]1CN2CCC1CC2)N1CCc2ccccc2[C@@H]1c1ccc(F)cc1] | Compound 2206 |
| 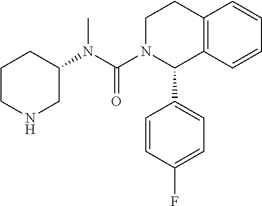 [SMILES: CN(C(=O)N1CCc2ccccc2[C@@H]1c1ccc(F)cc1)[C@H]1CCCNC1] | Compound 2211 |
| 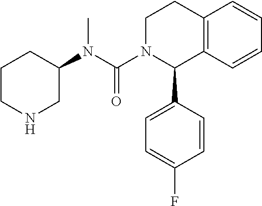 [SMILES: CN(C(=O)N1CCc2ccccc2[C@H]1c1ccc(F)cc1)[C@@H]1CCCNC1] | Compound 2212 |
| 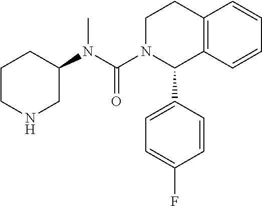 [SMILES: CN(C(=O)N1CCc2ccccc2[C@@H]1c1ccc(F)cc1)[C@@H]1CCCNC1] | Compound 2213 |
| 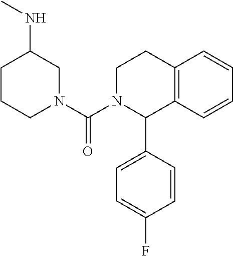 [SMILES: CNC1CCCN(C(=O)N2CCc3ccccc3C2c2ccc(F)cc2)C1] | Compound 2214 |
| 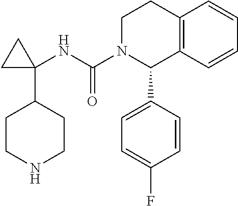 [SMILES: O=C(NC1(C2CCNCC2)CC1)N1CCc2ccccc2[C@@H]1c1ccc(F)cc1] | Compound 2208 |
| 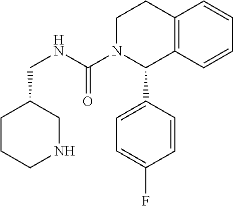 [SMILES: O=C(NC[C@H]1CCCNC1)N1CCc2ccccc2[C@@H]1c1ccc(F)cc1] | Compound 2220 |
| 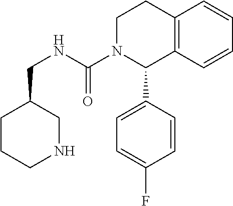 [SMILES: O=C(NC[C@@H]1CCCNC1)N1CCc2ccccc2[C@@H]1c1ccc(F)cc1] | Compound 2221 |
| 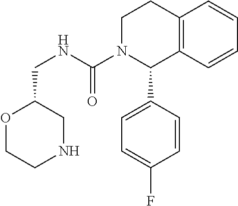 [SMILES: O=C(NC[C@@H]1CNCCO1)N1CCc2ccccc2[C@@H]1c1ccc(F)cc1] | Compound 2209 |
| 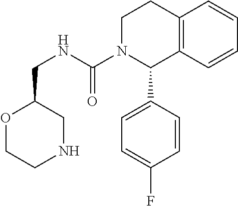 [SMILES: O=C(NC[C@H]1CNCCO1)N1CCc2ccccc2[C@@H]1c1ccc(F)cc1] | Compound 2210 |
| 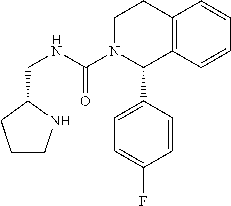 [SMILES: O=C(NC[C@H]1CCCN1)N1CCc2ccccc2[C@@H]1c1ccc(F)cc1] | Compound 2216 |
| 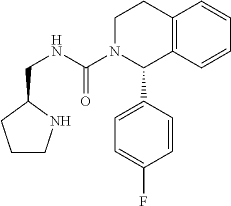 [SMILES: O=C(NC[C@@H]1CCCN1)N1CCc2ccccc2[C@@H]1c1ccc(F)cc1] | Compound 2217 |
| 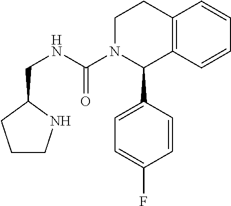 [SMILES: O=C(NC[C@@H]1CCCN1)N1CCc2ccccc2[C@H]1c1ccc(F)cc1] | Compound 2218 |
| 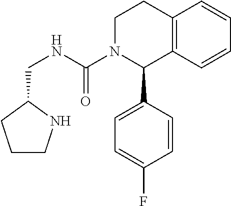 [SMILES: O=C(NC[C@H]1CCCN1)N1CCc2ccccc2[C@H]1c1ccc(F)cc1] | Compound 2219 |
| 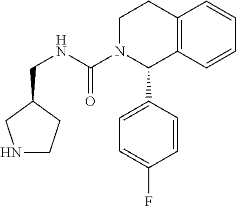 [SMILES: O=C(NC[C@H]1CCNC1)N1CCc2ccccc2[C@@H]1c1ccc(F)cc1] | Compound 2232 |
| 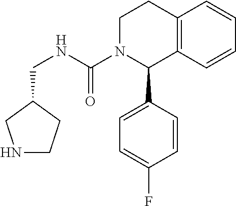 [SMILES: O=C(NC[C@@H]1CCNC1)N1CCc2ccccc2[C@H]1c1ccc(F)cc1] | Compound 2233 |
| 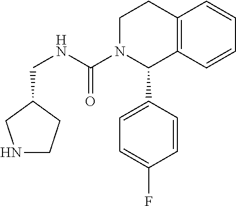 [SMILES: O=C(NC[C@@H]1CCNC1)N1CCc2ccccc2[C@@H]1c1ccc(F)cc1] | Compound 2234 |
| 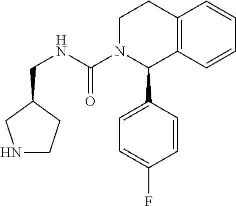 [SMILES: O=C(NC[C@H]1CCNC1)N1CCc2ccccc2[C@H]1c1ccc(F)cc1] | Compound 2235 |
| 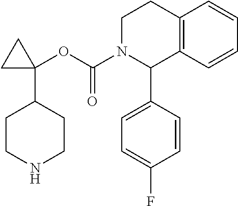 [SMILES: O=C(OC1(C2CCNCC2)CC1)N1CCc2ccccc2C1c1ccc(F)cc1] | Compound 2500 |
| 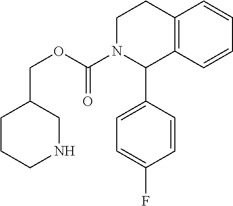 [SMILES: O=C(OCC1CCCNC1)N1CCc2ccccc2C1c1ccc(F)cc1] | Compound 2501 |
| 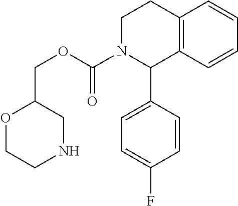 [SMILES: O=C(OCC1CNCCO1)N1CCc2ccccc2C1c1ccc(F)cc1] | Compound 2502 |
| 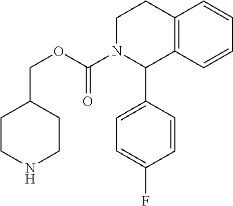 [SMILES: O=C(OCC1CCNCC1)N1CCc2ccccc2C1c1ccc(F)cc1] | Compound 2503 |
| 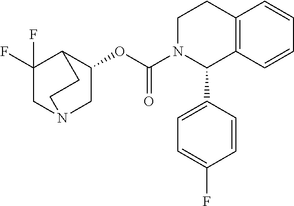 [SMILES: O=C(O[C@@H]1CN2CCC1C(F)(F)C2)N1CCc2ccccc2[C@@H]1c1ccc(F)cc1] | Compound 2504 |
| 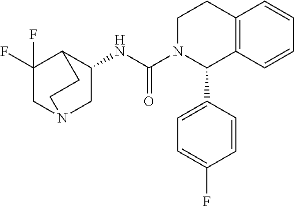 [SMILES: O=C(N[C@@H]1CN2CCC1C(F)(F)C2)N1CCc2ccccc2[C@@H]1c1ccc(F)cc1] | Compound 2505 |
| 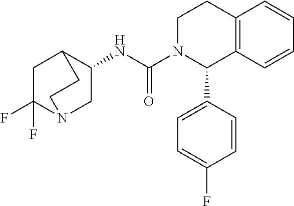 [SMILES: O=C(N[C@@H]1CN2CCC1CC2(F)F)N1CCc2ccccc2[C@@H]1c1ccc(F)cc1] | Compound 2506 |
| 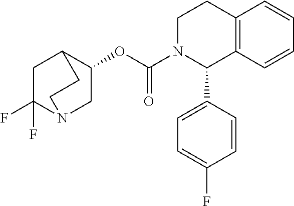 [SMILES: O=C(O[C@@H]1CN2CCC1CC2(F)F)N1CCc2ccccc2[C@@H]1c1ccc(F)cc1] | Compound 2507 |
| 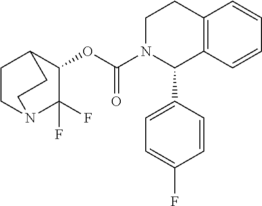 [SMILES: O=C(O[C@H]1C2CCN(CC2)C1(F)F)N1CCc2ccccc2[C@@H]1c1ccc(F)cc1] | Compound 2508 |
| 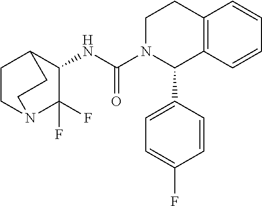 [SMILES: O=C(N[C@H]1C2CCN(CC2)C1(F)F)N1CCc2ccccc2[C@@H]1c1ccc(F)cc1] | Compound 2509 |
| 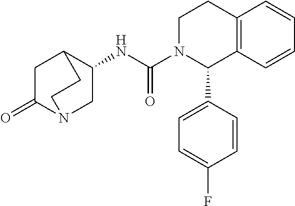 [SMILES: O=C1CC2CCN1C[C@H]2NC(=O)N1CCc2ccccc2[C@@H]1c1ccc(F)cc1] | Compound 2510 |
| 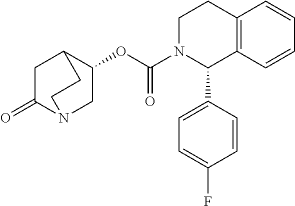 [SMILES: O=C1CC2CCN1C[C@H]2OC(=O)N1CCc2ccccc2[C@@H]1c1ccc(F)cc1] | Compound 2511 |
| 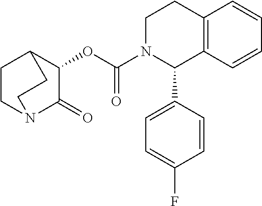 [SMILES: O=C1[C@@H](OC(=O)N2CCc3ccccc3[C@@H]2c2ccc(F)cc2)C2CCN1CC2] | Compound 2512 |
| 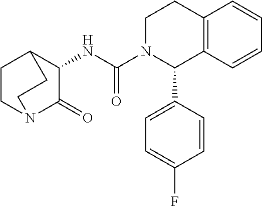 [SMILES: O=C1[C@@H](NC(=O)N2CCc3ccccc3[C@@H]2c2ccc(F)cc2)C2CCN1CC2] | Compound 2513 |
| 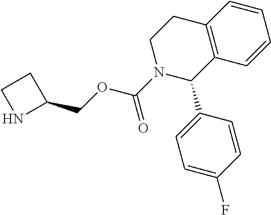 [SMILES: O=C(OC[C@@H]1CCN1)N1CCc2ccccc2[C@@H]1c1ccc(F)cc1] | Compound 2239 |
| 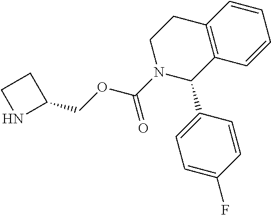 [SMILES: O=C(OC[C@H]1CCN1)N1CCc2ccccc2[C@@H]1c1ccc(F)cc1] | Compound 2240 |
| 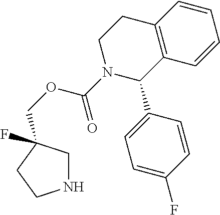 [SMILES: O=C(OC[C@@]1(F)CCNC1)N1CCc2ccccc2[C@@H]1c1ccc(F)cc1] | Compound 2255 |
| 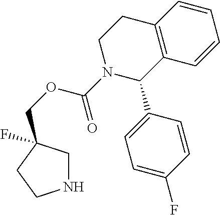 [SMILES: O=C(OC[C@]1(F)CCNC1)N1CCc2ccccc2[C@@H]1c1ccc(F)cc1] | Compound 2256 |
| 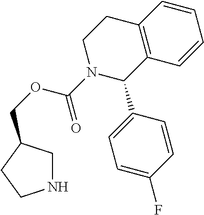 [SMILES: O=C(OC[C@@H]1CCNC1)N1CCc2ccccc2[C@@H]1c1ccc(F)cc1] | Compound 2230 |
| 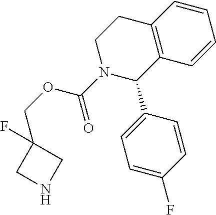 [SMILES: O=C(OCC1(F)CNC1)N1CCc2ccccc2[C@@H]1c1ccc(F)cc1] | Compound 2258 |
| 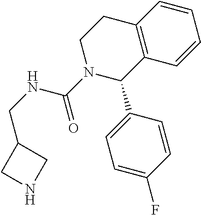 [SMILES: O=C(NCC1CNC1)N1CCc2ccccc2[C@@H]1c1ccc(F)cc1] | Compound 2257 |
| 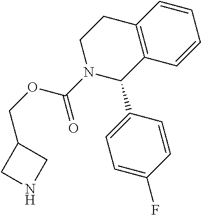 [SMILES: O=C(OCC1CNC1)N1CCc2ccccc2[C@@H]1c1ccc(F)cc1] | Compound 2246 |
| 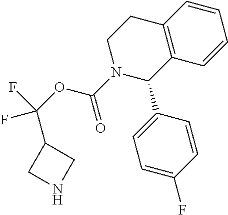 [SMILES: O=C(OC(F)(F)C1CNC1)N1CCc2ccccc2[C@@H]1c1ccc(F)cc1] | Compoudn 2514 |
| 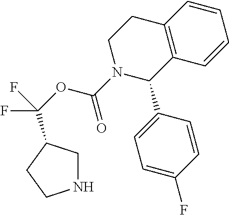 [SMILES: O=C(OC(F)(F)[C@H]1CCNC1)N1CCc2ccccc2[C@@H]1c1ccc(F)cc1] | Compound 2515 |
| 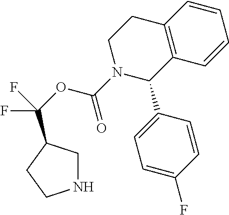 [SMILES: O=C(OC(F)(F)[C@@H]1CCNC1)N1CCc2ccccc2[C@@H]1c1ccc(F)cc1] | Compound 2516 |
| 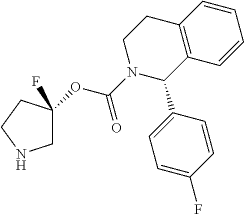 [SMILES: O=C(O[C@@]1(F)CCNC1)N1CCc2ccccc2[C@@H]1c1ccc(F)cc1] | Compound 2517 |
| 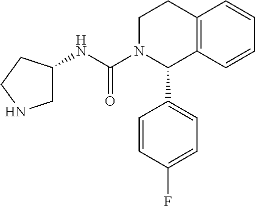 [SMILES: O=C(N[C@H]1CCNC1)N1CCc2ccccc2[C@@H]1c1ccc(F)cc1] | Compound 2222 |
| 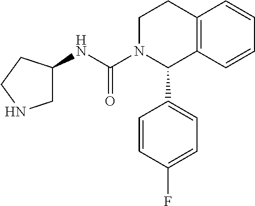 [SMILES: O=C(N[C@@H]1CCNC1)N1CCc2ccccc2[C@@H]1c1ccc(F)cc1] | Compound 2223 |
| 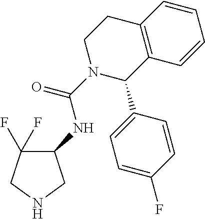 [SMILES: O=C(N[C@H]1CNCC1(F)F)N1CCc2ccccc2[C@@H]1c1ccc(F)cc1] | Compound 2243 |
| 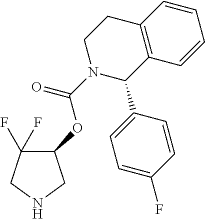 [SMILES: O=C(O[C@H]1CNCC1(F)F)N1CCc2ccccc2[C@@H]1c1ccc(F)cc1] | Compound 2241 |
| 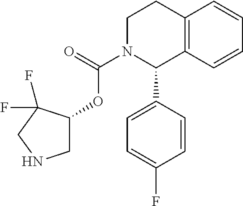 [SMILES: O=C(O[C@@H]1CNCC1(F)F)N1CCc2ccccc2[C@@H]1c1ccc(F)cc1] | Compound 2242 |
| 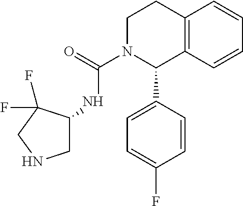 [SMILES: O=C(N[C@@H]1CNCC1(F)F)N1CCc2ccccc2[C@@H]1c1ccc(F)cc1] | Compound 2244 |
| 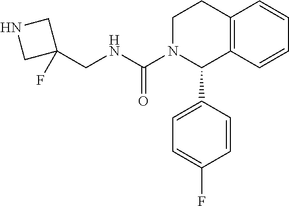 [SMILES: O=C(NCC1(F)CNC1)N1CCc2ccccc2[C@@H]1c1ccc(F)cc1] | Compound 2231 |
| 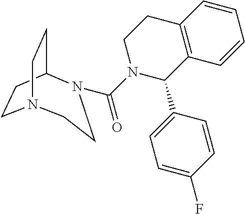 [SMILES: O=C(N1CCN2CCC1CC2)N1CCc2ccccc2[C@@H]1c1ccc(F)cc1] | Compound 2252 |
| 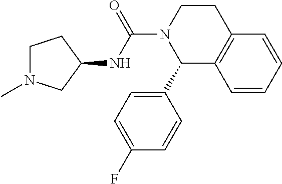 [SMILES: CN1CC[C@@H](NC(=O)N2CCc3ccccc3[C@@H]2c2ccc(F)cc2)C1] | Compound 2245 |
| 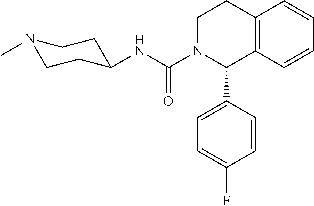 [SMILES: CN1CCC(NC(=O)N2CCc3ccccc3[C@@H]2c2ccc(F)cc2)CC1] | Compound 2238 |
| 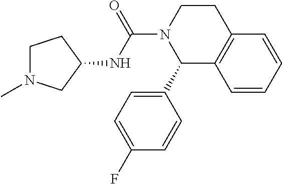 [SMILES: CN1CC[C@H](NC(=O)N2CCc3ccccc3[C@@H]2c2ccc(F)cc2)C1] | Compound 2237 |
| 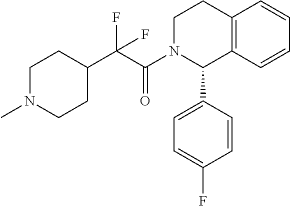 [SMILES: CN1CCC(C(F)(F)C(=O)N2CCc3ccccc3[C@@H]2c2ccc(F)cc2)CC1] | Comp;ound 2518 |
| 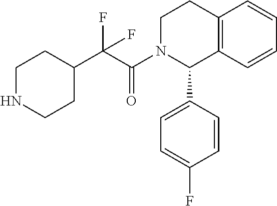 [SMILES: O=C(N1CCc2ccccc2[C@@H]1c1ccc(F)cc1)C(F)(F)C1CCNCC1] | Compound 2519 |
| 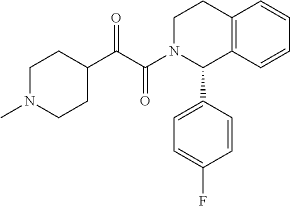 [SMILES: CN1CCC(C(=O)C(=O)N2CCc3ccccc3[C@@H]2c2ccc(F)cc2)CC1] | Compound 2520 |
| 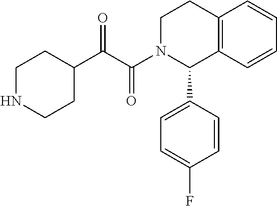 [SMILES: O=C(C(=O)N1CCc2ccccc2[C@@H]1c1ccc(F)cc1)C1CCNCC1] | Compound 2521 |
| 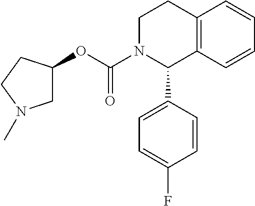 [SMILES: CN1CC[C@@H](OC(=O)N2CCc3ccccc3[C@@H]2c2ccc(F)cc2)C1] | Compound 2250 |
| 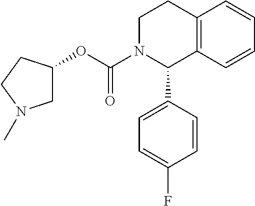 [SMILES: CN1CC[C@H](OC(=O)N2CCc3ccccc3[C@@H]2c2ccc(F)cc2)C1] | Compound 2251 |
| 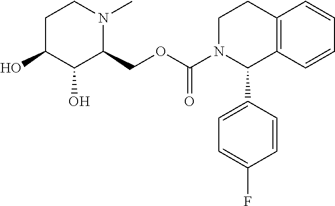 [SMILES: CN1CC[C@H](O)[C@@H](O)[C@@H]1COC(=O)N1CCc2ccccc2[C@@H]1c1ccc(F)cc1] | Compound 2304 |
| 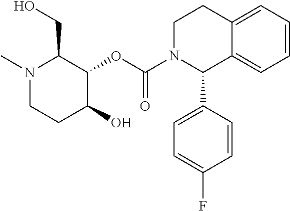 [SMILES: CN1CC[C@H](O)[C@@H](OC(=O)N2CCc3ccccc3[C@@H]2c2ccc(F)cc2)[C@@H]1CO] | Compound 2305 |
| 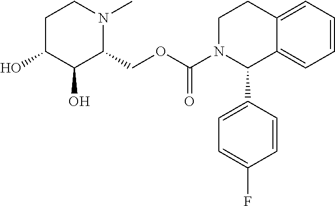 [SMILES: CN1CC[C@@H](O)[C@H](O)[C@H]1COC(=O)N1CCc2ccccc2[C@@H]1c1ccc(F)cc1] | Compound 2289 |
| 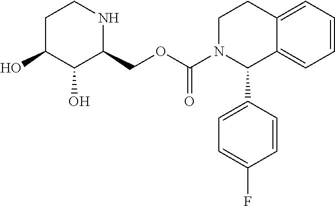 [SMILES: O=C(OC[C@@H]1NCC[C@H](O)[C@H]1O)N1CCc2ccccc2[C@@H]1c1ccc(F)cc1] | Compound 2522 |
| 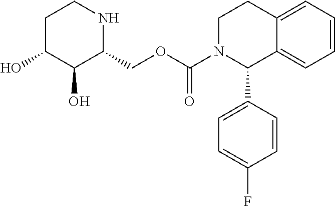 [SMILES: O=C(OC[C@H]1NCC[C@@H](O)[C@@H]1O)N1CCc2ccccc2[C@@H]1c1ccc(F)cc1] | Compound 2523 |
| 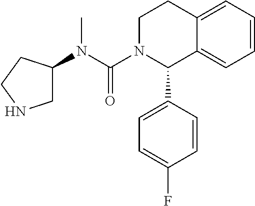 [SMILES: CN(C(=O)N1CCc2ccccc2[C@@H]1c1ccc(F)cc1)[C@@H]1CCNC1] | Compound 2253 |
| 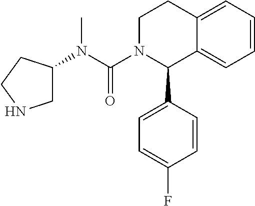 [SMILES: CN(C(=O)N1CCc2ccccc2[C@H]1c1ccc(F)cc1)[C@H]1CCNC1] | Compound 2254 |
| 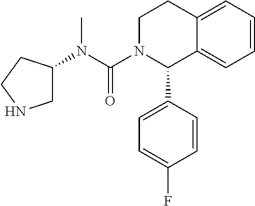 [SMILES: CN(C(=O)N1CCc2ccccc2[C@@H]1c1ccc(F)cc1)[C@H]1CCNC1] | Compound 2248 |
| 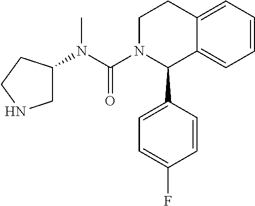 [SMILES: CN(C(=O)N1CCc2ccccc2[C@H]1c1ccc(F)cc1)[C@H]1CCNC1] | Compound 2249 |
| 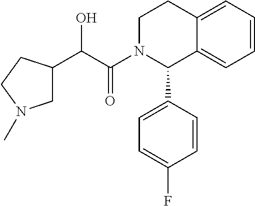 [SMILES: CN1CCC(C(O)C(=O)N2CCc3ccccc3[C@@H]2c2ccc(F)cc2)C1] | Compound 2267 |
| 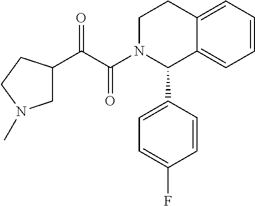 [SMILES: CN1CCC(C(=O)C(=O)N2CCc3ccccc3[C@@H]2c2ccc(F)cc2)C1] | Compound 2274 |
| 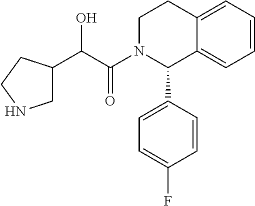 [SMILES: O=C(C(O)C1CCNC1)N1CCc2ccccc2[C@@H]1c1ccc(F)cc1] | Compound 2524 |
| 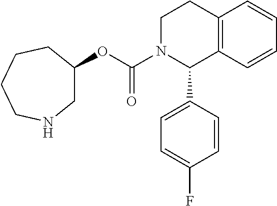 [SMILES: O=C(O[C@@H]1CCCCNC1)N1CCc2ccccc2[C@@H]1c1ccc(F)cc1] | Compound 2525 |
| 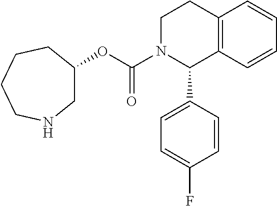 [SMILES: O=C(O[C@H]1CCCCNC1)N1CCc2ccccc2[C@@H]1c1ccc(F)cc1] | Compound 2526 |
| 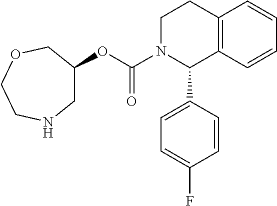 [SMILES: O=C(O[C@H]1CNCCOC1)N1CCc2ccccc2[C@@H]1c1ccc(F)cc1] | Compound 2263 |
| 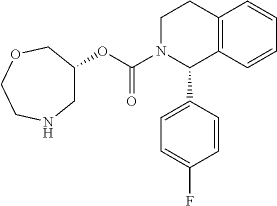 [SMILES: O=C(O[C@@H]1CNCCOC1)N1CCc2ccccc2[C@@H]1c1ccc(F)cc1] | Compound 2260 |
| 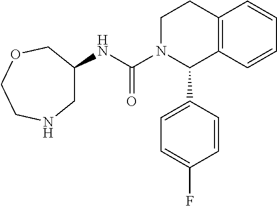 [SMILES: O=C(N[C@H]1CNCCOC1)N1CCc2ccccc2[C@@H]1c1ccc(F)cc1] | Compound 2275 |
| 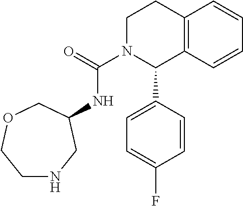 [SMILES: O=C(N[C@H]1CNCCOC1)N1CCc2ccccc2[C@@H]1c1ccc(F)cc1] | Compound 2276 |
| 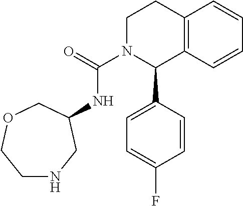 [SMILES: O=C(N[C@H]1CNCCOC1)N1CCc2ccccc2[C@H]1c1ccc(F)cc1] | Compound 2278 |
| 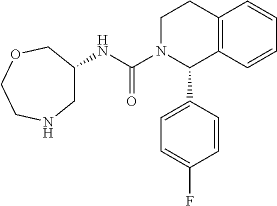 [SMILES: O=C(N[C@@H]1CNCCOC1)N1CCc2ccccc2[C@@H]1c1ccc(F)cc1] | Compound 2277 |
| 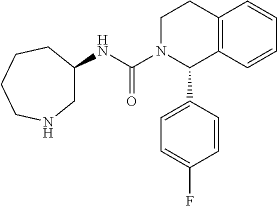 [SMILES: O=C(N[C@@H]1CCCCNC1)N1CCc2ccccc2[C@@H]1c1ccc(F)cc1] | Compound 2527 |
| 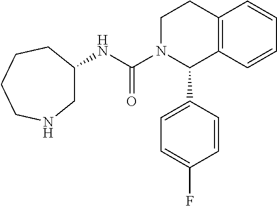 [SMILES: O=C(N[C@H]1CCCCNC1)N1CCc2ccccc2[C@@H]1c1ccc(F)cc1] | Compound 2528 |
| 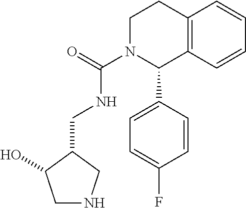 | Compound 2308 |
| 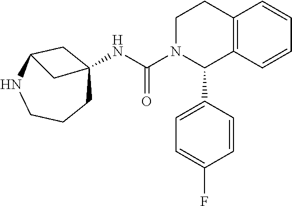 [SMILES: O=C(N[C@]12CCCN[C@H](C1)C2)N1CCc2ccccc2[C@@H]1c1ccc(F)cc1] | Compound 2529 |
| 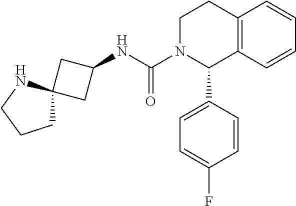 [SMILES: O=C(N[C@H]1C[C@]2(CCCN2)C1)N1CCc2ccccc2[C@@H]1c1ccc(F)cc1] | Compound 2530 |
| 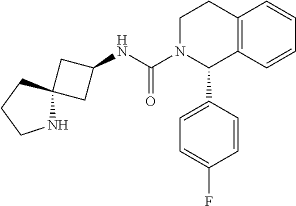 [SMILES: O=C(N[C@H]1C[C@@]2(CCCN2)C1)N1CCc2ccccc2[C@@H]1c1ccc(F)cc1] | Compound 2531 |
| 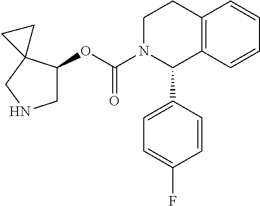 [SMILES: O=C(O[C@H]1CNCC12CC2)N1CCc2ccccc2[C@@H]1c1ccc(F)cc1] | Compound 2309 |
| 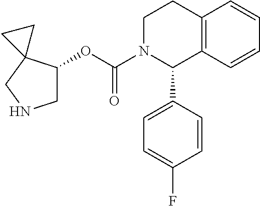 [SMILES: O=C(O[C@@H]1CNCC12CC2)N1CCc2ccccc2[C@@H]1c1ccc(F)cc1] | Compound 2310 |
| 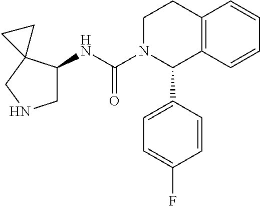 [SMILES: O=C(N[C@H]1CNCC12CC2)N1CCc2ccccc2[C@@H]1c1ccc(F)cc1] | Compound 2280 |
| 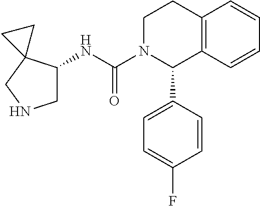 [SMILES: O=C(N[C@@H]1CNCC12CC2)N1CCc2ccccc2[C@@H]1c1ccc(F)cc1] | Compound 2281 |
| 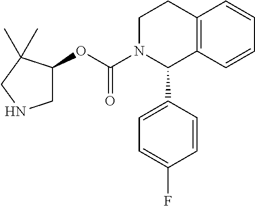 [SMILES: CC1(C)CNC[C@@H]1OC(=O)N1CCc2ccccc2[C@@H]1c1ccc(F)cc1] | Compound 2291 |
| 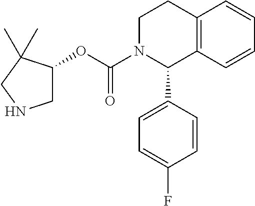 [SMILES: CC1(C)CNC[C@H]1OC(=O)N1CCc2ccccc2[C@@H]1c1ccc(F)cc1] | Compound 2292 |
| 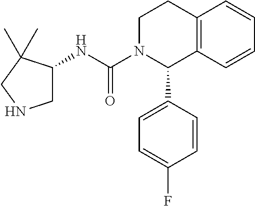 [SMILES: CC1(C)CNC[C@H]1NC(=O)N1CCc2ccccc2[C@@H]1c1ccc(F)cc1] | Compound 2273 |
| 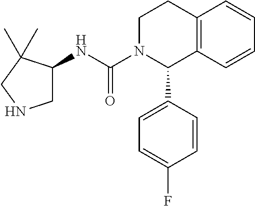 [SMILES: CC1(C)CNC[C@@H]1NC(=O)N1CCc2ccccc2[C@@H]1c1ccc(F)cc1] | Compound 2268 |
| 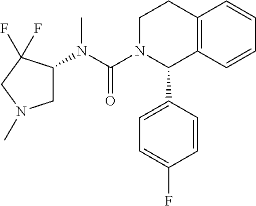 [SMILES: CN1C[C@@H](N(C)C(=O)N2CCc3ccccc3[C@@H]2c2ccc(F)cc2)C(F)(F)C1] | Compound 2298 |
| 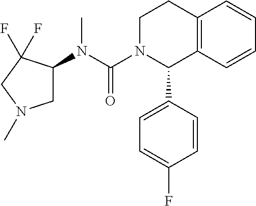 [SMILES: CN1C[C@H](N(C)C(=O)N2CCc3ccccc3[C@@H]2c2ccc(F)cc2)C(F)(F)C1] | Compound 2299 |
| 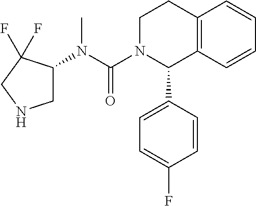 [SMILES: CN(C(=O)N1CCc2ccccc2[C@@H]1c1ccc(F)cc1)[C@@H]1CNCC1(F)F] | Compound 2296 |
| 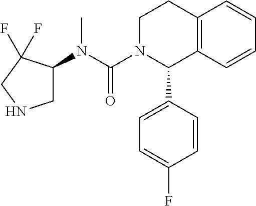 [SMILES: CN(C(=O)N1CCc2ccccc2[C@@H]1c1ccc(F)cc1)[C@H]1CNCC1(F)F] | Compound 2297 |
| 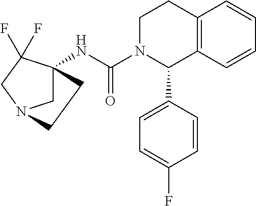 [SMILES: O=C(N[C@@]12CC[N@@](CC1(F)F)C2)N1CCc2ccccc2[C@@H]1c1ccc(F)cc1] | Compound 2532 |
| 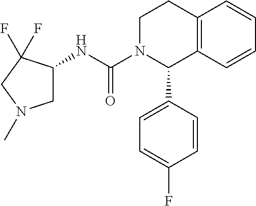 [SMILES: CN1C[C@@H](NC(=O)N2CCc3ccccc3[C@@H]2c2ccc(F)cc2)C(F)(F)C1] | Compound 2293 |
| 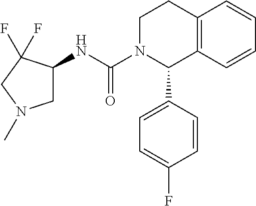 [SMILES: CN1C[C@H](NC(=O)N2CCc3ccccc3[C@@H]2c2ccc(F)cc2)C(F)(F)C1] | Compound 2294 |
| 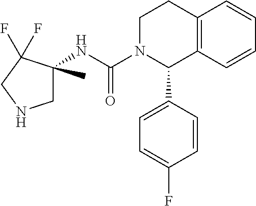 [SMILES: C[C@@]1(NC(=O)N2CCc3ccccc3[C@@H]2c2ccc(F)cc2)CNCC1(F)F] | Compound 2533 |
| 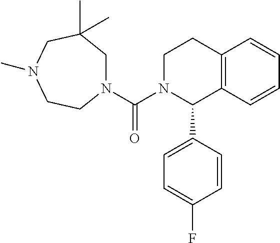 [SMILES: CN1CCN(C(=O)N2CCc3ccccc3[C@@H]2c2ccc(F)cc2)CC(C)(C)C1] | Compound 2534 |
| 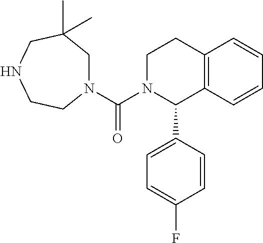 [SMILES: CC1(C)CNCCN(C(=O)N2CCc3ccccc3[C@@H]2c2ccc(F)cc2)C1] | Compound 2535 |
| 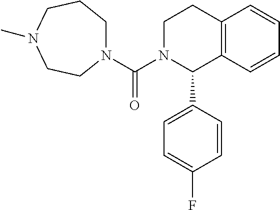 [SMILES: CN1CCCN(C(=O)N2CCc3ccccc3[C@@H]2c2ccc(F)cc2)CC1] | Compound 2269 |
| 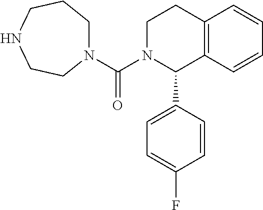 [SMILES: O=C(N1CCCNCC1)N1CCc2ccccc2[C@@H]1c1ccc(F)cc1] | Compound 2264 |
| 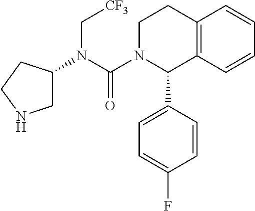 [SMILES: O=C(N(CC(F)(F)F)[C@H]1CCNC1)N1CCc2ccccc2[C@@H]1c1ccc(F)cc1] | Compound 2536 |
| 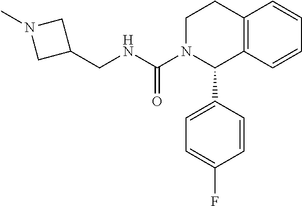 [SMILES: CN1CC(CNC(=O)N2CCc3ccccc3[C@@H]2c2ccc(F)cc2)C1] | Compound 2282 |
| 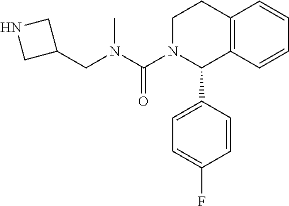 [SMILES: CN(CC1CNC1)C(=O)N1CCc2ccccc2[C@@H]1c1ccc(F)cc1] | Compound 2283 |
| 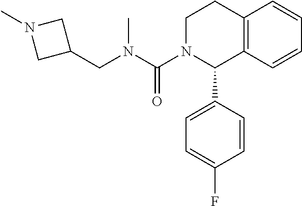 [SMILES: CN1CC(CN(C)C(=O)N2CCc3ccccc3[C@@H]2c2ccc(F)cc2)C1] | Compound 2537 |
| 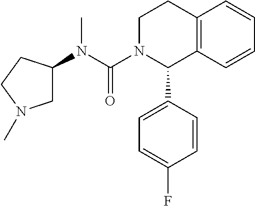 [SMILES: CN1CC[C@@H](N(C)C(=O)N2CCc3ccccc3[C@@H]2c2ccc(F)cc2)C1] | Compound 2265 |
| 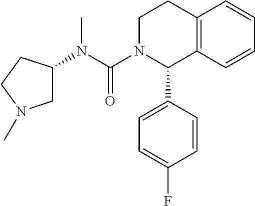 [SMILES: CN1CC[C@H](N(C)C(=O)N2CCc3ccccc3[C@@H]2c2ccc(F)cc2)C1] | Compound 2270 |
| 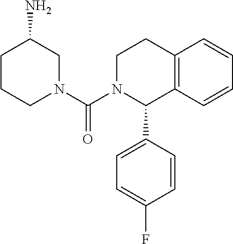 [SMILES: N[C@H]1CCCN(C(=O)N2CCc3ccccc3[C@@H]2c2ccc(F)cc2)C1] | Compound 2538 |
| 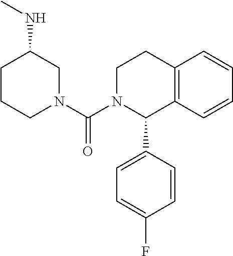 [SMILES: CN[C@H]1CCCN(C(=O)N2CCc3ccccc3[C@@H]2c2ccc(F)cc2)C1] | Compound 2539 |
| 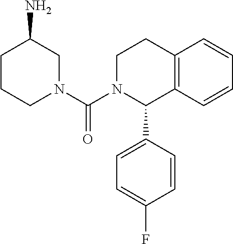 [SMILES: N[C@@H]1CCCN(C(=O)N2CCc3ccccc3[C@@H]2c2ccc(F)cc2)C1] | Compound 2540 |
| 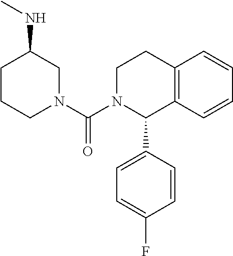 [SMILES: CN[C@@H]1CCCN(C(=O)N2CCc3ccccc3[C@@H]2c2ccc(F)cc2)C1] | Compound 2541 |
| 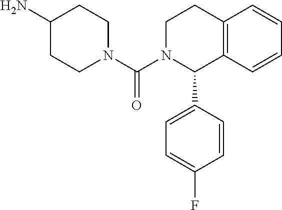 [SMILES: NC1CCN(C(=O)N2CCc3ccccc3[C@@H]2c2ccc(F)cc2)CC1] | Compound 2542 |
| 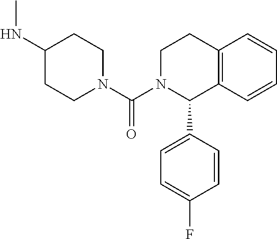 [SMILES: CNC1CCN(C(=O)N2CCc3ccccc3[C@@H]2c2ccc(F)cc2)CC1] | Compound 2543 |
| 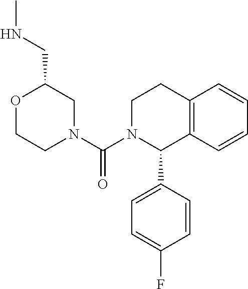 [SMILES: CNC[C@@H]1CN(C(=O)N2CCc3ccccc3[C@@H]2c2ccc(F)cc2)CCO1] | Compound 2306 |
| 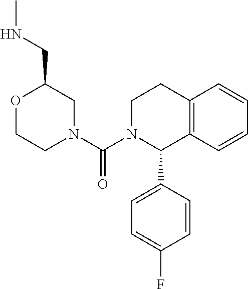 [SMILES: CNC[C@H]1CN(C(=O)N2CCc3ccccc3[C@@H]2c2ccc(F)cc2)CCO1] | Compound 2307 |
| 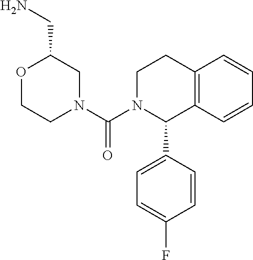 [SMILES: NC[C@@H]1CN(C(=O)N2CCc3ccccc3[C@@H]2c2ccc(F)cc2)CCO1] | Compound 2302 |
| 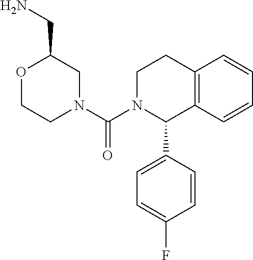 [SMILES: NC[C@H]1CN(C(=O)N2CCc3ccccc3[C@@H]2c2ccc(F)cc2)CCO1] | Compound 2303 |
| 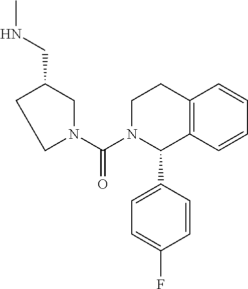 [SMILES: CNC[C@H]1CCN(C(=O)N2CCc3ccccc3[C@@H]2c2ccc(F)cc2)C1] | Compound 2544 |
| 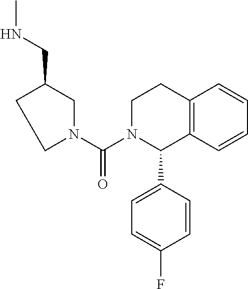 [SMILES: CNC[C@@H]1CCN(C(=O)N2CCc3ccccc3[C@@H]2c2ccc(F)cc2)C1] | Compound 2545 |
| 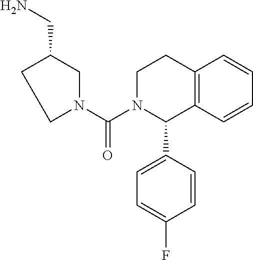 [SMILES: NC[C@H]1CCN(C(=O)N2CCc3ccccc3[C@@H]2c2ccc(F)cc2)C1] | Compound 2288 |
| 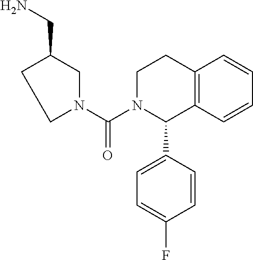 [SMILES: NC[C@@H]1CCN(C(=O)N2CCc3ccccc3[C@@H]2c2ccc(F)cc2)C1] | Compound 2279 |
| 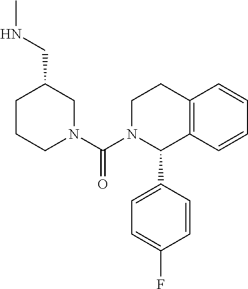 [SMILES: CNC[C@H]1CCCN(C(=O)N2CCc3ccccc3[C@@H]2c2ccc(F)cc2)C1] | Compound 2546 |
| 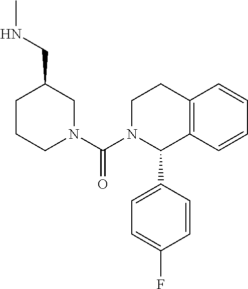 [SMILES: CNC[C@@H]1CCCN(C(=O)N2CCc3ccccc3[C@@H]2c2ccc(F)cc2)C1] | Compound 2547 |
| 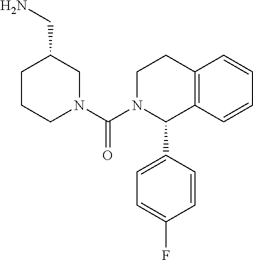 [SMILES: NC[C@H]1CCCN(C(=O)N2CCc3ccccc3[C@@H]2c2ccc(F)cc2)C1] | Compound 2548 |
| 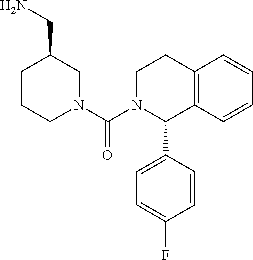 [SMILES: NC[C@@H]1CCCN(C(=O)N2CCc3ccccc3[C@@H]2c2ccc(F)cc2)C1] | Compound 2549 |
| 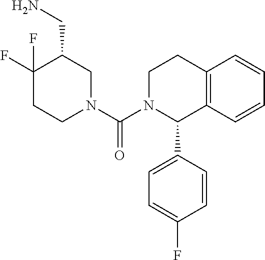 [SMILES: NC[C@@H]1CN(C(=O)N2CCc3ccccc3[C@@H]2c2ccc(F)cc2)CCC1(F)F] | Compound 2550 |
| 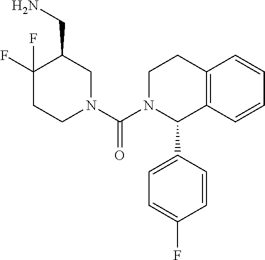 [SMILES: NC[C@H]1CN(C(=O)N2CCc3ccccc3[C@@H]2c2ccc(F)cc2)CCC1(F)F] | Compound 2551 |
| 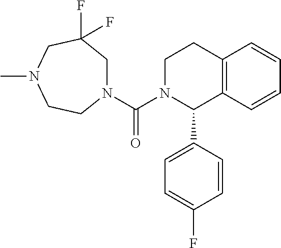 [SMILES: CN1CCN(C(=O)N2CCc3ccccc3[C@@H]2c2ccc(F)cc2)CC(F)(F)C1] | Compound 2261 |
| 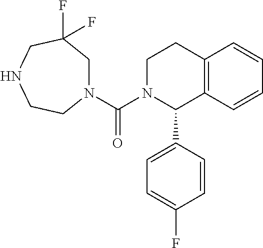 [SMILES: O=C(N1CCNCC(F)(F)C1)N1CCc2ccccc2[C@@H]1c1ccc(F)cc1] | Compound 2262 |
| 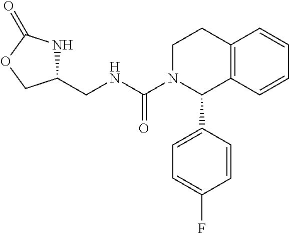 [SMILES: O=C1N[C@H](CNC(=O)N2CCc3ccccc3[C@@H]2c2ccc(F)cc2)CO1] | Compound 2259 |
| 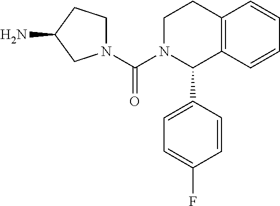 [SMILES: N[C@H]1CCN(C(=O)N2CCc3ccccc3[C@@H]2c2ccc(F)cc2)C1] | Compound 2284 |
| 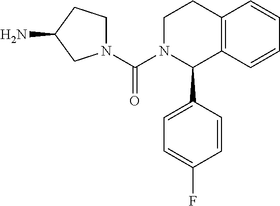 [SMILES: N[C@H]1CCN(C(=O)N2CCc3ccccc3[C@H]2c2ccc(F)cc2)C1] | Compound 2285 |
| 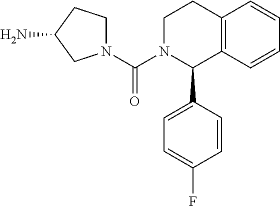 [SMILES: N[C@@H]1CCN(C(=O)N2CCc3ccccc3[C@H]2c2ccc(F)cc2)C1] | Compound 2286 |
| 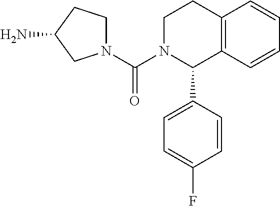 [SMILES: N[C@@H]1CCN(C(=O)N2CCc3ccccc3[C@@H]2c2ccc(F)cc2)C1] | Compound 2287 |
| 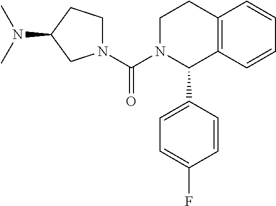 [SMILES: CN(C)[C@H]1CCN(C(=O)N2CCc3ccccc3[C@@H]2c2ccc(F)cc2)C1] | Compound 2311 |
| 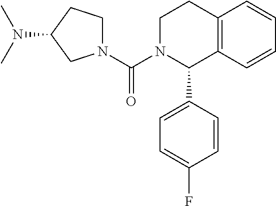 [SMILES: CN(C)[C@@H]1CCN(C(=O)N2CCc3ccccc3[C@@H]2c2ccc(F)cc2)C1] | Compound 2312 |
| 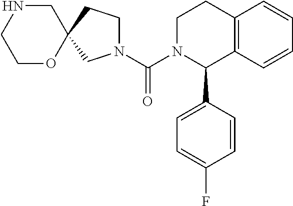 [SMILES: O=C(N1CC[C@@]2(CNCCO2)C1)N1CCc2ccccc2[C@H]1c1ccc(F)cc1] | Compound 2323 |
| 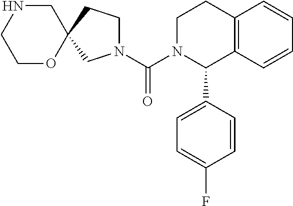 [SMILES: O=C(N1CC[C@@]2(CNCCO2)C1)N1CCc2ccccc2[C@@H]1c1ccc(F)cc1] | Compound 2324 |
| 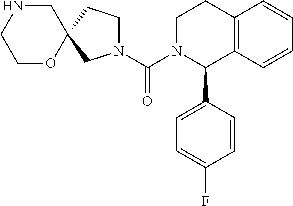 [SMILES: O=C(N1CC[C@]2(CNCCO2)C1)N1CCc2ccccc2[C@H]1c1ccc(F)cc1] | Compound 2325 |
| 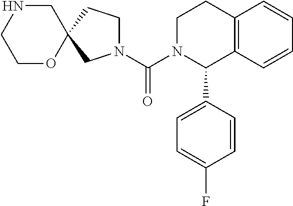 [SMILES: O=C(N1CC[C@]2(CNCCO2)C1)N1CCc2ccccc2[C@@H]1c1ccc(F)cc1] | Compound 2326 |
| 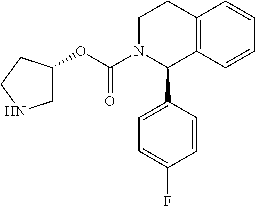 [SMILES: O=C(O[C@H]1CCNC1)N1CCc2ccccc2[C@H]1c1ccc(F)cc1] | Compound 2272 |
| 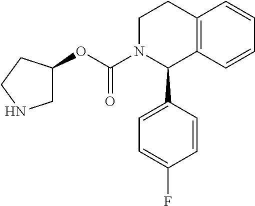 [SMILES: O=C(O[C@@H]1CCNC1)N1CCc2ccccc2[C@H]1c1ccc(F)cc1] | Compound 2266 |
| 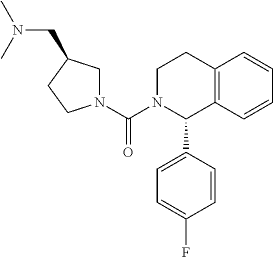 [SMILES: CN(C)C[C@@H]1CCN(C(=O)N2CCc3ccccc3[C@@H]2c2ccc(F)cc2)C1] | Compound 2313 |
| 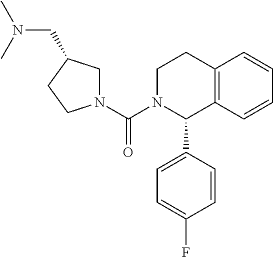 [SMILES: CN(C)C[C@H]1CCN(C(=O)N2CCc3ccccc3[C@@H]2c2ccc(F)cc2)C1] | Compound 2314 |
| 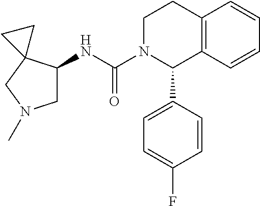 [SMILES: CN1C[C@H](NC(=O)N2CCc3ccccc3[C@@H]2c2ccc(F)cc2)C2(CC2)C1] | Compound 2315 |
| 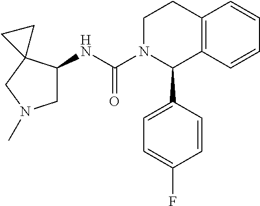 [SMILES: CN1C[C@H](NC(=O)N2CCc3ccccc3[C@H]2c2ccc(F)cc2)C2(CC2)C1] | Compound 2316 |
| 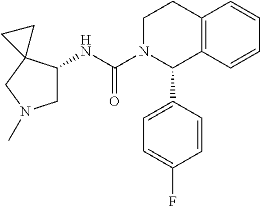 [SMILES: CN1C[C@@H](NC(=O)N2CCc3ccccc3[C@@H]2c2ccc(F)cc2)C2(CC2)C1] | Compound 2317 |
| 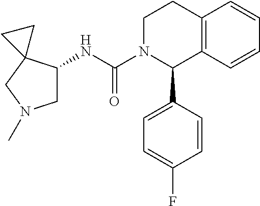 [SMILES: CN1C[C@@H](NC(=O)N2CCc3ccccc3[C@H]2c2ccc(F)cc2)C2(CC2)C1] | Compound 2318 |
| 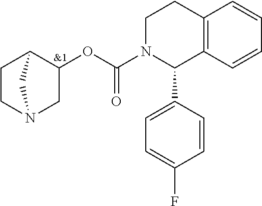 [SMILES: O=C(OC1C[N@@]2CC[C@@H]1C2)N1CCc2ccccc2[C@@H]1c1ccc(F)cc1] | Compound 2203 |
| 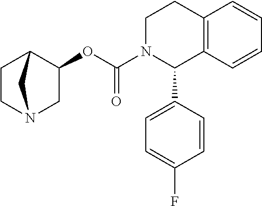 [SMILES: O=C(O[C@H]1C[N@]2CC[C@H]1C2)N1CCc2ccccc2[C@@H]1c1ccc(F)cc1] | Compound 2204 |
| 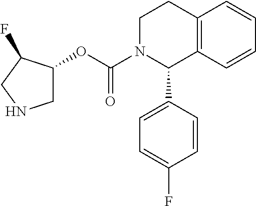 [SMILES: O=C(O[C@@H]1CNC[C@H]1F)N1CCc2ccccc2[C@@H]1c1ccc(F)cc1] | Compound 2349 |
| 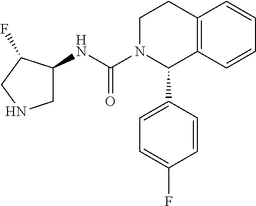 [SMILES: O=C(N[C@H]1CNC[C@@H]1F)N1CCc2ccccc2[C@@H]1c1ccc(F)cc1] | Compound 2351 |
| 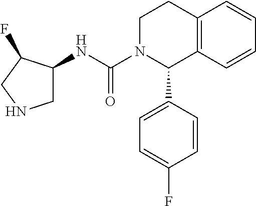 [SMILES: O=C(N[C@H]1CNC[C@H]1F)N1CCc2ccccc2[C@@H]1c1ccc(F)cc1] | Compound 2352 |
| 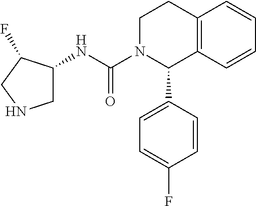 [SMILES: O=C(N[C@@H]1CNC[C@@H]1F)N1CCc2ccccc2[C@@H]1c1ccc(F)cc1] | Compound 2353 |
| 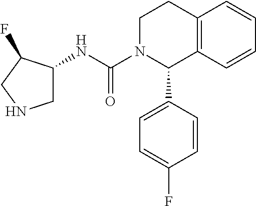 [SMILES: O=C(N[C@@H]1CNC[C@H]1F)N1CCc2ccccc2[C@@H]1c1ccc(F)cc1] | Compound 2360 |
| 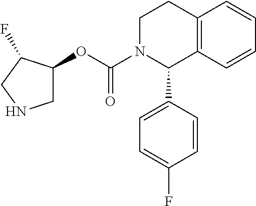 [SMILES: O=C(O[C@H]1CNC[C@@H]1F)N1CCc2ccccc2[C@@H]1c1ccc(F)cc1] | Compound 2361 |
| 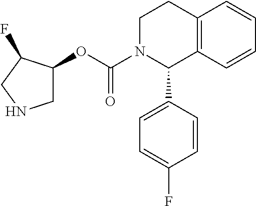 [SMILES: O=C(O[C@H]1CNC[C@H]1F)N1CCc2ccccc2[C@@H]1c1ccc(F)cc1] | Compound 2362 |
| 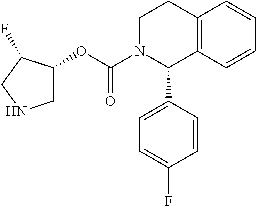 [SMILES: O=C(O[C@@H]1CNC[C@@H]1F)N1CCc2ccccc2[C@@H]1c1ccc(F)cc1] | Compound 2363 |
| 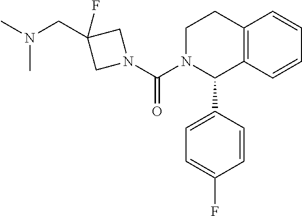 [SMILES: CN(C)CC1(F)CN(C(=O)N2CCc3ccccc3[C@@H]2c2ccc(F)cc2)C1] | Compound 2364 |
| 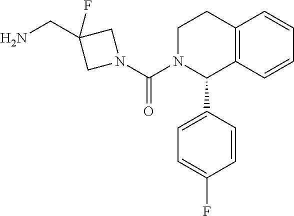 [SMILES: NCC1(F)CN(C(=O)N2CCc3ccccc3[C@@H]2c2ccc(F)cc2)C1] | Compound 2367 |
| 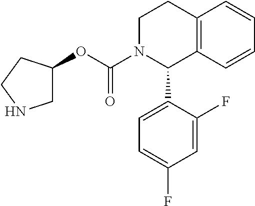 [SMILES: O=C(O[C@@H]1CCNC1)N1CCc2ccccc2[C@@H]1c1ccc(F)cc1F] | Compound 2368 |
| 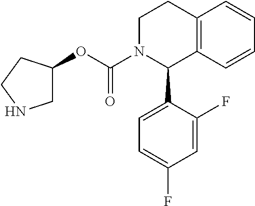 [SMILES: O=C(O[C@@H]1CCNC1)N1CCc2ccccc2[C@H]1c1ccc(F)cc1F] | Compound 2369 |
| 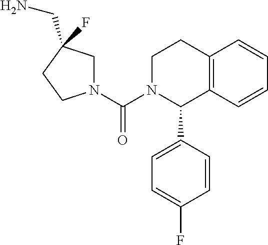 [SMILES: NC[C@@]1(F)CCN(C(=O)N2CCc3ccccc3[C@@H]2c2ccc(F)cc2)C1] | Compound 2370 |
| 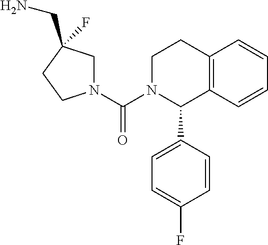 [SMILES: NC[C@]1(F)CCN(C(=O)N2CCc3ccccc3[C@@H]2c2ccc(F)cc2)C1] | Compound 2371 |
| 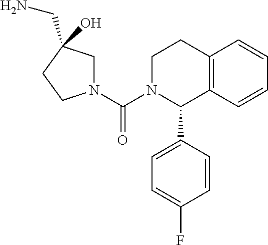 [SMILES: NC[C@@]1(O)CCN(C(=O)N2CCc3ccccc3[C@@H]2c2ccc(F)cc2)C1] | Compound 2372 |
| 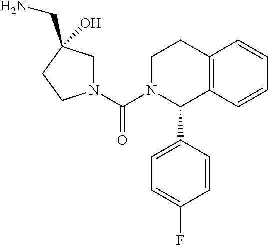 [SMILES: NC[C@]1(O)CCN(C(=O)N2CCc3ccccc3[C@@H]2c2ccc(F)cc2)C1] | Compound 2373 |

As used herein, the term “alkyl” refers to straight chained and branched saturated hydrocarbon groups containing one to thirty carbon atoms, for example, one to twenty carbon atoms, or one to ten carbon atoms. The term CO means the alkyl group has “n” carbon atoms. For example, C₄ alkyl refers to an alkyl group that has 4 carbon atoms. C₁-C₆ alkyl refers to an alkyl group having a number of carbon atoms encompassing the entire range (e.g., 1 to 6 carbon atoms), as well as all subgroups (e.g., 1-6, 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), t-butyl (1,1-dimethylethyl), and 3-methylpentyl. Unless otherwise indicated, an alkyl group can be an unsubstituted alkyl group or a substituted alkyl group.

The term “alkylene” used herein refers to an alkyl group having a substituent. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group.

As used herein, the term “alkoxy” or “alkoxyl” as used herein refers to a “—O-alkyl” group. The alkoxy or alkoxyl group can be unsubstituted or substituted.

As used herein, the term “halo” refers to an atom selected from Group 17 of the periodic table, e.g., fluorine, chlorine, bromine, or iodine. Moieties described herein can be substituted with a halo group. For example, a halo-substituted aryl moiety can be e.g., a fluorophenyl moiety.

As used herein, the term “cycloalkyl” refers to an aliphatic cyclic hydrocarbon group containing four to ten carbon atoms (e.g., 4, 5, 6, 7, 8, 9, or 10 carbon atoms). The term Cₙ means the cycloalkyl group has “n” carbon atoms. For example, C₅ cycloalkyl refers to a cycloalkyl group that has 5 carbon atoms in the ring. C₆-C₁₀ cycloalkyl refers to cycloalkyl groups having a number of carbon atoms encompassing the entire range (e.g., 6 to 10 carbon atoms), as well as all subgroups (e.g., 6-7, 6-8, 7-8, 6-9, 6, 7, 8, 9, and 10 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group. When a cycloalkyl group is fused to another cycloalkyl group, then each of the cycloalkyl groups can contain three to ten carbon atoms unless specified otherwise. Unless otherwise indicated, a cycloalkyl group can be unsubstituted or substituted.

As used herein, the term “heterocycle” is defined similarly as cycloalkyl, except the ring contains one to three heteroatoms independently selected from oxygen, nitrogen, and sulfur. In particular, the term “heterocycle” refers to a ring containing a total of three to ten atoms (e.g., four to ten), of which 1, 2, 3 or three of those atoms are heteroatoms independently selected from the group consisting of oxygen, nitrogen, and sulfur, and the remaining atoms in the ring are carbon atoms. Nonlimiting examples of heterocycle groups include azetidine, piperdine, piperazine, pyrazolidine, tetrahydrofuran, tetrahydropyran, dihydrofuran, morpholine, quinuclidine, and the like. Heterocycle groups can be saturated or partially unsaturated ring systems optionally substituted with, for example, one to three groups, such as C₁₋₆alkyl. The heterocycle groups described herein can be isolated or fused to another heterocycle group and/or a cycloalkyl group. In particular, the heterocycles described herein can have a fused, bridged, or spiro structure. When a heterocycle group is fused to another heterocycle group, then each of the heterocycle groups can contain three to ten total ring atoms, and one to three heteroatoms. Unless otherwise indicated, a heterocycle group can be unsubstituted or substituted.

As used herein, the term “aryl” refers to an aromatic group, such as phenyl. Aryl groups can be e.g., monocyclic or polycyclic. Unless otherwise indicated, an aryl group can be unsubstituted or substituted with one or more, and in particular one group selected from, for example, alkoxy and alkoxyalkyl. Aryl groups can be isolated (e.g., phenyl) or fused to another aryl group (e.g., naphthyl, anthracenyl), a cycloalkyl group (e.g. tetraydronaphthyl), a heterocycloalkyl group, and/or a heteroaryl group. Exemplary aryl groups include, but are not limited to, phenyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic aromatic ring having 5 to 6 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, alkoxy. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl (e.g., propargyl), heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

Compounds of the present disclosure can exist in particular geometric or stereoisomeric forms having one or more asymmetric carbon atoms. The present disclosure contemplates such forms, including cis- and trans-isomers, R- and S-enantiomers, diastereomers, racemic mixtures thereof, and other mixtures thereof, as falling within the scope of the disclosed compounds. Additional asymmetric carbon atoms may be present in substituents such as alkyl groups. All such isomers, as well as mixtures thereof, are intended for inclusion herein.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Pharmaceutical Formulations, Dosing, and Routes of Administration

Further provided are pharmaceutical formulations comprising a compound as described herein or pharmaceutically acceptable salt thereof, and a pharmaceutically acceptable excipient.

The compounds described herein can be administered to a subject in a therapeutically effective amount, alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to affect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, for example, from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by physico-chemical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, Pa., pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus, the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

Compounds of Formula I can affect cells to increase secretion of progranulin. Solifenacin is a drug currently used for urinary incontinence. It has been found that this compound also causes the secretion of progranulin from mouse BV2 cells. As such, compounds of Formula I can be useful in treating disorders associated with aberrant (e.g., reduced) progranulin secretion or activity.

Specifically contemplated are methods of using a therapeutically effective amount of a compound disclosed herein to modulate progranulin (e.g., to increase secretion of progranulin), for use as a therapeutic in a subject. As used herein, the term “therapeutically effective amount” means an amount of a compound or combination of therapeutically active compounds (e.g., a progranulin modulator or combination of modulators) that ameliorates, attenuates or eliminates one or more symptoms of a particular disease or condition (e.g., progranulin- or granulin-associated), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

Contemplated disorders associated with aberrant progranulin activity include Alzheimer's disease (AD), Parkinson's disease (PD) and PD-related disorders, Amytrophic lateral sclerosis (ALS), Frontotemperal lobe dementia (FTLD), Lewy body dementia (LBD), Prion disease, Motor neurone diseases (MND), Huntington's disease (HD), Spinocerebellar ataxia (SCA), Spinal muscular atrophy (SMA) and other neurodegenerative diseases. Other disorders contemplated include lysosomal dys- or misfunction disorders, such lysosomal storage diseases (e.g., Paget's disease, Gaucher's disease, Nieman's Pick disease, Tay-Sachs Disease, Fabry Disease, Pompes disease, and Naso-Hakula disease). Other diseases contemplated include those associated with inclusions and/or misfunction of C9orf72, TDP-43, FUS, UBQLN2, VCP, CHMP28, and/or MAPT. Other diseases include acute neurological disorders such as stroke, cerebral hemorrhage, traumatic brain injury and other head traumas as well as diseases of the brain such as glioblastoma and neuroblastomas.

### Synthesis of Compounds Disclosed Herein

Compounds can be synthesized in using typical synthetic chemistry techniques using commercially available starting materials, compounds known in the literature, or from readily prepared intermediates, by employing standard synthetic methods and procedures either known to those of skill in the art, or in light of the teachings herein. Generally, the synthesis of the disclosed compounds can be achieved following similar synthesis as detailed in WO 96/20194 (Yamanouchi Pharma) and WO 2012/001481 (Aurobindo).

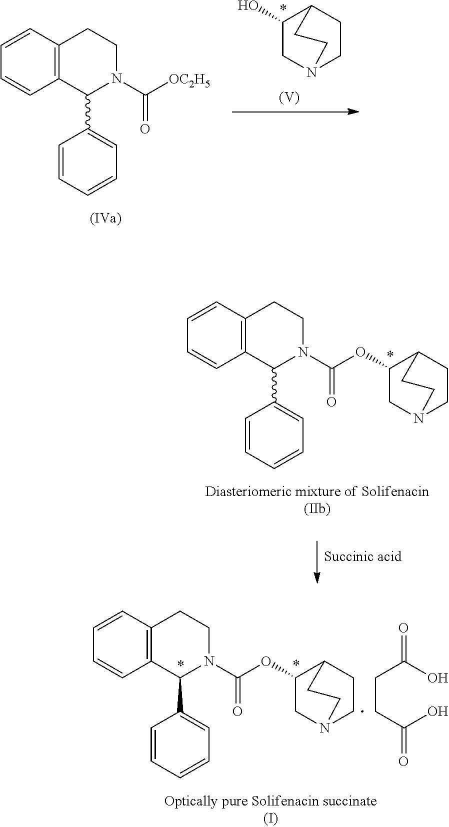

### EXAMPLES

### General Methods

All ¹HNMR experiments were run in Bruker Avance III 400, at 25° C.

### Analytical Methods:

All CP Analytical-SFC experiments were run on SFC Method Station (Thar, Waters), Column temperature: 40° C., Mobile phase: CO₂/Methanol (0.2% Methanol Ammonia)=Flow: 4.0 ml/min, Back Pressure: 120 Bar, Detection wavelength: 214 nm;

### Preparative Methods:

All CP Preparative-SFC experiments were run on SFC-80 (Thar, Waters), Column temperature: 35° C., Mobile phase (example): CO₂/Methanol (0.2% Methanol Ammonia)=Flow rate: 80 g/min, Back pressure: 100 bar, Detection wavelength: 214 nm.

Preparative CP Method B: Acidic reversed phase MPLC: Instrument type: Reveleris™ prep MPLC; Column: Phenomenex LUNA C18(3) (150×25 mm, 10p); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.

### LCMS Experiments:

All CP LCMS experiments were run on an Agilent 1200 system, with a column temperature of 40° C., monitoring UV absorption at 214 nm and scanning a mass range from 100-1000. Individual conditions varied slightly as described in the methods below:

LCMS CP Method A: Column: ZORBAX SB-C18 3.0×50 mm, 3.5 μm; Mobile Phase: A: Water (0.1% v/v TFA), B: ACN (0.1% v/v TFA); Gradient: 5% B increasing to 95% B over 1.3 min, stopping at 3 min. Flow Rate: 1.8 mL/min

LCMS CP Method A1: Column: XBridge SB-C18 3.0×50 mm, 3.5 μm; Mobile Phase: A: Water (0.01% v/v TFA), B: ACN (0.01% v/v TFA); Gradient: 5% B increasing to 95% B over 1.3 min, stopping at 3 min. Flow Rate: 2.0 mL/min

LCMS CP Method A2: Column: SunFire-C18 3.0×50 mm, 3.5 μm; Mobile Phase: A: Water (0.01% v/v TFA), B: ACN (0.01% v/v TFA); Gradient: 5% B increasing to 95% B over 1.3 min, stopping at 3 min. Flow Rate: 2.0 mL/min

LCMS CP Method B: Column: XBridge C18 50×4.6 mm, 3.5 μm; Mobile Phase: A: Water (0.1% v/v TFA), B: ACN (0.1% v/v TFA); Gradient: 5% B increasing to 95% B over 1.2 min, stopping at 3 min. Flow Rate: 2.0 mL/min

LCMS CP Method C: Column: XBridge SB-C18 3.0×50 mm, 3.5 μm; Mobile Phase: A: Water (10 mM NH₄HCO₃), B: ACN; Gradient: 5% B increasing to 95% B over 1.2 min. Flow Rate: 2.0 mL/min;

LCMS CP Method C1: Column: XBridge SB-C18 3.0×50 mm, 3.5 μm; Mobile Phase: A: Water (10 mM NH₄HCO₃), B: ACN; Gradient: 5% B increasing to 95% B over 1.4 min. Flow Rate: 2.0 mL/min;

LCMS CP Method C2: Column: XBridge SB-C18 4.6*50 mm, 3.5 μm; Mobile Phase: A: Water (10 mM NH₄HCO₃), B: ACN; Gradient: 5% B increase to 95% B over 1.4 min. 95% B for 1.6 min. Flow Rate: 2.0 mL/min;

LCMS CP Method D: Column: XBridge SB-C18 3.0×50 mm, 3.5 μm; Mobile Phase: A: Water (0.1% v/v TFA), B: ACN (0.1% v/v TFA); Gradient: 5% B increasing to 95% B over 3.1 min. Flow Rate: 1.8 mL/min;

LCMS CP Method E: Column: XBridge SB-C18 3.0×50 mm, 3.5 μm; Mobile Phase: A: Water (0.1% v/v TFA), B: ACN (0.1% v/v TFA); Gradient: 5% B increasing to 95% B over 1.8 min, stopping at 3 min. Flow Rate: 1.8 mL/min;

LCMS CP Method F: Column: XBridge SB-C18 3.0×50 mm, 3.5 μm; Mobile Phase: A: Water (0.1% v/v TFA), B: ACN (0.1% v/v TFA); Gradient: 5% B increasing to 95% B over 2 min, stop at 3 min. Flow Rate: 1.8 mL/min;

LCMS CP Method G: Apparatus: Agilent 1260 Bin. Pump: G1312B, degasser; autosampler, ColCom, DAD: Agilent G1315D, 220-320 nm, MSD: Agilent LC/MSD G6130B ESI, pos/neg 100-1000, ELSD Alltech 3300 gas flow 1.5 ml/min, gas temp: 40° C.; column: Waters XSelect™ C18, 50×2.1 mm, 3.5 μm, Temp: 35° C., Flow Rate: 0.8 mL/min, Gradient: to =5% A, t 3.5 min=98% A, t 6 min=98% A, Post time: 2 min; Mobile Phase A: 0.1% v/v formic acid in acetonitrile, Mobile Phase B: 0.1% v/v formic acid in water).

### General Procedure GP-1 for Urea Synthesis:

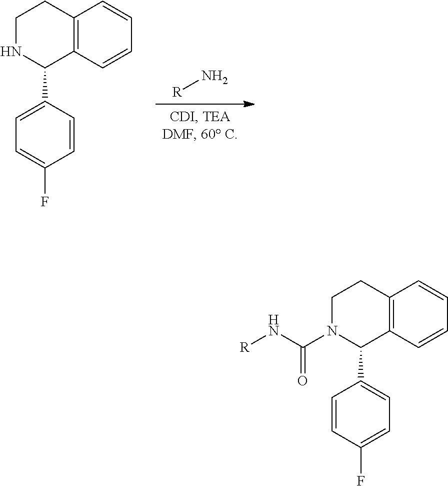

To a solution of (S)-1-(4-fluorophenyl)-1,2,3,4-tetrahydroisoquinoline (340 mg, 1.50 mmol, 1 eq) in DMF (2 mL) was added the amine (2.3 mmol 1.5 eq), CDI (158 mg, 3.0 mmol, 2 eq) and TEA (303 mg, 3.0 mmol, 2 eq). The mixture was stirred at 60° C. for 60 min then cooled to 25° C. and added water (20 mL). The mixture was extracted with three 20 mL portions of ethyl acetate. The combined organic layers were washed with brine, dried and concentrated in vacuo to give a crude product. The crude product was purified by prep-HPLC to give the desired urea as a solid.

### General Procedure GP-2 for Carbamate Synthesis:

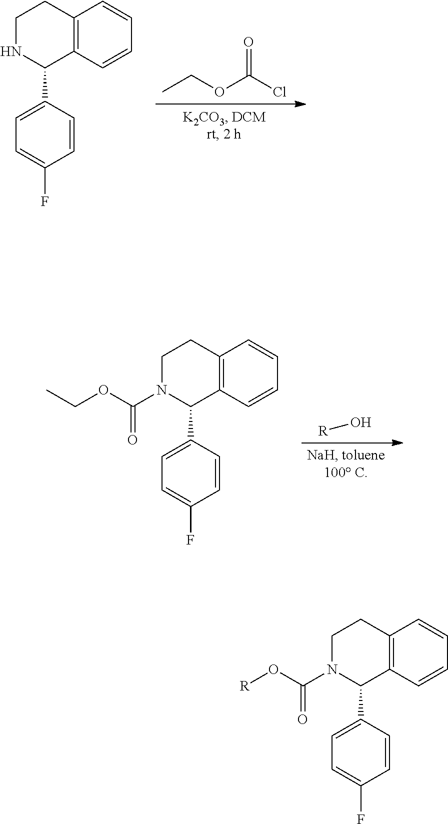

Step 1: To a solution of (S)-1-(4-fluorophenyl)-1,2,3,4-tetrahydroisoquinoline (227 mg, 1 mmol) in DCM (5 mL) was added ethyl carbonochloridate (218 mg, 2 mmol, 2 eq), and potassium carbonate (414 mg, 3 mmol, 3 eq). The mixture was stirred at room temperature for 2 h. The mixture was diluted with 40 mL of water and extracted by three 50 mL portions of DCM. The combined organic layers were dried over Na₂SO₄ and concentrated to give (S)-ethyl 1-(4-fluorophenyl)-3,4-dihydroisoquinoline-2(1H)-carboxylate (299 mg) as a yellow oil which was used for the next step without any further purification.

Step 2: To a mixture of (S)-ethyl 1-(4-fluorophenyl)-3,4-dihydroisoquinoline-2(1H)-carboxylate (299 mg, 1 mmol) in toluene (10 mL) was added the alcohol (1.5 mmol, 1.5 eq) and NaH (80 mg, 2 mmol, 2 eq). The mixture was stirred at 100° C. for 2 h under N₂. The mixture was diluted with water (20 mL) and extracted with three 20 mL portions of dichloromethane. The combined organic layers were washed with brine, dried and concentrated in vacuo to give a crude product. The crude product was purified by prep-HPLC to give the carbamate.

### General Procedure GP-3 for Carbamate Synthesis:

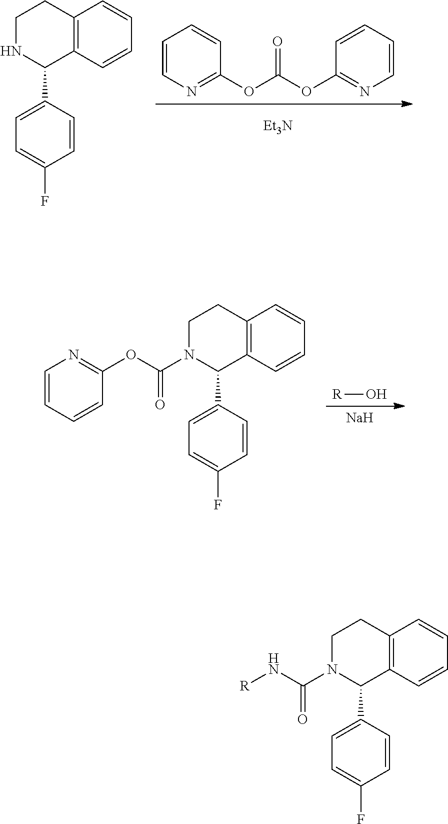

Step 1: To a solution of (S)-1-(4-fluorophenyl)-1,2,3,4-tetrahydroisoquinoline (227 mg, 1 mmol) in DCM (5 mL) was added dipyridin-2-yl carbonate (324 mg, 1.5 mmol, 1.5 eq) and TEA (303 mg, 3 mmol, 3 eq). The mixture was stirred at room temperature for 3 hrs. The mixture was cooled to 25° C. and water was added (40 mL). Then the mixture was extracted with three 20 mL portions of DCM. The combined organic layers were washed with brine, dried and concentrated in vacuo to give a crude product. The crude product was used in the next step without any further purification.

Step 2: To a solution of (S)-pyridin-2-yl 1-(4-fluorophenyl)-3,4-dihydroisoquinoline-2(1H)-carboxylate (348 mg, 1 mmol) in toluene (5 mL) was added the alcohol (1.2 mmol, 1.2 eq) and NaH (48 mg, 1.2 mmol). The mixture was stirred at 60° C. The mixture was cooled to 25° C. and water was added (20 mL). The mixture was extracted with three 20 mL portions of ethyl acetate. The combined organic layers were washed with brine, dried and concentrated in vacuo to give a crude product. The crude product was purified by prep-HPLC to give the carbamate.

### General Procedure GP-4 for Carbamate Synthesis:

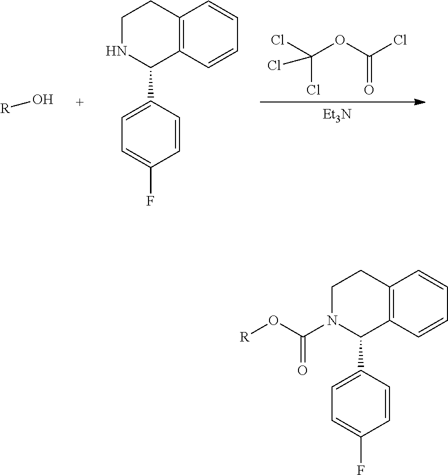

To a solution of the alcohol (1 mmol, 1 eq) in MeCN (5 mL) was added trichloromethyl carbonochloridate (198 mg, 1 mmol, 1 eq) and the mixture was stirred at room temperature for 2 h. The mixture was concentrated to give a white solid. The white solid was dissolved in 5 mL of DMF and (S)-1-(4-fluorophenyl)-1,2,3,4-tetrahydroisoquinoline (227 mg, 1 mmol) and TEA (303 mg, 3 mmol, 3 eq) was added. The mixture was stirred at 90° C. overnight. The mixture was cooled to 25° C. and water (10 mL) was added. The mixture was extracted with three 20 mL portions of ethyl acetate. The combined organic layers were washed with brine, dried and concentrated in vacuo to give a crude product. The crude product was purified by prep-HPLC to give the carbamate.

### General Procedure GP-5 for Amide Synthesis:

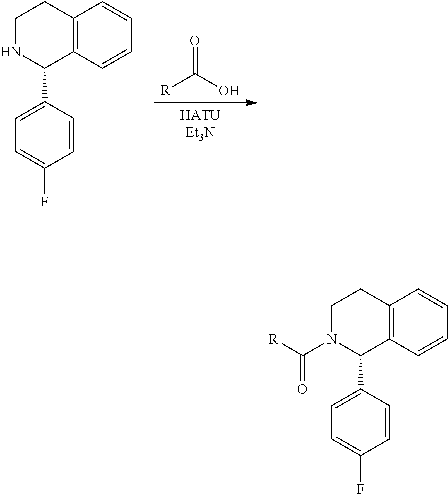

To a solution of the acid (1 mmol) in DMF (5 mL) was added (S)-1-(4-fluorophenyl)-1,2,3,4-tetrahydroisoquinoline (250 mg, 1.1 mmol), HATU (418 mg, 1.1 mmol) and TEA (202 mg, 2 mmol). The mixture was stirred at 25° C. for 1 hour. To the mixture was added water (20 mL) and extracted with three 10 mL portions of ethyl acetate. The combined organic layers were washed with brine, dried and concentrated in vacuo to give a crude product. The crude product was purified by prep-HPLC to give the amide.

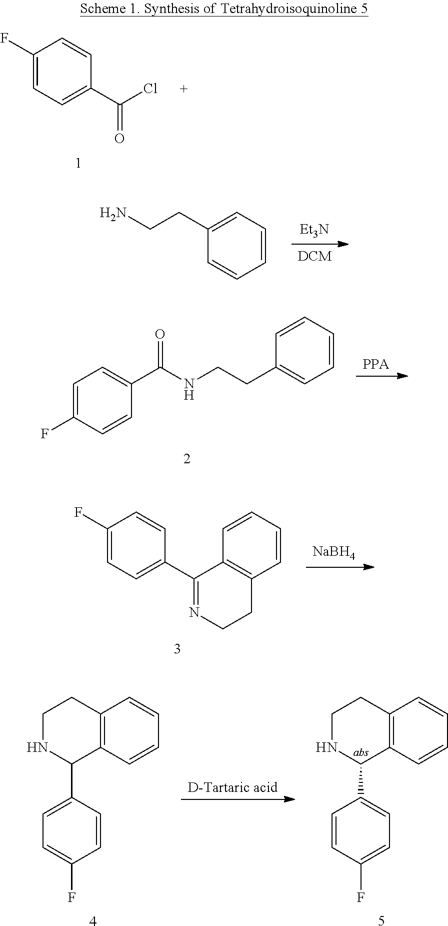

Step 1: To a solution of 2-phenylethanamine (400 g, 3.3 mol) in dichloromethane (4 L) was added 4-fluorobenzoyl chloride (522 g, 3.3 mol) at 0° C. Triethylamine (434 g, 4.3 mol) was added to the white reaction suspension at 0° C. The mixture was stirred for 2 hours at room temperature. Water (4 L) was added and the phases were separated. The organic phase was washed with brine (2 L) and dried over Na₂SO₄. The solvent was evaporated to give 760 g of 4-fluoro-N-phenethylbenzamide 2 as a yellow solid.

Step 2: A round bottomed flask was charged with 500 mL of PPA. The material was heated to 160° C., then 4-fluoro-N-phenethylbenzamide 2 (350 g, 1.44 mol) was added. The mixture was stirred at 160° C. for 3 hours. The mixture was cooled to 25° C. and 3 L of water was added. The mixture was alkalized with NaOH (20% aq.) to pH 11 and extracted with three 1 L portions of ethyl acetate. The combine organic layers were washed three times with brine, dried and concentrated in vacuo to give crude product. The crude product was purified by column chromatography eluting with petroleum ether/ethyl acetate (3:1) to give 273 g of 1-(4-fluorophenyl)-3,4-dihydroisoquinoline 3 as a yellow solid.

Step 3: To a solution of 1-(4-fluorophenyl)-3,4-dihydroisoquinoline 3 (273 g, 1.2 mol) in MeOH (3000 mL) was added NaBH₄ (138 g, 3.6 mol) at room temperature. The mixture was stirred at room temperature for 0.5 h. The mixture was concentrated in vacuo and the solid was dissolved in ethyl acetate (1000 mL). The mixture was washed with water (1000 mL) and brine (500 mL), dried over Na₂SO₄ and concentrated to give 232 g of 1-(4-fluorophenyl)-1,2,3,4-tetrahydroisoquinoline 4 as a yellow solid.

Note: Additional related racemic 1-aryl tetrahydroisoquinolines were prepared analogously.

Step 4: To a solution of racemic 1-(4-fluorophenyl)-1,2,3,4-tetrahydroisoquinoline 4 (232 g, 1022 mmol) in isopropanol (2 L) was added dropwise a solution of D-Tartaric acid (200 g, 1329 mmol) in isopropanol (1 L) at room temperature. The mixture was stirred at room temperature overnight. The precipitate was filtered and the cake was washed with isopropanol (200 mL) to give a white solid (370 g). The solid was added into isopropanol (2 L) and heated to 100° C. Water was added dropwise (0.6 L) at 100° C. until the solid was dissolved. The mixture was allowed to crystallize at room temperature overnight. The precipitate was isolated by filtration and the cake was washed with isopropanol (200 mL) to give white solid (190 g). A second recrystallization from isopropanol and water (˜3/1, 100° C. to room temperature overnight) afforded 150 g of a white solid. The solid was dissolved in water (500 mL), alkalized with NaOH (20% aq.) to pH 11 and extracted with three 200 mL portions of ethyl acetate. The combine organic layers were washed with brine (0.5 L), dried and concentrated in vacuo to give (S)-1-(4-fluorophenyl)-1,2,3,4-tetrahydroisoquinoline 5.

Chiral SFC: CO₂/MeOH containing 0.2% ammonia over CHIRALPAK® IG column (4.6*100 mm 5 μm), retention time=6.08 min), 100% ee.

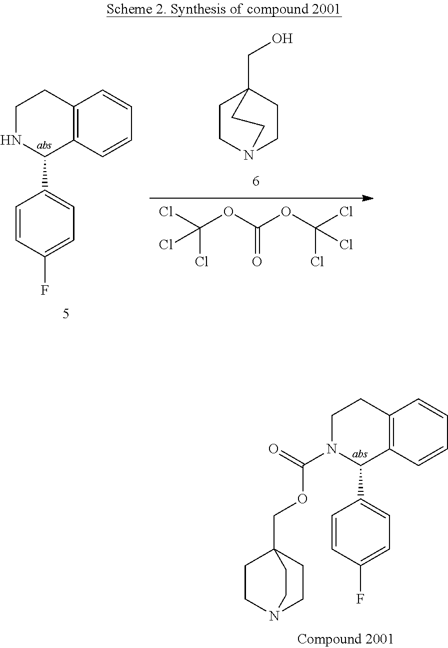 [SMILES: Fc1ccc([C@@H]2NCCc3ccccc32)cc1 ; O=C(OC(Cl)(Cl)Cl)OC(Cl)(Cl)Cl ; OCC12CCN(CC1)CC2 ; O=C(OCC12CCN(CC1)CC2)N1CCc2ccccc2[C@@H]1c1ccc(F)cc1]

To a solution of quinuclidin-4-ylmethanol 6 (282 mg, 2 mmol) in MeCN (10 mL) was added trichloromethyl carbonochloridate (293 mg, 1 mmol) and the mixture was stirred at room temperature for 2 h. The mixture was concentrated to give a white solid. The white solid was dissolved in 5 mL of DMF and (S)-1-(4-fluorophenyl)-1,2,3,4-tetrahydroisoquinoline 5 (114 mg, 0.5 mmol) and TEA (200 mg, 2 mmol) was added. The mixture was stirred at 40° C. for 2 h. The mixture was cooled to 0° C. and water (100 mL) was added. The mixture was extracted with two 50 mL portions of ethyl acetate. The combined organic layers were washed with 50 mL of brine, dried and concentrated in vacuo to give crude product. The crude product was purified by column chromatography eluting with petroleum ether:ethyl acetate (3:1) to give 60 mg of (S)—((S)-quinuclidin-3-yl) 1-(4-fluorophenyl)-3,4-dihydroisoquinoline-2(1H)-carboxylate, compound 2001 as a white solid.

Compound 2001: LCMS: (M+H)⁺=395; purity=100% (214 nm); retention time=1.434 min. CP Method C

¹H NMR (400 MHz, DMSO-d₆) δ 7.23-7.12 (m, 8H), 6.24 (s, 1H), 3.84 (dt, J=12.9, 5.4 Hz, 1H), 3.76 (d, J=10.5 Hz, 1H), 3.70-3.44 (m, 2H), 2.94-2.78 (m, 2H), 2.71-2.68 (brs, 6H), 1.30-1.26 (brs, 6H).

Chiral SFC: CO₂/MeOH containing 0.2% ammonia over CHIRALPAK® IG column (4.6*100 mm 5 μm), retention time=3.40 min), 98.5%.

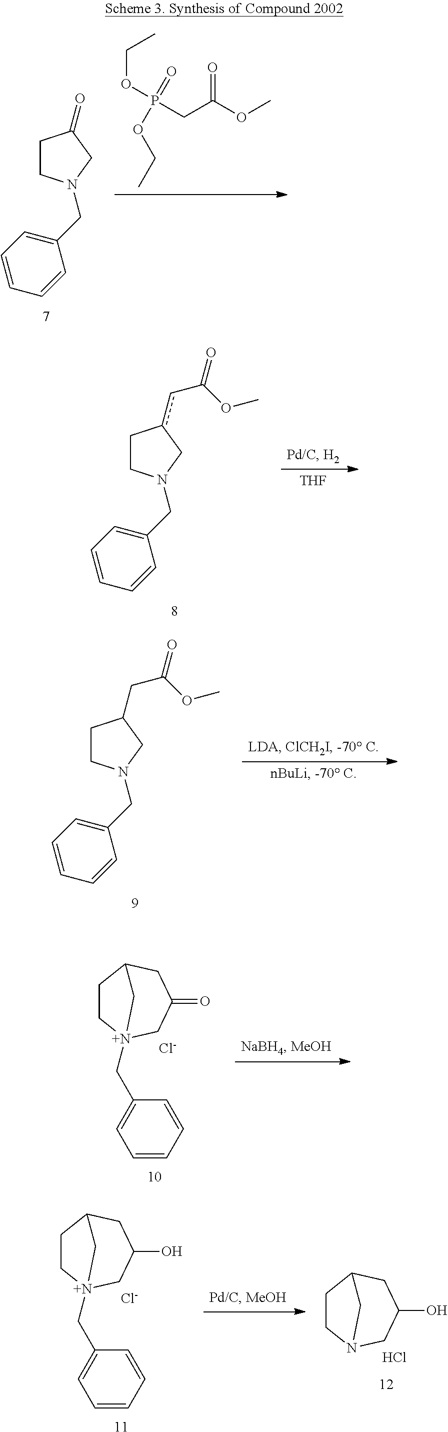

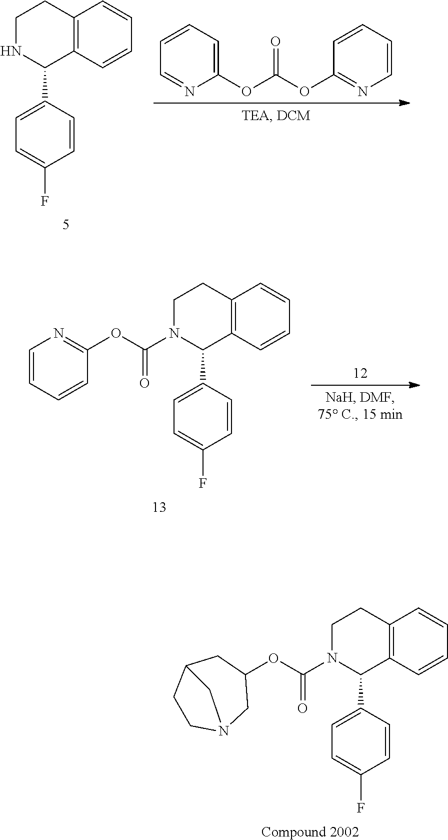

Step 1: To a solution of methyl 2-(diethoxyphosphoryl)acetate (6.6 g, 31.4 mmol) in dry THF (50 mL) was added NaH (1.26 g, 31.4 mmol) at 0° C. After stirring for 20 min, a solution of 1-benzylpyrrolidin-3-one 7 (5 g, 28.5 mmol) in dry THF (5 mL) was added dropwise to the reaction at 0° C. and the reaction was allowed to warm to room temperature and stirred for 16 h. The reaction was poured into ice-water (60 mL) and extracted with three 50 mL portions of EtOAc. The combined organic layers were dried over Na₂SO₄, filtered and concentrated. The residue was purified by silica gel column chromatography eluting with a gradient of petroleum ether:EtOAc from 20:1 to 10:1 to give unsaturated E and Z methyl 2-(1-benzylpyrrolidin-3-ylidene)acetate 8 (6 g, 25.9 mmol) as light yellow oil.

LCMS: (M+1)⁺=232; Retention time=1.775 min. CP Method C

Step 2: To the unsaturated esters 8 (6 g, 25.9 mmol) dissolved in MeOH (50 mL). was added Pt/C (0.51 g, 2.6 mmol). The reaction mixture was evacuated and then refilled with hydrogen. The mixture was stirred for 2 days. The reaction was filtered and concentrated to give crude methyl 2-(1-benzylpyrrolidin-3-yl)acetate 9 (4.77 g, 20.4 mmol) as colorless oil used for next step without further purification.

LCMS: (M+1)⁺=234; Retention time=1.462 min. CP Method C2

Step 3: To a solution of methyl 2-(1-benzylpyrrolidin-3-yl)acetate 9 (4.77 g, 20.4 mmol) in dry THF (50 mL) was added dropwise ClCH₂I (10.82 g, 61.3 mmol) at −70° C. The reaction was stirred for 5 min, then LDA (31 mL, 61.3 mmol, 2 mol/L in THF) was added slowly. After addition (20 min), the mixture was stirred at −70° C. for 25 min. n-BuLi (7.3 mL, 18.2 mmol, 2.5 mol/L in hexane) was added over a 10 min period. The reaction was stirred additional 15 min at −70° C., then saturated NH₄Cl solution (15 mL) was added slowly and the reaction was warmed to 0° C. The mixture was separated, and the organic layer was concentrated to give crude compound. The crude compound was dissolved in EtOAc (30 mL) and the solution was washed with two 50 mL portions of water. The aqueous layers were concentrated to give crude 1-benzyl-3-oxo-1-azoniabicyclo[3.2.1]octane chloride 10 (1.63 g, 6.5 mmol) as yellow solid used for next step without further purification.

LCMS: (M+1)⁺=216; Retention time=0.83 min. CP Method C2.

Step 4: To a solution of 1-benzyl-3-oxo-1-azoniabicyclo[3.2.1]octane chloride 10 (1.43 g, 5.68 mmol) in MeOH (20 mL) was added slowly NaBH₄ (0.43 g, 11.4 mmol) at 0° C. The reaction was stirred at rt for 2 h. The reaction was purified by prep-HPLC to give 1-benzyl-3-hydroxy-1-azoniabicyclo[3.2.1]octane 11 (0.91 g, 3.6 mmg) as white solid.

LCMS: (M+1)⁺=218; Retention time=0.88 min, 0.938 min. CP Method C2.

Step 5: To a solution of 1-benzyl-3-hydroxy-1-azoniabicyclo[3.2.1]octane 11 (0.91 g, 3.6 mmg) (0.91 g, 3.58 mmol) in MeOH (20 mL) was added 10% Pd/C (200 mg, 0.2 mmol) at rt. The reaction mixture was evacuated and then refilled with hydrogen. The reaction mixture was stirred for 16 h. The reaction mixture was filtered and the filtrate was concentrated to give 1-azabicyclo[3.2.1]octan-3-ol hydrochloride 12 (386 mg, 2.36 mmol) as colorless solid.

LCMS: (M+1)⁺=127; Retention time=0.37 min. CP Method C.

Step 6: To a solution of (S)-1-(4-fluorophenyl)-1,2,3,4-tetrahydroisoquinoline 5 (215 mg, 0.26 mmol) and TEA (0.26 mL, 1.88 mmol) in DCM (30 mL) was added dipyridin-2-yl carbonate (407 mg, 1.88 mmol). The reaction mixture was stirred at rt for 16 h. The reaction was diluted with DCM (60 mL) and then washed with water, brine, dried over Na₂SO₄, and concentrated to give (S)-pyridin-2-yl 1-(4-fluorophenyl)-3,4-dihydroisoquinoline-2(1H)-carboxylate 13 (324 mg, 0.0.93 mmol) as light yellow oil for next step without further purification.

LCMS: (M+1)⁺=349; Retention time=1.902 min. CP Method A2.

Step 7: To a solution of 1-azabicyclo[3.2.1]octan-3-ol hydrochloride 12 (100 mg, 0.6 mmol) in dry DMF (3 mL) was added NaH (96 mg, 2.4 mmol, 60% in mineral oil) at 0° C. After stirring for 20 min at rt, the reaction mixture was cooled to 0° C. and a solution of (S)-pyridin-2-yl 1-(4-fluorophenyl)-3,4-dihydroisoquinoline-2(1H)-carboxylate 13 (277 mg, 0.79 mmol) in dry DMF (1.5 mL) was added. The reaction was heated to 75° C. for 15 min. The reaction mixture was cooled and poured into ice water. The mixture was extracted with three 30 mL portions of EtOAc. The combined organic layers were dried over Na₂SO₄, filtered and concentrated. The crude compound was purified by prep-HPLC to give compound 2002.

Compound 2002: LCMS: (M+1)⁺=381; Retention time=1.463 min. CP Method A2.

¹HNMR (400 Hz, CD₃OD): δ 7.24-7.17 (m, 5H), 7.02 (t, J=7.4 Hz, 3H), 6.32-6.20 (m, 1H), 5.19-5.11 (m, 1H), 4.00-3.96 (m, 1H), 3.56-3.49 (m, 1H), 3.44-3.32 (m, 2H), 3.28-3.22 (m, 2H), 3.07-2.69 (m, 5H), 2.24-2.10 (m, 2H), 2.01-1.95 (m, 1H), 1.82-1.74 (m, 1H).

### Chiral Analysis:

CO₂/MeOH containing 0.2% ammonia over OJ-H column (4.6*100 mm 5 μm), retention time=3.11 min, 98.6%.

CO₂/MeOH containing 0.2% ammonia over AD-H column (4.6*100 mm 5 μm); retention time=1.26 min, 97.3%.

CO₂/MeOH containing 0.2% ammonia over EnantioPak AS column (4.6*100 mm 5 μm), retention time=1.83 min, 94.7%.

The following compound was prepared using General Procedure GP-5:

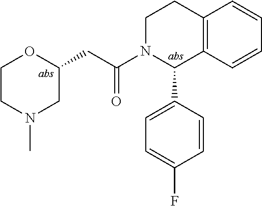 [SMILES: CN1CCO[C@H](CC(=O)N2CCc3ccccc3[C@@H]2c2ccc(F)cc2)C1]

Compound 2003: LCMS: (M+H)⁺=369; purity=98% (214 nm); retention time=1.420 min. CP Method A1

¹H NMR (400 MHz, CD₃OD) δ 7.30-7.13 (m, 5H), 7.09-6.95 (m, 3H), 6.84 (s, 1H), 4.06-3.89 (m, 2H), 3.84-3.73 (m, 1H), 3.62 (dt, J=11.6, 2.4 Hz, 1H), 3.47 (dt, J=10.8, 4.4 Hz, 1H), 3.04 (ddd, J=16.4, 10.8, 5.6 Hz, 1H), 2.88-2.75 (m, 2H), 2.74-2.63 (m, 2H), 2.56 (dd, J=15.2, 4.8 Hz, 1H), 2.28 (s, 3H), 2.10 (dt, J=12.0, 3.6 Hz, 1H), 1.93 (t, J=10.8 Hz, 1H).

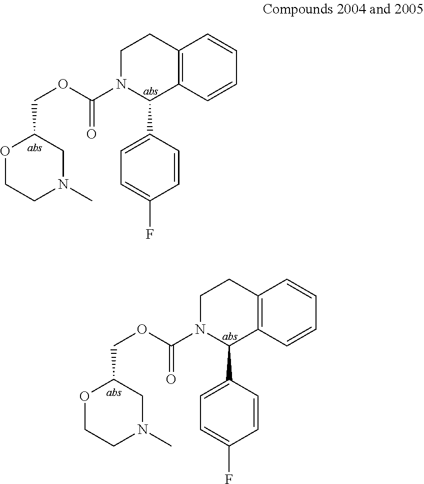 [SMILES: CN1CCO[C@H](COC(=O)N2CCc3ccccc3[C@@H]2c2ccc(F)cc2)C1 ; CN1CCO[C@H](COC(=O)N2CCc3ccccc3[C@H]2c2ccc(F)cc2)C1]

The diastereomers were separated by chiral SFC eluting with CO₂/EtOH containing 0.5 methanol ammonia over an EnantioPak® AD column (20*250 mm 10 μm) to give compound 2004 (19.5 mg, retention time=2.05 min) and compound 2005 (49.5 mg, retention time=1.64 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2004: LCMS: (M+H)⁺=385; retention time=1.449 min. CP Method A1

¹H NMR (400 MHz, CD₃OD) δ 7.30-7.17 (m, 5H), 7.09-6.99 (m, 3H), 6.33 (s, 1H), 4.32-4.09 (m, 2H), 4.07-3.98 (m, 1H), 3.91 (d, J=11.2 Hz, 1H), 3.82-3.75 (m, 1H), 3.66 (dt, J=11.6, 2.0 Hz, 1H), 3.39-3.29 (m, 1H), 2.97 (ddd, J=16.0, 8.4, 6.0 Hz, 1H), 2.89-2.76 (m, 2H), 2.72 (d, J=11.6 Hz, 1H), 2.31 (s, 3H), 2.17 (dt, J=11.6, 3.6 Hz, 1H), 1.96-1.93 (m, 1H).

Compound 2005: LCMS: (M+H)⁺=385; retention time=1.450 min. CP Method A1

¹H NMR (400 MHz, CD₃OD) δ 7.30-7.17 (m, 5H), 7.08-6.98 (m, 3H), 6.33 (s, 1H), 4.34-4.07 (m, 2H), 4.06-3.96 (m, 1H), 3.92-3.89 (m, 1H), 3.82-3.73 (m, 1H), 3.65 (t, J=11.4, 1H), 3.37-3.27 (m, 1H), 2.95 (ddd, J=16.4, 10.0, 5.6 Hz, 1H), 2.89-2.73 (m, 2H), 2.70 (d, J=12.0 Hz, 1H), 2.28 (s, 3H), 2.13 (dt, J=11.6, 3.2 Hz, 1H), 1.92 (t, J=10.8 Hz, 1H).

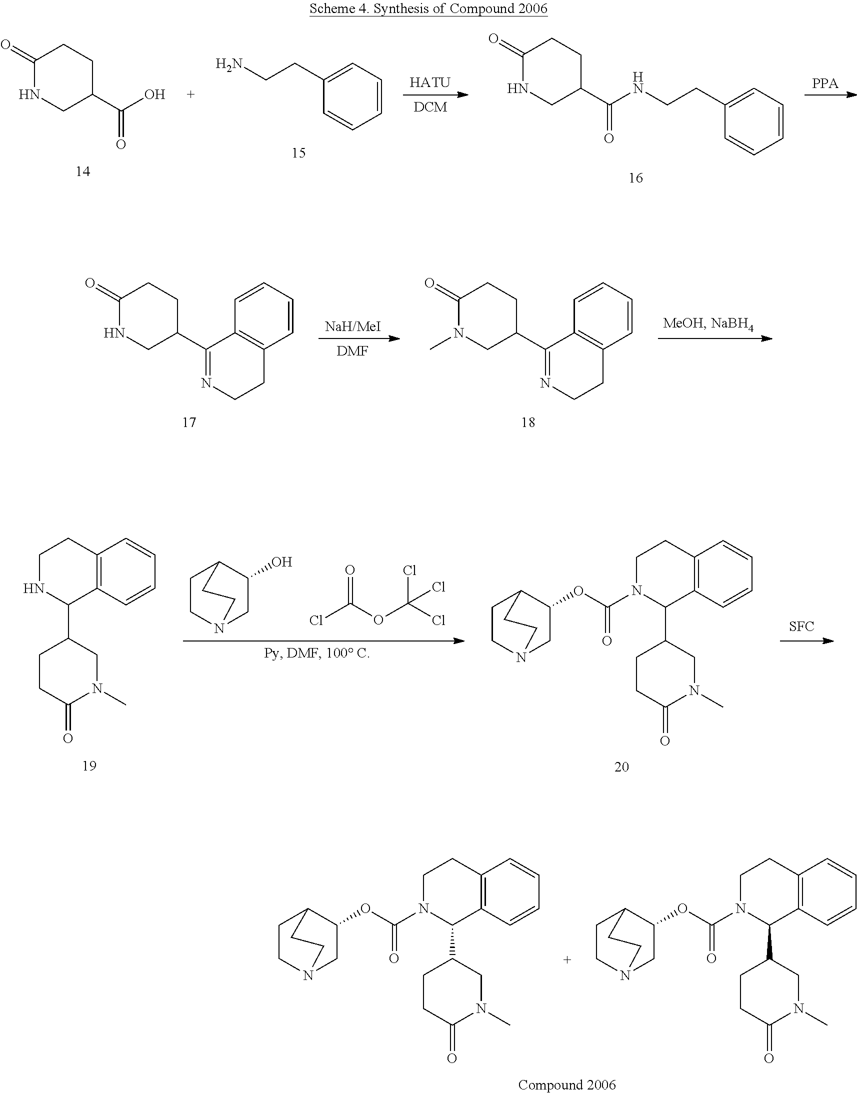

Steps 1: To a solution of 6-oxopiperidine-3-carboxylic acid 14 (1430 mg, 10 mmol, 1 equiv) in DCM (50 mL) was added 2-phenylethanamine (1210 mg, 10 mmol, 1.0 equiv), HATU (5700 mg, 15 mmol, 1.5 equiv) and TEA (3030 mg, 30 mmol, 3.0 equiv). After stirring at 25° C. for 1 hour, water (50 mL) was added. It was extracted with three 100 mL portions of ethyl acetate. The combined organic layers were washed with brine, dried (Na₂SO₄) and concentrated in vacuo to give a crude product 16 (2.2 g). It was used for the next step without any further purification.

LCMS: (M+H)⁺=247 (UV 214 nm); Retention time=1.2 min. CP Method C

Step 2: PPA (3 ml) was added to a round-bottom flask and it was heated to 140° C. 6-oxo-N-phenethylpiperidine-3-carboxamide 16 (2 g, 8.1 mmol) was added. The mixture was stirred at 140° C. for 5 hours. The reaction mixture was cooled to 80° C. and poured into ice water, the pH was adjusted to 11 by 1N NaOH aq., and the mixture was extracted with three 80 mL portions of ethyl acetate. The combined organic phases were washed by brine (150 ml), dried over anhydrous Na₂SO₄. The solution was concentrated to obtain 5-(3, 4-dihydroisoquinolin-1-yl)piperidin-2-one 17 (1.1 g).

LCMS: (M+H)⁺=229 (UV 214 nm); Retention time=0.88 min. CP Method C

Step 3: To a mixture of 5-(3, 4-dihydroisoquinolin-1-yl) piperidin-2-one 17 (1.14 g, 5 mmol) in DMF (30 ml) was added NaH (60% in mineral oil) (240 mg, 6 mmol) at 0° C. and the resulting mixture was stirred for 30 min at 25° C. Mel (710 mg, 5 mmol) was added slowly and the reaction mixture was stirred at 25° C. for 3 hr. The reaction mixture was partitioned between ethyl acetate (100 mL) and brine (200 mL). The water layer was extracted with two 100 mL portions of ethyl acetate. The combined organic layers were washed with brine, dried over sodium sulfate, filtered and concentrated. The residue was purified by a silica gel column using an EtOAc/PE gradient to afford 5-(3,4-dihydroisoquinolin-1-yl)-1-methylpiperidin-2-one 18 (440 mg).

LCMS: (M+H)⁺=243 (UV 214 nm); Retention time=0.924 min. CP Method C

Step 4: To a solution of 5-(3,4-dihydroisoquinolin-1-yl)-1-methylpiperidin-2-one 18 (440 mg, 1.8 mmol) in 5 mL of MeOH, NaBH₄(183 mg, 5.4 mmol) was slowly added at 0° C. The mixture was stirred for one hour. The reaction was quenched with 10 mL of water. The mixture was extracted with three 10 mL portions of DCM. The combined organic layers were washed with 10 mL of brine, dried over sodium sulfate, filtered and concentrated to afford 1-methyl-5-(1,2,3,4-tetrahydroisoquinolin-1-yl)piperidin-2-one 19 (430 mg).

LCMS: (M+H)⁺=245 (UV 214 nm); Retention time=0.932 min. CP Method C

Step 5: (S)-quinuclidin-3-yl 1-(1-methyl-6-oxopiperidin-3-yl)-3,4-dihydroisoquinoline-2(1H)-carboxylate 20 was prepared following the General Procedure GP-4 for carbamate synthesis.

The diastereomers were separated by chiral SFC eluting with MeOH containing 0.2% Methanol Ammonia over an EnantioPak® IG column (4.6*100 mm 5 μm) to give compound 2006 as a mixture of isomers (retention time=2.45-2.77 min) Stereochemical assignment of (S) at quinuclidine is absolute based on starting materials.

Compound 2006: LCMS: (M+H)⁺=459; purity=100% (214 nm); Retention time=1.344 min. CP Method A

¹H NMR (400 MHz, DMSO-d₆) δ 7.17 (dd, J=37.0, 29.2 Hz, 4H), 4.87 (dd, J=23.9, 9.9 Hz, 1H), 4.60 (s, 1H), 3.88-3.69 (m, 1H), 3.67-3.43 (m, 1H), 3.29-3.17 (m, 2H), 3.15-2.85 (m, 3H), 2.83-2.68 (m, 5H), 2.60 (t, J=14.7 Hz, 2H), 2.35-2.17 (m, 2H), 2.16-2.05 (m, 1H), 2.04-1.94 (m, 1H), 1.89-1.72 (m, 1H), 1.63-1.53 (br, s, 2H), 1.51-1.43 (br, s, 1H), 1.34 (d, J=10.9 Hz, 1H), 1.27-1.18 (br, s, 1H).

Chiral SFC: MeOH (containing 0.2% Methanol Ammonia) over an ENANTIOPAK® IG column (4.6*100 mm 5 μm), retention time=2.45-2.77 min) diastereomeric ratio ˜3:1.

The following compounds were prepared following General Procedure GP-4 for carbamate synthesis

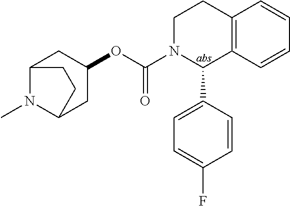

Compound 2007: isolated as a mixture of isomers: LCMS: (M+H)⁺=395; purity=100% (214 nm); retention time=1.928 min. CP Method C

¹H NMR (400 MHz, DMSO-d₆) δ 7.28-7.03 (m, 8H), 6.20 (s, 1H), 4.87-4.76 (m, 1H), 4.00-3.75 (m, 1H), 3.27-3.19 (m, 1H), 3.10 (s, 2H), 2.92-2.72 (m, 2H), 2.19 (s, 3H), 1.96-1.85 (m, 2H), 1.80-1.70 (m, 2H), 1.69-1.61 (m, 1H), 1.60-1.46 (m, 3H).

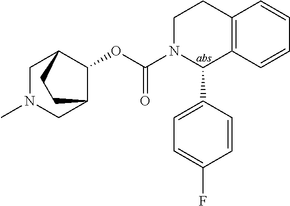 [SMILES: CN1C[C@H]2CC[C@@H](C1)[C@H]2OC(=O)N1CCc2ccccc2[C@@H]1c1ccc(F)cc1]

Compound 2008: LCMS: (M+H)⁺=395; purity=100% (214 nm); retention time=1.460 min. CP Method C

¹H NMR (400 MHz, CD₃OD) δ 7.36-7.19 (m, 5H), 7.12-7.01 (m, 3H), 6.38 (s, 1H), 4.74 (s, 1H), 4.15-3.85 (br, 1H), 3.55-3.37 (br, 1H), 3.07-2.93 (br, 1H), 2.91-2.86 (tt, J=4.4 Hz, 4.8 Hz, 1H), 2.61-2.36 (m, 4H), 2.31-2.10 (m, 5H), 1.79 (s, 4H).

The following compound was prepared using General Procedure GP-5 for amide synthesis:

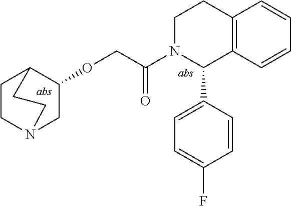 [SMILES: O=C(CO[C@@H]1CN2CCC1CC2)N1CCc2ccccc2[C@@H]1c1ccc(F)cc1]

Compound 2009: LCMS: (M+H)⁺=395; purity=96.59% (214 nm); retention time=1.428 min. CP Method A2

¹H NMR (400 MHz, CD₃OD) δ 7.27-7.22 (m, 5H), 7.10-7.01 (m, 3H), 6.77 (s, 1H), 4.41 (t, J=14.8 Hz, 2H), 4.00-3.95 (m, 1H), 3.79-3.75 (m, 1H), 3.57-3.46 (m, 2H), 3.34-3.26 (m, 5H), 3.09-3.04 (m, 1H), 2.91-2.87 (m, 1H), 2.44-2.39 (m, 1H), 2.21-2.17 (m, 1H), 2.04-2.00 (m, 1H), 1.83-1.78 (m, 2H).

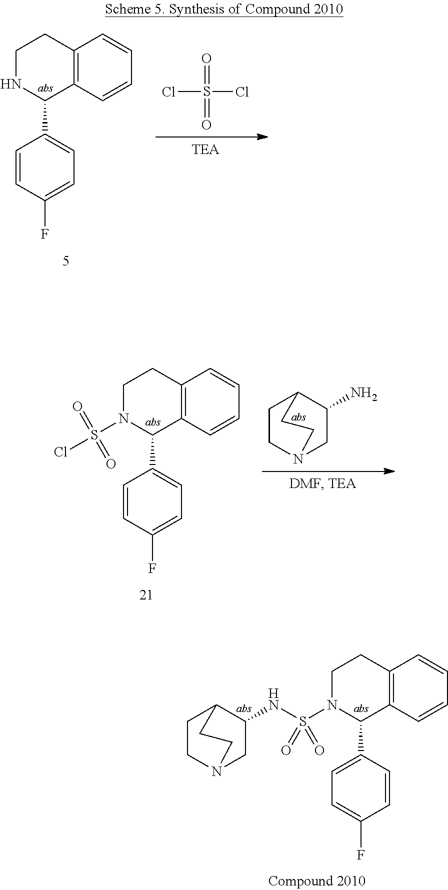

Step 1: To a solution of (S)-1-(4-fluorophenyl)-1,2,3,4-tetrahydroisoquinoline 5 (227 mg, 10 mmol) in dichloromethane (20 mL) was added the sulfuryl dichloride (200 mg, 15 mmol) at 0° C. Triethylamine (300 mg, 30 mmol) was added to the reaction suspension at 0° C. The mixture was stirred for 2 hours at room temperature. The mixture was evaporated to (S)-1-(4-fluorophenyl)-3,4-dihydroisoquinoline-2(1H)-sulfonyl chloride 21 as a yellow solid.

Step 2: To a solution of (S)-1-(4-fluorophenyl)-3,4-dihydroisoquinoline-2(1H)-sulfonyl chloride 21 (325 mg, 10 mmol) in DMF (20 mL) was added (S)-quinuclidin-3-amine (127 mg, 10 mmol) at room temperature. Triethylamine (300 mg, 30 mmol) was added to the reaction suspension at 0° C. The mixture was stirred for 2 hours at room temperature. Water (40 mL) was added and the phases were separated. The organic phase was washed with two 100 mL portions of brine and dried over Na₂SO₄. The solvent was removed and the product was purified by HPLC to give compound 2010.

Compound 2010: LCMS: (M+H)⁺=416; purity=100% (214 nm); retention time=1.749 min. CP Method C

¹H NMR (400 MHz, DMSO-d₆) δ 8.36 (s, 1H), 7.33-7.01 (m, 8H), 5.97 (s, 1H), 3.61 (d, J=11.2 Hz, 1H), 3.27 (t, J=9.7 Hz, 3H), 3.02 (dd, J=18.8, 8.1 Hz, 4H), 2.89-2.77 (m, 3H), 1.89-1.80 (m, 1H), 1.74-1.65 (d, J=11.6 Hz, 1H), 1.58-1.48 (m, 1H), 1.44-1.35 (m, 1H), 1.25 (d, J=8.6 Hz, 1H).

The following compounds were prepared analogously to compound 2002 using enantiomerically pure (S)-quinuclidin-3-ol and racemic tetrahydrosioquinoline:

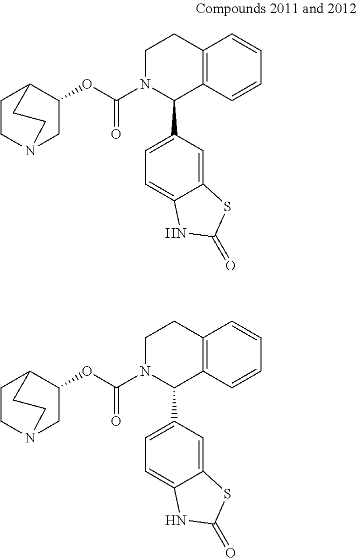 [SMILES: O=C(O[C@@H]1CN2CCC1CC2)N1CCc2ccccc2[C@H]1c1ccc2[nH]c(=O)sc2c1 ; O=C(O[C@@H]1CN2CCC1CC2)N1CCc2ccccc2[C@@H]1c1ccc2[nH]c(=O)sc2c1]

The diastereomers were separated by chiral SFC eluting with CO₂/MeOH containing 0.2% methanolic ammonia over an EnantioPak® AS column (4.6*100 mm 5 μm) to give compound 2011 (retention time=19.03 min) and compound 2012 (retention time=10.74 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2011: LCMS: (M+H)⁺=436; purity=100% (214 nm); retention time=1.379 min. CP Method C

¹H NMR (400 MHz, CD₃OD) δ 7.37-7.31 (br, 1H), 7.28-7.16 (m, 5H), 7.13-7.07 (t, J=8.4 Hz, 2H), 4.87-4.81 (br, 1H), 4.09-4.01 (m, 1H), 3.52-3.36 (br, 1H), 3.30-3.24 (m, 1H), 3.03-2.93 (m, 1H), 2.92-2.83 (br, 4H), 2.82-2.71 (m, 2H), 2.1 (s, 1H), 1.84-1.61 (m, 2H), 1.58-1.44 (br, 1H), 1.38-1.28 (m, 1H).

Chiral SFC: n-Hexane containing 0.1% DEA/EtOH containing 0.1% DEA over CHIRALPAK® IG column (4.6*250 mm 5 μm), retention time=15.183 min), 100% ee.

Compound 2012: LCMS: (M+H)⁺=436; purity=100% (214 nm); retention time=1.377 min. CP Method C

¹H NMR (400 MHz, CD₃OD) δ 7.43-7.31 (m, 1H), 7.28-7.19 (m, 4H), 7.18-7.05 (m, 3H), 6.42-6.22 (m, 1H), 4.87-4.81 (t, J=4.4 Hz, 1H), 4.05-3.85 (m, 1H), 3.63-3.36 (m, 1H), 3.30-3.21 (m, 1H), 3.04-2.93 (m, 1H), 2.94-2.71 (m, 5H), 2.68-2.53 (m, 1H), 2.08 (s, 1H), 1.95-1.84 (m, 1H), 1.83-1.74 (m, 1H), 1.71-1.61 (m, 1H), 1.58-1.47 (m, 1H), 1.38-1.25 (m, 1H).

Chiral SFC: n-Hexane containing 0.1% DEA/EtOH containing 0.1% DEA over CHIRALPAK® IG column (4.6*250 mm 5 μm), retention time=19.916 min), 100% ee.

The following compound was prepared using General Procedure GP-1 coupling conditions and chiral THIQ 5:

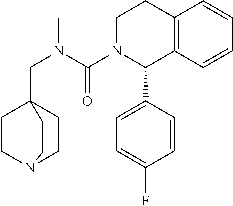 [SMILES: CN(CC12CCN(CC1)CC2)C(=O)N1CCc2ccccc2[C@@H]1c1ccc(F)cc1]

Compound 2013: LCMS: (M+H)⁺=408; purity=100% (214 nm); retention time=1.822 min. CP Method C

¹H NMR (400 MHz, DMSO-d₆) δ 7.24-7.22 (m, 2H), 7.21-7.17 (m, 1H), 7.16-7.09 (m, 5H), 6.54 (t, J=16 Hz, 1H), 6.40 (s, 1H), 3.80-3.70 (m, 1H), 3.30-3.23 (m, 1H), 3.11-3.02 (m, 1H), 2.94-2.88 (m, 2H), 2.87-2.80 (m, 7H), 2.73-2.64 (m, 1H), 1.51-1.37 (m, 7H).

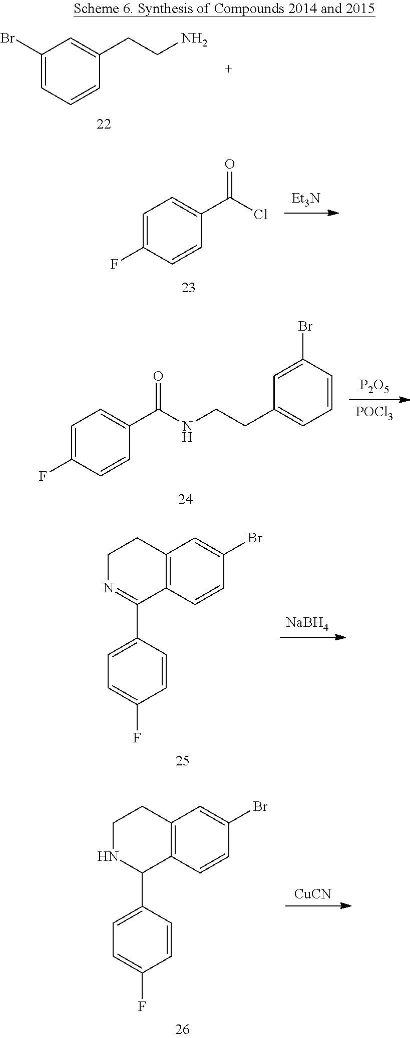

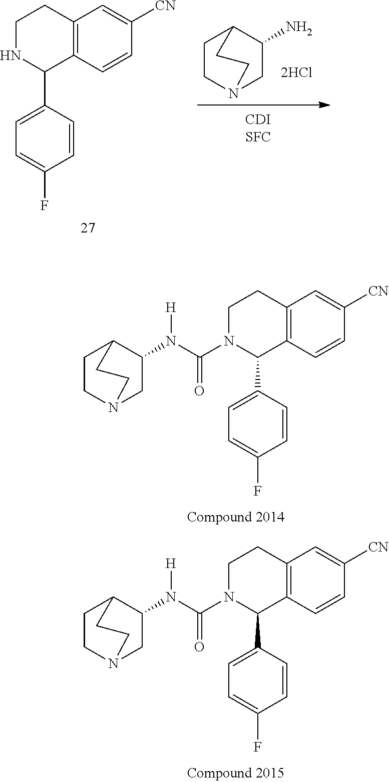 [SMILES: N#Cc1ccc2c(c1)CCNC2c1ccc(F)cc1 ; N[C@@H]1CN2CCC1CC2 ; N#Cc1ccc2c(c1)CCN(C(=O)N[C@@H]1CN3CCC1CC3)[C@H]2c1ccc(F)cc1 ; N#Cc1ccc2c(c1)CCN(C(=O)N[C@@H]1CN3CCC1CC3)[C@@H]2c1ccc(F)cc1]

Step 1: To a solution of 2-(3-bromophenyl)ethylamine 22 (3.05 g, 15.25 mmol) in DCM (50 mL) was added Et₃N (3.2 ml, 22.87 mmol) at 0° C. followed by dropwise addition of 4-fluorobenzoyl chloride (2.78 g, 16.78 mmol) in DCM. The reaction mixture was stirred at 0° C. for 2 h. The reaction was quenched by water and extracted with three 50 mL portions of DCM. The combined organic layers were dried over MgSO₄ and concentrated to give 4.5 g of the expected product N-(3-bromophenethyl)-4-fluorobenzamide 24 as white solid.

LCMS: (M+H)⁺=322 (214 nm); retention time=1.823 min. CP Method A

Step 2: To a mixture of N-(3-bromophenethyl)-4-fluorobenzamide 24 (3.2 g, 9.9 mmol) in 35 mL of POCl₃ was added P2O5 (95 mg, 0.66 mmol) and the reaction was warmed was warmed to 160° C. The mixture was heated under reflux and stirred at 110° C. overnight. The reaction mixture was quenched by the addition of ice water. The pH was adjusted to around 13 by progressively adding solid NaOH. The combined aqueous layers were extracted with three 50 mL portions of DCM. The combined organic layers were dried over MgSO₄ and concentrated to give the expected product 6-bromo-1-(4-fluorophenyl)-3,4-dihydroisoquinoline 25 (2 g) as brown oil.

LCMS: (M+H)⁺=305 (214 nm); retention time=1.373 min. CP Method A

Step 3: 6-bromo-1-(4-fluorophenyl)-3,4-dihydroisoquinoline 25 (2 g, 6.56 mmol) in MeOH (20 mL) was cooled to 0° C., and then NaBH₄ (1 g, 26.24 mmol) was added at rt over 2 h. The mixture was diluted with water (50 mL) and extracted with three 60 mL portions of DCM. The combined DCM layers were dried over Na₂SO₄, and then the solution was concentrated under reduced pressure to give crude 6-bromo-1-(4-fluorophenyl)-1,2,3,4-tetrahydroisoquinoline 26 (2 g).

LCMS: (M+H)⁺=305.7 (254 nm); retention time=1.354 min. CP Method A

Step 4: To a solution of 6-bromo-1-(4-fluorophenyl)-1,2,3,4-tetrahydroisoquinoline 26 (2 g, 6.55 mmol) in DMF (20 mL) warmed to 160° C. was added CuCN (2.3 g, 26.2 mmol). The mixture was heated under reflux and stirred at 160° C. for 6 h. The reaction mixture was quenched by the addition of ice water. The pH was adjusted to around 13 by progressively adding solid NaOH. The combined aqueous layers were extracted with three 50 mL portions of DCM. The combined organic layers were dried over MgSO₄ and concentrated to give 165 mg of 1-(4-fluorophenyl)-1,2,3,4-tetrahydroisoquinoline-6-carbonitrile 27.

LCMS: (M+H)⁺=252.9 (214 nm); retention time=1.257 min. CP Method C

Step 5: To a solution of 1-(4-fluorophenyl)-1,2,3,4-tetrahydroisoquinoline-6-carbonitrile 27 (165 mg, 0.65 mmol) in DMF (5 mL) was added (S)-quinuclidin-3-amine (155 mg, 0.78 mmol), CDI (258 mg, 1.3 mmol), and Et₃N (0.27 ml, 1.95 mmol). The mixture was stirred and heated to 60° C. for 3 h to give the compound (1-R,S)-6-cyano-1-(4-fluorophenyl)-N—((S)-quinuclidin-3-yl)-3,4-dihydroisoquinoline-2(1H)-carboxamide as a white solid.

LCMS: (M+H)⁺=405.1 (254 nm); retention time=1.532 min. CP Method D

The diastereomers were separated by chiral SFC eluting with n-hexane containing 0.1% DEA/EtOH containing 0.1% DEA over an EnantioPak® OD-H column (4.6*250 mm 5 μm) to give compound 2014 (retention time=11.208 min) and compound 2015 (retention time=6.640 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2014: LCMS: (M+H)⁺=405.1; purity=100% (214 nm); retention time=1.603 min. CP Method C

¹H NMR (400 MHz, DMSO-d₆) δ 7.76 (s, 1H), 7.66 (dd, J=7.6 Hz; 1.2, 1H), 7.36 (d, J=8 Hz, 1H), 7.14 (d, J=7.2, 4H), 6.56 (s, 1H), 6.36 (d, J=6 Hz, 1H), 3.92-3.86 (m, 1H), 3.64 (m, 1H), 3.22-3.18 (m, 1H), 2.99-2.91 (m, 2H), 2.78-2.74 (m, 2H), 2.63-2.59 (m, 3H), 2.52-2.48 (m, 1H), 1.74 (s, 2H), 1.52-1.48 (m, 2H), 1.23 (s, 1H).

Chiral SFC: CO₂/MeOH containing 0.2% ammonia over CHIRALPAK® IG column (4.6*250 mm 5 μm), retention time=2.94 min), 100% ee.

Compound 2015: LCMS: (M+H)⁺=405.0; purity=100% (214 nm); retention time=1.621 min. CP Method C

¹H NMR (400 MHz, DMSO-d₆) δ 7.76 (s, 1H) 7.66-7.64 (dd, J=8 Hz; 6.8 Hz, 1H) 7.36 (d, J=8 Hz, 1H), 7.14 (d, J=7.2 Hz, 4H), 6.55 (s, 1H), 6.36 (d, J=6 Hz, 1H), 3.89-3.85 (m, 1H), 3.69-3.65 (m, 1H), 3.35-3.19 (m, 2H), 3.03 (t, J=2.4 Hz, 1H), 2.92-2.88 (m, 1H), 2.81-2.79 (m, 2H), 2.67-2.57 (m, 3H), 1.76-1.72 (m, 2H), 1.53-1.51 (m, 2H), 1.32 (s, 1H).

Chiral SFC: CO₂/MeOH containing 0.2% ammonia over CHIRALPAK® IG column (4.6*250 mm 5 μm), retention time=3.18 min), 100% ee.

The following compounds were made by carbamate coupling using diphosgene, TEA in DMF with chirally pure THIQ 5 and racemic alcohols:

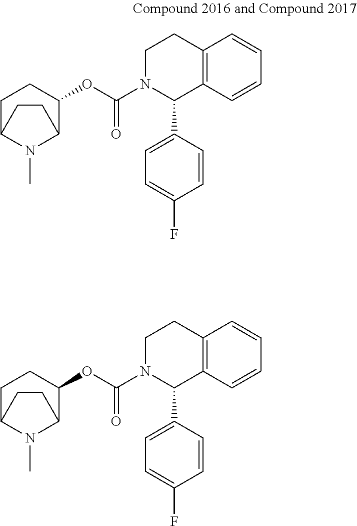 [SMILES: CN1C2CCC1[C@@H](OC(=O)N1CCc3ccccc3[C@@H]1c1ccc(F)cc1)CC2 ; CN1C2CCC1[C@H](OC(=O)N1CCc3ccccc3[C@@H]1c1ccc(F)cc1)CC2]

The diastereomers were separated by chiral SFC eluting with n-hexane containing 0.1% DEA/EtOH containing 0.1% DEA over an EnantioPak® AY column (20*250 mm 10 μm) to give compound 2016 (retention time=7.751 min) and compound 2017 (retention time=9.033 min). Stereochemical assignment of (1 S) at the THIQ is absolute based on starting materials

Compound 2016: LCMS: (M+H)⁺=395; purity=100% (214 nm); retention time=2.017 min. CP Method C

¹H NMR (400 MHz, CD₃OD) δ 7.27-7.17 (m, 5H), 7.09-7.00 (m, 3H), 6.30 (s, 1H), 4.90-4.83 (m, 1H), 4.05-3.94 (m, 1H), 3.31-3.25 (m, 2H), 3.17-3.12 (m, 1H), 3.00-2.90 (m, 1H), 2.87-2.75 (m, 1H), 2.31 (s, 3H), 2.17-2.05 (m, 1H), 2.00-1.81 (m, 3H), 1.80-1.74 (m, 1H), 1.65-1.56 (m, 1H), 1.55-1.47 (m, 1H).

Chiral SFC: CO₂/MeOH containing 0.2% ammonia over CHIRALPAK® IG column (4.6*250 mm 5 μm), retention time=1.64 min), 100% ee.

Compound 2017: LCMS: (M+H)⁺=395; purity=97% (214 nm); retention time=2.019 min. CP Method C

¹H NMR (400 MHz, CD₃OD) δ 7.29-7.16 (m, 5H), 7.10-6.99 (m, 3H), 6.41-6.16 (br, 1H), 4.89-4.81 (m, 1H), 3.98 (dt, J=13.2, 4.8 Hz, 1H), 3.42-3.24 (br, 2H), 3.18-3.12 (m, 1H), 2.99-2.89 (m, 1H), 2.88-2.76 (m, 1H), 2.32 (s, 3H), 2.16-2.02 (m, 1H), 1.98-1.82 (m, 3H), 1.75 (t, J=14.8 Hz, 1H), 1.65-1.48 (m, 3H).

Chiral SFC: CO₂/MeOH containing 0.2% ammonia over CHIRALPAK® IG column (4.6*250 mm 5 μm), retention time=1.70 min), 100% ee.

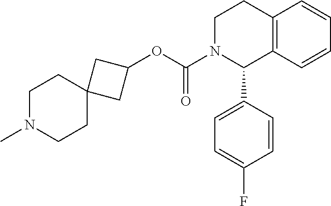 [SMILES: CN1CCC2(CC1)CC(OC(=O)N1CCc3ccccc3[C@@H]1c1ccc(F)cc1)C2]

Compound 2018: LCMS: (M+H)=409; purity=100% (214 nm); retention time=1.975 min. CP Method C

¹H NMR (400 MHz, CD₃OD) δ 7.30-7.16 (m, 5H), 7.09-6.99 (m, 3H), 6.38-6.24 (m, 1H), 5.08-4.99 (m, 1H), 4.05-3.97 (m, 1H), 3.39-3.37 (m, 1H), 3.22-3.02 (m, 4H), 3.01-2.91 (m, 1H), 2.89-2.83 (m, 1H), 2.79 (s, 3H), 2.49-2.41 (m, 2H), 2.06-1.93 (m, 2H), 1.91-1.79 (m, 4H).

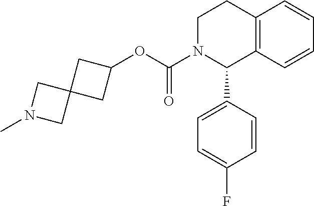 [SMILES: CN1CC2(CC(OC(=O)N3CCc4ccccc4[C@@H]3c3ccc(F)cc3)C2)C1]

Compound 2019: LCMS: (M+H)⁺=381; purity=100% (214 nm); retention time=1.573 min. CP Method B

¹H NMR (400 MHz, CDCL₃) δ 7.28-6.94 (m, 8H), 6.37-6.30 (m, 1H), 4.91 (t, J=6.4 Hz, 1H), 4.08-3.92 (m, 5H), 3.20 (td, J=13.6, 3.6 Hz, 1H), 2.98-2.93 (m, 1H), 2.76-2.73 (m, 3H), 2.70 (s, 3H), 2.38-2.33 (m, 2H).

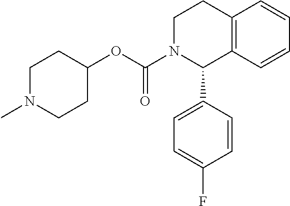 [SMILES: CN1CCC(OC(=O)N2CCc3ccccc3[C@@H]2c2ccc(F)cc2)CC1]

Compound 2020 LCMS: (M+H)⁺=369.1; purity=100% (214 nm); retention time=1.531 min. CP Method C

¹H NMR (400 MHz, DMSO-d₆) δ 8.20 (s, 1H), 7.23-7.13 (m, 8H), 6.23 (s, 1H), 4.69 (s, 1H), 3.87 (m, 1H), 3.32 (s, 1H), 2.89-2.67 (m, 3H), 2.50 (m, 3H), 2.30 (s, 3H), 1.88-1.86 (m, 2H), 1.69 (m, 1H).

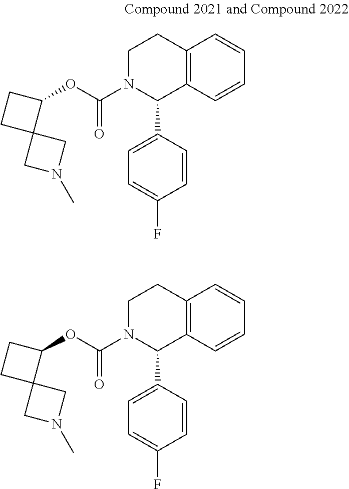 [SMILES: CN1CC2(CC[C@@H]2OC(=O)N2CCc3ccccc3[C@@H]2c2ccc(F)cc2)C1 ; CN1CC2(CC[C@H]2OC(=O)N2CCc3ccccc3[C@@H]2c2ccc(F)cc2)C1]

The diastereomers were separated by chiral SFC eluting with CO₂/MeOH containing 0.2% methanolic ammonia over an EnantioPak® AS column (4.6*100 mm 5 μm) to give compound 2021 (retention time=1.29 min) and compound 2022 (retention time=2.41 min). Stereochemical assignment of (1S) at THIQ is absolute based on starting materials, stereochemical assignment at the spirocyclic azetidine is assigned based solely on chromatographic elution order.

Compound 2021: LCMS: (M+H)⁺=381; purity=100% (214 nm); retention time=1.621 min. CP Method C

¹H NMR (400 MHz, CD₃OD) δ 7.38-7.17 (m, 5H), 7.13-6.98 (m, 3H), 6.44-6.22 (m, 1H), 4.11-4.03 (m, 1H), 3.64-3.38 (m, 2H), 3.37 (s, 3H), 3.32-3.24 (m, 1H), 3.18-2.94 (m, 1H), 2.93-2.81 (m, 1H), 2.32-2.18 (m, 4H), 2.14-2.01 (m, 1H), 1.98-1.81 (m, 2H).

Chiral SFC: CO₂/MeOH containing 0.2% ammonia over CHIRALPAK® IG column (4.6*100 mm 5 μm), retention time=1.3 min), 100% ee.

Compound 2022: LCMS: (M+H)⁺=381; purity=100% (214 nm); retention time=1.600 min. CP Method C

¹H NMR (400 MHz, CD₃OD) δ 7.32-7.17 (m, 5H), 7.16-7.02 (m, 3H), 6.45-6.35 (m, 1H), 4.11-4.03 (m, 1H), 3.52-3.39 (m, 2H), 3.38-3.35 (m, 2H), 3.31-3.24 (m, 2H), 3.08-2.92 (m, 1H), 2.91-2.81 (m, 1H), 2.26 (s, 4H), 2.11-1.98 (m, 1H), 1.94-1.81 (m, 2H).

Chiral SFC: CO₂/MeOH containing 0.2% ammonia over CHIRALPAK® IG column (4.6*100 mm 5 μm), retention time=2.28 min), 100% ee.

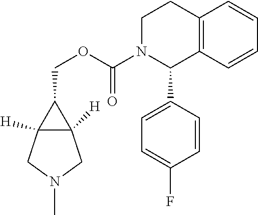 [SMILES: CN1C[C@@H]2[C@@H](COC(=O)N3CCc4ccccc4[C@@H]3c3ccc(F)cc3)[C@@H]2C1]

Compound 2023: LCMS: (M+H)⁺=381; purity=100% (214 nm); Retention time=1.407 min. CP Method A

¹H NMR (400 MHz, CDCl₃) δ 7.26-7.12 (m, 4H), 7.02 (d, J=7.5 Hz, 3H), 6.32 (d, J=39.7 Hz, 1H), 4.03 (d, J=6.6 Hz, 2H), 3.74-3.51 (m, 3H), 3.35-3.15 (m, 2H), 3.05-2.91 (m, s, 2H), 2.78 (d, J=16.0 Hz, 1H), 2.73 (s, 3H), 2.09-1.95 (m, s, 1H), 1.46-1.37 (m, 1H), 1.31-1.22 (m, s, 1H).

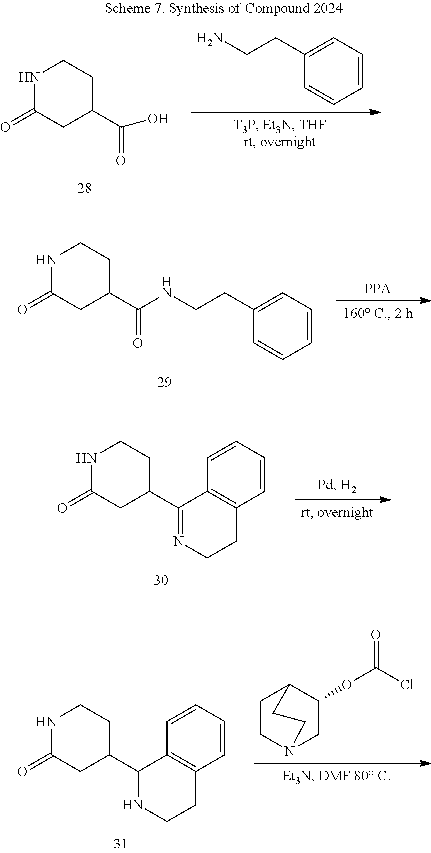

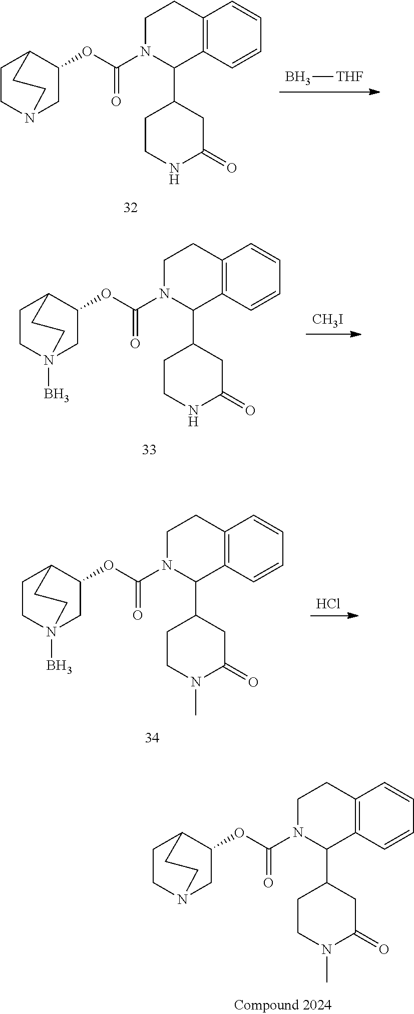

Step 1: To a solution of 2-oxopiperidine-4-carboxylic acid 28 (2.86 g, 20 mmol) in THF (50 ml) was added triethylamine (6.1 g, 60 mmol), 2-phenylethanamine (2.54 g, 21 mmol) and propylphosphonic anhydride (T3P, 13.4 g, 21 mmol). The mixture was stirred at room temperature overnight. The mixture was quenched with water (40 mL) and extracted with three 50 mL portions of dichloromethane/methanol. The combined extracts were dried with Na₂SO₄. The solvent was evaporated to give 2-oxo-N-phenethylpiperidine-4-carboxamide 29 (3.9 g) as a white solid.

LCMS: (M+H)=247; purity=58% (254 nm); retention time=1.163 min. CP Method E.

Step 2: To the 2-oxo-N-phenethylpiperidine-4-carboxamide 29 (3.9 g, 15.9 mmol) was added polyphosphoric acid (10 mL). The mixture was stirred overnight at 160° C. The mixture was cooled to 25° C. and quenched with ice water (100 mL), alkalized with NaOH (10% aq.) to pH=11 and extracted with three 100 mL portions of dichloromethane/methanol. The combined organic layers were washed with brine (100 mL), dried and concentrated in vacuo to give 4-(3,4-dihydroisoquinolin-1-yl)piperidin-2-one 30 (2.6 g) as a yellow solid.

LCMS: (M+H)=229; purity=72% (254 nm); retention time=1.020 min, 1.093 min. CP Method C1.

Step 3: A suspension of 4-(3,4-dihydroisoquinolin-1-yl)piperidin-2-one 30 (2.6 g, 11.4 mmol) and 5% palladium on carbon (0.13 g) in EtOH (40 mL) was stirred under a hydrogen atmosphere. After of the reaction was complete, the mixture was filtered and the solvent was evaporated to give 4-(1,2,3,4-tetrahydroisoquinolin-1-yl)piperidin-2-one 31 (2.4 g) as a yellow oil.

LCMS: (M+H)=231; purity=89% (214 nm); retention time=0.996 min. CP Method E.

Step 4: To a solution of 14-(1,2,3,4-tetrahydroisoquinolin-1-yl)piperidin-2-one 31 (460 mg, 2.0 mmol) in DMF (5 mL) was added (S)-quinuclidin-3-yl carbonochloridate (570 mg, 3 mmol) and TEA (606 mg, 6.0 mmol). The mixture was stirred at 80° C. for 2 h. The mixture was cooled to 25° C. and water was added (20 mL). The mixture was extracted with three 20 mL portions of dichloromethane/methanol (20/1). The combined organic layers were washed with brine (20 mL), dried and concentrated in vacuo to give crude product. The crude product was purified by liquid preparation CP Method (Mobile Phase: A:H₂O (10 mM NH₄HCO₃) B:MeCN Gradient: 5%-95% B in 1.2 min Flow Rate: 2.0 mL/min Column: XBridge C18 50*4.6 mm, 3.5 umi oven temperature: 40° C. UV214, MASS100-1000) to give (S)-quinuclidin-3-yl 1-(2-oxopiperidin-4-yl)-3,4-dihydroisoquinoline-2(1H)-carboxylate 32 (182 mg) as a white solid.

LCMS: (M+H)=384; purity=95% (214 nm); retention time=1.210 min. CP Method A1.

Step 5: The solution of (S)-quinuclidin-3-yl 1-(2-oxopiperidin-4-yl)-3,4-dihydroisoquinoline-2(1H)-carboxylate 32 (182 mg, 0.475 mmol) in tetrahydrofuran (5 mL) was cooled to −78° C., and then borane-tetrahydrofuran complex (0.95 mL, 0.95 mmol, 1M) was added dropwise. The mixture was stirred at rt for 3 h, diluted with 20 mL of water and extracted by three 30 mL portions of dichloromethane. The combined organic layers were dried with Na₂SO₄ and concentrated to give borane complex 33 (100 mg) as a yellow oil.

LCMS: (M−H)=396; purity=81% (214 nm); retention time=1.531 min. CP Method E.

Step 6: The solution of 33 (100 mg, 0.25 mmol) in DMF (2 mL) was cooled to 0° C., and then sodium hydride (13 mg, 0.312 mmol) was added in portions. The mixture was stirred at rt for 0.5 h and then iodomethane (0.017 mL, 0.286 mmol, 2.28 g/mL) was added. The mixture was stirred at room temperature for 2 h, quenched with water (10 mL), and extracted with three 30 mL portions of dichloromethane/methanol (20/1). The combined organic layers were dried with Na₂SO₄. The solvent was evaporated to give 34 (45 mg) as a yellow oil.

LCMS: (M+H)=412; purity=72% (254 nm); retention time=1.332 min. CP Method C1.

Step 7: To a solution of 34 (45 mg, 0.11 mmol) in THF (1 mL) was added hydrochloric acid (1 mL, 12 mmol, 12M). The mixture was stirred at room temperature overnight. The mixture was alkalized with NaOH (10% aq.) to pH=8 and extracted with three 30 mL portions of dichloromethane/methanol (20/1). The combined organic layers were dried and concentrated in vacuo to give crude product. The crude product was purified by HPLC CP Method (Mobile Phase: A: H₂O (10 mM NH₄HCO₃) B: MeCN Gradient: 5%-95% B in 1.2 min, Flow Rate: 2.0 mL/min Column: XBridge C18 50*4.6 mm, 3.5 μmi, oven temperature: 40° C. UV214, MASS:100-1000) to give (S)-quinuclidin-3-yl 1-(1-methyl-2-oxopiperidin-4-yl)-3,4-dihydroisoquinoline-2(1H)-carboxylate, compound 2024.

Compound 2024: LCMS: (M+H)⁺=398; purity=100% (214 nm); retention time=1.347 min. CP Method C

¹H NMR (400 MHz, CD₃OD) δ 7.35-7.27 (m, 1H), 7.26-7.18 (m, 3H), 5.15-5.09 (t, J=6.4 Hz, 1H), 4.34-4.27 (m, 1H), 3.73-3.64 (m, 1H), 3.41-3.34 (m, 1H), 3.31-3.16 (m, 5H), 3.15-3.04 (m, 1H), 3.03-2.92 (m, 1H), 2.90-2.81 (m, 3H), 2.79-2.70 (m, 4H), 2.42-2.27 (m, 2H), 2.24-2.00 (m, 2H), 1.99-1.83 (m, 2H), 1.43-1.28 (m, 2H).

The following compounds were prepared using General Procedure GP-4.

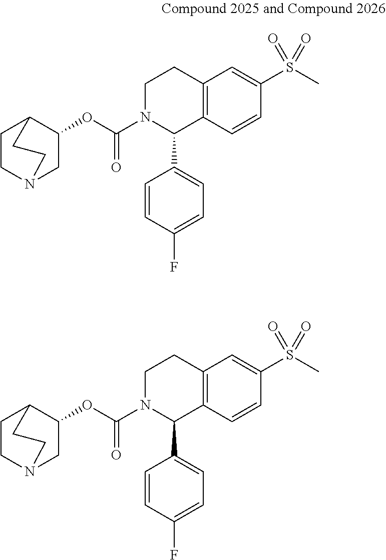 [SMILES: CS(=O)(=O)c1ccc2c(c1)CCN(C(=O)O[C@@H]1CN3CCC1CC3)[C@H]2c1ccc(F)cc1 ; CS(=O)(=O)c1ccc2c(c1)CCN(C(=O)O[C@@H]1CN3CCC1CC3)[C@@H]2c1ccc(F)cc1]

The diastereomers were separated by chiral SFC eluting with EtOH containing 0.2% ammonia in methanol over an EnantioPak® IG column (4.6*250 mm 5 μm) to give compound 2025 (retention time=19.134 min) and compound 2026 (retention time=24.694 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2025: LCMS: (M+H)⁺=459; purity=100% (214 nm); Retention time=1.344 min. CP Method A

¹H NMR (400 MHz, DMSO-d₆) δ 7.83 (s, 1H), 7.73 (d, J=8.4 Hz, 1H), 7.47-7.40 (br, s, 1H), 7.20 (dd, J=11.6, 8.8 Hz, 4H), 6.38 (s, 1H), 4.68-4.60 (br, s, 1H), 4.01-3.90 (m, 1H), 3.22 (s, 4H), 3.11-3.03 (m, 2H), 3.02-2.92 (br, s, 2H), 2.75-2.66 (m, 2H), 2.63-2.54 (m, 2H), 1.95-1.86 (br, s, 1H), 1.63-1.54 (br, s, 1H), 1.48-1.44 (br, s, 1H), 1.27-1.16 (br, s, 2H).

Chiral SFC: n-hexane (containing 0.1% DEA)/EtOH (containing 0.1% DEA) over an ENANTIOPAK® IG column (4.6*250 mm 5 μm), retention time=19.134 min), 100% ee.

Compound 2026: LCMS: (M+H)⁺=459; purity=100% (214 nm); Retention time=1.367 min. CP Method A

¹H NMR (400 MHz, DMSO-d₆) δ 8.37 (s, 1H), 7.91-7.65 (m, 2H), 7.54-7.42 (m, 1H), 7.41-7.05 (m, 4H), 6.38 (s, 1H), 4.98-4.77 (br, s, 1H), 3.46-3.31 (br, s, 2H), 3.21 (s, 3H), 3.11-2.84 (m, 7H), 2.81-2.67 (br, s, 1H), 2.25-2.10 (br, s, 1H), 2.01-1.53 (m, 4H).

Chiral SFC: CO₂/MeOH containing 0.2% ammonia in methanol over an ENANTIOPAK® AS column (4.6*100 mm 5 μm), retention time=24.694 min), 95% ee.

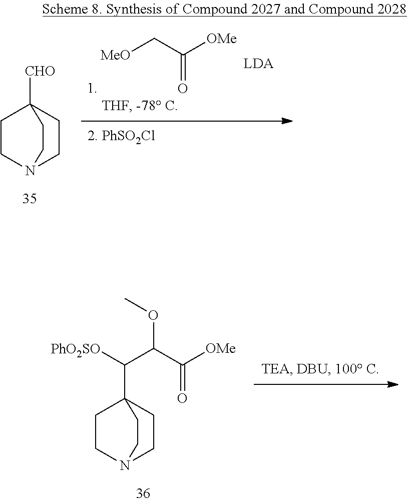

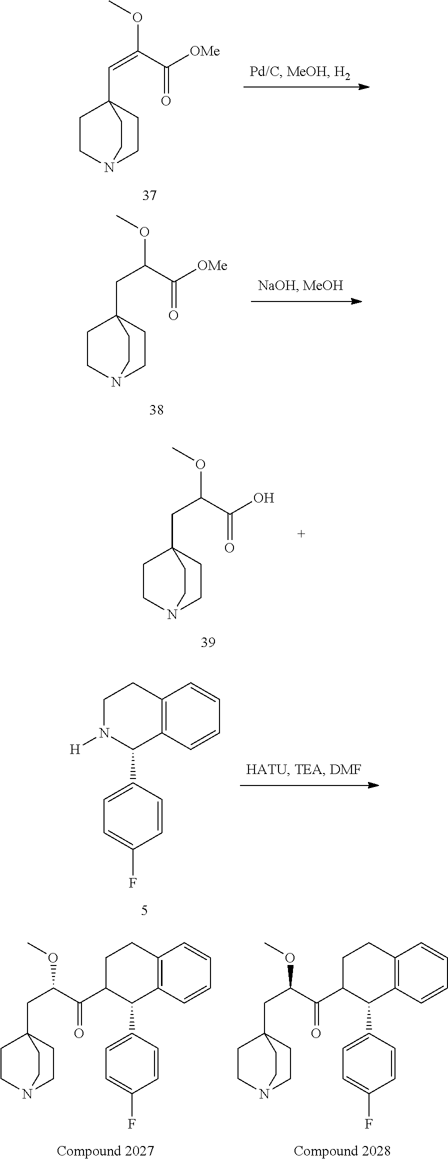 [SMILES: COC(=O)/C(=C\C12CCN(CC1)CC2)OC ; COC(=O)C(CC12CCN(CC1)CC2)OC ; COC(CC12CCN(CC1)CC2)C(=O)O ; Fc1ccc([C@@H]2NCCc3ccccc32)cc1 ; CO[C@@H](CC12CCN(CC1)CC2)C(=O)C1CCc2ccccc2[C@@H]1c1ccc(F)cc1 ; CO[C@H](CC12CCN(CC1)CC2)C(=O)C1CCc2ccccc2[C@@H]1c1ccc(F)cc1]

Step 1: LDA (3 mL, 5.9 mmol) was added to a solution of methyl 2-methoxyacetate (3 mL, 5.9 mmol) in THF (15 mL) at −78° C. After stirring for 30 min, a solution of quinuclidine-4-carbaldehyde (700 mg, 5 mmol) in THF (5 mL) was added dropwise. The mixture was stirred for 2 hours at −78° C. Then PhSO₂Cl (0.64 mL, 5 mmol) was added and the resulting mixture was allowed to warm to room temperature and stirred overnight. The reaction was quenched with brine and extracted with 3×60 mL portions of ethyl acetate. The combined organic layers were dried over Na₂SO₄ and concentrated in vacuo to give 400 mg of methyl 2-methoxy-3-(phenylsulfonyloxy)-3-(quinuclidin-4-yl)propanoate 36 as a yellow oil.

LCMS: (M+H)⁺=384; purity=72% (214 nm); retention time=1.602 min, CP Method E.

Step 2: The methyl 2-methoxy-3-(phenylsulfonyloxy)-3-(quinuclidin-4-yl)propanoate from step 1 (400 mg, 1.0 mmol) was mixed with TEA (2.3 mL) and DBU (0.6 mL) and heated under reflux for 3 hours. After cooling to room temperature, the mixture was concentrated in vacuo to give 100 mg of methyl 2-methoxy-3-(quinuclidin-4-yl)acrylate 37 as a yellow oil.

LCMS: (M+H)⁺=226; purity=70% (214 nm); retention time=1.590 min, CP Method E.

Step 3: Pd/C (40 mg) was added to a solution of methyl 2-methoxy-3-(quinuclidin-4-yl)acrylate 37 (100 mg, 0.4 mmol). The mixture was stirred overnight under H₂ atmosphere. The mixture was filtered and the filtrate was concentrated to give 100 mg of methyl 2-methoxy-3-(quinuclidin-4-yl)propanoate 38 as yellow oil.

LCMS: (M+H)⁺=228; retention time=1.560 min, CP Method E.

Step 4: A mixture of methyl 2-methoxy-3-(quinuclidin-4-yl)propanoate 38 (100 mg, 0.4 mmol) in NaOH (5N aq.) (1 mL), MeOH (1 mL) and THF (2 mL) was stirred at room temperature for 2 hours. The solvent was removed under vacuum and the pH was adjusted to ˜5 with 1N aqueous HCl. The mixture was concentrated. MeOH (5 mL) was added. The mixture was filtered and the filtrate was concentrated to give 60 mg of methyl 2-methoxy-3-(quinuclidin-4-yl)propanoic acid 39 as a yellow solid.

LCMS: (M+H)⁺=214; retention time=0.801 min, CP Method E.

Step 5: TEA (49 μL, 0.3 mmol) was added to a mixture of 2-methoxy-3-(quinuclidin-4-yl)propanoic acid 39 (52 mg, 0.2 mmol), (S)-1-(4-fluorophenyl)-1,2,3,4-tetrahydroisoquinoline 5 (40 mg, 0.2 mmol) and HATU (80 mg, 0.2 mmol) in DMF (1 mL). The mixture was stirred at room temperature for 2 hours. The crude was purified by prep-HPLC to give the diastereomers as a mixture.

The diastereomers were separated by chiral SFC eluting with CO₂/MeOH containing 0.2 methanol ammonia over an EnantioPak® OZ column (20*250 mm 10 μm) to give Compound 2027 (retention time=2.97 min) and Compound 2028 (retention time=3.53 min). Stereochemical assignment at 1 position of the tetrahydroisoquinoline is absolute based on use of chiral starting materials (5). Stereochemical assignment of 2 position of the amide alkoxy is based on elution order and is assigned randomly.

Compound 2027: LCMS: (M+H)⁺=423; purity=96% (214 nm); retention time=1.413 min. CP Method A1

¹H NMR (400 MHz, CD₃OD) δ 7.28 (d, J=4.0 Hz, 2H), 7.27-7.21 (m, 3H), 7.04 (dd, J=18.0, 8.8 Hz, 3H), 6.80 (s, 1H), 4.34 (dd, J=9.6, 2.8 Hz, 1H), 4.16 (ddd, J=13.2, 4.8, 2.4 Hz, 1H), 3.54-3.44 (m, 1H), 3.29 (s, 3H), 3.12-3.00 (m, 1H), 2.99-2.86 (m, 7H), 1.67 (dd, J=14.8, 9.2 Hz, 1H), 1.62-1.53 (m, 5H), 1.40 (dd, J=14.8, 2.8 Hz, 1H), 1.34-1.30 (m, 1H).

Chiral SFC: MeOH (0.2% Methanol Ammonia) over an ENANTIOPAK® IG column (4.6*100 mm 5 μm), retention time=3.70 min), 94% ee.

Compound 2028: LCMS: (M+H)⁺=423; purity=98% (214 nm); retention time=1.548 min. CP Method C1

¹H NMR (400 MHz, CD₃OD) δ 7.28 (d, J=4.0 Hz, 2H), 7.25-7.18 (m, 3H), 7.07-7.00 (m, 3H), 6.87 (s, 1H), 4.35 (dd, J=9.6, 2.4 Hz, 1H), 4.16-4.08 (m, 1H), 3.46-3.38 (m, 1H), 3.21 (s, 3H), 3.13-3.03 (m, 1H), 2.98-2.87 (m, 7H), 1.64 (dd, J=14.8, 9.2 Hz, 1H), 1.60-1.50 (m, 5H), 1.39 (dd, J=14.8, 2.4 Hz, 1H), 1.34-1.30 (m, 1H).

Chiral SFC: MeOH (0.2% Methanol Ammonia) over an ENANTIOPAK® IG column (4.6*100 mm 5 μm), retention time=3.72 min), 95% ee.

The following compounds were prepared using General Procedure GP-4.

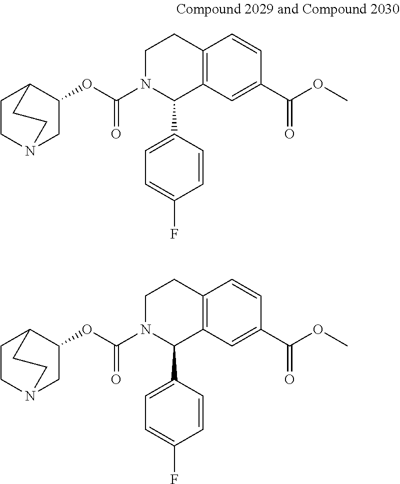

The diastereomers were separated by chiral SFC eluting with n-hexane containing 0.1% DEA/EtOH containing 0.1% DEA over an EnantioPak® IC column (20*250 mm 10 μm) to give compound 2029 (retention time=9.185 min) and compound 2030 (retention time=7.471 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2029: LCMS: (M+H)⁺=439; purity=99% (214 nm); retention time=1.450 min. CP Method A1

¹H NMR (400 MHz, CD₃OD) δ 7.89 (d, J=8.0 Hz, 1H), 7.75 (s, 1H), 7.38 (d, J=8.0 Hz, 1H), 7.26 (s, 2H), 7.07 (t, J=8.4 Hz, 2H), 6.41 (s, 1H), 4.86-4.79 (m, 1H), 4.09 (dt, J=13.2, 4.8 Hz, 1H), 3.87 (s, 3H), 3.48-3.36 (br, 1H), 3.27 (dd, J=14.4, 8.4 Hz, 1H), 3.09-2.70 (m, 7H), 2.12-2.08 (m, 1H), 1.96-1.72 (m, 2H), 1.70-1.60 (m, 1H), 1.52 (s, 1H).

Chiral SFC: MeOH (0.2% ammonia) over an ENANTIOPAK® IG column (4.6*100 mm 5 μm), retention time=2.95 min), 100% ee.

Compound 2030: LCMS: (M+H)⁺=601; purity=100% (214 nm); retention time=1.456 min. CP Method A1

¹H NMR (400 MHz, CD₃OD) δ 7.90 (d, J=8.4 Hz, 1H), 7.77 (s, 1H), 7.39 (d, J=8.0 Hz, 1H), 7.26 (s, 2H), 7.07 (s, 2H), 6.41 (s, 1H), 4.86-4.79 (m, 1H), 4.05 (s, 1H), 3.87 (s, 3H), 3.50-3.36 (br, 1H), 3.24 (dd, J=14.4, 8.4 Hz, 1H), 3.10-2.66 (m, 7H), 2.10-2.04 (m, 1H), 1.88 (s, 1H), 1.82-1.72 (m, 1H), 1.70-1.60 (m, 1H), 1.58-1.46 (m, 1H).

Chiral SFC: MeOH (0.2% ammonia) over an ENANTIOPAK® IG column (4.6*100 mm 5 μm), retention time=3.83 min), 96% ee.

The following compounds were prepared using General Procedure GP-4.

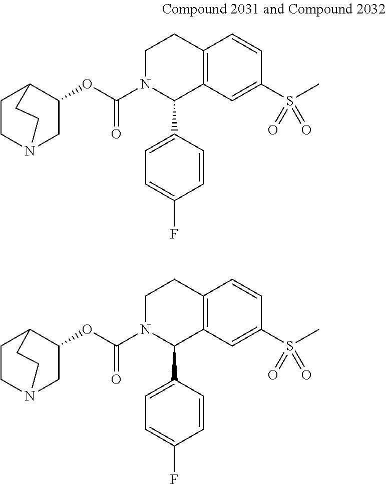

The diastereomers were separated by chiral SFC eluting with EtOH containing 0.2% methanolic ammonia over an EnantioPak® IG column (4.6*250 mm 5 μm) to give compound 2031 (retention time=29.368 min) and compound 2032 (retention time=36.186 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2031: LCMS: (M+H)⁺=459; purity=100% (214 nm); Retention time=1.524 min. CP Method C

¹H NMR (400 MHz, DMSO-d₆) δ 7.81-7.70 (m, 2H), 7.54 (d, J=8.1 Hz, 1H), 7.37-7.13 (m, 4H), 6.44 (s, 1H), 4.64 (s, 1H), 3.99-3.89 (m, 1H), 3.17 (s, 3H), 3.13-3.04 (m, 1H), 3.02-2.93 (m, 2H), 2.80-2.53 (m, 5H), 1.96-1.85 (m, 1H), 1.75-1.54 (m, 2H), 1.53-1.39 (m, 2H), 1.28-1.24 (m, 1H).

Chiral SFC: MeOH (0.2% methanol ammonia) over an ENANTIOPAK® IG column (4.6*250 mm 5 μm), retention time=29.331 min), 100% ee.

Compound 2032: LCMS: (M+H)⁺=459; purity=100% (214 nm); Retention time=1.536 min. CP Method C

¹H NMR (400 MHz, DMSO-d₆) δ 7.79 (d, J=7.5 Hz, 2H), 7.53 (d, J=8.1 Hz, 1H), 7.40-7.13 (m, 4H), 6.43 (s, 1H), 4.70-4.61 (m, 1H), 3.96-3.86 (m, 1H), 3.18 (s, 3H), 3.11-3.03 (m, 1H), 3.02-2.87 (m, 2H), 2.79-2.53 (m, 5H), 1.95-1.88 (m, 1H), 1.81-1.68 (m, 1H), 1.64-1.54 (m, 1H), 1.52-1.42 (m, 1H), 1.38-1.28 (m, 2H).

Chiral SFC: MeOH (0.2% methanol ammonia) over an ENANTIOPAK® IG column (4.6*250 mm 5 μm), retention time=35.477 min), 100% ee.

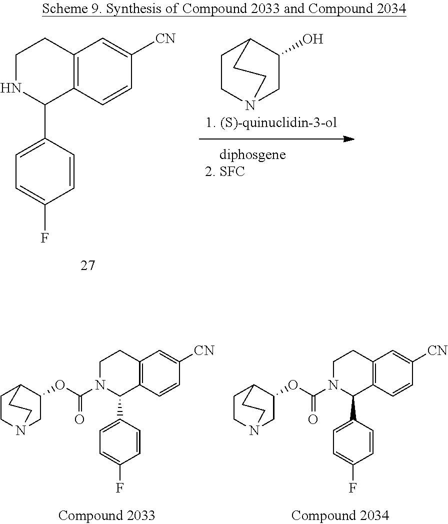 [SMILES: N#Cc1ccc2c(c1)CCNC2c1ccc(F)cc1 ; O[C@@H]1CN2CCC1CC2 ; N#Cc1ccc2c(c1)CCN(C(=O)O[C@@H]1CN3CCC1CC3)[C@H]2c1ccc(F)cc1 ; N#Cc1ccc2c(c1)CCN(C(=O)O[C@@H]1CN3CCC1CC3)[C@@H]2c1ccc(F)cc1]

Step 1: step A: To a solution of (S)-quinuclidin-3-ol (76.32 mg, 1.03 mmol) in MeCN (9 mL) was added diphosgene (278 mg, 1.72 mmol) at rt temperature for 2 h. Then the solution was concentrated under reduced pressure to give the white solid.

step B: To a solution of solution of 1-(4-fluorophenyl)-1,2,3,4-tetrahydroisoquinoline-6-carbonitrile 27 (100 mg, 0.4 mmol) in DMF (2 mL) was added the crude compound of step A at 80° C. overnight to give the expected compound.

LCMS: (M+H)⁺=406.1 (214 nm); retention time=1.50 min. CP Method D

The diastereomers were separated by chiral SFC eluting with MeOH (0.2% methanol ammonia) over an EnantioPak® OZ (4.6*100*5 um) to give Compound 2033 (retention time=4.46 min) and Compound 2034 (retention time=2.72 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2033: LCMS: (M+H)⁺=406.1; purity=100% (214 nm); retention time=1.491 min. CP Method C

¹H NMR (400 MHz, CD₃OD) δ 7.67 (s, 1H), 7.56 (d, J=8 Hz, 1H), 7.29 (d, J=8 Hz, 3H), 7.08 (t, J=8.8 HZ, 2H), 6.14 (s, 1H), 4.88 (s, 1H), 4.13-4.07 (m, 1H), 3.40-3.36 (m, 1H), 3.07-2.87 (m, 8H), 2.16 (s, 1H), 1.89-1.70 (m, 3H), 1.64-1.56 (m, 1H)

Chiral SFC: MeOH (0.2% methanol ammonia) over an ENANTIOPAK® IG column (4.6*100 mm 5 μm), retention time=2.91 min), 100% ee.

Compound 2034: LCMS: (M+H)⁺=406.1; purity=100% (214 nm); retention time=1.505 min. CP Method C

¹H NMR (400 MHz, CD₃OD) δ 7.67 (s, 1H), 7.58-7.55 (dd, J=8 Hz; 1.2 Hz, 1H), 7.31-7.28 (m, 3H), 7.10-7.06 (m, 2H), 6.39 (s, 1H), 4.92-4.81 (m, 1H), 4.63 (s, 1H), 4.06-1.05 (m, 1H), 3.30-3.13 (m, 1H), 3.01-2.78 (m, 7H), 2.08-2.07 (m, 1H), 1.79-1.77 (m, 2H), 1.66-1.65 (m, 1H), 1.52-1.51 (m, 1H)

Chiral SFC: MeOH (0.2% methanol ammonia) over an ENANTIOPAK® IG column (4.6*100 mm 5 μm), retention time=3.78 min), 99% ee.

The following compounds were prepared analogously:

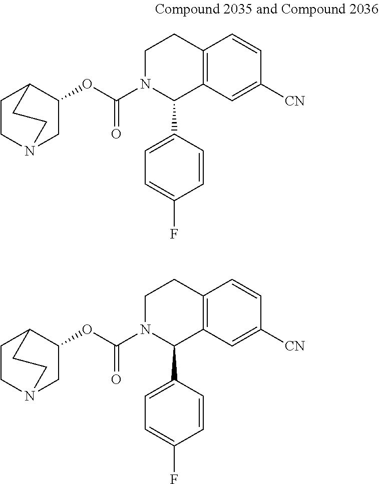

The diastereomers were separated by chiral SFC eluting with EtOH containing 0.2% methanol ammonia over an EnantioPak® IG column (4.6*250 mm 5 μm) to give Compound 2035 (retention time=1.97 min) and Compound 2036 (retention time=3.62 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2035: LCMS: (M+H)⁺=406.0; purity=100% (214 nm); retention time=1.547 min. CP Method C

¹H NMR (400 MHz, CD₃OD) δ 7.61 (d, J=8.4 Hz, 1H), 7.53-7.45 (m, 2H) 7.27 (s, 2H), 7.09 (t, J=8.8 Hz, 1H), 6.42 (s, 1H), 4.85-4.83 (m, 1H), 4.11-4.05 (m, 1H), 3.09-2.99 (m, 3H), 2.90-2.78 (m, 6H), 2.10-2.04 (m, 2H), 1.80-1.79 (m, 1H), 1.64-1.66 (m, 1H), 1.53-1.51 (m, 1H).

Chiral SFC: MeOH (0.2% methanol ammonia) over an ENANTIOPAK® IG column (4.6*100 mm 5 μm), retention time=1.97 min), 100% ee.

Compound 2036: LCMS: (M+H)⁺=406.2; purity=100% (214 nm); retention time=1.444 min. CP Method C

¹H NMR (400 MHz, CD₃OD) δ 7.63-7.55 (m, 2H), 7.46 (d, J=8 Hz, 1H), 7.30-7.25 (m, 2H), 7.11-7.09 (m, 2H), 6.43 (s, 1H), 4.64 (s, 1H), 3.95-3.90 (m, 1H), 3.07-2.94 (m, 9H), 2.21-2.19 (m, 1H), 2.05-1.75 (m, 2H), 1.65-1.61 (m, 2H).

Chiral SFC: MeOH (0.2% methanol ammonia) over an ENANTIOPAK® IG column (4.6*100 mm 5 μm), retention time=3.62 min), 100% ee.

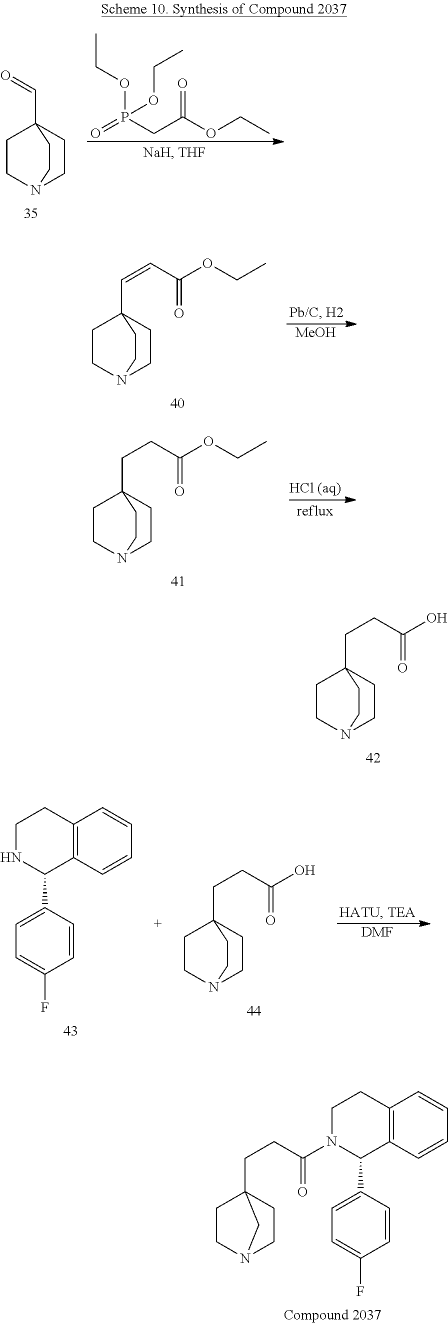

Step 1: To a solution of quinuclidine-4-carbaldehyde 35 (280 mg, 2 mmol) in THF (5 mL) was added NaH (60%, 120 mg, 3 mmol) and ethyl 2-(diethoxyphosphoryl)acetate (492 mg, 2.2 mmol). The mixture was stirred at 35° C. for 1.5 h. To the mixture was added 1N NH₄Cl (10 mL) and it was extracted with three 10 mL portions of DCM. The organic phase was washed with brine, dried over Na₂SO₄, and concentrated under reduced pressure. The residue was purified by prep-HPLC to obtain (Z)-ethyl 3-(quinuclidin-4-yl) acrylate 40 (310 mg) as a white solid.

LCMS: (M+H)⁺=210; retention time=1.035 min. CP Method C

The solution of (Z)-ethyl 3-(quinuclidin-4-yl)acrylate (209 mg, 1 mmol) in THF (5 mL) was stirred under 1 atm H₂. The mixture was stirred at 35° C. for 3.5 h. The mixture was filtered and the organic phase was concentrated under reduced pressure to give the desired product ethyl 3-(quinuclidin-4-yl) propanoate 41 (200 mg) as a yellow oil.

LCMS: (M+H)⁺=212; retention time=1.107 min. CP Method C

Step 3: To a solution of ethyl 3-(quinuclidin-4-yl) propanoate 41 (200 mg, 0.95 mmol) in water (2 mL) was added dropwise 10 mL of concentrated hydrochloric acid. The mixture was stirred at reflux for 2.5 h, and concentrated under reduced pressure to give the desired product 3-(quinuclidin-4-yl)propanoic acid (180 mg, yield 98.4%) as a yellow oil.

LCMS: (M+H)=184; retention time=1.215 min. CP Method C

Step 4: Compound 2037 was prepared following the General Procedure GP-5 for amide synthesis.

Compound 2037: LCMS: (M+H)⁺=393; (214 nm); retention time=1.675 min. CP Method C

¹H NMR (400 MHz, DMSO-d₆) δ 8.41 (s, 1H), 7.21 (d, J=47.5 Hz, 7H), 6.66 (s, 1H), 3.41 (d, J=47.4 Hz, 2H), 3.15 (s, 5H), 3.02-2.68 (m, 2H), 2.38 (d, J=11.2 Hz, 2H), 1.85 (d, J=105.6 Hz, 1H), 1.58 (d, J=31.4 Hz, 7H), 1.43-1.23 (m, 1H).

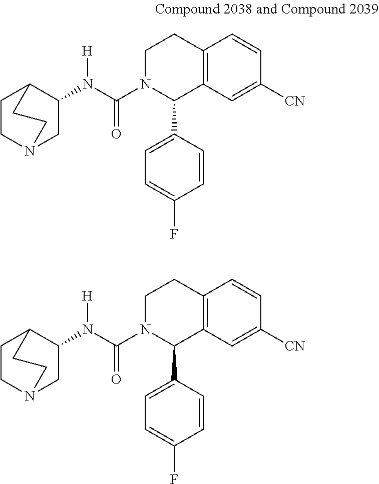

7-cyano-1-(4-fluorophenyl)-N—((S)-quinuclidin-3-yl)-3,4-dihydroisoquinoline-2(1H)-carboxamide was prepared following the General Procedure GP-1 using enantiomerically pure (S)-quinuclidine-3-amine.

The diastereomers were separated by chiral SFC eluting with C02/MeOH containing 0.2% methanolic ammonia over an EnantioPak® AD-H column (4.6*100 mm 5 μm) to give Compound 2038 (retention time=1.92 min) and Compound 2039 retention time=4.17 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2038: LCMS: (M+H)⁺=405.1; purity=100% (214 nm); retention time=1.587 min. CP Method C

¹H NMR (400 MHz, DMSO-d₆) δ 7.70 (m, 2H), 7.47 (d, J=8.4 Hz, 1H) 7.14 (d, J=7.2 Hz, 4H), 6.54 (s, 1H), 6.36 (d, J=6 Hz, 1H), 3.87-3.83 (m, 1H), 3.69 (m, 1H), 3.29-3.21 (m, 1H), 3.06-3.02 (m, 1H), 2.96-2.92 (m, 2H), 2.84-2.78 (m, 2H), 2.68-2.50 (m, 3H), 1.76-1.74 (m, 2H), 1.54-1.51 (m, 2H), 1.26-1.21 (m, 1H).

Chiral SFC: CO₂/MeOH containing 0.2% ammonia over CHIRALPAK® IG column (4.6*250 mm 5 μm), retention time=1.34 min), 100% ee.

Compound 2039: LCMS: (M+H)⁺=405.1; purity=100% (214 nm); retention time=1.599 min. CP Method C

¹H NMR (400 MHz, DMSO-d₆) δ 7.16-7.70 (m, 2H), 7.47 (d, J=8.4 Hz, 1H), 7.14 (d, J=7.6 Hz, 4H), 6.55 (s, 1H), 6.38 (d, J=5.6 Hz, 1H), 3.87-3.82 (m, 1H), 3.69-3.64 (m, 1H), 3.26-3.21 (m, 1H), 3.06-2.94 (m, 3H), 2.82-2.78 (m, 2H), 2.67-2.64 (m, 3H), 1.77 (s, 2H), 1.57-1.52 (m, 2H), 1.29-1.24 (m, 1H).

Chiral SFC: CO₂/MeOH containing 0.2% ammonia over CHIRALPAK® IG column (4.6*250 mm 5 μm), retention time=7.71 min), 100% ee.

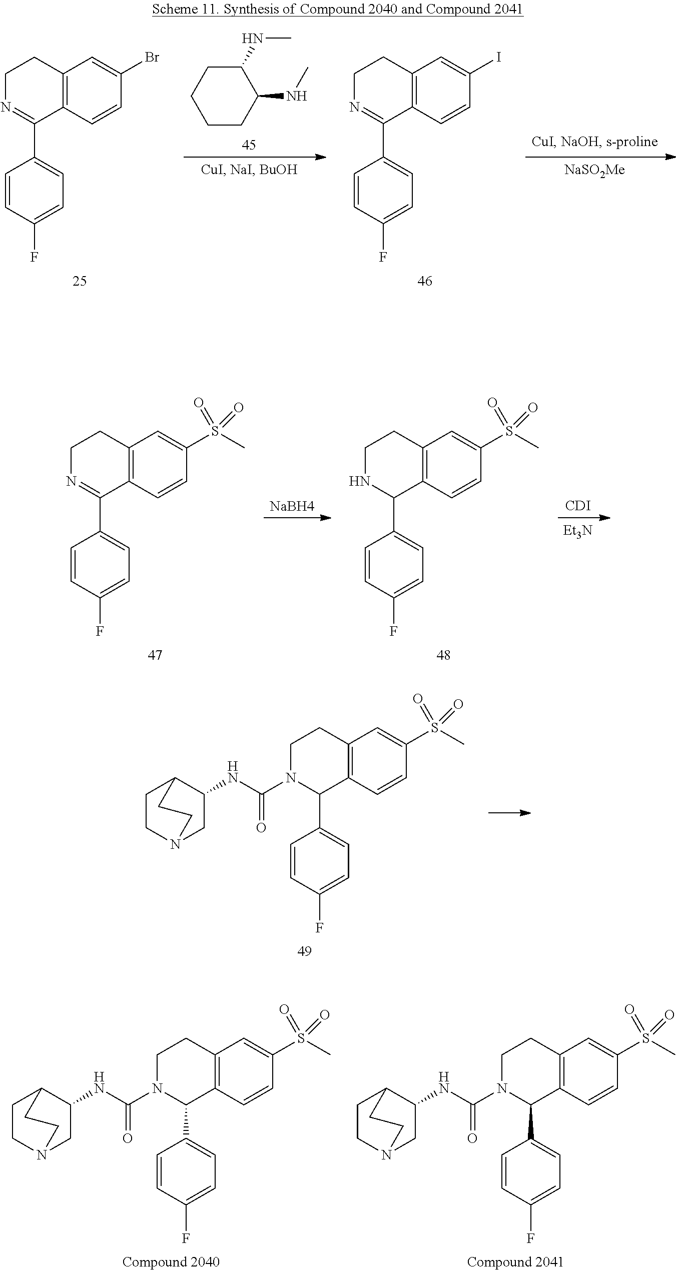

Step 1: 6-bromo-1-(4-fluorophenyl)-3, 4-dihydroisoquinoline 25 (1.52 g, 5 mmol), copper (1) iodide (95 mg, 0.5 mmol), and sodium iodide (1.5 mg, 10 mmol) were combined in a vial. Butan-1-ol (20 ml) and trans-(1R,2R)—N,N′-bismethyl-1,2-cyclohexanediamine 45 (142 mg, 1 mmol) were added and the resulting suspension was purged with argon (subsurface bubbling) for 5 min. The reaction mixture was heated to 130° C. and stirred for 22 hours. After cooling to room temperature, the reaction mixture was partitioned between ethyl acetate (60 mL) and brine (60 mL). The organic layer was washed with additional brine. The combined organics were dried over sodium sulfate, filtered and concentrated. The residue was purified by column chromatography on silica gel (solid SiO₂ loading) using an EtOAc/PE gradient to afford 1-(4-fluorophenyl)-6-iodo-3, 4-dihydroisoquinoline 46 (1 g).

LCMS: (M+H)⁺=352 (UV 214 nm); Retention time=1.40 min. CP Method E

Step 2: 1-(4-fluorophenyl)-6-iodo-3, 4-dihydroisoquinoline 46 (1 g, 2.8 mmol), copper (1) iodide (60 mg, 0.3 mmol), L-proline (68 mg, 0.6 mmol), sodium hydroxide (24 mg, 0.6 mmol), and methanesulfinic acid sodium salt (398 mg, 3.9 mmol) were combined in a vial. DMSO (10 ml) was added and the resulting suspension was purged with argon (subsurface bubbling) for 5 min. The reaction mixture was stirred at 95° C. for 16 h. The crude material was filtered through celite. The filtrate was concentrated and purified by preparative reverse phase HPLC, eluting with an acetonitrile/water gradient (with 0.05% TFA as a modifier), to afford 1-(4-fluorophenyl)-6-(methylsulfonyl)-3, 4-dihydroisoquinoline 47 (606 mg).

LCMS: (M+H)⁺=304 (UV 214 nm); Retention time=1.088 min. CP Method E

Step 3: To a mixture of 1-(4-fluorophenyl)-6-(methylsulfonyl)-3, 4-dihydroisoquinoline 47 (303 mg, 1 mmol) in MeOH (6 ml) was added NaBH₄ (152 mg, 4 mmol) under nitrogen atmosphere. The reaction mixture was stirred at 25° C. for 16 hr. The reaction mixture was concentrated and the residue was diluted with water (10 mL) and extracted with three 10 mL portions of ethyl acetate. The combined organic layers were washed with brine (20 mL), dried and concentrated to obtain 1-(4-fluorophenyl)-6-(methylsulfonyl)-1,2,3,4-tetrahydroisoquinoline 48 (220 mg).

LCMS: (M+H)⁺=306, Retention time: 1.24 min. CP Method E

Step 4: Intermediate 49 was prepared analogously to the General Procedure GP-1. To a mixture of 1-(4-fluorophenyl)-6-(methylsulfonyl)-1,2,3,4-tetrahydroisoquinoline (160 mg, 0.52 mmol) and TEA (158 mg, 1.56 mmol) dissolved in DMF (5 ml) was added (S)-quinuclidin-3-amine (66 mg, 0.52 mmol) and CDI (168 mg, 1.04 mmol), the reaction mixture was stirred at 60° C. for 16 hr. The reaction mixture was diluted with ice-water (20 mL), extracted with two 20 mL portions of ethyl acetate. The combined organic phases were washed with brine, dried and concentrated to obtain a crude product, which was purified by prep-HPLC to obtain 1-(4-fluorophenyl)-6-(methylsulfonyl)-N—((S)-quinuclidin-3-yl)-3,4-dihydroisoquinoline-2(1H)-carboxamide 49 (70 mg).

The diastereomers of product 49 were separated by chiral SFC eluting with EtOH containing 0.2% methanol ammonia over an EnantioPak® IG column (4.6*250 mm 5 μm) to give Compound 2040 (retention time=18.039 min) and Compound 2041 (retention time=23.573 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2040: LCMS: (M+H)⁺=458; purity=100% (214 nm); Retention time=1.292 min. CP Method A

¹H NMR (400 MHz, DMSO-d₆) δ 7.83 (s, 1H), 7.77-7.69 (m, 1H), 7.43 (d, J=8.2 Hz, 1H), 7.18-7.13 (m, 3H), 6.59 (s, 1H), 6.42 (d, J=5.9 Hz, 1H), 3.96-3.84 (m, 1H), 3.74-3.64 (br, s, 1H), 3.26-3.19 (m, 4H), 3.12-2.93 (m, 3H), 2.89-2.77 (m, 2H), 2.73-2.57 (m, 3H), 1.82-1.73 (m, 2H), 1.55 (d, J=6.6 Hz, 2H), 1.30 (s, 1H).

Chiral SFC: n-hexane (containing 0.1% DEA)/EtOH (containing 0.1% DEA) over an ENANTIOPAK® IG column (4.6*250 mm 5 μm), retention time=18.039 min).

Compound 2041: LCMS: (M+H)⁺=458; purity=98.2% (214 nm); Retention time=1.287 min. CP Method A

¹H NMR (400 MHz, DMSO-d₆) δ 7.83 (s, 1H), 7.77-7.71 (m, 1H), 7.43 (d, J=8.1 Hz, 1H), 7.16 (dd, J=7.1, 3.0 Hz, 4H), 6.58 (s, 1H), 6.39 (d, J=6.2 Hz, 1H), 3.90 (dd, J=11.7, 6.3 Hz, 1H), 3.7-3.63 (br, s, 1H), 3.22 (s, 4H), 3.02 (ddd, J=19.8, 15.9, 7.4 Hz, 3H), 2.86 (d, J=16.6 Hz, 2H), 2.70-2.60 (m, 3H), 1.76 (d, J=2.9 Hz, 1H), 1.72-1.64 (br, s, 1H), 1.58-1.48 (m, 2H), 1.37-1.32 (m, 1H).

Chiral SFC: CO₂/MeOH containing 0.2% methanolic ammonia over an ENANTIOPAK® AS column (4.6*100 mm 5 μm), retention time=23.573 min).

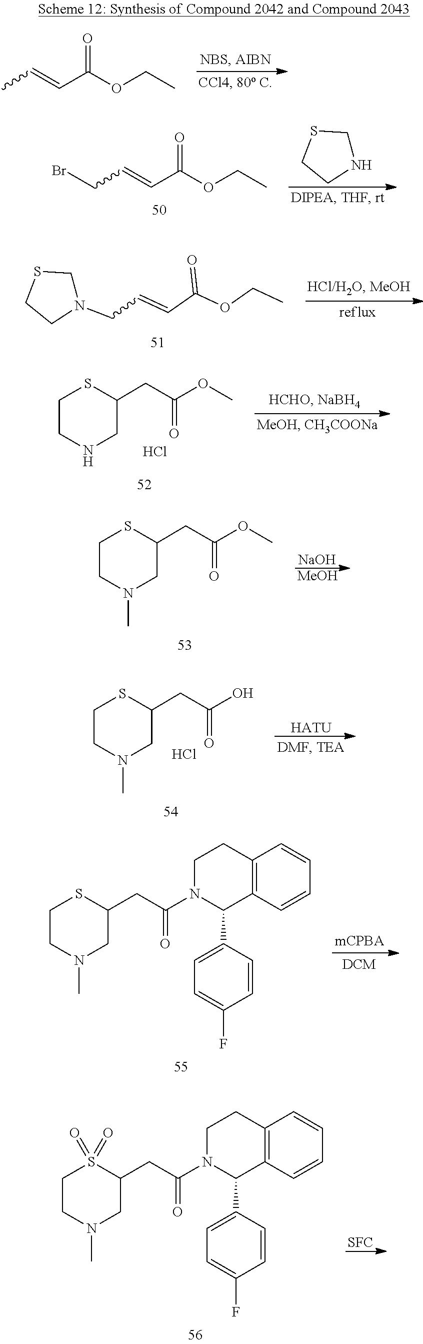

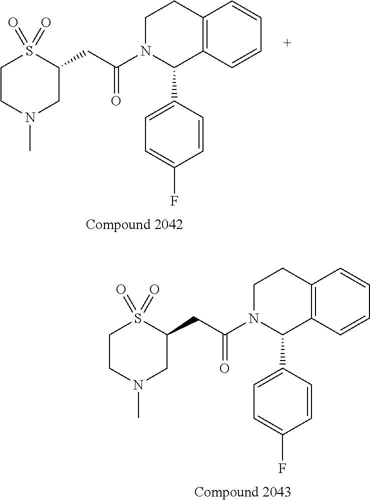 [SMILES: CN1CCS(=O)(=O)[C@H](CC(=O)N2CCc3ccccc3[C@@H]2c2ccc(F)cc2)C1 ; CN1CCS(=O)(=O)[C@@H](CC(=O)N2CCc3ccccc3[C@@H]2c2ccc(F)cc2)C1]

Step 1: To a solution of ethyl but-2-enoate (5 g, 43.8 mmol) in CCl₄ (75 mL) was added NBS (9.35 g, 52.6 mmol) at rt. After stirring for 5 min at the temperature, AIBN (0.36 g, 2.2 mmol) was added and the reaction mixture was heated to 80° C. for 16 h. After cooling, the reaction mixture was filtered. The filtrate was diluted by DCM (50 mL) and washed with water and brine. The organic layers were dried and concentrated. The crude compound was distilled to give the ethyl 4-bromobut-2-enoate 50 (5.24 g, 27.1 mmol) as a colorless oil.

LCMS: (M+1)⁺=192; Retention time=1.73 min. CP Method B

Step 2: To a solution of thiazolidine (5.6 g, 62.7 mmol) and DIPEA (18.8 mL, 114 mmol) in THF (120 mL) was added ethyl 4-bromobut-2-enoate 50 (11 g, 57 mmol) at rt. The reaction mixture was stirred at rt for 16 h. The reaction mixture was filtered and the filtrate was diluted with EtOAc (100 mL). The solution was washed with water, then with brine, dried and concentrated. The crude compound was purified by silica gel column chromatography (PE/EtOAc: 10:1 to 5:1) to give ethyl 4-(thiazolidin-3-yl)but-2-enoate 51 (10.1 g, 50 mmol) as a light yellow oil.

LCMS: (M+1)⁺=202; Retention time=1.41 min. CP Method C1

Step 3: The ethyl 4-(thiazolidin-3-yl)but-2-enoate 51 (2.5 g, 12.4 mmol) was suspended in 6 M HCl (20 mL) and MeOH (2 mL). The mixture was heated to 100° C. for 24 h. After cooling, the reaction was concentrated to give crude methyl 2-(thiomorpholin-2-yl)acetate hydrochloride 52 (2.22 g, 10.5 mmol) as light brown oil.

LCMS: (M+1)⁺=176; Retention time=0.62 min. CP Method B

Step 4: To a solution of methyl 2-(thiomorpholin-2-yl)acetate hydrochloride 52 (2.2 g, 10.4 mmol) in MeOH (30 mL) was added sodium acetate (0.94 g, 11.4 mmol) and 37% CH₂O (1.01 g, 12.5 mmol) at rt. The reaction was stirred at rt for 16 h. Then the NaBH₄ (0.79 g, 20.8 mmol) was added slowly. The reaction was stirred at rt for 3 h. The mixture was concentrated and the residue was dissolved in water and extracted with four 30 mL portions of DCM/MeOH (10:1). The combined organic layers were dried over Na₂SO₄, filtered and concentrated. The crude was purified by silica gel column (PE/EtOAc: 1:2) to give crude methyl 2-(4-methylthiomorpholin-2-yl)acetate 53 (380 mg, 2.2 mmol) as a light yellow oil.

LCMS: (M+1)⁺=190; Retention time=1.702 min. CP Method C1

Step 5: To a solution of methyl 2-(4-methylthiomorpholin-2-yl)acetate 53 (340 mg, 1.8 mmol) in MeOH (5 mL) was added 2 N NaOH (2.7 mL, 5.4 mmol) at rt. The reaction mixture was stirred at rt for 4 h The reaction was acidified by 6 N HCl to adjust to pH 3, and then concentrated. The residue was suspended twice in 20 mL MeOH/DCM (1:5, 20) and then filtered. The combined filtrate was concentrated to give crude 2-(4-methylthiomorpholin-2-yl)acetic acid hydrochloride 54 (410 mg) used for next step without further purification.

LCMS: (M+1)⁺=176; Retention time=0.33 min. CP Method C1

Step 6: To a solution of 2-(4-methylthiomorpholin-2-yl)acetic acid hydrochloride 54 (380 mg, 1.79 mmol) and TEA (0.75 mL, 0.27 mmol) in DMF (6 mL) was added HATU (680 mg, 1.79 mmol). The reaction mixture was stirred at rt for 10 h, then (S)-1-(4-fluorophenyl)-1,2,3,4-tetrahydroisoquinoline (407 mg, 1.79 mmol) was added and the reaction was stirred at rt for 16 h. The reaction mixture was purified by prep-HPLC to give 1-((S)-1-(4-fluorophenyl)-3,4-dihydroisoquinolin-2(1H)-yl)-2-(4-methylthiomorpholin-2-yl)ethanone 55 (263 mg, 0.68 mmol) as a white solid.

LCMS: (M+1)⁺=385; Retention time=1.846 min. CP Method C

Step 7: To a solution of 1-((S)-1-(4-fluorophenyl)-3,4-dihydroisoquinolin-2(1H)-yl)-2-(4-methylthiomorpholin-2-yl)ethanone 55 (250 mg, 0.65 mmol) in DCM (2 mL) was added mCPBA (230 mg, 1.33 mmol). The reaction mixture was stirred at rt for 16 h. The reaction was concentrated and the residue was purified by prep-HPLC to give compound 56 (173 mg, 0.41 mmol) as a white solid.

LCMS: (M+1)⁺=417; Retention time=1.423 min. CP Method B

Step 8: The compound 56 (173 mg, 0.41 mmol) was separated by SFC to give Compound 2042 (0.1 mmol) and Compound 2043 (0.08 mmol).

Chiral SFC condition: Instrument: SFC-80 (Thar, Waters); Column: OD 20*250 mm, 10 um (Daicel); Column temperature: 35° C.; Mobile phase: CO₂/MEOH (0.2% methanol ammonia)=75/25; Flow rate: 80 g/min; Back pressure: 100 bar; Detection wavelength: 214 nm; Cycle time: 4.5 min.

Compound 2042: LCMS: (M+1)⁺=417; Retention time=1.404 min. CP Method C

¹HNMR (400 Hz, CD₃OD): δ 7.23-7.16 (m, 5H), 7.09-6.96 (m, 3H), 6.79 (s, 1H), 3.99-3.92 (m, 2H), 3.81-3.72 (m, 2H), 3.56-3.41 (m, 2H), 3.31-3.24 (m, 5H), 3.11-3.04 (m, 1H), 2.95-2.85 (m, 2H), 2.79-2.73 (m, 1H), 2.59-2.53 (m, 1H).

Chiral SFC: MeOH (0.2% methanol ammonia) over an ENANTIOPAK® IG column (4.6*250 mm 5 μm), retention time=6.89 min), 100% ee.

Compound 2043:

LCMS: (M+1)⁺=417; Retention time=1.402 min. CP Method C

¹HNMR (400 Hz, CD₃OD): δ 7.23-7.16 (m, 5H), 7.07-6.95 (m, 3H), 6.77 (s, 1H), 4.10-3.98 (m, 2H), 3.89-3.81 (m, 2H), 3.62-3.46 (m, 2H), 3.36-3.31 (m, 4H), 3.17-2.81 (m, 4H), 2.68-2.63 (m, 1H).

Chiral SFC: MeOH (0.2% methanol ammonia) over an ENANTIOPAK® IG column (4.6*250 mm 5 μm), retention time=12.277 min), 98% ee.

The following compounds were prepared using General Procedure GP-4.

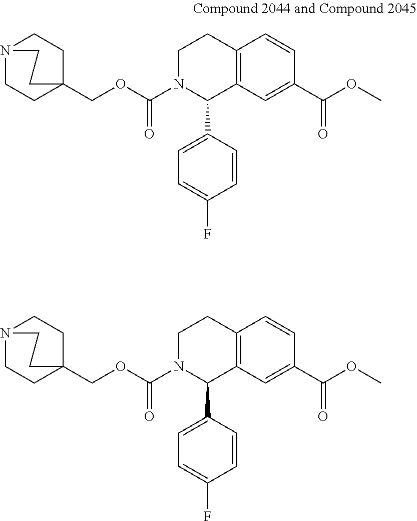 [SMILES: COC(=O)c1ccc2c(c1)[C@H](c1ccc(F)cc1)N(C(=O)OCC13CCN(CC1)CC3)CC2 ; COC(=O)c1ccc2c(c1)[C@@H](c1ccc(F)cc1)N(C(=O)OCC13CCN(CC1)CC3)CC2]

The diastereomers were separated by chiral SFC eluting with CO₂/EtOH containing 1% methanolic ammonia over an EnantioPak® AD column (20*250 mm 10 μm) to give Compound 2044 (retention time=1.79 min) and Compound 2045 (retention time=2.53 min). Stereochemical assignment at 1 position of the tetrahydroisoquinoline is assigned based on chromatographic elution order as compared to diastereomers of related analogues of known configuration.

Compound 2044: LCMS: (M+H)⁺=453; (214 nm); retention time=1.521 min. CP Method C1

¹H NMR (400 MHz, CD₃OD) δ 7.90 (dd, J=8.0, 1.6 Hz, 1H), 7.77 (s, 1H), 7.39 (d, J=8.0 Hz, 1H), 7.25 (s, 2H), 7.07 (t, J=8.4 Hz, 2H), 6.40 (s, 1H), 4.04 (dt, J=13.2, 4.8 Hz, 1H), 3.08-2.99 (m, 1H), 2.98-2.88 (m, 7H), 1.52 (s, 6H).

Chiral SFC: MeOH (0.2% methanol ammonia) over an ENANTIOPAK® IG column (4.6*100 mm 5 μm), retention time=4.02 min), 100% ee.

Compound 2045: LCMS: (M+H)⁺=453; (214 nm); retention time=1.467 min. CP Method A1

¹H NMR (400 MHz, CD₃OD) δ 7.90 (dd, J=8.0, 1.2 Hz, 1H), 7.77 (s, 1H), 7.39 (d, J=8.0 Hz, 1H), 7.25 (s, 2H), 7.07 (t, J=8.4 Hz, 2H), 6.39 (s, 1H), 4.04 (dt, J=13.2, 4.8 Hz, 1H), 3.09-2.88 (m, 8H), 1.52 (s, 6H).

Chiral SFC: MeOH (0.2% methanol ammonia) over an ENANTIOPAK® IG column (4.6*100 mm 5 μm), retention time=5.47 min), 99% ee.

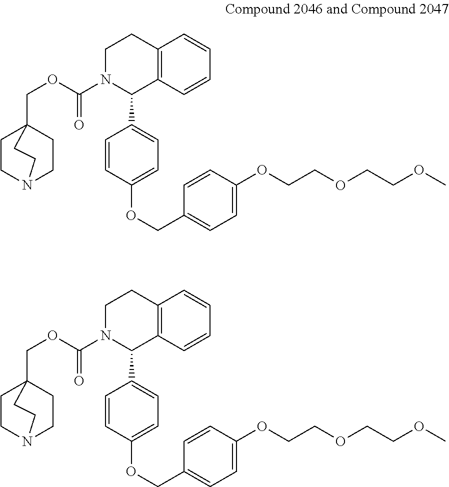 [SMILES: COCCOCCOc1ccc(COc2ccc([C@H]3c4ccccc4CCN3C(=O)OCC34CCN(CC3)CC4)cc2)cc1 ; COCCOCCOc1ccc(COc2ccc([C@H]3c4ccccc4CCN3C(=O)OCC34CCN(CC3)CC4)cc2)cc1]

The diastereomers were separated by chiral SFC eluting with CO₂/EtOH containing 0.5 methanol ammonia over an EnantioPak® OJ column (20*250 mm 10 μm) to give Compound 2046 (retention time=10.257 min) and Compound 2047 (retention time=15.024 min). Stereochemical assignment at 1 position of the tetrahydroisoquinoline is assigned based on chromatographic elution order as compared to diastereomers of related analogues of known configuration.

Compound 2046: LCMS: (M+H)⁺=601; (214 nm); retention time=1.550 min. CP Method A1

¹H NMR (400 MHz, CD₃OD) δ 7.34 (d, J=8.4 Hz, 2H), 7.25-7.16 (m, 3H), 7.16-7.04 (br, 3H), 6.98-6.89 (m, 4H), 6.27 (brs, 1H), 4.98 (s, 2H), 4.16-4.12 (m, 2H), 4.02-3.87 (m, 3H), 3.86-3.82 (m, 2H), 3.72-3.68 (m, 2H), 3.60-3.56 (m, 2H), 3.38 (s, 4H), 3.09-3.00 (br, 6H), 3.00-2.90 (m, 1H), 2.88-2.79 (m, 1H), 1.60 (s, 6H).

Chiral SFC: MeOH (0.2% methanol ammonia) over an ENANTIOPAK® IG column (4.6*250 mm 5 μm), retention time=11.478 min).

Compound 2047: LCMS: (M+H)⁺=601; purity=100% (214 nm); retention time=1.551 min. CP Method A1

¹H NMR (400 MHz, CD₃OD) δ 7.34 (d, J=8.8 Hz, 2H), 7.25-7.22 (m, 2H), 7.22-7.17 (m, 1H), 7.16-7.06 (br, 3H), 6.98-6.89 (m, 4H), 6.27 (brs, 1H), 4.98 (s, 2H), 4.13 (t, J=4.4 Hz, 2H), 4.02-3.90 (m, 3H), 3.84 (t, J=4.4 Hz, 2H), 3.72-3.68 (m, 2H), 3.60-3.56 (m, 2H), 3.38 (s, 4H), 3.09 (brs, 6H), 3.02-2.91 (m, 1H), 2.89-2.79 (m, 1H), 1.64 (s, 6H).

Chiral SFC: MeOH (0.2% methanol ammonia) over an ENANTIOPAK® IG column (4.6*250 mm 5 μm), retention time=14.779 min).

The following compounds were prepared using procedure GP-1:

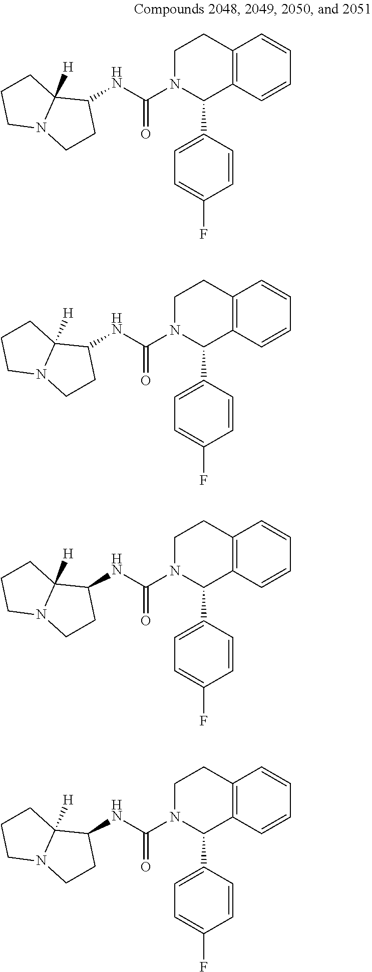

The diastereomers were separated by chiral SFC eluting with CO₂/EtOH containing 0.5 methanol ammonia over an EnantioPak® OJ column (20*250 mm 10 μm) to give Compound 2048 (retention time=5.745 min) Compound 2049 (retention time=7.682 min), Compound 2050 0 (retention time=18.212 min), and Compound 2051 (retention time=24.619 min). Stereochemical assignment of (S) at 1 position of the tetrahydroisoquinoline is based on the use of chiral THIQ while the stereochemical assignments at the pyrrolizidine centers are assigned randomly based on chromatographic elution order.

Compound 2048: LCMS: (M+H)⁺=380.2; (214 nm); retention time=1.442 min. CP Method C

¹H NMR (400 MHz, CD₃OD) δ7.27-7.15 (m, 6H), 7.05-7.00 (m, 2H), 6.4 (s, 1H), 4.39-4.37 (m, 1H), 3.81-3.78 (m, 2H), 3.46-3.31 (m, 1H), 3.16-3.12 (m, 1H), 3.04-2.97 (m, 1H), 2.87-2.75 (m, 2H), 2.81-2.67 (m, 2H), 2.07-2.01 (m, 2H), 1.81-1.75 (m, 1H), 1.72-1.70 (m, 2H), 1.52-1.45 (m, 1H).

Chiral SFC: n-hexane (0.1% DEA):EtOH (0.1% DEA)=30:70 over an ENANTIOPAK® IG column (4.6*250 mm 5 μm), retention time=5.745 min), 100% ee.

Compound 2049: LCMS: (M+H)⁺=380.2; (214 nm); retention time=1.431 min. CP Method C

¹H NMR (400 MHz, CD₃OD) δ8.56 (s, 1H), 7.28-7.15 (m, 6H), 7.19-7.00 (m, 2H), 6.48 (s, 1H), 4.44-4.35 (m, 2H), 3.79-3.50 (m, 2H), 3.48-3.33 (m, 2H), 3.07-2.94 (m, 3H), 2.82-2.77 (m, 1H), 2.28-2.09 (m, 3H), 1.95-1.90 (m, 3H)

Chiral SFC: n-hexane (0.1% DEA):EtOH (0.1% DEA)=30:70 over an ENANTIOPAK® IG column (4.6*250 mm 5 μm), retention time=7.682 min), 100% ee.

Compound 2050: LCMS: (M+H)⁺=380.2; (214 nm); retention time=1.463 min. CP Method C

¹H NMR (400 MHz, CD₃OD) δ 7.27-7.17 (m, 6H), 7.03-6.99 (t, J=8.4 Hz, 2H), 6.46 (s, 1H), 4.39-4.37 (m, 1H), 3.91-3.86 (dd, J=14.4 Hz, J=6.8 Hz, 2H), 3.66-3.59 (m, 1H), 3.33-3.26 (m, 2H), 3.15-3.11 (m, 1H), 2.90-2.80 (m, 2H), 2.68-2.57 (m, 3H), 2.13-2.08 (m, 1H), 1.93-1.90 (m, 2H), 1.83-1.65 (m, 3H).

Chiral SFC: n-hexane (0.1% DEA):EtOH (0.1% DEA)=30:70 over an ENANTIOPAK® IG column (4.6*250 mm 5 μm), retention time=18.212 min), 100% ee.

Compound 2051: LCMS: (M+H)⁺=380.2; (214 nm); retention time=1.458 min. CP Method C

¹H NMR (400 MHz, CD₃OD) δ 7.27-7.15 (m, 6H), 6.90 (t, J=8.8 Hz, 2H), 6.4 (s, 1H), 3.91-3.86 (dd, J=14.4 Hz, J=6.4 Hz 1H), 3.67-3.61 (m, 1H), 3.33-3.27 (m, 1H), 3.22-3.20 (m, 2H), 2.89-2.80 (m, 2H), 2.68-2.58 (m, 3H), 2.14-2.10 (m, 1H), 1.90-1.63 (m, 5H)

Chiral SFC: n-hexane (0.1% DEA):EtOH (0.1% DEA)=30:70 over an ENANTIOPAK® IG column (4.6*250 mm 5 μm), retention time=24.619 min), 100% ee.

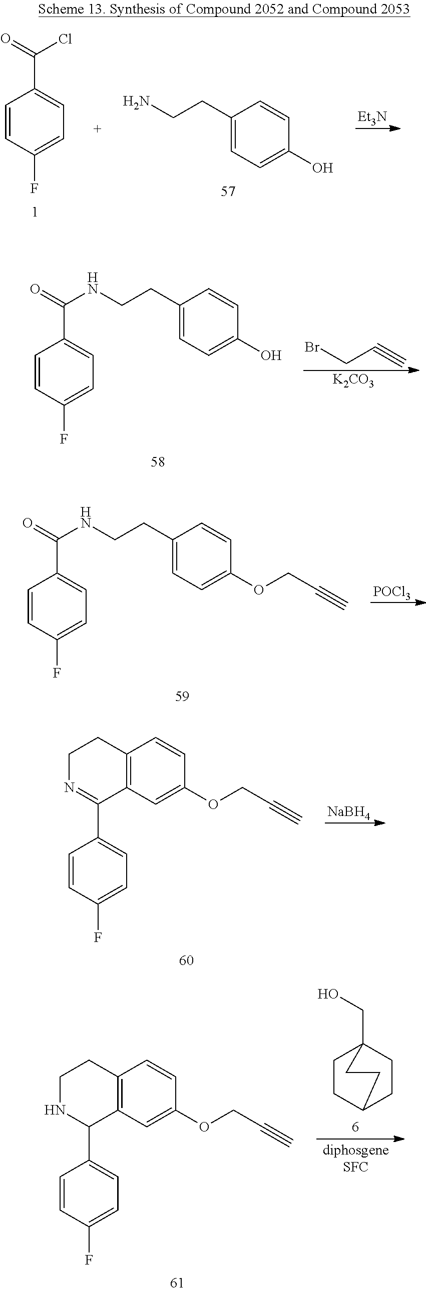

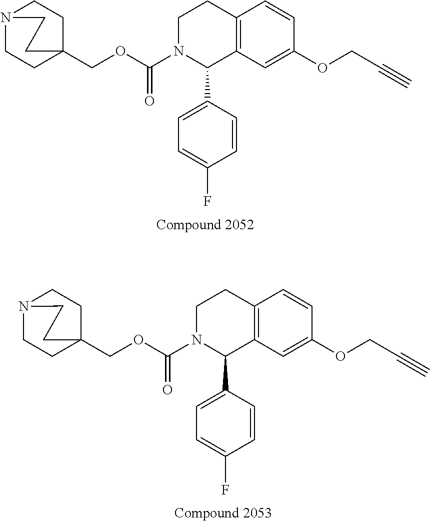 [SMILES: C#CCOc1ccc2c(c1)[C@H](c1ccc(F)cc1)N(C(=O)OCC13CCN(CC1)CC3)CC2 ; C#CCOc1ccc2c(c1)[C@@H](c1ccc(F)cc1)N(C(=O)OCC13CCN(CC1)CC3)CC2]

Step 1: To a solution of 4-(2-aminoethyl)phenol 57 (164 mg. 1.2 mmol) in 4 mL DCM and Et₃N (150 mg, 1.5 mmol) was added, then 4-fluorobenzoyl chloride 1 (158 mg, 1 mmol) in 1 mL DCM slowly. After the addition, the mixture was stirred at room temperature for 2 hrs. Water was added to quench the reaction and it was extracted with two 10 mL portions of DCM. The combined the organic phases were dried, filtered, and concentrated to give the 4-fluoro-N-(4-hydroxyphenethyl)benzamide 58 (250 mg) used directly without further purification.

LCMS: (M+H)⁺=260 (214 nm); retention time=1.388 min. CP Method A

Step 2: To the solution of 4-fluoro-N-(4-hydroxyphenethyl)benzamide 58 (250 mg. 0.96 mmol) in 4 mL of DMF was added K₂CO₃(265 mg, 1.92 mmol). Then 3-bromoprop-1-yne (172 mg, 1.45 mmol) in 1 mL DMF was slowly added. After the addition was complete, the mixture was stirred at room temperature overnight. Water was added to quench the reaction and it was extracted with two 10 mL portions of DCM. The combined organic phases were dried, filtered, and concentrated. The resulting residue was purified by silica gel column (PE:EA=3:1) to give the 4-fluoro-N-(4-(prop-2-ynyloxy)phenethyl)benzamide 59 (240 mg) as a solid.

LCMS: (M+H)⁺=298 (214 nm); retention time=1.728 min. CP Method A

Step 3: In a 100 mL round bottom flask, 4-fluoro-N-(4-(prop-2-ynyloxy)phenethyl)benzamide 59 (200 mg, 0.67 mmol) was dissolved in 5 ml POCl₃, then P₂O₅(190 mg, 1.34 mmol) was added. The mixture was heated under reflux for four hours The reaction was cooled using an ice-bath, then alkalized to pH 10 using NaOH (2N). The mixture was extracted with three 20 mL portions of DCM, and the combined organic phases were dried. The material was concentrated to give 150 mg of 1-(4-fluorophenyl)-7-(prop-2-ynyloxy)-3,4-dihydroisoquinoline 60 as a yellow solid.

LCMS: (M+H)⁺=280 (214 nm); retention time=1.343 min. CP Method A

Step 4: To a solution of 1-(4-fluorophenyl)-7-(prop-2-ynyloxy)-3,4-dihydroisoquinoline 60 (150 mg, 0.54 mmol) in 5 mL MeOH was added NaBH₄ (61 mg, 1.62 mmol). The mixture was stirred at room temperature for two hours, The solvent was removed in vacuo and the mixture was diluted with 10 mL of DCM. The mixture was washed with saturated NaHCO₃ solution and 15 mL of water. The organic phase was dried and concentrated to give 150 mg of 1-(4-fluorophenyl)-7-(prop-2-ynyloxy)-1,2,3,4-tetrahydroisoquinoline 61 which was used without further purification.

LCMS: (M+H)⁺=282 (214 nm); retention time=1.361 min. CP Method A

Step 5: To a solution of 61 (141 mg, 1 mmol) in 3 mL of CH₃CN, diphosgene (218 mg, 1.1 mmol) was added, the mixture was stirred at room temperature for one hour. The solution became clear and the solvent was removed to give the white solid for the next step. The white solid was dissolved in 5 mL of DMF, alcohol 6 (225 mg, 0.8 mmol) and Et₃N (303 mg, 3 mmol) were added. The mixture was heated to 80° C. overnight. 10 mL of water was added and the mixture extracted with three 10 mL portions of DCM. The combined organic phases were dried, filtered, and concentrated in vacuo to give a residue which was purified by prep-HPLC and SFC to give the (S)-quinuclidin-4-ylmethyl 1-(4-fluorophenyl)-7-(prop-2-ynyloxy)-3,4-dihydroisoquinoline-2(1H)-carboxylate as a white solid.

The diastereomers were separated by chiral SFC eluting with n-hexane containing 0.1% DEA/EtOH containing 0.1% DEA over an EnantioPak® AY column (20*250 mm 10 μm) to give Compound 2052 (retention time=7.751 min) and Compound 2053 (retention time=9.033 min). Stereochemical assignment at 1 position of the tetrahydroisoquinoline is assigned based on chromatographic elution order as compared to diastereomers of related analogues of known configuration.

Compound 2052: LCMS: (M+H)⁺=449; retention time=1.648 min. CP Method C

¹H NMR (400 MHz, CDCl₃) δ 7.22-7.19 (m, 2H), 7.14 (d, J=8.0 Hz, 1H), 7.00-6.97 (m, 2H), 6.89 (d, J=7.8 Hz, 1H), 6.66 (s, 1H), 6.40 (brs, 1H), 4.63 (s, 2H), 4.09-3.86 (m, 3H), 3.37-3.20 (m, 2H), 3.02-2.95 (m, 6H), 2.75-2.71 (m, 1H), 2.48 (s, 1H), 1.63-1.60 (m, 6H).

Chiral SFC: n-hexane (0.1% DEA):EtOH (0.1% DEA)=40:60 over an ENANTIOPAK® IG column (4.6*250 mm 5 μm), retention time=12.733 min), 100% ee.

Compound 2053: LCMS: (M+H)⁺=449; retention time=1.670 min. CP Method C

¹H NMR (400 MHz, CDCl₃) δ 7.21-7.18 (m, 2H), 7.13 (d, J=8.4 Hz, 1H), 6.99-6.95 (m, 2H), 6.88 (d, J=8.4 Hz, 1H), 6.65 (s, 1H), 6.39 (brs, 1H), 4.63 (s, 2H), 4.09-3.82 (m, 3H), 3.25-3.21 (m, 1H), 2.96-2.91 (m, 7H), 2.75-2.69 (m, 1H), 2.47 (s, 1H), 1.49-1.37 (m, 6H).

Chiral SFC: n-hexane (0.1% DEA):EtOH (0.1% DEA)=40:60 over an ENANTIOPAK® IG column (4.6*250 mm 5 μm), retention time=16.955 min), 100% ee.

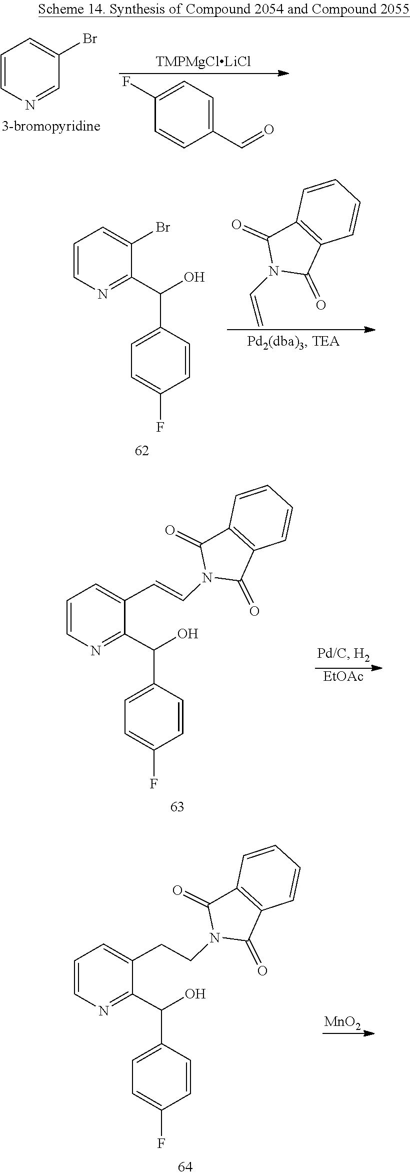

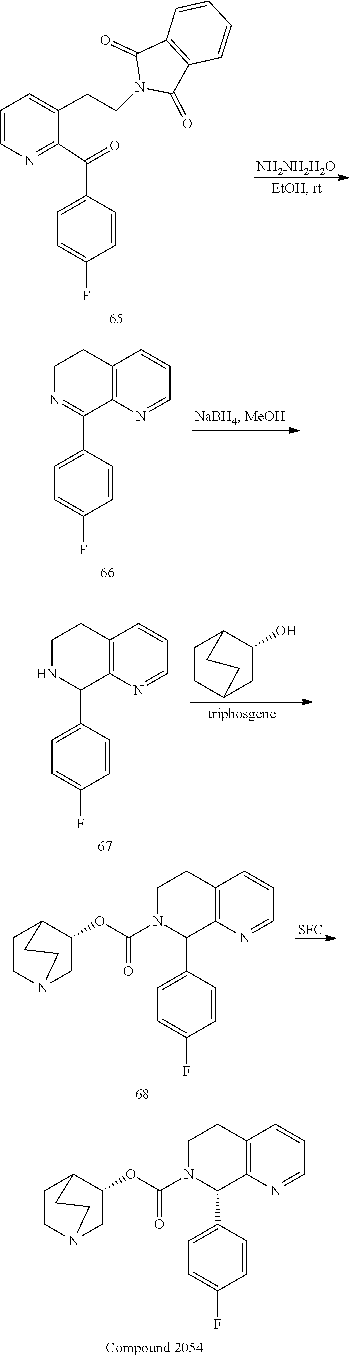

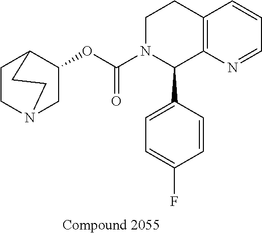 [SMILES: O=C(O[C@@H]1CN2CCC1CC2)N1CCc2cccnc2[C@H]1c1ccc(F)cc1]

Step 1: To a solution of 3-bromopyridine (30 g, 190 mmol) in dry THF (300 mL) was added dropwise TMPMgCl.LiCl (228 mL, 228 mmol) at 0° C. After stirring for 30 min at that temperature, a solution of 4-fluorobenzaldehyde (26 g, 209 mmol) in THF (5 mL) was added dropwise to the reaction. Then the reaction was stirred at rt for 16 h. The reaction mixture was poured into ice-water (300 mL) and extracted with EtOAc (150 mL×3). The combined organic layers were dried and concentrated. The crude compound was purified by silica gel column chromatography (PE/EtOAc, 20:1 to 10:1) to give (3-bromopyridin-2-yl)(4-fluorophenyl)methanol 62 (13.79 g, 48.9 mmol) as an orange solid.

LCMS: (M+1)⁺=283; Retention time=1.53 min. CP Method D

Step 2: To a solution of (3-bromopyridin-2-yl)(4-fluorophenyl)methanol 62 (13.79 g, 48.9 mmol), 2-vinylisoindoline-1,3-dione (9.31 g, 53.8 mmol), TEA (13.6 mL, 98 mmol), CyJohnPhos (1.71 g, 4.9 mmol) in DMF (250 mL) was added Pd₂(dba)₃ (2.24 g, 2.4 mmol). The reaction was evacuated and refilled with N₂. Then the reaction was heated 100° C. for 16 h. After cooling, the reaction was filtered and the filtrated was concentrated. The residue was suspended in EtOAc/PE (2:1, 50 mL) and stirred for 5 min. The solid was collected by filtration and dried to give (E)-2-(2-(2-((4-fluorophenyl)(hydroxy)methyl)pyridin-3-yl)vinyl)isoindoline-1,3-dione 63 (11 g, 29 mmol) as a brown solid.

LCMS: (M+1)⁺=375; Retention time=1.478 min. CP Method A2

Step 3: To a suspension of (E)-2-(2-(2-((4-fluorophenyl)(hydroxy)methyl)pyridin-3-yl)vinyl)isoindoline-1,3-dione 63 (3.45 g, 9.7 mmol) in THF (100 mL) was added 10% Pd/C (0.8 g, 0.7 mmol). The reaction mixture was evacuated and then refilled with hydrogen. The reaction was stirred for 16 h at rt. The reaction mixture was filtered and concentrated to give 2-(2-(2-((4-fluorophenyl)(hydroxy)methyl)pyridin-3-yl)ethyl)isoindoline-1,3-dione 64 (1.99 g, 4.4 mmol) as a light yellow oil.

LCMS: (M+1)⁺=377; Retention time=1.543 min. CP Method D

Step 4: To a solution of 2-(2-(2-((4-fluorophenyl)(hydroxy)methyl)pyridin-3-yl)ethyl)isoindoline-1,3-dione 64 (1.99 g, 5.3 mmol) in DCM (50 mL) was added MnO₂ (4.6 g, 53 mmol). The reaction mixture was heated to 38° C. and stirred for 2 days. The reaction mixture was filtered and the filtrate was concentrated to give 2-(2-(2-(4-fluorobenzoyl)pyridin-3-yl)ethyl)isoindoline-1,3-dione 65 (1.62 g, 4.4 mmol) as a light yellow oil.

LCMS: (M+1)⁺=375; Retention time=1.772 min. CP Method A2

Step 5: To a suspension of 2-(2-(2-(4-fluorobenzoyl)pyridin-3-yl)ethyl)isoindoline-1,3-dione 65 (1.62 g, 9.7 mmol) in EtOH (50 mL) was added 85% H₂NNH₂.H₂O (0.76 g, 13 mmol) at rt. The reaction mixture was stirred for 16 h at rt. The reaction mixture was filtered and concentrated to give crude 8-(4-fluorophenyl)-5,6-dihydro-1,7-naphthyridine 66 (1.4 g, 6.2 mmol) as a light brown oil.

LCMS: (M+1)⁺=227; Retention time=1.536 min. CP Method C

Step 6: The crude compound 8-(4-fluorophenyl)-5,6-dihydro-1,7-naphthyridine 66 (1.4 g, 6.2 mmol) was dissolved in MeOH (30 mL). Then NaBH₄ (0.47 g, 12.3 mmol) was added slowly. The reaction was stirred at rt for 16 h. The reaction was concentrated and the residue was dissolved in water (30 mL). The mixture as extracted with EtOAc (50 mL×3). The combined organic layers were dried and concentrated. The residue was purified by prep-HPLC to give 8-(4-fluorophenyl)-5,6,7,8-tetrahydro-1,7-naphthyridine 67 (566 mg, 2.48 mmol) as a light brown oil.

LCMS: (M+1)⁺=229; Retention time=1.374 min. CP Method C

Step 7: To a suspension of (S)-quinuclidin-3-ol (200 mg, 1.58 mmol) in dry CH₃CN (10 mL) was added triphosgene (232 mg, 0.79 mmol) at rt. The reaction mixture was heated to 25° C. for 2 h. The reaction was concentrated and the residue was dissolved in dry DMF (10 mL). Then TEA (0.33 mL, 2.37 mmol) and 8-(4-fluorophenyl)-5,6,7,8-tetrahydro-1,7-naphthyridine 67 (180 mg, 0.79 mmol) was added to the reaction. The reaction was heated to 80° C. for 3 h. The reaction was purified by prep-HPLC to give (S)-quinuclidin-3-yl 8-(4-fluorophenyl)-5,6-dihydro-1,7-naphthyridine-7(8H)-carboxylate 68 (20 mg, 0.05 mmol) as a brown oil.

LCMS: (M+1)⁺=382; Retention time=1.63 min. CP Method D

Step 8: The (S)-quinuclidin-3-yl 8-(4-fluorophenyl)-5,6-dihydro-1,7-naphthyridine-7(8H)-carboxylate 68 (28 mg, 0.07 mmol) was separated by SFC to give Compound 2054 (0.02 mmol) and Compound 2055 (0.004 mmol).

Chiral SFC condition: Instrument: SFC-80 (Thar, Waters); Column: IG 20*250 mm, 10 um (Daicel); Column temperature: 35° C.; Mobile phase: CO₂/MeOH (0.2% methanol ammonia)=40/60; Flow rate: 80 g/min; Back pressure: 100 bar; Detection wavelength: 214 nm; Cycle time: 10 min; Sample solution: 28 mg dissolved in 8 ml methanol; Injection volume:4.5 mL.

Compound 2054: LCMS: (M+1)⁺=382; Retention time=1.325 min. CP Method A2

¹H NMR (400 MHz, CDCl₃) δ 8.40 (d, J=3.6 Hz, 1H), 7.76 (d, J=7.6 Hz, 1H), 7.36 (dd, J=4.8, 7.6 Hz, 1H), 7.23 (br, 2H), 7.07 (t, J=8.8 Hz, 2H), 6.42 (br, 1H), 4.87-4.83 (m, 1H), 4.13-4.10 (m, 1H), 3.32-3.24 (m, 1H), 3.11-3.03 (m, 1H), 2.95-2.73 (m, 6H), 2.11 (d, 2.8 Hz, 1H), 1.86-1.76 (m, 2H), 1.71-1.62 (m, 1H), 1.57-1.50 (m, 1H).

Chiral SFC: MeOH (0.2% methanol ammonia) over an ENANTIOPAK® IG column (4.6*100 mm 5 μm), retention time=2.41 min).

Compound 2055: LCMS: (M+1)⁺=382; Retention time=1.391 min. CP Method E

¹H NMR (400 MHz, CDCl₃) δ 8.41 (d, J=4 Hz, 1H), 7.77 (d, J=6.8 Hz, 1H), 7.38-7.08 (m, 5H), 6.45-6.35 (m, 1H), 4.89-4.84 (m, 1H), 4.14-4.02 (m, 1H), 6.62-3.49 (m, 1H), 3.29-3.24 (m, 1H), 2.95-2.71 (m, 7H), 2.09 (br, 1H), 1.91-1.76 (m, 2H), 1.66-1.62 (m, 1H), 1.55-1.50 (m, 1H).

Chiral SFC: MeOH (0.2% methanol ammonia) over an ENANTIOPAK® IG column (4.6*100 mm 5 μm), retention time=3.51 min).

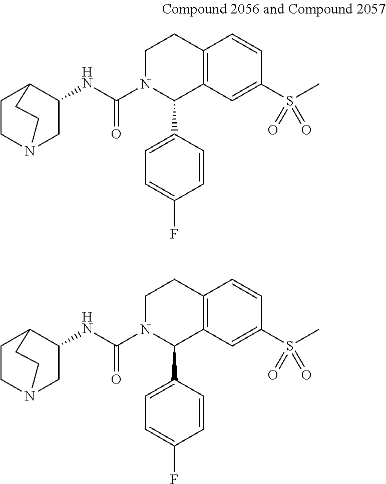

The diastereomers were separated by chiral SFC eluting with EtOH containing 0.2% methanol ammonia over an EnantioPak® IG column (4.6*100 mm 5 μm) to give Compound 2056 (retention time=2.4 min) and Compound 2057 (retention time=13.29 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2056: LCMS: (M+H)⁺=458 Retention time=1.338 min. CP Method C

¹H NMR (400 MHz, DMSO-d₆) δ 7.79 (dd, J=8.0, 1.9 Hz, 1H), 7.72-7.68 (brs, 1H), 7.54 (d, J=8.1 Hz, 1H), 7.20-7.13 (m, 4H), 6.62 (s, 1H), 6.40 (d, J=6.1 Hz, 1H), 3.90 (dt, J=13.1, 4.8 Hz, 1H), 3.77-3.69 (m, 1H), 3.25-3.21 (m, 1H), 3.20 (s, 3H), 3.14-3.06 (m, 1H), 3.00-2.92 (m, 1H), 2.90-2.80 (m, 2H), 2.75-2.61 (m, 4H), 1.82-1.76 (m, 1H), 1.75-1.66 (m, 1H), 1.63-1.48 (m, 2H), 1.34-1.26 (m, 1H).

Chiral SFC: MeOH/CAN=1/1 (0.1% DEA) over an ENANTIOPAK® IG column (4.6*100 mm 5 μm), retention time=2.45 min), 100% ee.

Compound 2057:

LCMS: (M+H)⁺=458; purity=100% (214 nm); Retention time=1.353 min. CP Method C

¹H NMR (400 MHz, DMSO-d₆) δ 7.79 (dd, J=8.0, 1.8 Hz, 1H), 7.70 (s, 1H), 7.54 (d, J=8.1 Hz, 1H), 7.16 (d, J=7.2 Hz, 4H), 6.67-6.61 (brs, 1H), 6.38 (d, J=5.8 Hz, 1H), 3.90 (dt, J=12.9, 4.9 Hz, 1H), 3.73-3.61 (m, 1H), 3.26-3.22 (m, 1H), 3.19 (s, 3H), 3.09-2.92 (m, 2H), 2.89-2.75 (m, 2H), 2.72-2.58 (m, 3H), 2.57-2.53 (m, 1H), 1.82-1.69 (m, 2H), 1.62-1.45 (m, 2H), 1.33-1.25 (m, 1H).

Chiral SFC: MeOH/CAN=1/1 (0.1% DEA) over an ENANTIOPAK® IG column (4.6*100 mm 5 μm), retention time=13.25 min), 100% ee.

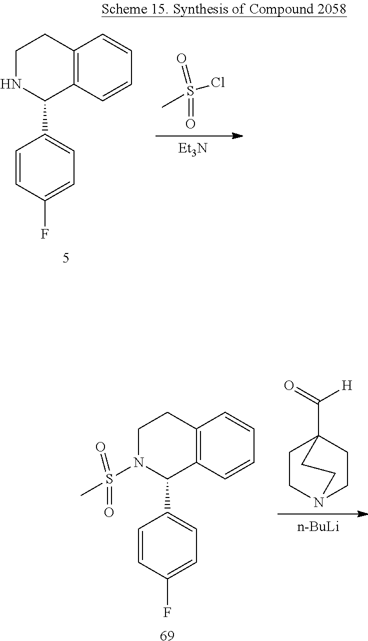

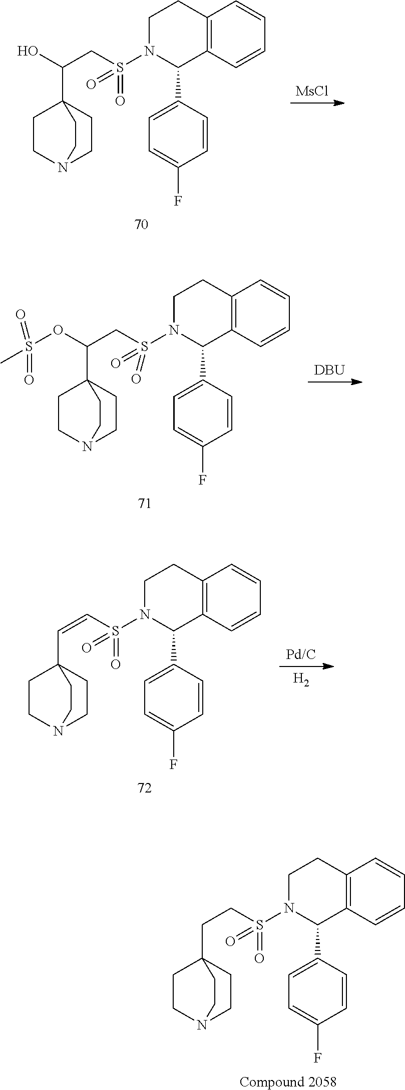

Step 1: To the solution of (S)-1-(4-fluorophenyl)-1,2,3,4-tetrahydroisoquinoline (454 mg. 2 mmol) in 4 mL of DCM, was added Et₃N (404 mg, 4 mmol), then methanesulfonyl chloride (342 mg, 1.5 mmol) in 1 mL DCM was slowly added. The mixture was stirred at room temperature for 2 hrs. Water was added to quench the reaction and the mixture was extracted with two 10 mL portions of DCM. The combined organic phases were dried, filtered, and concentrated to give the desired product 69 (550 mg) which was used without further purification in subsequent reactions.

LCMS: (M+H)⁺=305 (214 nm); retention time=1.373 min. CP Method A

Step 2: To a solution of (S)-1-(4-fluorophenyl)-2-(methylsulfonyl)-1,2,3,4-tetrahydroisoquinoline 69 (152 mg, 0.5 mmol) in 4 mL of dry THF cooled to −78° C. under the nitrogen, was slowly added n-BuLi (2.5M in hexane, 0.24 mL). The mixture was stirred at −78° C. for 10 minutes, quinuclidine-4-carbaldehyde (278 mg, 2 mmol) in 1 mL THF was added, and the mixture was allowed to react equilibrating to room temperature. After 10 hours, the reaction was diluted with 10 mL of EA and 5 mL of water was added. The mixture was extracted with three 10 mL portions of EA, and the combined organic layers were washed with 10 mL brine, dried, and concentrated. The residue was purified by prep-HPLC to give 110 mg of 2-((S)-1-(4-fluorophenyl)-3,4-dihydroisoquinolin-2(1H)-ylsulfonyl)-1-(quinuclidin-4-yl)ethanol 70 as a white solid.

LCMS: (M+H)⁺=445 (214 nm); retention time=1.483 min. CP Method A

Step 3: To the solution of 2-((S)-1-(4-fluorophenyl)-3,4-dihydroisoquinolin-2(1H)-ylsulfonyl)-1-(quinuclidin-4-yl)ethanol 70 (111 mg. 0.25 mmol) in 2 mL of DCM was added Et₃N (51 mg, 0.5 mmol). Then methanesulfonyl chloride (57 mg, 0.5 mmol) in 1 mL of DCM was slowly added. After addition, the mixture was stirred at room temperature for 2 hrs. Water was added to quench the reaction and the mixture was extracted with two 10 mL portions of DCM. The combined the organic phases were dried, filtered, and concentrated to give the desired product 71 (131 mg) which was used in subsequent reactions without further purification.

LCMS: (M+H)⁺=523 (214 nm); retention time=1.619 min. CP Method A

Step 4: To a solution of 2-((S)-1-(4-fluorophenyl)-3,4-dihydroisoquinolin-2(1H)-ylsulfonyl)-1-(quinuclidin-4-yl)ethyl methanesulfonate 71 (131 mg. 0.25 mmol) in 5 mL of THF was added DBU (76 mg, 0.5 mmol). The mixture was stirred at room temperature for 3 hours. The reaction was diluted with 10 mL of EA and 5 mL of water was added. The mixture was extracted with three 10 mL portions of EA the combined organic layers were washed with 10 mL of brine, dried, and concentrated to give 107 mg of (S,E)-4-(2-(1-(4-fluorophenyl)-3,4-dihydroisoquinolin-2(1H)-ylsulfonyl)vinyl)quinuclidine 72 as a white solid which was used in subsequent reactions without further purification.

LCMS: (M+H)⁺=427 (214 nm); retention time=1.585 min. CP Method A

Step 5: To a solution of (S,E)-4-(2-(1-(4-fluorophenyl)-3,4-dihydroisoquinolin-2(1H)-ylsulfonyl)vinyl)quinuclidine 72 (107 mg, 0.25 mmol) in 5 mL of anhydrous EtOH, was added 15 mg Pd/C and the reaction placed under an atmosphere of hydrogen. The mixture was heated to 50° C. When the reaction was complete, it was filtered and the filtrate concentrated in vacuo. The residue was purified by prep-HPLC to give the (S)-4-(2-(1-(4-fluorophenyl)-3,4-dihydroisoquinolin-2(1H)-ylsulfonyl)ethyl)quinuclidine, compound 2058.

LCMS: (M+H)⁺=429 (214 nm); retention time=1.578 min. CP Method A

Compound 2058: LCMS: (M+H)⁺=429; retention time=1.863 min. CP Method C

¹H NMR (400 MHz, CD₃OD) δ 7.31-7.27 (m, 5H), 7.10-7.05 (m, 3H), 6.08 (s, 1H), 3.86-3.82 (m, 1H), 3.39-3.35 (m, 1H), 3.10-3.06 (m, 1H), 2.91-2.85 (m, 9H), 1.62-1.58 (m, 1H), 1.50-1.26 (m, 7H).

The following compounds were prepared using general procedure GP-3:

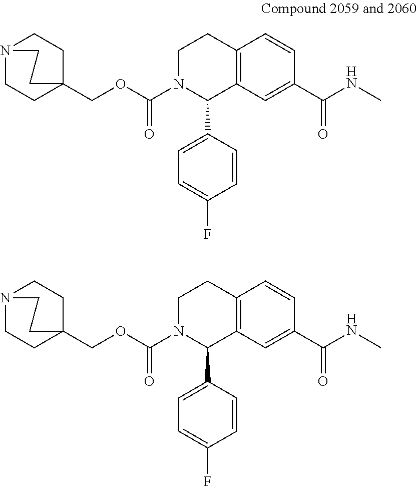 [SMILES: CNC(=O)c1ccc2c(c1)[C@H](c1ccc(F)cc1)N(C(=O)OCC13CCN(CC1)CC3)CC2 ; CNC(=O)c1ccc2c(c1)[C@@H](c1ccc(F)cc1)N(C(=O)OCC13CCN(CC1)CC3)CC2]

The diastereomers were separated by chiral SFC, eluting with n-hexane (0.1% v/v DEA):EtOH (0.1% v/v DEA), 10:90 from an EnantioPak® IG column (4.6×250 mm 5 μm) to afford Compound 2059 (retention time 14.03 min) and Compound 2060 (retention time 19.99 min). Stereochemical assignment at 1 position of the tetrahydroisoquinoline is based on chromatographic elution order as compared to diastereomers of related analogues of known configuration.

Compound 2059: LCMS: (M+H)⁺ 452; retention time 1.290 min. by CP Method E

¹H NMR (400 MHz, CD₃OD) δ 7.60 (d, J=7.6 Hz, 1H), 7.48 (s, 1H), 7.25 (d, J=8.0 Hz, 1H), 7.14 (s, 2H), 6.95 (t, J=8.4 Hz, 2H), 6.29 (s, 1H), 3.97-3.88 (m, 1H), 3.84 (s, 2H), 3.41-3.26 (m, 1H), 3.02-2.94 (m, 6H), 2.92-2.86 (m, 1H), 2.85-2.80 (m, 1H), 2.78 (s, 3H), 1.60-1.47 (m, 6H).

Compound 2060: LCMS: (M+H)⁺ 452; retention time 1.290 min. by CP Method E

¹H NMR (400 MHz, CD₃OD) δ 7.60 (d, J=7.6 Hz, 1H), 7.48 (s, 1H), 7.25 (d, J=8.0 Hz, 1H), 7.14 (s, 2H), 6.95 (t, J=8.4 Hz, 2H), 6.28 (s, 1H), 3.95-3.88 (m, 1H), 3.84 (s, 2H), 3.41-3.22 (m, 1H), 3.02-2.92 (m, 6H), 2.92-2.86 (m, 1H), 2.83-2.79 (m, 1H), 2.78 (s, 3H), 1.58-1.46 (m, 6H).

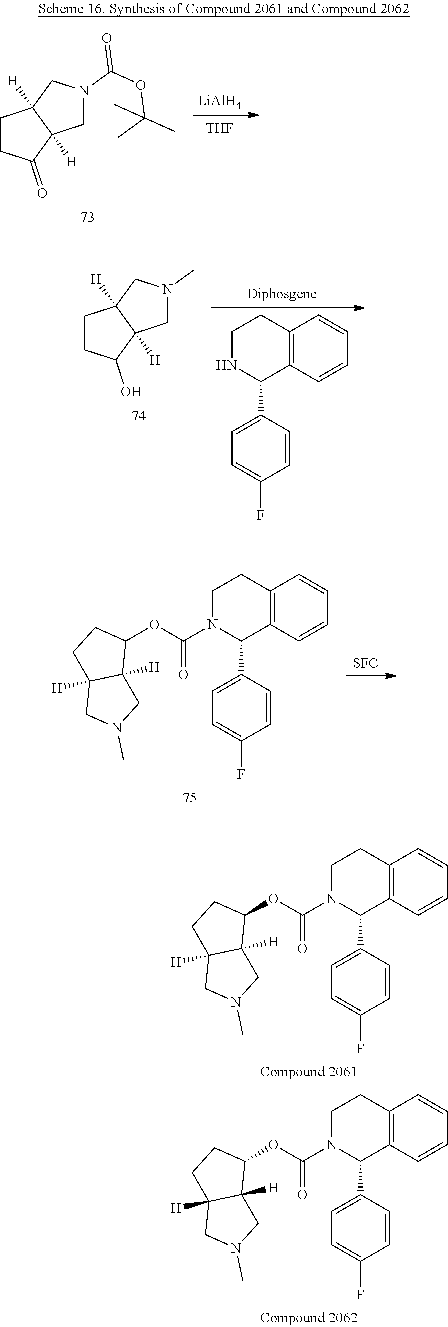

Step 1: To a solution of rel-(3aS,6aR)-tert-butyl-4-oxohexahydrocyclopenta[c]pyrrole-2(1H-carboxylate 73 (0.2 g, 0.89 mmol) in dry THF (5 mL) at 0° C. was added dropwise LiAlH₄ (2.66 mL, 2.66 mmol). After the addition was complete, the reaction mixture was heated under reflux for 16 hours. The reaction mixture was cooled, poured into water and washed with two 10 mL portions of DCM. The aqueous layer was concentrated and the residue suspended in a mixture of DCM and MeOH (30 mL, 5:1). Solids were removed by filtration and the filtrate was concentrated in vacuo to give crude rel-(3aS,6aR)-2-methyloctahydrocyclopenta[c]pyrrol-4-ol 74 (0.2 g) as a colorless oil.

LCMS: (M+H)⁺ 142; retention time=0.33 min. by CP Method B

Step 2: To a solution of crude rel-(3aS,6aR)-2-methyloctahydrocyclopenta[c]pyrrol-4-ol 74 (93 mg, 0.66 mmol) in dry CH₃CN (5 mL) was added diphosgene (65 mg, 0.33 mmol) at rt. The reaction mixture was stirred at 25° C. for 2 hours. The reaction mixture was concentrated and the residue dissolved in dry DMF (5 mL). To this solution was added (S)-1-(4-fluorophenyl)-1,2,3,4-tetrahydroisoquinoline 5 (50 mg, 0.22 mmol) and TEA (0.1 mL, 0.66 mmol). The reaction mixture was heated to 80° C. for 16 hours. After cooling, the reaction mixture was purified by prep-HPLC to give (1 S)-(rel-(3′aR,6′aS)-2′-methyloctahydrocyclopenta[c]pyrrol-4-yl)-1-(4-fluorophenyl)-3,4-dihydroisoquinoline-2(1H)-carboxylate 75 (20 mg, 0.05 mmol) as a light brown oil.

LCMS: (M+H)⁺ 395; retention time 1.494 min. by CP Method A

Step 3: The diastereomers were separated by chiral SFC eluting with EtOH containing 1% methanolic ammonia over an EnantioPak® AD-H column (4.6×250 mm 5 μm) to give Compound 2061 (retention time 1.19 min) and Compound 2062 (retention time 1.95 min). Stereochemical assignment of (S) at the 1 position of the tetrahydroisoquinoline is assigned based on chiral starting materials and stereochemical assignment of the pyrrolizidine is random based on chromatographic elution order.

Compound 2061: LCMS: (M+H)⁺ 395; Retention time 1.814 min. by CP Method B

¹H NMR (400 MHz, CD₃OD) δ 7.26-7.19 (m, 5H), 7.09-7.02 (m, 3H), 6.34 (s, 1H), 5.03 (d, J=6.4 Hz, 1H), 4.02-3.98 (m, 1H), 3.01-2.70 (m, 6H), 2.19-2.13 (m, 4H), 1.95-1.62 (m, 4H), 1.43-1.39 (m, 2H).

Compound 2062: LCMS: (M+H)⁺ 395; Retention time 1.806 min. by CP Method B

¹H NMR (400 MHz, CD₃OD) δ 7.25-7.20 (m, 5H), 7.10-7.06 (m, 3H), 6.37-6.19 (m, 1H), 5.01 (br, 1H), 4.03-3.98 (m, 1H), 3.00-2.65 (m, 6H), 2.44-2.24 (m, 4H), 2.04-1.82 (m, 4H), 1.69-1.26 (m, 2H).

The following compounds were prepared using General Procedure GP-1:

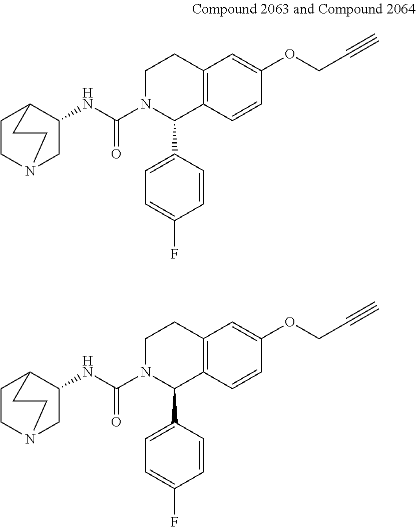 [SMILES: C#CCOc1ccc2c(c1)CCN(C(=O)N[C@@H]1CN3CCC1CC3)[C@H]2c1ccc(F)cc1 ; C#CCOc1ccc2c(c1)CCN(C(=O)N[C@@H]1CN3CCC1CC3)[C@@H]2c1ccc(F)cc1]

The diastereomers were separated by chiral SFC eluting with CO₂/MeOH containing 0.2% methanolic ammonia over an EnantioPak® IG column (4.6*100 mm 5 μm) to give Compound 2063 (retention time=2.51 min) and Compound 2064 (retention time=3.84 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2063: LCMS: (M+H)⁺=434.2; purity=100% (214 nm); retention time=1.538 min. CP Method B

¹H NMR (400 MHz, DMSO-d₆) δ 7.17-7.05 (m, 5H), 6.85-6.81 (m, 2H), 6.37 (s, 1H), 6.22 (d, J=6.4 Hz, 1H), 4.78 (d, J=2.4 Hz, 2H), 3.83-3.80 (m, 1H), 3.67-3.65 (m, 1H), 3.30 (s, 1H), 3.20-3.14 (m, 1H), 3.02-2.96 (m, 1H), 2.86-2.75 (m, 2H), 2.70-2.54 (m, 5H), 1.72-1.64 (m, 2H), 1.53-1.46 (m, 2H), 1.23-1.21 (m, 1H).

Compound 2064: LCMS: (M+H)⁺=343.3; purity=100% (214 nm); retention time=1.540 min. CP Method B

¹H NMR (400 MHz, DMSO-d₆) δ 7.16-7.09 (m, 5H), 6.86-6.81 (m, 2H), 6.41-6.39 (m, 2H), 4.78 (d, J=2.4 Hz, 2H), 3.83-3.77 (m, 2H), 3.23-3.16 (m, 2H), 2.92-2.65 (m, 8H), 1.87-1.82 (m, 2H), 1.66-1.62 (m, 2H), 1.39-1.35 (m, 1H)

The following compounds were prepared using General Procedure GP-3:

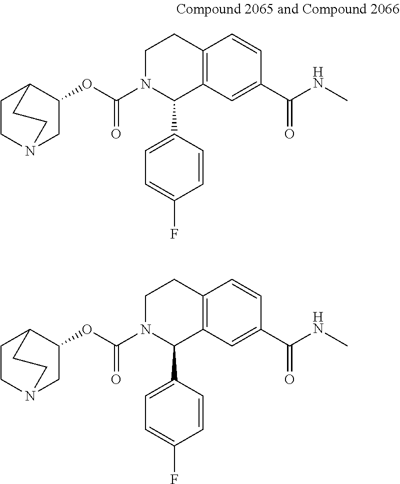

The diastereomers were separated by chiral SFC eluting with n-hexane (0.1% DEA):EtOH (0.1% DEA)=10:90 over an EnantioPak® IG column (4.6*250 mm 5 μm) to give Compound 2065 (retention time=19.03 min) and Compound 2066 (retention time=27.52 min). Stereochemical assignment of (S) at quinuclidine is absolute based on chiral starting materials, stereochemical assignment at 1 position of the tetrahydroisoquinoline is assigned based on chromatographic elution order as compared to diastereomers of related analogues of known configuration.

Compound 2065: LCMS: (M+H)⁺=438; purity=100% (214 nm); retention time=1.287 min. CP Method E

¹H NMR (400 MHz, DMSO-d₆) δ 8.36 (d, J=4.4 Hz, 1H), 7.70 (d, J=7.6 Hz, 1H), 7.61 (s, 1H), 7.33 (d, J=8.0 Hz, 1H), 7.24 (s, 2H), 7.16 (t, J=9.2 Hz, 2H), 6.31 (s, 1H), 4.66 (s, 1H), 3.97-3.87 (m, 1H), 3.17-3.07 (m, 2H), 2.98-2.84 (m, 2H), 2.73 (d, J=4.8 Hz, 4H), 2.63 (s, 3H), 1.98-1.91 (m, 1H), 1.78-1.54 (m, 2H), 1.53-1.42 (m, 1H), 1.38-1.28 (m, 1H).

Compound 2066: LCMS: (M+H)⁺=438; purity=100% (214 nm); retention time=1.481 min. CP Method C

¹H NMR (400 MHz, CD₃OD) δ 7.78-7.68 (m, 1H), 7.66-7.56 (m, 1H), 7.37 (d, J=9.2 Hz, 1H), 7.34-7.21 (m, 2H), 7.08 (s, 2H), 6.48-6.31 (m, 1H), 4.03 (s, 1H), 3.52-3.38 (m, 2H), 3.12-3.01 (m, 3H), 2.99-2.92 (m, 3H), 2.90 (s, 3H), 2.88-2.78 (m, 1H), 2.25-2.17 (m, 1H), 2.09-1.84 (m, 3H), 1.82-1.72 (m, 1H), 1.71-1.58 (m, 1H).

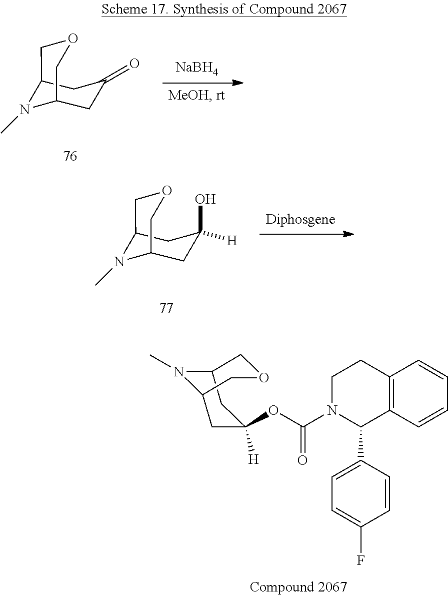

Step 1: To a solution of 9-methyl-3-oxa-9-azabicyclo[3.3.1]nonan-7-one 76 (0.5 g, 3.2 mmol) in MeOH (10 mL) was added NaBH₄ (0.38 g, 9.7 mmol) at 0° C. The reaction was stirred at rt for 16 h. The reaction was concentrated and the residue was dissolved in water (15 mL). The mixture was extracted with three 30 mL portions of DCM. The combined organic layers were dried and concentrated to give 9-methyl-3-oxa-9-azabicyclo[3.3.1]nonan-7-ol 77 (0.5 g, 3.2 mmol) as a white solid used in the next step without further purification.

LCMS: (M+1)⁺=158; Retention time=0.759 min. CP Method B

Step 2: To a solution of 9-methyl-3-oxa-9-azabicyclo[3.3.1]nonan-7-ol 77 (70 mg, 0.44 mmol) in dry CH₃CN (5 mL) was added diphosgene (0.08 mL, 0.66 mmol) at rt. The reaction was stirred at rt for 2 h. The reaction was concentrated and the residue was dissolved in dry DMF (5 mL). (S)-1-(4-fluorophenyl)-1,2,3,4-tetrahydroisoquinoline 5 (0.1 g, 0.44 mmol) and TEA (0.18 mL, 1.32 mmol) were added to the solution. The reaction mixture was heated to 80° C. for 16 h. After cooling, the reaction mixture was purified by prep-HPLC to give (1 S)-9′-methyl-3′-oxa-9′-azabicyclo[3.3.1]nonan-7′-yl-1-(4-fluorophenyl)-3,4-dihydroisoquinoline-2(1H)-carboxylate compound 2067 (36.6 mg, 0.9 mmol) as a light brown solid.

Compound 2067: LCMS: (M+1)⁺=411; Retention time=1.486 min. CP Method A

¹H NMR (400 Hz, CDCl₃): δ 7.26-7.18 (m, 5H), 7.07 (br, 1H), 6.97 (t, J=8.6 Hz, 2H), 6.65-6.47 (m, 1H), 5.23-5.17 (m, 1H), 4.29-4.10 (m, 1H), 3.99-3.95 (m, 2H), 3.70-3.57 (m, 2H), 3.24-2.98 (m, 2H), 2.80-2.66 (m, 3H), 2.54 (s, 3H), 2.42-2.37 (m, 2H), 1.68-1.59 (m, 2H).

The following compounds were prepared using General Procedure GP-1:

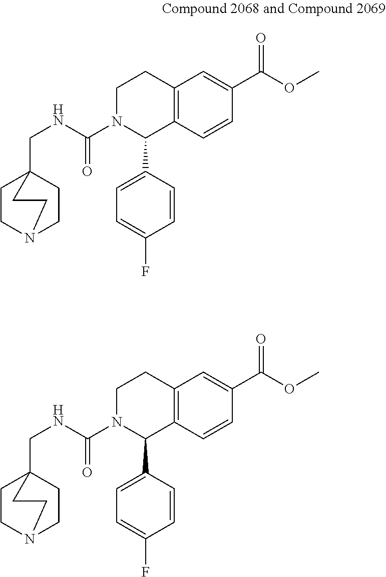 [SMILES: COC(=O)c1ccc2c(c1)CCN(C(=O)NCC13CCN(CC1)CC3)[C@H]2c1ccc(F)cc1 ; COC(=O)c1ccc2c(c1)CCN(C(=O)NCC13CCN(CC1)CC3)[C@@H]2c1ccc(F)cc1]

The diastereomers were separated by chiral SFC eluting with CO₂/MeOH containing 0.2% methanolic ammonia over an EnantioPak® IG column (20*250 mm 10 μm) to give Compound 2068 (retention time=1.3 min) and Compound 2069 (retention time=1.94 min). Stereochemical assignment at 1 position of the tetrahydroisoquinoline is assigned based on chromatographic elution order as compared to diastereomers of related analogues of known configuration.

Compound 2068: LCMS: (M+H)⁺=452; purity=93% (214 nm); retention time=1.666 min. CP Method A

¹H NMR (400 MHz, CD₃OD) δ 7.93 (s, 1H), 7.88 (d, J=8.0 Hz, 1H), 7.30 (d, J=8.0 Hz, 1H), 7.22 (dd, J=8.8, 5.6 Hz, 2H), 7.05 (t, J=8.8 Hz, 2H), 6.52 (s, 1H), 3.92 (s, 3H), 3.79 (dt, J=13.2, 5.6 Hz, 1H), 3.53-3.44 (m, 1H), 3.15-2.97 (m, 3H), 2.91-2.79 (m, 7H), 1.46-1.37 (m, 6H).

Compound 2069: LCMS: (M+H)⁺=452; purity=98% (214 nm); retention time=1.665 min. CP Method A

¹H NMR (400 MHz, CD₃OD) δ 7.94 (s, 1H), 7.89 (dd, J=8.0, 1.2 Hz, 1H), 7.30 (d, J=8.0 Hz, 1H), 7.21 (dd, J=8.4, 5.6 Hz, 2H), 7.04 (t, J=8.8 Hz, 2H), 6.55 (s, 1H), 3.93 (s, 3H), 3.78 (dt, J=13.2, 5.6 Hz, 1H), 3.52-3.45 (m, 1H), 3.35-3.15 (m, 8H), 3.09-2.98 (m, 1H), 2.87 (dt, J=16.4, 4.2 Hz, 1H), 1.81-1.72 (m, 6H).

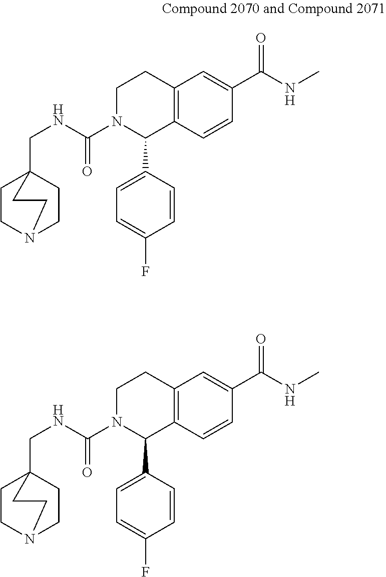 [SMILES: CNC(=O)c1ccc2c(c1)CCN(C(=O)NCC13CCN(CC1)CC3)[C@H]2c1ccc(F)cc1 ; CNC(=O)c1ccc2c(c1)CCN(C(=O)NCC13CCN(CC1)CC3)[C@@H]2c1ccc(F)cc1]

The diastereomers were separated by chiral SFC eluting with CO₂/MeOH containing 0.2% methanolic ammonia over an EnantioPak® IG column (4.6*100 mm 5 μm) to give Compound 2070 (retention time=1.92 min) and Compound 2071 (retention time=2.8 min). Stereochemical assignment at 1 position of the tetrahydroisoquinoline is assigned based on chromatographic elution order as compared to diastereomers of related analogues of known configuration.

Compound 2070: LCMS: (M+H)⁺=451; purity=99% (214 nm); retention time=1.394 min. CP Method A2

¹H NMR (400 MHz, CD₃OD) δ 7.74-7.65 (m, 2H), 7.32-7.18 (m, 3H), 7.08-7.01 (m, 2H), 6.50 (s, 1H), 3.82-3.75 (m, 1H), 3.52-3.44 (m, 1H), 3.16-3.08 (m, 1H), 3.04-2.98 (m, 2H), 2.94 (s, 3H), 2.89-2.81 (m, 7H), 1.47-1.38 (m, 6H).

Compound 2071: LCMS: (M+H)⁺=451; purity=100% (214 nm); retention time=1.396 min. CP Method A2

¹H NMR (400 MHz, CD₃OD) δ 7.61-7.53 (m, 2H), 7.17-7.06 (m, 3H), 6.96-6.88 (m, 2H), 6.38 (s, 1H), 3.71-3.63 (m, 1H), 3.41-3.32 (m, 1H), 3.03-2.94 (m, 1H), 2.92-2.84 (m, 2H), 2.82 (s, 3H), 2.76-2.69 (m, 7H), 1.34-1.26 (m, 6H).

The following compounds were prepared using General Procedure GP-4:

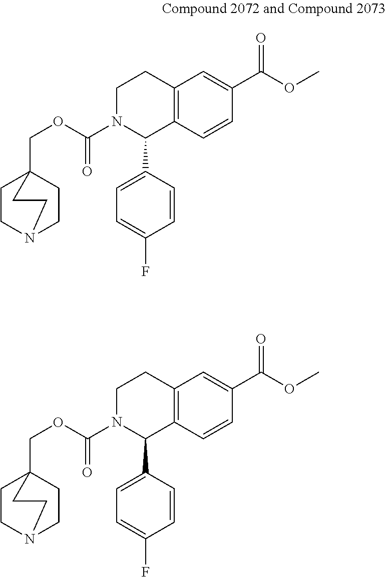 [SMILES: COC(=O)c1ccc2c(c1)CCN(C(=O)OCC13CCN(CC1)CC3)[C@H]2c1ccc(F)cc1 ; COC(=O)c1ccc2c(c1)CCN(C(=O)OCC13CCN(CC1)CC3)[C@@H]2c1ccc(F)cc1]

The diastereomers were separated by chiral SFC eluting with CO₂/EtOH containing 1% methanolic ammonia over an EnantioPak® AD column (20*250 mm 10 μm) to give Compound 2072 (retention time=1.8 min) and Compound 2073 (retention time=2.15 min). Stereochemical assignment at 1 position of the tetrahydroisoquinoline is assigned based on chromatographic elution order as compared to diastereomers of related analogues of known configuration.

Compound 2072: LCMS: (M+H)⁺=453; purity=94% (214 nm); retention time=1.735 min. CP Method A

¹H NMR (400 MHz, CD₃OD) δ 7.93 (s, 1H), 7.84 (d, J=8.0 Hz, 1H), 7.25 (s, 3H), 7.06 (t, J=8.4 Hz, 2H), 6.39 (s, 1H), 4.08-4.00 (m, 1H), 3.92 (s, 3H), 3.91-3.84 (m, 2H), 3.40 (brs, 1H), 3.08-2.98 (m, 1H), 2.96-2.84 (m, 7H), 1.50 (s, 6H).

Compound 2073: LCMS: (M+H)⁺=453; purity=98% (214 nm); retention time=1.741 min. CP Method A

¹H NMR (400 MHz, CD₃OD) δ 7.92 (s, 1H), 7.84 (d, J=8.4 Hz, 1H), 7.24 (s, 3H), 7.06 (t, J=8.0 Hz, 2H), 6.38 (s, 1H), 4.08-4.00 (m, 1H), 3.91 (s, 3H), 3.91-3.84 (m, 2H), 3.40 (brs, 1H), 3.07-2.96 (m, 1H), 2.96-2.88 (m, 7H), 1.52 (s, 6H).

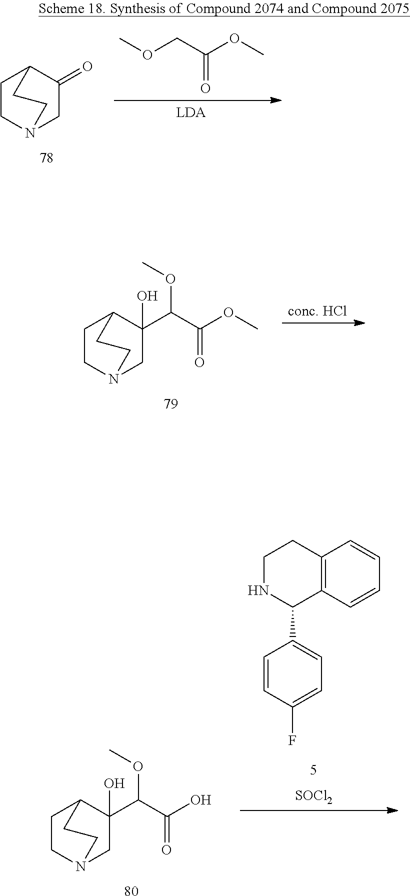

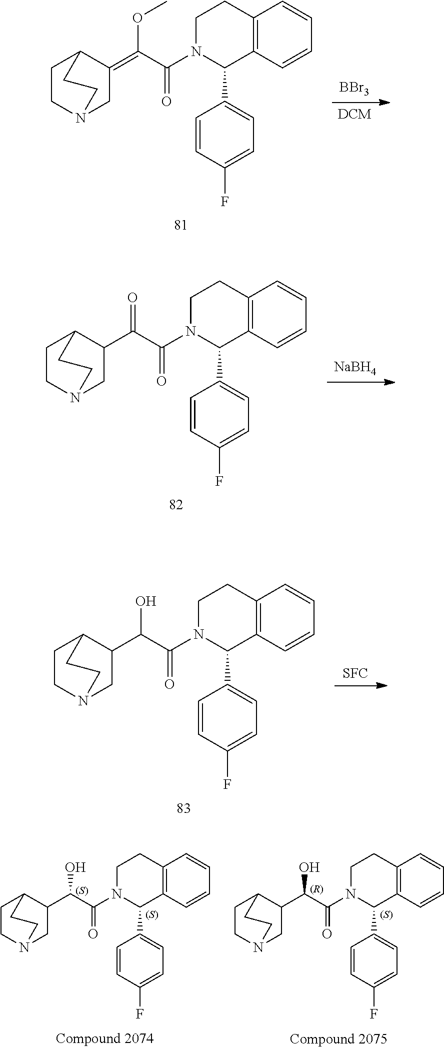

Step 1: To a mixture of methyl 2-methoxyacetate (3.74 g, 36 mmol) dissolved in THF (50 mL) was added LDA (18 mL, 36 mmol) dropwise at −78° C. The reaction mixture was stirred at −78° C. for 1 hr, then quinuclidin-3-one 78 (1.5 g, 12 mmol) in THF (50 mL) was added dropwise. The resulting mixture was stirred at 25° C. for 16 hr. The reaction mixture was diluted with 100 mL of water and extracted with three 50 mL portions of EA. The combined organic phases were washed by brine (100 mL), dried and concentrated to give a crude oil 79 (0.8 g), which was used for the next step directly without further purification.

LCMS: r.t.: 0.76 min, [M+H]⁺=230

Step 2: HCl (excess) was charged into a round-bottom flask and ester 79 (100 mg, 0.43 mmol) was added. The mixture was stirred at 25° C. for 2 hours. The reaction mixture was concentrated to give a crude oil 80 (0.8 g), which was used for the next step directly without further purification.

LCMS: r.t.: 0.36 min, [M+H]⁺=216

Step 3: 2-(3-hydroxyquinuclidin-3-yl)-2-methoxyacetic acid 80 (400 mg, 2.03 mmol, 1 equiv) was dissolved in SOCl₂ (10 mL) and stirred for 3 hr. The reaction was concentrated and the residue dissolved in DCM (10 mL). The mixture was added into (S)-1-(4-fluorophenyl)-1,2,3,4-tetrahydroisoquinoline 5 (452 mg, 2.03 mmol, 1.0 equiv) and TEA (1.14 g, 3.05 mmol, 1.5 equiv) in DCM (10 mL). The mixture was stirred at 25° C. for 1 hour. To the mixture was added water (30 mL) and it was extracted with three 20 mL portions of EA. The combined organic layers were washed with brine, dried and concentrated in vacuo to give a crude oil, which was purified by prep-HPLC to afford (S, E)-1-(1-(4-fluorophenyl)-3,4-dihydroisoquinolin-2(1H)-yl)-2-methoxy-2-(quinuclidin-3-ylidene)ethanone 81 (100 mg) as a yellow solid.

LCMS: r.t.: 1.67 min, [M+H]⁺=394, purity: 95%

Step 4: To a solution of (S, E)-1-(1-(4-fluorophenyl)-3, 4-dihydroisoquinolin-2(1H)-yl)-2-methoxy-2-(quinuclidin-3-ylidene) ethanone 81 (100 mg, 0.25 mmol) in DCM (5 mL) was added BBr₃ (0.25 mL, 0.25 mmol) at r.t. under nitrogen atmosphere. The mixture was stirred at r.t for 3 h. The reaction mixture was quenched by water, extracted by EA. The organic phase was washed by brine, dried and concentrated to obtain (S, E)-1-(1-(4-fluorophenyl)-3, 4-dihydroisoquinolin-2(1H)-yl)-2-hydroxy-2-(quinuclidin-3-ylidene) ethanone 82 (66 mg).

LCMS: r.t.: 1.46 min, [M+H]⁺=393

Step 5 To a solution of (S, E)-1-(1-(4-fluorophenyl)-3, 4-dihydroisoquinolin-2(1H)-yl)-2-hydroxy-2-(quinuclidin-3-ylidene) ethanone 82 (66 mg, 0.16 mmol) in MeOH (5 mL) was added NaBH₄ (10 mg, 0.25 mmol) at r.t. The mixture was stirred at r.t for 0.5 h. The reaction mixture was concentrated and diluted with water (10 mL) and extracted with three 10 mL portions of EA. The combined organic layers were washed by brine (20 mL), dried over anhydrous Na₂SO₄, filtered and concentrated to obtain a crude oil, which was purified by prep-HPLC to afford 1-((S)-1-(4-fluorophenyl)-3,4-dihydroisoquinolin-2(1H)-yl)-2-hydroxy-2-(quinuclidin-3-yl)ethanone 83 (40 mg).

The diastereomers were separated by chiral SFC eluting with n-Hexane (0.1% DEA):EtOH (0.1% DEA)=10:90 over an EnantioPak® IG column (4.6*250 mm 5 μm) to give Compound 2074 (retention time=7.441 min) and Compound 2075 (retention time=10.509 min). Stereochemical assignment of (S) at quinuclidine is absolute based on starting material, stereochemical assignment at 1 position of the tetrahydroisoquinoline is assigned based on chromatographic elution order as compared to diastereomers of related analogues of known configuration.

Compound 2074: LCMS: (M+H)⁺=395, purity=100% (214 nm), Retention time=1.463 min. CP Method E

¹H NMR (400 MHz, DMSO-d₆) δ 7.40-7.20 (m, 4H), 7.20-7.05 (m, 4H), 6.59 (d, J=50.8 Hz, 1H), 5.67 (d, J=6.9 Hz, 1H), 4.30-4.10 (m, 2H), 3.06-2.96 (m, 1H), 2.81 (s, 1H), 2.69 (d, J=8.2 Hz, 4H), 2.13 (d, J=13.5 Hz, 1H), 1.99 (dd, J=20.2, 12.5 Hz, 2H), 1.71 (s, 1H), 1.57 (s, 1H), 1.47 (s, 2H), 1.38-1.27 (m, 2H).

Compound 2075: LCMS: (M+H)⁺=395, purity=100% (214 nm), Retention time=1.470 min. CP Method E

¹H NMR (400 MHz, DMSO-d₆) δ 7.42-7.00 (m, 8H), 6.73 (s, 1H), 5.55 (d, J=8.1 Hz, 1H), 4.37-4.27 (m, 1H), 4.12 (d, J=11.3 Hz, 1H), 3.04 (s, 1H), 2.94 (s, 1H), 2.87-2.72 (m, 4H), 2.26-1.99 (m, 3H), 1.98 (s, 1H), 1.80 (s, 1H), 1.64 (d, J=8.2 Hz, 1H), 1.54 (s, 1H), 1.34 (dd, J=17.6, 11.6 Hz, 2H).

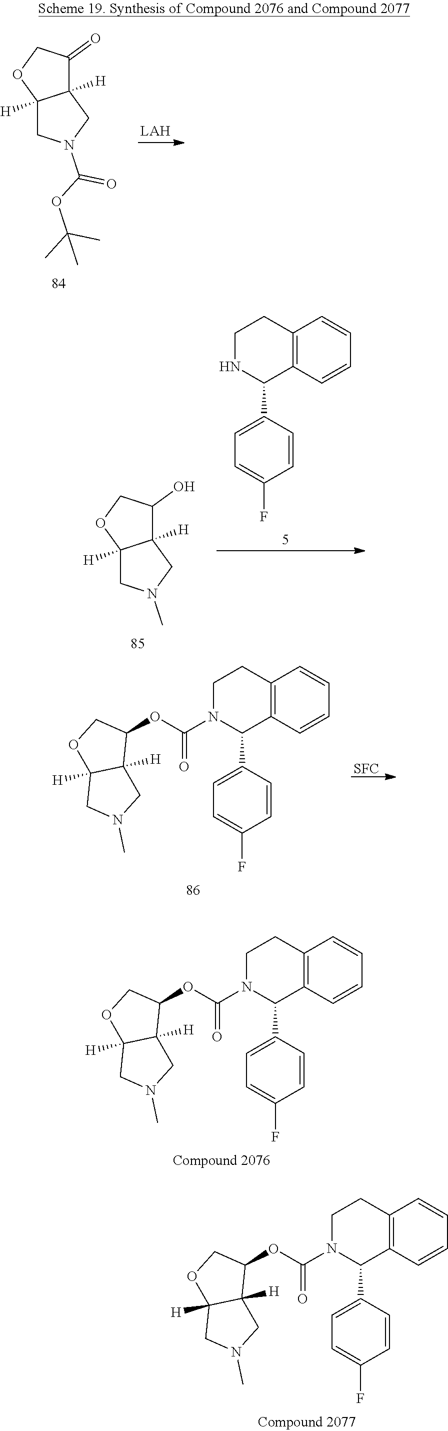

Step 1: To a solution of (3aS, 6aS)-tert-butyl 3-oxotetrahydro-2H-furo[2,3-c]pyrrole-5(3H)-carboxylate (227 mg, 1 mmol) in THF (2 mL) was added LAH (1 M in THF, 2 mL) at −70° C. Then the mixture was stirred at room temperature for 0.5 h. The mixture was quenched by saturated aqueous Na₂SO₄, filtered and the solid was washed by THF (10 mL). The filtrate was concentrated in vacuo to give (3aR, 6aS)-5-methylhexahydro-2H-furo[2,3-c]pyrrol-3-ol 85 (110 mg) as a light yellow solid.

Step 2: To a solution of (3aR, 6aS)-5-methylhexahydro-2H-furo[2,3-c]pyrrol-3-ol 85 (110 mg, 0.77 mmol) in MeCN (3 mL) was added trichloromethyl carbonochloridate (152 mg, 0.77 mmol) and the mixture was stirred at room temperature for 2 h. The mixture was concentrated to give a white solid. The white solid was dissolved in 3 mL of DMF and (S)-1-(4-fluorophenyl)-1, 2, 3, 4-tetrahydroisoquinoline 5 (175 mg, 0.77 mmol), TEA (156 mg, 1.54 mmol) was added. The mixture was stirred at 90° C. overnight. The mixture was cooled to 25° C. and water (10 mL) was added. The mixture was extracted with three 10 mL portions of ethyl acetate. The combined organic layers were washed with brine, dried and concentrated in vacuo to give crude product. The crude product was purified by column chromatography eluting with PE/EA (3:1) to give 15 mg of (1S)-((3′aS,6′aS)-5-methylhexahydro-2H-furo[2,3-c]pyrrol-3-yl) 1-(4-fluorophenyl)-3,4-dihydroisoquinoline-2(1H)-carboxylate 86 as a white solid.

The diastereomers were separated by chiral SFC eluting with n-hexane (0.1% DEA):EtOH (0.1% DEA)=10:90 over an EnantioPak® Cellulose-SC (4.6*100 mm Sum) to give Compound 2076 (retention time=1.46 min) and Compound 2077 (retention time=1.97 min). Stereochemical assignment of (S) at the 1 position of the tetrahydroisoquinoline is assigned based on chiral starting materials.

Compound 2076: LCMS: (M+H)⁺=397; purity=95.9% (214 nm); Retention time=1.844 min. CP Method A2

¹H NMR (400 MHz, CDCl₃) δ 7.17 (ddd, J=13.8, 9.6, 6.6 Hz, 5H), 7.03-6.89 (m, 3H), 6.23 (s, 1H), 4.59-4.52 (m, 1H), 4.14-4.08 (m, 1H), 3.92 (dd, J=9.1, 6.3 Hz, 1H), 3.86 (dd, J=11.2, 4.9 Hz, 1H), 3.81-3.74 (m, 2H), 3.56-3.49 (m, 2H), 3.39 (s, 2H), 3.32 (d, J=5.1 Hz, 3H), 3.01-2.94 (m, 2H), 2.82 (dd, J=11.8, 8.0 Hz, 1H).

Compound 2077: LCMS: (M+H)⁺=458; purity=100% (214 nm); Retention time=1.844 min. CP Method A2

¹H NMR (400 MHz, CDCl₃) δ 7.26-7.17 (m, 5H), 7.09-6.86 (m, 3H), 6.23 (s, 1H), 4.51-4.48 (m, 1H), 4.02-3.87 (dd, J=9.2, 6.4 Hz, 1H), 3.74 (dd, J=9.1, 6.3 Hz, 1H), 3.72-3.68 (m, 3H), 3.59 (d, J=12.0, 1H), 3.35-3.23 (m, 3H), 3.32 (d, J=5.1 Hz, 3H), 2.99-2.88 (m, 2H), 2.77 (dd, J=11.8, 8.0 Hz, 1H).

The following compounds were prepared by General Procedure GP-4:

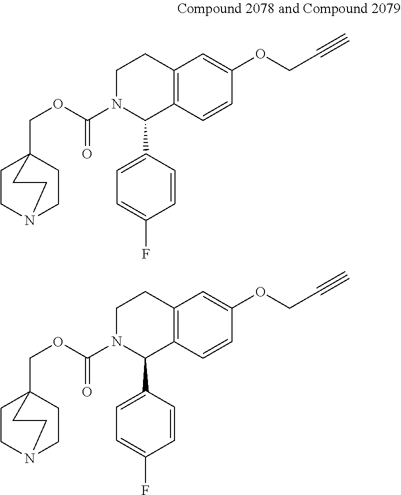

The diastereomers were separated by chiral SFC eluting with CO₂/MeOH containing 0.2% methanolic ammonia over an EnantioPak® IG column (4.6*100 mm 5 μm) to give Compound 2078 (retention time=3.23 min) and Compound 2079 (retention time=3.95 min). Stereochemical assignment at 1 position of the tetrahydroisoquinoline is assigned based on chromatographic elution order as compared to diastereomers of related analogues of known configuration.

Compound 2078: LCMS: (M+H)⁺=449.2; purity=100% (214 nm); retention time=1.502 min. CP Method B

¹H NMR (400 MHz, CDCl₃) δ 7.19-7.16 (m, 2H), 6.99-6.94 (m, 3H), 6.84-6.80 (m, 2H), 6.36-6.17 (m, 1H), 4.72 (s, 2H), 4.06-3.97 (m, 1H), 3.87-3.79 (m, 2H), 3.24-3.17 (m, 1H), 2.96-2.87 (m, 7H), 2.77-2.71 (m, 1H), 2.54-2.53 (m, 1H), 1.43-1.39 (m, 6H).

Compound 2079: LCMS: (M+H)⁺=449.2; purity=100% (214 nm); retention time=1.505 min. CP Method B

¹H NMR (400 MHz, CDCl₃) δ 7.18-7.16 (m, 2H), 6.99-6.94 (m, 3H), 6.84-6.80 (m, 2H), 6.37-6.17 (m, 1H), 4.69 (s, 2H), 4.06-3.97 (m, 1H), 3.87-3.79 (m, 2H), 3.24-3.17 (m, 1H), 2.89 (t, J=2.8 Hz, 7H), 2.77-2.72 (m, 1H), 2.53 (t, J=2.4 Hz, 1H), 1.43-1.39 (m, 6H).

The following compounds were prepared by General Procedure GP-3:

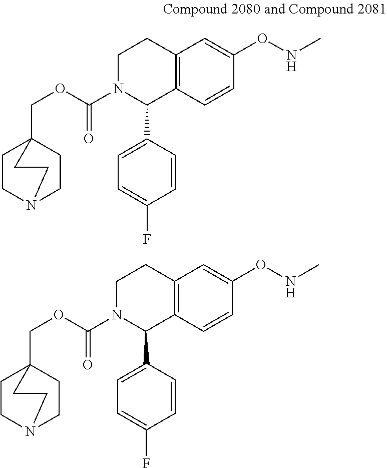

The diastereomers were separated by chiral SFC eluting with n-hexane (0.1% DEA):EtOH (0.1% DEA)=50:50 over an EnantioPak® IG column (4.6*250 mm 5 μm) to give Compound 2080 (retention time=9.40 min) and Compound 2081 (retention time=11.35 min). Stereochemical assignment at 1 position of the tetrahydroisoquinoline is assigned based on chromatographic elution order as compared to diastereomers of related analogues of known configuration.

Compound 2080: LCMS: (M+H)⁺=452; purity=100% (254 nm); retention time=1.276 min. CP Method E

¹H NMR (400 MHz, CD₃OD) δ 7.72 (s, 1H), 7.65 (d, J=8.0 Hz, 1H), 7.38-7.28 (m, 3H), 7.17-7.02 (m, 2H), 6.37 (s, 1H), 4.04-3.98 (m, 1H), 3.97-3.86 (m, 2H), 3.52-3.36 (m, 1H), 3.09-2.98 (m, 7H), 2.97-2.88 (m, 4H), 1.68-1.46 (m, 6H).

Compound 2081: LCMS: (M+H)⁺=452; purity=100% (254 nm); retention time=1.388 min. CP Method C

¹H NMR (400 MHz, CD₃OD) δ 7.72 (s, 1H), 7.65 (d, J=8.0 Hz, 1H), 7.35-7.17 (m, 3H), 7.12-7.02 (m, 2H), 6.37 (s, 1H), 4.12-3.98 (m, 1H), 3.94-3.84 (m, 2H), 3.56-3.36 (m, 3H), 3.10-2.98 (m, 2H), 2.97-2.93 (m, 4H), 2.92-2.88 (m, 3H), 1.58-1.42 (m, 6H).

The following compounds were prepared by General Procedure GP-4:

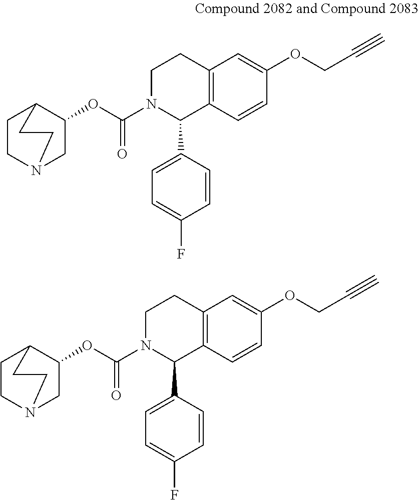

The diastereomers were separated by chiral SFC eluting with CO₂/MeOH containing 0.2% methanolic ammonia over an EnantioPak® IG column (4.6*100 mm 5 μm) to give Compound 2082 (retention time=1.91 min) and Compound 2083 (retention time=2.69 min). Stereochemical assignment of (S) at quinuclidine is absolute based on starting materials; stereochemical assignment at 1 position of the tetrahydroisoquinoline is assigned based on chromatographic elution order as compared to diastereomers of related analogues of known configuration.

Compound 2082: LCMS: (M+H)⁺=435.3; purity=100% (214 nm); retention time=1.598 min. CP Method B

¹H NMR (400 MHz, CDCl₃) δ 7.18-7.15 (m, 2H), 6.98 (t, J=8.8 Hz, 2H), 6.85-6.80 (m, 2H), 6.35-6.06 (m, 1H), 4.97 (brs, 1H), 4.69 (d, J=2.4 Hz, 2H), 4.14-3.73 (m, 1H), 3.49-3.26 (m, 1H), 3.17-3.07 (m, 5H), 3.00-2.87 (m, 1H), 2.80-2.76 (m, 1H), 2.54-2.53 (m, 1H), 2.36 (s, 1H), 1.92 (s, 1H), 1.85-1.80 (m, 1H), 1.70-1.61 (m, 1H), 1.26 (t, J=7.2 Hz, 2H).

Compound 2083: LCMS: (M+H)⁺=435.3; purity=94.19% (214 nm); retention time=1.503 min. CP Method B

¹H NMR (400 MHz, CDCl₃) δ 7.21-7.17 (m, 2H), 6.98-6.94 (m, 3H), 6.84-6.80 (m, 2H), 6.37-6.18 (m, 1H), 4.81-4.79 (brs, 1H), 4.69 (d, J=2.4 Hz, 2H), 4.07-3.95 (m, 1H), 3.92-3.20 (m, 2H), 3.01-2.93 (m, 1H), 2.89-2.85 (m, 2H), 2.83-2.70 (m, 4H), 2.53 (d, J=2.4 Hz 1H), 2.05-2.03 (m, 1H), 1.85-1.78 (m, 1H), 1.61-1.52 (m, 1H), 1.46-1.39 (m, 1H), 1.29-1.26 (m, 1H).

The following compounds were prepared using General Procedure GP-1

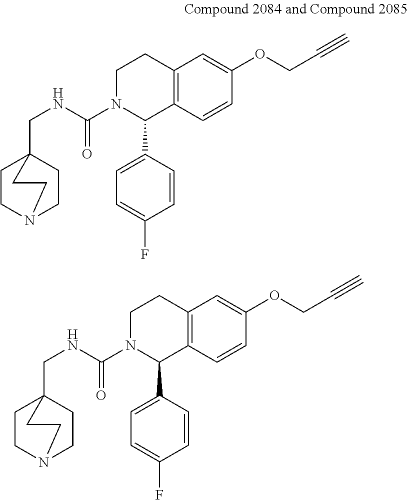

The diastereomers were separated by chiral SFC eluting with CO₂/EtOH containing 1% methanol/ammonia over an EnantioPak® IG column (20×250 mm, 10 μm) to give Compound 2084 (retention time 2.35 min) and Compound 2085 (retention time 3.01 min). Stereochemical assignment at the 1 position of the tetrahydroisoquinoline is assigned based on chromatographic elution order as compared to diastereomers of related analogues of known configuration.

Compound 2084: LCMS: (M+H)⁺ 448.2; purity 99.73% (214 nm); retention time 1.484 min. by CP Method F

¹H NMR (400 MHz, CDCl₃) δ 7.22-7.19 (m, 2H), 7.07 (d, J=8.4 Hz, 1H), 6.97 (t, J=8.8 Hz, 2H), 6.85-6.79 (m, 2H), 6.24 (s, 1H), 4.68 (d, J=2.4 Hz, 2H), 4.44 (t, J=6 Hz, 1H), 3.61-3.57 (m, 2H), 3.15-3.10 (m, 1H), 3.02-2.97 (m, 1H), 2.91-2.76 (m, 8H), 2.53 (t, J=2 Hz, 1H), 1.30-1.26 (m, 6H).

Compound 2085: LCMS: (M+H)⁺=448.2; purity=98.76% (214 nm); retention time=1.491 min. CP Method F

¹H NMR (400 MHz, CDCl₃) δ 7.21-7.17 (m, 2H), 7.07 (d, J=8.4 Hz, 1H), 6.97 (t, J=8.4 Hz, 2H), 6.86-6.80 (m, 2H), 6.25 (s, 1H), 4.69 (d, J=2 Hz, 2H), 4.61 (t, J=6.4 Hz, 1H), 3.64-3.53 (m, 2H), 3.28-3.23 (m, 1H), 3.15-3.08 (m, 7H), 2.93-2.77 (m, 2H), 2.54 (t, J=2.4 Hz, 1H), 1.58-1.54 (m, 6H).

The following compounds were prepared by General Procedure GP-4:

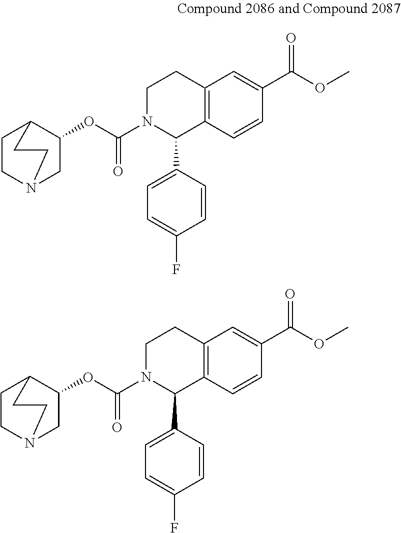 [SMILES: COC(=O)c1ccc2c(c1)CCN(C(=O)O[C@@H]1CN3CCC1CC3)[C@H]2c1ccc(F)cc1 ; COC(=O)c1ccc2c(c1)CCN(C(=O)O[C@@H]1CN3CCC1CC3)[C@@H]2c1ccc(F)cc1]

The diastereomers were separated by chiral SFC eluting with CO₂/MeOH containing 0.2% methanol/ammonia over an EnantioPak® AD column (20×250 mm, 10 μm) to give Compound 2086 (retention time 1.86 min) and Compound 2087 (retention time 2.41 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2086: LCMS: (M+H)⁺ 439; purity 97% (214 nm); retention time 1.747 min. by CP Method A

¹H NMR (400 MHz, CD₃OD) δ 7.94 (s, 1H), 7.85 (d, J=8.0, 1H), 7.36-7.20 (m, 3H), 7.07 (t, J=7.2 Hz, 2H), 6.48-6.32 (br, 1H), 5.08 (s, 1H), 4.10 (dt, J=17.2, 4.8 Hz, 1H), 3.92 (s, 3H), 3.72 (dd, J=13.6, 10.0 Hz, 1H), 3.32-3.20 (m, 5H), 3.10-3.01 (m, 1H), 3.00-2.90 (br, 1H), 2.41 (s, 1H), 2.25-2.02 (m, 2H), 2.01-1.82 (m, 1H).

Compound 2087: LCMS: (M+H)⁺ 439; purity 99% (214 nm); retention time 1.754 min. by CP Method A

¹H NMR (400 MHz, CD₃OD) δ 7.94 (s, 1H), 7.85 (d, J=8.0, 1H), 7.26 (s, 3H), 7.07 (t, J=7.2 Hz, 2H), 6.41 (br s, 1H), 4.87-4.81 (m, 1H), 4.04 (s, 1H), 3.92 (s, 3H), 3.60-3.34 (br, 1H), 3.26 (dd, J=14.4, 8.0 Hz, 1H), 3.10-2.68 (m, 7H), 1.94-1.74 (m, 2H), 1.72-1.60 (m, 1H), 1.59-1.48 (m, 1H).

The following compounds were prepared using General Procedure GP-1:

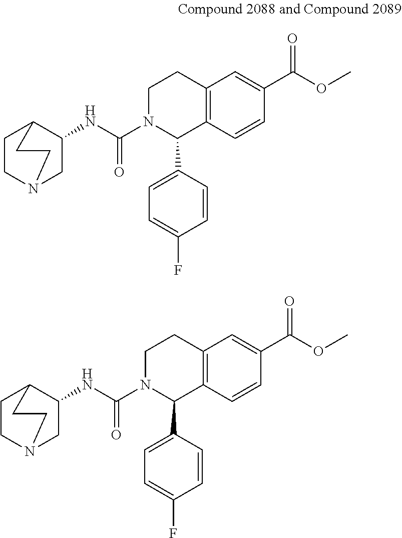 [SMILES: COC(=O)c1ccc2c(c1)CCN(C(=O)N[C@@H]1CN3CCC1CC3)[C@H]2c1ccc(F)cc1 ; COC(=O)c1ccc2c(c1)CCN(C(=O)N[C@@H]1CN3CCC1CC3)[C@@H]2c1ccc(F)cc1]

The diastereomers were separated by chiral SFC eluting with CO₂/MeOH containing 0.2% methanol/ammonia over an EnantioPak® AD column (20×250 mm, 10 μm) to give Compound 2088 (retention time 4.33 min) and Compound 2089 (retention time 2.63 min). Stereochemical assignment of (S) at quinuclidine is absolute based on starting materials, stereochemical assignment at the 1 position of the tetrahydroisoquinoline is assigned based on chromatographic elution order as compared to diastereomers of related analogues of known configuration.

Compound 2088: LCMS: (M+H)⁺ 438; purity 94% (214 nm); retention time 1.680 min. by CP Method A

¹H NMR (400 MHz, CD₃OD) δ 7.93 (s, 1H), 7.87 (dd, J=8.0, 1.6 Hz, 1H), 7.27 (d, J=8.0 Hz, 1H), 7.20 (dd, J=8.4, 5.2 Hz, 2H), 7.04 (t, J=8.8 Hz, 2H), 6.57 (s, 1H), 3.92 (s, 3H), 3.91-3.84 (m, 2H), 3.50-3.42 (m, 1H), 3.26 (ddd, J=13.6, 9.6, 2.0 Hz, 1H), 3.09-2.99 (m, 1H), 2.94-2.76 (m, 5H), 2.66 (ddd, J=14.0, 5.6, 2.0 Hz, 1H), 1.96 (q, J=2.8 Hz, 1H), 1.92-1.81 (m, 1H), 1.80-1.70 (m, 2H), 1.56-1.46 (m, 1H).

Compound 2089: LCMS: (M+H)⁺ 438; purity 95% (214 nm); retention time 1.676 min. by CP Method A

¹H NMR (400 MHz, CD₃OD) δ 7.92 (s, 1H), 7.87 (dd, J=8.0, 1.2 Hz, 1H), 7.28 (d, J=8.0 Hz, 1H), 7.21 (dd, J=8.4, 5.2 Hz, 2H), 7.04 (t, J=8.8 Hz, 2H), 6.56 (s, 1H), 3.92 (s, 4H), 3.90-3.83 (m, 1H), 3.50-3.42 (m, 1H), 3.27 (ddd, J=13.6, 9.6, 2.0 Hz, 1H), 3.08-2.76 (m, 6H), 2.72 (ddd, J=14.0, 5.6, 2.0 Hz, 1H), 1.92 (q, J=3.2 Hz, 1H), 1.85-1.70 (m, 3H), 1.54-1.44 (m, 1H).

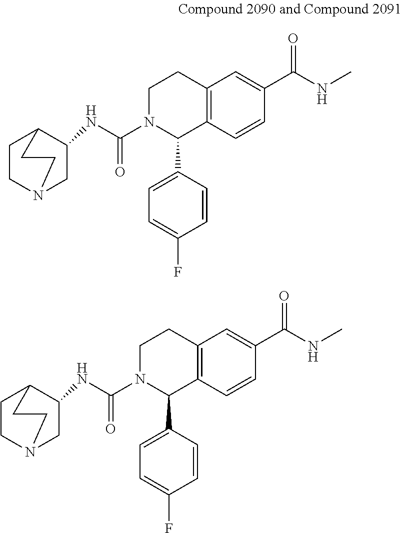 [SMILES: CNC(=O)c1ccc2c(c1)CCN(C(=O)N[C@@H]1CN3CCC1CC3)[C@H]2c1ccc(F)cc1 ; CNC(=O)c1ccc2c(c1)CCN(C(=O)N[C@@H]1CN3CCC1CC3)[C@@H]2c1ccc(F)cc1]

The diastereomers were separated by chiral SFC eluting with CO₂/MeOH containing 0.2% methanol/ammonia over an EnantioPak® IG column (4.6×100 mm, 5 μm) to give Compound 2090 (retention time 2.33 min) and Compound 2091 (retention time 3.61 min). Stereochemical assignment of (S) at quinuclidine is absolute based on starting materials, stereochemical assignment at 1 position of the tetrahydroisoquinoline is assigned based on chromatographic elution order as compared to diastereomers of related analogues of known configuration.

Compound 2090: LCMS: (M+H)⁺ 437; purity 99% (214 nm); retention time 1.332 min. by CP Method C

¹H NMR (400 MHz, CDCl₃) δ 7.67 (s, 1H), 7.54 (d, J=8.0 Hz, 1H), 7.22-7.14 (m, 3H), 7.02-6.92 (m, 2H), 6.43 (s, 1H), 6.16-6.06 (br, 1H), 4.64 (d, J=5.6 Hz, 1H), 3.92-3.82 (m, 1H), 3.66-3.52 (m, 2H), 3.38-3.29 (m, 1H), 3.03 (d, J=4.8 Hz, 3H), 2.99-2.92 (m, 1H), 2.91-2.84 (m, 1H), 2.83-2.71 (m, 4H), 2.38 (d, J=10.4 Hz, 1H), 1.95-1.88 (m, 1H), 1.71-1.63 (m, 2H), 1.52-1.41 (m, 2H).

Compound 2091: LCMS: (M+H)⁺ 437; purity 98% (214 nm); retention time 1.310 min. by CP Method C

¹H NMR (400 MHz, CD₃OD) δ 7.77 (s, 1H), 7.65 (d, J=8.4 Hz, 1H), 7.27-7.16 (m, 3H), 7.02 (t, J=8.4 Hz, 1H), 6.52 (s, 1H), 3.95-3.88 (m, 1H), 3.84-3.79 (m, 1H), 3.50-3.41 (m, 1H), 3.29-3.23 (m, 1H), 3.06-2.95 (m, 1H), 2.94-2.90 (m, 4H), 2.89-2.75 (m, 4H), 2.71 (dd, J=13.6 Hz, 1H), 1.95-1.88 (m, 1H), 1.85-1.68 (m, 3H), 1.55-1.43 (m, 1H).

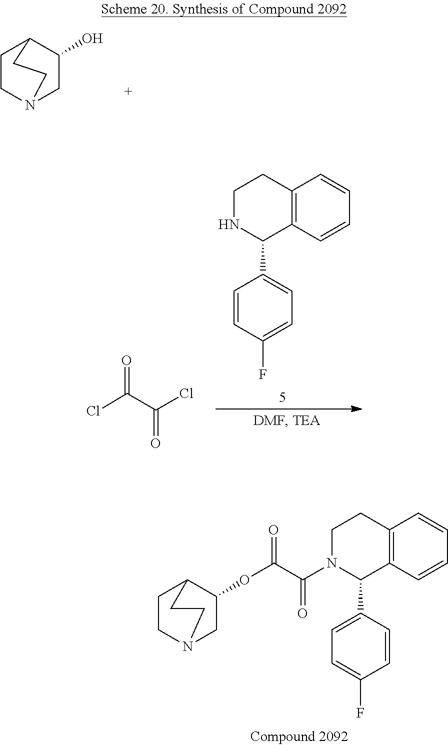 [SMILES: O[C@@H]1CN2CCC1CC2 ; O=C(Cl)C(=O)Cl ; Fc1ccc([C@@H]2NCCc3ccccc32)cc1 ; O=C(O[C@@H]1CN2CCC1CC2)C(=O)N1CCc2ccccc2[C@@H]1c1ccc(F)cc1]

Step 1: To a solution of (S)-1-(4-fluorophenyl)-1,2,3,4-tetrahydroisoquinoline 5 (227 mg, 10 mmol) in DCM (20 mL) was added the oxalyl chloride (140 mg, 11 mmol) at 0° C. Triethylamine (300 mg, 30 mmol) was added to the reaction mixture at 0° C. The mixture was stirred for 2 hours at room temperature. The mixture was filtered to remove triethylamine hydrochloride salts, then the residue was evaporated to (S)-2-(1-(4-fluorophenyl)-3,4-dihydroisoquinolin-2(1H)-yl)-2-oxoacetyl chloride as a yellow solid.

Step 2: To a solution of (S)-2-(1-(4-fluorophenyl)-3,4-dihydroisoquinolin-2(1H)-yl)-2-oxoacetyl chloride (317 mg, 10 mmol) in DMF (10 mL) was added (S)-quinuclidin-3-ol (127 mg, 10 mmol) at room temperature. Triethylamine (300 mg, 30 mmol) was added to the reaction mixture at 0° C. The mixture was stirred for 2 hours at room temperature and then water (20 mL) was added and the phases separated. The organic phase was washed with brine (50 mL×2) and dried over Na₂SO₄. After removal of the solvent, the residue was purified by HPLC to give compound 2092.

Compound 2092: LCMS: (M+H)⁺ 409; purity 100% (214 nm); retention time 1.83 min. by CP Method C

¹H NMR (400 MHz, CD₃OD) δ 7.29-7.04 (m, 8H), 6.69 (s, 1H), 5.22-4.97 (m, 1H), 3.76-3.65 (m, 1H), 3.51 (ddd, J=22.9, 12.5, 6.6 Hz, 1H), 3.42-3.33 (m, 1H), 3.09 (ddd, J=26.0, 13.3, 7.1 Hz, 1H), 2.99-2.65 (m, 6H), 2.14-2.07 (m, 1H), 1.93-1.82 (m, 1H), 1.77 (dd, J=13.0, 8.6 Hz, 1H), 1.72-1.63 (m, 1H), 1.51 (qd, J=16.1, 7.3 Hz, 1H).

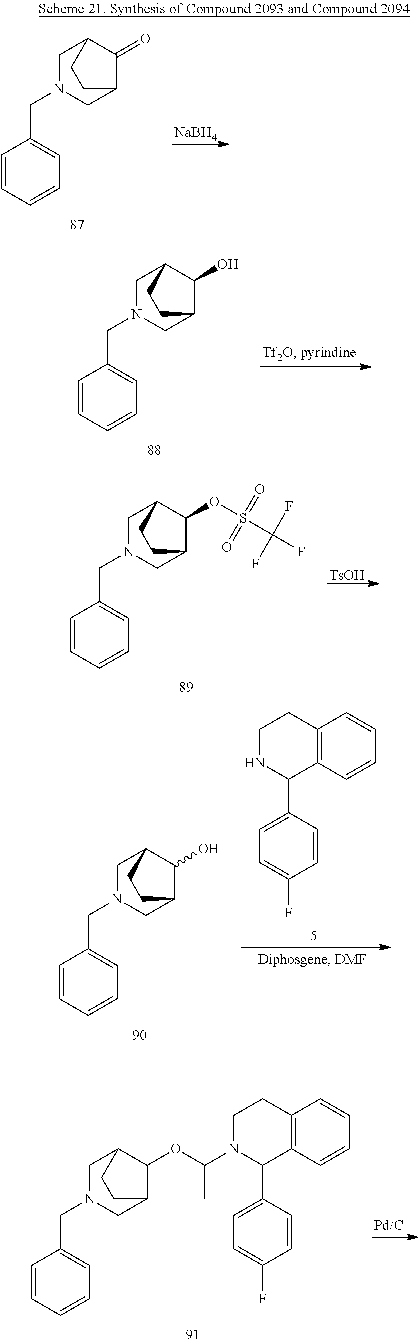

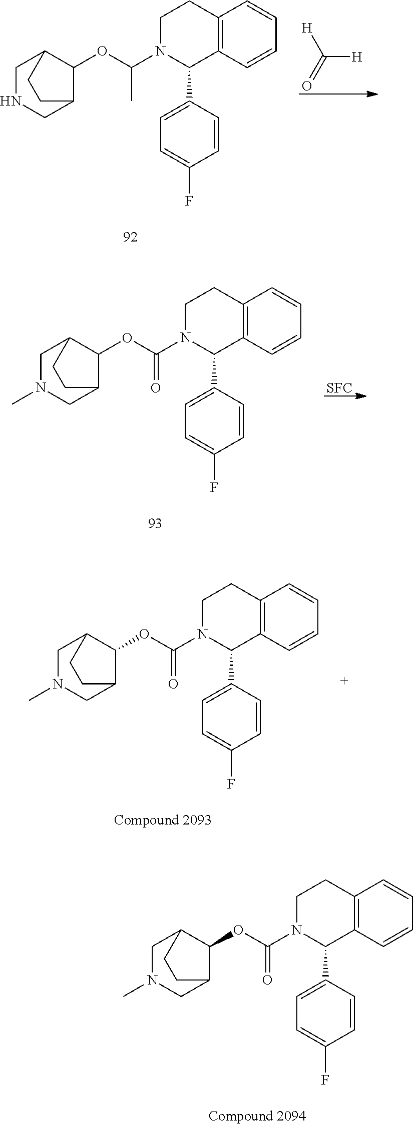

Step 13-benzyl-3-azabicyclo[3.2.1]octan-8-one 87 (4.2 g, 19.5 mmol) in methanol (40 mL) was cooled to 0° C., and then sodium borohydride (2.22 g, 58.5 mmol) was added slowly. The mixture was stirred at rt for 2 h and then the methanol was removed in vacuo. To the mixture was added water (40 mL) and it was extracted with three 50 mL portions of dichloromethane. The combined organic layers were dried with Na₂SO₄ and concentrated to give (1R,5S,8s)-3-benzyl-3-azabicyclo[3.2.1]octan-8-ol 88 (3.7 g) as a yellow oil.

LCMS: (M+H)⁺=218; purity=83% (254 nm); retention time=0.950 min. CP Method E

Step 2: A suspension of (1R,5S,8s)-3-benzyl-3-azabicyclo[3.2.1]octan-8-ol 88 (3.7 g, 17.1 mmol) and pyridine (13.5 g, 171 mmol) in dichloromethane (40 mL) was cooled to 0° C., and then trifluoromethanesulfonic anhydride (9.64 g, 34.2 mmol) was added dropwise. The mixture was stirred at rt for 1 h and diluted with 40 mL of water. The mixture was extracted with three 50 mL portions of dichloromethane. The combined organic layers were dried over Na₂SO₄ and concentrated to give trifluoromethanesulfonate 89 (5.5 g) as a yellow oil.

Step 3 To a solution of trifluoromethanesulfonate 89 (5.5 g, 15.9 mmol) in toluene (50 mL) was added p-toluenesulfonic acid (4.11 g, 23.9 mmol), 5 mL of water and 20 mL of DMSO. The mixture was stirred at reflux for 3 days and then the toluene was removed in vacuo. The mixture was diluted with water (40 mL) and extracted with three 50 mL portions of dichloromethane/methanol (20/1) and dried over Na₂SO₄. The crude product was purified by column chromatography eluting with PE/EA (2:1) to give 1.2 g of alcohols 90 as a crude yellow oil.

LCMS: (M+H)⁺=218; purity=32% (214 nm); retention time=1.207 min. CP Method A

Step 4: To a solution of alcohols 90 (654 mg, 3 mmol, 1 eq) in MeCN (15 mL) was added trichloromethyl carbonochloridate (594 mg, 3 mmol, 1 eq) and the mixture was stirred at room temperature for 2 h. The mixture was concentrated to give a white solid. The white solid was dissolved in 10 mL of DMF and (S)-1-(4-fluorophenyl)-1,2,3,4-tetrahydroisoquinoline (681 mg, 3 mmol), TEA (909 mg, 9 mmol, 3 eq) was added. The mixture was stirred at 60° C. overnight. The mixture was cooled to 25° C. and water (40 mL) was added. The mixture was extracted with three 40 mL portions of ethyl acetate. The combined organic layers were washed with brine, dried and concentrated in vacuo to give a crude product. The crude product was purified by prep-HPLC to give 940 mg of 3-benzyl-3-azabicyclo[3.2.1]octan-8-yl (1 S)-1-(4-fluorophenyl)-3,4-dihydroisoquinoline-2(1H)-carboxylate 91 as a yellow oil.

LCMS: (M+H)⁺=471; purity=46% (214 nm); retention time=1.755 min. CP Method E

Step 5: A suspension of 3-benzyl-3-azabicyclo[3.2.1]octan-8-yl (1S)-1-(4-fluorophenyl)-3,4-dihydroisoquinoline-2(1H)-carboxylate 91 (940 mg, 2 mmol) and Pd/C (10%, 100 mg) in MeOH (10 mL) were shaken under an atmosphere of hydrogen. After f the reaction was complete, the mixture was filtered through a pad of celite, and the solvent was evaporated under reduced pressure to give 390 mg of (1S)-3-aza-bicyclo[3.2.1]octan-8-yl 1-(4-fluorophenyl)-3,4-dihydroisoquinoline-2(1H)-carboxylate 92 as a yellow oil.

LCMS: (M+H)⁺=381; purity=54% (214 nm); retention time=1.835 min. CP Method C

Step 6: To a solution of (1S)-3-aza-bicyclo[3.2.1]octan-8-yl 1-(4-fluorophenyl)-3,4-dihydroisoquinoline-2(1H)-carboxylate 92 (380 mg, 1 mmol) in MeOH (5 mL) was added HCHO (0.1 mL, 1.2 mmol, 36% in water) and NaBH₃CN (189 mg, 3 mmol). The mixture was stirred at room temperature for 2 h. The mixture was diluted with water (20 mL) and extracted with three 20 mL portions of dichloromethane. The combined organic layers were dried and concentrated in vacuo to give a crude product. The crude product was purified by HPLC CP Method (Mobile Phase: A:H₂O (10 mM NH₄HCO₃) B:MeCN Gradient: 5%-95% B in 1.2 min, Flow Rate: 2.0 mL/min, Column: XBridge C18 50*4.6 mm, 3.5 μmi Oven Temperature: 40° C. UV214, MASS:100-1000) to give 100 mg of (1S)-3-methyl-3-aza-bicyclo[3.2.1]octan-8-yl 1-(4-fluorophenyl)-3,4-dihydroisoquinoline-2(1H)-carboxylate 93 as a white solid.

LCMS: (M+H)⁺=395; purity=100% (214 nm); retention time=1.600 min. CP Method E

The diastereomers were separated by chiral SFC eluting with CO₂/MeOH containing 0.2% methanolic ammonia over an EnantioPak® IG column (4.6*100 mm 5 μm) to give Compound 2093 (retention time=1.79 min) and Compound 2094 (retention time=2.73 min). Stereochemical assignment of (S) at the 1 position of the tetrahydroisoquinoline is assigned based on chiral starting material.

Compound 2093: LCMS: (M+H)⁺=395 retention time=2.170 min. CP Method C

¹H NMR (400 MHz, CDCl₃) δ 7.24-7.12 (m, 5H), 7.03 (s, 1H), 6.96 (t, J=8.8 Hz, 2H), 6.48-6.08 (m, 1H), 4.71 (s, 1H), 4.22-3.81 (m, 1H), 3.28-3.15 (m, 1H), 3.09-2.84 (m, 1H), 2.81-2.71 (m, 3H), 2.32-2.26 (m, 1H), 2.25 (s, 3H), 2.23-2.17 (m, 3H), 1.85-1.76 (m, 4H).

Compound 2094: LCMS: (M+H)⁺=395; retention time=2.159 min. CP Method C

¹H NMR (400 MHz, CDCl₃) δ 7.24-7.12 (m, 5H), 7.02 (s, 1H), 6.96 (t, J=8.8 Hz, 2H), 6.48-6.08 (m, 1H), 4.70 (s, 1H), 4.22-3.85 (m, 1H), 3.28-3.15 (m, 1H), 3.09-2.84 (m, 1H), 2.81-2.65 (m, 3H), 2.32-2.26 (m, 1H), 2.23 (s, 3H), 2.23-2.14 (m, 3H), 1.85-1.75 (m, 4H).

Compound 92 was also resolved into its diastereomers:

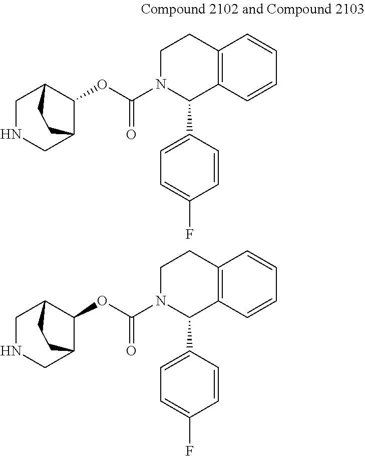

The diastereomers were separated by chiral SFC eluting with CO₂/MeOH containing 0.2% methanol/ammonia over an EnantioPak® IG column (4.6×100 mm 5 μm) to give Compound 2102 (retention time 1.27 min) and Compound 2103 (retention time 3.67 min). Stereochemical assignment of (S) at the 1 position of the tetrahydroisoquinoline is absolute based on starting materials, stereochemical assignment at is assigned based on chromatographic elution order.

Compound 2102: LCMS: (M+H)⁺ 381; purity 100% (214 nm); retention time 1.814 min. by CP Method E

¹H NMR (400 MHz, CD₃OD) δ 7.28-7.20 (m, 5H), 7.11-6.99 (m, 3H), 6.44-6.08 (m, 1H), 4.81 (s, 1H), 4.05-3.85 (m, 1H), 3.02-2.88 (m, 1H), 2.86-2.74 (m, 6H), 2.26 (s, 2H), 1.88 (s, 2H), 1.74-1.62 (m, 2H).

Compound 2103: LCMS: (M+H)⁺ 381; purity 100% (214 nm); retention time 1.817 min. by CP Method E

¹H NMR (400 MHz, CD₃OD) δ 7.28-7.21 (m, 5H), 7.11-6.98 (m, 3H), 6.44-6.08 (m, 1H), 4.80 (s, 1H), 4.05-3.85 (m, 1H), 2.99-2.88 (m, 1H), 2.86-2.76 (m, 6H), 2.26 (s, 2H), 1.88 (s, 2H), 1.74-1.63 (m, 2H).

The following compounds were prepared using General Procedure GP-3:

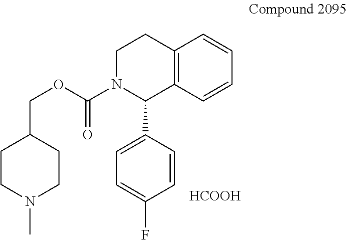

Compound 2095: LCMS: (M+H)⁺ 383.3; purity 100% (214 nm); retention time 1.569 min. by CP Method A1

¹H NMR (400 MHz, CDCl₃) δ 7.26-7.16 (m, 5H), 7.03 (d, J=7.6 Hz 1H), 6.98-6.93 (m, 2H), 6.40-6.22 (m, 1H), 4.11-4.03 (m, 3H), 3.28-3.21 (m, 3H), 2.97-2.96 (m, 1H), 2.80-2.74 (m, 1H), 2.54 (s, 3H), 2.40-2.35 (m, 2H), 1.82-1.70 (m, 5H).

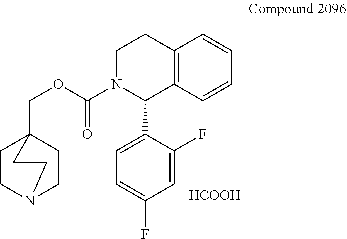

Compound 2096: LCMS: (M+H)⁺ 413.1; purity 99.76% (214 nm); retention time 1.575 min. by CP Method E

¹H NMR (400 MHz, CDCl₃) δ 7.26-7.16 (m, 3H), 7.01-6.95 (m, 2H), 6.85-6.75 (m, 2H), 6.50-6.42 (m, 1H), 4.17-3.87 (m, 3H), 3.40-3.34 (m, 1H), 3.23 (t, J=8 Hz, 6H), 3.04 (s, 1H), 2.90-2.84 (m, 1H), 1.73 (s, 6H).

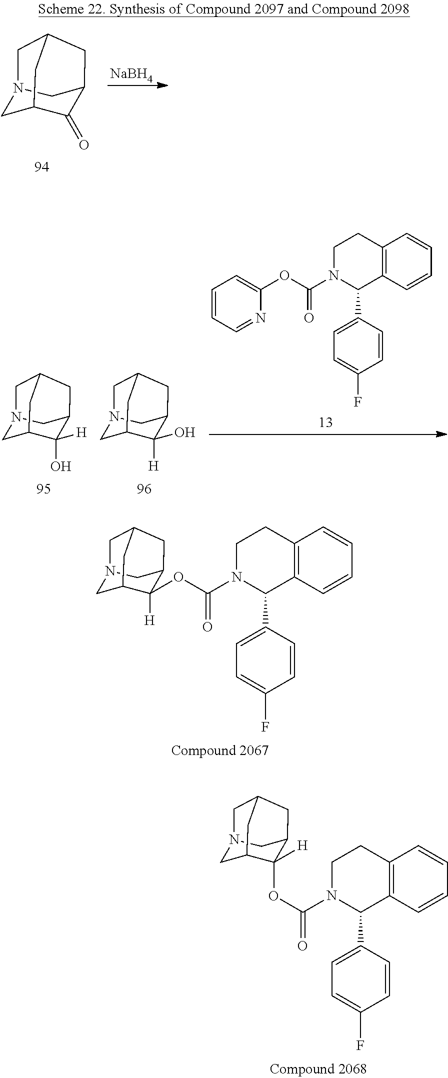

Step 1: To a solution of (1 r,3R,5S,7s)-1-azaadamantan-4-one (500 mg, 3.25 mmol) in MeOH (10 mL) cooled to 0° C., was added NaBH₄ (369 mg, 9.76 mmol). The mixture was stirred at rt for 2 hours, diluted with water (15 mL) and then extracted with three 10 mL portions of DCM. The organic phase was dried over Na₂SO₄, and then concentrated in vacuo to give the crude product as a mixture of (1 r,3R,4r,5S,7s)-1-azaadamantan-4-ol and (1 r,3R,4s,5S,7s)-1-azaadamantan-4-ol 95, 96.

LCMS: (M+H)⁺ 154.2 (214 nm); retention time 1.28 min. by CP Method B

Step 2: To a solution of a mixture of (1r,3R,4r,5S,7s)-1-azaadamantan-4-ol and (1r,3R,4s,5S,7s)-1-azaadamantan-4-ol 95, 96 (400 mg, 2.6 mmol) in DMF (10 mL) was added NaH (125 mg, 5.2 mmol) and the mixture was stirred at 0° C. for 0.5 h. Pyridin-2-yl (S)-1-(4-fluorophenyl)-3,4-dihydroisoquinoline-2(1H)-carboxylate 13 (905 mg, 2.6 mmol) was then added under N₂ at 60° C. The mixture was stirred overnight to give the desired products, which was purified by prep-HPLC to give the isomers. LCMS: (M+H)⁺=407.1; 407.2 (254 nm); retention time=1.544; 1.595 min. CP Method E

Compound 2097: LCMS: (M+H)⁺=407.2; purity=100% (214 nm); retention time=1.444 min. CP Method A1

¹H NMR (400 MHz, CDCl₃) δ 7.26-7.15 (m, 5H), 7.05-6.97 (m, 3H), 6.37-6.11 (t, 1H), 5.07 (s, 1H), 4.08-3.94 (m, 1H), 3.66-3.63 (m, 2H), 3.42 (s, 3H), 3.32 (d, J=12.4 Hz, 2H), 3.00-2.95 (m, 1H), 2.87-2.82 (m, 1H), 2.37 (s, 2H), 2.23 (s, 1H), 2.07 (q, J=14 Hz, 4H).

Compound 2098: LCMS: (M+H)⁺=407.2; purity=100% (214 nm); retention time=1.614 min. CP Method A1

¹H NMR (400 MHz, CDCl₃) δ 7.24-7.16 (m, 5H), 7.06 (d, J=7.6 Hz, 1H), 7.00-6.96 (m, 2H), 6.41-6.18 (m, 1H), 5.08 (s, 1H), 4.07-4.02 (m, 1H), 3.54-3.35 (m, 7H), 2.99-2.97 (m, 1H), 2.86-2.81 (m, 1H), 2.42 (s, 2H), 2.20 (s, 2H), 1.87-1.85 (m, 3H).

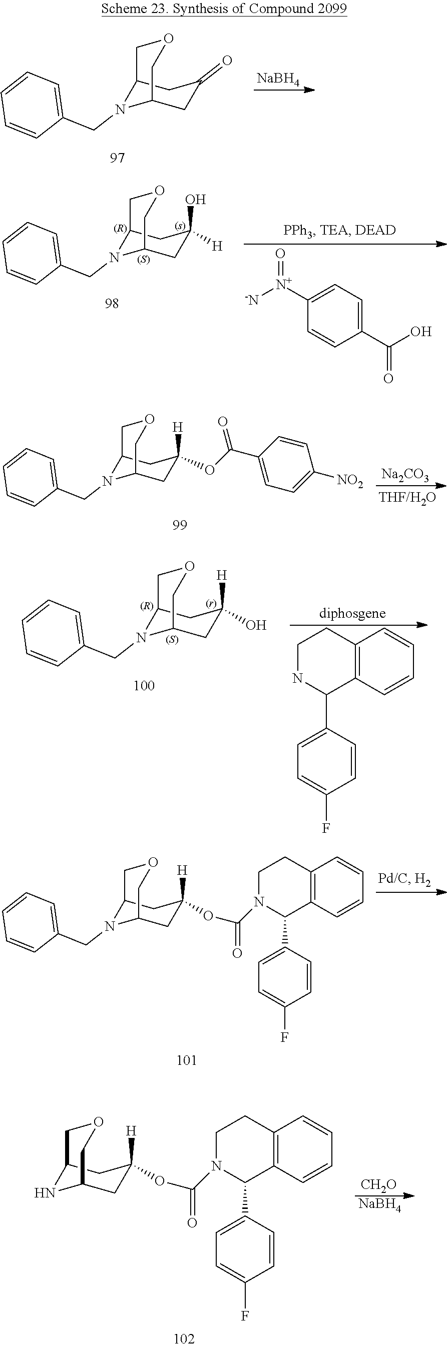

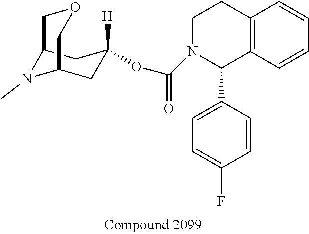

Step 1: To a solution of 9-benzyl-3-oxa-9-azabicyclo[3.3.1]nonan-7-one 97 (0.81 g, 3.5 mmol) in MeOH (10 mL) was added NaBH₄ (0.53 g, 14 mmol) at 0° C. The reaction was stirred at rt for 16 h and then concentrated. The residue was dissolved in water (15 mL) and extracted with three 30 mL portions of DCM. The combined organic phase was dried over Na₂SO₄ and concentrated to give (1R,5S,7s)-9-benzyl-3-oxa-9-azabicyclo[3.3.1]nonan-7-ol 98 (0.82 g, 3.5 mmol) as a light brown solid.

LCMS: (M+1)+234; retention time 1.109 min. by CP Method B

Step 2 To a solution of (1R,5S,7s)-9-benzyl-3-oxa-9-azabicyclo[3.3.1]nonan-7-ol 98 (0.82 g, 3.5 mmol), 4-nitrobenzoic acid (0.88 g, 5.27 mmol) and PPh₃ (1.38 g, 5.27 mmol) in dry THF (20 mL) at 0° C. was added, dropwise, DEAD (1.1 mL, 7.02 mmol). The reaction was stirred at 25° C. for 16 h and then concentrated. The residue was dissolved in MeOH (10 mL) and the resulting precipitate collected by filtration and dried to afford 9-benzyl-3-oxa-9-azabicyclo[3.3.1]nonan-7-yl 4-nitrobenzoate 99 (0.4 g, 1.04 mmol) as a white solid.

LCMS: (M+1)⁺ 383; Retention time 1.68 min. by CP Method B

Step 3: To a solution of methyl 9-benzyl-3-oxa-9-azabicyclo[3.3.1]nonan-7-yl 4-nitrobenzoate 99 (0.45 g, 1.2 mmol) in MeOH (10 mL) and H₂O (5 mL) was added Na₂CO₃ (0.25 g, 2.35 mmol) at rt. The reaction mixture was stirred at 25° C. for 3 days and then extracted with three 10 mL portions of DCM. The combined organic phase was dried and concentrated. The crude residue was purified by prep-HPLC to give (1R,5S,7r)-9-benzyl-3-oxa-9-azabicyclo[3.3.1]nonan-7-ol 100 (0.15 g, 0.64 mmol) as a white solid.

LCMS: (M+1)⁺ 234; retention time 1.434 min. by CP Method C

Step 4: To a solution (1R,5S,7r)-9-benzyl-3-oxa-9-azabicyclo[3.3.1]nonan-7-ol 100 (0.15 g, 0.64 mmol) in dry CH₃CN (5 mL) was added diphosgene (0.12 g, 0.96 mmol) at rt. The reaction mixture was stirred at ambient temperature for 2 h. and then concentrated in vacuo. The residue was dissolved in dry DMF (10 mL) and (S)-1-(4-fluorophenyl)-1,2,3,4-tetrahydroisoquinoline (145 mg, 0.64 mmol) and TEA (0.18 mL, 1.28 mmol) were added. The mixture was heated to 70° C. for 16 h, cooled to rt and purified by prep-HPLC to give (1S,1′R,5'S,7′r)-)-9-benzyl-3-oxa-9-azabicyclo[3.3.1]nonan-7-yl-1-(4-fluorophenyl)-3,4-dihydroisoquinoline-2(1H)-carboxylate 101 (0.15 g, 0.31 mmol) as a white solid

LCMS: (M+1)+487; retention time 1.927 min. by CP Method B

Step 5

To a solution (1S,1′R,5'S,7′r)-)-9-benzyl-3-oxa-9-azabicyclo[3.3.1]nonan-7-yl-1-(4-fluorophenyl)-3,4-dihydroisoquinoline-2(1H)-carboxylate 101 (0.15 g, 0.31 mmol) in MeOH (5 mL) was added 10% Pd/C (50 mg). The system was evacuated and then backfilled with hydrogen. After reaction mixture was stirred at rt for 16 h., it was filtered and the filtrate concentrated to give (1S,1′R,5'S,7′r)-3-oxa-9-azabicyclo[3.3.1]nonan-7-yl-1-(4-fluorophenyl)-3,4-dihydroisoquinoline-2(1H)-carboxylate 102 (0.117 g, 0.3 mmol) as a light brown solid.

LCMS: (M+1)+397; Retention time 1.76 min. by CP Method B

Step 6

To a solution of (1S,1′R,5'S,7′r)-3-oxa-9-azabicyclo[3.3.1]nonan-7-yl-1-(4-fluorophenyl)-3,4-dihydroisoquinoline-2(1H)-carboxylate 102 (117 mg, 0.3 mmol) in MeOH (4 mL) was added 38% aqueous formaldehyde (71.8 mg, 0.9 mmol). The reaction mixture was stirred at 60° C. for 16 h. and then cooled to 0° C. A single portion of NaBH₃CN (37.7 mg, 0.6 mmol) was added and the mixture was stirred at rt for 2 h. The crude reaction mixture was purified by prep-HPLC to give Compound 2099(.

Compound 2099: LCMS: (M+1)+411; retention time=1.902 min. by CP Method C

¹H NMR (400 Hz, DMSO-d₆): 7.25-7.13 (m, 9H), 6.25 (s, 1H), 5.58-5.53 (m, 1H), 3.91 (br, 1H), 3.70-3.60 (m, 4H), 3.30-3.23 (m, 1H), 2.91-2.82 (m, 2H), 2.70 (s, 2H), 2.40 (s, 3H), 1.91-1.78 (m, 4H).

The following compounds were prepared using General Procedure GP-4:

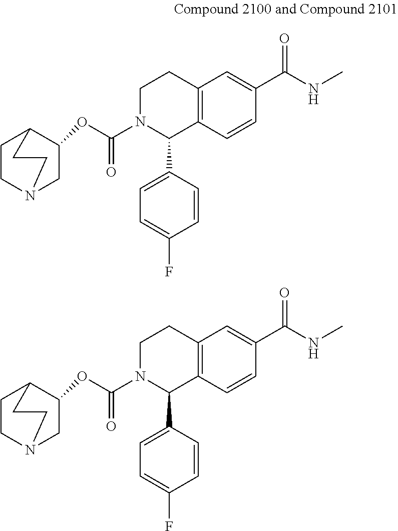 [SMILES: CNC(=O)c1ccc2c(c1)CCN(C(=O)O[C@@H]1CN3CCC1CC3)[C@H]2c1ccc(F)cc1 ; CNC(=O)c1ccc2c(c1)CCN(C(=O)O[C@@H]1CN3CCC1CC3)[C@@H]2c1ccc(F)cc1]

The diastereomers were separated by chiral SFC eluting with CO₂/MeOH containing 0.2% methanol/ammonia over an EnantioPak® IG column (4.6×100 mm 5 μm) to give Compound 2100 (retention time 2.68 min) and Compound 2101 (retention time 3.2 min). Stereochemical assignment of (S) at quinuclidine is absolute based on starting materials; stereochemical assignment at the 1 position of the tetrahydroisoquinoline is based on chromatographic elution order as compared to diastereomers of related analogues of known configuration.

Compound 2100: LCMS: (M+H)⁺ 438; retention time 1.464 min. by CP Method C

¹H NMR (400 MHz, CDCl₃) δ 7.66 (s, 1H), 7.55-7.48 (m, 1H), 7.20-7.13 (m, 2H), 7.10 (d, J=8.0 Hz, 1H), 6.97 (t, J=8.8 Hz, 2H), 6.52-6.21 (m, 1H), 6.10 (d, J=4.4 Hz, 1H), 4.86-4.76 (m, 1H), 4.22-3.98 (m, 1H), 3.32-3.18 (m, 2H), 3.03 (d, J=4.8 Hz, 4H), 2.91-2.84 (m, 2H), 2.83-2.75 (m, 2H), 2.74-2.67 (m, 2H), 2.08-2.01 (m, 1H), 1.86-1.76 (m, 1H), 1.74-1.64 (m, 1H), 1.58-1.51 (m, 1H), 1.48-1.35 (m, 1H).

Compound 2101: LCMS: (M+H)⁺ 438; purity 100% (214 nm); retention time 1.195 min. by CP Method E

¹H NMR (400 MHz, CDCl₃) δ 7.66 (s, 1H), 7.51 (d, J=7.2 Hz, 1H), 7.20-7.13 (m, 2H), 7.09 (d, J=8.0 Hz, 1H), 6.97 (t, J=8.4 Hz, 2H), 6.52-6.21 (m, 1H), 6.10 (d, J=4.0 Hz, 1H), 4.86-4.76 (m, 1H), 4.22-3.98 (m, 1H), 3.32-3.18 (m, 2H), 3.03 (d, J=4.8 Hz, 4H), 2.91-2.66 (m, 6H), 2.11-2.01 (m, 1H), 1.86-1.62 (m, 2H), 1.60-1.52 (m, 1H), 1.47-1.35 (m, 1H).

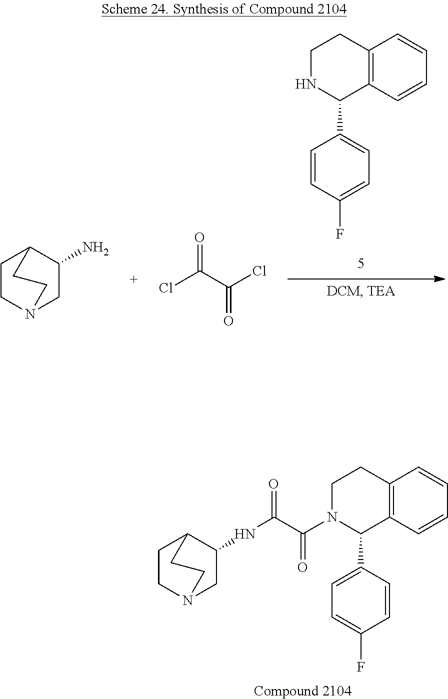 [SMILES: Fc1ccc([C@@H]2NCCc3ccccc32)cc1 ; N[C@@H]1CN2CCC1CC2 ; O=C(Cl)C(=O)Cl ; "DCM, TEA" ; O=C(N[C@@H]1CN2CCC1CC2)C(=O)N1CCc2ccccc2[C@@H]1c1ccc(F)cc1 ; "Compound 2104"]

Step 1 To a solution of (S)-1-(4-fluorophenyl)-1,2,3,4-tetrahydroisoquinoline 5 (227 mg, 10 mmol) in dichloromethane (20 mL) at 0° C. was added oxalyl chloride (140 mg, 11 mmol) followed by triethylamine (300 mg, 30 mmol). The mixture was stirred for 2 hours at room temperature. The mixture was evaporated to (S)-2-(1-(4-fluorophenyl)-3,4-dihydroisoquinolin-2(1H)-yl)-2-oxoacetyl chloride as a yellow solid.

Step 2: To a solution of (S)-2-(1-(4-fluorophenyl)-3,4-dihydroisoquinolin-2(1H)-yl)-2-oxoacetyl chloride (317 mg, 10 mmol) in DMF (10 mL) was added (S)-quinuclidin-3-amine (127 mg, 10 mmol) at room temperature. After cooling the reaction mixture to 0° C., triethylamine (300 mg, 30 mmol) was added the mixture was stirred for 2 hours at room temperature. Water (20 mL) was added and the phases were separated. The organic phase was washed with brine (2×50 mL) and dried over Na₂SO₄. The solvent was removed and the residue purified by HPLC to give compound 2104.

Compound 2104: LCMS: (M+H)⁺ 408; purity 100% (214 nm); retention time 1.51 min. by CP Method C

¹H NMR (400 MHz, DMSO-d₆) δ 8.91 (d, J=6.6 Hz, 1H), 7.36-7.06 (m, 8H), 6.61 (s, 1H), 3.83 (s, 1H), 3.68-3.58 (m, 1H), 3.44 (d, J=44.2 Hz, 1H), 3.14-2.98 (m, 2H), 2.87-2.59 (m, 5H), 2.43-2.03 (dd, J=13.0, 8.6 Hz, 1H), 1.76 (t, J=26.7 Hz, 2H), 1.55 (d, J=6.2 Hz, 2H), 1.26 (d, J=23.1 Hz, 1H).

The following compound was prepared using General Procedure GP-3:

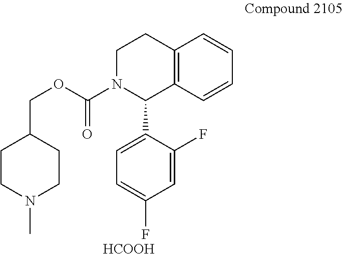 [SMILES: CN1CCC(COC(=O)N2CCc3ccccc3[C@@H]2c2ccc(F)cc2F)CC1 ; "HCOOH"]

Compound 2105: LCMS: (M+H)⁺ 401.2; purity 100% (214 nm); retention time 1.583 min. by CP Method A1

¹H NMR (400 MHz, CDCl₃) δ 7.22-7.13 (m, 3H), 6.98 (d, J=7.2 Hz, 2H), 6.84-6.73 (m, 2H), 6.44 (s, 1H), 4.19-3.97 (m, 3H), 3.37-3.35 (m, 3H), 3.00 (s, 1H), 2.88-2.82 (m, 1H), 2.60 (s, 3H), 2.48-2.43 (m, 2H), 1.79-1.72 (m, 5H).

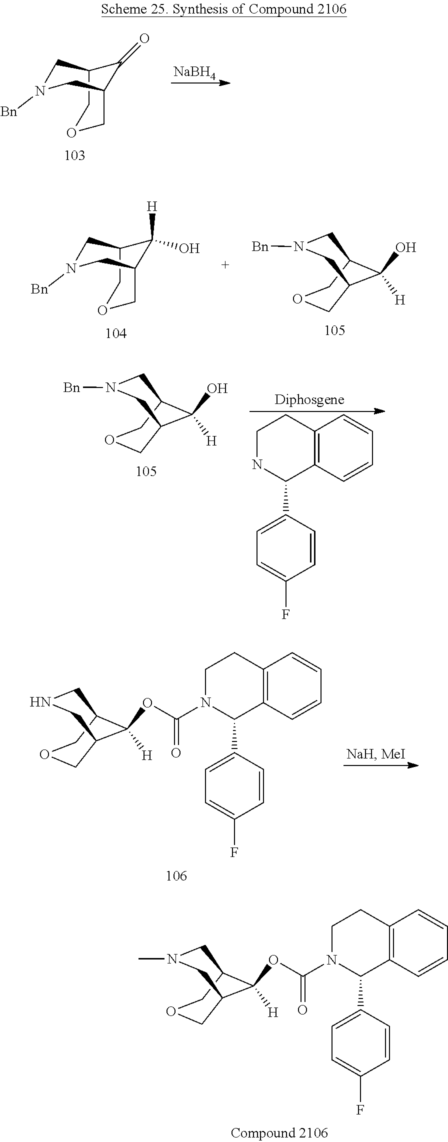

Step 1: To a solution of (1R, 5S)-7-benzyl-3-oxa-7-azabicyclo [3.3.1] nonan-9-one 103 (1.5 g, 6.49 mmol) in MeOH (30 ml) was added NaBH₄ (741 mg, 19.5 mmol) slowly at 0° C. The reaction mixture was stirred at 0° C. for 2 hr and then concentrated. After dilution with water (30 mL) and extraction with three 30 mL portions of EA, the combined organic phase was washed by brine (60 mL), dried and concentrated to obtain a crude solid. Purification by prep-HPLC afforded (1R,5S,9s)-7-benzyl-3-oxa-7-azabicyclo[3.3.1]nonan-9-ol 105 (450 mg).

LCMS: (M+H)⁺ 233, purity: 100% (214 nm) retention time 1.24 min, by CP Method C

Step 2: To a solution of (1R, 5S, 9s)-7-benzyl-3-oxa-7-azabicyclo [3.3.1] nonan-9-ol 105 (233 mg, 1 mmol) dissolved in ACN (5 mL) was added diphosgene (119 mg, 0.6 mmol) at 0° C. The resulting mixture was stirred at 25° C. for 1 hour and then concentrated to obtain a light yellow solid. This material was added into a solution of (S)-1-(4-fluorophenyl)-1, 2, 3, 4-tetrahydroisoquinoline (227 mg, 1 mmol) and TEA (303 mg, 3 mmol) in DMF (10 mL). After the reaction mixture was stirred at 60° C. for 16 hours, it was poured into water (50 mL) and extracted with three 20 mL portions of EA. The combined organic phase was washed by with three 50 mL portions of brine, dried over anhydrous Na₂SO₄ and concentrated to obtain a crude solid, which was purified by prep-HPLC to afford (S)-((1R,5S,9s)-3-oxa-7-azabicyclo[3.3.1]nonan-9-yl)-1-(4-fluorophenyl)-3,4-dihydroisoquinoline-2(1H)-carboxylate 106 (25 mg).

LCMS: (M+H)⁺ 397, purity: 100% (214 nm) retention time 1.56 min by CP Method C

Step 3: To a solution of (S)-((1R, 5S, 9s)-3-oxa-7-azabicyclo [3.3.1] nonan-9-yl)-1-(4-fluorophenyl)-3, 4-dihydroisoquinoline-2(1H)-carboxylate (25 mg, 0.06 mmol) in DMF (2 mL) was added NaH (4 mg, 0.1 mmol) at 0° C. After the addition, Mel (14 mg, 0.1 mmol) was introduced and the mixture was stirred at rt for 0.5 h. The reaction mixture was diluted with water (20 mL) and extracted with three 10 mL portions of EA. The combined organic phase was washed with brine (20 mL), dried over anhydrous Na₂SO₄, filtered and concentrated to obtain a crude oil, which was purified by prep-HPLC to afford (1R,5S,9s)-7-methyl-3-oxa-7-azabicyclo[3.3.1]nonan-9-yl (S)-1-(4-fluorophenyl)-3,4-dihydroisoquinoline-2(1H)-carboxylate compound 2106.

Compound 2106: LCMS: (M+H)⁺ 411, purity 100% (214 nm), retention time=1.858 min. by CP Method C

¹H NMR (400 MHz, CDCl₃) δ 7.24-7.08 (m, 5H), 7.02-6.89 (m, 3H), 6.16 (s, 1H), 4.23 (d, J=13.4 Hz, 1H), 4.12-4.00 (m, 2H), 3.88 (t, J=12.6 Hz, 2H), 3.68-3.59 (m, 2H), 3.44 (t, J=3.2 Hz, 1H), 3.39 (s, 3H), 3.32-3.20 (m, 2H), 3.06 (d, J=13.3 Hz, 1H), 3.00 (dd, J=13.7, 7.7 Hz, 1H), 2.85-2.76 (m, 1H), 1.78 (s, 2H), 1.59 (s, 2H).

The following compounds were prepared using General Procedure GP-4:

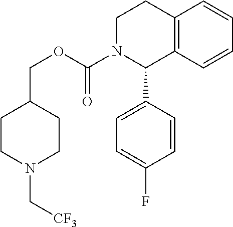 [SMILES: O=C(OCC1CCN(CC(F)(F)F)CC1)N1CCc2ccccc2[C@@H]1c1ccc(F)cc1]

Compound 2107: LCMS: (M+H)⁺ 451.2; purity 100% (214 nm); retention time 1.619 min. by CP Method E

¹H NMR (400 MHz, CDCl₃) δ 7.25-7.18 (m, 5H), 7.05 (d, J=7.6 Hz, 1H), 6.98 (t, J=8.8 Hz, 2H), 6.43-6.26 (m, 1H), 4.08-3.97 (m, 3H), 3.29-3.22 (m, 1H), 3.02-2.95 (m, 5H), 2.82-2.76 (m, 1H), 2.37 (t, J=11.2 Hz, 2H), 1.71 (d, J=10 Hz, 3H), 1.44-1.35 (m, 2H).

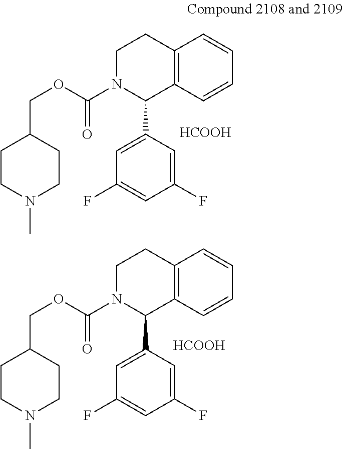 [SMILES: CN1CCC(COC(=O)N2CCc3ccccc3[C@@H]2c2cc(F)cc(F)c2)CC1 ; "HCOOH" ; CN1CCC(COC(=O)N2CCc3ccccc3[C@H]2c2cc(F)cc(F)c2)CC1 ; "HCOOH"]

The diastereomers were separated by chiral SFC eluting with CO₂/EtOH containing 1% methanol/ammonia over an EnantioPak® OD-H column (4.6×250 mm 5 μm) to give Compound 2108 (retention time 7.13 min) and Compound 2109 (retention time 5.43 min). Stereochemical assignment at the 1 position of the tetrahydroisoquinoline is based on chromatographic elution order as compared to diastereomers of related analogues of known configuration.

Compound 2108: LCMS: (M+H)⁺ 401.2; purity 98.76% (214 nm); retention time 1.455 min. by CP Method A1

¹H NMR (400 MHz, CDCl₃) δ 7.28-7.19 (m, 3H), 7.07 (d, J=7.6 Hz, 1H), 6.72-6.67 (m, 3H), 6.35-6.16 (m, 1H), 4.12-3.95 (m, 3H), 3.33-3.26 (m, 3H), 2.94 (s, 1H), 2.78-2.74 (m, 1H), 2.53 (s, 3H), 2.39-2.34 (m, 2H), 1.81-1.66 (m, 5H).

Compound 2109: LCMS: (M+H)⁺ 401.2; purity 100% (214 nm); retention time 1.455 min. by CP Method A1

¹H NMR (400 MHz, CDCl₃) δ 7.24-7.19 (m, 3H), 7.07 (d, J=7.6 Hz, 1H), 6.73-6.67 (m, 3H), 6.35-6.16 (m, 1H), 4.11-4.01 (m, 3H), 3.33-3.25 (m, 1H), 3.22-3.19 (m, 2H), 2.94 (s, 1H), 2.76 (d, J=16 Hz, 1H), 2.48 (s, 3H), 2.29 (t, J=11.2 Hz, 2H), 1.78 (d, J=10 Hz, 3H), 1.64-1.61 (m, 2H).

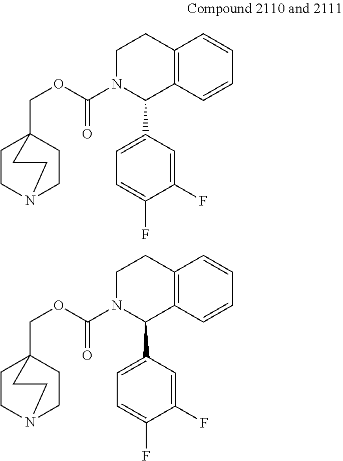

The diastereomers were separated by chiral SFC eluting with CO₂/EtOH containing 1% methanol/ammonia over an EnantioPak® IG column (4.6×250 mm 5 μm) to give Compound 2110 (retention time 1.94 min) and Compound 2111 (retention time 3.01 min). Stereochemical assignment at the 1 position of the tetrahydroisoquinoline is based on chromatographic elution order as compared to diastereomers of related analogues of known configuration.

Compound 2110: LCMS: (M+H)⁺ 413.3; purity retention time 1.461 min. by CP Method A1

¹H NMR (400 MHz, CDCl₃) δ 7.24-7.19 (m, 3H), 7.07-6.99 (m, 3H), 6.94 (s, 1H), 6.38-6.19 (m, 1H), 4.05-3.99 (m, 1H), 3.97-3.83 (m, 2H), 3.28-3.21 (m, 1H), 2.91-2.87 (m, 7H), 2.80-2.74 (m, 1H), 1.43-1.39 (m, 6H).

Compound 2111: LCMS: (M+H)⁺ 413.1; retention time 1.449 min. by CP Method A1

¹H NMR (400 MHz, CDCl₃) δ 7.26-7.19 (m, 3H), 7.07-6.99 (m, 3H), 6.94 (s, 1H), 6.39-6.18 (m, 1H), 4.06-3.97 (s, 1H), 3.85-3.83 (m, 2H), 3.28-3.21 (m, 1H), 2.91-2.87 (m, 7H), 2.80-2.74 (m, 1H), 1.43-1.39 (m, 6H).

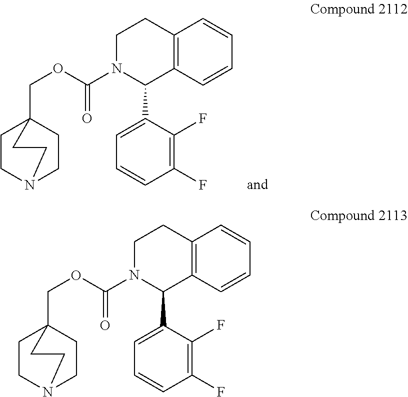

The diastereomers were separated by chiral SFC eluting with CO₂/EtOH containing 1% methanol/ammonia over an EnantioPak® AS column (4.6×100 mm 5 μm) to give Compound 2112 (retention time 2.02 min) and Compound 2113 (retention time 2.96 min). Stereochemical assignment at the 1 position of the tetrahydroisoquinoline is based on chromatographic elution order as compared to diastereomers of related analogues of known configuration.

Compound 2112: LCMS: (M+H)⁺ 413; retention time 1.704 min. by CP Method C

¹H NMR (400 MHz, CDCl₃) δ 7.24-7.13 (m, 3H), 7.12-7.03 (m, 2H), 7.01-6.93 (m, 1H), 6.86 (t, J=6.4 Hz, 1H), 6.47 (s, 1H), 4.24-3.98 (m, 1H), 3.85 (t, J=10.4 Hz, 1H), 3.80-3.66 (m, 1H), 3.61-3.42 (m, 1H), 3.08-2.95 (m, 1H), 2.94-2.76 (m, 7H), 1.47-1.28 (m, 6H).

Compound 2113: LCMS: (M+H)⁺ 413; purity 100% (214 nm); retention time 1.716 min. by CP Method C

¹H NMR (400 MHz, CDCl₃) δ 7.24-7.13 (m, 3H), 7.12-7.03 (m, 2H), 7.02-6.93 (m, 1H), 6.89-6.83 (m, 1H), 6.46 (s, 1H), 4.22-3.98 (m, 1H), 3.86 (d, J=10.4 Hz, 1H), 3.80-3.66 (m, 1H), 3.61-3.42 (m, 1H), 3.08-2.97 (m, 1H), 2.94-2.76 (m, 7H), 1.45-1.25 (m, 6H).

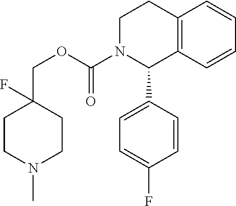 [SMILES: CN1CCC(F)(COC(=O)N2CCc3ccccc3[C@@H]2c2ccc(F)cc2)CC1]

Compound 2114: LCMS: (M+H)⁺ 401; purity 94.01% (214 nm); retention time 1.584 min. by CP Method A2

¹H NMR (400 MHz, CDCl₃) δ 7.20 (dt, J=8.5, 6.2 Hz, 5H), 7.03 (d, J=7.2 Hz, 1H), 6.96 (t, J=8.7 Hz, 2H), 6.34 (d, J=59.3 Hz, 1H), 4.17 (dd, J=19.8, 11.9 Hz, 2H), 3.26 (t, J=10.0 Hz, 1H), 2.99 (s, 2H), 2.81 (t, J=18.5 Hz, 3H), 2.45 (t, J=12.4 Hz, 2H), 2.40 (s, 3H), 2.03-1.73 (m, 4H).

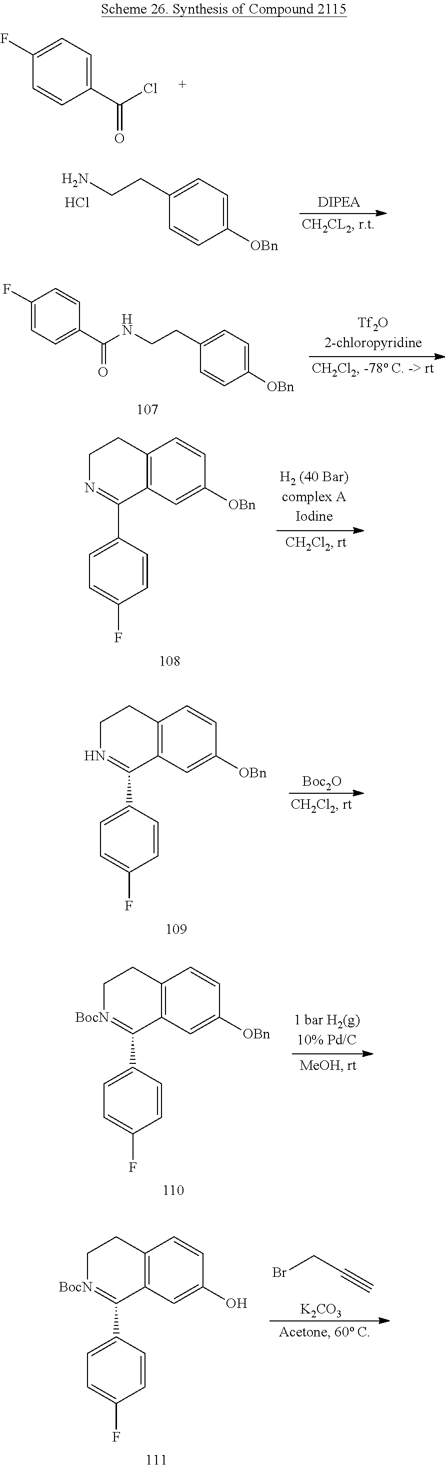

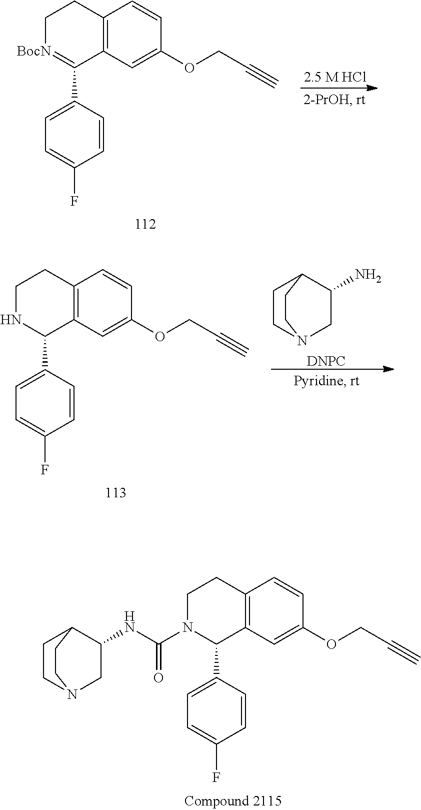

Step 1: A solution of 4-fluorobenzoyl chloride (1.716 mL, 14.51 mmol) in dichloromethane (10 mL) was added dropwise to a stirred suspension of 2-(4-benzyloxy-phenyl)-ethylamine hydrochloride (4.02 g, 15.23 mmol) and N,N-diisopropylethylamine (6.32 mL, 36.3 mmol) in dichloromethane (50 mL) and stirred for 1 hour. The reaction mixture was diluted with a mixture of saturated aqueous NH₄Cl (75 mL) and water (10 mL). The biphasic system was filtered and the filter cake rinsed with water (5 mL) and dichloromethane (5 mL). The residue was dried to give a first crop of N-(4-(benzyloxy)phenethyl)-4-fluorobenzamide 107 (3.12 g) as a white solid. The filtrate layers were separated and the aqueous phase was extracted with dichloromethane (50 mL). The combined organics were washed with brine (50 mL), dried on Na₂SO₄, and evaporated under reduced pressure to give a second crop of N-(4-(benzyloxy)phenethyl)-4-fluorobenzamide 107 (2.85 g) as a pale solid. Both crops contain residual N,N-diisopropylethylamine hydrochloric acid salt and were used as such.

LCMS: 100%, RT=2.130 min., (M+H)⁺=350 (MC Method A). ¹H NMR (400 MHz, DMSO-d6) δ 8.64 (t, J=5.6 Hz, 1H), 7.96-7.85 (m, 2H), 7.49-7.21 (m, 7H), 7.20-7.09 (m, 2H), 6.99-6.89 (m, 2H), 5.06 (s, 2H), 3.50-3.39 (m, 2H), 2.78 (t, J=7.5 Hz, 2H).

Step 2: At −78° C., triflic anhydride (3.11 mL, 18.72 mmol) was added dropwise to a stirred suspension of N-(4-(benzyloxy)phenethyl)-4-fluorobenzamide 107 (5.45 g, crude) and 2-chloropyridine (1.904 mL, 20.28 mmol) in dichloromethane (55 mL) under nitrogen atmosphere. The reaction mixture was stirred for 1 hour and then allowed to slowly warm to room temperature, overnight. An additional quantity of 2-chloropyridine (0.476 mL, 5.07 mmol) was added and the reaction mixture was cooled again to −78° C. Additional triflic anhydride (0.777 mL, 4.68 mmol) was added dropwise. After 30 minutes, the reaction mixture was slowly warmed to room temperature and stirred for an additional 2 hours. The reaction mixture was washed with aqueous NaOH (1 M, 50 mL) and the aqueous phase was extracted with dichloromethane (25 mL). The combined organics were washed with brine (30 mL), dried on Na₂SO₄, and evaporated under reduced pressure. The residue was purified by flash column chromatography (silica, 10% to 100% ethyl acetate in heptane) to yield 7-(benzyloxy)-1-(4-fluorophenyl)-3,4-dihydroisoquinoline 108 (3.96 g, 90 (w/w) % pure) as a pale solid.

LCMS: 90%, RT=1.761 min., (M+H)⁺=332 (MC Method A). ¹H NMR (400 MHz, Chloroform-d) δ 7.57-7.47 (m, 2H), 7.46-7.28 (m, 5H), 7.19 (d, J=8.3 Hz, 1H), 7.17-7.00 (m, 3H), 6.82 (d, J=2.5 Hz, 1H), 4.99 (s, 2H), 3.85-3.75 (m, 2H), 2.76-2.66 (m, 2H).

Step 3: [{lr(H)[(S,S)-(f)-binaphane]}₂(μ-I)₃]⁺I⁻ (Complex A, 0.078 g, 0.031 mmol) and iodine (0.079 g, 0.310 mmol) were added to a stirred solution of 7-(benzyloxy)-1-(4-fluorophenyl)-3,4-dihydroisoquinoline 108 (4.115 g, 12.42 mmol) in dichloromethane (100 mL). The resulting suspension was stirred in an autoclave charged with 40 bars of hydrogen gas at room temperature overnight. The reaction mixture was concentrated under reduced pressure. The residue was crystallized from a warm mixture of dichloromethane and methanol (9:1, ˜15 mL). The solids were filtered off, rinsed with a small amount of dichloromethane (2 mL), and dried under reduced pressure to give a first crop of (S)-7-(benzyloxy)-1-(4-fluorophenyl)-1,2,3,4-tetrahydroisoquinoline 109 (1.390 g) as a pale solid. The mother liquid was concentrated under reduced pressure. Crystallization of the residue from a mixture of dichloromethane and methanol (19:1, ˜10 mL) by slow evaporation of the solvent at room temperature over 24 hours gave a second crop of (S)-7-(benzyloxy)-1-(4-fluorophenyl)-1,2,3,4-tetrahydroisoquinoline 109 (0.890 g) as a brown solid.

Crop 1: LCMS: 93%, RT=1.723 min., (M+H)⁺=334 (MC Method A). ¹H NMR (400 MHz, DMSO-d6) δ 7.36-7.21 (m, 8H), 7.15-7.07 (m, 2H), 7.04 (d, J=8.4 Hz, 1H), 6.79 (dd, J=8.3, 2.7 Hz, 1H), 6.22 (d, J=2.7 Hz, 1H), 4.97-4.85 (m, 3H), 3.09-2.98 (m, 1H), 2.90-2.75 (m, 2H), 2.69-2.58 (m, 1H). Chiral SFC: 99% e.e. (MC Method G).

Step 4: Di-tert-butyl dicarbonate (2.376 g, 10.89 mmol) was added to a suspension of (S)-7-(benzyloxy)-1-(4-fluorophenyl)-1,2,3,4-tetrahydroisoquinoline 109 (3.30 g, 9.90 mmol) in dichloromethane (50 mL). After stirring for 2 hours, the reaction mixture was concentrated to a smaller volume (˜10 mL) under reduced pressure, filtered through a nylon 0.45 μm filter and then purified by flash column chromatography (silica, 3 to 50% ethyl acetate in heptane) to give tert-butyl (S)-7-(benzyloxy)-1-(4-fluorophenyl)-3,4-dihydroisoquinoline-2(1H)-carboxylate 110 (2.592 g) as a pale yellow oil.

LCMS: 99%, RT=2.431 min., (M+Na)⁺=456 (MC Method A). ¹H NMR (400 MHz, Chloroform-d) δ 7.40-7.27 (m, 5H), 7.21-7.13 (m, 2H), 7.10 (d, J=8.5 Hz, 1H), 6.99-6.90 (m, 2H), 6.87 (dd, J=8.4, 2.7 Hz, 1H), 6.62 (s, 1H), 6.48-5.97 (m, 1H), 5.04-4.90 (m, 2H), 4.30-3.69 (m, 1H), 3.12 (ddd, J=13.2, 10.9, 4.3 Hz, 1H), 2.97-2.81 (m, 1H), 2.67 (d, J=15.9 Hz, 1H), 1.49 (s, 9H).

Step 5: Palladium on activated carbon (10 wt %, 0.399 g, 0.375 mmol) was added to a stirred solution of tert-butyl (S)-7-(benzyloxy)-1-(4-fluorophenyl)-3,4-dihydroisoquinoline-2(1H)-carboxylate 110 (3.25 g, 7.50 mmol) in methanol (50 mL) under nitrogen atmosphere. Next, hydrogen gas was bubbled through the reaction mixture for 5 minutes after which the reaction mixture was stirred vigorously under hydrogen atmosphere, overnight. The reaction mixture was purged with nitrogen for 5 minutes, filtered through layer of Celite and the filter cake rinsed with methanol (2×10 mL). The combined filtrates were evaporated under reduced pressure to give tert-butyl (S)-1-(4-fluorophenyl)-7-hydroxy-3,4-dihydroisoquinoline-2(1H)-carboxylate 111 (2.377 g) as a colorless foam.

LCMS: 97%, RT=2.129 min., (M−H)-=342 (MC Method A). ¹H NMR (400 MHz, Chloroform-d) δ 7.22-7.11 (m, 2H), 7.05 (d, J=8.2 Hz, 1H), 6.99-6.81 (m, 2H), 6.73 (dd, J=8.3, 2.7 Hz, 1H), 6.65-6.43 (m, 1H), 6.42-6.01 (m, 1H), 4.26-3.75 (m, 1H), 3.11 (ddd, J=13.2, 10.9, 4.3 Hz, 1H), 2.96-2.78 (m, 1H), 2.77-2.59 (m, 1H), 1.48 (s, 9H).

Step 6: Potassium carbonate (157 mg, 1.136 mmol) was added to a stirred solution of tert-butyl (S)-1-(4-fluorophenyl)-7-hydroxy-3,4-dihydroisoquinoline-2(1H)-carboxylate 111 (195 mg, 0.568 mmol) in acetone (2.5 mL). The suspension was stirred for 5 minutes, after which a solution of propargyl bromide (80% in toluene, 0.184 mL, 1.704 mmol) was added. The reaction mixture was sealed and heated at 60° C. for 6 hours. After cooling to room temperature, the reaction mixture was partitioned between dichloromethane (15 mL) and water (7 mL). The layers were separated using a phase-separator and the organic filtrate was evaporated under reduced pressure. The residue was purified by flash column chromatography (silica, 0 to 30% ethyl acetate in heptane) to give tert-butyl (S)-1-(4-fluorophenyl)-7-(prop-2-yn-1-yloxy)-3,4-dihydroisoquinoline-2(1H)-carboxylate 112 (135.7 mg) as a colorless oil.

LCMS: 83%, RT=2.273 min., (M−tBu+2H)⁺=326 (MC Method A). ¹H NMR (400 MHz, Chloroform-d) δ 7.25-7.15 (m, 2H), 7.12 (d, J=8.4 Hz, 1H), 7.01-6.90 (m, 2H), 6.87 (dd, J=8.4, 2.7 Hz, 1H), 6.62 (s, 1H), 6.51-5.99 (m, 1H), 4.61 (d, J=2.3 Hz, 2H), 4.24-3.80 (m, 1H), 3.12 (ddd, J=13.1, 10.9, 4.2 Hz, 1H), 3.01-2.81 (m, 1H), 2.80-2.59 (m, 1H), 2.46 (t, J=2.4 Hz, 1H), 1.49 (s, 9H).

Step 7: A solution of hydrogen chloride in 2-propanol (5 M, 2.0 mL, 10.0 mmol) was added to a stirred solution of tert-butyl (S)-1-(4-fluorophenyl)-7-(prop-2-yn-1-yloxy)-3,4-dihydroisoquinoline-2(1H)-carboxylate 112 (135 mg, 0.354 mmol) in 2-propanol (2.0 mL). The reaction mixture was stirred for 16 hours and concentrated to dryness under reduced pressure. The residue was partitioned between dichloromethane (6 mL) and aqueous NaOH (0.5 M, 4 mL). The layers were separated through a phase-separator. The organic filtrate was evaporated under reduced pressure to give (S)-1-(4-fluorophenyl)-7-(prop-2-yn-1-yloxy)-1,2,3,4-tetrahydroisoquinoline 113 (67.3 mg) as a white solid.

LCMS: 98%, RT=1.592 min., (M+H)⁺=282 (MC Method A). ¹H NMR (400 MHz, Chloroform-d) δ 7.29-7.20 (m, 2H), 7.08 (d, J=8.3 Hz, 1H), 7.04-6.95 (m, 2H), 6.80 (dd, J=8.4, 2.7 Hz, 1H), 6.33 (d, J=2.7 Hz, 1H), 5.04 (s, 1H), 4.52 (d, J=2.4 Hz, 2H), 3.29-3.19 (m, 1H), 3.06 (ddd, J=11.9, 9.1, 4.4 Hz, 1H), 3.01-2.90 (m, 1H), 2.76 (dt, J=15.9, 4.4 Hz, 1H), 2.41 (t, J=2.4 Hz, 1H).

Step 8: A solution of (S)-quinuclidin-3-amine (14.80 mg, 0.117 mmol) in pyridine (0.25 mL) was added dropwise to a stirred solution of bis(p-nitrophenyl) carbonate (35.7 mg, 0.117 mmol) in pyridine (0.50 mL) under nitrogen atmosphere. The reaction mixture was stirred for 2.5 hours, after which a solution of (S)-1-(4-fluorophenyl)-7-(prop-2-yn-1-yloxy)-1,2,3,4-tetrahydroisoquinoline 113 (33 mg, 0.117 mmol) in pyridine (0.25 mL) was added dropwise and stirring was continued for 72 hours. The reaction mixture was concentrated to dryness under reduced pressure. The residue was partitioned between chloroform (8 mL) and saturated aqueous K₂CO₃ (8 mL) and the layers were separated. The aqueous phase was extracted with chloroform (2×8 mL). The combined organic filtrates were passed through a phase separator and concentrated to dryness under reduced pressure. The residue was purified by flash column chromatography (silica, 0 to 10% (7M ammonia in methanol) in chloroform) to give (S)-1-(4-fluorophenyl)-7-(prop-2-yn-1-yloxy)-N—((S)-quinuclidin-3-yl)-3,4-dihydroisoquinoline-2(1H)-carboxamide compound 2115 after lyophilization from a mixture of acetonitrile and water (1:1, 4 mL).

Compound 2115 LCMS: 98%, RT=2.618 min., (M+H)⁺=434 (MC Method C).

¹H NMR (400 MHz, Chloroform-d) δ 7.26-7.19 (m, 2H), 7.12 (d, J=8.3 Hz, 1H), 7.01-6.93 (m, 2H), 6.87 (dd, J=8.3, 2.7 Hz, 1H), 6.76 (d, J=2.6 Hz, 1H), 6.29 (s, 1H), 4.64 (d, J=2.3 Hz, 2H), 4.54 (d, J=6.5 Hz, 1H), 3.92-3.83 (m, 1H), 3.65-3.52 (m, 2H), 3.36 (ddd, J=14.3, 9.5, 2.0 Hz, 1H), 2.92-2.70 (m, 6H), 2.48 (t, J=2.4 Hz, 1H), 2.44 (ddd, J=14.2, 5.0, 2.1 Hz, 1H), 1.89-1.82 (m, 1H), 1.70-1.58 (m, 2H), 1.50-1.32 (m, 2H).

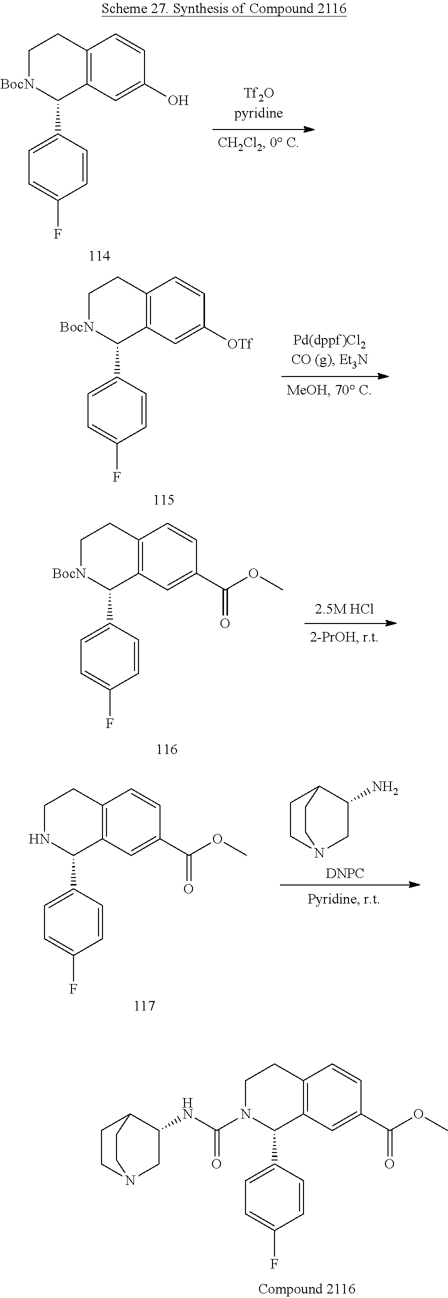

Step 1: At 0° C., pyridine (0.671 mL, 8.30 mmol) was added dropwise to a stirred solution of tert-butyl (S)-1-(4-fluorophenyl)-7-hydroxy-3,4-dihydroisoquinoline-2(1H)-carboxylate 114 (1.90 g, 5.53 mmol) in dichloromethane (40 mL) under nitrogen atmosphere. After 2 minutes, triflic anhydride (1.148 mL, 6.92 mmol) was added dropwise and stirring was continued for 1 hour. The reaction mixture was concentrated to dryness under reduced pressure and the residue was partitioned between ethyl acetate (25 mL) and water (25 mL). The aqueous was extracted with ethyl acetate (25 mL). The combined organics were washed with brine (25 mL), dried on Na₂SO₄, and evaporated under reduced pressure. The residue was purified by flash column chromatography (silica, 0 to 40% ethyl acetate in heptane) to give tert-butyl (S)-1-(4-fluorophenyl)-7-(((trifluoromethyl)sulfonyl)oxy)-3,4-dihydroisoquinoline-2(1H)-carboxylate 115 (2.145 g) as a colorless oil.

LCMS: 100%, RT=2.379 min., (M−tBu+2H)⁺=420 (MC Method A). ¹H NMR (400 MHz, Chloroform-d) δ 7.29 (d, J=8.5 Hz, 1H), 7.19-7.11 (m, 3H), 7.03-6.95 (m, 2H), 6.93 (s, 1H), 6.58-6.08 (m, 1H), 4.32-3.85 (m, 1H), 3.11 (ddd, J=13.1, 11.1, 4.0 Hz, 1H), 3.04-2.90 (m, 1H), 2.84-2.72 (m, 1H), 1.51 (s, 9H).

Step 2: Tert-butyl (S)-1-(4-fluorophenyl)-7-(((trifluoromethyl)sulfonyl)oxy)-3,4-dihydroisoquinoline-2(1H)-carboxylate 115 (1.98 g, 4.16 mmol), triethylamine (3.47 mL, 24.99 mmol) and 1,1′-bis(diphenylphosphino)ferrocenepalladium(II) dichloride (0.305 g, 0.416 mmol) were dissolved in methanol (anhydrous, 30 mL). The reaction mixture was stirred in an autoclave charged with 8 bars of carbon monoxide at 70° C. for 16 hours. The reaction mixture was concentrated to dryness under reduced pressure. The residue was purified by flash column chromatography (silica 0 to 35% ethyl acetate in heptane) to give 2-(tert-butyl) 7-methyl (S)-1-(4-fluorophenyl)-3,4-dihydroisoquinoline-2,7(1H)-dicarboxylate 116 (795 mg) as a colorless oil.

LCMS: 98%, RT=2.255 min., (M−tBu+2H)⁺=330 (MC Method A). ¹H NMR (400 MHz, Chloroform-d) δ 7.89 (dd, J=8.0, 1.8 Hz, 1H), 7.71 (s, 1H), 7.28 (d, J=8.0 Hz, 1H), 7.20-7.10 (m, 2H), 7.03-6.91 (m, 2H), 6.60-6.10 (m, 1H), 4.28-3.82 (m, 1H), 3.87 (s, 3H), 3.15 (ddd, J=13.1, 10.9, 4.1 Hz, 1H), 3.09-2.93 (m, 1H), 2.88-2.73 (m, 1H), 1.50 (s, 9H).

Step 3: A solution of hydrogen chloride in 2-propanol (5 M, 1.0 mL, 5.0 mmol) was added to a solution of 2-(tert-butyl) 7-methyl (S)-1-(4-fluorophenyl)-3,4-dihydroisoquinoline-2,7(1H)-dicarboxylate 116 (100 mg, 0.259 mmol) in 2-propanol (anhydrous, 1.0 mL) and stirred for 16 hours. The reaction mixture was concentrated to dryness under reduced pressure. The residue was partitioned between dichloromethane (6 mL) and saturated aqueous NaHCO₃ (4 mL) and the layers were separated. The aqueous phase was extracted with dichloromethane (2×6 mL). The combined organic filtrates were passed through a phase separator and concentrated to dryness under reduced pressure to give methyl (S)-1-(4-fluorophenyl)-1,2,3,4-tetrahydroisoquinoline-7-carboxylate 117 (107 mg) as a colorless oil.

LCMS: 97%, RT=1.453 min., (M+H)⁺=286 (MC Method A). ¹H NMR (400 MHz, Chloroform-d) δ 7.81 (dd, J=8.0, 1.8 Hz, 1H), 7.43 (s, 1H), 7.25-7.17 (m, 3H), 7.05-6.97 (m, 2H), 5.11 (s, 1H), 3.82 (s, 3H), 3.31-3.20 (m, 1H), 3.15-3.01 (m, 2H), 2.94-2.83 (m, 1H), 1.81 (br s, 1H).

Step 4: A solution of (S)-quinuclidin-3-amine (22.12 mg, 0.175 mmol) in pyridine (0.25 mL) was added dropwise to a stirred solution of bis(p-nitrophenyl) carbonate (53.3 mg, 0.175 mmol) in pyridine (0.50 mL) under nitrogen gas atmosphere. The reaction mixture was stirred for 2.5 hours, after which a solution of methyl (S)-1-(4-fluorophenyl)-1,2,3,4-tetrahydroisoquinoline-7-carboxylate 117 (50 mg, 0.175 mmol) in pyridine (0.50 mL) was added dropwise and stirring was continued for 16 hours. The reaction mixture was concentrated to dryness under reduced pressure. The residue was partitioned between chloroform (8 mL) and saturated aqueous NaHCO₃ (8 mL) and the layers were separated. The aqueous phase was extracted with chloroform (2×6 mL). The combined organic filtrates were passed through a phase separator and concentrated to dryness under reduced pressure. The residue was purified by flash column chromatography (silica, 0 to 10% (7 M ammonia in methanol) in chloroform) and basic preparative MPLC (Linear Gradient: t=0 min 5% B, t=1 min 5% B, t=2 min 10% B; t=17 min 50% B; t=18 min 100% B; t=23 min 100% B; detection: 220 nm). The product containing fractions were combined and lyophilised. The residue, containing residual dimethylsulfoxide from sample preparation, was dissolved in methanol (0.25 mL) and brought onto an SCX-2 column (1 g) and eluted with methanol (20 mL). Next, the column was eluted with ammonia in methanol (2 M). The basic fraction was concentrated to dryness under reduced pressure. The residue was lyophilised from a mixture of acetonitrile and water (1:1, 5 mL) to give methyl (S)-1-(4-fluorophenyl)-2-(((S)-quinuclidin-3-yl)carbamoyl)-1,2,3,4-tetrahydroisoquinoline-7-carboxylate compound 2116.

Compound 2116: LCMS: 98%, RT=2.594 min., (M+H)⁺=438 (MC Method C).

¹H NMR (400 MHz, Chloroform-d) δ 7.90 (dd, J=7.9, 1.8 Hz, 1H), 7.83 (d, J=1.7 Hz, 1H), 7.29-7.26 (m, 1H), 7.25-7.17 (m, 2H), 7.03-6.94 (m, 2H), 6.36 (s, 1H), 4.56 (d, J=6.4 Hz, 1H), 3.92-3.82 (m, 1H), 3.89 (s, 3H), 3.71-3.56 (m, 2H), 3.37 (ddd, J=14.2, 9.3, 2.0 Hz, 1H), 3.04-2.94 (m, 1H), 2.94-2.70 (m, 5H), 2.45 (ddd, J=14.0, 5.0, 2.1 Hz, 1H), 1.89-1.83 (m, 1H), 1.67-1.59 (m, 2H), 1.51-1.33 (m, 2H).

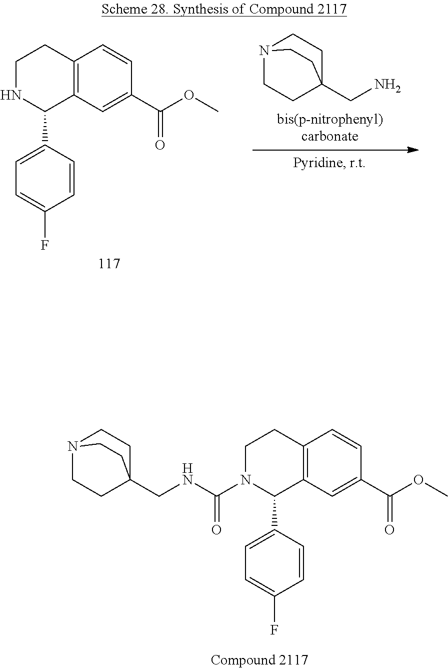 [SMILES: COC(=O)c1ccc2c(c1)[C@H](c1ccc(F)cc1)NCC2 ; NCC12CCN(CC1)CC2 ; COC(=O)c1ccc2c(c1)[C@H](c1ccc(F)cc1)N(C(=O)NCC13CCN(CC1)CC3)CC2]

A solution of quinuclidin-4-yl methanamine (24.57 mg, 0.175 mmol) in pyridine (0.25 mL) was added dropwise to a stirred solution of bis(p-nitrophenyl) carbonate (53.3 mg, 0.175 mmol) in pyridine (0.50 mL) under nitrogen atmosphere. The reaction mixture was stirred for 2.5 hours, after which a solution of methyl (S)-1-(4-fluorophenyl)-1,2,3,4-tetra-hydroisoquinoline-7-carboxylate 117 (50 mg, 0.175 mmol) in pyridine (0.50 mL) was added dropwise and stirring was continued for 16 hours. The reaction mixture was concentrated to dryness under reduced pressure. The residue was partitioned between chloroform (8 mL) and saturated aqueous NaHCO₃ (8 mL) and the layers were separated. The aqueous phase was extracted with chloroform (2×6 mL). The combined organic filtrates were passed through a phase separator and concentrated to dryness under reduced pressure. The residue was purified by flash column chromatography (silica, 0 to 10% (7 M ammonia in methanol) in chloroform) to give methyl (S)-1-(4-fluorophenyl)-2-((quinuclidin-4-ylmethyl)carbamoyl)-1,2,3,4-tetrahydroisoquinoline-7-carboxylate, compound 2117 after lyophilization from a mixture of acetonitrile and water (1:1, 5 mL).

Compound 2117: LCMS: 100%, RT=2.564 min., (M+H)⁺=452 (MC Method C).

¹H NMR (400 MHz, Chloroform-d) δ 7.91 (dd, J=8.0, 1.7 Hz, 1H), 7.84 (d, J=2.0 Hz, 1H), 7.29-7.25 (m, 1H), 7.24-7.16 (m, 2H), 7.03-6.94 (m, 2H), 6.38 (s, 1H), 4.48 (t, J=6.0 Hz, 1H), 3.89 (s, 3H), 3.61 (t, J=6.2 Hz, 2H), 3.12 (dd, J=13.6, 6.3 Hz, 1H), 3.08-2.93 (m, 2H), 2.93-2.79 (m, 7H), 1.37-1.22 (m, 6H).

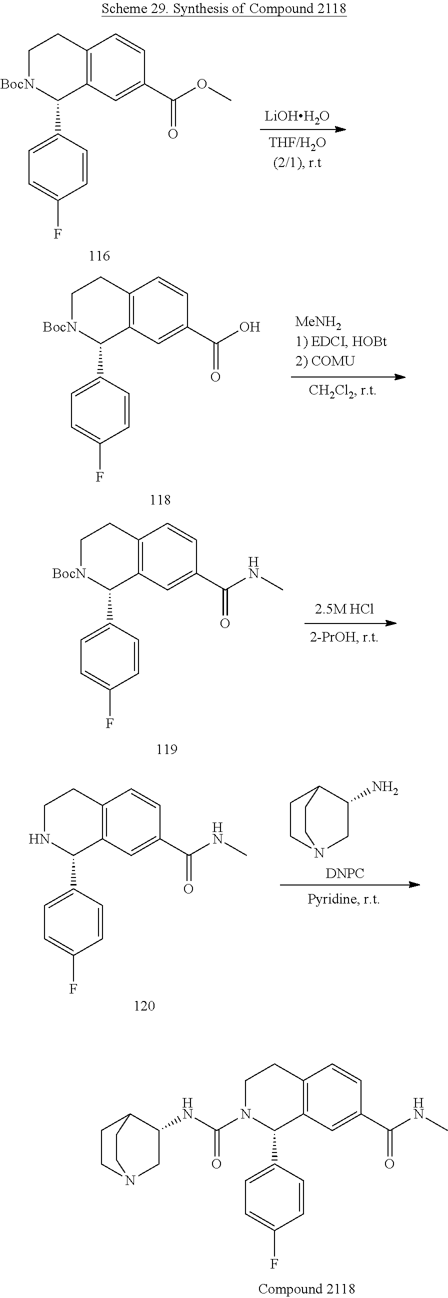

Step 1: A solution of lithium hydroxide monohydrate (45.3 mg, 1.079 mmol) in water (1.5 mL) was added to a solution of 2-(tert-butyl) 7-methyl (S)-1-(4-fluorophenyl)-3,4-dihydroisoquinoline-2,7(1H)-dicarboxylate 116 (208 mg, 0.540 mmol) in tetrahydrofuran (3.0 mL) and stirred for 40 hours. The reaction mixture was partitioned between dichloromethane (20 mL) and saturated aqueous NH₄Cl (15 mL). The layers were separated and the aqueous phase was extracted with dichloromethane (10 mL). The combined organic filtrates were passed through a phase separator and concentrated to dryness under reduced pressure to give (S)-2-(tert-butoxycarbonyl)-1-(4-fluorophenyl)-1,2,3,4-tetrahydroisoquinoline-7-carboxylic acid 118 (172.7 mg) as a colorless foam. LCMS: 100%, RT=2.281 min., (M−H)⁻=370 (MC Method A). ¹H NMR (400 MHz, Chloroform-d) δ 7.93 (dd, J=7.9, 1.8 Hz, 1H), 7.77 (s, 1H), 7.30 (d, J=8.1 Hz, 1H), 7.20-7.11 (m, 2H), 7.02-6.91 (m, 2H), 6.58-6.15 (m, 1H), 4.31-3.83 (m, 1H), 3.16 (ddd, J=13.2, 10.9, 4.2 Hz, 1H), 3.10-2.94 (m, 1H), 2.93-2.74 (m, 1H), 1.50 (s, 9H).

Step 2: N-(3-dimethylaminopropyl)-N′-ethylcarbodiimide hydrochloride (80 mg, 0.418 mmol) and 1-hydroxybenzotriazole hydrate (5.82 mg, 0.038 mmol) were added simultaneously to a stirred solution of (S)-2-(tert-butoxycarbonyl)-1-(4-fluorophenyl)-1,2,3,4-tetrahydroisoquinoline-7-carboxylic acid 118 (214 mg, 0.380 mmol) and methylamine (2 M in tetrahydrofuran, 0.190 mL, 0.380 mmol) in dichloromethane (2.0 mL). The reaction mixture was stirred for 16 hours, after which (1-cyano-2-ethoxy-2-oxoethyliden-aminooxy)dimethylamino-morpholino-carbenium hexafluorophosphate. (COMU, 326 mg, 0.761 mmol) was added and stirring was continued for an additional 1 hour. The reaction mixture was partitioned between dichloromethane (8 mL) and saturated aqueous NaHCO₃ (8 mL). The layers were separated and the aqueous phase was extracted with dichloromethane (8 mL). The combined organics were washed with brine (8 mL), dried on Na₂SO₄, and evaporated under reduced pressure. The residue was purified by flash column chromatography (silica, 0 to 60% ethyl acetate in heptane) to give tert-butyl (S)-1-(4-fluorophenyl)-7-(methylcarbamoyl)-3,4-dihydroisoquinoline-2(1H)-carboxylate 119 (181 mg) as a white solid which was used as such. LCMS: 87%, RT=2.110 min., (M+Na)⁺=407 (MC Method A). ¹H NMR (400 MHz, Chloroform-d) δ 7.71-7.53 (m, 1H), 7.42 (s, 1H), 7.26 (d, J=8.1 Hz, 1H), 7.21-7.08 (m, 2H), 7.01-6.90 (m, 2H), 6.57-6.11 (m, 1H), 6.09-6.00 (m, 1H), 4.29-3.84 (m, 1H), 3.13 (ddd, J=13.2, 10.9, 4.3 Hz, 1H), 3.08-2.90 (m, 4H), 2.84-2.73 (m, 1H), 1.50 (s, 9H).

Step 3: A solution of hydrogen chloride in 2-propanol (5 M, 1.0 mL, 5.0 mmol) was added to a solution of tert-butyl (S)-1-(4-fluorophenyl)-7-(methylcarbamoyl)-3,4-dihydroisoquinoline-2(1H)-carboxylate 119 (170 mg, 0.442 mmol) in 2-propanol (anhydrous, 1.0 mL) and stirred for 16 hours. The reaction mixture was concentrated to dryness under reduced pressure. The residue was partitioned between dichloromethane (8 mL) and saturated aqueous NaHCO₃ (8 mL) and the layers were separated. The aqueous phase was extracted with dichloromethane (2×6 mL). The combined organic filtrates were passed through a phase separator and concentrated to dryness under reduced pressure to give (S)-1-(4-fluorophenyl)-N-methyl-1,2,3,4-tetrahydroisoquinoline-7-carboxamide 120 (124 mg) as a colorless solid. LCMS: 97%, RT˜0.1-0.45 min. (broad signal), (M+H)⁺=285 (MC Method A). ¹H NMR (400 MHz, Chloroform-d) δ 7.54 (dd, J=8.0, 1.9 Hz, 1H), 7.25-7.17 (m, 3H), 7.13 (d, J=1.9 Hz, 1H), 7.05-6.97 (m, 2H), 5.91 (br s, 1H), 5.10 (s, 1H), 3.32-3.21 (m, 1H), 3.14-2.98 (m, 3H), 2.93 (d, J=4.9 Hz, 3H), 2.89-2.81 (m, 1H).

Step 4: A solution of (S)-quinuclidin-3-amine (24.37 mg, 0.193 mmol) in pyridine (0.25 mL) was added dropwise to a stirred solution of bis(p-nitrophenyl) carbonate (58.7 mg, 0.193 mmol) in pyridine (0.50 mL) under nitrogen gas atmosphere. The reaction mixture was stirred for 2.5 hours, after which a solution of (S)-1-(4-fluorophenyl)-N-methyl-1,2,3,4-tetrahydroisoquinoline-7-carboxamide 120 (61 mg, 0.193 mmol) in pyridine (0.50 mL) was added dropwise and stirring was continued for 72 hours. The reaction mixture was concentrated to dryness under reduced pressure. The residue was partitioned between chloroform (10 mL) and aqueous NaOH (0.5 M, 6.5 mL) and the layers separated. The organic layer was washed with aqueous NaOH (0.5 M, 6.5 mL), passed through a phase separator, and concentrated to dryness under reduced pressure. The residue was purified by flash column chromatography (silica, 0 to 10% (7M ammonia in methanol) in chloroform) to give (S)-1-(4-fluorophenyl)-N7-methyl-N2-((S)-quinuclidin-3-yl)-3,4-dihydroisoquinoline-2,7(1H)-dicarboxamide Compound 2118 after lyophilization from a mixture of acetonitrile and water (1:1, 4 mL).

Compound 2118: LCMS: 100%, RT=2.281 min., (M+H)⁺=437 (MC Method C).

¹H NMR (400 MHz, Chloroform-d) δ 7.64 (dd, J=7.9, 1.9 Hz, 1H), 7.53 (d, J=1.9 Hz, 1H), 7.28-7.24 (m, 1H), 7.23-7.15 (m, 2H), 7.02-6.93 (m, 2H), 6.38 (s, 1H), 6.16-6.08 (m, 1H), 4.59 (d, J=6.5 Hz, 1H), 3.90-3.82 (m, 1H), 3.68-3.53 (m, 2H), 3.36 (ddd, J=14.2, 9.6, 2.0 Hz, 1H), 2.98 (d, J=4.8 Hz, 3H), 2.94 (dd, J=7.8, 5.5 Hz, 1H), 2.89 (t, J=5.6 Hz, 1H), 2.86-2.70 (m, 4H), 2.45 (ddd, J=14.1, 5.0, 2.1 Hz, 1H), 1.89-1.82 (m, 1H), 1.67-1.59 (m, 2H), 1.52-1.32 (m, 2H).

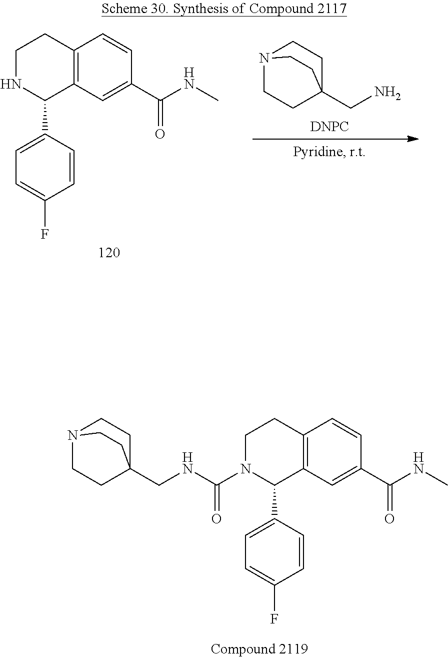

A solution of quinuclidin-4-ylmethanamine (27.1 mg, 0.193 mmol) in pyridine (0.25 mL) was added dropwise to a solution of bis(p-nitrophenyl) carbonate (58.7 mg, 0.193 mmol) in pyridine (0.50 mL) under nitrogen atmosphere. The reaction mixture was stirred for 2.5 hours, after which a solution of (S)-1-(4-fluorophenyl)-N-methyl-1,2,3,4-tetrahydroisoquinoline-7-carboxamide 120 (61 mg, 0.193 mmol) in pyridine (0.50 mL) was added dropwise and stirring was continued for 72 hours. The reaction mixture was concentrated to dryness under reduced pressure. The residue was partitioned between chloroform (10 mL) and aqueous NaOH (0.5 M, 6.5 mL) and the layers separated. The organic layer was washed with aqueous NaOH (0.5 M, 6.5 mL), passed through a phase separator, and concentrated to dryness under reduced pressure. The residue was purified by flash column chromatography (silica, 0 to 20% (7M ammonia in methanol) in chloroform) to give (S)-1-(4-fluorophenyl)-N7-methyl-N2-(quinuclidin-4-ylmethyl)-3,4-dihydroisoquinoline-2,7(1H)-dicarboxamide Compound 2119 after lyophilization from a mixture of acetonitrile and water (1:1, 4 mL).

Compound 2119: LCMS: 97%, RT=2.264 min., (M+H)⁺=451 (MC Method C).

¹H NMR (400 MHz, Chloroform-d) δ 7.64 (dd, J=7.9, 1.9 Hz, 1H), 7.54 (d, J=1.8 Hz, 1H), 7.27-7.24 (m, 1H), 7.23-7.14 (m, 2H), 7.01-6.92 (m, 2H), 6.39 (s, 1H), 6.18-6.06 (m, 1H), 4.50 (t, J=6.1 Hz, 1H), 3.65-3.53 (m, 2H), 3.11 (dd, J=13.6, 6.2 Hz, 1H), 3.03 (dd, J=13.6, 5.8 Hz, 1H), 2.99 (d, J=4.9 Hz, 3H), 2.94 (t, J=6.7 Hz, 1H), 2.91-2.78 (m, 7H), 1.36-1.22 (m, 6H).

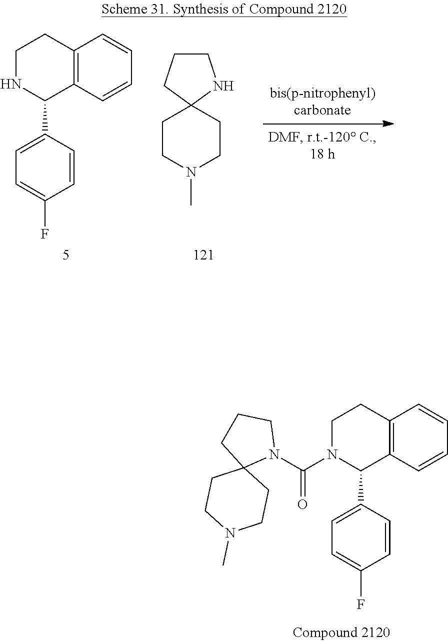

(S)-1-(4-Fluorophenyl)-1,2,3,4-tetrahydroisoquinoline 5 (50 mg, 0.220 mmol) was dissolved in N,N-dimethylformamide (2 mL). Bis(p-nitrophenyl) carbonate (67 mg, 0.220 mmol) was added and the mixture was stirred overnight. 8-Methyl-1,8-diazaspiro[4.5]decane 121 (33.9 mg, 0.220 mmol) was added and the mixture was stirred at room temperature overnight. Another portion of 8-Methyl-1,8-diazaspiro[4.5]decane 121 (33.9 mg, 0.220 mmol) was added and the mixture was stirred at 60° C. for 2 hours and at 120° C. overnight. The mixture was cooled to room temperature and concentrated under reduced pressure. The residue was purified by flash column chromatography (silica, 0 to 5% methanol in dichloronethane) to obtain 40 mg of a yellow solid. The product was purified by a SCX-2 column (1 g) to obtain (S)-(1-(4-fluorophenyl)-3,4-dihydroisoquinolin-2(1H)-yl)(8-methyl-1,8-diazaspiro[4.5]decan-1-yl)methanone compound 2120: (30 mg) as a yellow solid.

Compound 2120: LCMS: 98%, RT=1.348 min., (M+H)⁺=408 (MC Method E).

¹H NMR (300 MHz, Chloroform-d) δ 7.25-7.12 (m, 6H), 7.03-6.88 (m, 3H), 6.19 (s, 1H), 3.66 (d, J=13.2 Hz, 1H), 3.61-3.47 (m, 2H), 3.46-3.24 (m, 3H), 3.22 (s, 2H), 3.01 (ddd, J=16.3, 10.5, 6.2 Hz, 1H), 2.81 (d, J=16.1 Hz, 1H), 2.42 (s, 1H), 2.26 (s, 4H), 1.69 (q, J=7.4, 6.8 Hz, 2H), 1.25 (s, 1H).

The following compound was prepared analogously using 8-methyl-2,8-diazaspiro[4.5]decane:

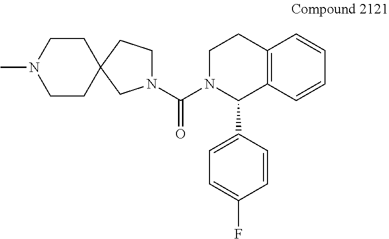 [SMILES: CN1CCC2(CC1)CCN(C(=O)N1CCc3ccccc3[C@@H]1c1ccc(F)cc1)C2]

Compound 2121: LCMS: 97%, RT=1.347 min., (M+H)⁺=408 (MC Method E).

¹H NMR (300 MHz, Chloroform-d) δ 7.25-7.09 (m, 5H), 7.03-6.90 (m, 3H), 6.19 (s, 1H), 3.72-3.62 (m, 1H), 3.61-3.25 (m, 6H), 3.22 (s, 2H), 3.01 (ddd, J=16.5, 10.3, 5.9 Hz, 2H), 2.88-2.75 (m, 1H), 2.27 (s, 4H), 1.76-1.56 (m, 5H), 1.25 (s, 2H).

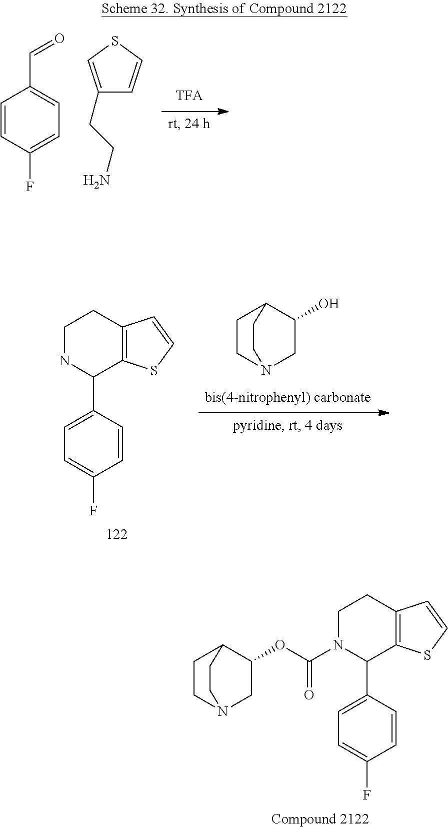

Step 1: 4-Fluorobenzaldehyde (0.140 mL, 1.30 mmol) and 2-thiophen-3-ylethylamine (165 mg, 1.30 mmol) were combined and stirred at room temperature for 4 hours. Trifluoroacetic acid (0.500 mL 6.49 mmol) was added and the resulting solution was stirred at room temperature for 20 hours. The mixture was concentrated under reduced pressure. The residue was purified by flash column chromatography (silica, 10 to 100% ethyl acetate in heptane) to obtain 7-(4-fluorophenyl)-4,5,6,7-tetrahydrothieno[2,3-c]pyridine 122 (140 mg) as a white solid.

LCMS: 97%, RT=1.32 min., (M+H)⁺=234 (MC Method A). ¹H NMR (400 MHz, chloroform-d) δ 7.37-7.29 (m, 2H), 7.14 (d, J=5.1 Hz, 1H), 7.07-6.97 (m, 2H), 6.83 (d, J=5.1 Hz, 1H), 5.17-5.11 (m, 1H), 3.33-3.22 (m, 1H), 3.13-3.01 (m, 1H), 2.89-2.77 (m, 1H), 2.75-2.64 (m, 1H).

Step 2: Under an argon atmosphere, a solution of (S)-(+)-3-quinuclidinol (54.5 mg, 0.429 mmol) and bis(p-nitrophenyl) carbonate (130 mg, 0.429 mmol) in pyridine (2 mL) was stirred at room temperature for 4 hours. 7-(4-fluorophenyl)-4,5,6,7-tetrahydrothieno[2,3-c]pyridine 122 (100 mg, 0.429 mmol) in pyridine (1 mL) was added and stirring was continued for 4 days. The mixture was poured into a mixture of ice and saturated aqueous K₂CO₃ (1:1, 25 mL) and extracted with ethyl acetate (2×20 mL). The combined organic layer was dried over Na₂SO₄ and concentrated under reduced pressure. Purification by flash column chromatography (silica, 0 to 10% (7 M ammonia in methanol) in chloroform) afforded (S)-quinuclidin-3-yl 7-(4-fluorophenyl)-4,7-dihydrothieno[2,3-c]pyridine-6(5H)-carboxylate compound 2122 after lyophilisation, as a mixture of diastereoisomers.

Compound 2122: LCMS: 100%, RT=2.66 min., (M+H)⁺=387 (MC Method C).

¹H NMR (400 MHz, chloroform-d) δ 7.37-7.28 (m, 2H), 7.24 (d, J=5.1 Hz, 1H), 7.04-6.95 (m, 2H), 6.85 (d, J=5.1 Hz, 1H), 6.66-6.14 (m, 1H), 4.87-4.71 (m, 1H), 4.53-4.01 (m, 1H), 3.34-3.19 (m, 1H), 3.18-3.06 (m, 1H), 2.96-2.61 (m, 7H), 2.10-2.02 (m, 1H), 1.90-1.75 (m, 1H), 1.75-1.64 (m, 1H), 1.63-1.50 (m, 1H), 1.49-1.36 (m, 1H).

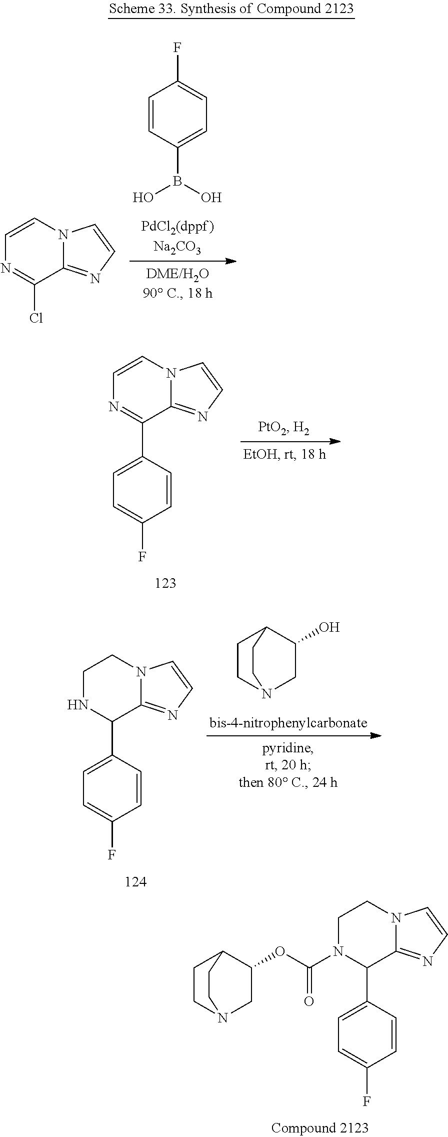 [SMILES: OB(O)c1ccc(F)cc1 ; Clc1nccn2ccnc12 ; Fc1ccc(-c2nccn3ccnc23)cc1 ; Fc1ccc(C2NCCn3ccnc32)cc1 ; O[C@@H]1CN2CCC1CC2 ; O=C(O[C@@H]1CN2CCC1CC2)N1CCn2ccnc2C1c1ccc(F)cc1]

Step 1: A suspension of 4-fluorophenylboronic acid (501 mg, 3.58 mmol), 8-chloroimidazo[1,2-a]pyrazine (500 mg, 3.26 mmol), and sodium carbonate (1.04 g, 9.77 mmol) in 1,2-dimethoxyethane (10 mL) and water (2 mL) was flushed with argon. [1,1′-Bis(diphenylphosphino)ferrocene]palladium(II) dichloride (119 mg, 0.163 mmol) was added and the mixture was heated at 90° C. for 18 hours. The mixture was diluted with water (50 mL) and extracted with ethyl acetate (2×30 mL). The combined organic layer was washed with brine (25 mL), dried over Na₂SO₄, and concentrated under reduced pressure. The residue was purified by flash column chromatography (silica, 0 to 50% ethyl acetate in heptane) to obtain 8-(4-fluorophenyl)imidazo[1,2-a]pyrazine 123 (503 mg) as a white solid.

LCMS: 100%, RT=1.82 min., (M+H)⁺=214 (MC Method B). ¹H NMR (400 MHz, chloroform-d) δ 8.80-8.71 (m, 2H), 8.06 (d, J=4.5 Hz, 1H), 7.97 (d, J=4.4 Hz, 1H), 7.86 (d, J=1.2 Hz, 1H), 7.74 (d, J=1.2 Hz, 1H), 7.26-7.18 (m, 2H).

Step 2: Platinum (IV) oxide (26.6 mg, 0.117 mmol) was added to a solution of 8-(4-fluorophenyl)imidazo[1,2-a]pyrazine 123 (250 mg, 1.173 mmol) in ethanol. The mixture was stirred under a hydrogen atmosphere for 18 hours. The mixture was filtered over Celite and the filtrate was concentrated under reduced pressure to obtain 8-(4-fluorophenyl)-5,6,7,8-tetrahydroimidazo[1,2-a]pyrazine 124 (264 mg, 1.173 mmol) as a syrup. ¹H NMR (400 MHz, chloroform-d) δ 7.43-7.31 (m, 2H), 7.09-6.94 (m, 3H), 6.90-6.79 (m, 1H), 5.18 (s, 1H), 4.13 (ddd, J=11.9, 8.7, 4.7 Hz, 1H), 4.02 (dt, J=11.9, 4.3 Hz, 1H), 3.37 (dt, J=12.9, 4.5 Hz, 1H), 3.27 (ddd, J=12.9, 8.7, 4.4 Hz, 1H).

Step 3: Under an argon atmosphere, a solution of (S)-(+)-3-quinuclidinol (54.5 mg, 0.429 mmol) and bis(p-nitrophenyl) carbonate (130 mg, 0.429 mmol) in pyridine (2 mL) was stirred at room temperature for 4 hours. 8-(4-Fluorophenyl)-5,6,7,8-tetrahydroimidazo[1,2-a]pyrazine 124 (100 mg, 0.428 mmol) in pyridine (1 mL) was added and stirring was continued for 4 days. The mixture was heated at 80° C. for 20 hours. The mixture was poured into a mixture of ice and saturated aqueous K₂CO₃ (1:1, 25 mL) and extracted with ethyl acetate (2×20 mL). The combined organic layer was dried over Na₂SO₄ and concentrated under reduced pressure. Purification by flash column chromatography (silica, 0 to 10% (7M ammonia in methanol) in chloroform) afforded (S)-quinuclidin-3-yl 8-(4-fluorophenyl)-5,6-dihydroimidazo[1,2-a]pyrazine-7(8H)-carboxylate compound 2123 after lyophilisation, mixture of diastereoisomers.

Compound 2123: LCMS: 99%, RT=2.85 min., (M+H)⁺=371 (MC Method D).

¹H NMR (400 MHz, chloroform-d) δ 7.35-7.27 (m, 2H), 7.14-7.11 (m, 1H), 7.08-6.97 (m, 2H), 6.95-6.88 (m, 1H), 6.48 (s, 1H), 4.88-4.79 (m, 1H), 4.59-4.26 (m, 1H), 4.16-4.07 (m, 1H), 4.07-3.97 (m, 1H), 3.44-3.20 (m, 2H), 2.96-2.67 (m, 5H), 2.22-1.97 (m, 1H), 1.83-1.66 (m, 2H), 1.65-1.50 (m, 1H), 1.49-1.36 (m, 1H).

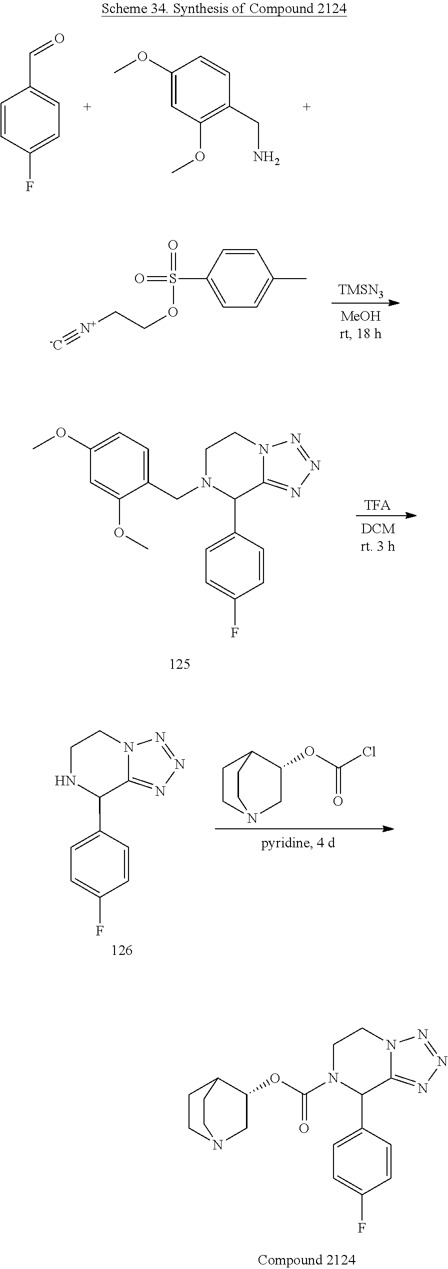

Step 1: A solution of 2,4-dimethoxybenzylamine (1.00 mL, 6.66 mmol) and 4-fluorobenzaldehyde (0.719 mL, 6.66 mmol) in methanol (20 mL) was stirred at room temperature for 3 hours. 2-Isocyanoethyl 4-methylbenzenesulfonate (1.00 g, 4.44 mmol) and azidotrimethylsilane (0.584 mL, 4.44 mmol) were added and stirring was continued at room temperature for 18 hours. The mixture was concentrated under reduced pressure. The residue was dissolved in dichloromethane and washed with saturated aqueous NaHCO₃. The organic layer was dried over Na₂SO₄ and concentrated under reduced pressure. The residue was purified by flash column chromatography (silica, 5 to 60% ethyl acetate in heptane) to obtain 7-(2,4-dimethoxybenzyl)-8-(4-fluorophenyl)-5,6,7,8-tetrahydrotetrazolo[1,5-a]pyrazine 125 (1.01 g) as a white solid.

LCMS: 97%, RT=2.05 min. (M+H)⁺=370 (MC Method A). ¹H NMR (400 MHz, Chloroform-dl) δ 7.48-7.39 (m, 2H), 7.15 (d, J=8.2 Hz, 1H), 7.12-7.04 (m, 2H), 6.51-6.41 (m, 2H), 4.95 (s, 1H), 4.53 (dt, J=12.7, 4.0 Hz, 1H), 4.38 (ddd, J=13.0, 8.9, 4.6 Hz, 1H), 3.81 (s, 3H), 3.77-3.69 (m, 4H), 3.55 (d, J=13.4 Hz, 1H), 3.40 (dt, J=13.3, 4.4 Hz, 1H), 2.88 (ddd, J=13.3, 9.1, 4.1 Hz, 1H).

Step 2: Trifluoroacetic acid (1.00 ml, 13.00 mmol) was added to a solution of 7-(2,4-dimethoxybenzyl)-8-(4-fluorophenyl)-5,6,7,8-tetrahydrotetrazolo[1,5-a]pyrazine 125 (500 mg, 1.35 mmol) in dichloromethane (15 mL). The mixture was stirred at room temperature for 3 hours. The mixture was poured into water, carefully saturated aqueous NaHCO₃ was added (20 mL). Dichloromethane (30 mL) was added, the layers were separated, the organic layer was washed with water and brine, dried over Na₂SO₄ and concentrated under reduced pressure to obtain 8-(4-fluorophenyl)-5,6,7,8-tetrahydrotetrazolo[1,5-a]pyrazine 126 (320 mg, 1.35 mmol) as a waxy white solid. ¹H NMR (400 MHz, DMSO-d₆) δ 7.48-7.38 (m, 2H), 7.26-7.15 (m, 2H), 5.40 (d, J=5.1 Hz, 1H), 4.50-4.35 (m, 2H), 3.52-3.39 (m, 1H), 3.31-3.13 (m, 2H).

Step 3: (S)-quinuclidin-3-yl carbonochloridate hydrochloride (45 mg, 0.199 mmol) was added to a suspension of 8-(4-fluorophenyl)-5,6,7,8-tetrahydrotetrazolo[1,5-a]pyrazine 126 (30 mg, 0.137 mmol) in pyridine (1 mL). The mixture was stirred at room temperature for 4 days. Additional (S)-quinuclidin-3-yl carbonochloridate hydrochloride (30.9 mg, 0.137 mmol) was added and stirring was continued for 1 hour. The mixture was concentrated under reduced pressure. Dimethylsulfoxide (2 mL) was added to the residue, the solids were filtered off and the filtrate was purified by basic preparative MPLC (Phenomenex Gemini C18, Linear Gradient: t=0 min 5% A, t=1 min 5% A, t=2 min 10% A; t=20 min 50% A; t=21 min 100%; t=24 min 100% A; detection: 220/254 nm) followed by lyophilization to obtain 12 mg of (S)-quinuclidin-3-yl 8-(4-fluorophenyl)-5,6-dihydrotetrazolo[1,5-a]pyrazine-7(8H-carboxylate Compound 2124 as a white amorphous solid.

Compound 2124 LCMS: 97%, RT=2.97 min., (M+H)⁺=373 (MC Method J).

¹H NMR (400 MHz, DMSO-d₆) δ 7.51-7.34 (m, 2H), 7.31-7.14 (m, 2H), 6.70-6.61 (m, 1H), 4.72-4.64 (m, 2H), 4.55-4.36 (m, 2H), 3.58-3.41 (m, 1H), 3.10-2.98 (m, 1H), 2.80-2.54 (m, 5H), 2.01-1.83 (m, 1H), 1.78-1.51 (m, 2H), 1.51-1.38 (m, 1H), 1.39-1.15 (m, 1H).

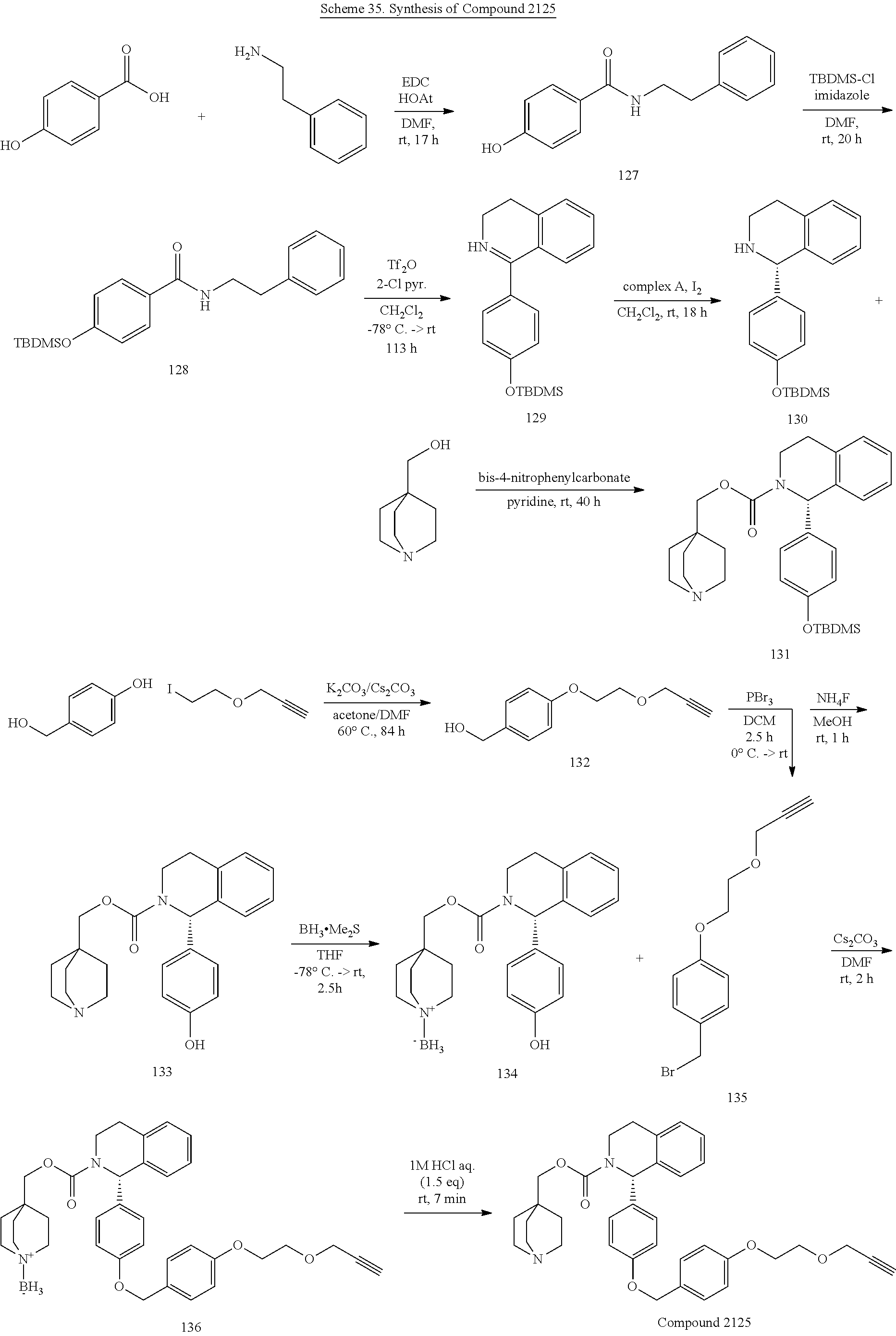

Step 1: Under nitrogen atmosphere, 4-hydroxybenzoic acid (9.03 g, 62.1 mmol) was dissolved in N,N-dimethylformamide (dry, 90 mL) with mild heating. Next, phenethylamine (7.90 mL, 62.1 mmol) was slowly added to the solution. During the addition, the reaction mixture was briefly cooled in an ice bath to maintain the temperature of the reaction mixture close to room temperature. Then, N-(3-dimethylaminopropyl)-N′-ethylcarbodiimide hydrochloride (11.9 g, 62.1 mmol) was added portion-wise followed by the addition of 1-hydroxy-7-azabenzotriazole (0.845 g, 6.21 mmol). The resulting yellow solution was stirred at room temperature for 16 hours and then slowly poured into ice-cold water (500 mL) while stirring. The resulting suspension was stirred for another 15 minutes. The formed solid was filtered off, washed with ice-cold water (3×70 mL), and dried under reduced pressure to give 4-hydroxy-N-phenethylbenzamide 127 (11.9 g) as a pale yellow powder.

LCMS: 97%, RT=1.78 min, (M+H)⁺=242 (MC Method A). ¹H NMR (400 MHz, DMSO-d₆) δ 9.94 (br s, 1H), 8.28 (t, J=5.6 Hz, 1H), 7.68 (d, J=8.6 Hz, 2H), 7.34-7.25 (m, 2H), 7.25-7.15 (m, 3H), 6.77 (d, J=8.6 Hz, 2H), 3.49-3.38 (m, 2H), 2.81 (t, J=7.5 Hz, 2H).

Step 2: Tert-butyldimethylsilyl chloride (9.62 g, 63.8 mmol) was added to a solution of 4-hydroxy-N-phenethylbenzamide 127 (11.85 g, 49.1 mmol) and imidazole (8.36 g, 123 mmol) in dichloromethane (100 mL) and N,N-dimethylformamide (20 mL) and the resulting suspension was stirred at room temperature for 20 hours under nitrogen. Over the course of reaction additional imidazole (4.68 g, 68.8 mmol) and tert-butyldimethylsilyl chloride (5.18 g, 34.4 mmol) were added to the reaction mixture. Next, dichloromethane was removed under reduced pressure and the remaining turbid solution was slowly poured into water (450 mL) while stirring. The formed suspension was stirred for 45 minutes. The formed solid was filtered off, washed with water (3×50 mL), and then dissolved in dichloromethane (100 mL) and dried over Na₂SO₄. The solids were filtered off and the filtrate was concentrated under reduced pressure. The residue was purified by crystallization from hot methanol (7.5 mL). The formed crystals were filtered off and washed with cold methanol (2×8 mL) to give 4-((tert-butyldimethylsilyl)oxy)-N-phenethylbenzamide 128 (12.25 g) as white crystals.

LCMS: 99%, RT=2.34 min, (M+H)⁺=356 (MC Method A). ¹H NMR. ¹H NMR (400 MHz, Chloroform-d) δ 7.61-7.55 (m, 2H), 7.36-7.29 (m, 2H), 7.28-7.20 (m, 3H), 6.86-6.79 (m, 2H), 6.03 (t, J=5.9 Hz, 1H), 3.70 (q, J=6.8 Hz, 2H), 2.92 (t, J=6.9 Hz, 2H), 0.98 (s, 9H), 0.20 (s, 6H).

Step 3: Under argon atmosphere at −78° C., triflic anhydride (6.95 mL, 41.3 mmol) was added dropwise to a solution of 2-chloropyridine (4.20 mL, 44.8 mmol) and 4-((tert-butyldimethylsilyl)oxy)-N-phenethylbenzamide 128 (12.24 g, 34.4 mmol) in dichloromethane (180 mL). Afterwards, the reaction mixture was allowed to warm to room temperature and was stirred for 113 h. Then, aqueous NaOH (2 M, 150 mL) was added and the reaction mixture was stirred vigorously for 10 min. The layers were separated and the organic layer was dried over Na₂SO₄, filtered, and concentrated under reduced pressure. The residue was purified by flash column chromatography (silica, 3 to 25% ethyl acetate in heptane) to give 1-(4-((tert-butyldimethylsilyl)oxy)phenyl)-3,4-dihydroisoquinoline 129 (9.42 g) as a thick yellow oil.

LCMS: 99%, RT=1.87 min, (M+H)⁺=338 (MC Method A). ¹H NMR (400 MHz, Chloroform-d) δ 7.53-7.46 (m, 2H), 7.41-7.33 (m, 1H), 7.33-7.21 (m, 3H), 6.89-6.85 (m, 2H), 3.84-3.76 (m, 2H), 2.82-2.74 (m, 2H), 1.00 (s, 9H), 0.22 (s, 6H).

Step 4: To a solution of 1-(4-((tert-butyldimethylsilyl)oxy)phenyl)-3,4-dihydroisoquinoline 129 (4.00 g, 11.26 mmol) in dichloromethane (60 mL), iodine (0.086 g, 0.338 mmol) and subsequently [{lr(H)[(S,S)-(f)-Binaphane]}₂(μ-I)₃]⁺I⁻ (Complex A, 0.028 g, 0.011 mmol) were added. The resulting suspension was stirred in an autoclave charged with 40 bars of hydrogen gas at room temperature for 18 hours. Next, the reaction mixture was filtered through a pad of Celite, washed with dichloromethane and the solvent was removed from the filtrate under reduced pressure. The crude product was purified by crystallization from a hot methanol/water mixture. The formed crystals were filtered off, washed with a mixture of cold water and methanol (9:1) and dried in vacuum to give (S)-1-(4-((tert-butyldimethylsilyl)oxy)phenyl)-1,2,3,4-tetrahydroisoquinoline 130 (1.38 g) as pale brown crystals.

LCMS: 100%, RT=1.84 min, (M+H)⁺=340 (MC Method A). Chiral LC: RT=4.99 min., 98% ee (MC Method H). ¹H NMR (400 MHz, Chloroform-d) δ 7.16-7.08 (m, 4H), 7.07-6.99 (m, 1H), 6.82-6.72 (m, 3H), 5.04 (s, 1H), 3.32-3.21 (m, 1H), 3.14-2.97 (m, 2H), 2.87-2.76 (m, 1H), 1.97-1.83 (br s, 1H), 0.98 (s, 9H), 0.19 (s, 6H).

Step 5: Under nitrogen atmosphere, bis(p-nitrophenyl) carbonate (1077 mg, 3.54 mmol) was added to a solution of quinuclidin-4-ylmethanol (500 mg, 3.54 mmol) in pyridine (35 mL) and the resulting yellow solution was stirred at room temperature for 16 hours. Then, (S)-1-(4-((tert-butyldimethylsilyl)oxy)phenyl)-1,2,3,4-tetrahydroisoquinoline 130 (1202 mg, 3.54 mmol) was added to the reaction mixture and stirring was continued for another 24 hours. Next, pyridine was removed under reduced pressure and the residue was purified by flash column chromatography (silica, 0 to 8% (7 M ammonia in methanol) in chloroform) to give quinuclidin-4-ylmethyl (S)-1-(4-((tert-butyldimethylsilyl)oxy)phenyl)-3,4-dihydroisoquinoline-2(1H)-carboxylate 131 (1.49 g) as a thick pale brown oil.

LCMS: 98%, RT=2.00 min, (M+H)⁺=507 (MC Method A). ¹H NMR (400 MHz, Chloroform-d) mixture of rotamers δ 7.25-7.14 (m, 3H), 7.09-7.00 (m, 3H), 6.76-6.69 (m, 2H), 6.47-6.08 (m, 1H), 4.18-3.91 (m, 1H), 3.91-3.76 (m, 2H), 3.30-3.19 (m, 1H), 3.04-2.84 (m, 7H), 2.77 (dt, J=16.2, 4.0 Hz, 1H), 1.48-1.37 (m, 6H), 0.96 (s, 9H), 0.17 (s, 6H).

Step 6: Ammonium fluoride (0.636 g, 17.17 mmol) was added to a solution of quinuclidin-4-ylmethyl (S)-1-(4-((tert-butyldimethylsilyl)oxy)phenyl)-3,4-dihydroisoquinoline-2(1H)-carboxylate 131 (1.45 g, 2.86 mmol) in methanol (15 mL) and the resulting suspension was stirred at room temperature for 1 hour. Then, methanol was removed under reduced pressure and the resulting solid was dissolved in chloroform (200 mL), washed with saturated aqueous NaHCO₃ (40 mL), and the aqueous layer was extracted with chloroform (3×80 mL). The combined organic layers were dried over Na₂SO₄, filtered, and concentrated under reduced pressure to give quinuclidin-4-ylmethyl (S)-1-(4-hydroxyphenyl)-3,4-dihydroisoquinoline-2(1H)-carboxylate 133 (1.12 g) as an of white foam. The crude product was used as such in the following step.

LCMS: 100%, RT=1.62 min, (M+H)⁺=393 (MC Method A). ¹H NMR (400 MHz, Chloroform-d) mixture of rotamers δ 7.23-7.11 (m, 3H), 7.11-7.03 (m, 3H), 6.68-6.61 (m, 2H), 6.50-5.95 (m, 1H), 4.30-3.50 (m, 3H), 3.50-3.20 (m, 1H), 3.03-2.75 (m, 8H), 1.58-1.33 (m, 6H).

Step 7: Under argon atmosphere, borane-dimethyl sulfide complex (2 M, 1.541 mL, 3.08 mmol) was added to a suspension of quinuclidin-4-ylmethyl (S)-1-(4-hydroxyphenyl)-3,4-dihydroisoquinoline-2(1H)-carboxylate 133 (1.10 g, 2.80 mmol) in dry tetrahydrofuran (25 mL) at −78° C. The reaction mixture was stirred for 1 hour after which additional borane-dimethyl sulfide complex (2 M, 0.210 mL, 0.420 mmol) was added. Then, the reaction mixture was allowed to warm to room temperature and stirring was continued for 1.5 hour. Next, methanol (0.7 mL) was added and after stirring briefly, the solvents were removed under reduced pressure. The residue was purified by flash column chromatography (silica, 2 to 50% ethyl acetate in heptane) to give (quinuclidin-4-ylmethyl (S)-1-(4-hydroxyphenyl)-3,4-dihydroisoquinoline-2(1H)-carboxylate)trihydroborate 134 (0.81 g) as a white foam.

LCMS: 100%, RT=2.08 min, (M+H)⁺=405 (MC Method A). ¹H NMR (400 MHz, Chloroform-d) mixture of rotamers δ 7.25-7.15 (m, 3H), 7.10-6.98 (m, 3H), 6.74 (d, J=8.2 Hz, 2H), 6.50-6.00 (br s, 1H), 5.07-4.90 (m, 1H), 4.07-3.87 (m, 3H), 3.36-3.24 (m, 1H), 3.05-2.91 (m, 7H), 2.80 (dt, J=16.3, 4.2 Hz, 1H), 1.68-1.56 (m, 6H), 1.89-1.17 (br s, 3H, BH₃).

Step 8: Potassium carbonate (1.123 g, 8.13 mmol) was added to a solution of 4-(hydroxymethyl)phenol (1.03 g, 8.05 mmol) in acetone (17 mL) and the resulting suspension was stirred for 5 minutes. A solution of 3-(2-iodoethoxy)prop-1-yne (1.707 g, 8.13 mmol) in acetone (3 mL) was added and then the reaction mixture was stirred at 60° C. for 16 hours. As the conversion was low, cesium carbonate (2.62 g, 8.05 mmol) was added and the reaction mixture which was then diluted with N,N-dimethylformamide (5 mL) and stirred at 60° C. for 68 h. The solvents were removed under reduced pressure and the resulting slurry was suspended in ethyl acetate (80 mL) and washed with water (40 mL). The aqueous layer was extracted with ethyl acetate (20 mL) and the combined organic layers were washed with brine (40 mL), dried over Na₂SO₄, filtered, and evaporated under reduced pressure. The residue was purified by flash column chromatography (silica, 0 to 7% acetone in dichloromethane) to give (4-(2-(prop-2-yn-1-yloxy)ethoxy)phenyl)methanol 132 (1.38 g) as a thick yellow oil.

LCMS: 99%, RT=1.67 min, (M−OH⁻)⁺=189 (MC Method B). ¹H NMR (400 MHz, Chloroform-d) δ 7.31-7.26 (m, 2H), 6.95-6.88 (m, 2H), 4.62 (d, J=5.5 Hz, 2H), 4.28 (d, J=2.4 Hz, 2H), 4.18-4.13 (m, 2H), 3.94-3.88 (m, 2H), 2.46 (t, J=2.4 Hz, 1H).

Step 9: Under nitrogen atmosphere at 0° C., phosphorus tribromide (0.598 mL, 6.36 mmol) was added dropwise over 10 minutes to a solution of (4-(2-(prop-2-yn-1-yloxy)ethoxy)phenyl)methanol (1.25 g, 6.06 mmol) in dichloromethane (18 mL). After 10 minutes, the reaction mixture was allowed to warm to room temperature and stirred for a further 2.5 hours. Next, the reaction mixture was diluted with dichloromethane (20 mL), cooled to 0° C., and water (4 mL) was added dropwise to the vigorously stirred reaction mixture. Then, the reaction mixture was diluted with water (16 mL) and the layers were separated. The organic layer was dried over Na₂SO₄, filtered, and evaporated under reduced pressure. The residue was purified by flash column chromatography (silica, 0 to 20% ethyl acetate in heptane) to give 1-(bromomethyl)-4-(2-(prop-2-yn-1-yloxy)ethoxy)benzene 135 (0.68 g) as a pale yellow oil.

LCMS: product reacts on column: 87%, RT=1.67 min, (M−Br⁻)⁺=189, 13%, RT=2.08 min, (M−Br⁻)⁺=189 (MC Method A). ¹H NMR (400 MHz, Chloroform-d) δ 7.35-7.28 (m, 2H), 6.92-6.84 (m, 2H), 4.50 (s, 2H), 4.27 (d, J=2.3 Hz, 2H), 4.18-4.14 (m, 2H), 3.93-3.87 (m, 2H), 2.46 (t, J=2.4 Hz, 1H).

Step 10: Under nitrogen atmosphere, cesium carbonate (756 mg, 2.321 mmol) was added to a solution of (quinuclidin-4-ylmethyl (S)-1-(4-hydroxyphenyl)-3,4-dihydroisoquinoline-2(1H)-carboxylate)trihydroborate 134 (786 mg, 1.934 mmol) in N,N-dimethylformamide (dry, 18 mL). The resulting suspension was stirred for 5 minutes after which a solution of 1-(bromomethyl)-4-(2-(prop-2-yn-1-yloxy)ethoxy)benzene 135 (572 mg, 2.127 mmol) in N,N-dimethylformamide (dry, 2 mL) was added. The reaction mixture was stirred for 2 hours and over the course of the reaction additional 1-(bromomethyl)-4-(2-(prop-2-yn-1-yloxy)ethoxy)benzene (25 mg, 0.093 mmol) was added. Then, N,N-dimethylformamide was removed under reduced pressure and the resulting amorphous solid was suspended in chloroform (60 mL), washed with brine (30 mL), and the aqueous layer was extracted with chloroform (30 mL). The combined organic layers were dried over Na₂SO₄, filtered, and concentrated under reduced pressure. The residue was purified by flash column chromatography (silica, 0 to 50% ethyl acetate in heptane) to give (quinuclidin-4-ylmethyl (S)-1-(4-((4-(2-(prop-2-yn-1-yloxy)ethoxy)benzyl)oxy)phenyl)-3,4-dihydroisoquinoline-2(1H)-carboxylate)trihydroborate 136 (1.08 g) as a white foam.

LCMS: 98%, RT=2.34 min, (M+Na)⁺=617 (MC Method A). ¹H NMR (400 MHz, Chloroform-d) mixture of rotamers δ 7.36-7.29 (m, 2H), 7.25-7.15 (m, 3H), 7.15-7.03 (m, 3H), 6.96-6.90 (m, 2H), 6.90-6.83 (m, 2H), 6.50-6.00 (br s, 1H), 4.95 (s, 2H), 4.27 (d, J=2.4 Hz, 2H), 4.19-4.13 (m, 2H), 4.05-3.93 (m, 2H), 3.93-3.86 (m, 3H), 3.30 (m, 1H), 3.11-2.90 (m, 7H), 2.80 (dt, J=16.2, 4.0 Hz, 1H), 2.46 (t, J=2.4 Hz, 1H), 1.71-1.56 (m, 6H), 1.89-1.20 (br s, 3H, BH₃).

Step 11: To a solution of (quinuclidin-4-ylmethyl (S)-1-(4-((4-(2-(prop-2-yn-1-yloxy)ethoxy)benzyl)oxy)phenyl)-3,4-dihydroisoquinoline-2(1H)-carboxylate)ytihydroborate 136 (1075 mg, 1.808 mmol) in acetone (50 mL), aqueous hydrochloric acid (1 M, 2.71 mL, 2.71 mmol) was added. After stirring the solution for 7 minutes, saturated aqueous K₂CO₃ (10 mL) was added and the resulting biphasic system was stirred vigorously for 5 minutes after which the layers were separated. The aqueous layer was extracted with chloroform (2×20 mL). The combined organic layers were dried over Na₂SO₄, filtered and concentrated under reduced pressure. The residue was purified by flash column chromatography (silica, 0 to 7.5% (7 M ammonia in methanol) in chloroform) to give quinuclidin-4-ylmethyl (S)-1-(4-((4-(2-(prop-2-yn-1-yloxy)ethoxy)benzyl)oxy)phenyl)-3,4-dihydroisoquinoline-2(1H)-carboxylate compound 2125.

Compound 2125: LCMS: 99%, RT=2.94 min, (M+H)⁺=581 (MC Method C). ¹H NMR (400 MHz, DMSO-d₆) δ 7.34 (d, J=8.3 Hz, 2H), 7.26-7.20 (m, 2H), 7.20-7.15 (m, 1H), 7.15-7.11 (m, 1H), 7.10-7.01 (m, 2H), 6.99-6.88 (m, 4H), 6.32-6.04 (br s, 1H), 4.96 (s, 2H), 4.21 (d, J=2.3 Hz, 2H), 4.13-4.08 (m, 2H), 3.90-3.80 (m, 1H), 3.79-3.72 (m, 3H), 3.72-3.64 (m, 1H), 3.47 (t, J=2.3 Hz, 1H), 3.39-3.21 (m, 1H), 2.93-2.76 (m, 2H), 2.76-2.63 (m, 6H), 1.38-1.19 (m, 6H).

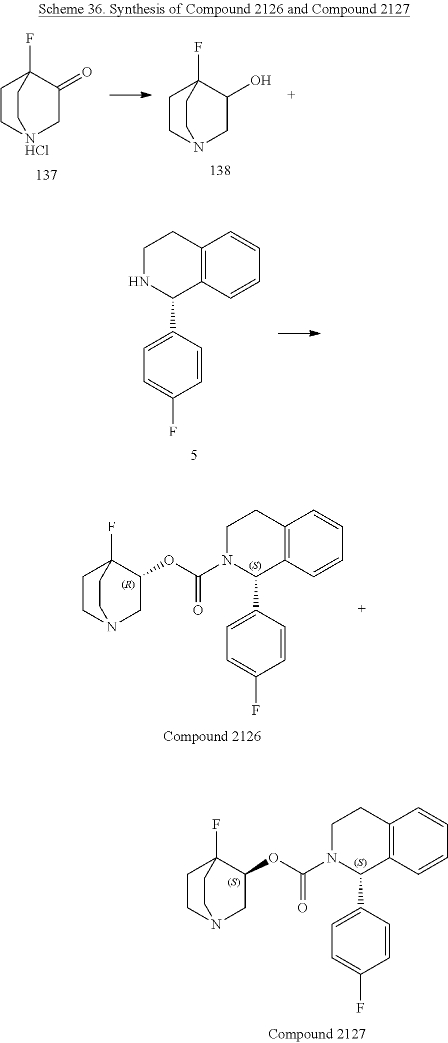

Step 1: Sodium hydroxide (21.0 mg, 0.525 mmol) was added to a solution of 4-fluoroquinuclidin-3-one hydrochloride 137 (mostly hydrate, 51.9 mg, 0.263 mmol) (ref: Bioorganic & Medicinal Chemistry Letters 23 (2013) 1684-1688) in methanol (1 mL), followed by sodium borohydride (14.9 mg, 0.394 mmol). After 2 hours, solid K₂CO₃ was added to the mixture, followed by chloroform (6 mL) and a few drops of water. The mixture was shaken, Na₂SO₄ was added, the mixture was filtered, giving a turbid filtrate. More K₂CO₃ and Na₂SO₄ was added and after stirring for 5 hours, the mixture was filtered and the filtrate was acidified with HCl in diethylether (1 M) until pH was ca. 2. The mixture was concentrated giving a colorless solid. This was dissolved in methanol, brought onto an SCX-2 column (2 g) and eluted with methanol until neutral. Next, the column was eluted with ammonia in methanol (1 M). The basic fraction was concentrated, coevaporated from chloroform-d (4 mL) and heptane (2×4 mL) giving 4-fluoroquinuclidin-3-ol 138 as a colorless solid (31 mg). ¹H NMR (400 MHz, chloroform-d) δ 3.90 (ddt, J=10.6, 8.4, 2.3 Hz, 1H), 3.24 (ddd, J=14.3, 8.5, 2.6 Hz, 1H), 3.13-2.89 (m, 4H), 2.78 (ddt, J=14.4, 5.2, 2.5 Hz, 1H), 2.24 (dtt, J=15.4, 8.0, 3.9 Hz, 1H), 1.85 (dtd, J=12.1, 9.4, 5.0 Hz, 1H), 1.72-1.47 (m, 2H), OH not visible.

Step 2: To a solution of 4-fluoroquinuclidin-3-ol 138 (31 mg, 0.214 mmol) in dry pyridine (1 mL) was added 4-nitrophenyl chloroformate (43.0 mg, 0.214 mmol). After stirring overnight, 4-nitrophenyl chloroformate (43.0 mg, 0.214 mmol) was added. A gel-like precipitate was formed. Dry pyridine (1 ml) was added. Then (S)-1-(4-fluorophenyl)-1,2,3,4-tetrahydroisoquinoline (72.8 mg, 0.320 mmol) was added, followed by diisopropylethylamine (0.112 ml, 0.641 mmol). After stirring for 3 days, the mixture was diluted with chloroform (10 mL) and washed with a half saturated aqueous K₂CO₃ solution (4 mL). The layers were separated using a phase separator and the organic layer was concentrated. The residue was purified with flash column chromatography (silica, 0 to 10% (7M ammonia in methanol) in chloroform), followed by acidic preparative MPLC (Linear Gradient: t=0 min 10% A, t=2 min 10% A, t=17 min 50% A; t=18 min 100% A; t=23 min 100%; detection: 210/254/280 nm) to obtain a solid. The solid was dissolved in methanol and brought onto a SCX-2 column (1 g) and eluted with methanol. Next, the column was eluted with ammonia in methanol (1 M). The basic fraction was concentrated and lyophilized from acetonitrile/water to give 4-fluoroquinuclidin-3-yl (1 S)-1-(4-fluorophenyl)-3,4-dihydroisoquinoline-2(1H)-carboxylate as a colorless solid. The diastereomers were separated using preparative chiral SFC. Peak 1 was desalted using an SCX-2 column (1 g) and lyophilized from acetonitrile/water giving (R)-4-fluoroquinuclidin-3-yl (S)-1-(4-fluorophenyl)-3,4-dihydroisoquinoline-2(1H)-carboxylate, Compound 2126.

Compound 2126: LCMS: 100%, RT=3.453 min. (M+H)⁺=399 (MC Method D).

¹H NMR (400 MHz, chloroform-d): mixture of rotamers δ 7.25-7.15 (m, 5H), 7.04 (d, J=7.4 Hz, 1H), 6.96 (t, J=8.7 Hz, 2H), 6.53-6.19 (m, 1H), 5.03-4.86 (m, 1H), 4.25-3.93 (m, 1H), 3.53-3.36 (m, 1H), 3.35-3.13 (m, 1H), 3.13-2.95 (m, 5H), 2.94-2.72 (m, 2H), 2.34-2.08 (m, 1H), 1.99-1.47 (m, 3H).

Chiral SFC: 100%, RT=2.821 min. (MC Method F).

Peak 2 was desalted using an SCX-2 column (1 g) and lyophilized from acetonitrile/water giving 6.7 mg of (S)-4-fluoroquinuclidin-3-yl (S)-1-(4-fluorophenyl)-3,4-dihydroisoquinoline-2(1H)-carboxylate, Compound 2127 as a colorless amorphous powder.

Compound 2127: LCMS: 100%, RT=3.451 min. (M+H)⁺=399 (MC Method D).

¹H NMR (400 MHz, chloroform-d): mixture of rotamers δ 7.26-7.14 (m, 5H), 7.10-7.04 (m, 1H), 7.04-6.90 (m, 2H), 6.51-6.10 (m, 1H), 5.02-4.85 (m, 1H), 4.20-3.85 (m, 1H), 3.53-3.27 (m, 2H), 3.18-2.67 (m, 7H), 2.30-2.06 (m, 1H), 2.00-1.42 (m, 3H).

Chiral SFC: 95.3% RT=3.138 min. (MC Method F).

Absolute stereochemistry of the two isomers is based on use of stereodefined THIQ 5 and comparison of chromatographic elution order as compared to the non-fluorinated analogs and is consistent with relative biological activity.

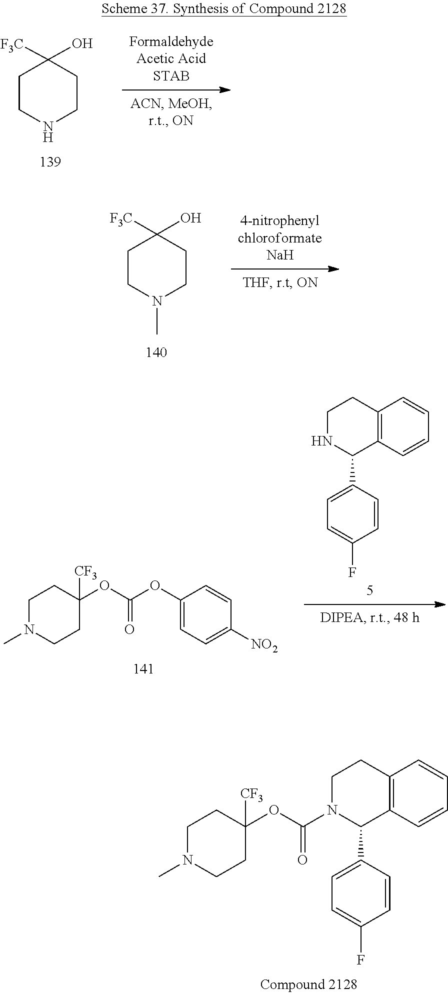 [SMILES: OC1(C(F)(F)F)CCNCC1 ; CN1CCC(O)(C(F)(F)F)CC1 ; CN1CCC(OC(=O)Oc2ccc([N+](=O)[O-])cc2)(C(F)(F)F)CC1 ; Fc1ccc([C@@H]2NCCc3ccccc32)cc1 ; CN1CCC(OC(=O)N2CCc3ccccc3[C@@H]2c2ccc(F)cc2)(C(F)(F)F)CC1]

Step 1: To a solution of 4-(trifluoromethyl)piperidin-4-ol 139 (0.25 g, 1.478 mmol) in a mixture of acetonitrile (10 mL) and methanol (10 mL), acetic acid (0.213 ml, 3.70 mmol) was added, followed by formaldehyde (37 wt % solution in water, 1.111 mL, 14.78 mmol). After stirring overnight, sodium triacetoxyborohydride (0.783 g, 3.70 mmol) was added in one batch and the reaction mixture was stirred for 1 hour. The reaction mixture was concentrated to dryness and the residue was purified by flash column chromatography (silica, 0 to 10% (7 M ammonia in methanol) in chloroform) to give 1-methyl-4-(trifluoromethyl)piperidin-4-ol 140 (0.23 g) as a white solid. GCMS1 (MC Method A20): 100%, RT=1.42 min, (M)⁺=183.1. ¹H NMR (400 MHz, Chloroform-d) δ 2.81-2.69 (m, 2H), 2.34-2.23 (m, 5H), 2.17-1.88 (m, 1H), 1.97 (td, J=13.3, 4.7 Hz, 2H), 1.70 (dq, J=14.2, 2.9 Hz, 2H).

Step 2: To a solution of 1-methyl-4-(trifluoromethyl)piperidin-4-ol 140 (0.05 g, 0.273 mmol) in anhydrous tetrahydrofuran (1 mL), sodium hydride (60% dispersion in mineral oil, 0.013 g, 0.325 mmol) was added under nitrogen atmosphere. After stirring for 30 minutes, 4-nitrophenyl chloroformate (0.055 g, 0.273 mmol) was added and stirring was continued overnight. (S)-1-(4-fluorophenyl)-1,2,3,4-tetrahydroisoquinoline 5 (0.093 g, 0.409 mmol) was added, followed by N,N-diisopropylethylamine (0.143 ml, 0.819 mmol). After 48 hours, the reaction mixture was diluted with dichloromethane, cooled to 0° C. and quenched with water. The layers were separated and the aqueous phase was extracted with dichloromethane. The combined organic layers were dried over Na₂SO₄, filtered, and concentrated under reduced pressure. The residue was purified by basic preparative MPLC (Linear Gradient: t=0 min 5% B, t=1 min 5% B, t=2 min 30% B; t=17 min 70% B; t=18 min 100% B; t=23 min 100% B; detection: 220 nm) and the product fractions were combined and lyophilized to afford 1-methyl-4-(trifluoromethyl)piperidin-4-yl (S)-1-(4-fluorophenyl)-3,4-dihydroisoquinoline-2(1H)-carboxylate Compound 2128.

Compound 2128: LCMS: 99.2%, RT=2.78 min., (M+H)⁺=437 (MC Method C).

¹H NMR (400 MHz, Chloroform-d) mixture of rotamers δ 7.20 (m, 5H), 7.11-7.04 (m, 1H), 6.97 (t, J=8.3 Hz, 2H), 6.46-6.16 (m, 1H), 4.06-3.94 (m, 1H), 3.38-3.24 (m, 1H), 3.05-2.92 (m, 1H), 2.84-2.53 (m, 5H), 2.34-2.05 (m, 4.6H), 2.01-1.75 (m 2.4H).

The following compound was prepared analogously starting with 1,4-dimethylpiperidin-4-ol:

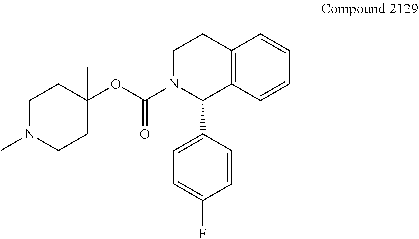 [SMILES: CN1CCC(C)(OC(=O)N2CCc3ccccc3[C@@H]2c2ccc(F)cc2)CC1]

Compound 2129: LCMS: 97.5%, RT=2.63 min., (M+H)⁺=383 (MC Method C).

¹H NMR (400 MHz, Chloroform-d+D₂O) mixture of rotamers δ 7.25-7.14 (m, 5H), 7.07 (d, J=7.1 Hz, 1H), 6.96 (t, J=8.5 Hz, 2H), 6.50-6.13 (m, 1H), 4.10-3.91 (m, 1H), 3.32-3.17 (m, 1H), 3.04-2.91 (m, 1H), 2.82-2.70 (m, 1H), 2.65-2.41 (m, 2H), 2.37-2.08 (m, 7H), 1.81-1.64 (m, 2H), 1.53 (s, 3H).

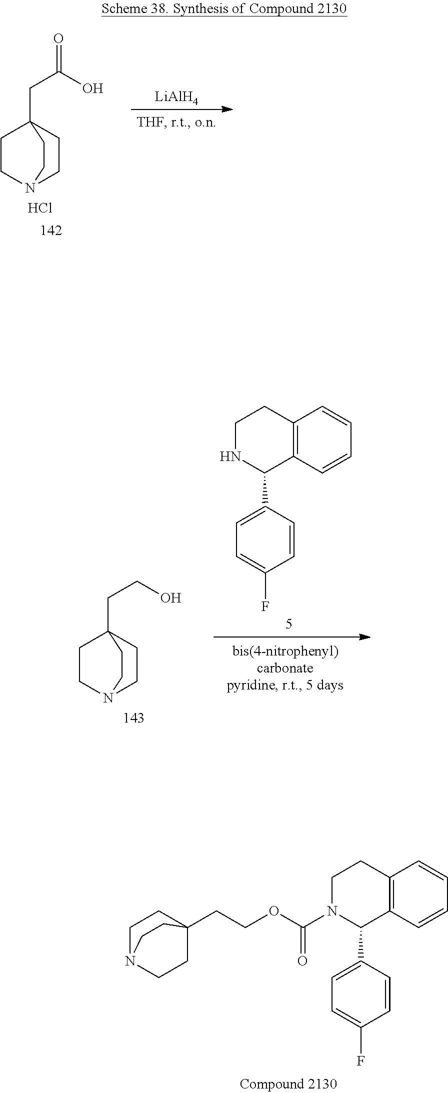 [SMILES: O=C(O)CC12CCN(CC1)CC2 ; "LiAlH4" ; Cl ; OCCC12CCN(CC1)CC2 ; Fc1ccc([C@@H]2NCCc3ccccc32)cc1 ; O=C(OCCC12CCN(CC1)CC2)N1CCc2ccccc2[C@@H]1c1ccc(F)cc1]

Step 1: To 2-(quinuclidin-4-yl)acetic acid hydrochloride 142 (60.8 mg, 0.295 mmol) was added a 7 M solution of ammonia in methanol. The solution was concentrated under reduced pressure and the residue was dissolved in tetrahydrofuran (1 ml). Under argon atmosphere, a solution of LiAlH₄ in THF (0.185 ml, 0.443 mmol) was added. The reaction mixture was stirred overnight at room temperature and quenched with water. The aqueous mixture was extracted 3 times with chloroform and the combined organic layers were dried over Na₂SO₄ and concentrated under reduced pressure affording crude 2-(quinuclidin-4-yl)ethan-1-ol 143 (69 mg) as a colorless oil which was used as such. LCMS: non-UV active, RT=0.18 min., (M+H)⁺=156 (MC Method A). ¹H NMR (400 MHz, Chloroform-d) δ 3.78-3.65 (m, 2H), 2.96-2.82 (m, 6H), 1.50-1.35 (m, 8H).

Step 2: Under nitrogen atmosphere, bis(p-nitrophenyl) carbonate (90 mg, 0.295 mmol) was added to a solution of 2-(quinuclidin-4-yl)ethan-1-ol 143 (45.8 mg, 0.295 mmol) in pyridine (1 ml) and the reaction mixture was stirred for 16 hours. (S)-1-(4-fluorophenyl)-1,2,3,4-tetrahydroisoquinoline 5 (67.0 mg, 0.295 mmol) was added and stirring was continued for 24 hours after which the reaction mixture was concentrated under reduced pressure. The residue was purified by flash column chromatography [silica, heptane/EtOAc (80-100%) followed by chloroform/7 M ammonia in methanol (10%)] afforded 2-(quinuclidin-4-yl)ethyl (S)-1-(4-fluorophenyl)-3,4-dihydroisoquinoline-2(1H)-carboxylate Compound 2130 after lyophilization.

Compound 2130: LCMS: 96%, RT=2.82 min., (M+H)⁺=409 (MC Method C).

¹H NMR (400 MHz, chloroform-d) mixture of rotamers δ 7.25-7.11 (m, 5H), 7.06-6.91 (m, 3H), 6.50-6.12 (m, 1H), 4.27-3.89 (m, 3H), 3.21 (ddd, J=13.2, 10.8, 4.4 Hz, 1H), 3.09-2.82 (m, 7H), 2.76 (dt, J=16.2, 4.0 Hz, 1H), 1.60-1.48 (m, 2H), 1.45-1.35 (m, 6H).

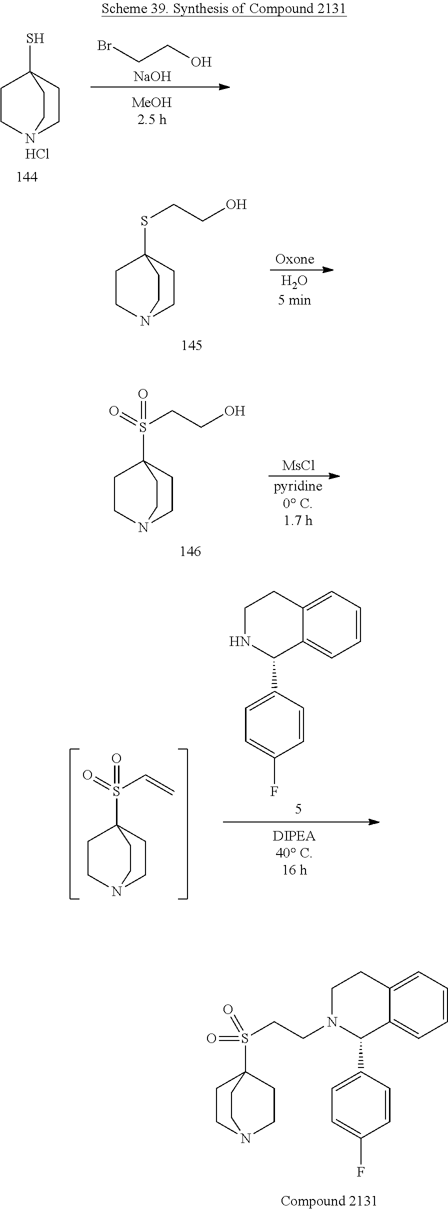

Step 1: Quinuclidine-4-thiol hydrochloride 144 (200 mg, 1.113 mmol) was added to a solution of sodium hydroxide (98 mg, 2.45 mmol) in methanol (3 mL) followed by 2-bromo-ethanol (0.079 ml, 1.113 mmol). After 2.5 hours, a few drops of water, chloroform, and solid NaHCO₃ was added and the mixture was stirred for 15 minutes. Then, Na₂SO₄ was added. The mixture was filtered and the filtrate was concentrated. The residue was purified by flash column chromatography (silica, 0 to 20% (3.5 M NH₃ in methanol) in chloroform) to give 2-(quinuclidin-4-ylthio)ethan-1-ol 145 (143 mg) as a colorless solid. LCMS: 100%, RT=0.431 min. (M+H)⁺=188 (MC Method B). ¹H NMR (400 MHz, Chloroform-d) δ 3.72 (t, J=6.2 Hz, 2H), 3.04-2.90 (m, 6H), 2.78 (t, J=6.2 Hz, 2H), 1.83-1.65 (m, 7H).

Step 2: At 0° C., a solution of Oxone (939 mg, 1.527 mmol) in water (10 mL) was added dropwise to a solution of 2-(quinuclidin-4-ylthio)ethan-1-ol 145 (143 mg, 0.763 mmol) in water (15 mL) in 5 minutes. After stirring for 5 minutes, dimethylsulfoxide (0.108 ml, 1.527 mmol) was added, followed by K₂CO₃ (317 mg, 2.290 mmol). The mixture was brought onto an ionic exchange resin column (Dowex X50W8, 200-400 MESH, 20 mL) and the column was eluted with water until neutral. Then, the column was eluted with aqeuous NH₄OH (1 M). The basic elutes (250 mL) were combined and concentrated to about 200 mL and lyophilized to give 2-(quinuclidin-4-ylsulfonyl)ethan-1-ol 146 as a ca. 1:2 mixture with 4-((2-hydroxyethyl)sulfonyl)quinuclidine 1-oxide (140 mg, ˜30% pure) as a white powder. ¹H NMR (400 MHz, Deuterium Oxide) δ 4.07-3.98 (m, 2H), 3.31-3.26 (m, 2H), 2.97-2.87 (m, 6H), 1.92-1.84 (m, 6H).

Step 3: At 0° C., Mesylchloride (0.061 mL, 0.787 mmol) was added to a suspension of 2-(quinuclidin-4-ylsulfonyl)ethan-1-ol 146 (ca. 1:2 mixture with 4-((2-hydroxyethyl)sulfonyl)quinuclidine 1-oxide, 115 mg, ˜30% pure, 0.157 mmol) in dry pyridine (4 mL). After stirring the heterogeneous mixture for 1.75 hour, (S)-1-(4-fluorophenyl)-1,2,3,4-tetrahydroisoquinoline 5 (179 mg, 0.787 mmol) was added and the mixture was allowed to warm to room temperature. Then, N,N-diisopropylethylamine (0.183 ml, 1.049 mmol) was added and the mixture was stirred at 40° C. for 16 hours. After cooling to room temperature, the mixture was diluted with chloroform (20 mL) and extracted with an ice-cold half saturated aqueous K₂CO₃ solution. The water layer was extracted with chloroform (2×5 mL) and the combined organic layers were concentrated. The residue was purified with flash column chromatography (silica, 0 to 10% (7 M NH₃ in methanol) in chloroform) followed by basic preparative MPLC (Linear Gradient: t=0 min 20% A, t=2 min 20% A, t=20 min 60% A; t=21 min 100% A; t=26 min 100%; detection: 210/254 nm). The product containing fractions were combined and lyophilized to give (S)-4-((2-(1-(4-fluorophenyl)-3,4-dihydroisoquinolin-2(1H)-yl)ethyl)sulfonyl)quinuclidine, compound 2131.

Compound 2131: LCMS: 99%, RT=2.25 min., (M+H)⁺=429 (MC Method C).

¹H NMR (400 MHz, Chloroform-d) δ 7.29-7.21 (m, 2H), 7.16-7.09 (m, 2H), 7.06-6.98 (m, 3H), 6.66 (d, J=7.8 Hz, 1H), 4.62 (s, 1H), 3.19-2.71 (m, 14H), 1.82-1.69 (m, 6H).

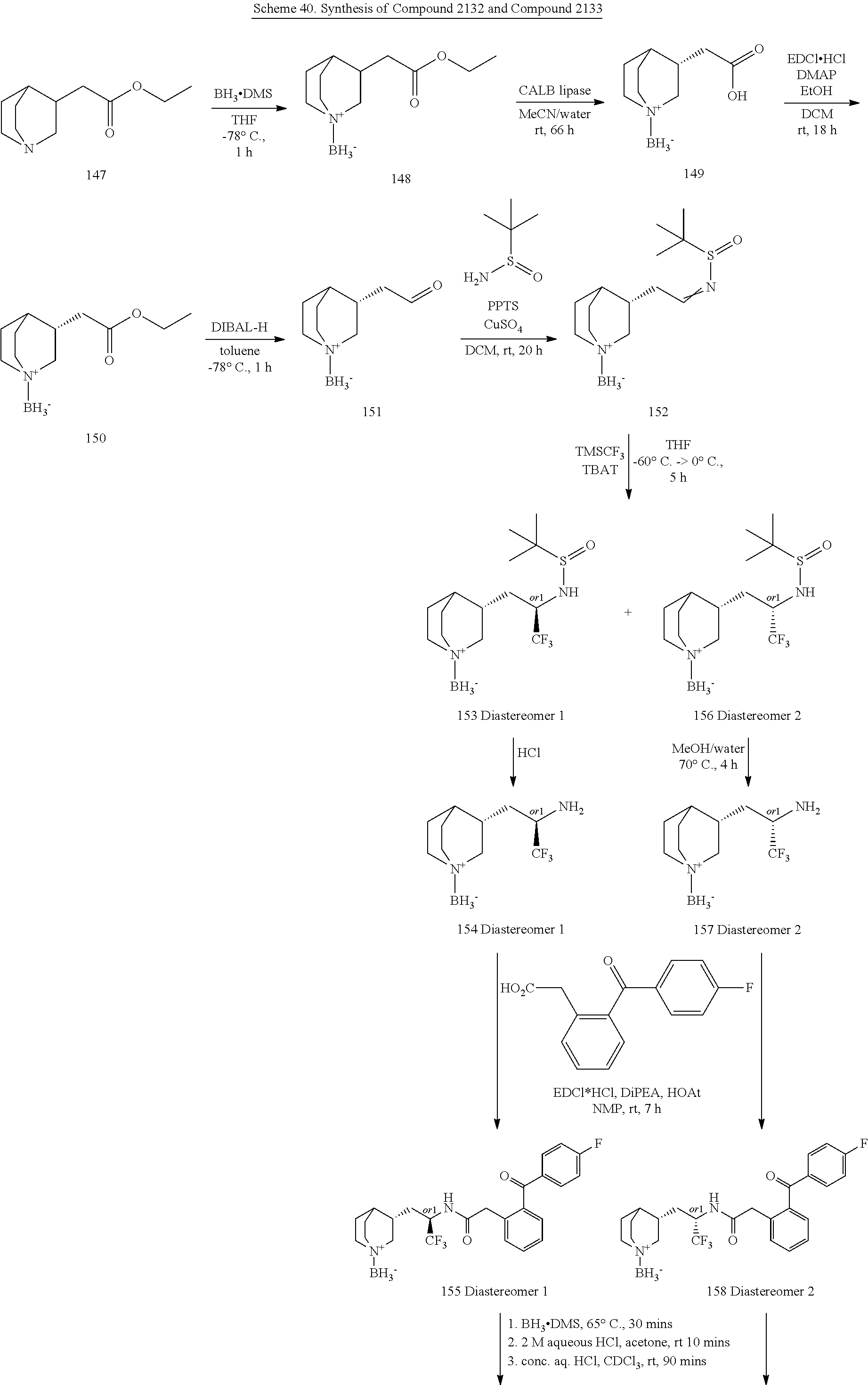

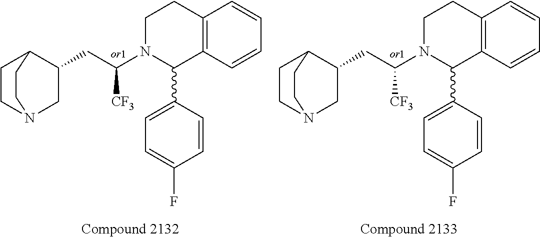

Step 1: Under a nitrogen atmosphere, ethyl 2-(quinuclidin-3-yl)acetate 147 (28.6 g, 145 mmol) was dissolved in tetrahydrofuran (270 mL) and cooled to −78° C. Borane dimethylsulfide complex (2 M in tetrahydrofuran, 72.5 mL, 145 mmol) was added slowly and the mixture was stirred at −78° C. for 1 hour. Then, the mixture was quenched with ethanol (50.8 mL, 870 mmol) at −78° C. and allowed to warm to room temperature overnight. The reaction mixture was partitioned between water (750 mL) and ethyl acetate (750 mL) and the phases were separated. The aqueous phase was extracted once more with ethyl acetate (750 mL). The combined organic phases were washed with brine (2×500 mL), dried over Na₂SO₄, and the solvent was removed under reduced pressure. The residue was purified by flash column chromatography (silica, 0 to 50% ethyl acetate in heptane) affording (3-(2-ethoxy-2-oxoethyl)quinuclidin-1-ium-1-yl)trihydroborate 148 (27.56 g) as a colorless oil. ¹H NMR (400 MHz, Chloroform-d) δ 4.14 (q, J=7.1 Hz, 2H), 3.34-3.23 (m, 1H), 3.08-2.89 (m, 4H), 2.61-2.49 (m, 1H), 2.47-2.31 (m, 3H), 1.90-1.77 (m, 4H), 1.72-1.57 (m, 1H), 1.27 (t, J=7.1 Hz, 3H), 1.75-1.00 (br s, 3H, BH₃).

Step 2: (3-(2-ethoxy-2-oxoethyl)quinuclidin-1-ium-1-yl)trihydroborate 148 (22.8 g, 108 mmol) was dissolved in a mixture of acetonitrile (150 mL) and a solution of potassium phosphate, dibasic (28.2 g, 162 mmol) and potassium phosphate, monobasic (22.05 g, 162 mmol) in water (300 ml). Lipase B from Candida antarctica (Novozym 435 (immobilised on acrylic resin, 2.3 g) was added and the mixture was shaken at room temperature for 66 hours. The mixture was filtered. The filtrate was diluted with half saturated NaHCO₃ (300 mL) and washed with ethyl acetate (3×200 mL). The aqueous layer was carefully acidified with 2 M aqueous HCl (˜125 mL) to pH˜4 and extracted with ethyl acetate (3×200 mL). The combined organic extracts from the acidic aqueous layer were dried over Na₂SO₄ and concentrated under reduced pressure to obtain (S)-(3-(carboxymethyl)quinuclidin-1-ium-1-yl)trihydroborate 149 (7.80 g) as a clear syrup which crystallized as a white solid upon standing. ¹H NMR (400 MHz, DMSO-d6) δ 12.18 (s, 1H), 3.15-3.05 (m, 1H), 2.91-2.74 (m, 4H), 2.48-2.39 (m, 2H), 2.37-2.28 (m, 1H), 2.25-2.13 (m, 1H), 1.84-1.64 (m, 4H), 1.59-1.43 (m, 1H), 1.7-1.0 (br s, 3H, BH₃).

Chiral purity determination: (S)-(3-(carboxymethyl)quinuclidin-1-ium-1-yl)trihydroborate (50 mg, 0.273 mmol), N,N-diisopropylethylamine (0.052 ml, 0.300 mmol) and (1-[bis(dimethylamino)methylene]-1H-1,2,3-triazolo[4,5-b]pyridinium 3-oxid hexafluorophosphate (104 mg, 0.273 mmol) were dissolved in N,N-dimethylformamide (1 mL). Aniline (0.025 ml, 0.273 mmol) was added and the mixture was stirred at room temperature for 1 hour. The mixture was partitioned between ethyl acetate (5 mL) and aqueous HCl (0.5 M, 5 mL). The organic layer was washed with saturated aqueous NaHCO₃, dried over Na₂SO₄ and concentrated under reduced pressure to obtain (S)-(N-phenyl-2-(quinuclidin-3-yl)acetamide)trihydroborate. Chiral LC: 98%, RT=10.31 min (MC Method I).

Step 3: N-Ethyl-N′-(3-dimethylaminopropyl)carbodiimide hydrochloride (3.46 g, 18.0 mmol) and 4-dimethylaminopyridine (0.200 g, 1.64 mmol) were added to a solution of (S)-(3-(carboxymethyl)quinuclidin-1-ium-1-yl)trihydroborate 149 (3.00 g, 16.4 mmol) in ethanol (2.5 mL, 42.8 mmol) and dichloromethane (50 mL). The mixture was stirred at room temperature for 18 hours. The reaction mixture was washed with water (20 mL) and brine (20 mL), dried over Na₂SO₄, and concentrated under reduced pressure. The residue was purified by flash column chromatography

(silica, 5 to 50% ethyl acetate in heptane) affording (ethyl (S)-2-(quinuclidin-3-yl)acetate)trihydroborate 150 (2.83 g) as a colourless oil. ¹H NMR (400 MHz, Chloroform-d) δ 4.14 (q, J=7.1 Hz, 2H), 3.36-3.24 (m, 1H), 3.09-2.91 (m, 4H), 2.62-2.50 (m, 1H), 2.44-2.31 (m, 3H), 1.93-1.76 (m, 4H), 1.71-1.60 (m, 1H), 1.27 (t, J=7.2 Hz, 3H), 1.75-1.00 (br s, 3H, BH₃).

Step 4: A solution of (ethyl (S)-2-(quinuclidin-3-yl)acetate)trihydroborate 150 (2.83 g, 13.4 mmol) in toluene (50 mL) was cooled to −78° C. under an argon atmosphere. Diisobutylaluminium hydride (1 M in hexanes) (17.40 ml, 17.40 mmol) was added dropwise and the mixture was stirred 1 hour. At −78° C., methanol (7.5 mL) was added followed by water (7.5 mL). The mixture was allowed to warm up to room temperature and filtered through a layer of Celite. The filtrate was concentrated under reduced pressure to obtain (S)-(2-(quinuclidin-3-yl)acetaldehyde)trihydroborate 151 (2.32 g, 93% (w/w), 12.9 mmol) as a clear oil. ¹H NMR (400 MHz, Chloroform-d) δ 9.81-9.73 (m, 1H), 3.40-3.29 (m, 1H), 3.11-2.88 (m, 5H), 2.71-2.54 (m, 2H), 2.53-2.39 (m, 2H), 1.90-1.03 (m, 7H).

Step 5: A mixture of (S)-(2-(quinuclidin-3-yl)acetaldehyde)trihydroborate 151 (1.01 g, 5.62 mmol), 2-methyl-2-propanesulfinamide (0.818 g, 6.75 mmol), copper(II) sulfate (1.795 g, 11.25 mmol) and pyridinium p-toluenesulfonate (0.141 g, 0.562 mmol) in dichloromethane (25 mL) was stirred at room temperature for 20 hours. The mixture was filtered over Celite and the filtrate was concentrated under reduced pressure. The residue was purified by flash column chromatography

(silica, 10 to 70% ethyl acetate in heptane) affording (2-methyl-N-(2-((S)-quinuclidin-3-yl)ethylidene)propane-2-sulfinamide)trihydroborate 152 (483 mg) as a colorless syrup. LCMS: 99%, RT=1.87 min., (M−H)⁻=269 (MC Method A). ¹H NMR (400 MHz, Chloroform-d) δ 8.11-7.97 (m, 1H), 3.37-3.27 (m, 1H), 3.06-2.94 (m, 4H), 2.67-2.61 (m, 2H), 2.61-2.52 (m, 1H), 2.47-2.29 (m, 1H), 1.95-1.74 (m, 4H), 1.72-1.62 (m, 1H), 1.23-1.16 (m, 9H), 1.75-1.00 (br s, 3H, BH₃).

Step 6: A solution of (2-methyl-N-(2-((S)-quinuclidin-3-yl)ethylidene)propane-2-sulfinamide)trihydroborate 152 (1.05 g, 3.89 mmol) and tetrabutylammonium difluorotriphenylsilicate (2.31 g, 4.27 mmol) in tetrahydrofuran (50 mL) was cooled to −60° C. under an argon atmosphere. To the resulting white slurry, (trifluoromethyl)trimethylsilane (0.864 mL, 5.44 mmol) in tetrahydrofuran (2 mL) was added and the mixture was stirred at −60° C. for 15 minutes and at 0° C. for 2 hours. Additional tetrabutylammonium difluorotriphenylsilicate (1.15 g, 2.14 mmol) was added and the mixture was cooled to −60° C. again. Additional (trifluoromethyl)trimethylsilane (0.432 mL, 2.72 mmol) was added and the mixture was stirred at 0° C. for 2 more hours. At 0° C., saturated aqueous NH₄Cl (25 mL) was added, followed by water (20 mL). The mixture was extracted with ethyl acetate (2×50 mL). The combined organic layer was dried over Na₂SO₄ and concentrated under reduced pressure. The residue was purified by flash column chromatography (silica, 10 to 80% ethyl acetate in heptane). The first eluting diastereomer (TLC (heptane:ethyl acetate=3:1): R_{f}=0.6) was obtained as a colorless syrup which was further purified by flash column chromatography (silica, 0 to 2.5% methanol in dichloromethane) followed by crystallization from diethyl ether/pentane affording (2-methyl-N-(1,1,1-trifluoro-3-((S)-quinuclidin-3-yl)propan-2-yl)propane-2-sulfinamide)trihydroborate (153, Diastereomer 1, 110 mg) as a white crystalline solid. The second eluting diastereomer (TLC (heptane:ethyl acetate=3:1): R_{f}=0.3) was obtained as a white solid and further purified by crystallization from ethyl acetate and heptane with a drop of methanol affording (2-methyl-N-(1,1,1-trifluoro-3-((S)-quinuclidin-3-yl)propan-2-yl)propane-2-sulfinamide)trihydroborate (156, Diastereomer 2, 175 mg) as a crystalline white solid.

153, Diastereomer 1: ¹H NMR (400 MHz, Chloroform-d) δ 3.74-3.58 (m, 1H), 3.34-3.21 (m, 1H), 3.12 (d, J=8.5 Hz, 1H), 3.09-2.93 (m, 4H), 2.52-2.45 (m, 1H), 2.39-2.25 (m, 1H), 2.01-1.65 (m, 6H), 1.57 (br s, 3H, BH₃), 1.55-1.48 (m, 1H), 1.25 (s, 9H).

156, Diastereomer 2: LCMS: non-UV active, RT=2.00 min., (M−H)⁻=339 (MC Method A). ¹H NMR (400 MHz, Chloroform-d) δ 3.64-3.48 (m, 1H), 3.44-3.27 (m, 1H), 3.09 (d, J=8.8 Hz, 1H), 3.06-2.93 (m, 4H), 2.50 (dd, J=13.3, 6.9 Hz, 1H), 2.40-2.28 (m, 1H), 1.99-1.90 (m, 1H), 1.90-1.75 (m, 4H), 1.75-1.64 (m, 1H), 1.59 (dd, J=10.8, 3.9 Hz, 1H), 1.9-1.1 (br s, 3H, BH₃), 1.25 (s, 9H).

Step 7a (154, Diastereomer 1): To a solution of (2-methyl-N-(1,1,1-trifluoro-3-((S)-quinuclidin-3-yl)propan-2-yl)propane-2-sulfinamide)trihydroborate (153, Diastereomer 1, 50 mg, 0.147 mmol) in methanol (1.5 mL) was added aqueous HCl (2 M, 1 mL, 2.00 mmol) and the resulting mixture was heated at 70° C. for 4 hours. The mixture was concentrated under reduced pressure to obtain a single diastereomer of 1,1,1-trifluoro-3-((S)-quinuclidin-3-yl)propan-2-amine dihydrochloride (154, Diastereomer 1, 50 mg, 85% (w/w), 0.144 mmol) as colorless syrup. ¹H NMR (400 MHz, Methanol-d4) δ 4.34-4.21 (m, 1H), 3.61 (ddd, J=12.7, 10.2, 2.5 Hz, 1H), 3.43-3.24 (m, 4H), 3.01 (ddd, J=12.9, 7.2, 2.2 Hz, 1H), 2.44-2.33 (m, 1H), 2.23-2.13 (m, 1H), 2.13-2.08 (m, 1H), 2.08-2.00 (m, 3H), 2.00-1.94 (m, 1H), 1.94-1.81 (m, 1H).

Step 7b (157, Diastereomer 2): 1,1,1-trifluoro-3-((S)-quinuclidin-3-yl)propan-2-amine dihydrochloride (157, Diastereomer 2, 175 mg, 90% (w/w), 0.512 mmol), as a single diastereoisomer, was prepared according to the procedure for 1,1,1-trifluoro-3-((S)-quinuclidin-3-yl)propan-2-amine dihydrochloride (154, Diastereomer 1) starting from (2-methyl-N-(1,1,1-trifluoro-3-((S)-quinuclidin-3-yl)propan-2-yl)propane-2-sulfinamide)trihydroborate (157, Diastereomer 2, 175 mg, 0.514 mmol). ¹H NMR (400 MHz, Methanol-d4) δ 4.31-4.19 (m, 1H), 3.69-3.60 (m, 1H), 3.42-3.22 (m, 4H), 2.97 (ddd, J=12.8, 7.2, 2.3 Hz, 1H), 2.47-2.33 (m, 1H), 2.22-2.13 (m, 1H), 2.13-1.95 (m, 5H), 1.95-1.83 (m, 1H).

Step 8a (155, Diastereomer 1): 1,1,1-trifluoro-3-((S)-quinuclidin-3-yl)propan-2-amine dihydrochloride (154, Diastereomer 1, 50 mg, 85% (w/w), 0.144 mmol) was dissolved in N,N-dimethylformamide (2 mL). N,N-diisopropylethylamine (0.075 ml, 0.432 mmol) was added, followed by 2-(2-(4-fluorobenzoyl)phenyl)acetic acid (44.6 mg, 0.173 mmol), N-ethyl-N′-(3-dimethylaminopropyl)carbodiimide hydrochloride (33.1 mg, 0.173 mmol), and 1-hydroxy-7-azabenzotriazole (2.00 mg, 0.014 mmol), and the resulting was stirred at room temperature for 16 hours. The mixture was diluted with half saturated aqueous K₂CO₃ (25 mL) and extracted with ethyl acetate (2×25 mL). The combined organic layer was washed with brine (10 mL), dried over Na₂SO₄, and concentrated under reduced pressure. The residue was purified by flash column chromatography (silica, 0 to 10% (7 M ammonia in methanol) in chloroform) to obtain a single diastereomer of 2-(2-(4-fluorobenzoyl)phenyl)-N-(1,1,1-trifluoro-3-((S)-quinuclidin-3-yl)propan-2-yl)acetamide (155, Diastereomer 1, 33 mg) as a syrup. LCMS: 99%, RT=1.61 min., (M+H)⁺=463 (MC Method A). ¹H NMR (400 MHz, Chloroform-d) δ 7.93-7.84 (m, 2H), 7.77 (d, J=9.7 Hz, 1H), 7.62 (d, J=7.7 Hz, 1H), 7.58-7.48 (m, 1H), 7.43-7.33 (m, 2H), 7.22-7.13 (m, 2H), 4.59-4.40 (m, 1H), 3.69-3.54 (m, 2H), 2.76-2.64 (m, 4H), 2.55-2.43 (m, 1H), 2.21-2.10 (m, 1H), 1.89-1.77 (m, 1H), 1.56-1.40 (m, 4H), 1.40-1.27 (m, 1H), 1.23-1.12 (m, 1H), 1.10-0.98 (m, 1H).

Step 8b (158, Diastereomer 2): A single diastereomer of 2-(2-(4-fluorobenzoyl)phenyl)-N-(1,1,1-trifluoro-3-((S)-quinuclidin-3-yl)propan-2-yl)acetamide (158, Diastereomer 2, 47 mg), as a thick syrup, was prepared according to the procedure for 2-(2-(4-fluorobenzoyl)phenyl)-N-(1,1,1-trifluoro-3-((S)-quinuclidin-3-yl)propan-2-yl)acetamide (155, Diastereomer 1), starting from 1,1,1-trifluoro-3-((S)-quinuclidin-3-yl)propan-2-amine dihydrochloride (157, Diastereomer 2, 175 mg, 90% (w/w), 0.512 mmol).

LCMS: 99%, RT=1.64 min., (M+H)⁺=463 (MC Method A). ¹H NMR (400 MHz, Chloroform-d) δ 7.93-7.85 (m, 2H), 7.75 (d, J=9.7 Hz, 1H), 7.63 (d, J=7.7 Hz, 1H), 7.58-7.51 (m, 1H), 7.43-7.34 (m, 2H), 7.23-7.13 (m, 2H), 4.55-4.37 (m, 1H), 3.69-3.54 (m, 2H), 2.86-2.74 (m, 1H), 2.74-2.63 (m, 3H), 2.53-2.38 (m, 1H), 2.25-2.16 (m, 1H), 1.85-1.76 (m, 1H), 1.54-1.43 (m, 2H), 1.42-1.35 (m, 1H), 1.31-1.06 (m, 4H).

Step 9a: To a solution of 2-(2-(4-fluorobenzoyl)phenyl)-N-(1,1,1-trifluoro-3-((S)-quinuclidin-3-yl)propan-2-yl)acetamide (155, Diastereomer 1, 33 mg, 0.071 mmol) in tetrahydrofuran (2 mL) was added borane dimethylsulfide complex (2 M in tetrahydrofuran, 0.200 mL, 0.400 mmol) after which the mixture was heated at 65° C. After 1 hour, additional borane dimethylsulfide complex (2 M in tetrahydrofuran, 0.250 mL, 0.500 mmol) was added and heating was continued for 2 hours. Methanol (5 mL) was added and the mixture was concentrated under reduced pressure. The residue was dissolved in acetone (2 mL), aqueous hydrochloric acid (2 M, 1.5 mL, 3.00 mmol) was added and the mixture was stirred at room temperature for 30 minutes. The mixture was concentrated under reduced pressure. The residue was dissolved in chloroform-d (2 mL) and concentrated aqueous hydrochloric acid (2 mL, 21.07 mmol) was added, after which the mixture was stirred vigorously for 2 hours. The mixture was diluted with chloroform-d (15 mL) and carefully basified with saturated aqueous K₂CO₃ (15 mL). The layers were separated over a phase separator and the organic layer was concentrated under reduced pressure. The residue was purified by acidic preparative MPLC (Linear Gradient: t=0 min 5% A, t=1 min 5% A, t=2 min 10% A; t=17 min 55% A; t=18 min 100%; t=23 min 100% A; detection: 210/254 nm) to obtain (3S)-3-(3,3,3-trifluoro-2-(1-(4-fluorophenyl)-3,4-dihydroisoquinolin-2(1H)-yl)propyl)quinuclidine (Compound 2132 (CF₃—CH—(NR)—(CH₂—(S)-quinuclidine stereocenter is absolute, unknown)), 3.2 mg) as a white amorphous solid after lyophilization.

Compound 2132: LCMS: 98%, RT=2.85 min., (M+H)⁺=433 (MC Method C). ¹H NMR (400 MHz, Chloroform-d) as a 1:1 mixture of diastereomers δ 7.35-7.18 (m, 3H), 7.16-7.05 (m, 2H), 7.04-6.96 (m, 3H), 6.65 (d, J=7.9 Hz, 0.5H), 6.56 (d, J=7.9 Hz, 0.5H), 5.26 (s, 0.5H), 4.94 (s, 0.5H), 3.45-2.97 (m, 4H), 2.97-2.70 (m, 6H), 2.39-2.22 (m, 1H), 1.95-1.36 (m, 7H).

Step 9b: To a solution of 2-(2-(4-fluorobenzoyl)phenyl)-N-(1,1,1-trifluoro-3-((S)-quinuclidin-3-yl)propan-2-yl)acetamide (158, Diastereomer 2, 43 mg, 0.093 mmol) in tetrahydrofuran (2 mL) was added borane dimethylsulfide complex (2 M in tetrahydrofuran, 0.250 mL, 0.500 mmol). The mixture was heated at 65° C. for 30 minutes. The reaction mixture was quenched with methanol (1 mL) and concentrated under reduced pressure. The residue was dissolved in acetone (2 mL), aqueous hydrochloric acid (2 M, 0.250 mL, 0.500 mmol) was added and the mixture was stirred at room temperature for 10 minutes. The mixture was concentrated under reduced pressure. The residue was purified by flash column chromatography (silica, 0 to 10% (7 M ammonia in methanol) in chloroform) to obtain (4-fluorophenyl)(2-(2-((1,1,1-trifluoro-3-((S)-quinuclidin-3-yl)propan-2-yl)amino)ethyl)phenyl)methanol (158a, Diastereomer 2, 22 mg, 50% (w/w), 0.024 mmol,) as a syrup, which was used as such in the next step. LCMS: 60%, RT=1.65 min., (M+H)⁺=451 (MC Method A). Concentrated aqueous hydrochloric acid (0.7 ml, 7.37 mmol) was added to a solution of (4-fluorophenyl)(2-(2-((1, 1,1-trifluoro-3-((S)-quinuclidin-3-yl)propan-2-yl)amino)ethyl)phenyl)methanol (158a, Diastereomer 2, 16 mg, 50% (w/w), 0.018 mmol) in chloroform (0.7 mL). the resulting biphasic system was stirred vigorously for 90 minutes. The mixture was diluted with chloroform (10 mL) and basified with half saturated K₂CO₃ (10 mL). The layers were separated over a phase separation filter and the organic layer was concentrated under reduced pressure. The mixture was purified by acidic preparative MPLC (Linear Gradient: t=0 min 5% A, t=1 min 5% A, t=2 min 10% A; t=17 min 55% A; t=18 min 100%; t=23 min 100% A; detection: 210/254 nm). Product fraction lyophilized to obtain (3S)-3-(3,3,3-trifluoro-2-(1-(4-fluorophenyl)-3,4-dihydroisoquinolin-2(1H-yl)propyl)quinuclidine (Compound 2133 (CF₃—CH—(NR)—(CH₂—(S)-quinuclidine stereocenter is absolute, unknown)), 1.0 mg) as a white amorphous solid.

Compound 2133: LCMS: 96%, RT=2.83 min., (M+H)⁺=433 (MC Method C).

¹H NMR (400 MHz, Chloroform-d) as a 5:3 mixture of diastereomers, δ 7.33-7.17 (m, 3H), 7.16-7.05 (m, 2H), 7.04-6.94 (m, 3H), 6.64 (d, J=7.8 Hz, 0.37H), 6.58 (d, J=7.8 Hz, 0.63H), 5.25 (s, 0.63H), 4.93 (s, 0.37H), 3.38-3.28 (m, 1H), 3.26-2.51 (m, 8H), 2.37-2.30 (m, 0.37H), 2.29-2.18 (m, 0.63H), 1.93-1.39 (m, 8H).

All the following compounds were prepared using General Procedure GP-4 (Compounds 2134-2143):

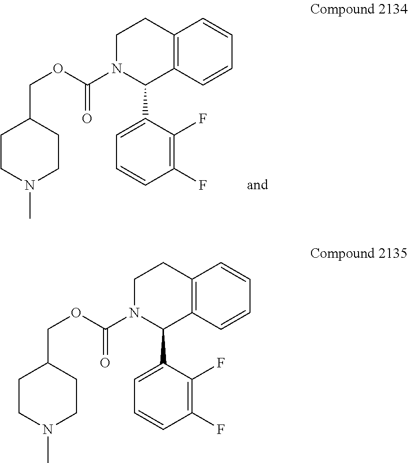 [SMILES: CN1CCC(COC(=O)N2CCc3ccccc3[C@@H]2c2cccc(F)c2F)CC1 ; CN1CCC(COC(=O)N2CCc3ccccc3[C@H]2c2cccc(F)c2F)CC1]

The diastereomers were separated by chiral SFC eluting with CO₂/MeOH containing 0.2% ammonia/methanol over an EnantioPak® AS column (4.6×100 mm 5 μm) to give Compound 2134 (retention time 2.2 min) and Compound 2135 (retention time 2.92 min). Stereochemical assignment at the 1 position of the tetrahydroisoquinoline is assigned based on chromatographic elution order as compared to diastereomers of related analogues of known configuration.

Compound 2134: LCMS: (M+H)⁺ 401; retention time 1.831 min. CP Method C

¹H NMR (400 MHz, CDCl₃) δ 7.24-7.13 (m, 3H), 7.11-6.99 (m, 2H), 6.98-6.92 (m, 1H), 6.84 (s, 1H), 6.48 (s, 1H), 4.28-4.05 (m, 1H), 4.04-3.98 (m, 1H), 3.94 (s, 1H), 3.50-3.38 (m, 1H), 3.08-2.94 (m, 1H), 2.93-2.76 (m, 3H), 2.26 (s, 3H), 1.96-1.84 (m, 2H), 1.75-1.51 (m, 3H), 1.42-1.21 (m, 2H).

Chiral SFC: EtOH (1% ammonia/methanol) over an ENANTIOPAK® IG column (4.6×100 mm, 5 μm), retention time 2.2 min).

Compound 2135: LCMS: (M+H)⁺=401; purity=100% (214 nm); retention time=1.421 min. CP Method E

¹H NMR (400 MHz, CDCl₃) δ 7.24-7.13 (m, 3H), 7.11-6.99 (m, 2H), 6.98-6.92 (m, 1H), 6.84 (s, 1H), 6.48 (s, 1H), 4.28-4.06 (m, 1H), 4.04-3.98 (m, 1H), 3.94 (s, 1H), 3.50-3.39 (m, 1H), 3.08-2.94 (m, 1H), 2.93-2.78 (m, 3H), 2.26 (s, 3H), 1.96-1.85 (m, 2H), 1.77-1.51 (m, 3H), 1.44-1.16 (m, 2H).

Chiral SFC: EtOH (1% ammonia/methanol) over an ENANTIOPAK® IG column (4.6×100 mm, 5 μm), retention time 2.92 min).

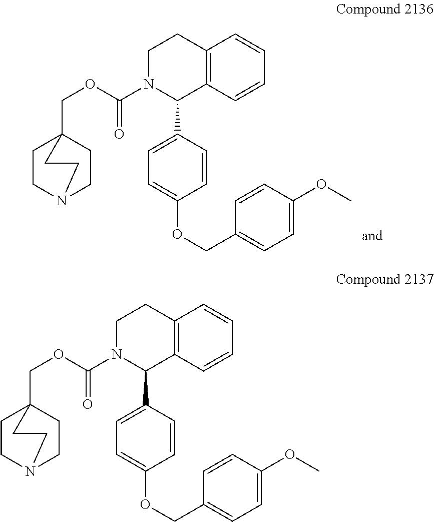 [SMILES: COc1ccc(COc2ccc([C@H]3c4ccccc4CCN3C(=O)OCC34CCN(CC3)CC4)cc2)cc1 ; COc1ccc(COc2ccc([C@@H]3c4ccccc4CCN3C(=O)OCC34CCN(CC3)CC4)cc2)cc1]

The diastereomers were separated by chiral SFC eluting with CO₂/EtOH containing 0.2% ammonia/methanol over an EnantioPak® AD column (20×250 mm, 10 μm) to give Compound 2136 (retention time 11.1 min) and Compound 2137 (retention time 8.8 min). Stereochemical assignment at the 1 position of the tetrahydroisoquinoline is assigned based on chromatographic elution order as compared to diastereomers of related analogues of known configuration.

Compound 2136: LCMS: (M+H)⁺ 513 retention time 1.544 min. CP Method A2

¹H NMR (400 MHz, CD₃OD) δ 7.32 (d, J=8.8 Hz, 2H), 7.24-7.15 (m, 3H), 7.14-7.05 (m, 3H), 6.90 (d, J=8.8 Hz, 4H), 6.25 (s, 1H), 4.96 (s, 2H), 4.01-3.90 (m, 3H), 3.78 (s, 3H), 3.44-3.34 (m, 1H), 3.18-2.98 (m, 6H), 2.94 (ddd, J=24.0, 10.0, 6.0 Hz, 1H), 2.88-2.78 (m, 1H), 1.66 (s, 6H).

Chiral SFC: n-Hexane (0.1% DEA):EtOH (0.1% DEA)=10:90 over an ENANTIOPAK® IG column (4.6×250 mm, 5 μm), retention time 23.79 min).

Compound 2137: LCMS: (M+H)⁺ 513; purity 98% (214 nm); retention time 1.545 min. CP Method A2

¹H NMR (400 MHz, CD₃OD) δ 7.34 (d, J=8.4 Hz, 2H), 7.25-7.17 (m, 3H), 7.15-7.06 (m, 3H), 6.92 (d, J=8.8 Hz, 4H), 6.26 (s, 1H), 4.99 (s, 2H), 3.98 (dt, J=10.4, 4.2 Hz, 1H), 3.88 (q, J=6.4 Hz, 1H), 3.81 (s, 3H), 3.44-3.34 (m, 1H), 3.00-2.80 (m, 8H), 1.51 (s, 6H).

Chiral SFC: n-Hexane (0.1% DEA):EtOH (0.1% DEA)=10:90 over an ENANTIOPAK® IG column (4.6×250 mm, 5 μm), retention time 33.751 min).

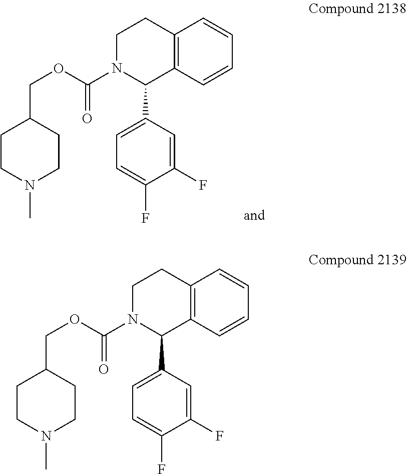 [SMILES: CN1CCC(COC(=O)N2CCc3ccccc3[C@@H]2c2ccc(F)c(F)c2)CC1 ; CN1CCC(COC(=O)N2CCc3ccccc3[C@H]2c2ccc(F)c(F)c2)CC1]

The diastereomers were separated by chiral SFC eluting with MeOH (0.2% ammonia/methanol) over an EnantioPak® AD (4.6×100 mm, 5 μm) to give Compound 2138 (retention time=2.31 min) and Compound 2139 (retention time=1.23 min). Stereochemical assignment at the 1 position of the (1-methylpiperidin-4-yl)methyl-1-(3,4-difluorophenyl)-3,4-dihydroisoquinoline-2(1H)-carboxylate is assigned based on chromatographic elution order as compared to diastereomers of related analogues of known configuration.

Compound 2138: LCMS: (M+H)⁺ 401.2; purity 100% (214 nm); retention time 1.456 min. CP Method C

¹H NMR (400 MHz, CDCl₃) δ 7.28-7.23 (m, 3H), 7.10-7.04 (m, 3H), 6.96 (s, 1H), 6.39-6.23 (m, 1H), 4.09-4.00 (m, 3H), 3.26-3.20 (m, 1H), 2.94 (d, J=11.6 Hz, 3H), 2.77 (d, J=16 Hz, 1H), 2.32 (s, 3H), 2.03-1.98 (m, 2H), 1.73 (d, J=11.6 Hz, 3H), 1.49-1.43 (m, 2H).

Chiral SFC: EtOH (1% ammonia/methanol) over an ENANTIOPAK® IG column (4.6×100 mm 5 μm), retention time 1.85 min).

Compound 2139: LCMS: (M+H)⁺ 401.2; purity 100% (214 nm); retention time 1.456 min. CP Method C

¹H NMR (400 MHz, CDCl₃) δ 7.28-7.19 (m, 3H), 7.10-7.04 (m, 3H), 6.96 (s, 1H), 6.39-6.22 (m, 1H), 4.09-4.00 (m, 3H), 3.23-3.18 (m, 1H), 2.88 (d, J=11.2 Hz, 3H), 2.77 (d, J=16 Hz, 1H), 2.28 (s, 3H), 1.96-1.88 (m, 3H), 1.73-1.70 (m, 3H), 1.44-1.37 (m, 2H).

Chiral SFC: EtOH (1% ammonia/methanol) over an ENANTIOPAK® IG column (4.6×100 mm 5 μm), retention time 2.04 min).

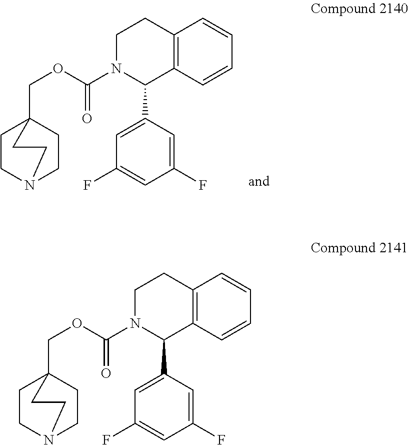 [SMILES: O=C(OCC12CCN(CC1)CC2)N1CCc2ccccc2[C@@H]1c1cc(F)cc(F)c1 ; O=C(OCC12CCN(CC1)CC2)N1CCc2ccccc2[C@H]1c1cc(F)cc(F)c1]

The diastereomers were separated by chiral SFC eluting with EtOH (0.1% ammonia/methanol) over an EnantioPak® IG (4.6×100 mm 5 μm) to give Compound 2140 (retention time=2.27 min) and Compound 2141 (retention time=3.12 min). Stereochemical assignment at the 1 position of the quinuclidin-4-ylmethyl-1-(3,5-difluorophenyl)-3,4-dihydroisoquinoline-2(1H)-carboxylate is assigned based on chromatographic elution order as compared to diastereomers of related analogues of known configuration.

Compound 2140: LCMS: (M+H)⁺ 413.3; purity 100% (214 nm); retention time 1.460 min. CP Method C

¹H NMR (400 MHz, CDCl₃) δ 7.25-7.20 (m, 3H), 7.08 (d, J=7.6 Hz, 1H), 6.75-6.67 (m, 3H), 6.37-6.15 (m, 1H), 4.07-3.86 (m, 2H), 3.83-3.80 (m, 1H), 3.34-3.27 (m, 1H), 2.89 (t, J=7.6 Hz, 7H), 2.79-2.75 (m, 1H), 1.41 (s, 6H).

Chiral SFC: EtOH (1% ammonia/methanol) over an ENANTIOPAK® IG column (4.6×100 mm 5 μm), retention time 2.24 min).

Compound 2141: LCMS: (M+H)⁺ 413.3; purity 100% (214 nm); retention time 1.459 min. CP Method C

¹H NMR (400 MHz, CDCl₃) δ 7.27-7.22 (m, 3H), 7.11 (d, J=7.6 Hz, 1H), 6.78-6.71 (m, 3H), 6.40-6.18 (m, 1H), 4.09-3.88 (m, 1H), 3.85-3.83 (m, 2H), 3.69-3.30 (m, 1H), 2.91 (t, J=7.6 Hz, 7H), 2.82-2.78 (m, 1H), 1.43 (s, 6H).

Chiral SFC: EtOH (1% ammonia/methanol) over an ENANTIOPAK® IG column (4.6×100 mm 5 μm), retention time 3.17 min).

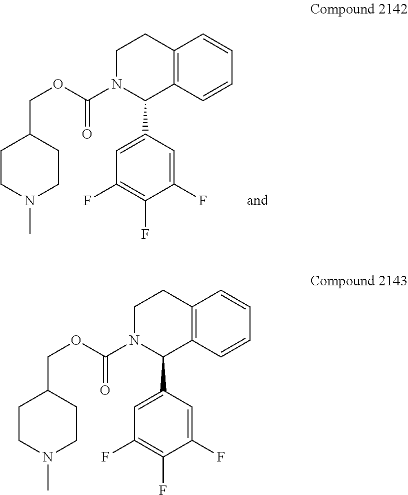 [SMILES: CN1CCC(COC(=O)N2CCc3ccccc3[C@@H]2c2cc(F)c(F)c(F)c2)CC1 ; CN1CCC(COC(=O)N2CCc3ccccc3[C@H]2c2cc(F)c(F)c(F)c2)CC1]

The diastereomers were separated by chiral SFC eluting with EtOH containing 1% ammonia/methanol over an EnantioPak® AD-H column (4.6×100 mm 5 μm) to give Compound 2142 (retention time 1.57 min) and Compound 2143 (retention time 0.97 min). Stereochemical assignment at the 1 position of the tetrahydroisoquinoline is assigned based on chromatographic elution order as compared to diastereomers of related analogues of known configuration.

Compound 2142: LCMS: (M+H)⁺ 419; purity 100% (214 nm); Retention time 2.026 min. CP Method C

¹H NMR (400 MHz, DMSO-d₆) δ 7.83 (s, 1H), 7.77-7.69 (m, 1H), 7.43 (d, J=8.2 Hz, 1H), 7.18-7.13 (m, 3H), 6.59 (s, 1H), 6.42 (d, J=5.9 Hz, 1H), 3.96-3.84 (m, 1H), 3.74-3.64 (br, s, 1H), 3.26-3.19 (m, 4H), 3.12-2.93 (m, 3H), 2.89-2.77 (m, 2H), 2.73-2.57 (m, 3H), 1.82-1.73 (m, 2H), 1.55 (d, J=6.6 Hz, 2H), 1.30 (s, 1H).

Chiral SFC: EtOH (1% ammonia/methanol) over an ENANTIOPAK® IG column (4.6*100 mm 5 μm), retention time=1.7 min).

Compound 2143: LCMS: (M+H)⁺ 419; purity 100% (214 nm); Retention time 2.028 min. CP Method C

¹H NMR (400 MHz, CD₃OD) δ 7.21-7.09 (m, 3H), 7.02 (d, J=7.3 Hz, 1H), 6.90-6.78 (m, 2H), 6.17 (s, 1H), 5.39 (s, 1H), 4.00-3.90 (m, 2H), 3.85 (dt, J=13.1, 5.3 Hz, 1H), 3.35-3.24 (m, 1H), 2.89-2.66 (m, 4H), 2.17 (s, 3H), 1.92 (t, J=12.0 Hz, 2H), 1.59 (d, J=11.8 Hz, 3H), 1.37-1.20 (m, 2H).

Chiral SFC: EtOH (1% ammonia/methanol) over an ENANTIOPAK® IG column (4.6×100 mm 5 μm), retention time 1.87 min).

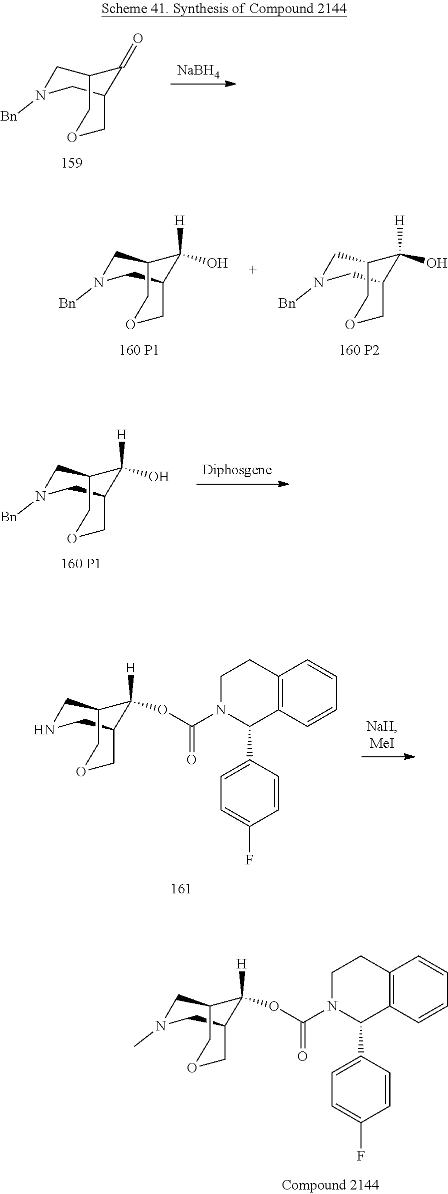

Step 1: To a mixture of (1R, 5S)-7-benzyl-3-oxa-7-azabicyclo [3.3.1] nonan-9-one 159 (1.5 g, 6.49 mmol) dissolved in MeOH (30 mL) was added NaBH₄ (741 mg, 19.5 mmol) slowly at 0° C. The reaction mixture was stirred at 0° C. for 2 hours. The reaction mixture was concentrated and diluted with water (30 mL), extracted by three 30 mL portions of ethyl acetate. The combined organic phases were washed by brine (60 mL), dried and concentrated to obtain a crude solid, which was purified by prep-HPLC to afford 450 mgs of (1R, 5S, 9r)-7-benzyl-3-oxa-7-azabicyclo[3.3.1]nonan-9-ol 160 P1 and its isomer, 160 P2

LCMS: retention time=1.13 min, (M+H)⁺=233, purity: 100% (214 nm)

Step 2: To a mixture of (1R, 5S, 9r)-7-benzyl-3-oxa-7-azabicyclo[3.3.1]nonan-9-ol (233 mg, 1 mmol) 160 P1 dissolved in CH₃CN (5 mL) was added diphosgene (119 mg, 0.6 mmol) at 0° C. The resulting mixture was stirred at 25° C. for 1 hr. The mixture was concentrated to obtain a light yellow solid, which was added into another mixture of (S)-1-(4-fluorophenyl)-1, 2, 3, 4-tetrahydroisoquinoline 5 (227 mg, 1 mmol) and TEA (303 mg, 3 mmol) dissolved in DMF (10 mL). The reaction mixture was stirred at 60° C. for 16 hours. The reaction mixture was diluted to water (50 mL), extracted by three 20 mL portions of ethyl acetate. The combined organic phases were washed by brine (50 mL*3), dried over anhydrous Na₂SO₄, filtered and concentrated to obtain a crude solid, which was purified by prep-HPLC to afford (S)-((1R,5S,9r)-3-oxa-7-azabicyclo[3.3.1]nonan-9-yl) 1-(4-fluorophenyl)-3,4-dihydroisoquinoline-2(1H)-carboxylate 161 (70 mg).

LCMS: retention time=1.51 min, (M+H)⁺=397, purity: 100% (214 nm).

Step 3 (S)-((1R, 5S, 9r)-3-oxa-7-azabicyclo [3.3.1] nonan-9-yl) 1-(4-fluorophenyl)-3, 4-dihydroisoquinoline-2(1H)-carboxylate 161 (70 mg, 0.18 mmol) in DMF (2 mL) was added NaH (8 mg, 0.2 mmol) at 0° C. Mel (25 mg, 0.18 mmol) was added, then the mixture was stirred at room temperature for 0.5 h. The reaction mixture was diluted with water (20 mL) and extracted with three 10 mL portions of ethyl acetate. The combined organic phases were washed by brine (20 mL), dried over anhydrous Na₂SO₄, filtered and concentrated to obtain a crude oil, which was purified by prep-HPLC to afford (S)-((1R,5S,9r)-7-methyl-3-oxa-7-azabicyclo[3.3.1]nonan-9-yl) 1-(4-fluorophenyl)-3,4-dihydroisoquinoline-2(1H)-carboxylate, compound 2144.

Compound 2144: LCMS: (M+H)⁺=411, purity=100% (214 nm), Retention time=1.388 min. CP Method A1

¹H NMR (400 MHz, CDCl₃) δ 7.26-7.15 (m, 5H), 7.08 (s, 1H), 6.98 (t, J=8.5 Hz, 2H), 6.43 (s, 1H), 4.96 (t, J=3.3 Hz, 1H), 4.09 (dd, J=23.5, 12.6 Hz, 3H), 3.84 (dd, J=11.6, 2.2 Hz, 2H), 3.32 (s, 1H), 3.09-2.89 (m, 3H), 2.82 (dt, J=16.3, 4.1 Hz, 1H), 2.55 (t, J=11.6 Hz, 2H), 2.28 (s, 3H), 1.98 (d, J=20.5 Hz, 2H).

All the following compounds were prepared using General Procedure GP-4 (Compounds 2145-2150):

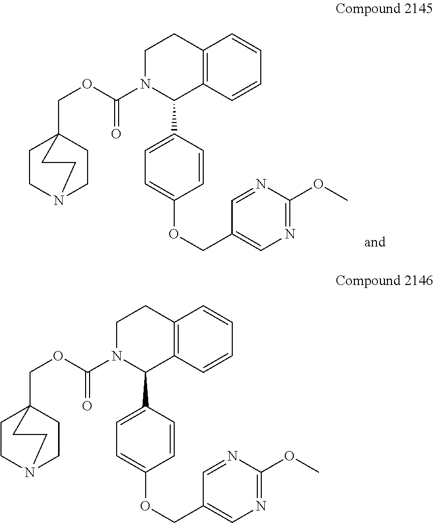 [SMILES: COc1ncc(COc2ccc([C@H]3c4ccccc4CCN3C(=O)OCC34CCN(CC3)CC4)cc2)cn1 ; COc1ncc(COc2ccc([C@@H]3c4ccccc4CCN3C(=O)OCC34CCN(CC3)CC4)cc2)cn1]

The diastereomers were separated by chiral SFC eluting with CO₂/EtOH containing 0.5% ammonia/methanol over an EnantioPak® AD column (20×250 mm 10 μm) to give Compound 2145 (retention time 4.03 min) and Compound 2146 (retention time 1.74 min). Stereochemical assignment at the 1 position of the tetrahydroisoquinoline is based on chromatographic elution order as compared to diastereomers of related analogues of known configuration.

Compound 2145: LCMS: (M+H)⁺ 515; purity 97% (214 nm); retention time=1.713 min. CP Method B

¹H NMR (400 MHz, CD₃OD) δ 8.64 (s, 2H), 7.25-7.22 (m, 2H), 7.21-7.13 (m, 3H), 7.09 (s, 1H), 6.96 (d, J=8.0 Hz, 2H), 6.29 (s, 1H), 5.04 (s, 2H), 4.02 (s, 3H), 4.01-3.94 (m, 1H), 3.92-3.83 (m, 2H), 3.37 (s, 1H), 3.00-2.89 (m, 8H), 1.51 (s, 6H).

Chiral SFC: n-Hexane (0.1% DEA):EtOH (0.1% DEA)=10:90 over an ENANTIOPAK® IG column (4.6×250 mm 5 μm), retention time 34.36 min).

Compound 2146: LCMS: (M+H)⁺ 515; purity 100% (214 nm); retention time 1.717 min. CP Method B

¹H NMR (400 MHz, CD₃OD) δ 8.62 (s, 2H), 7.24-7.20 (m, 2H), 7.20-7.12 (m, 3H), 7.07 (s, 1H), 6.95 (d, J=8.0 Hz, 2H), 6.26 (s, 1H), 5.03 (s, 2H), 4.01 (s, 3H), 3.96 (dt, J=13.2, 4.8 Hz, 1H), 3.90-3.81 (m, 2H), 3.35 (s, 1H), 3.00-2.76 (m, 8H), 1.49 (s, 6H).

Chiral SFC: n-Hexane (0.1% DEA):EtOH (0.1% DEA)=10:90 over an ENANTIOPAK® IG column (4.6×250 mm 5 μm), retention time 42.53 min).

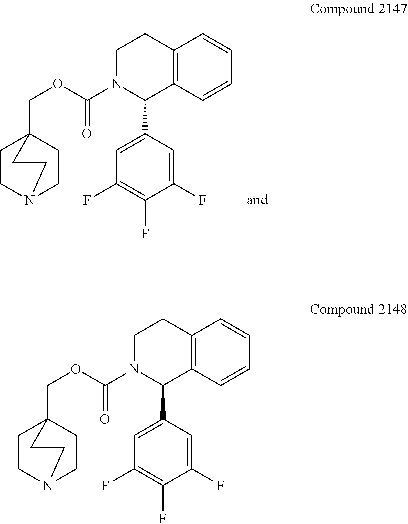 [SMILES: O=C(OCC12CCN(CC1)CC2)N1CCc2ccccc2[C@@H]1c1cc(F)c(F)c(F)c1 ; O=C(OCC12CCN(CC1)CC2)N1CCc2ccccc2[C@H]1c1cc(F)c(F)c(F)c1]

The diastereomers were separated by chiral SFC eluting with EtOH (1% ammonia/methanol) over an EnantioPak® IG column (4.6×100 mm 5 μm) to give Compound 2147 (retention time 1.47 min) and Compound 2148 (retention time 2.18 min). Stereochemical assignment at the 1 position of the tetrahydroisoquinoline is assigned on chromatographic elution order as compared to diastereomers of related analogues of known configuration.

Compound 2147: LCMS: (M+H)⁺ 431; purity 96.9% (214 nm); Retention time 1.886 min. CP Method C

¹H NMR (400 MHz, CD₃OD) δ 7.32-7.21 (m, 3H), 7.16 (s, 1H), 6.96 (s, 2H), 6.27 (s, 1H), 3.99-3.86 (m, 3H), 3.51-3.36 (m, 1H), 3.07-2.89 (m, 7H), 2.88-2.79 (m, 1H)

Chiral SFC: EtOH (1% ammonia/methanol) over an ENANTIOPAK® IG column (4.6×100 mm 5 μm), retention time 1.53 min).

Compound 2148: LCMS: (M+H)⁺ 431; purity 95.6% (214 nm); Retention time 1.859 min. CP Method C

¹H NMR (400 MHz, CD₃OD) δ 8.55 (s, 1H), 7.33-7.22 (m, 3H), 7.15 (s, 1H), 6.96 (s, 2H), 6.28 (s, 1H), 4.03-3.85 (m, 3H), 3.53-3.35 (br, s, 1H), 3.18-3.01 (br, s, 6H), 2.95 (ddd, J=14.9, 9.0, 5.6 Hz, 1H), 2.83 (d, J=16.2 Hz, 1H), 1.75-1.51 (br, s, 6H).

Chiral SFC: EtOH (1% ammonia/methanol) over an ENANTIOPAK® IG column (4.6×100 mm 5 μm), retention time 2.18 min).

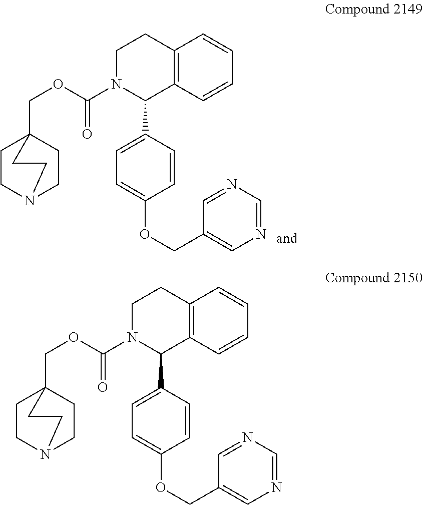

The diastereomers were separated by chiral SFC eluting with CO₂/EtOH containing 0.5% ammonia/methanol over an EnantioPak® AD column (20×250 mm 10 μm) to give Compound 2149 (retention time=4.63 min) and Compound 2150 (retention time=2.18 min). Stereochemical assignment at the 1 position of the tetrahydroisoquinoline is assigned based on chromatographic elution order as compared to diastereomers of related analogues of known configuration.

Compound 2149: LCMS: (M+H)⁺ 485; purity 100% (214 nm); retention time 1.627 min. CP Method B

¹H NMR (400 MHz, CDCl₃) δ 9.22 (s, 1H), 8.83 (s, 2H), 7.26-7.20 (m, 3H), 7.17 (d, J=7.6 Hz, 2H), 7.07 (d, J=6.4 Hz, 1H), 6.90 (d, J=7.6 Hz, 2H), 6.45-6.06 (br, 1H), 5.08 (s, 2H), 4.14-3.90 (m, 3H), 3.32 (s, 7H), 3.05-2.97 (m, 1H), 2.83 (dt, J=16.0, 4.0 Hz, 1H), 1.80 (s, 6H).

Chiral SFC: n-Hexane (0.1% DEA):EtOH (0.1% DEA)=10:90 over an ENANTIOPAK® IG column (4.6×250 mm 5 μm), retention time 43.29 min).

Compound 2150: LCMS: (M+H)⁺ 485; purity 100% (214 nm); retention time 1.622 min. CP Method B

¹H NMR (400 MHz, CDCl₃) δ 9.21 (s, 1H), 8.82 (s, 2H), 7.26-7.15 (m, 5H), 7.06 (d, J=6.4 Hz, 1H), 6.89 (d, J=8.8 Hz, 2H), 6.50-6.15 (br, 1H), 5.06 (s, 2H), 4.20-3.95 (m, 1H), 3.92-3.78 (m, 2H), 3.26 (ddd, J=20.0, 10.8, 4.4 Hz, 1H), 3.05-2.85 (m, 5H), 2.79 (dt, J=16.0, 3.6 Hz, 1H), 2.48 (s, 2H), 1.48-1.40 (m, 6H).

Chiral SFC: n-Hexane (0.1% DEA):EtOH (0.1% DEA)=10:90 over an ENANTIOPAK® IG column (4.6×250 mm 5 μm), retention time 47.45 min).

All the following compounds were prepared using General Procedure GP-5 (Compounds 2151-2152):

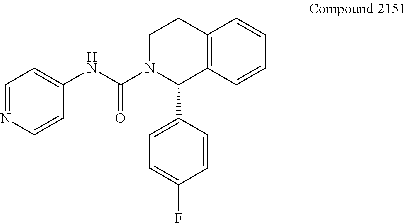 [SMILES: O=C(Nc1ccncc1)N1CCc2ccccc2[C@@H]1c1ccc(F)cc1]

Compound 2151: LCMS: (M+H)⁺ 348; purity 100% (214 nm); retention time 1.758 min. CP Method C

¹H NMR (400 MHz, DMSO-d₆) δ 9.06 (s, 1H), 8.33 (d, J=6.4 Hz, 2H), 8.15 (s, 1H), 7.57-7.51 (m, 2H), 7.31-7.25 (m, 2H), 7.24-7.19 (m, 3H), 7.18-7.11 (m, 2H), 6.58 (s, 1H), 4.05-3.95 (m, 1H), 3.45-3.35 (m, 1H), 3.04-2.93 (m, 1H), 2.85-2.75 (m, 1H).

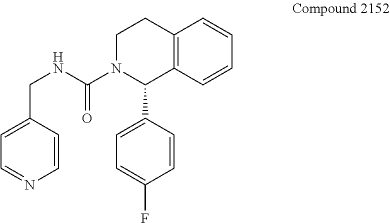 [SMILES: O=C(NCc1ccncc1)N1CCc2ccccc2[C@@H]1c1ccc(F)cc1]

Compound 2152: LCMS: (M+H)⁺ 362; purity 95% (214 nm); retention time 1.437 min. CP Method A2

¹H NMR (400 MHz, DMSO-d₆) δ 8.49-8.45 (m, 2H), 7.35 (t, J=6.0 Hz, 1H), 7.28-7.21 (m, 5H), 7.20-7.15 (m, 4H), 7.14-7.09 (m, 1H), 6.44 (s, 1H), 4.40-4.22 (m, 2H), 3.84-3.72 (m, 1H), 3.34-3.29 (m, 1H), 2.97-2.87 (m, 1H), 2.78-2.68 (m, 1H).

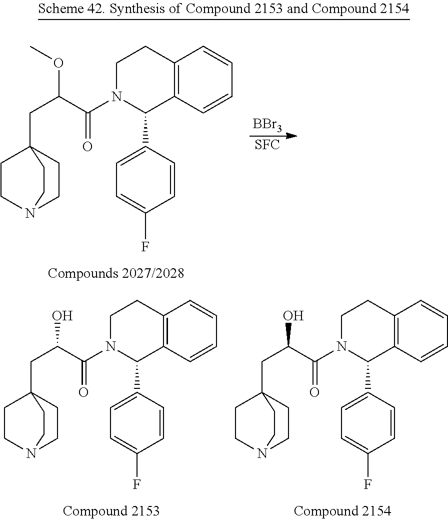

Compound 2153 and Compound 2154 were prepared from Compounds 2027 and Compound 2028, respectively.

To a solution of 1-((S)-1-(4-fluorophenyl)-3,4-dihydroisoquinolin-2(1H)-yl)-2-methoxy-3-(quinuclidin-4-yl)propan-1-one (211 mg, 0.5 mmol) in anhydrous DCM (3 mL) cooled to −78° C., BBr₃ (1.5 mL, 1.5 mmol, 1 M in DCM) was slowly added. The cooling bath was removed and mixture allowed to reach ambient temperature before quenching with saturated aqueous NaHCO₃. This mixture was extracted with three 10 mL portions of DCM and the combined organic phases were washed with water (10 mL), dried over Na₂SO₄, and concentrated. Purification of the residue by prep-HPLC yielded 1-((S)-1-(4-fluorophenyl)-3,4-dihydroisoquinolin-2(1H)-yl)-2-hydroxy-3-(quinuclidin-4-yl)propan-1-one (60 mg) as a white solid.

The diastereomers were separated by chiral SFC eluting with CO₂/MeOH containing 0.2% ammonia/methanol over an EnantioPak® IG column (4.6×100 mm 5 μm) to give Compound 2153 (retention time 2.61 min) and Compound 2154 (retention time 4.18 min). Stereochemical assignment at the 1 position of the tetrahydroisoquinoline is assigned based on the use of chiral starting materials.

Compound 2153: LCMS: (M+H)⁺ 409; purity 94.3% (214 nm); retention time 1.455 min. CP Method A1

¹H NMR (400 MHz, CDCl₃) δ 7.31-7.15 (m, 5H), 7.06 (d, J=7.60 Hz, 1H), 6.97 (t, J=8.4 Hz, 2H), 6.82 (s, 1H), 4.58 (d, J=9.2 Hz, 1H), 3.60-3.55 (m, 1H), 3.45-3.42 (m, 1H), 3.09-3.03 (m, 1H), 2.96-2.90 (m, 6H), 2.22-2.18 (m, 2H), 1.58-1.50 (m, 6H), 1.45-1.40 (m, 2H).

Chiral SFC: MeOH (0.2% ammonia/methanol) over an ENANTIOPAK® IG column (4.6×100 mm 5 μm), retention time 2.61 min).

Compound 2154: LCMS: (M+H)⁺ 409; purity 96.57% (214 nm); retention time 1.453 min. CP Method A1

¹H NMR (400 MHz, CDCl₃) δ 7.30-7.22 (m, 4H), 7.16-7.12 (m, 1H), 7.05 (d, J=7.6 Hz, 1H), 6.99 (t, J=8.4 Hz, 2H), 6.89 (s, 1H), 4.54 (d, J=10.0 Hz, 1H), 3.54-3.50 (m, 1H), 3.39-3.33 (m, 1H), 3.15-3.02 (m, 7H), 2.91-2.85 (m, 2H), 1.77-1.68 (m, 6H), 1.49-1.42 (m, 1H), 1.30-1.25 (m, 1H).

Chiral SFC: MeOH (containing 0.2% methanol ammonia) over an ENANTIOPAK® IG column (4.6×100 mm 5 μm), retention time 4.18 min).

The following compounds were prepared using General Procedure GP-4:

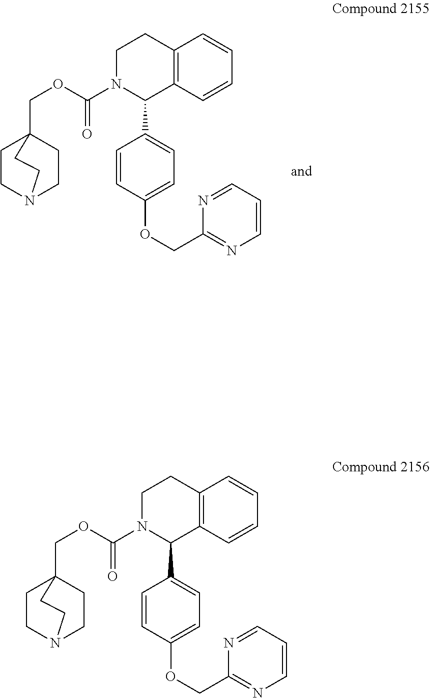 [SMILES: O=C(OCC12CCN(CC1)CC2)N1CCc2ccccc2[C@@H]1c1ccc(OCc2ncccn2)cc1 ; O=C(OCC12CCN(CC1)CC2)N1CCc2ccccc2[C@H]1c1ccc(OCc2ncccn2)cc1]

The diastereomers were separated by chiral SFC eluting with CO₂/EtOH containing 0.5% ammonia/methanol over an EnantioPak® AD column (20×250 mm 10 μm) to give Compound 2155 (retention time=1.54 min) and Compound 2156 (retention time=4.24 min). Stereochemical assignment at the 1 position of the tetrahydroisoquinoline is assigned based on chromatographic elution order as compared to diastereomers of related analogues of known configuration.

Compound 2155: LCMS: (M+H)⁺ 485; purity 100% (214 nm); retention time 1.610 min. CP Method B

¹H NMR (400 MHz, CDCl₃) δ 8.79 (d, J=4.8 Hz, 2H), 7.27 (t, J=5.2 Hz, 1H), 7.24-7.16 (m, 3H), 7.12 (d, J=7.6 Hz, 2H), 7.07 (d, J=7.6 Hz, 1H), 6.93 (d, J=8.8 Hz, 2H), 6.48-6.16 (br, 1H), 5.29 (s, 2H), 4.17-3.92 (br, 1H), 3.85 (dd, J=25.2, 8.4 Hz, 2H), 3.26 (ddd, J=15.2, 10.8, 4.4 Hz, 1H), 3.05-2.87 (m, 7H), 2.78 (dt, J=16.0, 3.6 Hz, 1H), 1.49-1.40 (m, 6H).

Chiral SFC: n-Hexane (0.1% DEA):EtOH (0.1% DEA)=10:90 over an ENANTIOPAK® IG column (4.6×250 mm 5 μm), retention time 30.93 min).

Compound 2156: LCMS: (M+H)⁺ 485; purity 99% (214 nm); retention time 1.597 min. CP Method B

¹H NMR (400 MHz, CDCl₃) δ 8.79 (d, J=4.8 Hz, 2H), 7.29-7.25 (m, 1H), 7.20-7.16 (m, 3H), 7.11 (d, J=8.0 Hz, 2H), 7.07 (d, J=6.8 Hz, 1H), 6.93 (d, J=8.8 Hz, 2H), 6.45-6.10 (br, 1H), 5.29 (s, 2H), 4.10-3.90 (m, 3H), 3.40-3.12 (m, 7H), 2.97 (s, 1H), 2.83 (dt, J=16.0, 4.4 Hz, 1H), 1.70 (s, 6H).

Chiral SFC: n-Hexane (0.1% DEA):EtOH (0.1% DEA)=10:90 over an ENANTIOPAK® IG column (4.6×250 mm 5 μm), retention time 32.98 min).

The following compound was prepared using General Procedure GP-4:

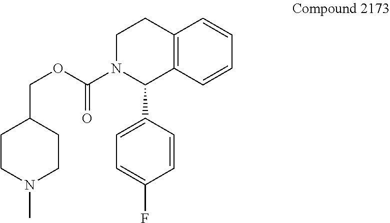 [SMILES: CN1CCC(COC(=O)N2CCc3ccccc3[C@@H]2c2ccc(F)cc2)CC1]

Compound 2173: LCMS: (M+H)⁺ 383; purity 94.9% (214 nm); Retention time 1.71 min. CP Method C

¹H NMR (400 MHz, CDCl₃) δ 7.22 (dd, J=12.5, 5.8 Hz, 5H), 7.03 (d, J=7.3 Hz, 1H), 6.95 (t, J=8.6 Hz, 2H), 6.83 (s, 1H), 4.23 (t, J=8.4 Hz, 2H), 3.94 (dd, J=13.1, 4.5 Hz, 1H), 3.38 (dd, J=17.0, 8.4 Hz, 2H), 3.08 (d, J=4.9 Hz, 1H), 2.83 (d, J=16.2 Hz, 1H), 2.66 (s, 2H), 2.25 (s, 3H), 2.09 (s, 2H), 1.97-1.82 (m, 3H), 1.67-1.58 (m, 2H).

Chiral SFC: 45% MeOH (0.2% ammonia/methanol) over an ENANTIOPAK® IG column (4.6×100 mm 5 μm), retention time 2.096 min).

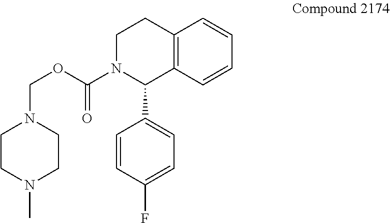 [SMILES: CN1CCN(COC(=O)N2CCc3ccccc3[C@@H]2c2ccc(F)cc2)CC1]

Compound 2174: LCMS: (M+H)⁺ 382.1; purity 100% (214 nm); retention time 1.386 min. CP Method C

¹H NMR (400 MHz, CDCl₃) δ 7.28-7.16 (m, 5H), 7.08 (d, J=7.2 Hz, 1H), 6.98-6.91 (m, 3H), 3.78-3.77 (m, 1H), 3.45-3.29 (m, 1H), 3.01-2.98 (m, 1H), 2.88-2.79 (m, 3H), 2.70-2.47 (m, 11H), 2.31 (s, 3H).

The following compounds were prepared using General Procedure GP-4:

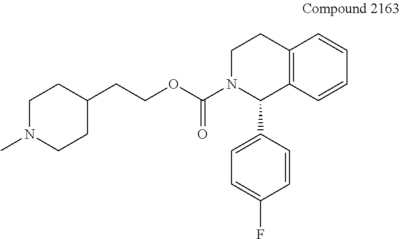 [SMILES: CN1CCC(CCOC(=O)N2CCc3ccccc3[C@@H]2c2ccc(F)cc2)CC1]

Compound 2163: LCMS: (M+H)⁺ 397; purity 100% (214 nm); retention time 1.774 min. CP Method B

¹H NMR (400 MHz, CDCl₃) δ 7.24-7.14 (m, 5H), 7.03 (d, J=7.6 Hz, 1H), 6.97 (t, J=8.8 Hz, 2H), 6.48-6.12 (br, 1H), 4.35-4.40 (m, 3H), 3.36 (d, J=11.6 Hz, 2H), 3.26 (d, J=9.6 Hz, 1H), 2.96 (s, 1H), 2.78 (d, J=16.0 Hz, 1H), 2.63 (s, 3H), 2.45 (s, 2H), 1.90-1.80 (m, 2H), 1.78-1.64 (m, 4H), 1.48 (s, 1H).

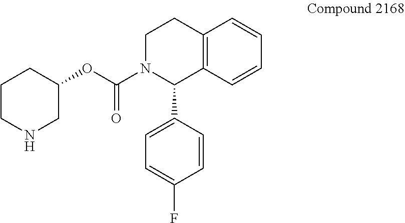 [SMILES: O=C(O[C@H]1CCCNC1)N1CCc2ccccc2[C@@H]1c1ccc(F)cc1]

Compound 2168: LCMS: (M+H)⁺ 355; purity 96% (214 nm); retention time 1.751 min. CP Method B

¹H NMR (400 MHz, CD₃OD) δ7.30-7.18 (m, 6H), 7.11-7.00 (m, 2H), 6.39 (s, 1H), 4.07 (s, 1H), 3.42-3.33 (m, 1H), 3.18 (dd, J=12.8, 2.4 Hz, 1H), 3.05-2.90 (m, 4H), 2.89-2.80 (m, 2H), 2.12-1.92 (m, 1H), 1.91-1.72 (br, 2H), 1.71-1.62 (m, 1H).

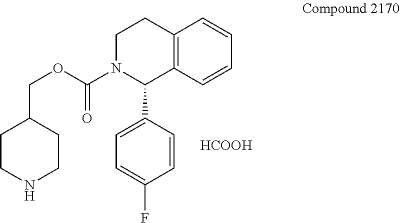 [SMILES: O=C(OCC1CCNCC1)N1CCc2ccccc2[C@@H]1c1ccc(F)cc1 ; "HCOOH"]

Compound 2170: LCMS: (M+H)⁺ 369.2; purity 100% (214 nm); retention time 1.427 min. CP Method C

¹H NMR (400 MHz, CDCl₃) δ 7.25-7.18 (m, 5H), 7.06 (d, J=7.6 Hz, 1H), 7.00-6.96 (m, 2H), 6.41-6.20 (m, 1H), 4.13-4.05 (m, 3H), 3.45 (d, J=11.6 Hz, 2H), 3.31-3.26 (m, 1H), 3.00-2.98 (m, 1H), 2.87-2.78 (m, 3H), 1.96-1.88 (m, 3H), 1.76-1.74 (m, 2H).

The following compounds were prepared using General Procedure GP-1 for urea synthesis:

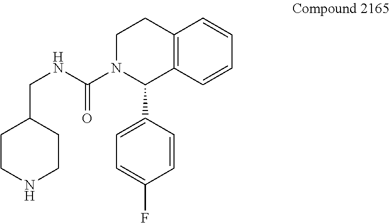 [SMILES: O=C(NCC1CCNCC1)N1CCc2ccccc2[C@@H]1c1ccc(F)cc1]

Compound 2165: LCMS: (M+H)⁺ 368; purity 99% (214 nm); retention time 1.637 min. CP Method B

¹H NMR (400 MHz, CDCl₃) δ 7.25-7.14 (m, 6H), 6.95 (t, J=8.8 Hz, 2H), 6.37 (s, 1H), 4.60 (t, J=5.6 Hz, 1H), 3.56 (t, J=6.4 Hz, 2H), 3.26-3.17 (m, 1H), 3.14-3.06 (m, 3H), 2.90 (dt, J=15.6, 6.4 Hz, 1H), 2.79 (dt, J=16.0, 6.4 Hz, 1H), 2.59 (t, J=12.4 Hz, 2H), 1.71-1.58 (m, 3H), 1.21-1.19 (m, 2H).

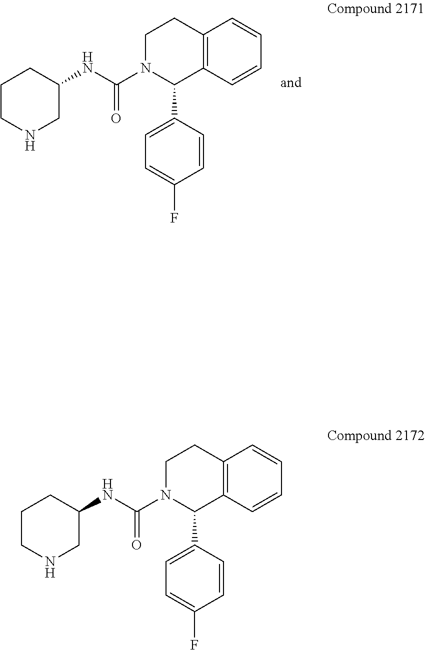 [SMILES: O=C(N[C@H]1CCCNC1)N1CCc2ccccc2[C@@H]1c1ccc(F)cc1 ; O=C(N[C@@H]1CCCNC1)N1CCc2ccccc2[C@@H]1c1ccc(F)cc1]

The diastereomers were separated by chiral SFC eluting with MeOH (0.2% ammonia in methanol) over an EnantioPak® IG (4.6×100 mm 5 □m) to give Compound 2171 (retention time 1.62 min) and Compound 2172 (retention time 2.4 min). Stereochemical assignment at the 1 position of the THIQ motif is based on the use of chiral starting material 5 and the stereochemistry of the piperidine 3-position is based on chromatographic elution order as compared to diastereomers of related analogues of known configuration.

Compound 2171: LCMS: (M+H)⁺ 354.3; purity 100% (214 nm); retention time 1.338 min. CP Method C

¹H NMR (400 MHz, CDCl₃) δ 7.26-7.15 (m, 6H), 6.99-6.95 (m, 2H), 6.42 (s, 1H), 5.34 (s, 1H), 3.98 (s, 1H), 3.73-3.69 (m, 1H), 3.57-3.54 (m, 1H), 3.05-3.01 (m, 1H), 2.95-2.93 (m, 1H), 2.85-2.74 (m, 4H), 1.67-1.59 (m, 3H), 1.51-1.47 (m, 1H).

Compound 2172: LCMS: (M+H)⁺ 354.3; purity 100% (214 nm); retention time 1.338 min. CP Method C

¹H NMR (400 MHz, CDCl₃) δ 7.26-7.16 (m, 6H), 6.98-6.94 (m, 2H), 6.44 (s, 1H), 5.09 (s, 1H), 3.95 (s, 1H), 3.64-3.63 (m, 1H), 3.55-3.53 (m, 1H), 3.04-3.01 (m, 1H), 2.92-2.91 (m, 1H), 2.82-2.78 (m, 3H), 2.59-2.57 (m, 1H), 1.84-1.72 (m, 4H).

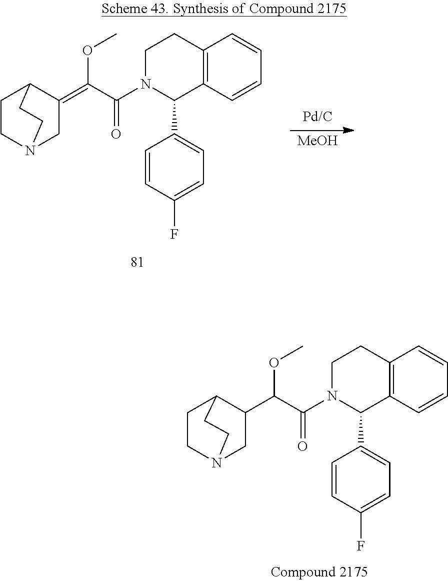 [SMILES: "MeOH" ; CO/C(C(=O)N1CCc2ccccc2[C@@H]1c1ccc(F)cc1)=C1/CN2CCC1CC2 ; COC(C(=O)N1CCc2ccccc2[C@@H]1c1ccc(F)cc1)C1CN2CCC1CC2]

To a solution of (S, E)-1-(1-(4-fluorophenyl)-3, 4-dihydroisoquinolin-2(1H)-yl)-2-hydroxy-2-(quinuclidin-3-ylidene) ethanone 81 (203 mg, 0.5 mmol) in MeOH (5 mL) was added Pd/C (19 mg, 10% w/w). The mixture was placed under a hydrogen atmosphere and stirred at room temperature for 16 h. After removal of the catalyst by filtration, the filtrate was concentrated to obtain an oil. Purification of this oil by prep-HPLC afforded 1-((S)-1-(4-fluorophenyl)-3,4-dihydroisoquinolin-2(1H)-yl)-2-methoxy-2-(quinuclidin-3-yl) ethenone, compound 2175 as a mixture of diastereomers.

Compound 2175: LCMS: (M+H)⁺ 409, purity 100% (214 nm), Retention time 1.629 min. CP Method C

¹H NMR (400 MHz, CDCl₃) δ 8.58 (s, 1H), 7.24-7.09 (m, 5H), 7.07-6.88 (m, 3H), 6.79 (dd, J=32.1, 16.4 Hz, 1H), 4.78 (dd, J=75.6, 6.2 Hz, 1H), 4.44 (dd, J=15.6, 7.5 Hz, 1H), 4.25 (dd, J=21.1, 12.8 Hz, 1H), 3.95-3.74 (m, 1H), 3.51 (dd, J=18.6, 11.9 Hz, 2H), 3.41-3.19 (m, 3H), 3.10-2.95 (m, 3H), 2.93-2.80 (m, 1H), 2.74-2.60 (m, 1H), 2.52-2.37 (m, 1H), 2.33-2.17 (m, 1H), 2.11-1.85 (m, 2H), 1.76 (s, 1H), 1.65-1.48 (m, 1H), 1.30 (dd, J=19.2, 11.8 Hz, 1H).

The following compounds were prepared using General Procedure GP-3:

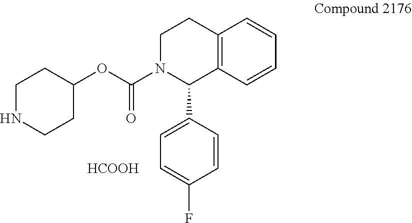 [SMILES: O=C(OC1CCNCC1)N1CCc2ccccc2[C@@H]1c1ccc(F)cc1 ; "HCOOH"]

Compound 2176: LCMS: (M+H)⁺ 355; purity 98.9% (214 nm); Retention time 1.46 min. CP Method A1

¹H NMR (400 MHz, CDCl₃) δ 8.52 (s, 1H), 7.20 (dt, J=13.3, 6.2 Hz, 5H), 7.04 (d, J=7.3 Hz, 1H), 6.98 (d, J=7.4 Hz, 2H), 6.40 (s, 1H), 5.01 (s, 1H), 3.98 (s, 1H), 3.24 (s, 3H), 3.08 (s, 2H), 2.97 (s, 1H), 2.81 (d, J=16.0 Hz, 1H), 2.15 (s, 2H), 1.98 (s, 2H).

Chiral SFC: 35% MeOH (0.2% ammonia/methanol) over an ENANTIOPAK® IG column (4.6×100 mm 5 μm), retention time 1.484 min).

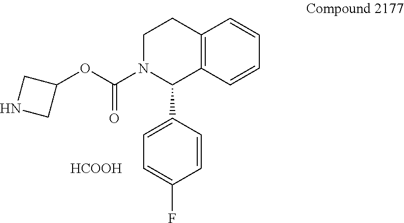 [SMILES: O=C(OC1CNC1)N1CCc2ccccc2[C@@H]1c1ccc(F)cc1 ; "HCOOH"]

Compound 2177: LCMS: (M+H)⁺ 327; purity 100% (214 nm); Retention time 1.37 min. CP Method A1

¹H NMR (400 MHz, CDCl₃) δ 8.51 (s, 1H), 7.25-7.13 (m, 5H), 7.00 (dd, J=16.2, 7.3 Hz, 3H), 6.30 (d, J=30.3 Hz, 1H), 5.33 (s, 1H), 4.25 (s, 2H), 4.02 (s, 3H), 3.26 (d, J=10.0 Hz, 1H), 3.01 (ddd, J=16.4, 10.8, 5.8 Hz, 1H), 2.80 (d, J=16.3 Hz, 1H).

Chiral SFC: 20% MeOH (0.2% ammonia/methanol) over an ENANTIOPAK® IG column (4.6×100 mm 5 μm), retention time 3.906 min).

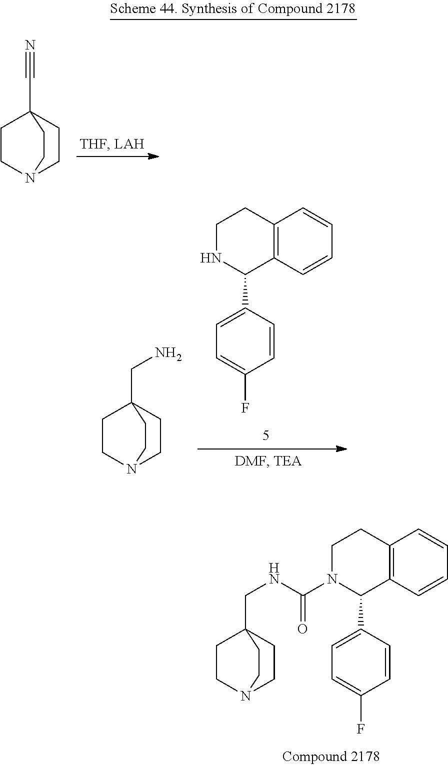 [SMILES: N#CC12CCN(CC1)CC2 ; NCC12CCN(CC1)CC2 ; Fc1ccc([C@@H]2NCCc3ccccc32)cc1 ; O=C(NCC12CCN(CC1)CC2)N1CCc2ccccc2[C@@H]1c1ccc(F)cc1]

Step 1: To a solution of quinuclidine-4-carbonitrile 162 (1.36 g, 10 mmol) in THF (35 mL) was added LiAlH₄ (1 M in THF, 20 mL, 20 mmol). The mixture was stirred at room temperature for 2 hours, then the reaction was quenched with water. The mixture was filtered and the filter cake was washed with THF (50 mL×3). The filtrate was dried over Na₂SO₄, filtered, and concentrated to give crude quinuclidin-4-ylmethanamine 163 (500 mg) as a white solid which used for the next step without further purification.

Step 2: Compound 2178 was prepared following the General Procedure GP-1 for urea synthesis.

Compound 2178: LCMS: (M+H)⁺=394; retention time=1.396 min. Method C

¹H NMR (400 MHz, DMSO-d₆) δ 7.27-7.07 (m, 8H), 6.42 (d, J=8.7 Hz, 1H), 6.36 (t, J=6.1 Hz, 1H), 3.82-3.70 (m, 1H), 3.47 (s, 1H), 2.96 (dd, J=13.5, 6.4 Hz, 1H), 2.84 (ddd, J=19.1, 11.1, 5.6 Hz, 2H), 2.70-2.59 (m, 6H), 1.74 (s, 1H), 1.34-1.04 (m, 6H). Scheme 45. Synthesis of Compound 2179

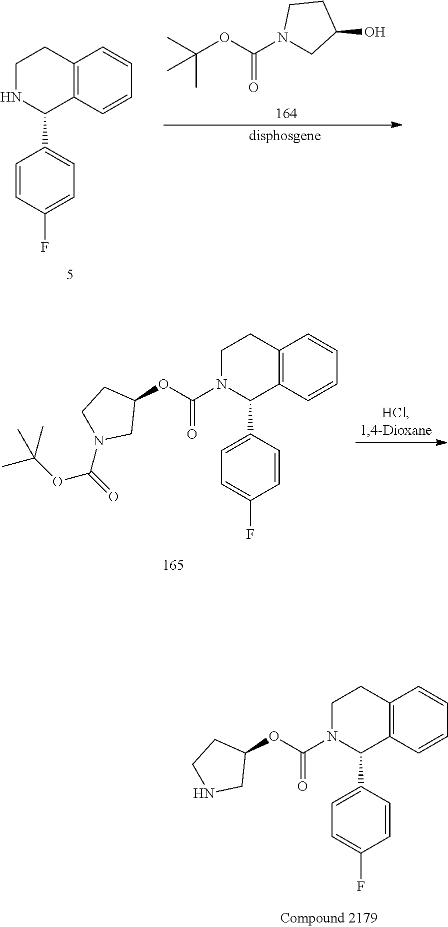

Step 1: To a solution of (R)-tert-butyl 3-hydroxypyrrolidine-1-carboxylate 164 (1870 mg, 10 mmol) in MeCN (100 mL) was added diphosgene (1970 mg, 10 mmol) and the mixture was stirred at room temperature for 2 hours. The mixture was concentrated to give a white solid. The white solid was dissolved in 25 mL of DMF and (S)-1-(4-fluorophenyl)-1,2,3,4-tetrahydroisoquinoline 5 (1140 mg, 5 mmol) and TEA (2000 mg, 20 mmol) were added. The mixture was stirred at 60° C. for 2.5 hours. The mixture was cooled to 0° C. and water (50 mL) was added. The mixture was extracted with two 50 mL portions of ethyl acetate. The combine organic layers were washed with 50 mL brine, dried and concentrated in vacuo to give crude product. The crude product was purified by column chromatography eluting with petroleum ether/ethyl acetate (3:1) to give 100 mg of (1S)—((R)-1-(tert-butoxycarbonyl)pyrrolidin-3-yl)-1-(4-fluorophenyl)-3,4-dihydroisoquinoline-2(1H)-carboxylate as a white solid.

LCMS: (M+H)⁺=441 (UV 214 nm); Retention time=1.695 min. Method C

Step 2: A solution of (1 S)—((R)-1-(tert-butoxycarbonyl)pyrrolidin-3-yl)-1-(4-fluorophenyl)-3,4-dihydroisoquinoline-2(1H)-carboxylate (100 mg, 0.23 mol) in HCl in 1,4-dioxane (10 mL) was stirred at room temperature for 2 hours. The mixture was concentrated in vacuo to give crude product which was purified by prep-HPLC to give (1S)—((R)-pyrrolidin-3-yl)-1-(4-fluorophenyl)-3,4-dihydroisoquinoline-2(1H)-carboxylate, compound 2179.

Compound 2179: LCMS: (M+H)⁺=341; retention time=1.688 min. Method C

¹H NMR (400 MHz, CDCl₃) δ 7.25-7.11 (m, 5H), 7.10-6.84 (m, 3H), 6.30 (d, J=87.3 Hz, 1H), 5.25 (dd, J=6.3, 4.6 Hz, 1H), 4.06 (d, J=58.1 Hz, 1H), 3.29-3.16 (m, 1H), 3.15-2.85 (m, 5H), 2.77 (d, J=15.2 Hz, 1H), 2.05 (td, J=14.4, 7.0 Hz, 1H), 1.87 (s, 1H).

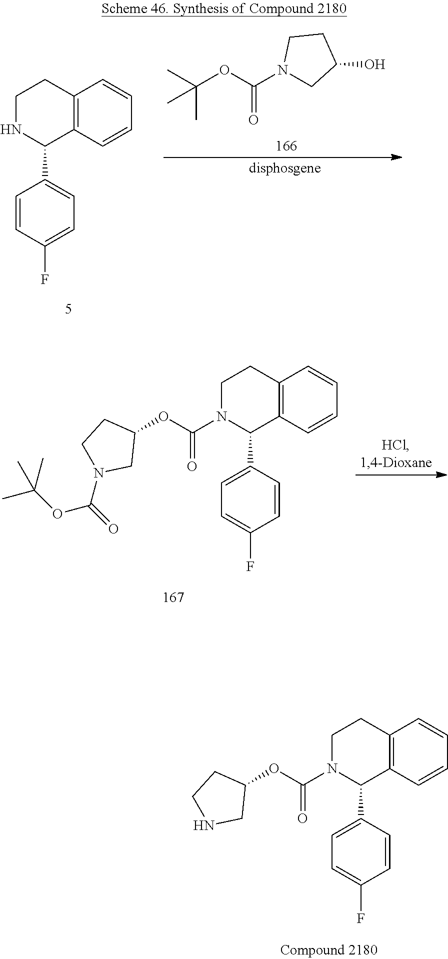

Step 1: To a solution of (S)-tert-butyl 3-hydroxypyrrolidine-1-carboxylate 166 (1870 mg, 10 mmol) in MeCN (100 mL) was added diphosgene (1970 mg, 10 mmol) and the mixture was stirred at room temperature for 2 hours. The mixture was concentrated to give a white solid. The white solid was dissolved in 25 mL of DMF and (S)-1-(4-fluorophenyl)-1,2,3,4-tetrahydroisoquinoline 5 (1140 mg, 5 mmol) and TEA (2000 mg, 20 mmol) was added. The mixture was stirred at 60° C. for 2.5 hours. The mixture was cooled to 0° C. and water (50 mL) was added. The mixture was extracted with two 50 mL portions of ethyl acetate. The combined organic layers were washed with 50 mL brine, dried and concentrated in vacuo to give crude product. The crude product was purified by column chromatography eluting with petroleum ether/ethyl acetate (3:1) to give 100 mg of (1S)—((S)-1-(tert-butoxycarbonyl)pyrrolidin-3-yl)-1-(4-fluorophenyl)-3,4-dihydroisoquinoline-2(1H)-carboxylate 167 as a white solid.

LCMS: (M+H)⁺=441 (UV 214 nm); Retention time=1.708 min. Method C

Step 2: A solution of (1S)—((S)-1-(tert-butoxycarbonyl)pyrrolidin-3-yl)-1-(4-fluorophenyl)-3,4-dihydroisoquinoline-2(1H)-carboxylate 167 (100 mg, 0.23 mol) in HCl-1,4-dioxane 10 mL was stirred at room temperature for 2 h. The mixture was concentrated in vacuo to give the crude product which was purified by prep-HPLC to give (1S)—((S)-pyrrolidin-3-yl)-1-(4-fluorophenyl)-3,4-dihydroisoquinoline-2(1H)-carboxylate.

Compound 2180: LCMS: (M+H)⁺=341; retention time=1.506 min. Method C

¹H NMR (400 MHz, CDCl₃) δ 7.25-7.11 (m, 5H), 7.10-6.84 (m, 3H), 6.30 (d, J=87.3 Hz, 1H), 5.25 (dd, J=6.3, 4.6 Hz, 1H), 4.06 (d, J=58.1 Hz, 1H), 3.29-3.16 (m, 1H), 3.15-2.85 (m, 5H), 2.77 (d, J=15.2 Hz, 1H), 2.05 (td, J=14.4, 7.0 Hz, 1H), 1.87 (s, 1H).

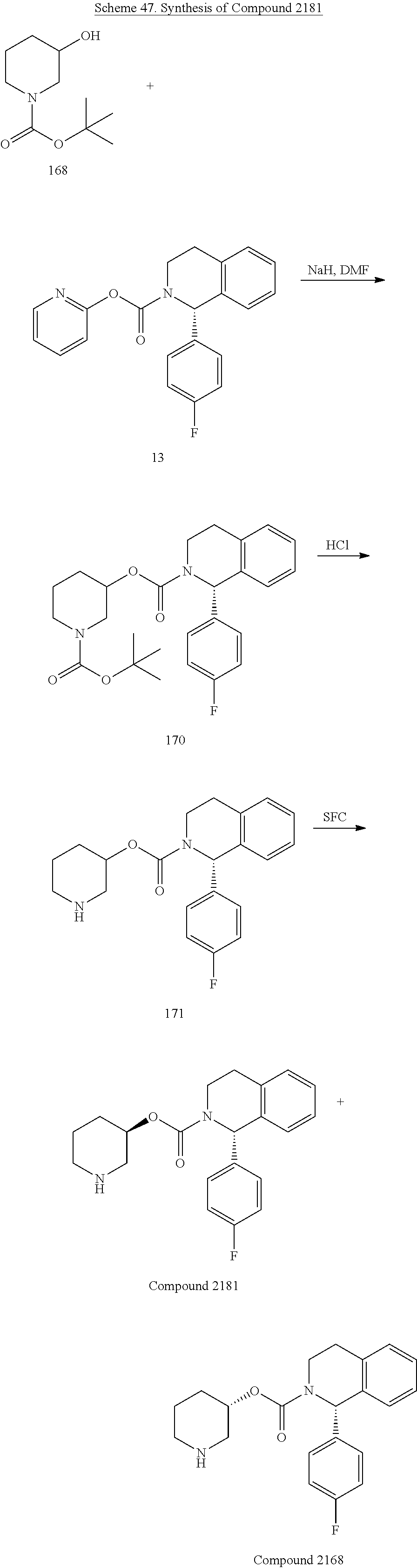

Step 1: To a solution of tert-butyl 3-hydroxypiperidine-1-carboxylate 13 (0.1 g, 0.5 mmol) in dry DMF (1 mL) was added NaH (60 mg, 1.5 mmol) at 0° C. The reaction was stirred at room temperature for 30 min, then a solution of (S)-pyridin-2-yl 1-(4-fluorophenyl)-3,4-dihydroisoquinoline-2(1H)-carboxylate 5 (173 mg, 0.5 mmol) in dry DMF (1 mL) was added to the reaction at 0° C. The reaction was heated to 65° C. for 2 hrs. LCMS showed the starting material was consumed. The reaction was poured into water (15 mL) and extracted with three 20 mL portions of DCM. The combined organic layers were dried and concentrated to give crude (1 S)-1-(tert-butoxycarbonyl)piperidin-3-yl 1-(4-fluorophenyl)-3,4-dihydroisoquinoline-2(1H)-carboxylate 170 (0.22 g, 0.5 mmol) (theory mass) as a light brown oil used in the next step without further purification.

LCMS: (M−55)⁺=399; Retention time=2.41 min. Method A1

Step 2: To a solution of crude (1S)-1-(tert-butoxycarbonyl)piperidin-3-yl 1-(4-fluorophenyl)-3,4-dihydroisoquinoline-2(1H)-carboxylate 3 (0.22 g, 0.5 mmol) 170 in DCM (2 mL) was added 4M HCl/dioxane (0.5 mL, 2 mmol) at room temperature. The reaction mixture was stirred at room temperature for 3 hours. The reaction mixture was concentrated and the residue was purified by prep-HPLC to give (1S)-piperidin-3-yl 1-(4-fluorophenyl)-3,4-dihydroisoquinoline-2(1H)-carboxylate 171 (50 mg, 0.14 mmol) as a white solid.

LCMS: (M+1)⁺=355; Retention time=1.75 min. Method A1

The diastereomers were separated by chiral SFC eluting with CO₂/MeOH containing 0.2% methanolic ammonia over an EnantioPak® IG column (4.6×100 mm 5 μm) to give Compound 2181 (retention time=3.33 min) and Compound 2168 (retention time=1.92 min). Stereochemical assignment of (S) at the 1 position of the tetrahydroisoquinoline is assigned based on starting materials of known of known configuration. Stereochemical assignment at the piperidine juncture is assigned based on elution order from chiral SFC.

Compound 2181: LCMS: (M+1)⁺=355; Retention time=1.80 min. Method C

HNMR (400 Hz, DMSO-d₆): δ 7.24-7.12 (m, 8H), 6.26 (br, 1H), 4.55-4.49 (m, 1H), 3.89 (br, 1H), 3.32-3.25 (m, 1H), 2.92-2.81 (m, 3H), 2.68-2.45 (m, 2H), 1.82 (br, 1H), 1.86-1.35 (m, 3H).

Compound 2168: LCMS: (M+1)⁺=355; Retention time=1.81 min. Method C

HNMR (400 Hz, DMSO-d₆): δ 7.25-7.12 (m, 8H), 6.31 (br, 1H), 4.73 (s, 1H), 3.93 (t, J=6.4 Hz, 1H), 3.26-2.80 (m, 7H), 1.88-1.52 (m, 4H).

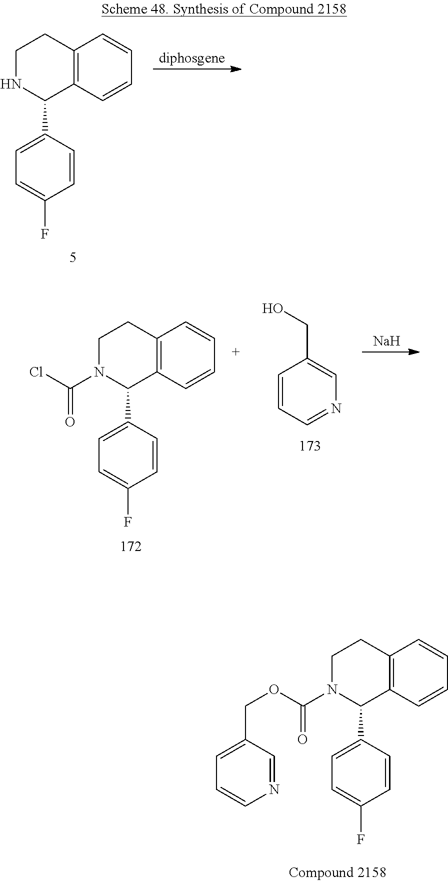

Step 1: To a solution of (S)-1-(4-fluorophenyl)-1,2,3,4-tetrahydroisoquinoline 5 (227 mg, 1 mmol) in MeCN (5 mL) was added diphosgene (0.18 mL, 1.5 mmol) and the mixture stirred at room temperature for 1 hr. Then the solution was concentrated under reduced pressure to give (S)-1-(4-fluorophenyl)-3,4-dihydroisoquinoline-2(1H)-carbonyl chloride 172 as a white solid (289 mg) which was used for the next step without any purification.

Step 2: To a solution of pyridin-3-ylmethanol 173 (0.15 mL, 1.5 mmol) in DMF (5 mL) was added NaH (72 mg, 3 mmol) at room temperature and the mixture was stirred for 20 minutes. (S)-1-(4-Fluorophenyl)-3,4-dihydroisoquinoline-2(1H)-carbonyl chloride 172 (289 mg, 1 mmol) was added and the mixture was heated to 80° C. and stirred overnight. After cooling to room temperature, 5 mL water was added to quench the reaction. The mixture was extracted with three 10 mL portions of DCM. The combined organic phase was washed with 10 mL brine, 10 mL water, dried and concentrated to give a residue. The residue was purified by prep-HPLC to give (S)-pyridin-3-ylmethyl 1-(4-fluorophenyl)-3,4-dihydroisoquinoline-2(1H)-carboxylate, Compound 2158.

Compound 2158: LCMS: (M+H)⁺=363.1; purity=100% (214 nm); retention time=1.431 min. Method C

¹H NMR (400 MHz, CDCl₃) δ 8.65 (d, J=24.4 Hz, 2H), 7.77 (s, 1H), 7.38-7.35 (m, 1H), 7.27-7.18 (m, 4H), 7.04-6.96 (m, 4H), 6.45-6.31 (m, 1H), 5.27-5.22 (m, 1H), 4.15-4.03 (m, 1H), 3.29-3.28 (m, 1H), 3.01 (s, 1H), 2.81 (d, J=15.2 Hz, 1H), 2.57 (s, 1H).

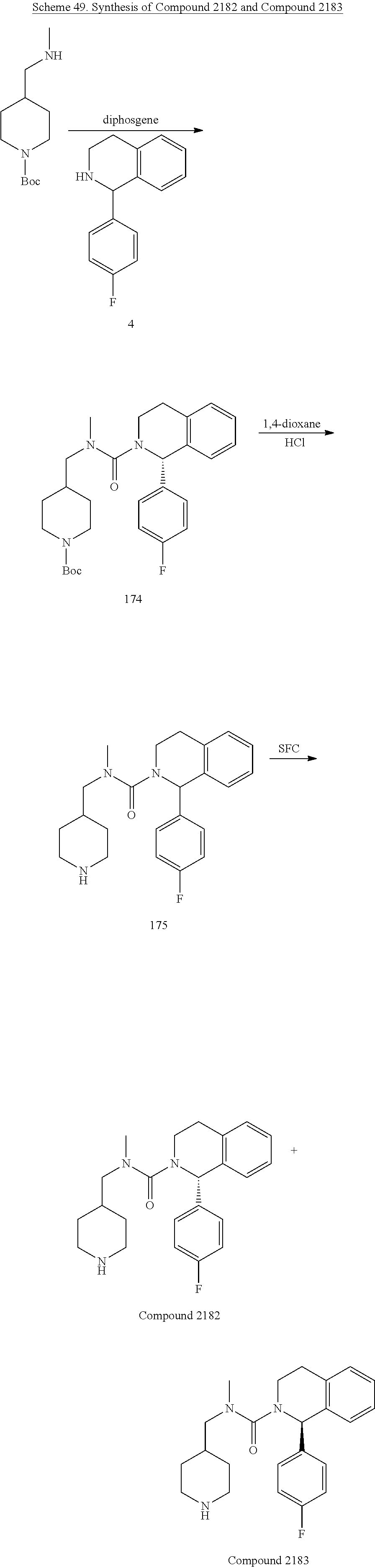

Step 1: To a solution of tert-butyl 4-((methylamino)methyl)piperidine-1-carboxylate (228 mg, 1 mmol) in MeCN (10 mL) was added triphosgene (297 mg, 1.5 mmol). The mixture was stirred at room temperature for 2 hours and then concentrated to give a white solid. The solid was dissolved in DMF (5 mL) and 1-(4-fluorophenyl)-1,2,3,4-tetrahydroisoquinoline 4 (228 mg, 1 mmol) and TEA (202 mg, 2 mmol) were added. The mixture was stirred at 60° C. for 2 hours before cooling to ambient temperature. Water (10 mL) was added and the mixture was extracted with three 20 mL portions of ethyl acetate. The organic phase was washed with three 20 mL portions of brine, dried and concentrated in vacuo. The crude product was purified by flash column chromatography eluting with PE/EA (3:1) to afford tert-butyl 4-((1-(4-fluorophenyl)-N-methyl-1,2,3,4-tetrahydroisoquinoline-2-carboxamido)methyl)piperidine-1-carboxylate, 174 (120 mg) as a yellow oil.

LCMS: (M−57)⁺ 426; purity 41% (214 nm); retention time 2.267 min. by CP Method E

Step 2: To a solution of tert-butyl 4-((1-(4-fluorophenyl)-N-methyl-1,2,3,4-tetrahydroisoquinoline-2-carboxamido)methyl)piperidine-1-carboxylate 174 (120 mg, 0.25 mmol) in DCM (10 mL) was added HCl (2 mL in 1.4-dioxane). The mixture was stirred at room temperature for 2 hours and then diluted with saturated NaHCO₃ (20 mL). After extraction with three 20 mL portions of dichloromethane, the combined organic phase was dried and concentrated in vacuo. The crude product was purified by HPLC (Mobile Phase: A: H₂O (10 mM NH₄HCO₃) B: MeCN Gradient: 5%-95% B in 1.2 min, Flow Rate: 2.0 mL/min Column: XBridge C18 50×4.6 mm, 3.5 μm, Oven Temperature: 40° C. UV214, MASS:100-1000) to afford 1-(4-fluorophenyl)-N-methyl-N-(piperidin-4-ylmethyl)-3,4-dihydroisoquinoline-2(1H)-carboxamide, 175 (50 mg) as a brown solid.

The diastereomers were separated by chiral SFC eluting with CO₂/MeOH containing 0.2% methanolic ammonia over an EnantioPak® AD column (4.6×100 mm, 5 μm) to give Compound 2182 (retention time 2.14 min) and Compound 2183 (retention time 3.76 min). Stereochemical assignment at the 1 position of the tetrahydroisoquinoline is assigned based on chromatographic elution order as compared to diastereomers of related analogues of known configuration.

Compound 2182: LCMS: (M+H)⁺ 382; purity 98% (214 nm); retention time 1.394 min. by CP Method E

¹H NMR (400 MHz, CDCl₃) δ 7.24-7.13 (m, 5H), 7.01-6.93 (m, 3H), 6.07 (s, 1H), 3.63-3.52 (m, 1H), 3.37-3.27 (m, 2H), 3.19-3.07 (m, 2H), 3.05-2.90 (m, 3H), 2.88 (s, 3H), 2.67-2.54 (m, 2H), 1.81-1.73 (m, 1H), 1.61 (t, J=11.2 Hz, 2H), 1.25-1.03 (m, 2H).

Chiral SFC: MeOH (containing 0.2% ammonia/methanol) over an ENANTIOPAK® IG column (4.6×100 mm, 5 μm), retention time 2.68 min).

Compound 2183: LCMS: (M+H)⁺ 382; purity 100% (214 nm); retention time 1.394 min. by CP Method E

¹H NMR (400 MHz, CDCl₃) δ 7.24-7.13 (m, 5H), 7.01-6.93 (m, 3H), 6.07 (s, 1H), 3.63-3.52 (m, 1H), 3.40-3.26 (m, 4H), 3.05-2.93 (m, 3H), 2.89 (s, 3H), 2.78-2.66 (m, 2H), 1.91-1.82 (m, 1H), 1.73 (t, J=17.2 Hz, 2H), 1.54-1.36 (m, 2H).

Chiral SFC: MeOH (containing 0.2% ammonia/methanol) over an ENANTIOPAK® IG column (4.6×100 mm, 5 μm), retention time 4.11 min).

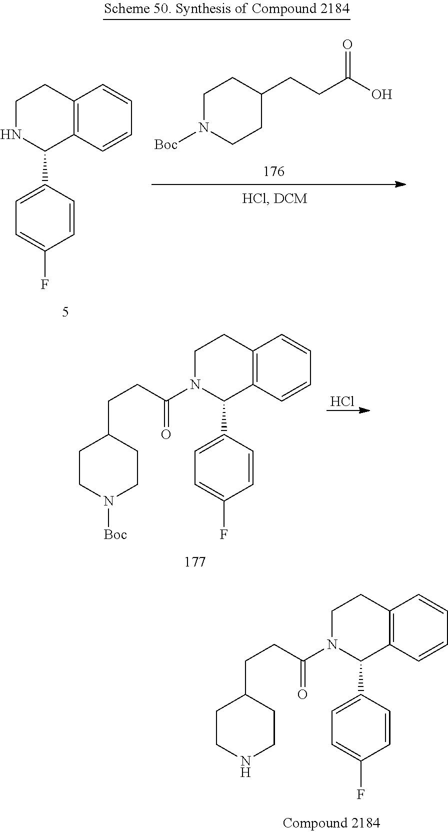

Step 1: (S)-tert-butyl 4-(3-(1-(4-fluorophenyl)-3,4-dihydroisoquinolin-2(1H)-yl)-3-oxopropyl)piperidine-1-carboxylate was prepared following the General Procedure CP-5 for amide synthesis.

Step 2: To a solution of (S)-tert-butyl 4-(3-(1-(4-fluorophenyl)-3,4-dihydroisoquinolin-2(1H)-yl)-3-oxopropyl)piperidine-1-carboxylate (100 mg, 0.21 mmol) in DCM (10 mL) was added HCl (2 mL, in 1.4-dioxane). The mixture was stirred at room temperature for 2 hours, diluted with saturated NaHCO₃ (20 mL) and extracted with three 20 mL portions of dichloromethane. The organic layers phase was dried, concentrated in vacuo and the crude product purified by prep-HPLC to give (S)-1-(1-(4-fluorophenyl)-3,4-dihydroisoquinolin-2(1H)-yl)-3-(piperidin-4-yl)propan-1-one, Compound 2184.

Compound 2184: LCMS: (M+H)⁺ 367.2; purity 98.08% (214 nm); retention time 1.460 min. by CP Method C

¹H NMR (400 MHz, CDCl₃) δ 7.26-7.18 (m, 5H), 7.07 (d, J=7.2 Hz, 1H), 6.98-6.94 (m, 2H), 6.90 (s, 1H), 3.78-3.74 (m, 1H), 3.47-3.35 (m, 3H), 2.99-2.95 (m, 1H), 2.89 (s, 1H), 2.85-2.79 (m, 3H), 2.45-2.40 (m, 2H), 1.88-1.85 (m, 2H), 1.71 (s, 2H), 1.58 (s, 3H).

The following compounds were prepared analogously:

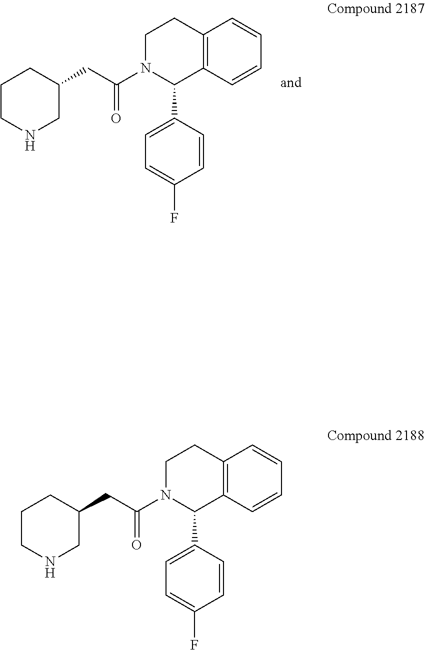 [SMILES: O=C(C[C@H]1CCCNC1)N1CCc2ccccc2[C@@H]1c1ccc(F)cc1 ; O=C(C[C@@H]1CCCNC1)N1CCc2ccccc2[C@@H]1c1ccc(F)cc1]

The diastereomers were separated by chiral SFC eluting with CO₂/MeOH containing 0.2% ammonia/methanol over an EnantioPak® OZ-H column (4.6×100 mm, 5 μm) to give Compound 2187 (retention time 1.57 min) and Compound 2188 (retention time 2.13 min). Stereochemical assignment of (S) at the 1 position of the tetrahydroisoquinoline is assigned based on chiral starting materials. Stereochemical assignment at the piperidine juncture is based chromatographic elution order as compared to diastereomers of related analogues of known configuration.

Compound 2187: LCMS: (M+H)⁺ 353.2; purity 100% (214 nm); retention time 1.387 min. by CP Method C

¹H NMR (400 MHz, CDCl₃) δ 7.26-7.17 (m, 5H), 7.07 (d, J=7.2 Hz, 1H), 6.95-6.93 (m, 3H), 3.82-3.79 (m, 1H), 3.47-3.41 (m, 1H), 3.11-3.08 (m, 1H), 3.03-2.97 (m, 2H), 2.87-2.83 (m, 1H), 2.62-2.52 (m, 1H), 2.41-2.32 (m, 2H), 2.28-2.07 (m, 1H), 1.89-1.86 (m, 1H), 1.78-1.65 (m, 2H), 1.52-1.49 (m, 1H), 1.19-1.16 (m, 1H).

Chiral SFC: MeOH (containing 0.2% ammonia/methanol) over an ENANTIOPAK® IG column (4.6×100 mm, 5 μm), retention time 2.57 min.

Compound 2188: LCMS: (M+H)⁺ 353.2; purity 100% (214 nm); retention time 1.389 min. by CP Method C

¹H NMR (400 MHz, CDCl₃) δ 7.25-7.15 (m, 5H), 7.05 (d, J=7.2 Hz, 2H), 6.98-6.94 (m, 2H), 6.87 (s, 1H), 3.81-3.71 (m, 1H), 3.47-3.43 (m, 2H), 3.31-3.27 (m, 1H), 3.11-3.04 (m, 1H), 2.89-2.84 (m, 2H), 2.58-2.55 (m, 1H), 2.51-2.46 (m, 2H), 1.90 (s, 4H), 1.40-1.37 (m, 1H).

Chiral SFC: MeOH (containing 0.2% ammonia/methanol) over an ENANTIOPAK® IG column (4.6×100 mm, 5 μm), retention time 11.62 min).

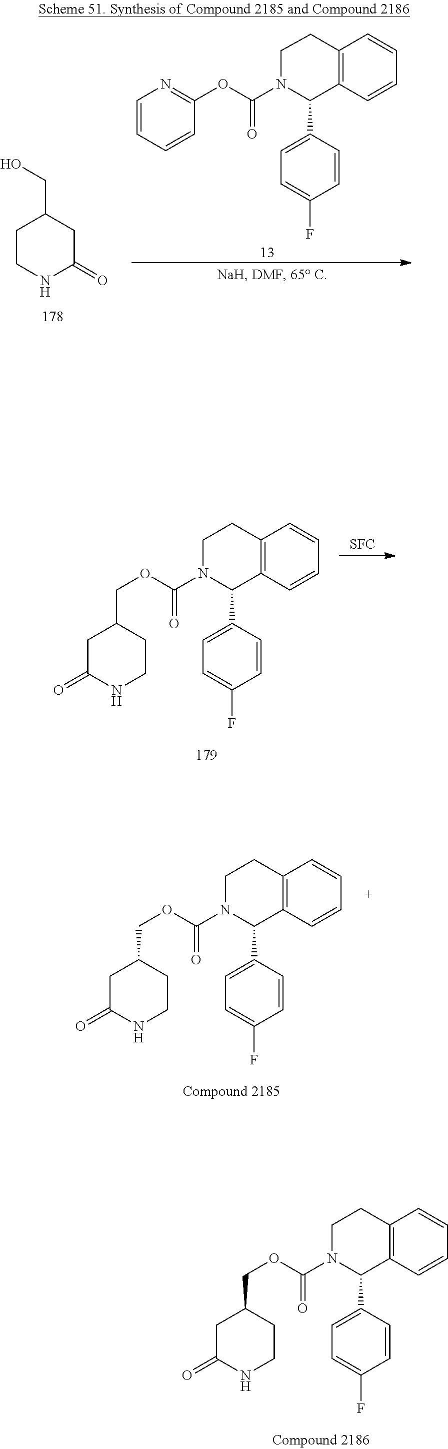

Step 1: To a solution of 4-(hydroxymethyl)piperidin-2-one 178 (80 mg, 0.62 mmol) in dry DMF (2 mL), cooled to 0° C., was added NaH (50 mg, 1.24 mmol, 60% in mineral oil). After the reaction mixture was stirred at room temperature for 30 minutes, it was re-cooled to 0° C. and a solution of (S)-pyridin-2-yl 1-(4-fluorophenyl)-3,4-dihydroisoquinoline-2(1H)-carboxylate 13 (259 mg, 0.74 mmol) in dry DMF (1 mL) was added. The reaction mixture was heated to 65° C. for 2 hours and then poured into water and extracted with three 20 mL portions of DCM. The combined organic phase was dried, concentrated and the crude residue purified by prep-HPLC to afford (1 S)-(2-oxopiperidin-4-yl)methyl 1-(4-fluorophenyl)-3,4-dihydroisoquinoline-2(1H)-carboxylate 179 (58 mg, 0.15 mmol) as a white solid.

LCMS: (M+H)⁺ 383; Retention time 1.89 min. by CP Method A1

Step 2: The diastereomers were separated by chiral SFC eluting with CO₂/MeOH containing 0.2% ammonia/methanol over an EnantioPak® AS-H column (4.6×100 mm, 5 μm) to give Compound 2185 (retention time 3.46 min) and Compound 2186 (retention time 2.64 min). Stereochemical assignment at the 1 position of the tetrahydroisoquinoline is assigned based on chromatographic elution order as compared to diastereomers of related analogues of known configuration.

Compound 2185: LCMS: (M+H)⁺ 383; Retention time 1.71 min. by CP Method C

HNMR (400 Hz, DMSO-d₆): δ 7.49 (s, 1H), 7.25-7.12 (m, 8H), 6.26 (br, 1H), 4.03-3.85 (m, 3H), 3.11 (t, J=11 Hz, 2H), 2.93-2.78 (m, 2H), 2.22-2.11 (m, 2H), 1.96-1.76 (m, 2H), 1.36 (br, 1H).

Chiral SFC: MeOH (containing 0.2% ammonia/methanol) over an ENANTIOPAK® IG column (4.6×100 mm, 5 μm), retention time 3.72 min.

Compound 2186: LCMS: (M+H)⁺ 383; Retention time 1.71 min. by CP Method C

HNMR (400 Hz, DMSO-d₆): δ 7.49 (s, 1H), 7.25-7.12 (m, 8H), 6.26 (br, 1H), 4.02-3.86 (m, 3H), 3.12 (d, J=12 Hz, 2H), 2.93-2.78 (m, 2H), 2.24-2.08 (m, 2H), 1.94-1.70 (m, 2H), 1.36 (br, 1H).

Chiral SFC: MeOH (containing 0.2% ammonia/methanol) over an ENANTIOPAK® IG column (4.6×100 mm, 5 μm), retention time 3.75 min).

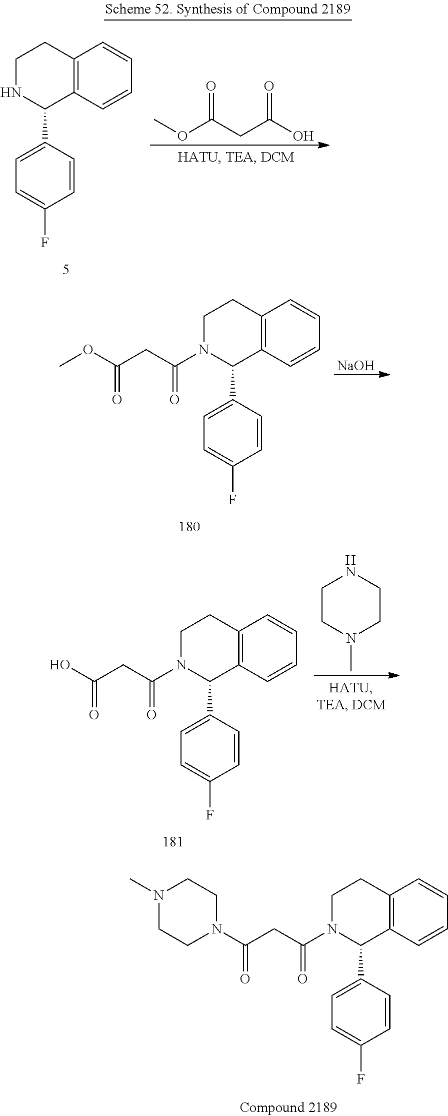

Step 1: A solution of (S)-1-(4-fluorophenyl)-1, 2, 3, 4-tetrahydroisoquinoline 5 (2.27 g, 10 mmol), HATU (5.7 g, 15 mmol), 3-methoxy-3-oxopropanoic acid (1.18 mg, 10 mmol) and TEA (3.03 g, 30 mmol) in DCM (50 mL) was stirred at room temperature for 16 hrs. The mixture was diluted with water (50 mL), and the organic phase washed with brine (50 mL), dried over anhydrous Na₂SO₄, filtered and concentrated. The light-yellow crude oil was purified by prep-HPLC to obtain (S)-methyl 3-(1-(4-fluorophenyl)-3, 4-dihydroisoquinolin-2(1H)-yl)-3-oxopropanoate, 180 (0.65 g).

LCMS: (M+H)⁺ 328; purity 100% (214 nm); Retention time 1.90 min. by CP Method A1

Step 2: To a solution of (S)-methyl 3-(1-(4-fluorophenyl)-3, 4-dihydroisoquinolin-2(1H)-yl)-3-oxopropanoate 180 (650 mg, 2 mmol) in MeOH (5 mL) and water (5 mL) was added NaOH (320 mg, 8 mmol). The mixture was stirred at room temperature for 2 hours and then concentrated. The pH of the residue was adjusted to 5, then it was purified by prep-HPLC to afford (S)-3-(1-(4-fluorophenyl)-3, 4-dihydroisoquinolin-2(1H)-yl)-3-oxopropanoic acid, 181 (260 mg).

LCMS: (M+H)⁺ 314; purity 100% (214 nm); Retention time 1.78 min. by CP Method A1

Step 3: A solution of (S)-3-(1-(4-fluorophenyl)-3, 4-dihydroisoquinolin-2(1H)-yl)-3-oxopropanoic acid 181 (100 mg, 0.32 mmol), HATU (182 mg, 0.48 mmol), 1-methylpiperazine (32 mg, 0.32 mmol) and TEA (98 mg, 0.96 mmol) in DCM (5 mL) was stirred at room temperature for 16 hours. The mixture was diluted with water (5 mL), the organic phase washed with brine (5 mL), dried over anhydrous Na₂SO₄, filtered and concentrated. The crude light-yellow oil was purified by prep-HPLC to obtain (S)-1-(1-(4-fluorophenyl)-3, 4-dihydroisoquinolin-2(1H)-yl)-3-(4-methylpiperazin-1-yl) propane-1, 3-dione, compound 2189 (23 mg).

Compound 2189: LCMS: (M+H)⁺ 396; purity 99.7% (214 nm); Retention time 1.64 min. by CP Method C

¹H NMR (400 MHz, CDCl₃) δ 7.24 (d, J=6.8 Hz, 1H), 7.22-7.16 (m, 4H), 7.07-7.02 (m, 1H), 6.95 (t, J=8.6 Hz, 2H), 6.83 (s, 1H), 4.02 (d, J=11.2 Hz, 1H), 3.79-3.70 (m, 4H), 3.65-3.56 (m, 2H), 3.52-3.42 (m, 1H), 3.02 (dd, J=11.0, 5.8 Hz, 1H), 2.85 (d, J=16.3 Hz, 1H), 2.61 (dd, J=9.3, 4.8 Hz, 4H), 2.40 (s, 3H).

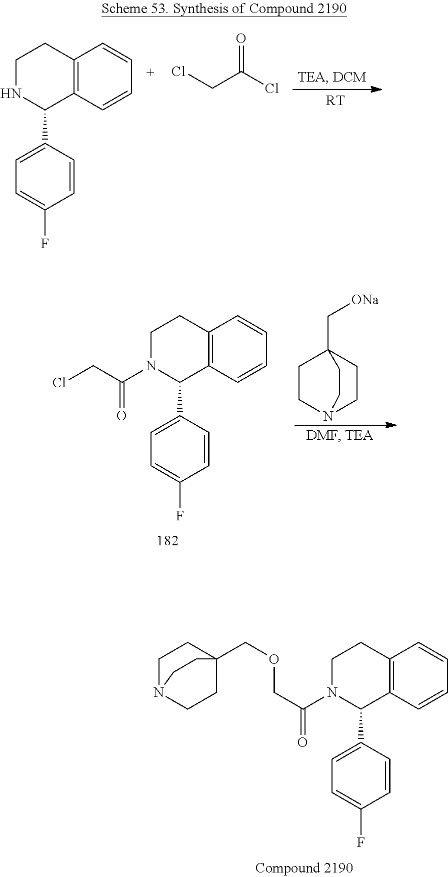

Step 1: A solution of (S)-1-(4-fluorophenyl)-1,2,3,4-tetrahydroisoquinoline 5 (2.27 g, 10 mmol) in DCM (100 mL) was sequentially treated dropwise with 2-chloroacetyl chloride (1.2 g, 11 mmol) and TEA (3 g, 30 mmol). The reaction was stirred at room temperature until TLC analysis indicated the total consumption of the starting material. The solvent was evaporated to give crude (S)-2-chloro-1-(1-(4-fluorophenyl)-3,4-dihydroisoquinolin-2(1H)-yl)ethanone (3.96 g) as a yellow solid.

Step 2A solution of quinuclidin-4-ylmethanol (1.41 g, 10 mmol), in tetrahydrofuran (50 mL) was cooled to 0 C° then NaH (4.8 g, 60% in mineral oil) was added. The reaction was stirred at room temperature for 2 hours and then concentrated under in vacuo to give crude sodium quinuclidin-4-ylmethanolate (2.32) g as a gray solid.

Step 3A solution of sodium quinuclidin-4-ylmethanolate (2.32 g, 14.2 mmol), (S)-2-chloro-1-(1-(4-fluorophenyl)-3,4-dihydroisoquinolin-2(1H)-yl)ethanone (3.96 g, 13 mmol) and TEA (3.03 g, 30 mmol) in DMF (25 mL) was stirred at 60° C. until TLC analysis indicated the total consumption of the starting material. The solution was purified directly by prep-HPLC to give (S)-1-(1-(4-fluorophenyl)-3,4-dihydroisoquinolin-2(1H)-yl)-2-(quinuclidin-4-ylmethoxy)ethenone compound 2190.

Compound 2190: LCMS: (M+H)⁺ 409; purity 100% (UV 214 nm); Retention time 1.284 min. by CP Method C

¹H NMR (400 MHz, CDCl₃) δ 7.15-6.97 (m, 5H), 6.96-6.78 (m, 3H), 6.61 (s, 1H), 4.22-3.48 (m, 8H), 3.36 (s, 1H), 3.19 (s, 2H), 3.01 (s, 1H), 2.69 (d, J=15.9 Hz, 1H), 1.82 (d, J=97.3 Hz, 6H), 1.18 (s, 1H).

The following compound was prepared using General Procedure GP-1:

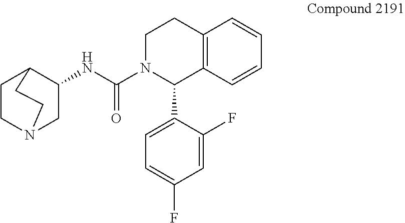 [SMILES: O=C(N[C@@H]1CN2CCC1CC2)N1CCc2ccccc2[C@@H]1c1ccc(F)cc1F]

Compound 2191 was prepared following the General CP Method for urea synthesis.

Compound 2191: LCMS: (M+H)⁺ 398.1; purity 100% (214 nm); retention time 1.505 min. by CP Method C

¹H NMR (400 MHz, CDCl₃) δ 7.25-7.15 (m, 3H), 7.03-6.96 (m, 2H), 6.87-6.76 (m, 2H), 6.50 (s, 1H), 6.26 (s, 1H), 4.22-4.12 (m, 2H), 3.37-3.30 (m, 4H), 3.11-2.99 (m, 4H), 2.84 (d, J=16 Hz, 1H), 2.26 (s, 1H), 2.07 (s, 1H), 1.92 (s, 1H), 1.71 (s, 1H).

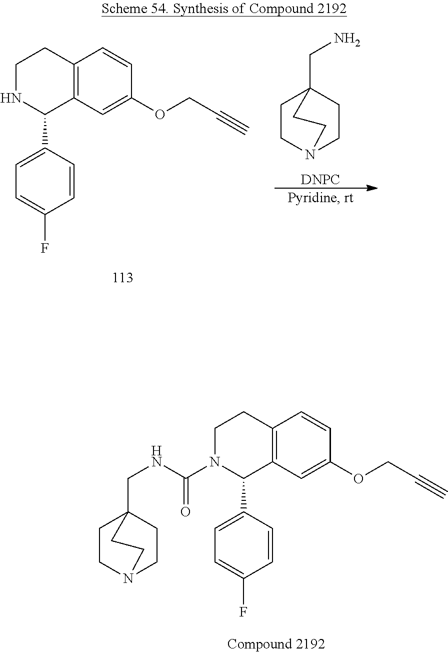

Treatment of quinuclidin-4-ylmethanamine (16.45 mg, 0.117 mmol) and (S)-1-(4-fluorophenyl)-7-(prop-2-yn-1-yloxy)-1,2,3,4-tetrahydroisoquinoline 113 (33 mg. 0.117 mmol) according to the procedure for Compound 2115, gave (S)-1-(4-fluorophenyl)-7-(prop-2-yn-1-yloxy)-N-(quinuclidin-4-ylmethyl)-3,4-dihydroisoquinoline-2(1H)-carboxamide, Compound 2192.

Compound 2192: LCMS: 100%, RT 2.594 min., (M+H)⁺ 448 (method C).

¹H NMR (400 MHz, Chloroform-d) δ 7.25-7.18 (m, 2H), 7.12 (d, J=8.4 Hz, 1H), 7.02-6.92 (m, 2H), 6.87 (dd, J=8.4, 2.6 Hz, 1H), 6.77 (d, J=2.6 Hz, 1H), 6.32 (s, 1H), 4.64 (d, J=2.4 Hz, 2H), 4.46 (t, J=6.1 Hz, 1H), 3.62-3.51 (m, 2H), 3.13 (dd, J=13.7, 6.3 Hz, 1H), 3.01 (dd, J=13.6, 5.7 Hz, 1H), 2.91-2.81 (m, 7H), 2.76 (dt, J=15.6, 5.8 Hz, 1H), 2.49 (t, J=2.3 Hz, 1H), 1.35-1.23 (m, 6H).

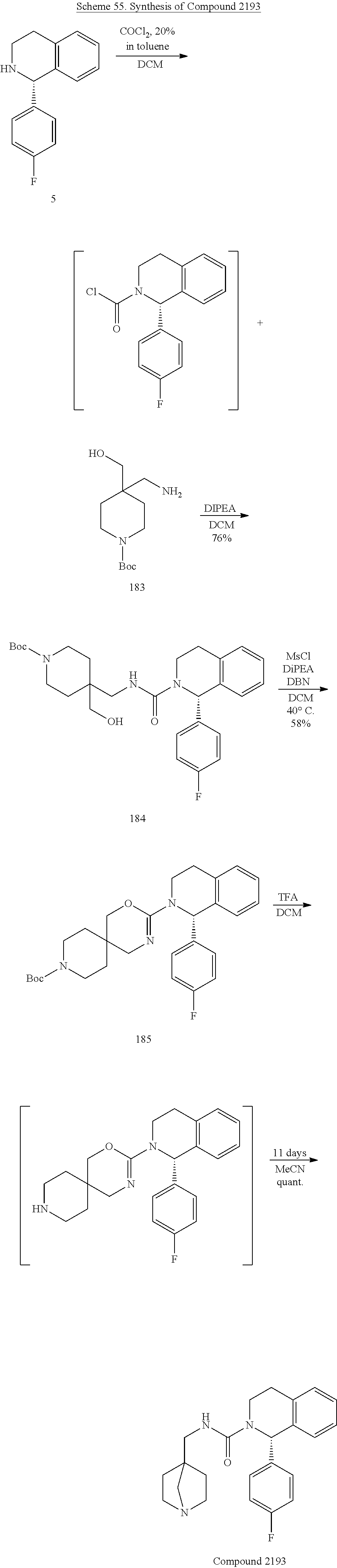

Step 1: A solution of (S)-1-(4-fluorophenyl)-1,2,3,4-tetrahydroisoquinoline 5 (100 mg, 0.440 mmol) and N,N-diisopropylethylamine (0.077 mL, 0.440 mmol) in dichloromethane (1 mL) was added dropwise to a solution of phosgene (0.255 mL, 0.484 mmol, 20% in toluene) at 0° C. The mixture was stirred for 1 hour in a closed vessel and then a mixture of tert-butyl 4-(aminomethyl)-4-(hydroxymethyl)piperidine-1-carboxylate 183 (108 mg, 0.440 mmol) and N,N-diisopropylethylamine (0.077 ml, 0.440 mmol) was added. After 10 minutes, N,N-diisopropylethylamine (0.077 ml, 0.440 mmol) was added and the mixture was allowed to stir at room temperature overnight. The reaction mixture was diluted with a mixture of heptane and ethyl acetate (1:1, 50 mL) and washed with aqueous hydrochloric acid (0.5 M, 20 mL), water (10 mL), and a mixture of brine and a saturated aqueous NaHCO₃ solution (1:1, 20 mL). The solution was dried (Na₂SO₄) and concentrated under reduced pressure. The residue was coated on an isolute cartridge and purified by flash column chromatography (silica, 40 to 100% ethyl acetate in heptane) to give tert-butyl (S)-4-((1-(4-fluorophenyl)-1,2,3,4-tetrahydroisoquinoline-2-carboxamido)methyl)-4-(hydroxymethyl)piperidine-1-carboxylate, 184 (166 mg) as a foam after co-evaporation from dichloromethane (2 mL) mixed with pentane (10 mL) and subsequently twice from pentane (10 mL). LCMS: 97%, RT 2.116 min., (M+H)⁺ 498 (method B). ¹H NMR (400 MHz, chloroform-d) δ 7.30-7.23 (m, 2H), 7.22-7.12 (m, 4H), 7.02-6.91 (m, 2H), 6.37 (s, 1H), 4.85 (t, J=6.5 Hz, 1H), 4.46 (t, J=7.2 Hz, 1H), 3.65-3.45 (m, 4H), 3.45-2.97 (m, 6H), 2.96-2.77 (m, 2H), 1.64-1.11 (m, 4H), 1.45 (s, 9H).

Step 2: Methanesulfonyl chloride (3.79 □L, 0.049 mmol) was added to a stirred solution of tert-butyl (S)-4-((1-(4-fluorophenyl)-1,2,3,4-tetrahydroisoquinoline-2-carboxamido)methyl)-4-(hydroxymethyl)piperidine-1-carboxylate 184 (22 mg, 0.044 mmol) and N,N-diisopropylethylamine (0.023 mL, 0.133 mmol) in dichloromethane (0.5 ml). After 4 days, 1,5-diazabicyclo[4.3.0]non-5-ene (0.016 ml, 0.133 mmol) was added and stirring was continued at 40° C. for 3 days. Then, the reaction mixture was diluted with dichloromethane (3 mL) and washed with a mixture of water and saturated aqueous K₂CO₃ solution (3:1). The organic phase was passed through a phase separator and concentrated to dryness under reduced pressure. The residue was purified by flash column chromatography (silica, 0-10% 7 M ammonia in methanol) in dichloromethane) to give tert-butyl (S)-3-(1-(4-fluorophenyl)-3,4-dihydroisoquinolin-2(1H)-yl)-2-oxa-4,9-diazaspiro[5.5]undec-3-ene-9-carboxylate, 185 (12 mg) as a colorless oil. LCMS: 99%, RT 2.419 min., (M+H)⁺ 480 (method B). ¹H NMR (400 MHz, Chloroform-d) δ 7.24-7.09 (m, 5H), 7.07-6.98 (m, 1H), 6.98-6.87 (m, 2H), 6.30 (s, 1H), 3.98-3.87 (m, 3H), 3.57-3.44 (m, 2H), 3.36-3.23 (m, 4H), 3.22-3.11 (m, 1H), 3.04-2.92 (m, 1H), 2.69 (dt, J=16.2, 4.0 Hz, 1H), 1.51-1.00 (m, 4H), 1.46 (s, 9H).

Step 3: Trifluoroacetic acid (0.3 mL, 3.89 mmol) was added to a stirred solution of tert-butyl (S)-3-(1-(4-fluorophenyl)-3,4-dihydroisoquinolin-2(1H)-yl)-2-oxa-4,9-diazaspiro[5.5]undec-3-ene-9-carboxylate (10 mg, 0.021 mmol) in dichloromethane (1 mL). After 1 hour, the reaction mixture was concentrated under reduced pressure and coevaporated three times from dichloromethane (2 mL).

The residue was dissolved in methanol, brought onto an SCX-2 column (1 g) and eluted with methanol until neutral. Next, the column was eluted with ammonia in methanol (1 M). The basic fraction was concentrated and taken up into acetonitrile (4 mL). After 11 days, the reaction mixture was concentrated under reduced pressure and lyophilized from acetonitrile/water to give (S)—N-((1-azabicyclo[2.2.1]heptan-4-yl)methyl)-1-(4-fluorophenyl)-3,4-dihydroisoquinoline-2(1H)-carboxamide compound 2193.

Compound 2193: LCMS: 98%, RT 2.731 min., (M+H)⁺ 380 (method C).

¹H NMR (400 MHz, chloroform-d) δ 7.26-7.14 (m, 6H), 7.02-6.92 (m, 2H), 6.32 (s, 1H), 4.55 (t, J=5.5 Hz, 1H), 3.72-3.51 (m, 4H), 3.01-2.76 (m, 4H), 2.65-2.51 (m, 2H), 2.24 (s, 2H), 1.59-1.40 (m, 2H), 1.23-1.09 (m, 2H).

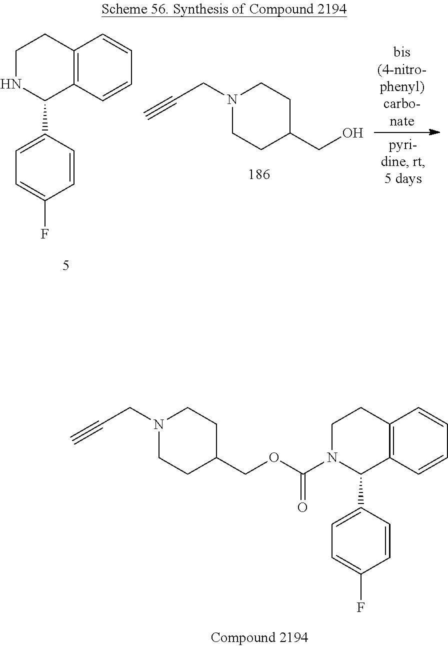

A solution of (1-(prop-2-yn-1-yl)piperidin-4-yl)methanol 186 (100 mg, 0.653 mmol) and bis(4-nitrophenyl) carbonate (199 mg, 0.653 mmol) in pyridine (4 mL) was stirred at room temperature for 4 days. After full conversion towards the intermediate carbonate was observed, (S)-1-(4-fluorophenyl)-1,2,3,4-tetrahydroisoquinoline 5 (148 mg, 0.653 mmol) was added and the mixture stirred at room temperature overnight. The mixture was concentrated under reduced pressure and the residue purified by flash column chromatography (silica, 0 to 100% ethyl acetate in pentane) and basic preparative MPLC (Linear Gradient: t=0 min 5% A, t=2 min 30% A, t=17 min 70% A; t=18 min 100% A; t=23 min 100%; detection: 215/265/285 nm) to give (1-(prop-2-yn-1-yl)piperidin-4-yl)methyl (S)-1-(4-fluorophenyl)-3,4-dihydroisoquinoline-2(1H)-carboxylate Compound 2194.

Compound 2194: LCMS: 99%, RT 2.98 min., (M+H)⁺ 407 (method C).

¹H NMR (400 MHz, Chloroform-d) mixture of rotamers δ 7.24-7.08 (m, 5H), 7.06-7.00 (m, 1H), 6.99-6.91 (m, 2H), 6.53-6.12 (m, 1H), 4.21-3.91 (m, 3H), 3.31 (d, J=2.4 Hz, 2H), 3.27-3.16 (m, 1H), 3.05-2.87 (m, 3H), 2.76 (dt, J=16.1, 4.0 Hz, 1H), 2.29-2.15 (m, 3H), 1.79-1.62 (m, 3H), 1.47-1.33 (m, 2H).

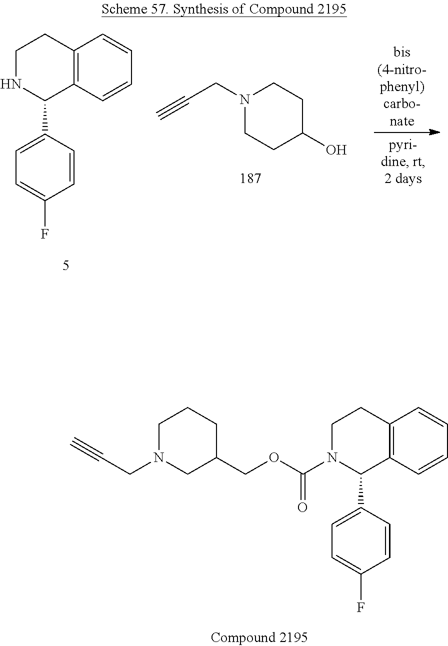

Treatment of 1-(prop-2-yn-1-yl)piperidin-4-ol 187 (100 mg, 0.718 mmol) and (S)-1-(4-fluorophenyl)-1,2,3,4-tetrahydroisoquinoline (163 mg, 0.718 mmol) according to the procedure for Compound 2194, gave 1-(prop-2-yn-1-yl)piperidin-4-yl (S)-1-(4-fluorophenyl)-3,4-dihydroisoquinoline-2(1H)-carboxylate Compound 2195.

Compound 2195 LCMS: 96%, RT 2.95 min., (M+H)⁺ 393 (method C). ¹H NMR (400 MHz, Chloroform-d) mixture of rotamers δ 7.24-7.15 (m, 5H), 7.08-7.00 (m, 1H), 7.00-6.92 (m, 2H), 6.51-6.13 (m, 1H), 4.85-4.69 (m, 1H), 4.27-3.92 (m, 1H), 3.32 (d, J=2.5 Hz, 2H), 3.28-3.16 (m. 1H), 3.05-2.90 (m, 1H), 2.86-2.67 (m, 3H), 2.55-2.40 (m, 2H), 2.25 (t, J=2.4 Hz, 1H), 2.08-1.90 (m, 2H), 1.90-1.70 (m, 2H).

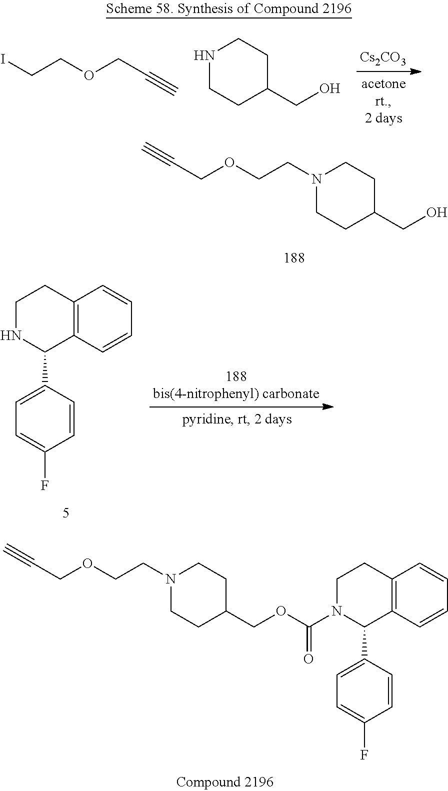

Step 1: Cesium carbonate (236 mg, 0.724 mmol) and 3-(2-iodoethoxy)prop-1-yne (152 mg, 0.724 mmol) were sequentially added to a solution of piperidin-4-ylmethanol (83.4 mg, 0.724 mmol) in acetone (5 mL). The mixture was stirred at room temperature for 2 days. The solids were filtered off and the filtrate was concentrated under reduced pressure to give (1-(2-(prop-2-yn-1-yloxy)ethyl)piperidin-4-yl)methanol 188 (36 mg) as an off-white solid. ¹H NMR (300 MHz, Chloroform-d) δ 4.17 (d, J=2.4 Hz, 2H), 3.68 (t, J=5.8 Hz, 2H), 3.54-3.45 (m, 2H), 3.06-2.95 (m, 2H), 2.62 (t, J=5.8 Hz, 2H), 2.42 (t, J=2.4 Hz, 1H), 2.03 (td, J=11.6, 2.5 Hz, 2H), 1.82-1.42 (m, 4H), 1.36 (td, J=12.3, 3.8 Hz, 2H).

Step 2: Treatment of (1-(2-(prop-2-yn-1-yloxy)ethyl)piperidin-4-yl)methanol 188 (36 mg, 0.18 mmol) and (S)-1-(4-fluorophenyl)-1,2,3,4-tetrahydroisoquinoline 5 (41 mg, 0.18 mmol) according to the procedure for compound 2195, gave (1-(2-(prop-2-yn-1-yloxy)ethyl)piperidin-4-yl)methyl (S)-1-(4-fluorophenyl)-3,4-dihydroisoquinoline-2(1H)-carboxylate compound 2196.

Compound 2196: LCMS: 96%, RT 1.624 min, (M+H)⁺ 451 (method E).

¹H NMR (300 MHz, Chloroform-d) δ 7.19 (tdd, J=8.9, 6.3, 2.0 Hz, 5H), 7.07-6.88 (m, 3H), 6.38 (s, 1H), 4.17 (d, J=2.4 Hz, 2H), 4.00 (qd, J=10.6, 6.0 Hz, 3H), 3.66 (t, J=5.7 Hz, 2H), 3.21 (ddd, J=13.2, 10.7, 4.4 Hz, 1H), 2.96 (tt, J=9.0, 4.4 Hz, 3H), 2.75 (dt, J=16.2, 4.0 Hz, 1H), 2.60 (t, J=5.8 Hz, 2H), 2.41 (t, J=2.4 Hz, 1H), 2.00 (td, J=11.7, 2.2 Hz, 2H), 1.74-1.63 (m, 3H), 1.48-1.29 (m, 2H).

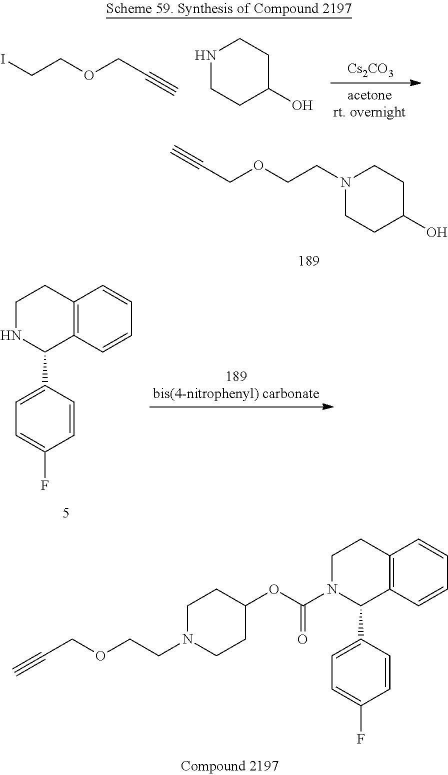

Step 1: Cesium carbonate (318 mg, 0.976 mmol) was added to a solution of piperidin-4-ol (98.7 mg, 0.976 mmol) and 3-(2-iodoethoxy)prop-1-yne (205 mg, 0.976 mmol) in acetone (3 mL). The mixture was stirred at room temperature for 18 hours. The solids were filtered off and washed with acetone and the filtrate was concentrated under reduced pressure. The residue was taken up in diethyl ether and the resulting solids were filtered off and washed with diethyl ether. The filtrate was concentrated under reduced pressure to give 1-(2-(prop-2-yn-1-yloxy)ethyl)piperidin-4-ol 189 (109 mg) as a yellow oil. ¹H NMR (300 MHz, Chloroform-d) δ 4.17 (dd, J=2.4, 0.9 Hz, 2H), 3.93-3.49 (m, 3H), 2.97-2.75 (m, 2H), 2.61 (td, J=5.7, 0.9 Hz, 2H), 2.42 (td, J=2.3, 0.9 Hz, 1H), 2.21 (ddd, J=12.0, 9.9, 3.1 Hz, 2H), 1.98-1.81 (m, 2H), 1.71-1.50 (m, 2H).

Step 2: Treatment of 1-(2-(prop-2-yn-1-yloxy)ethyl)piperidin-4-ol 189 (100 mg, 0.546 mmol) and (S)-1-(4-fluorophenyl)-1,2,3,4-tetrahydroisoquinoline 5 (124 mg, 0.546 mmol) according to the procedure for Compound 2196, gave 1-(2-(prop-2-yn-1-yloxy)ethyl)piperidin-4-yl (S)-1-(4-fluorophenyl)-3,4-dihydroisoquinoline-2(1H)-carboxylate Compound 2197

Compound 2197: LCMS: 95%, RT 1.64 min., (M+H)⁺ 437 (method E).

¹H NMR (300 MHz, Chloroform-d) mixture of rotamers δ 7.25-7.10 (m, 5H), 7.08-6.88 (m, 3H), 6.54-6.09 (m, 1H), 4.78 (sept, J=4.1 Hz, 1H), 4.27-3.89 (m, 1H) 4.18 (s, 2H), 3.67 (t, J=5.6 Hz, 2H), 3.22 (ddd, J=13.2, 10.8, 4.3 Hz, 1H), 3.08-2.88 (m, 1H), 2.87-2.67 (m, 3H), 2.62 (t, J=5.6 Hz, 2H), 2.48-2.23 (m, 2H), 2.42 (t, J=2.4 Hz, 1H) 2.05-1.89 (m, 2H) 1.88-1.68 (m, 2H).

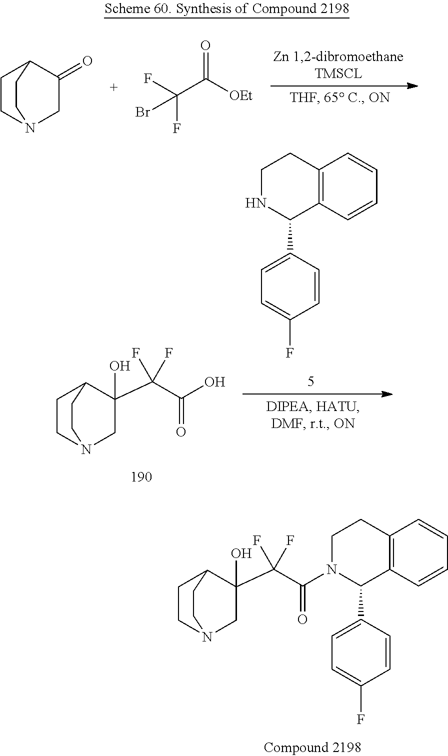 [SMILES: O=C1CN2CCC1CC2 ; CCOC(=O)C(F)(F)Br ; O=C(O)C(F)(F)C1(O)CN2CCC1CC2 ; Fc1ccc([C@@H]2NCCc3ccccc32)cc1 ; O=C(N1CCc2ccccc2[C@@H]1c1ccc(F)cc1)C(F)(F)C1(O)CN2CCC1CC2]

Step 1: Under argon atmosphere, a heat gun dried vial was loaded with activated zinc dust (0.334 g, 5.11 mmol) and additionally dried by heating with a heat gun under a stream of argon, for 5 min. The vial was allowed to reach room temperature and anhydrous tetrahydrofuran (4 ml) and 1,2-dibromoethane (0.011 ml, 0.128 mmol), were added sequentially. The reaction mixture was transferred into a pre-heated oil bath (65° C.). After stirring for 1 minute, the reaction mixture was cooled to room temperature. This warming/cooling sequence was repeated 3 times. At room temperature, trimethylsilyl chloride (0.016 ml, 0.128 mmol) was added dropwise and, after 20 minutes, ethyl 2-bromo-2,2-difluoroacetate (0.328 ml, 2.56 mmol) was added dropwise (exothermic). The reaction mixture was transferred into a pre-heated oil bath (65° C.) and after stirring for 5 minutes it was allowed to cool to room temperature. The stirring was stopped and the supernatant was added to a solution of quinuclidin-3-one (0.16 g, 1.278 mmol) in tetrahydrofuran (dry, 2 ml) under argon atmosphere. The reaction vial was transferred into a pre-heated oil bath (65° C.) and stirred overnight. After cooling to room temperature, the reaction mixture was concentrated to dryness under reduced pressure. The residue was triturated with water and the resulting solid was filtered and rinsed with water. The filtrate was concentrated under reduced pressure. The residue was purified by preparative LCMS (Method K; Gradient: t=0 min 98% A, t=2.5 min 98% A; t=15 min 60% A; t=19.5 min 60% A, post time 4.5 min) to afford 2,2-difluoro-2-(3-hydroxyquinuclidin-3-yl)acetic acid 190 (80 mg) as white solid after lyophilisation. LCMS: non-UV active, RT 0.2 min., (M+H)⁺ 222 (method A). ¹H NMR (400 MHz, Deuterium Oxide) δ 3.84-3.70 (m, 1H), 3.35-3.04 (m, 5H), 2.48-2.43 (m, 1H), 2.27-2.02 (m, 2H), 1.93-1.79 (m, 1H), 1.80-1.63 (m, 1H).

Step 2: 1-[Bis(dimethylamino)methylene]-1H-1,2,3-triazolo[4,5-b]pyridinium 3-oxid hexafluorophosphate (HATU, 0.109 g, 0.286 mmol) followed by N,N-diisopropylethylamine (0.149 mL, 0.858 mmol) was added to a suspension of 2,2-difluoro-2-(3-hydroxyquinuclidin-3-yl)acetic acid 190 (76 mg, 0.343 mmol) in anhydrous N,N-dimethylformamide (2.6 mL) under an argon atmosphere. After 15 minutes, (S)-1-(4-fluorophenyl)-1,2,3,4-tetrahydroisoquinoline 5 (65 mg, 0.286 mmol) was added and stirring was continued overnight. The reaction mixture was diluted with ethyl acetate and quenched with a saturated aqueous solution of NaHCO₃. The layers were separated and the aqueous phase was extracted with three 10 mL portions of ethyl acetate. The combined organic phase was dried (Na₂SO₄) and concentrated under reduced pressure. The residue was purified by flash column chromatography (silica, 0 to 5% methanol in chloroform) to give of 2,2-difluoro-1-((S)-1-(4-fluorophenyl)-3,4-dihydroisoquinolin-2(1H)-yl)-2-(3-hydroxyquinuclidin-3-yl)ethan-1-one Compound 2198. Compound 2198: LCMS: 98.2%, RT 4.14 min., (M+H)⁺ 431 (method D).

¹H NMR (400 MHz, Chloroform-d+Deuterium Oxide) δ 7.34-7.20 (m, 3H), 7.20-7.12 (m, 2H), 7.11-6.94 (m, 3H), 6.77 (d, J=5.8 Hz, 1H), 4.32-4.12 (m, 1H), 3.52-3.31 (m, 2H), 3.27-2.94 (m, 6H), 2.93-2.84 (m, 1H), 2.55-2.46 (m, 1H), 2.42-2.28 (m, 1H), 2.15-2.01 (m, 1H), 1.76-1.64 (m, 1H), 1.61-1.44 (m, 1H).

Synthesis of COMPOUND 2202, COMPOUND 2203 and COMPOUND 2204

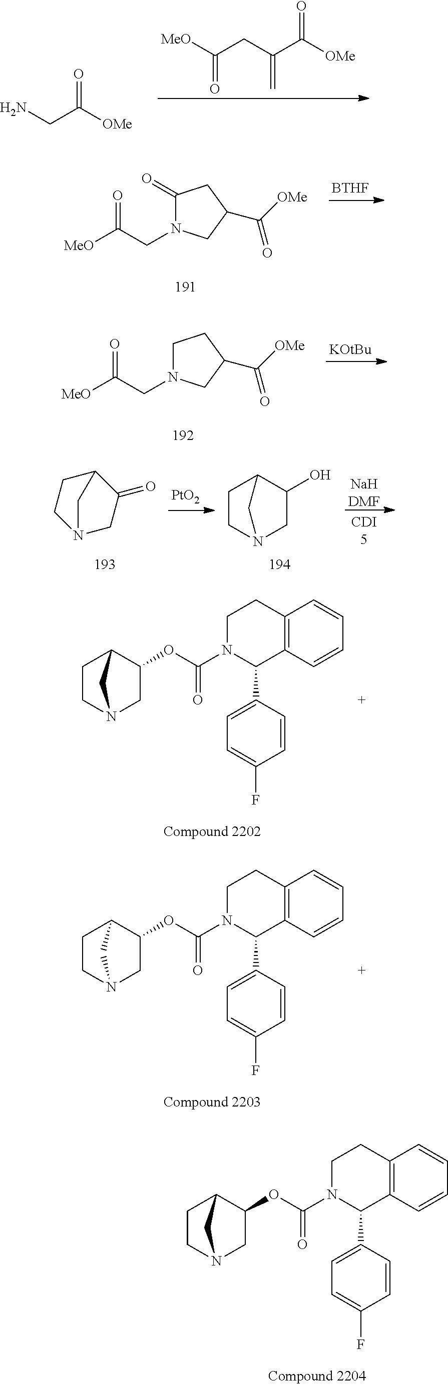

Step 1: To a mixture of sodium methoxide (2.00 g, 37.9 mmol) and methanol (9 mL) at 0° C. were added glycine methyl ester hydrochloride (4.76 g, 37.9 mmol) and dimethyl itaconate (5.00 g, 31.6 mmol.) The reaction mixture was heated at reflux for 16 hours before cooling to room temperature. The solid was collected by filtration, washed with dichloromethane, and filtrate concentrated. The residue was diluted with 5N HCl (50 mL) and extracted with dichloromethane (4×50 mL). The combined organic layers were dried over MgSO₄, filtered, and concentrated in vacuo. The crude product was used directly without further purification.

LCMS: (M+H)⁺=216; Retention time=1.293 min. LCMS CP Method B

Step 2: To a solution of methyl 1-(2-methoxy-2-oxoethyl)-5-oxopyrrolidine-3-carboxylate (3.40 g, 16.0 mmol) in THF (20 mL) at 0° C. was added a solution of Borane in THF (1.0M/THF) (32.0 mL, 32.0 mmol). The reaction mixture was heated at reflux for 1 hour and then cooled to room temperature and allowed to stir an additional 12 hours. The reaction mixture was quenched by adding a saturated solution of potassium carbonate (5.52 g in 20 mL H₂O) and heating at reflux for an additional hour. The solvent was removed in vacuo and the residue was treated with 5N HCl (25 mL). The aqueous layer was washed with dichloromethane (2×30 mL), made alkaline by the addition of solid potassium carbonate and extracted with dichloromethane (5×30 mL). The combined organic extracts were dried over MgSO₄, filtered and concentrated in vacuo to give the product.

LCMS: (M+H)⁺=202; Retention time=1.060 min. LCMS CP Method B

Step 3: To a refluxing solution of potassium tert-butoxide (2.46 g, 22.0 mmol) in toluene (32 mL) was added dropwise a solution of methyl 1-(2-methoxy-2-oxoethyl)pyrrolidine-3-carboxylate (2.00 g, 10.0 mmol) in toluene (10 mL) over 1 hour and the reaction mixture was stirred for additional 3 hours at reflux. The stirred room temperature reaction mixture was cooled to −10° C. and treated with Acetic acid (1.3 mL) and the toluene layer was extracted with 5N HCl (4×50 mL). The combined acidic aqueous layer was heated to 50° C. for 8 hours before concentration to half volume in vacuo. The reaction mixture was made basic by the addition of solid potassium carbonate and extracted with dichloromethane (5×50 mL). The combined organic layers were concentrated in vacuo and triturated with diethyl ether. After removal of the solid by filtration the solution was concentrated to give the product.

LCMS: (M+H)⁺=114; Retention time=0.987 min. LCMS CP Method B

Step 4: To a solution of 1-azabicyclo [2.2.1] heptan-3-one (0.30 g, 2.7 mmol) in ethanol (2-3 mL) was added PtO₂ (100 mg, 50 wt %) and reaction mixture was stirred at room temperature for 4 h under a Hydrogen atmosphere. The catalyst was filtered off, the filter cake washed with ethanol and the organic layer concentrated under reduced pressure to give the desired product.

LCMS: (M+H)⁺=113; Retention time=0.876 min. LCMS CP Method B

Step 5: To a suspension of (S)-1-(4-fluorophenyl)-1,2,3,4-tetrahydroiso-quinoline (477 mg, 2.0 mmol) and NaH (120 mg, 4.2 mmol) in DMF (5 mL) at 0° C. was added CDI (324 mg, 2.0 mmol). The reaction mixture was stirred at room temperature for 30 min. and then 1-azabicyclo[2.2.1]heptan-3-ol (226 mg, 2.0 mmol) was introduced and the resulting mixture heated to 60° C. for 6 h. The reaction mixture was diluted with water (50 mL), extracted with EA (3×30 mL) and the combined organic layer dried over anhydrous Na₂SO₄. After removal of the drying agent, the filtrate was concentrated to give a crude oil which was purified by Prep-HPLC to afford the product.

The diastereomers were separated by chiral SFC eluting with CO₂/MeOH containing 0.2% MA (75%:25%, MA=7.0M methanolic ammonia) over an EnantioPak® OX-H column (4.6*100 mm 5 μm) to give COMPOUND 2202 (retention time=1.473 min), COMPOUND 2203 (retention time=1.495 min), COMPOUND 2204 (retention time=2.413 min). Stereochemical assignment at 1 position of the tetrahydroisoquinoline is based on enantiomerically pure starting materials of known configuration. The stereochemical assignment at the piperidine chiral center is assigned arbitrarily based on chromatographic elution order in comparison to related analogues of known configuration.

COMPOUND 2202: LCMS: (M+H)⁺=366; (214 nm); Retention time=1.815 min. LCMS CP Method Chiral SFC: CO₂/MeOH containing 0.2% ammonia over CHIRALPAK® IG column (4.6*100 mm 5 μm), retention time=1.473 min).

COMPOUND 2203: LCMS: (M+H)⁺=366; (214 nm); Retention time=1.837 min. LCMS CP Method C

Chiral SFC: CO₂/MeOH containing 0.2% ammonia over CHIRALPAK® IG column (4.6*100 mm 5 μm), retention time=1.495 min).

COMPOUND 2204: LCMS: (M+H)⁺=366; (214 nm); Retention time=1.811 min. LCMS CP Method C

Chiral SFC: CO₂/MeOH containing 0.2% ammonia over CHIRALPAK® IG column (4.6*100 mm 5 μm), retention time=2.413 min).

Synthesis of COMPOUND 2229 and COMPOUND 2228

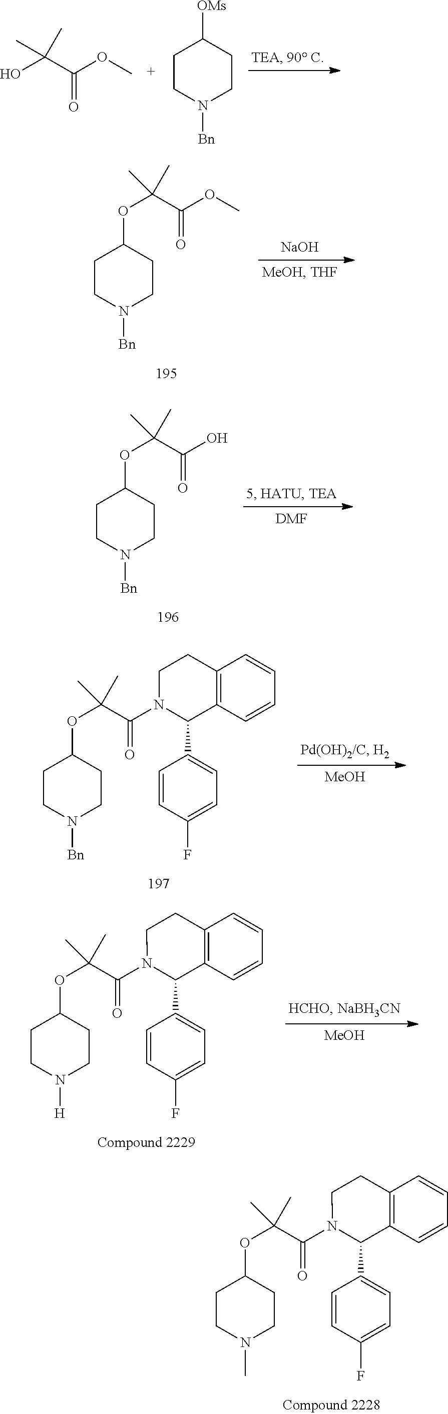

Step 1: A mixture of methyl 2-hydroxy-2-methylpropanoate (2.0 g, 7.4 mmol), 1-benzylpiperidin-4-yl methanesulfonate (5.3 g, 44.5 mmol) and TEA (1 mL, 7.4 mmol) was stirred at 90° C. for 2 h. The reaction mixture was concentrated in vacuo and the residue purified by column chromatography to give methyl 2-(1-benzylpiperidin-4-yloxy)-2-methylpropanoate.

LCMS: (M+H)⁺=292; Retention time=1.178 min. LCMS CP Method E

Step 2: NaOH (137 mg, 3.4 mmol) was added to the mixture of methyl 2-(1-benzylpiperidin-4-yloxy)-2-methylpropanoate (200 mg, 0.7 mmol) in MeOH (0.7 mL), H₂O (0.7 mL) and THF (1.4 mL) at 0° C. The reaction mixture was warmed to ambient temperature and stirred for 2 h., concentrated and the pH adjusted to 3 by the addition of 1N HCl. After complete solvent removal, the residue was triturated with MeOH (5 mL), filtered to remove solids and concentrated in vacuo to give 2-(1-benzylpiperidin-4-yloxy)-2-methylpropanoic acid (100 mg) as a white solid.

LCMS: (M+H)⁺=278; Retention time=1.116 min. LCMS CP Method E

Step 3: To a solution of 2-(1-benzylpiperidin-4-yloxy)-2-methylpropanoic acid (100 mg, 0.36 mmol) in DMF (3 mL) were added (S)-1-(4-fluorophenyl)-1,2,3,4-tetrahydroiso-quinoline (68 mg, 0.3 mmol), HATU (137 mg, 0.36 mmol) and TEA (0.1 mL, 0.72 mmol) at 0° C. The reaction mixture was stirred at ambient temperature for 2 h., diluted with ethyl acetate (50 mL) and water (30 mL). The aqueous layer was extracted with ethyl acetate (3×30 mL) and the combined organic layer was washed with brine (30 mL), dried over Na₂SO₄ and concentrated in vacuo. The residue was purified by column chromatography to afford (S)-2-(1-benzylpiperidin-4-yloxy)-1-(1-(4-fluorophen-yl)-3,4-dihydroisoquinolin-2(1H)-yl)-2-methylpropan-1-one.

LCMS: (M+H)⁺=487; Retention time=1.654 min. LCMS CP Method F

Step 4: To a solution of (S)-2-(1-benzylpiperidin-4-yloxy)-1-(1-(4-fluorophenyl)-3,4-dihydroisoquinolin-2(1H-y-1)-2-methylpropan-1-one (80 mg, 0.16 mmol) in MeOH (5 mL) was added Pd(OH)₂ (20 mg, 10%/C). The reaction mixture was stirred at room temperature overnight under Hydrogen atmosphere, filtrated and the filtrate concentrated under reduced pressure. The residue was purified by Prep-HPLC to give (S)-1-(1-(4-fluorophenyl)-3,4-dihydroisoquinolin-2(1H)-yl)-2-methyl-2-(piperidin-4-yloxy)propan-1-one COMPOUND 2229.

COMPOUND 2229: LCMS: (M+H)⁺=397 (214 nm); retention time=1.530 min. LCMS CP Method F

Chiral SFC: CO₂/MeOH containing 0.2% MA (70%:30%) over a CHIRALPAK® IG column (4.6*100 mm 5 μm), retention time=2.252 min.

Step 5: To a solution of (S)-1-(1-(4-fluorophenyl)-3,4-dihydroisoquinolin-2(1H)-yl)-2-methyl-2-(piperidin-4-yloxy)propan-1-one (30 mg, 0.075 mmol) in MeOH (4 mL) were added 37% HCHO (12 mg, 0.15 mmol) and NaBH₃CN (10 mg, 0.15 mmol) at 0° C. The mixture was stirred at room temperature for 2 h and then concentrated under reduced pressure. Purification of the residue by Prep-HPLC afforded (S)-1-(1-(4-fluorophenyl(-3,4-dihydroisoquinolin-2(1H)-yl)-2-methyl-2-(1-methylpiperidin-4-yloxy)propan-1-one COMPOUND 2228.

COMPOUND 2228: LCMS: (M+H)⁺=411; (214 nm); retention time=1.526 min. LCMS CP Method F

Chiral SFC: CO₂/MeOH containing 0.2% MA (75%:25%) over a CHIRALPAK® IG column (4.6*100 mm 5 μm), retention time=2.381 min.

Compound 2247 was prepared using a similar synthesis as Compounds 2202-2204

COMPOUND 2247: LCMS: (M+H)⁺=409; retention time=1.526 min. Method F

Synthesis of COMPOUND 2211, COMPOUND 2212, COMPOUND 2213 and COMPOUND 2214

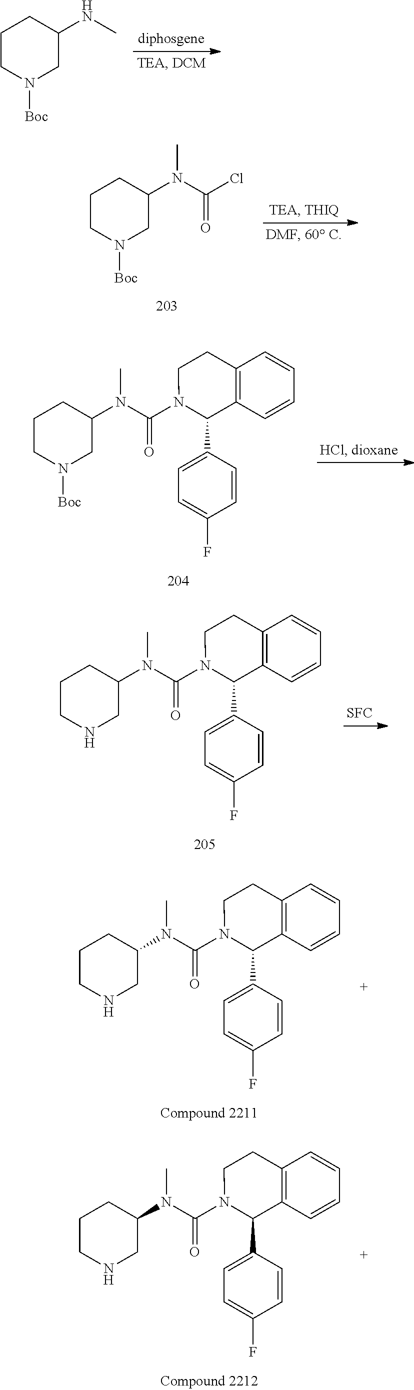

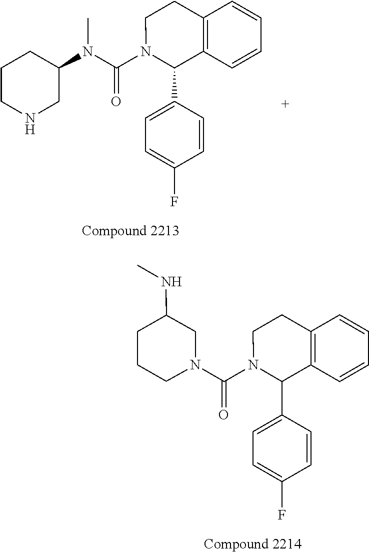 [SMILES: CN(C(=O)N1CCc2ccccc2[C@@H]1c1ccc(F)cc1)[C@@H]1CCCNC1 ; CNC1CCCN(C(=O)N2CCc3ccccc3C2c2ccc(F)cc2)C1]

Step 1: To a solution of tert-butyl 3-(methylamino)piperidine-1-carboxylate (300 mg, 1.4 mmol) in DCM (4 mL) was added TEA (0.4 mL, 2.8 mmol) at 0° C. The resulting reaction mixture was stirred 10 min before the introduction of diphosgene (0.14 mL, 1.1 mmol) at 0° C. The reaction mixture was stirred at the 0° C. for 2 h before concentration under reduced pressure to give the crude tert-butyl 3-(chlorocarbonyl(methyl)amino)piperidi-ne-1-carboxylate which was used directly in the next step reaction without further purification.

Step 2: To a solution of (S)-1-(4-fluorophenyl)-1,2,3,4-tetrahydroisoquinoline (200 mg, 0.9 mmol) and TEA (0.6 mL, 4.4 mmol) in DMF (2 mL) was added a solution of tert-butyl 3-(chlorocarbonyl(methyl)amino)piperidine-1-carboxylate (320 mg, 1.2 mmol) in DMF (2 mL). The reaction mixture was heated to 60° C. for 2 h. before dilution with ethyl acetate (60 mL) and water (20 mL). The aqueous layer was extracted with ethyl acetate (3×30 mL) and the combined organic phase were washed with brine (30 mL), dried over Na₂SO₄ and filtered. The filtrate was concentrated under reduced pressure to give a residue which was purified by column chromatography to give tert-butyl-3-((S)-1-(4-fluorophenyl)-N-methyl-1,2,3,4-tetrahydroisoquinoline-2-carboxamido)-piperidine-1-carboxylate.

LCMS: (M−55)⁺=412; Retention time=2.271 min. LCMS CP Method E

Step 3: To a solution of tert-butyl 3-((S)-1-(4-fluorophenyl)-N-methyl-1,2,3,4-tetrahydroisoquinoline-2-carboxamido)piperidine-1-carboxylate (200 mg, 0.4 mmol) in dioxane (2 mL) was added HCl in dioxane (4N, 2 mL) at 0° C. The reaction mixture was stirred at room temperature for 2 h and then concentrated under reduced pressure to give a residue which was purified by Prep-HPLC to give (1S)-1-(4-fluorophenyl)-N-methyl-N-(piperidin-3-yl)-3,4-dihydroisoquinoline-2(1H)-carboxamide.

LCMS: (M+H)⁺=368; Retention time=1.725 min. LCMS CP Method F

The diastereomers were separated by chiral HPLC eluting with n-Hexane (0.1% DEA):EtOH (0.1% DEA)=70:30 over an EnantioPak® IC column (4.6*250 mm 5 μm) to give COMPOUND 2211 (retention time=7.236 min), COMPOUND 2212 (retention time=9.236 min), COMPOUND 2213 (9.5 mg, retention time=11.073 min) and COMPOUND 2214 (retention time=8.069 min). Stereochemistry is arbitrarily assigned based on chromatographic elution order as compared to related analogues of known configuration.

COMPOUND 2211: LCMS: (M+H)⁺=368; (214 nm); retention time=1.509 min. LCMS CP Method F

Chiral SFC: CO₂/MeOH containing 0.2% methanolic ammonia over a CHIRALPAK® IG column (4.6*100 mm 5 μm), retention time=12.371 min.

COMPOUND 2212: LCMS: (M+H)⁺=368 (214 nm); retention time=1.504 min. LCMS CP Method F

Chiral SFC: CO₂/MeOH containing 0.2% methanolic ammonia over a CHIRALPAK® IG column (4.6*100 mm 5 μm), retention time=16.517 min.

COMPOUND 2213: LCMS: (M+H)⁺=368; (214 nm); retention time=1.505 min. LCMS CP Method F

Chiral SFC: CO₂/MeOH containing 0.2% methanolic ammonia over a CHIRALPAK® IG column (4.6*100 mm 5 μm), retention time=18.277 min.

COMPOUND 2214: LCMS: (M+H)⁺=368; (214 nm); retention time=1.505 min. LCMS CP Method F

Chiral SFC: CO₂/MeOH containing 0.2% methanolic ammonia over a CHIRALPAK® IG column (4.6*100 mm 5 μm), retention time=18.705 min.

Synthesis of COMPOUND 2215

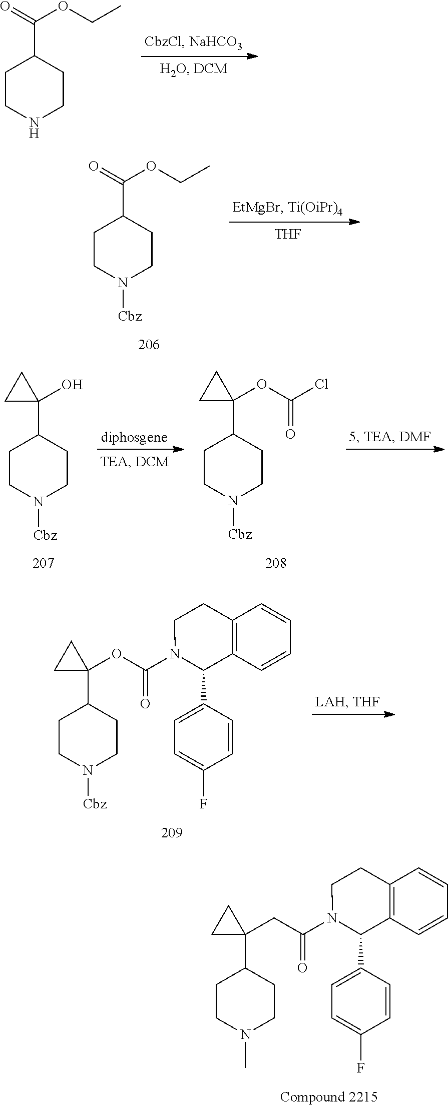

Step 1: To a solution of ethyl piperidine-4-carboxylate (2.0 g, 12.7 mmol) in H₂O (25 mL) and DCM (25 mL) were added NaHCO₃ (2.14 g, 25.5 mmol) and CbzCl (2.2 mL, 15.3 mmol) at 0° C. The reaction mixture was stirred at room temperature overnight and then extracted with DCM (3×60 mL). The combined organic phase was washed with brine (40 mL), dried over Na₂SO₄, filtered and concentrated. The residue was purified by column chromatography to give 1-benzyl 4-ethyl piperidine-1,4-dicarboxylate.

LCMS: (M+H)⁺=292; Retention time=2.034 min. LCMS CP Method F

Step 2: To a solution of 1-benzyl 4-ethyl piperidine-1,4-dicarboxylate (1.5 g, 5.2 mmol) and Ti(OⁱPr)₄ (3 mL, 10.3 mmol) in THF (25 mL) was added EtMgBr (15.5 mL, 15.5 mmol) dropwise at 0° C. over 30 min. The mixture was stirred at room temperature overnight. The reaction mixture was quenched with NH₄Cl (20 mL) and the aqueous layer extracted with ethyl acetate (3×50 mL). The combined organic layers were washed with brine (30 mL), dried over Na₂SO₄, filtered and concentrated. The residue was purified by column chromatography to give benzyl 4-(1-hydroxy-cyclopropyl)piperidine-1-carboxylate.

LCMS: (M+H)⁺=276; Retention time=1.708 min. LCMS CP Method E

Step 3: To the solution of benzyl 4-(1-hydroxy-cyclopropyl)piperidine-1-carboxylate (350 mg, 1.3 mmol) in DCM (4 mL) was added TEA (0.35 mL, 2.5 mmol) at 0° C. The mixture was stirred for 30 min and then diphosgene (0.2 mL, 1.6 mmol) was added. The reaction mixture was stirred for 2 h and then concentrated to give benzyl 4-(1-(chlorocarbonyloxy)cyclopropyl)piperidine-1-carboxylate which was used directly without further purification.

Step 4: To a solution of (S)-1-(4-fluorophenyl)-1,2,3,4-tetrahydroisoquinoline (170 mg, 0.7 mmol) and TEA (0.52 mL, 3.7 mmol) in DMF (2 mL) was added a solution of benzyl 4-(1-(chlorocarbonyloxy)cyclopropyl)piperidine-1-carboxylate (380 mg, 1.1 mmol) in DMF (2 mL). The reaction mixture was stirred at room temperature for 1 h and then diluted with ethyl acetate (40 mL) and water (20 mL). The mixture was extracted with ethyl acetate (3×50 mL). The combined organic phase was washed with brine (30 mL), dried over Na₂SO₄, filtered and concentrated under reduced pressure. The residue was purified by column chromatography to give of (S)-1-(1-(benzyloxycarbonyl)(piperidin-4-yl)cyclopropyl-1-(4-fluorophenyl)-3,4-dihydroisoquinoline-2(1H)carboxylate.

LCMS: (M+H)⁺=529; Retention time=2.469 min. LCMS CP Method F

Step 5: To the solution of (S)-1-(1-(benzyloxycarbonyl)-piperidin-4-yl)cyclopropyl 1-(4-fluorophenyl)-3,4-dihydroisoquin-oline-2(1H-carboxylate (201 mg, 1 mmol) in THF (4 mL) was added a solution of LAH in THF (1N, 1.1 mL, 1.1 mmol) at 0° C. The mixture was stirred at room temperature for 2 h and then quenched with 10 mL saturated NH₄Cl aqueous solution (10 mL). The aqueous layer was extracted with ethyl acetate (3×40 mL) and the combined organic layers were washed with brine (30 mL), dried over Na₂SO₄, filtered and concentrated under reduced pressure. The residue was purified by Prep-HPLC to give (S)-1-(1-methylpiperidin-4-yl)cyclopropyl 1-(4-fluorophenyl)-3,4-dihydroiso-quinoline-2(1H-carboxylate COMPOUND 2215.

COMPOUND 2215: LCMS: (M+H)⁺=409; (214 nm); retention time=1.585 min. LCMS CP Method F

Chiral SFC: CO₂/MeOH containing 0.2% methanolic ammonia over a CHIRALPAK® IG column (4.6*100 mm 5 μm), retention time=2.071 min.

Synthesis of COMPOUND 2222

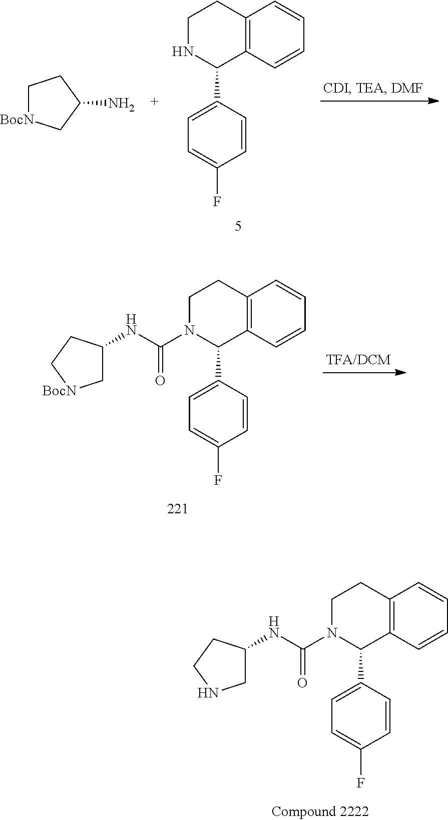 [SMILES: CC(C)(C)OC(=O)N1CC[C@H](N)C1 ; Fc1ccc([C@@H]2NCCc3ccccc32)cc1 ; CC(C)(C)OC(=O)N1CC[C@H](NC(=O)N2CCc3ccccc3[C@@H]2c2ccc(F)cc2)C1 ; O=C(N[C@H]1CCNC1)N1CCc2ccccc2[C@@H]1c1ccc(F)cc1]

Step 1: A solution of (S)-1-(4-fluorophenyl)-1, 2, 3, 4-tetrahydroisoquinoline (454 mg, 2 mmol), TEA (404 mg, 4 mmol), (S)-tert-butyl 3-aminopyrrolidine-1-carboxylate (372 mg, 2 mmol) and CDI (486 mg, 3 mmol) in DMF (5 mL) was stirred at 60° C. for 3 h. Then it was diluted with water (50 mL) and extracted with EA (3×50 mL). The combined organic layers were washed with brine (50 mL), dried over anhydrous Na₂SO₄, filtered and concentrated to give a light yellow oil which was purified by Prep-HPLC to give (S)-tert-butyl 3-((S)-1-(4-fluorophenyl)-1, 2, 3, 4-tetrahydroisoquinoline-2-carboxamido)pyrrolidine-1-carboxylate.

LCMS: CP-0024112-075, (214 nm); Retention time=1.254 min. Method C1

Step 2: To a solution of (S)-tert-butyl 3-((S)-1-(4-fluorophenyl)-1, 2, 3, 4-tetrahydroisoquinoline-2-carboxamido)pyrrolidine-1-carboxylate (120 mg, 0.27 mmol) in DCM (2 mL) was added TFA (0.2 mL) and the resulting mixture was stirred at room temperature for 1 h. After concentration in vacuo, the residue was purified by Prep-HPLC to give (S)-1-(1-(4-fluorophenyl)-3, 4-dihydroisoquinolin-2(1H)-yl)-3-(4-methylpiperazin-1-yl) propane-1, 3-dione.

LCMS: CP-0024112-078, (214 nm); Retention time=1.381 min. Method C1

COMPOUND 2222: LCMS: (M+H)⁺=340; (214 nm); Retention time=1.381 min. Method C1

Chiral SFC: CO₂/MeOH containing 0.2% MA (65%:35%) over CHIRALPAK® IG column (4.6*100 mm 5 μm), retention time=1.71 min).

Compounds 2224, 2225, 2226, 2227, 2208, 2209, 2210, 2220, 2221, 2216, 2217, 2218, 2219, 2232, 2233, 2234, 2235, 2223, 2231, 2252, 2282, 2284, 2285, 2286, and 2287 were prepared in a similar manner to Compound 2222.

Diastereomers COMPOUND 2224 and COMPOUND 2227, and COMPOUND 2225 and COMPOUND 2226 were separated by chiral SFC eluting with 35% EtOH 1% MA over an EnantioPak® IC column to give COMPOUND 2224 (retention time=1.709 min) and COMPOUND 2227 (retention time=3.64 min), COMPOUND 2225 (retention time=2.090 min) and COMPOUND 2226 (retention time=2.844 min). Stereochemical assignment of (S) at the 1 position of the tetrahydroisoquinoline is based on enantiomerically pure starting materials; stereochemical assignment at the pyrrolidine chiral center is arbitrarily assigned based on chromatographic elution order.

COMPOUND 2224: LCMS: (M+H)⁺ 380.2; retention time=1.481 min. Method C

COMPOUND 2225: LCMS: (M+H)⁺ 380.1; retention time=1.483 min. Method C

COMPOUND 2226: LCMS: (M+H)⁺ 380.2; retention time=1.483 min. Method C

COMPOUND 2227: LCMS: (M+H)⁺ 380.1; retention time=1.484 min. Method C

COMPOUND 2208: LCMS: (M+H)⁺ 394.1; retention time=1.599 min. Method C

Diastereomers COMPOUND 2209 and COMPOUND 2210 were separated by chiral SFC eluting with CO₂/MeOH containing 0.2% methanolic ammonia over an EnantioPak® IG column (4.6*100 mm 5 μm) to give COMPOUND 2209 (retention time=1.986 min) and COMPOUND 2210 (retention time=2.918 min). Stereochemical assignment of (S) at the 1 position of the tetrahydroisoquinoline is based on enantiomerically pure starting materials; the stereochemical assignment at the morpholine chiral center is arbitrary and based on chromatographic elution order as compared to related analogues of known configuration.

COMPOUND 2209: LCMS: (M+H)⁺=370; retention time=1.555 min. Method D

COMPOUND 2210: LCMS: (M+H)⁺=370; retention time=1.553 min. Method D

Diastereomers COMPOUND 2220 and COMPOUND 2221 were separated by chiral HPLC eluting with n-Hexane 0.1% DEA):EtOH (0.1% DEA)=60:40 over an EnantioPak® AY column (4.6*250 mm 5 μm) to give COMPOUND 2220 (retention time=4.81 min), COMPOUND 2221 (retention time=7.29 min). Stereochemical assignment of (S) at the 1 position of the tetrahydroisoquinoline is based on enantiomerically pure starting materials; the stereochemical assignment at the piperidine chiral center is arbitrary based on chromatographic elution order as compared to related analogues of known configuration.

COMPOUND 2220: LCMS: (M+H)⁺=368; retention time=1.348 min. LCMS CP Method A

COMPOUND 2221: LCMS: (M+H)⁺=368; retention time=1.345 min. LCMS CP Method A

Diastereomers COMPOUND 2216 and COMPOUND 2217, and COMPOUND 2218 and COMPOUND 2219 were separated by chiral SFC eluting with 30% EtOH 1% MA over an EnantioPak® IE column to give COMPOUND 2216 (retention time=2.509 min) and COMPOUND 2217 (retention time=2.985 min), and COMPOUND 2218 (retention time=1.691 min) and COMPOUND 2219 (retention time=2.360 min). Stereochemistry is arbitrarily assigned based on chromatographic elution order as compared to diastereomers of related analogues of known configuration.

COMPOUND 2216: LCMS: (M+H)⁺ 354.1; retention time=1.645 min. Method C

COMPOUND 2217: LCMS: (M+H)⁺ 354.1; retention time=1.655 min. Method C

COMPOUND 2218: LCMS: (M+H)⁺ 354.1; retention time=1.697 min. Method C

COMPOUND 2219: LCMS: (M+H)⁺ 354.1; retention time=1.701 min. Method C

Diastereomers COMPOUND 2232 and COMPOUND 2233 and COMPOUND 2234 and COMPOUND 2235 were separated by chiral HPLC eluting with n-Hexane (0.1% DEA):EtOH (0.1% DEA)=70:30 over an EnantioPak® AY-H column (250*4.6 mm Sum) to give COMPOUND 2232 (retention time=5.972 min) and COMPOUND 2233 (retention time=7.089 min), and COMPOUND 2234 (retention time=5.768 min) and COMPOUND 2235 (retention time=7.068 min). Stereochemistry is arbitrarily assigned based on chromatographic elution order as compared to diastereomers of related analogues of known configuration.

COMPOUND 2232: LCMS: (M+H)⁺ 354.1; retention time=1.457 min. Method C

COMPOUND 2233: LCMS: (M+H)⁺ 354.1; retention time=1.454 min. Method C

COMPOUND 2234: LCMS: (M+H)⁺ 354.1; retention time=1.683 min. Method C

COMPOUND 2235: LCMS: (M+H)⁺ 354.1; retention time=1.680 min. Method C

COMPOUND 2223: LCMS: (M+H)⁺=340; retention time=1.521 min. Method C

COMPOUND 2231: LCMS: (M+H)⁺ 358.1; retention time=1.577 min. Method C

COMPOUND 2252: LCMS: (M+H)⁺ 380.1; retention time=1.474 min. Method C

COMPOUND 2282: LCMS: (M+H)⁺=354; retention time=1.364 min. Method E

Diastereomers COMPOUND 2284 and COMPOUND 2285 were separated by chiral SFC eluting with CO₂/EtOH containing 1% methanolic ammonia over a Daicel® AD column (20×250 mm, 10 μm) to give COMPOUND 2284 and COMPOUND 2285. Stereochemical assignment of (S) at the pyrrolidine chiral center is based on enantiomerically pure starting material; stereochemistry at the 1 position of the tetrahydroisoquinoline is arbitrarily assigned based on chromatographic elution order as compared to related analogues of known configuration.

COMPOUND 2284: LCMS: (M+H)⁺=340; retention time=1.373 min. Method B

COMPOUND 2285: LCMS: (M+H)⁺=340; Retention time=1.372 min. Method B

Diastereomers COMPOUND 2286 and COMPOUND 2287 were separated by chiral SFC eluting with CO₂/EtOH containing 1% methanolic ammonia over a Daicel® AD column (20×250 mm, 10 μm) to give COMPOUND 2286 and COMPOUND 2287.

Stereochemical assignment of (R) at the pyrrolidine chiral center is based on enantiomerically pure starting material; stereochemistry at the 1 position of the tetrahydroisoquinoline is arbitrarily assigned based on chromatographic elution order as compared to related analogues of known configuration.

COMPOUND 2286: LCMS: (M+H)⁺=340; retention time=1.371 min. Method B

COMPOUND 2287: LCMS: (M+H)⁺=340; retention time=1.371 min. Method B

Synthesis of COMPOUND 2239

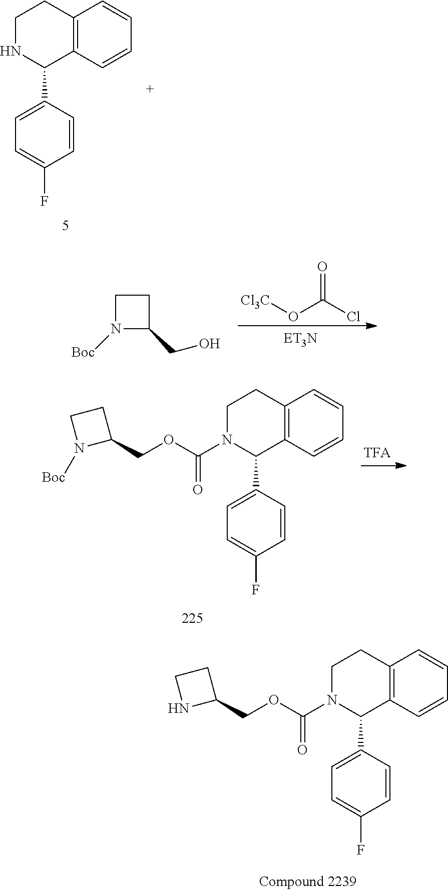

Step 1: To a solution of (S)-tert-butyl 2-(hydroxymethyl)azetidine-1-carboxylate (187 mg, 1.0 mmol) in MeCN (5 mL) was added trichloromethyl carbonochloridate (198 mg, 1.0 mmol). The reaction mixture was stirred at room temperature for 2 h and then concentrated under reduced pressure to give a white solid which was dissolved in DMF (5 mL). To the DMF solution were added (S)-1-(4-fluorophenyl)-1,2,3,4-tetrahydroisoquinoline (227 mg, 1.0 mmol) and TEA (303 mg, 3.0 mmol) and the reaction mixture was stirred at 90° C. overnight. The mixture was cooled to 25° C. and water (10 mL) was added. The mixture was extracted with ethyl acetate (3×20 mL) and the combined organic phase was washed with brine (3×15 mL), dried and concentrated in vacuo to give a crude product. This was purified by Prep-HPLC to give (S)-((S)-1-(tert-butoxycarbonyl)azetidin-2-yl)methyl 1-(4-fluorophenyl)-3,4-dihydroisoquinoline-2(1H)-carboxylate.

LCMS: (M−55)⁺=385; Retention time=1.850 min. LCMS CP Method C

Step 2: To a solution of (S)-((S)-1-(tert-butoxycarbonyl)azetidin-2-yl)methyl 1-(4-fluorophenyl)-3,4-dihydroisoquinoline-2(1H)-carboxylate (220 mg, 0.5 mmol) in DCM (2 mL) was added TFA (1 mL). The reaction mixture was stirred at room temperature for 2 h, concentrated and the residue purified by Prep-HPLC to give (S)-(S)-azetidin-2-ylmethyl 1-(4-fluorophenyl)-3,4-dihydroisoquinoline-2(1H)-carboxylate.

LCMS: (M+H)⁺=341; Retention time=1.401 min. LCMS CP Method A

COMPOUND 2239: LCMS: (M+H)⁺=341; (214 nm); retention time=1.400 min. LCMS CP Method A

Chiral SFC: CO₂/MeOH containing 0.2% MA (65%:35%) over CHIRALPAK® IG column (4.6*250 mm 5 μm), retention time=1.164 min).

Compound 2240 was prepared following a similar synthesis as for Compound 2239. COMPOUND 2240: LCMS: (M+H)⁺=341; retention time=1.386 min. Method A

Synthesis of COMPOUND 2241 and COMPOUND 2242

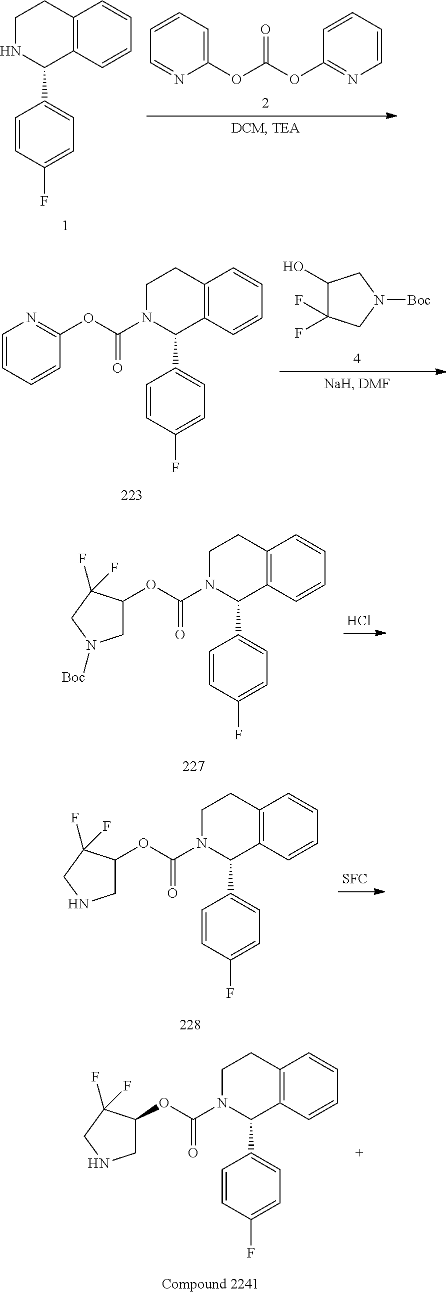

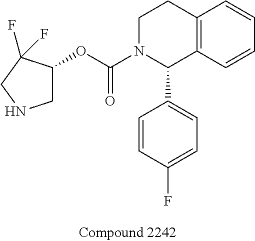 [SMILES: O=C(O[C@@H]1CNCC1(F)F)N1CCc2ccccc2[C@@H]1c1ccc(F)cc1]

Step 1: To a solution of (S)-1-(4-fluorophenyl)-1,2,3,4-tetrahydroisoquinoline (0.5 g, 2.2 mmol) and TEA (0.92 mL, 6.6 mmol) in DCM (15 mL) was added di(pyridin-2-yl) carbonate (0.95 g, 4.4 mmol) and the reaction mixture was stirred at room temperature for 16 h. Then the mixture was diluted with DCM (60 mL) and washed with water (20 mL×2) and brine (20 mL×2). The organic layer was dried over Na₂SO₄, filtered and concentrated to give crude pyridin-2-yl (S)-1-(4-fluorophenyl)-3,4-dihydroisoquinoline-2(1H)-carboxylate which was used directly in the next step reaction without further purification.

LCMS: (M+1)⁺=349; Retention time=1.822 min. LCMS CP Method B

Step 2: To a solution of tert-butyl 3,3-difluoro-4-hydroxypyrrolidine-1-carboxylate (0.3 g, 1.34 mmol) in dry DMF (8 mL) was added NaH (108 mg, 2.96 mmol, 60% in mineral oil) at 0° C. The reaction mixture was stirred at room temperature for 1 h before a solution of pyridin-2-yl (S)-1-(4-fluorophenyl)-3,4-dihydroisoquinoline-2(1H)-carboxylate (0.515 g, 1.48 mmol) in dry DMF (1 mL) was added. The reaction mixture was heated to 70° C. for 1 h before being cooled to ambient temperature and filtered. The filtrate was concentrated and the residue purified by Prep-HPLC to give 1-(tert-butoxycarbonyl)-4,4-difluoropyrrolidin-3-yl (1S)-1-(4-fluorophenyl)-3,4-dihydroisoquinoline-2(1H)-carboxylate.

LCMS: (M−55)⁺=422; Retention time=2.007 min. LCMS CP Method B

Step 3: To a solution of 1-(tert-butoxycarbonyl)-4,4-difluoropyrrolidin-3-yl (1S)-1-(4-fluorophenyl)-3,4-dihydroisoquinoline-2(1H)-carboxylate (450 mg, 0.9 mmol) in DCM (3 mL) was added HCl (1 mL, 3.6 mmol, 4 M dioxane) at room temperature. The reaction mixture was stirred at ambient temperature for 16 h, concentrated under reduced pressure and the residue dissolved in water (20 mL). The pH of the mixture was adjusted to 8 by the addition of NaHCO₃ and extracted with DCM (3×30 mL). The organic phase was washed with water, brine, dried over Na₂SO₄, and filtered. Concentration of the filtrate provided 4,4-difluoropyrrolin-3-yl-(1 S)-1-(4-fluorophenyl-3,4-dihydroisoquinoline-2(1H)-carboxylate.

LCMS: (M+1)⁺=377; Retention time=1.520 min. LCMS CP Method B

The diastereomers were separated by chiral SFC eluting with CO₂/EtOH containing 0.2% methanolic ammonia over a Daicel® AD column (20×250 mm, 10 μm) to give COMPOUND 2241 and COMPOUND 2242. Stereochemical assignment of (S) at the 1 position of the tetrahydroisoquinoline is based on enantiomerically pure starting materials; the stereochemistry at the pyrrolidine chiral center is arbitrarily assigned based on chromatographic elution order as compared to related analogues of known configuration.

COMPOUND 2241: LCMS: (M+H)⁺=377; Retention time=1.857 min. LCMS CP Method C

Chiral SFC: CO₂/MeOH containing 0.2% ammonia (85%:15%) over CHIRALPAK® IG column (4.6×100 mm, 5 μm), retention time=2.143 min).

COMPOUND 2242: LCMS: (M+H)⁺=377; Retention time=1.866 min. LCMS CP Method C

Chiral SFC: CO₂/MeOH containing 0.2% ammonia (85%:15%) over CHIRALPAK® IG column (4.6×100 mm, 5 μm), retention time=2.290 min).

Compounds 2230, 2255, and 2256 were prepared following a similar synthesis as for Compounds 2241 and 2242.

COMPOUND 2230: LCMS: (M+H)⁺ 355.2; purity=98.69% (214 nm); retention time=1.488 min. Method C

Diastereomers COMPOUND 2255 and COMPOUND 2256 were separated by chiral HPLC eluting with n-Hexane (0.1% DEA):EtOH (0.1% DEA)=70:30 over an EnantioPak® IE column (4.6*250 mm 5 μm) to give COMPOUND 2255 (retention time=10.420 min) and COMPOUND 2256 (retention time=9.347 min). Stereochemical assignment of (S) at the 1 position of the tetrahydroisoquinoline is based on enantiomerically pure starting material; the stereochemistry at the pyrrolidine chiral center is arbitrarily assigned based on chromatographic elution order as compared to related analogues of known configuration.

COMPOUND 2255: LCMS: (M+H)⁺ 373.1; retention time=1.460 min. Method C

COMPOUND 2256: LCMS: (M+H)⁺ 373.1; retention time=1.468 min. Method C

Synthesis of COMPOUND 2243 and COMPOUND 2244

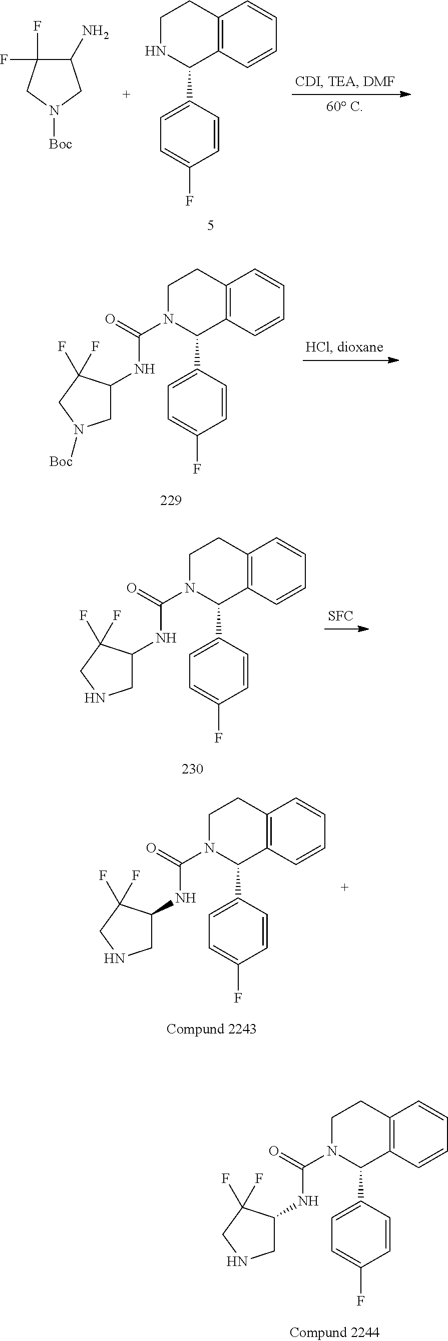

Step 1: To a solution of tert-butyl 4-amino-3,3-difluoropyrrolidine-1-carboxylate (220 mg, 1 mmol) and TEA (0.18 mL, 1.3 mmol) in DMF (5 mL) were added (S)-1-(4-fluorophenyl)-1,2,3,4-tetrahydroisoquinoline (150 mg, 0.66 mmol) and CDI (214 mg, 1.3 mmol) and the resulting reaction mixture was stirred at 60° C. for 2 h. The mixture was diluted with ethyl acetate (60 mL) and water (30 mL). The aqueous layer was extracted with ethyl acetate (3×40 mL). The combined organic layers were washed with brine (30 mL), dried over Na₂SO₄, filtered and concentrated. The residue was purified by column chromatography to give tert-butyl 3,3-difluoro-4-((S)-1-(4-fluorophenyl)-1,2,3,4-tetrahydroisoquinoline-2-carboxamido)pyrrolidine-1-carboxylate.

LCMS: (M−55)⁺=420; Retention time=1.870 min. LCMS CP Method F

Step 2: To a solution of tert-butyl 3,3-difluoro-4-((S)-1-(4-fluorophenyl)-1,2,3,4-tetrahydroisoquinoline-2-carboxamido)pyrrolidine-1-carboxylate (200 mg, 0.4 mmol) in dioxane (2 mL) was added HCl in dioxane (4N, 2 mL) at 0° C. The reaction mixture was stirred at room temperature for 2 h and then concentrated under reduced pressure. The residue was purified by Prep-HPLC to give of (1 S)—N-(4,4-difluoropyrrolidin-3-yl)-1-(4-fluorophenyl)-3,4-dihydroisoquinoline-2(1H)-carboxamide.

LCMS: (M+H)⁺=376; Retention time=1.490 min. LCMS CP Method F

The diastereomers were separated by chiral SFC eluting with CO₂/EtOH containing 0.2% Methanol Ammonia over an EnantioPak® AD column (20*250 mm 10 μm) to give COMPOUND 2243 (retention time=3.101 min) and COMPOUND 2244 (retention time=1.651 min). Stereochemical assignment of (S) at the 1 position of the tetrahydroisoquinoline is based on enantiomerically pure starting materials; the stereochemistry at the pyrrolidine chiral center is arbitrarily assigned based on chromatographic elution order as compared to related analogues of known configuration.

COMPOUND 2243: LCMS: (M+H)⁺=376; (214 nm); retention time=1.493 min. LCMS CP Method F

Chiral SFC: CO₂/EtOH containing 1% MA (55%:45%) over a CHIRALPAK® IG column (4.6*100 mm 5 μm), retention time=1.444 min.

COMPOUND 2244: LCMS: (M+H)⁺=376; (214 nm); retention time=1.502 min. LCMS CP Method F

Chiral SFC: CO₂/EtOH containing 1% MA (55%:45%) over a CHIRALPAK® IG column (4.6*100 mm 5 μm), retention time=1.762 min.

Compounds 2280, 2281, 2273, and 2268 were prepared following a similar synthesis as for Compounds 2243 and 2244.

Diastereomers COMPOUND 2280 and COMPOUND 2281 were separated by chiral SFC eluting with CO₂/MeOH containing 0.2% methanol ammonia over an EnantioPak© AD column (4.6*250 mm 5 μm) to give COMPOUND 2280 (retention time=0.911 min), COMPOUND 2281 (retention time=2.225 min). Stereochemical assignment of (S) at the 1 position of the tetrahydroisoquinoline is based on enantiomerically pure starting material; the stereochemistry at the pyrrolidine chiral center is arbitrarily assigned based on chromatographic elution order as compared to related analogues of known configuration.

COMPOUND 2280: LCMS: (M+H)⁺=366; retention time=1.511 min. Method F

COMPOUND 2281: LCMS: (M+H)⁺=366; retention time=1.529 min. Method F

COMPOUND 2273: LCMS: (M+H)⁺=368; retention time=1.439 min. Method F

COMPOUND 2268: LCMS: (M+H)⁺=368; retention time=1.417 min. LCMS CP Method F

Synthesis of COMPOUND 2253 and COMPOUND 2254

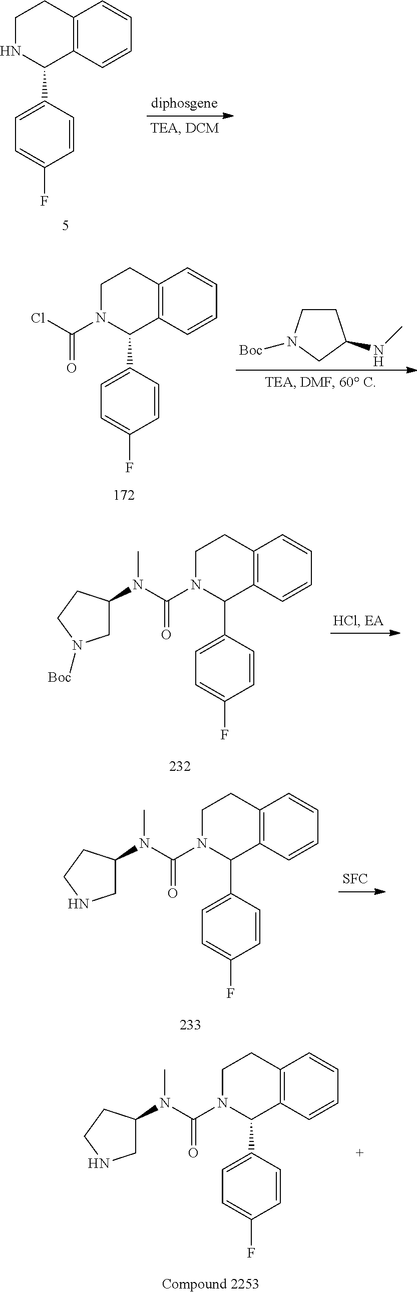

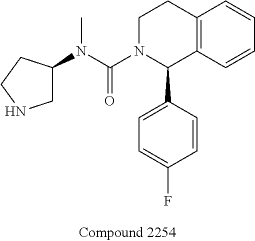 [SMILES: CN(C(=O)N1CCc2ccccc2[C@H]1c1ccc(F)cc1)[C@@H]1CCNC1]

Step 1: To a solution of (S)-1-(4-fluorophenyl)-1,2,3,4-tetrahydroisoquinoline (200 mg, 0.9 mmol) and TEA (0.24 mL, 1.7 mmol) in DCM (4 mL) at 0° C. was added diphosgene (0.14 mL, 1.1 mmol). The resulting reaction mixture was stirred at 0° C. for 2 h and then concentrated under reduced pressure to give crude (S)-1-(4-fluorophenyl)-3,4-dihydroisoquinoline-2(1H-carbonyl chloride which was used directly in the next step reaction without further purification.

Step 2: To a solution of (R)-tert-butyl 3-(methylamino)pyrrolidine-1-carboxylate (150 mg, 0.7 mmol) and TEA (0.5 mL, 3.7 mmol) in DMF (2 mL) was added a solution of (S)-1-(4-fluorophenyl)-3,4-dihydroisoquinoline-2(1H-carbonyl chloride (240 mg, 0.8 mmol) in DMF (2 mL). The reaction mixture was stirred at 60° C. for 2 h and then diluted with ethyl acetate (60 mL) and water (20 mL). The aqueous layer was extracted with ethyl acetate (3×30 mL) and the combined organic layers were washed with brine (30 mL), dried over Na₂SO₄, filtered and concentrated. The residue was purified by column chromatography to give (R)-tert-butyl 3-((S)-1-(4-fluorophenyl)-N-methyl-1,2,3,4-tetrahydroisoquinoline-2-carboxamido)pyrrolidine-1-carboxylate.

LCMS: (M−55)⁺=398; Retention time=2.207 min. LCMS CP Method E

Step 3: To a solution of tert-butyl 3-((S)-1-(4-fluorophenyl)-N-methyl-1,2,3,4-tetrahydroisoquinoline-2-carboxamido)piperidine-1-carboxylate (200 mg, 0.44 mmol) in ethyl acetate (2 mL) was added HCl in ethyl acetate (3N, 4 mL) at 0° C. The reaction mixture was stirred at room temperature for 2 h and then concentrated under reduced pressure. The residue was purified by Prep-HPLC to give 1-(4-fluorophenyl)-N-methyl-((R)-pyrrolin-3-yl)-3,4-dihydroisoquinoline-2(1H)-carboxamide

LCMS: (M+H)⁺=354; Retention time=1.500 min. LCMS CP Method F

The diastereomers were separated by chiral SFC eluting with CO₂/MeOH containing 0.2% methanol ammonia over an EnantioPak® IG column (4.6*250 mm 5 μm) to give COMPOUND 2253 (retention time=1.51 min), COMPOUND 2254 (retention time=2.59 min). Stereochemical assignment of (R) at the pyrrolidine chiral center is absolute based on starting materials; stereochemical assignment at 1 position of the tetrahydroisoquinoline is arbitrarily assigned based on chromatographic elution order as compared to diastereomers of related analogues of known configuration.

COMPOUND 2253: LCMS: (M+H)⁺=354; (214 nm); retention time=1.500 min. LCMS CP Method F

Chiral SFC: CO₂/MeOH containing 0.2% ammonia (60%:40%) over a CHIRALPAK® IG column (4.6*100 mm 5 μm), retention time=1.510 min.

COMPOUND 2254: LCMS: (M+H)⁺=354; (214 nm); retention time=1.499 min. LCMS CP Method F

Chiral SFC: CO₂/MeOH containing 0.2% ammonia (60%:40%) over a CHIRALPAK® IG column (4.6*100 mm 5 μm), retention time=2.590 min.

were prepared following a similar synthesis as for Compound 2253.

Diastereomers COMPOUND 2248 and COMPOUND 2249 were separated by chiral SFC eluting with CO₂/MeOH containing 0.2% methanol ammonia over an EnantioPak® IG column (4.6*250 mm 5 μm) to give COMPOUND 2248 (retention time=1.64 min), COMPOUND 2249 (retention time=2.1 min). Stereochemical assignment of (S) at the pyrrolidine chiral center is absolute based on starting materials; stereochemistry at the 1 position of the tetrahydroisoquinoline is arbitrarily assigned based on chromatographic elution order compared to diastereomers of related analogues of known configuration.

COMPOUND 2248: LCMS: (M+H)⁺=354; retention time=1.503 min. Method F

COMPOUND 2249: LCMS: (M+H)⁺=354; retention time=1.498 min. Method F

Synthesis of Compounds COMPOUND 2274 and COMPOUND 2267

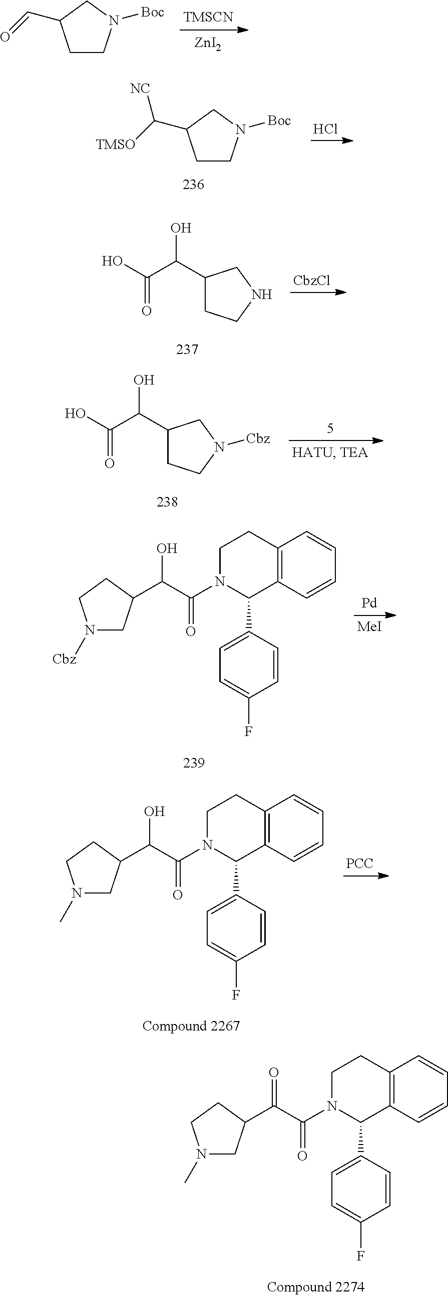

Step 1: To a solution of tert-butyl 3-formylpyrrolidine-1-carboxylate (3.98 g, 20.0 mmol) in anhydrous DCM (60 mL) were added TMSCN (2.38 g, 24.0 mmol) and ZnI₂ (319 mg, 1.0 mmol). The reaction mixture was stirred at room temperature overnight and then filtered. The filtrate was concentrated to give crude product tert-butyl 3-(cyano(trimethylsilyloxy)methyl)pyrrolidine-1-carboxylate which was used directly in the next step without further purification.

LCMS: (M+H)⁺=299; Retention time=1.551 min. LCMS CP Method E

Step 2: A suspension of tert-butyl 3-(cyano(trimethylsilyloxy)methyl)pyrrolidine-1-carboxylate (5.96 g, 20 mmol) in concentrated HCl (20 mL) was heated at reflux overnight. The mixture was concentrated under reduced pressure to give crude 2-hydroxy-2-(pyrrolidin-3-yl)acetic acid which was used directly in the next step.

LCMS: (M+H)⁺=160; Retention time=0.392 min. LCMS CP Method E

Step 3: To a solution of 2-hydroxy-2-(pyrrolidin-3-yl)acetic acid (1.45 g, 10.0 mmol) in a mixture of THF and H₂O (45 mL, 2:1) were added Na₂CO₃ (2.12 g, 20.0 mmol) and benzyl carbonochloridate (2.55 g, 15.0 mmol). The resulting reaction mixture was stirred at room temperature overnight and then the mixture extracted with DCM (3×30 ml). The combined organic layers were dried over Na₂SO₄, filtered and concentrated to give a residue which was purified by Prep-HPLC affording 2-(1-(benzyloxycarbonyl)pyrrolidin-3-yl)-2-hydroxyacetic acid.

LCMS: (M+Na)⁺=302; Retention time=1.390 min. LCMS CP Method E

Step 4: To a solution of 2-(1-(benzyloxycarbonyl)pyrrolidin-3-yl)-2-hydroxyacetic acid (831 mg, 3.0 mmol) in DMF (10 mL) were added (S)-1-(4-fluorophenyl)-1,2,3,4-tetrahydroisoquinoline (749 mg, 3.3 mmol), HATU (1.254 g, 3.3 mmol) and Et₃N (606 mg, 6.0 mmol) at 0° C. The resulting reaction mixture was stirred at room temperature for 2 h, diluted with EA (10 mL) and washed with saturated NH₄Cl (2×10 mL) followed by brine (2×10 mL). The organic phase was dried over Na₂SO₄, filtered and concentrated to give a residue which was purified by Prep-HPLC yielding the product.

LCMS: (M+H)⁺=489; Retention time=2.093 min. LCMS CP Method E

Step 5: To a solution of benzyl 3-(2-((S)-1-(4-fluorophenyl)-3,4-dihydroisoquinolin-2(1H)-yl)-1-hydroxy-2-oxoethyl)pyrrolidine-1-carboxylate (976 mg, 2.0 mmol) in MeOH (10 mL) was added Pd/C (100.0 mg, 10 wt %) and the reaction mixture was stirred at room temperature under a hydrogen atmosphere overnight. The reaction mixture was filtered and to the filtrate was added Mel (284 mg, 2.0 mmol). The solution was stirred at room temperature for 3 h and then concentrated. The residue was purified by Prep-HPLC to give 1-((S)-1-(4-fluorophenyl)-3,4-dihydroisoquinolin-2(1H)-yl)-2-hydroxy-2-(pyrrolidin-3-yl)ethanone.

LCMS: (M+H)⁺=355; Retention time=1.433 min. LCMS CP Method B

COMPOUND 2267: LCMS: (M+H)⁺=369; Retention time=1.427 min. LCMS CP Method C2

Step 6: To a solution of 1-((S)-1-(4-fluorophenyl)-3,4-dihydroisoquinolin-2(1H)-yl)-2-hydroxy-2-(1-methylpyrrolidin-3-yl)ethanone (73 mg, 0.2 mmol) in DCM (5 ml) was added PCC (130 mg, 0.6 mmol). The reaction mixture was stirred at room temperature overnight before water was added and the mixture extracted with DCM. The combined organic layers were dried over Na₂SO₄, filtered, and concentrated to give a residue which was purified by Prep-HPLC to give 1-((S)-1-(4-fluorophenyl)-3,4-dihydroisoquinolin-2(1H)-yl)-2-(1-methylpyrrolidin-3-yl)ethane-1,2-dione.

LCMS: (M+H)⁺=367; Retention time=1.901 min. LCMS CP Method C

COMPOUND 2274: LCMS: (M+H)⁺=367; Retention time=1.902 min. LCMS CP Method C

Chiral SFC: CO₂/MeOH containing 0.2% ammonia (65%:35%) over CHIRALPAK® IG column (4.6×100 mm, 5 μm), retention time=2.264 min).

Synthesis of COMPOUND 2309 and COMPOUND 2310

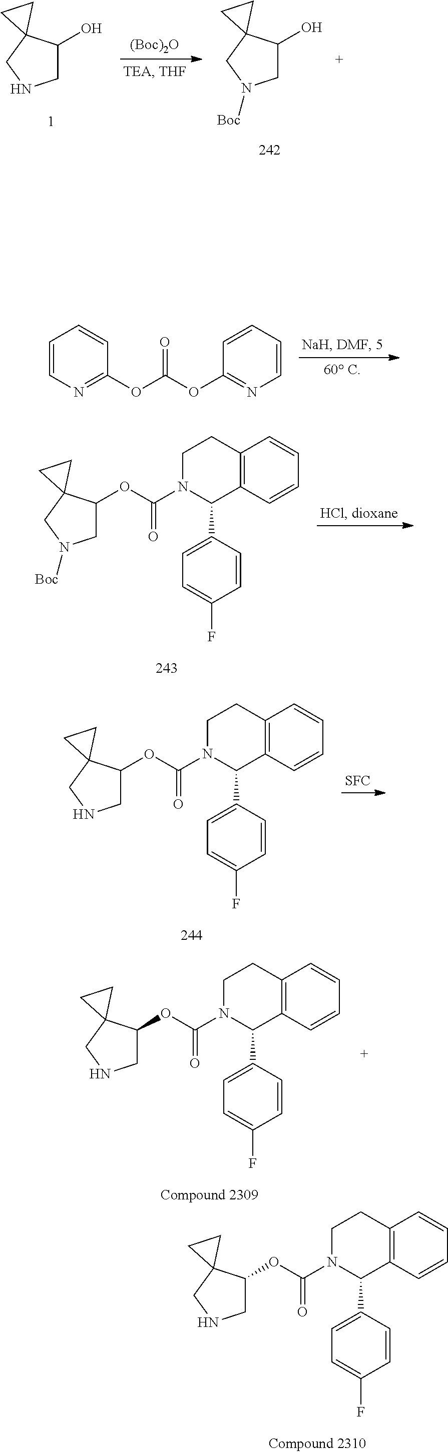

Step 1: To a solution of 5-azaspiro[2.4]heptan-7-ol hydrochloride (150 mg, 1.0 mmol) in THF (4 mL) were added TEA (0.28 mL, 2.0 mmol) and (Boc)₂O (229 mg, 1.05 mmol). The reaction mixture was stirred at room temperature for 2 h and then the mixture concentrated under reduced pressure. The residue was purified by column chromatography on silica gel eluting with 5% MeOH/DCM to give N-Boc protected 5-azaspiro[2.4]heptan-7-ol.

LCMS: (M−55)⁺=158; Retention time=1.273 min. LCMS CP Method C

Step 2: To a solution of N-Boc protected 5-azaspiro[2.4]heptan-7-ol (150 mg, 0.7 mmol), (S)-1-(4-Fluorophenyl)-1,2,3,4-Tetrahydroisoquinoline (106 mg, 0.5 mmol) and dipyridin-2-yl carbonate (152 mg, 0.7 mmol) in DMF (5 mL) was added NaH (75 mg, 1.9 mmol) at 60° C. After stirring the mixture at this temperature for 3 h, it was diluted with ethyl acetate (60 mL) and water (30 mL). The aqueous layer was extracted with ethyl acetate (3×50 mL) and the combined organic layers were washed with brine (40 mL), dried over Na₂SO₄, filtered and concentrated. The residue was purified by column chromatography to give 5-(t-butoxycarbonyl)-5-azaspiro[2.4]hepan-7-yl-(1S)-1-(4-fluorophenyl)-3,4-dihydroisoquinoline-2(1H)-carboxylate.

LCMS: (M−55)⁺=411; Retention time=2.041 min. LCMS CP Method F

Step 3: To a solution of 5-(tert-butoxycarbonyl)-5-azaspiro[2.4]heptan-7-yl (1 S)-1-(4-fluorophenyl)-3,4-dihydroisoquinoline-2(1H-carb-oxylate (80 mg, 0.17 mmol) in dioxane (2 mL) was added HCl in dioxane (4N, 2 mL) at 0° C. The reaction mixture was stirred at room temperature for 2 hand then concentrated under reduced pressure. The residue was purified by Prep-HPLC to give 5-azaspiro[2.4]heptan-7-yl-(1 S)-1-(4-fluorophenyl)-3,4-dihydroisoquinoline-2(1H)-carboxylate.

LCMS: (M+H)⁺=367; Retention time=1.522 min. LCMS CP Method F

The diastereomers were separated by chiral HPLC eluting with n-Hexane (0.1% DEA)/EtOH (0.1% DEA) over an EnantioPak© IG column (4.6*250 mm 5 μm) to give COMPOUND 2309 (retention time=19.295 min), COMPOUND 2310 (retention time=10.459 min). Stereochemical assignment of (S) at the 1 position of the tetrahydroisoquinoline is based on enantiomerically pure starting material; the stereochemistry at the pyrrolidine chiral center is arbitrarily assigned based on chromatographic elution order as compared to related analogues of known configuration.

COMPOUND 2309: LCMS: (M+H)⁺=367; (214 nm); retention time=1.521 min. LCMS CP Method F

Chiral HPLC: n-Hexane (0.1% DEA):EtOH (0.1% DEA)=70:30 over CHIRALPAK® IG column (4.6*100 mm 5 μm), retention time=19.295 min.

COMPOUND 2310: LCMS: (M+H)⁺=367; (214 nm); retention time=1.519 min. LCMS CP Method F

Chiral HPLC: n-Hexane (0.1% DEA):EtOH (0.1% DEA)=70:30 over CHIRALPAK® IG column (4.6*100 mm 5 μm), retention time=10.459 min.

Synthesis of COMPOUND 2291 and COMPOUND 2292

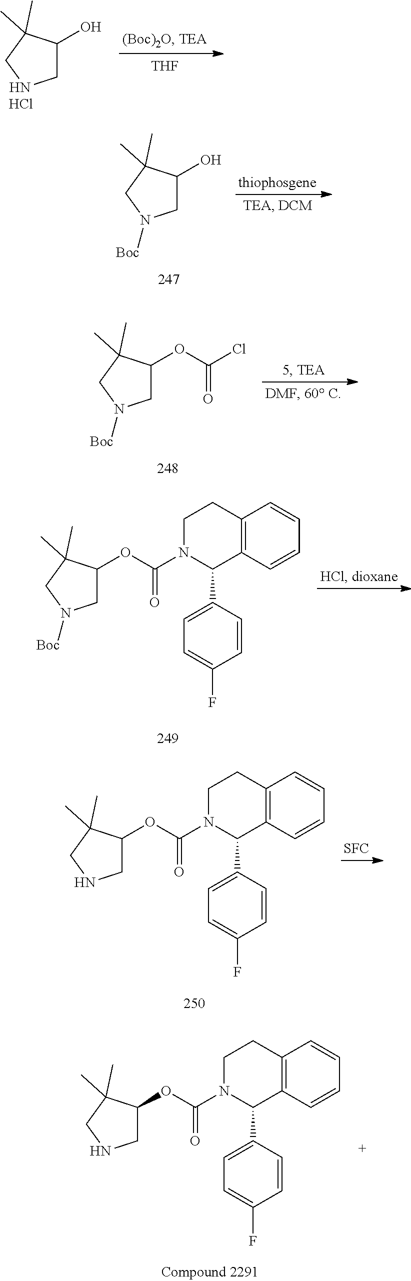

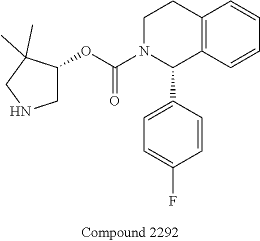 [SMILES: CC1(C)CNC[C@H]1OC(=O)N1CCc2ccccc2[C@@H]1c1ccc(F)cc1]

Step 1: To a solution of 4,4-dimethylpyrrolidin-3-ol hydrochloride (300 mg, 2.0 mmol) in THF (6 mL) were added TEA (0.55 mL, 4.0 mmol) and (Boc)₂O (453 mg, 2.1 mmol). The reaction mixture was stirred at room temperature overnight and then concentrated under reduced pressure. The residue was purified by silica gel chromatography (DCM:MeOH, 20:1) to give tert-butyl 4-hydroxy-3,3-dimethylpyrrolidine-1-carboxylate.

LCMS: (M−55)⁺=160; Retention time=1.522 min. LCMS CP Method F

Step 2: To a solution of tert-butyl 4-hydroxy-3,3-dimethylpyrrolidine-1-carboxylate 2 (200 mg, 0.9 mmol) in DCM (5 mL) were added TEA (0.26 mL, 1.9 mmol) and diphosgene (0.15 mL, 1.2 mmol) at 0° C. The resulting reaction mixture was stirred at the same temperature for 2 h and then concentrated to give crude tert-butyl 4-(chlorocarbonyloxy)-3,3-dimethylpyrrolidine-1-carboxylate which was used directly in the next step reaction without further purification.

Step 3: To a solution of (S)-1-(4-Fluorophenyl)-1,2,3,4-tetrahydroisoquinoline (120 mg, 0.5 mmol) in DMF (3 mL) were added TEA (0.15 mL, 1.1 mmol) and a solution of tert-butyl 4-(chlorocarbonyloxy)-3,3-dimethylpyrrolidine-1-carboxylate (220 mg, 0.8 mmol) in DMF (2 mL). The reaction mixture was stirred at 60° C. for 2 h and then diluted with ethyl acetate (60 mL) and water (20 mL). The aqueous layer was extracted with ethyl acetate (3×30 mL) and the combined organic layers were washed with brine (30 mL), dried over Na₂SO₄, filtered and concentrated. The residue which was purified by column chromatography to give (1S)-1-(t-butoxycarbonyl)-4,4-dimethylpyrrolindin-3-yl-(4-fluorophenyl)-3,4-dihydroisoquinoline-2(1H)-carboxylate.

LCMS: (M−55)⁺=413; Retention time=2.066 min. LCMS CP Method F

Step 4: To a solution of (1 S)-1-(tert-butoxycarbonyl)-4,4-dimethylpyrrolidin-3-yl 1-(4-fluorophenyl)-3,4-dihydroisoquinoline-2(1H-carboxylate (150 mg, 0.3 mmol) in dioxane (2 mL) was added HCl in dioxane (4N, 2 mL) at 0° C. The reaction mixture was stirred at room temperature for 2 h and then concentrated under reduced pressure. The residue was purified by Prep-HPLC to give (1 S)-1-(4-fluorophenyl)-N-methyl-N-(piperidin-3-yl)-3,4-dihydroisoquinoline-2(1H)-carboxamide.

LCMS: (M+H)⁺=369; Retention time=1.552 min. LCMS CP Method F

The diastereomers were separated by chiral SFC eluting with CO₂/MeOH containing 0.2% methanol ammonia over an EnantioPak® IG column (4.6*250 mm 5 μm) to give COMPOUND 2291 (retention time=1.251 min), COMPOUND 2292 (retention time=0.832 min). Stereochemical assignment of (S) at the 1 position of the tetrahydroisoquinoline is based on enantiomerically pure starting materials; the stereochemistry at the pyrrolidine chiral center is arbitrarily assigned based on chromatographic elution order as compared to related analogues of known configuration.

COMPOUND 2291: LCMS: (M+H)⁺=369; (214 nm); retention time=1.547 min. LCMS CP Method F

Chiral SFC: CO₂/IPA containing 1% ammonia (60%:40%) over CHIRALPAK® IG column (4.6*100 mm 5 μm), retention time=1.251 min.

COMPOUND 2292: LCMS: (M+H)⁺=369; (214 nm); retention time=1.577 min. LCMS CP Method F

Chiral SFC: CO₂/IPA containing 1% ammonia (60%:40%) over CHIRALPAK® IG column (4.6*100 mm 5 μm), retention time=0.832 min.

Synthesis of COMPOUND 2283

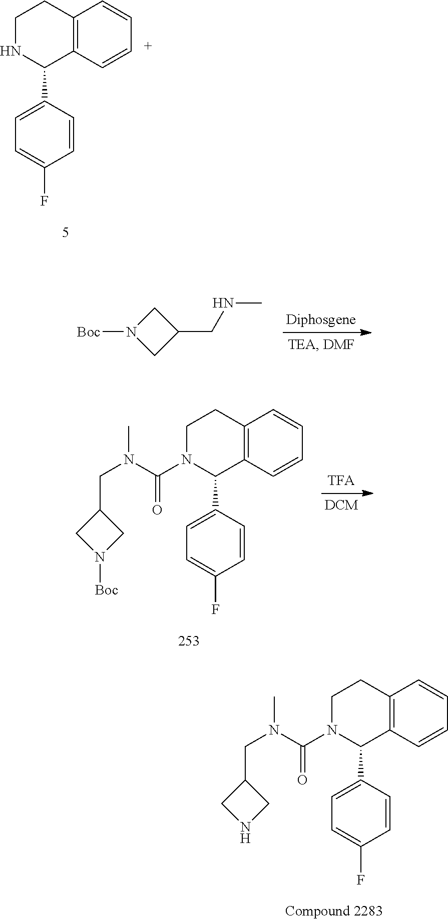 [SMILES: Fc1ccc([C@@H]2NCCc3ccccc32)cc1 ; CNCC1CN(C(=O)OC(C)(C)C)C1 ; CN(CC1CN(C(=O)OC(C)(C)C)C1)C(=O)N1CCc2ccccc2[C@@H]1c1ccc(F)cc1 ; CN(CC1CNC1)C(=O)N1CCc2ccccc2[C@@H]1c1ccc(F)cc1]

Step 1: To a solution of (S)-1-(4-fluorophenyl)-1,2,3,4-tetrahydroisoquinoline (400 mg, 1.76 mmol) in dry CH₃CN (10 mL) was added diphosgene (0.32 mL, 2.64 mmol mmol) at 0° C. The reaction mixture was stirred at room temperature for 2 h then concentrated and the residue was dissolved in dry DMF (10 mL). tert-butyl 3-((methylamino)methyl)azetidine-1-carboxylate (0.35 g, 1.76 mmol) and TEA (0.86 mL, 6.16 mmol) were added and the reaction mixture was stirred at room temperature for 16 h. To the mixture was added water (20 mL) and the mixture was extracted with EA (3×20 mL). The combined organic layers were washed with brine and dried over Na₂SO₄. Filtered and concentrated to give a residue which was purified by Prep-HPLC to give tert-butyl (S)-3-((1-(4-fluorophenyl)-N-methyl-1,2,3,4-tetrahydroisoquinoline-2-carboxamido)methyl)azetidine-1-carboxylate.

LCMS: (M+1)⁺=454; Retention time=1.899 min. LCMS CP Method B

Step 2: To a solution of tert-butyl (S)-3-((1-(4-fluorophenyl)-N-methyl-1,2,3,4-tetrahydroisoquinoline-2-carboxamido)methyl)azetidine-1-carboxylate (446 mg, 0.98 mmol) in DCM (5 mL) was added TFA (1 mL) at 0° C. The reaction mixture was stirred at room temperature for 3 h and then concentrated. The residue was dissolved in water and the pH was adjusted to 10 by the addition of NaOH. The mixture was extracted with DCM (30 mL×3), the combined organic phase concentrated and the crude residue purified by Prep-HPLC to give (S)—N-(azetidin-3-ylmethyl)-1-(4-fluorophenyl)-N-methyl-3,4-dihydroisoquinoline-2(1H)-carboxamide.

COMPOUND 2283: LCMS: (M+H)⁺=354; (214 nm); Retention time=1.552 min. LCMS CP Method C

Chiral SFC: CO₂/MeOH containing 0.2% ammonia (60%:40%) over CHIRALPAK® IG column (4.6×100 mm, 5 μm), retention time=0.490 min).

Synthesis of COMPOUND 2311

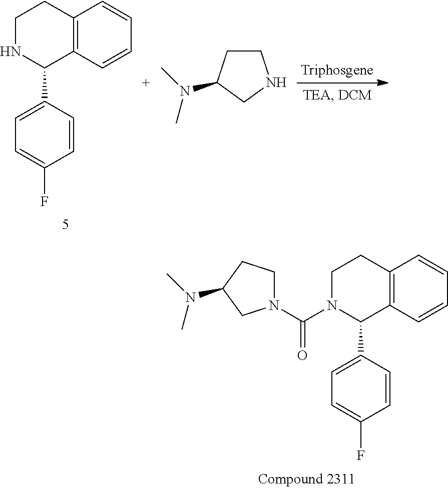

Step 1: To a solution of (S)-1-(4-fluorophenyl)-1,2,3,4-tetrahydroisoquinoline (136 mg, 0.6 mmol) and Et₃N (120 mg, 1.2 mmol) in DCM (2 mL) under nitrogen atmosphere was slowly added triphosgene (59 mg, 0.2 mmol). The mixture was stirred at room temperature for one hour and then concentrated to give crude (S)-1-(4-fluorophenyl)-3,4-dihydroisoquinoline-2(1H)-carbonyl chloride. This intermediate was dissolved in DCM (1 mL) and added to a solution of (R)—N,N-dimethylpyrrolidin-3-amine (68 mg, 0.6 mmol) and Et₃N (62 mg, 0.6 mmol) in DCM (2 mL). The mixture was stirred at room temperature for 2 h. Water was added to quench the reaction and the mixture was extracted with DCM. The combined organic layers were dried over Na₂SO₄, filtered, and concentrated to give a residue which was purified by Prep-HPLC to give ((R)-3-(dimethylamino)pyrrolidin-1-yl)((S)-1-(4-fluorophenyl)-3,4-dihydroisoquinolin-2(1H)-yl)methanone.

LCMS: (M+H)⁺=368; Retention time=1.821 min. LCMS CP Method C

COMPOUND 2311: LCMS: (M+H)⁺=368; purity=100% (214 nm); retention time=1.821 min. LCMS CP Method C

Chiral SFC: CO₂/MeOH containing 0.2% ammonia (75%:25%) over CHIRALPAK® IG column (4.6*250 mm 5 μm), retention time=2.690 min).

Compound 2312 was prepared following a similar synthesis as for Compound 2311.

COMPOUND 2312: LCMS: (M+H)⁺=368; retention time=1.806 min. Method C

Synthesis of COMPOUND 2315, COMPOUND 2316, COMPOUND 2317, and COMPOUND 2318

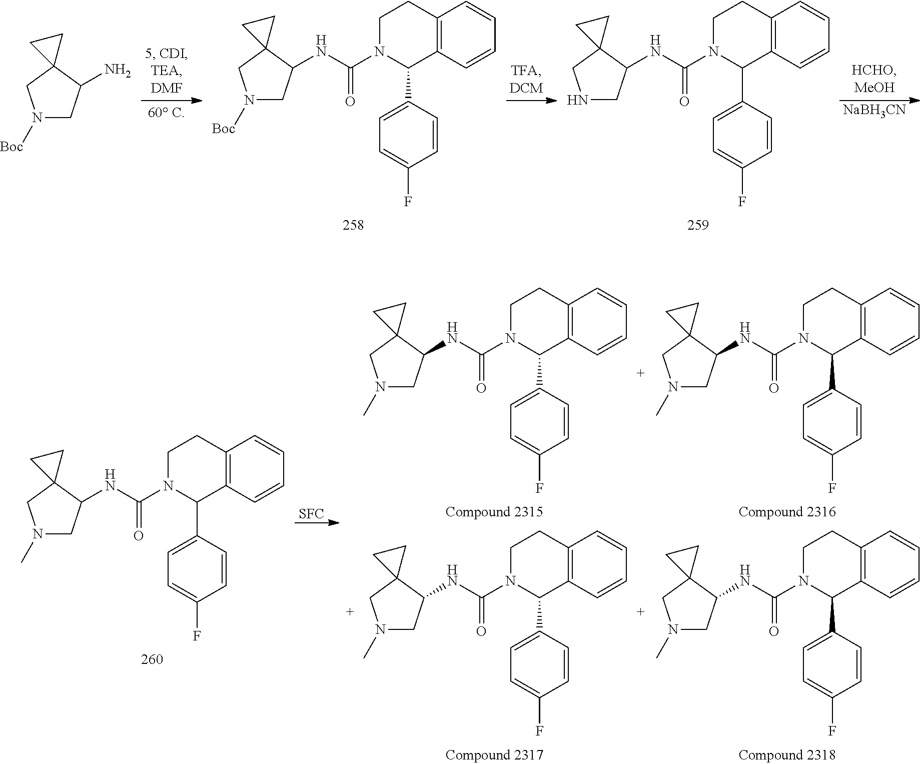

Step 1: To a solution of tert-butyl 7-amino-5-azaspiro[2.4]heptane-5-carboxylate (180 mg, 1.3 mmol) in DMF (5 mL) were added TEA (0.18 mL, 1.3 mmol), (S)-1-(4-fluorophenyl)-1,2,3,4-tetrahydroisoquinoline (150 mg, 0.66 mmol) and CDI (214 mg, 1.3 mmol). The resulting reaction mixture was stirred at 60° C. for 2 h and then diluted with ethyl acetate (60 mL) and water (30 mL). The aqueous layer was extracted with ethyl acetate (3×40 mL). The combined organic layers were washed with brine (30 mL), dried over Na₂SO₄, filtered and concentrated under reduced pressure to give a residue which was purified by column chromatography to give tert-butyl 7-((S)-1-(4-fluorophenyl)-1,2,3,4-tetrahydroisoquinoline-2-carboxamido)-5-azaspiro[2.4]heptane-5-carboxylate.

LCMS: (M+H)⁺=465; Retention time=1.888 min. LCMS CP Method F

Step 2: To a solution of tert-butyl 7-((S)-1-(4-fluorophenyl)-1,2,3,4-tetrahydroisoquinoline-2-carboxamido)-5-azaspiro[2.4]heptane-5-carboxylate (150 mg, 0.3 mmol) in DCM (1 mL) was added TFA (1 mL) at 0° C. and the reaction mixture was stirred at room temperature for 2 h. Then the mixture was concentrated under reduced pressure. The residue was purified by column chromatography to give 1-(4-fluorphenyl-N-(4-azaspiro[2.4]heptan-7-yl)-3,4-dihydroisoquinoline-2(1H)-carboxamide.

LCMS: (M+H)⁺=366; Retention time=1.511 min. LCMS CP Method F

Step 3: To a mixture of 1-(4-fluorophenyl)-N-(5-azaspiro[2.4]heptan-7-yl)-3,4-dihydroisoquinoline-2(1H-carboxamide (100 mg, 0.27 mmol) and 37% CH₂O (44 mg, 0.54 mmol) in MeOH (2 mL) was added NaBH₃CN (34 mg, 0.54 mmol) at 0° C. and the mixture was stirred at room temperature for 2 h. Then the mixture was concentrated under reduced pressure. The residue was purified by Prep-HPLC to give 1-(4-fluorophenyl)-N-(5-methyl-5-azaspiro[2.4]heptan-7-yl)-3,4-dihydroisoquinoline-2(1H-carboxamide.

LCMS: (M+H)⁺=380; Retention time=1.522 min. LCMS CP Method F

The diastereomers were separated by chiral HPLC eluting with n-Hexane (0.1% DEA): EtOH (0.1% DEA) over an EnantioPak® AD column (4.6*250 mm 5 μm) to give COMPOUND 2315 (6.6 mg, retention time=1.091 min), COMPOUND 2316 (14.9 mg, retention time=2.451 min), COMPOUND 2317 (11.2 mg, retention time=0.708 min) and COMPOUND 2318 (9.1 mg, retention time=1.466 min). The stereochemistry at both the pyrrolidine and THIQ chiral centers is arbitrarily assigned based on chromatographic elution order as compared to related analogues of known configuration.

COMPOUND 2315: LCMS: (M+H)⁺=380; (214 nm); retention time=1.493 min. LCMS CP Method F

Chiral SFC: CO₂/MeOH containing 0.2% MA (65%:35%) over a CHIRALPAK® IG column (4.6*100 mm 5 μm), retention time=1.981 min.

COMPOUND 2316: LCMS: (M+H)⁺=380; (214 nm); retention time=1.509 min. LCMS CP Method F

Chiral SFC: CO₂/MeOH containing 0.2% MA (65%:35%) over a CHIRALPAK® IG column (4.6*100 mm 5 μm), retention time=2.267 min.

COMPOUND 2317: LCMS: (M+H)⁺=380; (214 nm); retention time=1.499 min. LCMS CP Method F

Chiral SFC: CO₂/MeOH containing 0.2% MA (65%:35%) over a CHIRALPAK® IG column (4.6*100 mm 5 μm), retention time=1.064 min.

COMPOUND 2318: LCMS: (M+H)⁺=380; (214 nm); retention time=1.489 min. LCMS CP Method F

Chiral SFC: CO₂/MeOH containing 0.2% MA (65%:35%) over a CHIRALPAK® IG column (4.6*100 mm 5 μm), retention time=2.684 min.

Synthesis of COMPOUND 2364

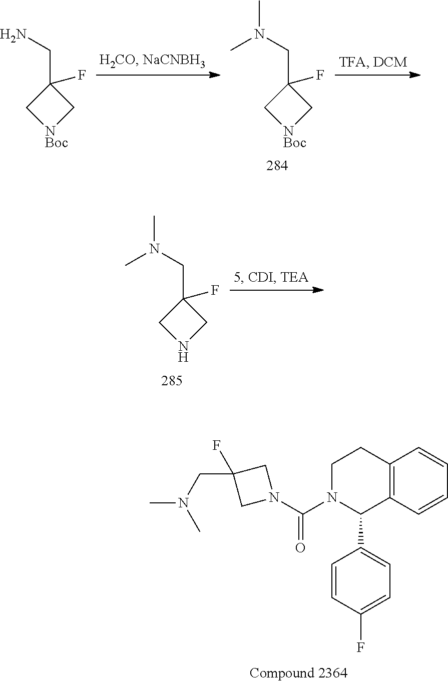

Step 1 To a solution of tert-butyl 3-(aminomethyl)-3-fluoroazetidine-1-carboxylate (204 mg, 1.0 mmol) and formaldehyde (35% w/w %) (900 mg, 10 mmol) in MeOH (5 mL) was added sodium cyanoborohydride (252 mg, 4.0 mmol) and the resulting mixture was stirred at 30° C. for 3 h. Then the mixture was concentrated and the residue was diluted with water (10 mL). The mixture was extracted with EA (3×10 mL). The combined organic layers were washed with brine (20 mL), dried over anhydrous Na₂SO₄, filtered and concentrated to give the product which was used directly in the next step reaction without further purification.

LCMS: (M+H)⁺=233, (214 nm); Retention time=1.39 min. LCMS CP Method C1

Step 2 To a solution of tert-butyl 3-((dimethylamino)methyl)-3-fluoroazetidine-1-carboxylate (200 mg) in DCM (2 mL) was added TFA (0.2 mL) and the resulting reaction mixture was stirred at room temperature for 1 h. Then the mixture was concentrated to give a crude oil (100 mg) which was used directly in the next step reaction without further purification.

LCMS: (M+H)⁺=133, (ELSD); Retention time=0.34 min. LCMS CP Method C1

Step 3 To a solution of 1-(3-fluoroazetidin-3-yl)-N,N-dimethylmethanamine (100 mg, 0.76 mmol) in DCM (3 mL) was added triphosgene (77 mg, 0.26 mmol) and the reaction mixture was stirred at 0° C. for 1 h. Then the mixture was concentrated to give a white solid which was redissolved in DMF (3 mL). To the DMF solution were added (S)-1-(4-fluorophenyl)-1, 2, 3, 4-tetrahydroisoquinoline (174 mg, 0.76 mmol) and TEA (154 mg, 1.52 mmol) and the mixture was stirred at 40° C. overnight. The mixture was cooled to room temperature and water (10 mL) was added. The mixture was extracted with ethyl acetate (3×10 mL) and the combined organic layers washed with brine (3×10 mL), dried over Na₂SO₄, filtered, and concentrated in vacuo to give the crude product. This was purified by Prep-HPLC to give (S)-(3-((dimethylamino)methyl)-3-fluoroazetidin-1-yl)(1-(4-fluorophenyl)-3,4-dihydroisoquinolin-2(1H)-yl)methanone.

COMPOUND 2364: LCMS: (M+H)⁺=386; (214 nm); Retention time=1.48 min. LCMS CP Method C1

Chiral SFC: CO₂/MeOH containing 0.2% MA (65%:35%) over CHIRALPAK® IG column (4.6*100 mm 5 μm), retention time=2.51 min).

Synthesis of COMPOUND 2367

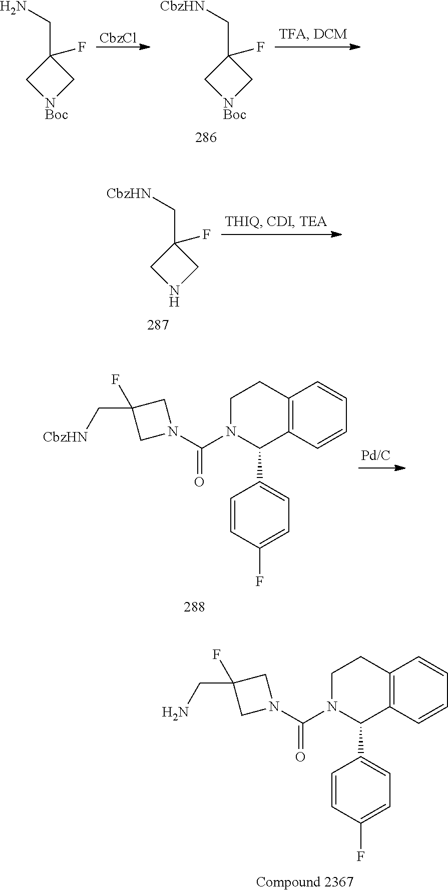 [SMILES: "CbzCl" ; CC(C)(C)OC(=O)N1CC(F)(CN)C1 ; CC(C)(C)OC(=O)N1CC(F)(CNC(=O)OCc2ccccc2)C1 ; O=C(NCC1(F)CNC1)OCc1ccccc1 ; O=C(NCC1(F)CN(C(=O)N2CCc3ccccc3[C@@H]2c2ccc(F)cc2)C1)OCc1ccccc1 ; NCC1(F)CN(C(=O)N2CCc3ccccc3[C@@H]2c2ccc(F)cc2)C1]

Step 1 To a solution of tert-butyl 3-(aminomethyl)-3-fluoroazetidine-1-carboxylate (612 mg, 3 mmol) and TEA (909 mg, 9 mmol) in DCM (10 mL) was added benzyl carbonochloridate (612 mg, 3.6 mmol) and the resulting mixture was stirred at 25° C. for 5 h. Water (10 mL) was added and the mixture extracted with DCM (3×10 mL). The combined organic layers were washed with brine (20 mL), dried over anhydrous Na₂SO₄, filtered and concentrated to give a light yellow oil which was purified by Prep-HPLC to give tert-butyl 3-((benzyloxycarbonylamino)methyl)-3-fluoroazetidine-1-carboxylate.

LCMS: (M+H)⁺=339, (214 nm); Retention time=1.62 min. LCMS CP Method C1

Step 2 To a solution of tert-butyl 3-((benzyloxycarbonylamino)methyl)-3-fluoroazetidine-1-carboxylate (460 mg) in DCM (10 mL) was added TFA (2 mL) and the resulting mixture was stirred at room temperature for 1 h. Then the mixture was concentrated under reduced pressure to give a crude product which was used directly in the next step reaction without further purification.

LCMS: (M+H)⁺=239, (214 nm); Retention time=0.95 min. LCMS CP Method C1

Step 3 To a solution of (S)-1-(4-fluorophenyl)-1, 2, 3, 4-tetrahydroisoquinoline (260 mg, 1.15 mmol) in DCM (3 mL) was added triphosgene (119 mg, 0.4 mmol) and the reaction mixture was stirred at 0° C. for 1 h. Then the mixture was concentrated to give a white solid which was redissolved in DMF (3 mL). To this solution were added benzyl (3-fluoroazetidin-3-yl)methylcarbamate (220 mg, 0.92 mmol) and TEA (279 mg, 2.76 mmol) and the resulting mixture was stirred at 40° C. overnight. The mixture was cooled to room temperature and water (10 mL) was added to stop the reaction and the mixture was extracted with ethyl acetate (3×10 mL). The combined organic layers were washed with brine (3×10 mL), dried over Na₂SO₄, filtered and concentrated in vacuo to give a residue which was purified by Prep-HPLC to give (S)-benzyl (3-fluoro-1-(1-(4-fluorophenyl)-1,2,3,4-tetrahydroisoquinoline-2-carbonyl)azetidin-3-yl) methylcarbamate.

LCMS: (M+H)⁺=492, (214 nm); Retention time=1.75 min. LCMS CP Method C1

Step 4 To a solution of (S)-benzyl (3-fluoro-1-(1-(4-fluorophenyl)-1, 2, 3, 4-tetrahydroisoquinoline-2-carbonyl)azetidin-3-yl) methylcarbamate (50 mg, 0.1 mmol) in MeOH (3 mL) was added Pd/C (20 mg, 10 wt %) and the reaction mixture was stirred at 25° C. for 2 h under hydrogen atmosphere. Then the mixture was filtered through celite and the filtrate concentrated to give a crude oil which was purified by Prep-HPLC to give (S)-(3-(aminomethyl)-3-fluoroazetidin-1-yl)(1-(4-fluorophenyl)-3,4-dihydroisoquinolin-2(1H)-yl)methanone.

COMPOUND 2367: LCMS: (M+H)⁺=357; (214 nm); Retention time=1.43 min. LCMS CP Method C1

Chiral SFC: CO₂/MeOH containing 0.2% MA (60%:40%) over CHIRALPAK® IG column (4.6*100 mm 5 μm), retention time=2.845 min).

Synthesis of COMPOUND 2368 and COMPOUND 2369

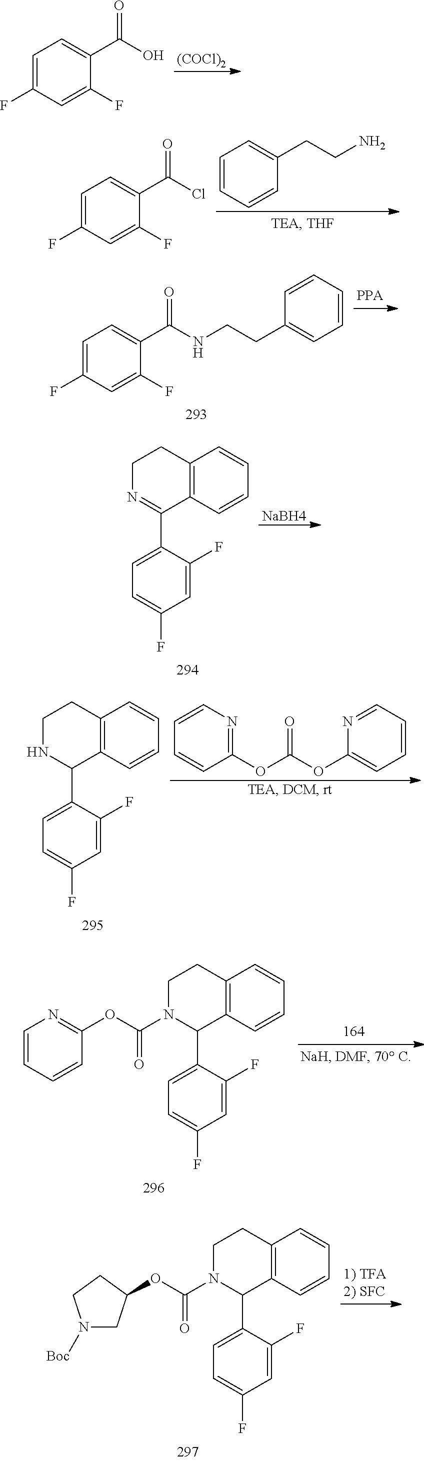

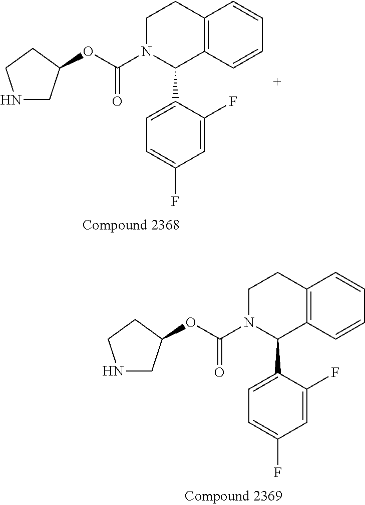 [SMILES: O=C(O[C@@H]1CCNC1)N1CCc2ccccc2[C@@H]1c1ccc(F)cc1F ; O=C(O[C@@H]1CCNC1)N1CCc2ccccc2[C@H]1c1ccc(F)cc1F]

Step 1: To a solution of 2,4-difluorobenzoic acid (5.0 g, 31.6 mmol) in anhydrous DCM (50 mL) was added dropwise oxalyl chloride (5.36 mL, 63.2 mmol) at 0° C. After addition, the reaction mixture was stirred at room temperature for 16 h and then concentrated to give crude 2,4-difluorobenzoyl chloride which was used directly in the next step reaction.

Step 2: To a solution of 2-phenylethan-1-amine (3.83 g, 31.6 mmol) and TEA (8.78 mL, 63.2 mmol) in tetrahydrofuran (60 mL) was added dropwise a solution of 2,4-difluorobenzoyl chloride (5.58 g, 31.6 mmol) in THF (30 mL) at 0° C. The reaction mixture was stirred at room temperature for 16 h and then poured into water and extracted with EtOAc (3×50 mL). The combined organic layers were dried over anhydrous Na₂SO₄, filtered and concentrated. The crude compound was purified by column chromatography on silica gel (PE/EtOAc, 1:10 to 1:4) to give 2,4-difluoro-N-phenethylbenzamide.

LCMS: (M+H)⁺=262; Retention time=1.759 min. LCMS CP Method C

Step 3: To a 100 mL flask containing PPA (20.0 g) was added 2,4-difluoro-N-phenethylbenzamide (5 g, 19.1 mmol) at 140° C. and the resulting reaction mixture was stirred at 150° C. for 4 h. Then the reaction mixture was cooled to 90° C. and poured into a cooled NaOH solution (130 mL, 7.5 M) and extracted with DCM (100 mL×3). The combined organic layers were washed with brine, dried over anhydrous Na₂SO₄, filtered and concentrated to give crude 1-(2,4-difluorophenyl)-3,4-dihydroisoquinoline which was used directly in the next step reaction without further purification.

LCMS: (M+H)⁺=244; Retention time=1.303 min. LCMS CP Method B

Step 4: To an ice-cold solution of 1-(2,4-difluorophenyl)-3,4-dihydroisoquinoline (4.5 g, 18.5 mmol) in MeOH (40 mL) was added slowly NaBH₄ (1.4 g, 37.0 mmol) at 0° C. The reaction mixture was stirred at room temperature for 2 h and then concentrated. The residue was suspended in water and the mixture was extracted with DCM (3×50 mL). The combined organic layers were dried over anhydrous Na₂SO₄, filtered and concentrated to give crude 1-(2,4-difluorophenyl)-1,2,3,4-tetrahydroisoquinoline which was used directly in the next step reaction without further purification.

LCMS: (M+H)⁺=246; Retention time=1.276 min. LCMS CP Method A1

Step 5: To a solution of 1-(2,4-difluorophenyl)-1,2,3,4-tetrahydroisoquinoline (500 mg, 2.038 mmol) in DCM (5 mL) were added di(pyridin-2-yl) carbonate (440 mg, 2.038 mmol) and TEA (0.57 mL, 4.08 mmol) at room temperature. The reaction mixture was stirred at room temperature for 2 h. Then water (10 mL) was added and the mixture was extracted with DCM (3×20 mL). The combined organic layers were dried over Na₂SO₄, filtered and concentrated in vacuum to give pyridin-2-yl 1-(2,4-difluorophenyl)-3,4-dihydroisoquinoline-2(1H)-carboxylate which was used directly in the next step reaction.

LCMS: (M+H)⁺=367; Retention time=1.817 min. LCMS CP Method B

Step 6: To a solution of tert-butyl (R)-3-hydroxypyrrolidine-1-carboxylate (325 mg, 1.73 mmol) in DMF (5 mL) was added NaH (163 mg, 3.46 mmol) at 0° C. After stirring for 30 min at room temperature, the reaction mixture was cooled to 0° C. again and a solution of pyridin-2-yl 1-(2,4-difluorophenyl)-3,4-dihydroisoquinoline-2(1H)-carboxylate (635 mg, 1.73 mmol) was added. Then the reaction mixture was heated to 70° C. and stirred at the same temperature for 1 h. The mixture was cooled, poured into water and extracted with DCM (3×50 mL). The combined organic layers were dried over Na₂SO₄, filtered and concentrated to give a residue which was purified by Prep-HPLC to give (R)-1-(tert-butoxycarbonyl)pyrrolidin-3-yl 1-(2,4-difluorophenyl)-3,4-dihydroisoquinoline-2(1H)-carboxylate.

LCMS: (M−55)⁺=403; Retention time=1.96 min. LCMS CP Method B

Step 7: To a solution of (R)-1-(tert-butoxycarbonyl)pyrrolidin-3-yl 1-(2,4-difluorophenyl)-3,4-dihydroisoquinoline-2(1H)-carboxylate (638 mg, 1.39 mmol) in DCM (10 mL) was added TFA (2 mL) at 0° C. and the mixture was stirred at room temperature for 1 h. Then the mixture was concentrated under reduced pressure to give a residue which was suspended in water. The suspension was adjusted to pH 8 with 1N NaOH and the mixture was extracted with DCM (3×30 mL). The combined organic layers were dried over Na₂SO₄, filtered and concentrated to give a residue which was purified by Prep-HPLC to give (R)-pyrrolidin-3-yl 1-(2,4-difluorophenyl)-3,4-dihydroisoquinoline-2(1H)-carboxylate.

LCMS: (M+H)⁺=359; Retention time=1.457 min. LCMS CP Method C1

The diastereomers (180 mg) were separated by chiral SFC eluting with CO₂/EtOH containing 0.5% methanolic ammonia over a Daicel® AD column (20×250 mm, 10 μm) to give COMPOUND 2368 and COMPOUND 2369. Stereochemical assignment of (R) at the pyrrolidine chiral center is based on enantiomerically pure starting materials; the configuration at the 1 position of the tetrahydroisoquinoline is arbitrarily assigned based on chromatographic elution order as compared to related analogues of known configuration.

Synthesis of Compounds 2375 and 2374

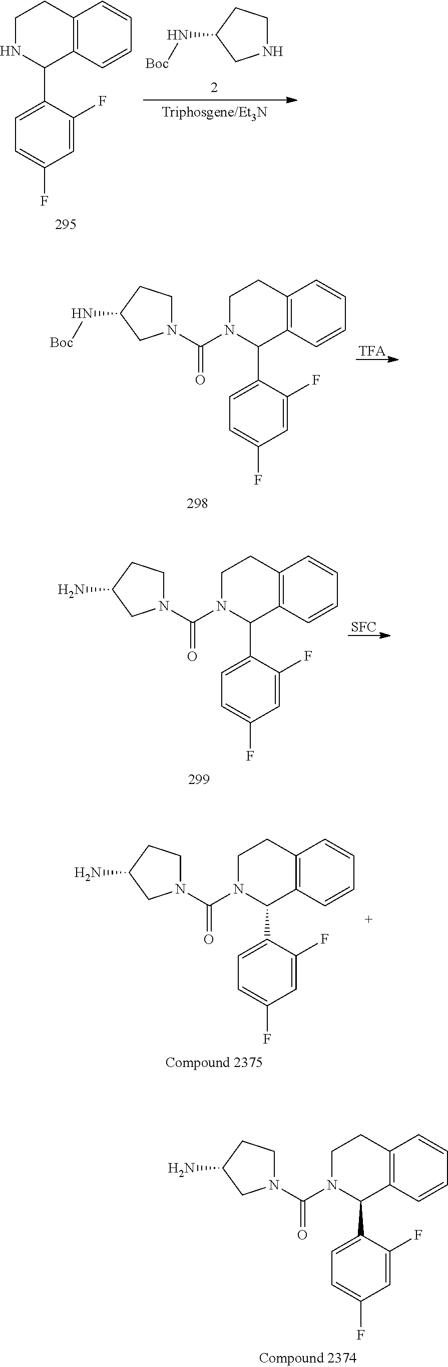

Step 1: To a solution of 1-(2,4-difluorophenyl)-1,2,3,4-tetrahydroisoquinoline (300 mg, 1.2 mmol) and Et₃N (240 mg, 2.4 mmol) in DCM (4 mL) under a nitrogen atmosphere was slowly added triphosgene (118 mg, 0.3 mmol). The reaction mixture was stirred at room temperature for one hour and then the solvent was removed under reduced pressure to give crude 1-(2,4-difluorophenyl)-3,4-dihydroisoquinoline-2(1H)-carbonyl chloride. To a solution of (R)-tert-butyl pyrrolidin-3-ylcarbamate (228 mg, 1.2 mmol) and Et₃N (124 mg, 1.2 mmol) in DCM (4 mL) was added a solution of 1-(2,4-difluorophenyl)-3,4-dihydroisoquinoline-2(1H)-carbonyl chloride (368 mg, 1.2 mmol) in DCM (2 mL) and the mixture was stirred at room temperature for 2 h. Water was (20 mL) added to quench the reaction and the mixture was extracted with DCM (3×20 mL). The combined organic layers were dried over Na₂SO₄, filtered and concentrated to give tert-butyl (R)-1-(1-(2,4-difluorophenyl)-1,2,3,4-tetrahydroisoquinoline-2-carbonyl)pyrrolidin-3-ylcarbamate which was used directly in the next step reaction without further purification.

LCMS: (M+H)⁺=458; Retention time=2.10 min. LCMS CP Method A

Step 2: To a solution of tert-butyl (R)-1-(1-(2,4-difluorophenyl)-1,2,3,4-tetrahydroisoquinoline-2-carbonyl)pyrrolidin-3-ylcarbamate (450 mg, 0.98 mmol) in DCM (2 mL) was added TFA (1 mL). The reaction mixture was stirred at room temperature for 2 h and then concentrated. The residue was purified by Prep-HPLC to give ((R)-3-aminopyrrolidin-1-yl)(1-(2,4-difluorophenyl)-3,4-dihydroisoquinolin-2(1H)-yl)methanone.

LCMS: (M+H)⁺=358; Retention time=1.45 min. LCMS CP Method C

Synthesis of Compound 2376

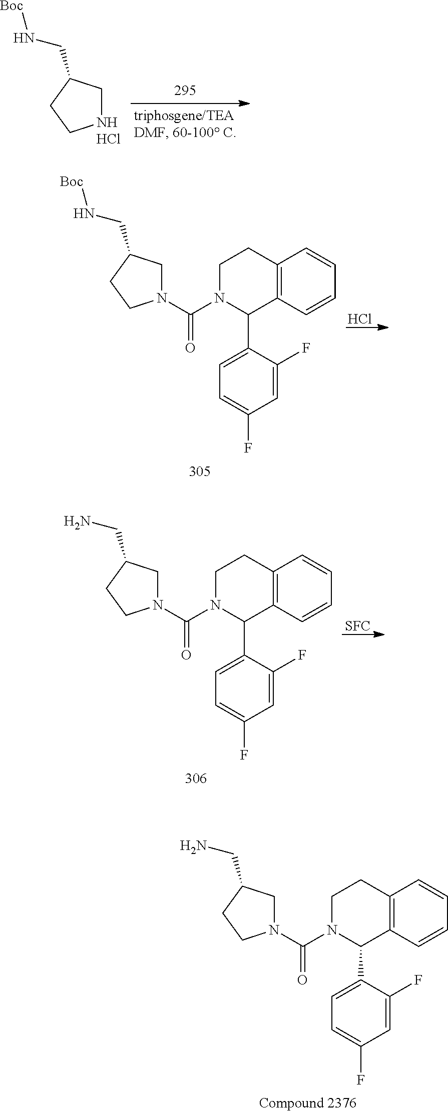

Step 1 To a solution of 1-(2, 4-difluorophenyl)-1,2,3,4-tetrahydroisoquinoline (250 mg, 1.1 mmol) in DCM (3 mL) was added triphosgene (130 mg, 0.44 mmol). The resulting reaction mixture was stirred for 30 min at 0° C. and then concentrated to give a white solid which was added to a solution of tert-butyl (S)-(pyrrolidin-3-ylmethyl)carbamate (236 mg, 1 mmol) and TEA (333 mg, 3.3 mmol) in DMF (10 mL). The reaction mixture was stirred at 60° C. for 3 h and then diluted with water (50 mL). The mixture was extracted with EA (3×30 mL). The combined organic layers were washed with brine (50 mL), dried over anhydrous Na₂SO₄, filtered and concentrated to give a light yellow oil which was purified by Prep-HPLC to give tert-butyl ((3R)-1-(1-(2,4-difluorophenyl)-1,2,3,4-tetrahydroisoquinoline-2-carbonyl)pyrrolidin-3-yl)methylcarbamate.

LCMS: (M+H)⁺=454, (214 nm); Retention time=1.70 min. LCMS CP Method C1

Step 2 To a 100 mL flask containing tert-butyl ((3R)-1-(1-(2,4-difluorophenyl)-1,2,3,4-tetrahydroisoquinoline-2-carbonyl)pyrrolidin-3-yl)methylcarbamate (100 mg, 0.21 mmol) was added a solution of HCl in 1,4-dioxane (4.0 M, 5 mL) and the resulting reaction mixture was stirred at room temperature for 1 h. Then the mixture was concentrated to give a crude oil which was purified by Prep-HPLC to give ((R)-3-(aminomethyl)pyrrolidin-1-yl)(1-(2,4-difluorophenyl)-3,4-dihydroisoquinolin-2(1H)-yl)methanone.

LCMS: (M+H)⁺=354, (214 nm); Retention time=1.38 min. LCMS CP Method C1

Synthesis of COMPOUND 2370 and COMPOUND 2371

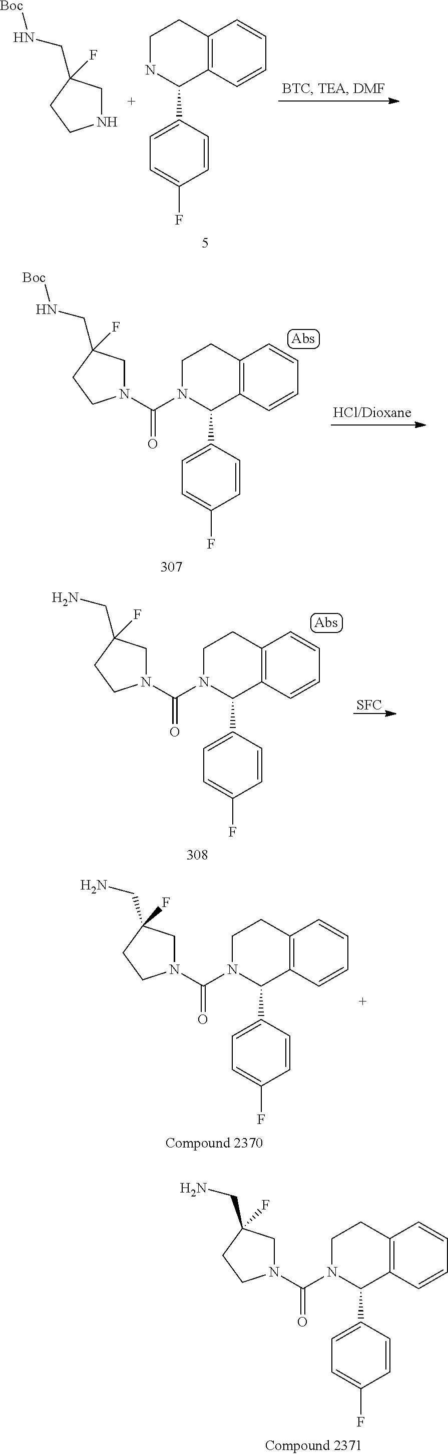

Step 1 To a solution of (S)-1-(4-fluorophenyl)-1, 2, 3, 4-tetrahydroisoquinoline (454 mg, 2 mmol) in DCM (10 mL) was added triphosgene (237 mg, 0.8 mmol) and the reaction mixture was stirred at 0° C. for 30 min. Then the mixture was concentrated to give a white solid which was dissolved in DMF (5 mL). To this DMF solution were added tert-butyl ((3-fluoropyrrolidin-3-yl)methyl)carbamate (234 mg, 1.07 mmol) and TEA (325 mg, 3.21 mmol) and the reaction mixture was stirred at 60° C. for 2 h. Then the mixture was cooled to room temperature, water (15 mL) was added and the mixture extracted with ethyl acetate (3×15 mL). The combined organic layers were washed with brine (3×10 mL), dried over Na₂SO₄, filtered and concentrated in vacuo to give a residue which was purified by Prep-HPLC to give tert-butyl ((3-fluoro-1-((S)-1-(4-fluorophenyl)-1,2,3,4-tetrahydroisoquinoline-2-carbonyl)pyrrolidin-3-yl)methyl)carbamate.

LCMS: (M+H)⁺=472, (214 nm); Retention time=2.01 min. LCMS CP Method C1

Step 2 To a round bottomed flask containing a solution of HCl in Dioxane (4.0 M, 10 mL) was added tert-butyl ((3-fluoro-1-((S)-1-(4-fluorophenyl)-1,2,3,4-tetrahydroisoquinoline-2-carbonyl)pyrrolidin-3-yl)methyl)carbamate (403 mg, 0.85 mmol) and the reaction mixture was stirred at room temperature for 1 h. Then the mixture was concentrated to give a crude oil which was purified by Prep-HPLC to give (3-(aminomethyl)-3-fluoropyrrolidin-1-yl)((S)-1-(4-fluorophenyl)-3,4-dihydroisoquinolin-2(1H)-yl)methanone.

Synthesis of COMPOUND 2270)

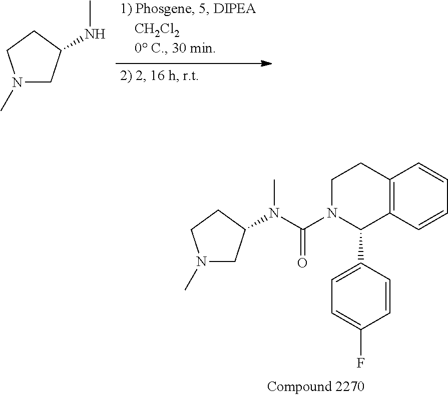

At 0° C., a solution of (S)-1-(4-fluorophenyl)-1,2,3,4-tetrahydroisoquinoline (50 mg, 0.220 mmol) and N,N-diisopropylethylamine (0.084 mL, 0.484 mmol) in dichloromethane (0.4 mL) was added dropwise to a solution of phosgene (20 wt % in toluene, 0.255 mL, 0.484 mmol) in dichloromethane (0.4 mL) over 5 minutes. After 30 minutes, the reaction mixture was allowed to warm to room temperature and methyl-((S)-1-methyl-pyrrolidin-3-yl)-amine (37.7 mg, 0.330 mmol) was added. After 16 hours, the reaction mixture was concentrated and purified by flash column chromatography (silica, 0 to 5% 7 M ammonia in methanol/dichloromethane) and by basic preparative MPLC (Linear Gradient: t=0 min 5% A, t=1 min 5% A; t=2 min 10%; t=17 min 50% A; t=18 min 100%; t=23 min 100% A; detection: 220 nm). The product was lyophilized from a mixture of acetonitrile and water (1:1, 4 mL) to give (S)-1-(4-fluorophenyl)-N-methyl-N—((S)-1-methylpyrrolidin-3-yl)-3,4-dihydroisoquinoline-2(1)-carboxamide (COMPOUND 2270). LCMS: 97%, RT=1.10 min., (M+H)⁺=368 (method P).

Compound 2265 was prepared by a similar synthesis as for Compound 2270.

COMPOUND 2265, (M+H)⁺=368. RT=1.17 min. (method Q). (S)-(1,4-diazepan-1-yl)(1-(4-fluorophenyl)-3,4-dihydroisoquinolin-2(1H-yl)methanone (COMPOUND 2264)

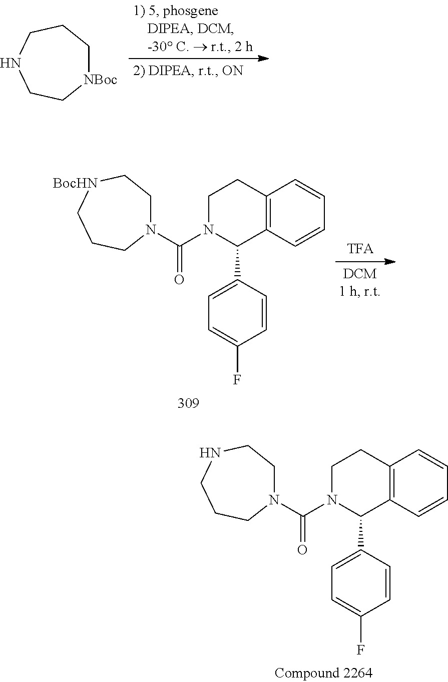

Synthesis of tert-butyl (S)-4-(1-(4-fluorophenyl)-1,2,3,4-tetrahydroisoquinoline-2-carbonyl)-1,4-diazepane-1-carboxylate.

Starting from (S)-1-(4-fluorophenyl)-1,2,3,4-tetrahydroisoquinoline (645 mg, 2.84 mmol) and tert-butyl 1,4-diazepane-1-carboxylate (568 mg, 2.84 mmol), tert-butyl (S)-4-(1-(4-fluorophenyl)-1,2,3,4-tetrahydroisoquinoline-2-carbonyl)-1,4-diazepane-1-carboxylate (821 mg) was prepared according to the procedure described for (S)-1-(4-fluorophenyl)-N-methyl-N—((S)-1-methylpyrrolidin-3-yl)-3,4-dihydroisoquinoline-2(1H)-carboxamide (COMPOUND 2270). Acidic workup (aqueous HCl (1 M)/dichloromethane) was followed by concentration and purification by flash column chromatography (silica, 0 to 50% ethyl acetate in heptane) LCMS: 96%, RT=2.00 min., (M+H)⁺=454 (method P).

Synthesis of (S)-(1,4-diazepan-1-yl)(1-(4-fluorophenyl)-3,4-dihydroisoquinolin-2(1H)-yl)methanone (COMPOUND 2264).

Starting from tert-butyl (S)-4-(1-(4-fluorophenyl)-1,2,3,4-tetrahydroisoquinoline-2-carbonyl)-1,4-diazepane-1-carboxylate (821 mg, 1.81 mmol), (S)-(1,4-diazepan-1-yl)(1-(4-fluorophenyl)-3,4-dihydroisoquinolin-2(1H-yl)methanone (COMPOUND 2264) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H-carboxamide 2,2,2-trifluoroacetate (COMPOUND 2201). Purification by acidic preparative MPLC (Linear Gradient: t=0 min 5% A, t=1 min 5% A; t=2 min 10% A; t=22 min 50% A; t=23 min 100% A; t=28 min 100% A; detection 220/280 nm) was followed by desalting (SCX-2 (5 g) ion exchange chromatography). LCMS: RT=1.05 min., (M+H)⁺=354 (method P).

Compound 2262 was prepared by a similar synthesis as for Compound 2264.

COMPOUND 2262 (M−Cl)⁻=390. RT=2.80 min., (method AK)

### (S)-(1-(4-fluorophenyl)-3,4-dihydroisoquinolin-2(1H)-yl)(4-methyl-1,4-diazepan-1-yl)methanone (Compound 2269)

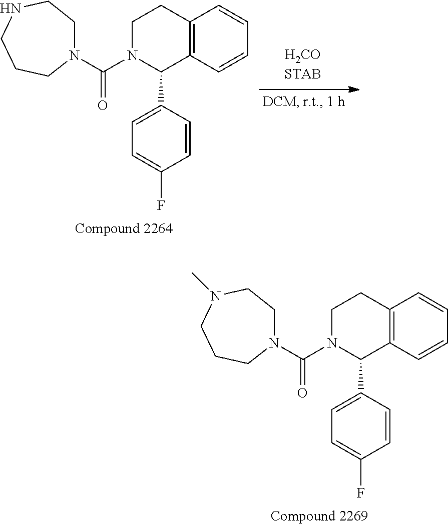 [SMILES: O=C(N1CCCNCC1)N1CCc2ccccc2[C@@H]1c1ccc(F)cc1 ; CN1CCCN(C(=O)N2CCc3ccccc3[C@@H]2c2ccc(F)cc2)CC1]

Synthesis of (S)-(1-(4-fluorophenyl)-3,4-dihydroisoquinolin-2(1H)-yl)(4-methyl-1,4-diazepan-1-yl)methanone (COMPOUND 2269).

Formaldehyde (37 wt % solution in water stabilized with 10-15% methanol, 73 μL, 0.97 mmol) was added to a solution of (S)-(1,4-diazepan-1-yl)(1-(4-fluorophenyl)-3,4-dihydroisoquinolin-2(1H-yl)methanone (COMPOUND 2264, 0.17 g, 0.48 mmol) in dichloromethane (2.5 mL). Following the addition of sodium triacetoxyborohydride (0.21 g, 0.97 mmol), the reaction mixture was stirred vigorously at room temperature for 1 hour. The reaction mixture was diluted with dichloromethane (10 mL) and washed with a mixture of water and saturated aqueous K₂CO₃ (1:1, 10 mL). The layers were separated using a phase separator and the organic filtrate was evaporated under reduced pressure. The residue was dissolved in methanol (2 mL), loaded onto an SCX-2 column (2 g) and eluted with methanol until the effluent was neutral. Next, the column was eluted with ammonia in methanol (1.5 M) and the basic fraction was concentrated to dryness under reduced pressure to give (S)-(1-(4-fluorophenyl)-3,4-dihydroisoquinolin-2(1H-yl)(4-methyl-1,4-diazepan-1-yl)methanone (COMPOUND 2269). LCMS: RT=1.06 min., (M+H)⁺=368 (method P).

### (S)-(6,6-difluoro-4-methyl-1,4-diazepan-1-yl)(1-(4-fluorophenyl)-3,4-dihydroisoquinolin-2(1H-yl)methanone hydrochloride (Compound 2261)

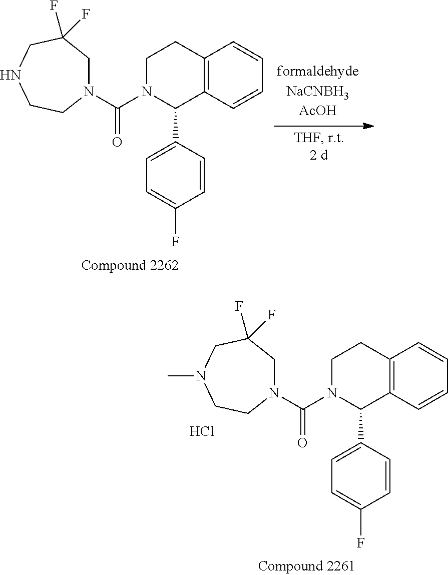 [SMILES: O=C(N1CCNCC(F)(F)C1)N1CCc2ccccc2[C@@H]1c1ccc(F)cc1 ; CN1CCN(C(=O)N2CCc3ccccc3[C@@H]2c2ccc(F)cc2)CC(F)(F)C1 ; Cl]

Synthesis of (S)-(6,6-difluoro-4-methyl-1,4-diazepan-1-yl)(1-(4-fluorophenyl)-3,4-dihydroisoquinolin-2(1H)-yl)methanone hydrochloride (COMPOUND 2261).

Sodium cyanoborohydride (39 mg, 0.621 mmol), followed by formaldehyde (37 wt % in water, 56 μL, 0.752 mmol) and acetic acid (13 μL, 0.227 mmol) were added to a solution of (S)-(6,6-difluoro-1,4-diazepan-1-yl)(1-(4-fluorophenyl)-3,4-dihydroisoquinolin-2(1H)-yl)methanone (COMPOUND 2262, 59 mg, 0.152 mmol) in tetrahydrofuran (1.5 mL). After stirring for 2 days, another portion of sodium cyanoborohydride (38 mg, 0.605 mmol), formaldehyde (37 wt % in water, 56 μL, 0.752 mmol), and acetic acid (13 μL, 0.227 mmol) were added and stirring was continued for 6 hours. Then, methanol (1 mL) was added and the reaction mixture was evaporated under reduced pressure. The residue was partitioned between saturated aqueous NaHCO₃ (5 mL) and dichloromethane (10 mL). The layers were separated using a phase-separator and the organic filtrate was evaporated under reduced pressure. The residue was dissolved in methanol (1 mL) and brought onto an SCX-2 column (1 g) and eluted with methanol until neutral. Next, the column was eluted with ammonia in methanol (1 M). The basic fraction was concentrated to dryness under reduced pressure. The residue was taken up in a mixture of acetonitrile and water (1:1, 4 mL), aqueous HCl (1.00 M, 134 μL, 0.134 mmol) was added, and the mixture was lyophilized to give (S)-(6,6-difluoro-4-methyl-1,4-diazepan-1-yl)(1-(4-fluorophenyl)-3,4-dihydroisoquinolin-2(1H-yl)methanone hydrochloride (COMPOUND 2261). LCMS: RT=1.40 min., (M−Cl)⁺=404 (method P).

### (R)-1-(4-fluorophenyl)-N—((R)-1,4-oxazepan-6-yl)-3,4-dihydroisoquinoline-2(1H-carboxamide (Compound 2275); (S)-1-(4-fluorophenyl)-N—((S)-1,4-oxazepan-6-yl)-3,4-dihydroisoquinoline-2(1H-carboxamide (Compound 2276); (S)-1-(4-fluorophenyl)-N—((R)-1,4-oxazepan-6-yl)-3,4-dihydroisoquinoline-2(1H-carboxamide (Compound 2277); (R)-1-(4-fluorophenyl)-N—((S)-1,4-oxazepan-6-yl)-3,4-dihydroisoquinoline-2(1H-carboxamide (compound 2278)

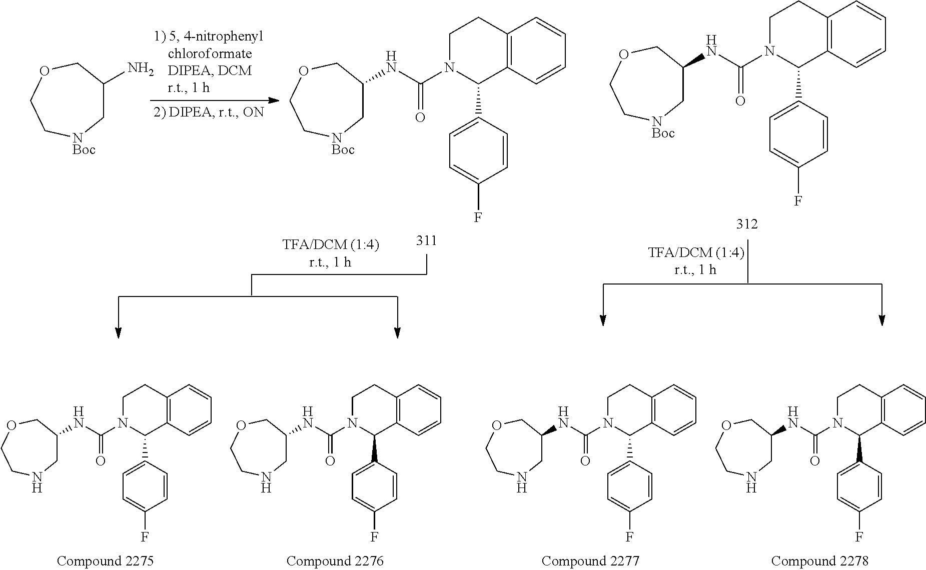

Synthesis of tert-butyl (S)-6-((S)-1-(4-fluorophenyl)-1,2,3,4-tetrahydroisoquinoline-2-carboxamido)-1,4-oxazepane-4-carboxylate and tert-butyl (R)-6-((S)-1-(4-fluorophenyl)-1,2,3,4-tetrahydroisoquinoline-2-carboxamido)-1,4-oxazepane-4-carboxylate.

Starting from tert-butyl 6-amino-1,4-oxazepane-4-carboxylate (628 mg, 2.90 mmol) and (S)-1-(4-fluorophenyl)-1,2,3,4-tetrahydroisoquinoline (792 mg, 3.48 mmol), tert-butyl (S)-6-((S)-1-(4-fluorophenyl)-1,2,3,4-tetrahydroisoquinoline-2-carboxamido)-1,4-oxazepane-4-carboxylate (257 mg) as the first SFC eluting isomer and tert-butyl (R)-6-((S)-1-(4-fluorophenyl)-1,2,3,4-tetrahydroisoquinoline-2-carboxamido)-1,4-oxazepane-4-carboxylate (271 mg) as the second SFC eluting isomer were prepared according to the procedure described for tert-butyl (cis-3-((S)-1-(4-fluorophenyl)-1,2,3,4-tetrahydroisoquinoline-2-carboxamido)cyclobutyl)carbamate (see COMPOUND 2236). The isomers were purified by acidic preparative MPLC (Linear Gradient: t=0 min 5% A, t=1 min 5% A; t=2 min 20% A; t=17 min 60% A; t=22 min 60% A; t=23 min 100% A; t=31 min 100% A; detection 220/254/280 nm) and preparative SFC (method AL). The absolute stereochemistry of both the oxazepane and tetrahydroisoquinoline moieties were arbitrarily assigned. First eluting isomer: LCMS: RT=1.91 min., (M+H)⁺=470 (method Q). SFC: RT=3.51 min., (M+H)⁺=470 (method F). Second eluting isomer: LCMS: RT=1.88 min., (M+H)⁺=470 (method Q). SFC: RT=5.85 min., (M+H)⁺=470 (method F).

Synthesis of (R)-1-(4-fluorophenyl)-N—((R)-1,4-oxazepan-6-yl)-3,4-dihydroisoquinoline-2(1H)-carboxamide (COMPOUND 2275) and (S)-1-(4-fluorophenyl)-N—((R)-1,4-oxazepan-6-yl)-3,4-dihydroisoquinoline-2(1H)-carboxamide (COMPOUND 2277).

Starting from tert-butyl (R)-6-((S)-1-(4-fluorophenyl)-1,2,3,4-tetrahydroisoquinoline-2-carboxamido)-1,4-oxazepane-4-carboxylate (0.27 g, 0.58 mmol), (R)-1-(4-fluorophenyl)-N—((R)-1,4-oxazepan-6-yl)-3,4-dihydroisoquinoline-2(1H)-carboxamide (COMPOUND 2275) as the first eluting and (S)-1-(4-fluorophenyl)-N—((R)-1,4-oxazepan-6-yl)-3,4-dihydroisoquinoline-2(1H-carboxamide (COMPOUND 2277) as the second eluting isomer were prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H-carboxamide 2,2,2-trifluoroacetate (COMPOUND 2201). After desalting by extraction from saturated aqueous NaHCO₃/dichloromethane and purification by preparative chiral HPLC (method AJ), the materials were lyophilized from a mixture of acetonitrile and water (1:1). The absolute stereochemistry of both the oxazepane and tetrahydroisoquinoline moieties were arbitrarily assigned First eluting isomer: LCMS: RT=2.62 min., (M+H)⁺=370 (method AK). Chiral LC: RT=17.71 min. (method AB). Second eluting isomer: LCMS: RT=2.62 min., (M+H)⁺=370 (method AK). Chiral LC: RT=25.57 min. (method AB).

Synthesis of (S)-1-(4-fluorophenyl)-N—((S)-1,4-oxazepan-6-yl)-3,4-dihydroisoquinoline-2(1H)-carboxamide (COMPOUND 2276) and (R)-1-(4-fluorophenyl)-N—((S)-1,4-oxazepan-6-yl)-3,4-dihydroisoquinoline-2(1H)-carboxamide (COMPOUND 2278).

Starting from tert-butyl (S)-6-((S)-1-(4-fluorophenyl)-1,2,3,4-tetrahydroisoquinoline-2-carboxamido)-1,4-oxazepane-4-carboxylate (0.25 g, 0.54 mmol), (S)-1-(4-fluorophenyl)-N—((S)-1,4-oxazepan-6-yl)-3,4-dihydroisoquinoline-2(1H)-carboxamide (COMPOUND 2276) as the first eluting isomer and (R)-1-(4-fluorophenyl)-N—((S)-1,4-oxazepan-6-yl)-3,4-dihydroisoquinoline-2(1H-carboxamide (COMPOUND 2278) as the second eluting isomer were prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H-carboxamide 2,2,2-trifluoroacetate (COMPOUND 2201). After desalting by extraction from saturated aqueous NaHCO₃/dichloromethane and purification by preparative chiral HPLC (method AJ), the materials were lyophilized from a mixture of acetonitrile and water (1:1). The absolute stereochemistry of both the oxazepane and tetrahydroisoquinoline moieties were arbitrarily assigned. First eluting isomer: LCMS: RT=1.03 min., (M+H)⁺=370 (method P). Chiral LC: RT=23.53 min. (method AB). Second eluting isomer: LCMS: RT=1.027 min., (M+H)⁺=370.2 (method P). Chiral LC: RT=51.282 min. (method AB).

### (S)-1-(4-fluorophenyl)-N—(((R)-2-oxooxazolidin-4-yl)methyl)-3,4-dihydroisoquinoline-2(1H)-carboxamide (Compound 2259)

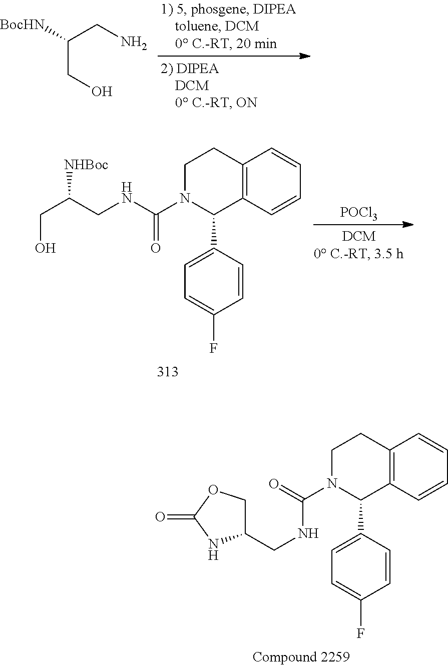

Synthesis of tert-butyl ((R)-1-((S)-1-(4-fluorophenyl)-1,2,3,4-tetrahydroisoquinoline-2-carboxamido)-3-hydroxypropan-2-yl)carbamate.

Starting from (S)-1-(4-fluorophenyl)-1,2,3,4-tetrahydroisoquinoline (98 mg, 0.431 mmol) and tert-butyl (R)-(1-amino-3-hydroxypropan-2-yl)carbamate (82 mg, 0.431 mmol), tert-butyl ((R)-1-((S)-1-(4-fluorophenyl)-1,2,3,4-tetrahydroisoquinoline-2-carboxamido)-3-hydroxypropan-2-yl)carbamate (165 mg (95 wt %)) was prepared as described for (S)-1-(4-fluorophenyl)-N-methyl-N—((S)-1-methylpyrrolidin-3-yl)-3,4-dihydroisoquinoline-2(1H)-carboxamide (COMPOUND 2270). After acidic workup (HCl (0.5 M)/heptane and ethyl acetate (1:1)) the organic layer underwent an additional wash (water and brine/NaHCO₃ (1:1)) and purification by flash column chromatography (silica, 40 to 100% ethyl acetate in heptane). LCMS: RT=2.01 min., (M+H)⁺=444 (method A).

Synthesis of (S)-1-(4-fluorophenyl)-N—(((R)-2-oxooxazolidin-4-yl)methyl)-3,4-dihydroisoquinoline-2(1H)-carboxamide (COMPOUND 2259).

At 0° C., phosphorus oxychloride (0.034 mL, 0.360 mmol) was added to a solution of tert-butyl ((R)-1-((S)-1-(4-fluorophenyl)-1,2,3,4-tetrahydroisoquinoline-2-carboxamido)-3-hydroxypropan-2-yl)carbamate (152 mg (95 wt %), 0.326 mmol) in tetrahydrofuran (dry, 5 mL). After 20 minutes, the mixture was warmed to room temperature. After 1 hour, pyridine (0.069 mL, 0.857 mmol) was added and the resulting suspension was diluted with tetrahydrofuran (dry, 2 mL) and dichloromethane (2 mL). After that, a second portion of phosphorus oxychloride (0.034 mL, 0.360 mmol) was added at 0° C. and the mixture was stirred at room temperature for 15 minutes. A third portion of phosphorus oxychloride (0.034 mL, 0.360 mmol) was added at 0° C. and the mixture was stirred at room temperature for 2 hours. The mixture was quenched with water and diluted with aqueous NaOH (0.2 M, 30 mL). Attempted extraction with dichloromethane (50 mL) failed upon which the mixture was acidified with aqueous HCl (1 M) and further diluted with water. The mixture was extracted with dichloromethane (2×200 mL). The combined organic layers were washed with brine, dried over Na₂SO₄, and evaporated under reduced pressure. The residue was purified by basic preparative MPLC (Linear Gradient: t=0 min 10% A, t=1 min 10% A; t=2 min 40% A; t=17 min 80% A; t=18 min 100% A; t=23 min 100% A; detection: 210 nm) to give (S)-1-(4-fluorophenyl)-N—(((R)-2-oxooxazolidin-4-yl)methyl)-3,4-dihydroisoquinoline-2(1H-carboxamide (COMPOUND 2259). LCMS: RT=1.409 min., (M+H)⁺=370 (method P).

### (S)-1-(4-fluorophenyl)-N—((S)-1-methylpyrrolidin-3-yl)-3,4-dihydroisoquinoline-2(1H)-carboxamide (Compound 2237)

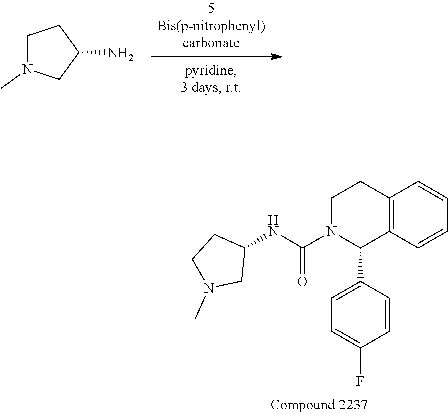

Synthesis of (S)-1-(4-fluorophenyl)-N—((S)-1-methylpyrrolidin-3-yl)-3,4-dihydroisoquinoline-2(1H)-carboxamide (COMPOUND 2237).

Starting from (S)-1-Methylpyrrolidin-3-amine (59.5 mg, 0.594 mmol) and (S)-1-(4-fluorophenyl)-1,2,3,4-tetrahydroisoquinoline (135 mg, 0.594 mmol), (S)-1-(4-fluorophenyl)-N—((S)-1-methylpyrrolidin-3-yl)-3,4-dihydroisoquinoline-2(1H)-carboxamide (COMPOUND 2237) was prepared as described for tert-butyl (S)-7-(benzyloxy)-1-(4-fluorophenyl)-3,4-dihydroisoquinoline-2(1H)-carboxylate (see COMPOUND 2115). LCMS: RT=1.56 min., (M+H)⁺=354 (method A).

Compound 2245 was prepared by a similar synthesis as for Compound 2237. COMPOUND 2245 (M+H)⁺=354. RT=2.64 min., (method AK)

### (S)-1-(4-fluorophenyl)-N-(1-methylpiperidin-4-yl)-3,4-dihydroisoquinoline-2(1H)-carboxamide (Compound 2238)

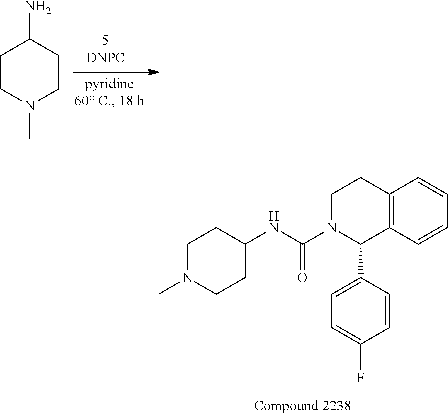

Synthesis of (S)-1-(4-fluorophenyl)-N-(1-methylpiperidin-4-yl)-3,4-dihydroisoquinoline-2(1H) carboxamide.

Starting from 1-methylpiperidin-4-amine (50 mg, 0.438 mmol) and (S)-1-(4-fluorophenyl)-1,2,3,4-tetrahydroisoquinoline (100 mg, 0.438 mmol), (S)-1-(4-fluorophenyl)-N-(1-methylpiperidin-4-yl)-3,4-dihydroisoquinoline-2(1H carboxamide (COMPOUND 2238) was prepared as described for tert-butyl (S)-7-(benzyloxy)-1-(4-fluorophenyl)-3,4-dihydroisoquinoline-2(1H)-carboxylate (see COMPOUND 2115) and purified by acidic preparative MPLC (Linear Gradient: t=0 min 5% A, t=1 min 5% A; t=16.6 min 40% A, t=17.6 min 100% A, t=22.8 min 100% A; detection: 210/220/280 nm). LCMS: RT=2.60 min., (M+H)⁺=368 (method AK).

### (S)—N-(azetidin-3-ylmethyl)-1-(4-fluorophenyl)-3,4-dihydroisoquinoline-2(1)-carboxamide acetate (Compound 2257)

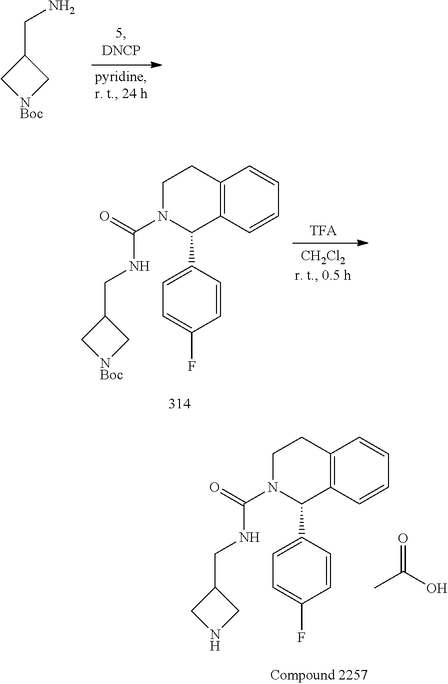

Synthesis of tert-butyl (S)-3-((1-(4-fluorophenyl)-1,2,3,4-tetrahydroisoquinoline-2-carboxamido)methyl)azetidine-1-carboxylate.

Starting from tert-butyl 3-(aminomethyl)azetidine-1-carboxylate (50 mg, 0.268 mmol) in pyridine (1 mL) and (S)-1-(4-fluorophenyl)-1,2,3,4-tetrahydroisoquinoline (61 mg, 0.27 mmol), (S)-3-((1-(4-fluorophenyl)-1,2,3,4-tetrahydroisoquinoline-2-carboxamido)methyl)azetidine-1-carboxylate was prepared according to the procedure described for tert-butyl (S)-7-(benzyloxy)-1-(4-fluorophenyl)-3,4-dihydroisoquinoline-2(1H)-carboxylate (see COMPOUND 2115) after basic workup (saturated aqueous NaHCO₃/dichloromethane extraction). LCMS: RT=2.10 min., (M+H)⁺=440 (method A).

Synthesis of (S)—N-(azetidin-3-ylmethyl)-1-(4-fluorophenyl)-3,4-dihydroisoquinoline-2(1H)-carboxamide acetate (COMPOUND 2257).

Trifluoroacetic acid (0.20 ml, 2.6 mmol) was added to a solution of tert-butyl (S)-3-((1-(4-fluorophenyl)-1,2,3,4-tetrahydroisoquinoline-2-carboxamido)methyl)azetidine-1-carboxylate (100 mg, 0.228 mmol) in dichloromethane (1 mL). After stirring at room temperature for 30 minutes, the reaction mixture was evaporated under reduced pressure. The residue was dissolved in methanol (2 mL) and brought onto an SCX-2 column (2 g) and eluted with methanol until neutral. Next, the column was eluted with ammonia in methanol (2 M). The basic fraction was concentrated to dryness under reduced pressure. The residue was dissolved in dichloromethane (2 mL), trifluoroacetic acid (10 μL) was added and the mixture was concentrated under reduced pressure. The residue was lyophilized from water (3 mL) and purified by acidic preparative MPLC (Linear Gradient: t=0 min 5% A, t=1 min 5% A; t=16 min 50% A; t=17 min 100%; t=22 min 100% A; detection: 215/264 nm). The product containing fractions were combined and lyophilised to obtain the trifluoroacetic acid salt. This was partitioned between dichloromethane (3 mL) and saturated aqueous NaHCO₃ (3 mL). The layers were separated using a phase-separator and the organic filtrate was evaporated under reduced pressure to obtain the free base. The free base was dissolved in dichloromethane (2 mL), acetic acid (5 μL) was added, and the mixture was concentrated under reduced pressure. The residue was lyophilized from a mixture of acetonitrile and water (1:1, 4 mL) to give (S)—N-(azetidin-3-ylmethyl)-1-(4-fluorophenyl)-3,4-dihydroisoquinoline-2(1H-carboxamide acetate (COMPOUND 2257). LCMS: RT=1.02 min., (M+H)⁺=340 (method P).

### (S)—N—((R)-4,4-difluoro-1-methylpyrrolidin-3-yl)-1-(4-fluorophenyl)-3,4-dihydroisoquinoline-2(1H-carboxamide (Compound 2293) and (S)—N—((S)-4,4-difluoro-1-methylpyrrolidin-3-yl)-1-(4-fluorophenyl)-3,4-dihydroisoquinoline-2(1H-carboxamide (Compound 2294)

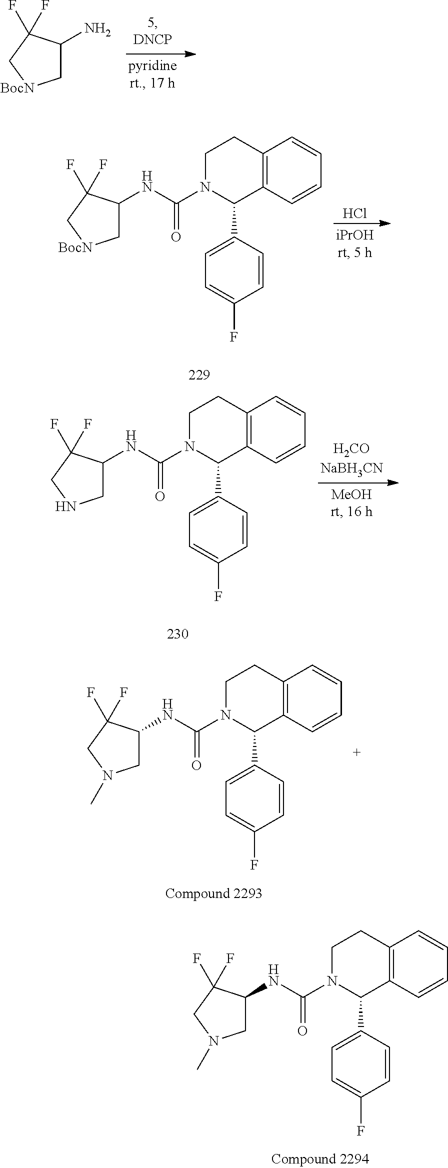

Synthesis of tert-butyl 3,3-difluoro-4-((S)-1-(4-fluorophenyl)-1,2,3,4-tetrahydroisoquinoline-2-carboxamido)pyrrolidine-1-carboxylate.

Starting from tert-butyl 4-amino-3,3-difluoropyrrolidine-1-carboxylate (251 mg, 1.127 mmol) and (S)-1-(4-fluorophenyl)-1,2,3,4-tetrahydroisoquinoline (250 mg, 1.100 mmol), tert-butyl 3,3-difluoro-4-((S)-1-(4-fluorophenyl)-1,2,3,4-tetrahydroisoquinoline-2-carboxamido)pyrrolidine-1-carboxylate was prepared as described for tert-butyl (S)-7-(benzyloxy)-1-(4-fluorophenyl)-3,4-dihydroisoquinoline-2(1H)-carboxylate (see COMPOUND 2115) and purified by flash column chromatography (silica, 0 to 50% ethyl acetate in heptane) after acidic (aqueous HCl (1 M)/dichloromethane) and basic (saturated aqueous NaHCO₃)/dichloromethane) workup. LCMS: RT=2.219 min., (M−H)⁻=474 (method A).

Synthesis of (1S)—N-(4,4-difluoropyrrolidin-3-yl)-1-(4-fluorophenyl)-3,4-dihydroisoquinoline-2(1H)-carboxamide (COMPOUND 2243+COMPOUND 2244).

Starting from tert-butyl 3,3-difluoro-4-((S)-1-(4-fluorophenyl)-1,2,3,4-tetrahydroisoquinoline-2-carboxamido)pyrrolidine-1-carboxylate (175 mg, 0.368 mmol), (1 S)—N-(4,4-difluoropyrrolidin-3-yl)-1-(4-fluorophenyl)-3,4-dihydroisoquinoline-2(1H)-carboxamide (COMPOUND 2243+COMPOUND 2244) was prepared as described for (S)—N-((trans)-3-amino-1-methylcyclobutyl)-1-(4-fluorophenyl)-3,4-dihydroisoquinoline-2(1H)-carboxamide (COMPOUND 2271) and purified by flash column chromatography (silica, 0 to 50% ethyl acetate in heptane). LCMS: 97%, RT=1.639 min., (M+H)⁺=376 (method A).

Synthesis of (S)—N—((R)-4,4-difluoro-1-methylpyrrolidin-3-yl)-1-(4-fluorophenyl)-3,4-dihydroisoquinoline-2(1H)-carboxamide (COMPOUND 2293) and (S)—N—((S)-4,4-difluoro-1-methylpyrrolidin-3-yl)-1-(4-fluorophenyl)-3,4-dihydroisoquinoline-2(1H)-carboxamide (COMPOUND 2294).

Formaldehyde (37 wt % solution in water, stabilized with 10-15% methanol, 66 μL, 0.879 mmol) was added to a solution of (1 S)—N-(4,4-difluoropyrrolidin-3-yl)-1-(4-fluorophenyl)-3,4-dihydroisoquinoline-2(1H)-carboxamide (COMPOUND 2243+COMPOUND 2244) in methanol (5 mL). After 30 minutes, sodium cyanoborohydride (33.1 mg, 0.527 mmol) was added and the reaction mixture was stirred at room temperature for 16 hours. The mixture was diluted with saturated aqueous NaHCO₃ (10 mL) and extracted with dichloromethane (2×10 mL). The combined organics were dried over Na₂SO₄ and evaporated under reduced pressure. The residue was purified by preparative SFC (method AE) to give (S)—N—((R)-4,4-difluoro-1-methylpyrrolidin-3-yl)-1-(4-fluorophenyl)-3,4-dihydroisoquinoline-2(1H-carboxamide (COMPOUND 2293) and (S)—N—((S)-4,4-difluoro-1-methylpyrrolidin-3-yl)-1-(4-fluorophenyl)-3,4-dihydroisoquinoline-2(1H-carboxamide (COMPOUND 2294) after lyophilization from a mixture of acetonitrile and water (1:1, 3 mL). COMPOUND 2293: LCMS: RT=1.125 min., (M+H)⁺=390 (method P). Chiral SFC: RT=2.088 min., (M+H)⁺=390 (method AD). COMPOUND 2294: LCMS: RT=1.123 min., (M+H)+=390 (method P). Chiral SFC: RT=2.368 min., (M+H)⁺=390 (method AD).

### (S)—N—((R)-4,4-difluoropyrrolidin-3-yl)-1-(4-fluorophenyl)-N-methyl-3,4-dihydroisoquinoline-2(1H-carboxamide (Compound 2296) and (S)—N—((S)-4,4-difluoropyrrolidin-3-yl)-1-(4-fluorophenyl)-N-methyl-3,4-dihydroisoquinoline-2(1H-carboxamide (Compound 2297)

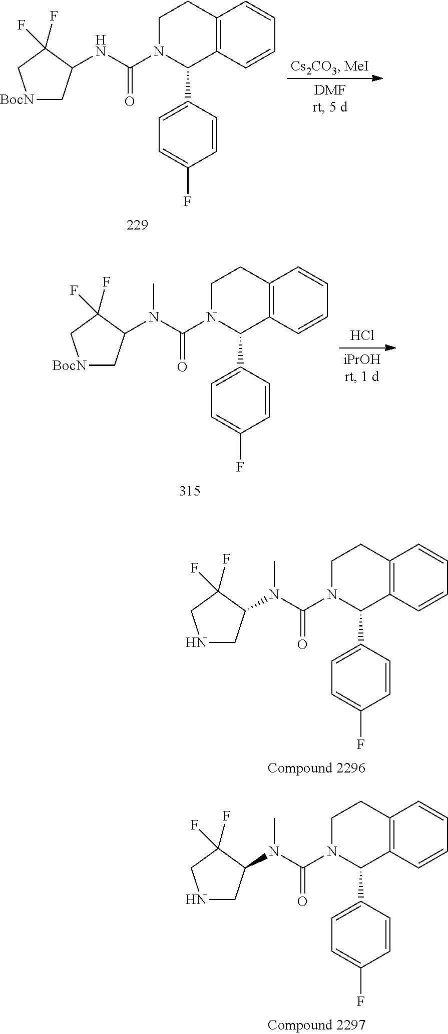

Synthesis of tert-butyl 3,3-difluoro-4-((S)-1-(4-fluorophenyl)-N-methyl-1,2,3,4-tetrahydroisoquinoline-2-carboxamido)pyrrolidine-1-carboxylate.

Methyl Iodide (201 μL, 3.23 mmol) was added to a mixture of tert-butyl 3,3-difluoro-4-((S)-1-(4-fluorophenyl)-1,2,3,4-tetrahydroisoquinoline-2-carboxamido)pyrrolidine-1-carboxylate (See COMPOUND 2243+COMPOUND 2244 and COMPOUND 2293+COMPOUND 2294, 279 mg, 0.406 mmol) and cesium carbonate (765 mg, 2.347 mmol) in N,N-dimethylformamide (5 mL). The reaction mixture was stirred at room temperature for 5 days. Then, the mixture was diluted with diethyl ether (50 mL) and the organic layer was washed with water (3×25 mL). The combined organic layers were dried over Na₂SO₄ and evaporated under reduced pressure. The residue was purified by flash column chromatography (silica, 0% to 75% ethyl acetate in heptane) to give tert-butyl 3,3-difluoro-4-((S)-1-(4-fluorophenyl)-N-methyl-1,2,3,4-tetrahydroisoquinoline-2-carboxamido)pyrrolidine-1-carboxylate. LCMS: RT=2.326 min., (M+Na)⁺=512 (method A).

(S)—N—((S)-4,4-difluoropyrrolidin-3-yl)-1-(4-fluorophenyl)-N-methyl-3,4-dihydroisoquinoline-2(1H)-carboxamide ( ) and (S)—N—((R)-4,4-difluoropyrrolidin-3-yl)-1-(4-fluorophenyl)-N-methyl-3,4-dihydroisoquinoline-2(1H)-carboxamide (COMPOUND 2296).

Starting from tert-butyl 3,3-difluoro-4-((S)-1-(4-fluorophenyl)-N-methyl-1,2,3,4-tetrahydroisoquinoline-2-carboxamido)pyrrolidine-1-carboxylate (171 mg (81 wt %), 0.283 mmol), (S)—N—((S)-4,4-difluoropyrrolidin-3-yl)-1-(4-fluorophenyl)-N-methyl-3,4-dihydroisoquinoline-2(1H-carboxamide (COMPOUND 2297 and (S)—N—((R)-4,4-difluoropyrrolidin-3-yl)-1-(4-fluorophenyl)-N-methyl-3,4-dihydroisoquinoline-2(1H-carboxamide (COMPOUND 2296) were prepared as described for (S)—N-((trans)-3-amino-1-methylcyclobutyl)-1-(4-fluorophenyl)-3,4-dihydroisoquinoline-2(1H-carboxamide (COMPOUND 2271). The molecules were purified by preparative chiral HPLC (method AF), and lyophilized from acetonitrile and water (1:1). COMPOUND 2296: LCMS: RT=1.147 min., (M+H)⁺=390 (method P). Chiral HPLC: RT=38.65 min. (method AG). COMPOUND 2297: LCMS (220 nm): RT=1.147 min., (M+H)⁺=390 (method P). Chiral HPLC: RT=27.02 min. (method AG).

### (S)—N—((R)-4,4-difluoro-1-methylpyrrolidin-3-yl)-1-(4-fluorophenyl)-N-methyl-3,4-dihydroisoquinoline-2(1H-carboxamide (Compound 2298) and (S)—N—((S)-4,4-difluoro-1-methylpyrrolidin-3-yl)-1-(4-fluorophenyl)-N-methyl-3,4-dihydroisoquinoline-2(1H-carboxamide (Compound 2299)

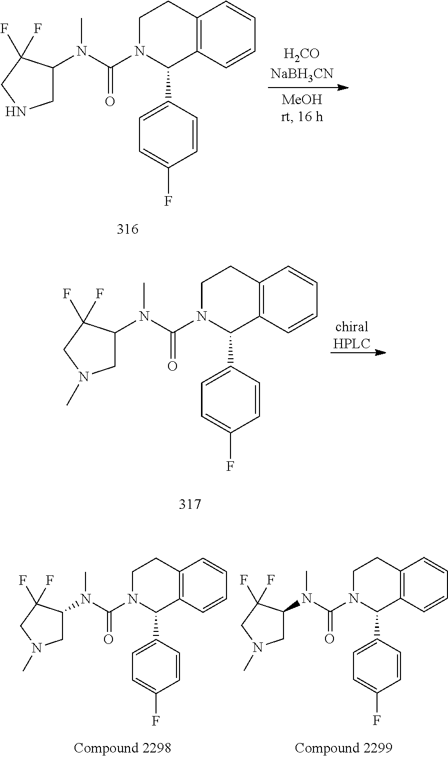

Synthesis of (S)—N—((R)-4,4-difluoro-1-methylpyrrolidin-3-yl)-1-(4-fluorophenyl)-N-methyl-3,4-dihydroisoquinoline-2(1H)-carboxamide (COMPOUND 2298) and (S)—N—((S)-4,4-difluoro-1-methylpyrrolidin-3-yl)-1-(4-fluorophenyl)-N-methyl-3,4-dihydroisoquinoline-2(1H)-carboxamide (COMPOUND 2299).

Formaldehyde (37 wt % solution in water, stabilized with 10-15% methanol, 55 μL, 0.738 mmol) was added to a solution of (1 S)—N-(4,4-difluoropyrrolidin-3-yl)-1-(4-fluorophenyl)-N-methyl-3,4-dihydroisoquinoline-2(1H)-carboxamide (See COMPOUND 2296+COMPOUND 2297, 115 mg, 0.295 mmol) in methanol (2 mL). After 30 minutes, sodium cyanoborohydride (27.8 mg, 0.443 mmol) was added and the reaction mixture was stirred at room temperature for 16 hours. The mixture was diluted with saturated aqueous NaHCO₃ (10 mL) and extracted with dichloromethane (2×10 mL). The combined organics were dried over Na₂SO₄ and evaporated under reduced pressure. The residue was purified by preparative chiral HPLC (method AF) to give (S)—N—((R)-4,4-difluoro-1-methylpyrrolidin-3-yl)-1-(4-fluorophenyl)-N-methyl-3,4-dihydroisoquinoline-2(1H-carboxamide (COMPOUND 2298) and ((S)—N—((S)-4,4-difluoro-1-methylpyrrolidin-3-yl)-1-(4-fluorophenyl)-N-methyl-3,4-dihydroisoquinoline-2(1H-carboxamide (COMPOUND 2299) after lyophilization from a mixture of acetonitrile and water (1:1, 3 mL). COMPOUND 2298: LCMS: RT=1.25 min., (M+H)⁺=404 (method P). Chiral HPLC: RT=11.27 min. (method AG). COMPOUND 2299: LCMS: RT=1.23 min., (M+H)⁺=404 (method P). Chiral HPLC: RT=15.85 min. (method AG).

Compounds 2303 and 2307 were prepared by a similar synthesis as Compound 2296. COMPOUND 2303 (M+H)⁺=384. RT=1.063 min., (method P)

COMPOUND 2307 (M+H)⁺=384. RT=1.060 min., (method P)

### ((S)-3-(aminomethyl)pyrrolidin-1-yl)((S)-1-(4-fluorophenyl)-3,4-dihydroisoquinolin-2(1 h-yl)methanone (compound 2279)

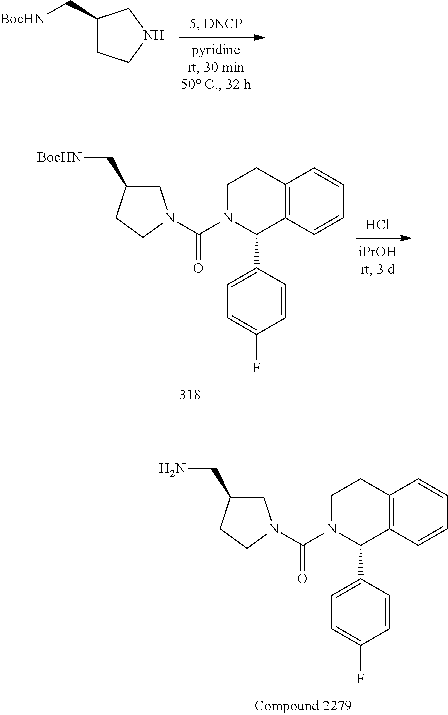 [SMILES: CC(C)(C)OC(=O)NC[C@@H]1CCNC1 ; CC(C)(C)OC(=O)NC[C@@H]1CCN(C(=O)N2CCc3ccccc3[C@@H]2c2ccc(F)cc2)C1 ; Cl ; NC[C@@H]1CCN(C(=O)N2CCc3ccccc3[C@@H]2c2ccc(F)cc2)C1]

Synthesis of (((S)-1-((S)-1-(4-fluorophenyl)-1,2,3,4-tetrahydroisoquinoline-2-carbonyl)pyrrolidin-3-yl)methyl)carbamate.

Starting from (S)-1-(4-fluorophenyl)-1,2,3,4-tetrahydroisoquinoline (175 mg, 0.77 mmol) and tert-butyl N-((3R)-pyrrolidin-3-ylmethyl)carbamate (170 mg, 0.85 mmol; and 77 mg, 0.39 mmol after 16 hours at 50° C.), (((S)-1-((S)-1-(4-fluorophenyl)-1,2,3,4-tetrahydroisoquinoline-2-carbonyl)pyrrolidin-3-yl)methyl)carbamate was prepared as described for tert-butyl (S)-7-(benzyloxy)-1-(4-fluorophenyl)-3,4-dihydroisoquinoline-2(1H)-carboxylate (see COMPOUND 2115). The molecule was purified by flash column chromatography (silica, 25% to 100% ethyl acetate in heptane) after acidic (aqueous HCl (1 M)/dichloromethane) and basic (saturated aqueous NaHCO₃/dichloromethane) workup. LCMS: RT=2.179 min., (M+H)⁺=454 (method A).

Synthesis of ((S)-3-(aminomethyl)pyrrolidin-1-yl)((S)-1-(4-fluorophenyl)-3,4-dihydroisoquinolin-2(1H)-yl)methanone (COMPOUND 2279).

Starting from tert-butyl (((S)-1-((S)-1-(4-fluorophenyl)-1,2,3,4-tetrahydroisoquinoline-2-carbonyl)pyrrolidin-3-yl)methyl)carbamate (289 mg, 0.637 mmol), ((S)-3-(aminomethyl)pyrrolidin-1-yl)((S)-1-(4-fluorophenyl)-3,4-dihydroisoquinolin-2(1)-yl)methanone (COMPOUND 2279) was prepared as described for (S)—N-((trans)-3-amino-1-methylcyclobutyl)-1-(4-fluorophenyl)-3,4-dihydroisoquinoline-2(1H-carboxamide (COMPOUND 2271), purified by acidic preparative MPLC (Linear Gradient: t=0 min 5% A, t=1 min 5% A; t=16 min 50% A; t=17 min 100%; t=22 min 100% A; detection: 220/254 nm). The product was isolated by dichloromethane extraction from basified (aqueous saturated NaHCO₃) fractions and lyophilization from a mixture of acetonitrile and water (1:1). LCMS: RT=1.048 min., (M+H)⁺=354 (method P).

Compound 2288 was prepared by a similar synthesis as for Compound 2279. COMPOUND 2288 (M+H)⁺=354. RT=1.045 min., (method P)

### ((S)-1-(4-fluorophenyl)-3,4-dihydroisoquinolin-2(1H)-yl)((R)-6-oxa-2,9-diazaspiro[4.5]decan-2-yl)methanone (Compound 2323); ((S)-1-(4-fluorophenyl)-3,4-dihydroisoquinolin-2(1H)-yl)((R)-6-oxa-2,9-diazaspiro[4.5]decan-2-yl)methanone (Compound 2324); ((R)-1-(4-fluorophenyl)-3,4-dihydroisoquinolin-2(1H)-yl)((S)-6-oxa-2,9-diazaspiro[4.5]decan-2-yl)methanone (Compound 2325); ((S)-1-(4-fluorophenyl)-3,4-dihydroisoquinolin-2(1)-yl)((S)-6-oxa-2,9-diazaspiro[4.5]decan-2-yl)methanone (Compound 2326)

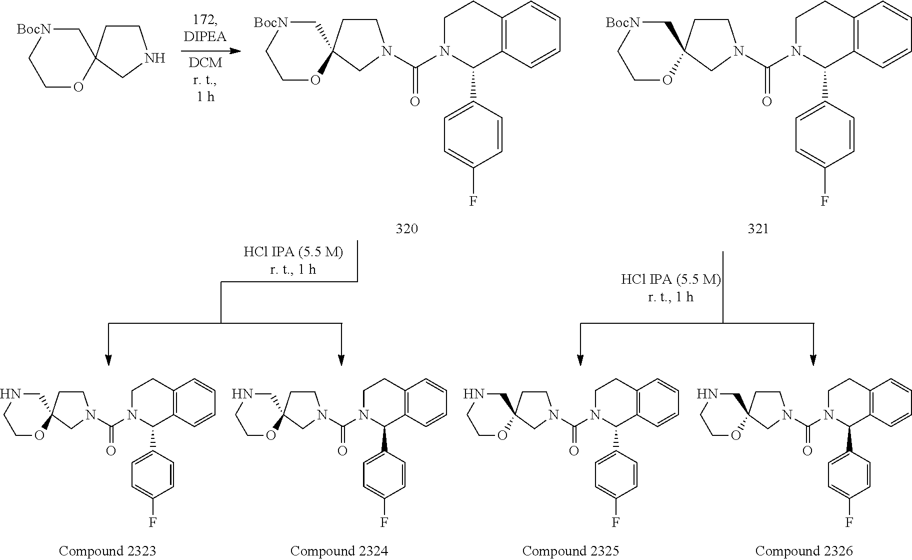

Synthesis of (S)-1-(4-fluorophenyl)-3,4-dihydroisoquinoline-2(1H)-carbonyl chloride.

A solution of (S)-1-(4-fluorophenyl)-1,2,3,4-tetrahydroisoquinoline (20.0 g, 88.0 mmol) and pyridine (7.81 mL, 96.8 mmol) in toluene (dry, 80 ml) was added dropwise to a stirring solution of triphosgene (9.92 g, 33.4 mmol) in toluene (dry, 60 ml). Then the mixture was warmed to 80° C. for 3 hours and allowed to cool to room temperature. The mixture was filtered over celite and washed with diethyl ether (150 mL). The combined filtrates were filtered over celite a second time and the filtrate was evaporated under reduced pressure. The residue was taken up in dichloromethane, filtered over a short path of silica, and washed with dichloromethane. The filtrate was evaporated under reduced pressure. The residue was triturated with heptane (50 mL) while stirring and cooled in an ice/water bath. After 1 hour, the formed solids were collected by filtration, washed with ice-cold pentane (2×25 mL), and dried under reduced pressure at 30° C. to give (S)-1-(4-fluorophenyl)-3,4-dihydroisoquinoline-2(1H-carbonyl chloride. LCMS: RT=2.26 min., (M+H)⁺=290/292 (Cl-pattern) (method B).

Synthesis of tert-butyl (S)-2-((S)-1-(4-fluorophenyl)-1,2,3,4-tetrahydroisoquinoline-2-carbonyl)-6-oxa-2,9-diazaspiro[4.5]decane-9-carboxylate and tert-butyl (R)-2-((S)-1-(4-fluorophenyl)-1,2,3,4-tetrahydroisoquinoline-2-carbonyl)-6-oxa-2,9-diazaspiro[4.5]decane-9-carboxylate.

N,N-diisopropylethylamine (360 μL, 2.06 mmol) was added to a solution of tert-butyl 6-oxa-2,9-diazaspiro[4.5]decane-9-carboxylate (250 mg, 1.03 mmol) and (S)-1-(4-fluorophenyl)-3,4-dihydroisoquinoline-2(1H-carbonyl chloride (269 mg, 0.929 mmol) in dichloromethane (3.0 mL). After stirring for 1 hour, the mixture was diluted with dichloromethane (15 mL), and washed with aqueous HCl (1 M, 3×10 mL) and brine (10 mL). The organic layer was dried over Na₂SO₄ and evaporated under reduced pressure. The residue was purified by preparative SFC (method S) to give tert-butyl (S)-2-((S)-1-(4-fluorophenyl)-1,2,3,4-tetrahydroisoquinoline-2-carbonyl)-6-oxa-2,9-diazaspiro[4.5]decane-9-carboxylate as the first eluting isomer and tert-butyl (R)-2-((S)-1-(4-fluorophenyl)-1,2,3,4-tetrahydroisoquinoline-2-carbonyl)-6-oxa-2,9-diazaspiro[4.5]decane-9-carboxylate as the second eluting isomer. The absolute configuration of the of the spirocyclic center was arbitrarily assigned. First eluting isomer: LCMS: RT=1.95 min., (M+H)⁺=496 (method P). SFC: RT=3.65 min., (M+H)⁺=496 (method F).

Second eluting isomer: LCMS: RT=1.96 min., (M+H)⁺=496 (method P). SFC: RT=5.68 min., (M+H)⁺=496 (method F).

Synthesis of ((R)-1-(4-fluorophenyl)-3,4-dihydroisoquinolin-2(1H)-yl)((R)-6-oxa-2,9-diazaspiro[4.5]decan-2-yl)methanone (COMPOUND 2323) and ((S)-1-(4-fluorophenyl)-3,4-dihydroisoquinolin-2(1H)-yl)((R)-6-oxa-2,9-diazaspiro[4.5]decan-2-yl)methanone (COMPOUND 2324).

Starting from tert-butyl (S)-2-((S)-1-(4-fluorophenyl)-1,2,3,4-tetrahydroisoquinoline-2-carbonyl)-6-oxa-2,9-diazaspiro[4.5]decane-9-carboxylate (209 mg, 0.422 mmol), ((R)-1-(4-fluorophenyl)-3,4-dihydroisoquinolin-2(1H-yl)((R)-6-oxa-2,9-diazaspiro[4.5]decan-2-yl)methanone (COMPOUND 2323) as the first eluting isomer and ((S)-1-(4-fluorophenyl)-3,4-dihydroisoquinolin-2(1H-yl)((R)-6-oxa-2,9-diazaspiro[4.5]decan-2-yl)methanone (COMPOUND 2324) as the second eluting isomer were prepared according to the procedure described for (S)—N-((trans)-3-amino-1-methylcyclobutyl)-1-(4-fluorophenyl)-3,4-dihydroisoquinoline-2(1H-carboxamide (COMPOUND 2271). A solution of the product was eluted through an SCX-2 column and purified by preparative SFC (method AM). Through monitoring the epimerization of the tetrahydroisoquinoline stereocenter by LCMS, the absolute configuration of that chiral center for each isomer could be assigned based on the (S) configured starting material. First eluting isomer: LCMS: RT=2.69 min., (M+H)⁺=396 (method AK). SFC: RT=3.97 min., (M+H)⁺=396 (method AD). Second eluting isomer: LCMS: RT=2.65 min., (M+H)⁺=396 (method AK). SFC: RT=4.24 min., (M+H)⁺=396 (method AD).

Synthesis of ((R)-1-(4-fluorophenyl)-3,4-dihydroisoquinolin-2(1H)-yl)((S)-6-oxa-2,9-diazaspiro[4.5]decan-2-yl)methanone (COMPOUND 2325) and ((S)-1-(4-fluorophenyl)-3,4-dihydroisoquinolin-2(1H)-yl)((S)-6-oxa-2,9-diazaspiro[4.5]decan-2-yl)methanone (COMPOUND 2326).

Starting from tert-butyl (R)-2-((S)-1-(4-fluorophenyl)-1,2,3,4-tetrahydroisoquinoline-2-carbonyl)-6-oxa-2,9-diazaspiro[4.5]decane-9-carboxylate (0.19 g, 0.39 mmol), ((R)-1-(4-fluorophenyl)-3,4-dihydroisoquinolin-2(1)-yl)((S)-6-oxa-2,9-diazaspiro[4.5]decan-2-yl)methanone (COMPOUND 2325) the first eluting isomer and ((S)-1-(4-fluorophenyl)-3,4-dihydroisoquinolin-2(1)-yl)((S)-6-oxa-2,9-diazaspiro[4.5]decan-2-yl)methanone (COMPOUND 2326) as the second eluting isomer were prepared according to the procedure described for (S)—N-((trans)-3-amino-1-methylcyclobutyl)-1-(4-fluorophenyl)-3,4-dihydroisoquinoline-2(1H-carboxamide (COMPOUND 2271). A solution of the product was eluted through an SCX-2 column, and purified by acidic preparative MPLC (Linear Gradient: t=0 min 2% A, t=1 min 2% A; t=14 min 25% A; t=16 min 25% A; t=18 min 30% A; t=19 min 100% A; detection 220/254/280 nm) and additional purification of the first eluting isomer by preparative SFC (method AN). Through monitoring the epimerization of the tetrahydroisoquinoline stereocenter by LCMS, the absolute configuration of that chiral center for each isomer could be assigned based on the (S) configured starting material. COMPOUND 2325: LCMS: RT=1.07 min., (M+H)⁺=396 (method P). SFC: RT=4.29 min., (M+H)⁺=396 (method AD). COMPOUND 2326: LCMS: RT=2.69 min., (M+H)⁺=396 (method AK). SFC: RT=4.45 min., (M+H)⁺=396 (method AD).

### ((S)-3-((dimethylamino)methyl)pyrrolidin-1-yl)((S)-1-(4-fluorophenyl)-3,4-dihydroisoquinolin-2(1H)-yl)methanone (Compound 2313)

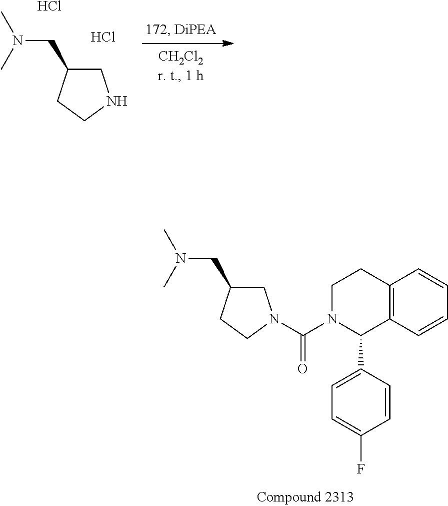

Synthesis of ((S)-3-((dimethylamino)methyl)pyrrolidin-1-yl)((S)-1-(4-fluorophenyl)-3,4-dihydroisoquinolin-2(1H)-yl)methanone (COMPOUND 2313).

Starting from (R)—N,N-dimethyl-1-(pyrrolidin-3-yl)methanamine dihydrochloride (69.4 mg, 0.345 mmol) and N,N-diisopropylethylamine (0.181 mL, 1.035 mmol) and (S)-1-(4-fluorophenyl)-3,4-dihydroisoquinoline-2(1H-carbonyl chloride (see COMPOUND 2324, 100 mg, 0.345 mmol), ((S)-3-((dimethylamino)methyl)pyrrolidin-1-yl)((S)-1-(4-fluorophenyl)-3,4-dihydroisoquinolin-2(1H-yl)methanone (COMPOUND 2313) was prepared as described for tert-butyl (S and R)-2-((S)-1-(4-fluorophenyl)-1,2,3,4-tetrahydroisoquinoline-2-carbonyl)-6-oxa-2,9-diazaspiro[4.5]decane-9-carboxylate (see COMPOUND 2324). Following basic workup (saturated aqueous NaHCO₃/dichloromethane), purification by acidic preparative MPLC (Linear gradient: t=0 min 5% A, t=1 min 5%; t=1 min 10% A; t=15 min 50% A; t=1 min 100%; t=5 min 100% A; detection: 220/263 nm) was followed by desalting (SCX-2 (5 g) ion exchange chromatography), and lyophilization from acetonitrile and water (1:1). LCMS: RT=2.71 min., (M+H)⁺=382 (Method AK).

Compound 2314 was prepared by a similar synthesis as Compound 2313. COMPOUND 2314 (M+H)⁺=382. RT=2.73 min., (Method AK)

### (S)-1-(4-fluorophenyl)-N-((cis-4-hydroxypyrrolidin-3-yl)methyl)-3,4-dihydroisoquinoline-2(1H)-carboxamide (Compound 2308)

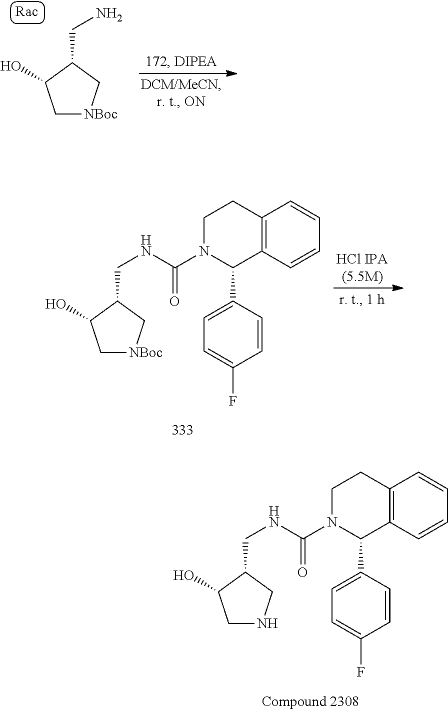 [SMILES: CC(C)(C)OC(=O)N1C[C@@H](CN)[C@@H](O)C1 ; CC(C)(C)OC(=O)N1C[C@@H](CNC(=O)N2CCc3ccccc3[C@@H]2c2ccc(F)cc2)[C@@H](O)C1 ; O=C(NC[C@@H]1CNC[C@@H]1O)N1CCc2ccccc2[C@@H]1c1ccc(F)cc1]

Synthesis of tert-butyl cis-3-(((S)-1-(4-fluorophenyl)-1,2,3,4-tetrahydroisoquinoline-2-carboxamido)methyl)-4-hydroxypyrrolidine-1-carboxylate.

Starting from tert-butyl cis-3-(aminomethyl)-4-hydroxypyrrolidine-1-carboxylate (70 mg, 0.32 mmol) and (S)-1-(4-fluorophenyl)-3,4-dihydroisoquinoline-2(1H-carbonyl chloride (see COMPOUND 2324), tert-butyl cis-3-(((S)-1-(4-fluorophenyl)-1,2,3,4-tetrahydroisoquinoline-2-carboxamido)methyl)-4-hydroxypyrrolidine-1-carboxylate (0.152 g (90 wt %)) was prepared according to the procedure described for tert-butyl (S and R)-2-((S)-1-(4-fluorophenyl)-1,2,3,4-tetrahydroisoquinoline-2-carbonyl)-6-oxa-2,9-diazaspiro[4.5]decane-9-carboxylate (see COMPOUND 2324; 3:2 mixture of dichloromethane and acetonitrile) and used without additional purification. LCMS: RT=1.76 min., (M−tBu+H)⁺=414 (method P).

Synthesis of (S)-1-(4-fluorophenyl)-N-((cis-4-hydroxypyrrolidin-3-yl)methyl)-3,4-dihydroisoquinoline-2(1H)-carboxamide (COMPOUND 2308).

Starting from tert-butyl cis-3-(((S)-1-(4-fluorophenyl)-1,2,3,4-tetrahydroisoquinoline-2-carboxamido)methyl)-4-hydroxypyrrolidine-1-carboxylate (69 mg, 0.15 mmol), (S)-1-(4-fluorophenyl)-N-((cis-4-hydroxypyrrolidin-3-yl)methyl)-3,4-dihydroisoquinoline-2(1H-carboxamide (COMPOUND 2308) was prepared according to the procedure described for (S)—N-((trans)-3-amino-1-methylcyclobutyl)-1-(4-fluorophenyl)-3,4-dihydroisoquinoline-2(1H-carboxamide (COMPOUND 2271). Post workup, a solution of the product was eluted through an SCX-2 column (1 g), and lyophilized from a mixture of acetonitrile and water (1:1). LCMS: RT=1.03 min., (M+H)⁺=370 (method P).

### (S)-1-(4-fluorophenyl)-N-((3S,4S)-4-fluoropyrrolidin-3-yl)-3,4-dihydroisoquinoline-2(1H-carboxamide (Compound 2351)

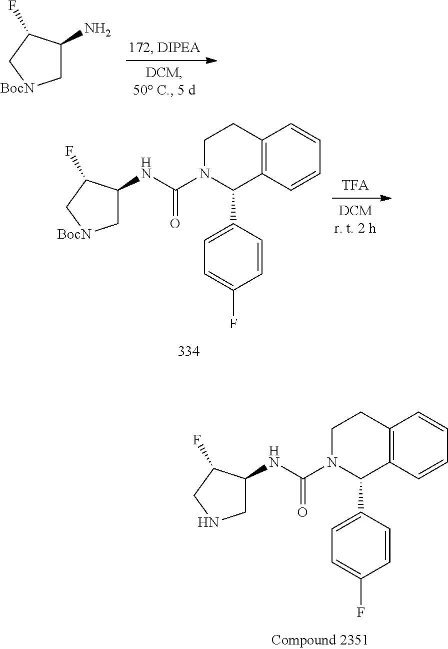 [SMILES: CC(C)(C)OC(=O)N1C[C@H](N)[C@@H](F)C1 ; CC(C)(C)OC(=O)N1C[C@H](NC(=O)N2CCc3ccccc3[C@@H]2c2ccc(F)cc2)[C@@H](F)C1 ; O=C(N[C@H]1CNC[C@@H]1F)N1CCc2ccccc2[C@@H]1c1ccc(F)cc1]

Synthesis of tert-butyl (3S,4S)-3-fluoro-4-((S)-1-(4-fluorophenyl)-1,2,3,4-tetrahydroisoquinoline-2-carboxamido)pyrrolidine-1-carboxylate.

(S)-1-(4-fluorophenyl)-3,4-dihydroisoquinoline-2(1H-carbonyl chloride (see COMPOUND 2324, 70.9 mg, 0.245 mmol) was added to a solution of tert-butyl (3S,4S)-3-amino-4-fluoropyrrolidine-1-carboxylate (50 mg, 0.245 mmol) and N,N-diisopropylethylamine (0.047 mL, 0.269 mmol) in dichloromethane (2 mL). After stirring for 5 days at 50° C., the reaction mixture was mixture was concentrated to dryness under reduced pressure. The residue was purified by flash column chromatography (silica, 0 to 50% ethyl acetate in heptane) to give tert-butyl (3S,4S)-3-fluoro-4-((S)-1-(4-fluorophenyl)-1,2,3,4-tetrahydroisoquinoline-2-carboxamido)pyrrolidine-1-carboxylate. LCMS: RT=2.19 min., (M−H)⁺=456 (method B).

Synthesis of (S)-1-(4-fluorophenyl)-N-((3S,4S)-4-fluoropyrrolidin-3-yl)-3,4-dihydroisoquinoline-2(1H)-carboxamide (COMPOUND 2351).

Starting from tert-butyl (3S,4S)-3-fluoro-4-((S)-1-(4-fluorophenyl)-1,2,3,4-tetrahydroisoquinoline-2-carboxamido)pyrrolidine-1-carboxylate (79 mg, 0.173 mmol), give (S)-1-(4-fluorophenyl)-N-((3S,4S)-4-fluoropyrrolidin-3-yl)-3,4-dihydroisoquinoline-2(1)-carboxamide (COMPOUND 2351) was prepared as described for (S)-1-(4-fluorophenyl)-N-(3-(prop-2-yn-1-ylamino)bicyclo[1.1.1]pentan-1-yl)-3,4-dihydroisoquinoline-2(1H-carboxamide 2,2,2-trifluoroacetate (COMPOUND 2201). Post workup, the material was desalted (SCX-2 (2 g) ion exchange chromatography). LCMS: RT=1.03 min., (M+H)⁺=358 (method P). SFC: RT=2.80 min., (M+H)⁺=358 (method AD).

Compounds 2352, 2253, and 2360 were prepared by a similar synthesis as Compound 2351. COMPOUND 2352 (M+H)⁺=358. RT=1.04 min., (method P)

COMPOUND 2353 (M+H)⁺=358. RT=1.04 min., (method P)

COMPOUND 2360 (M+H)⁺=358. RT=1.04 min., (method P)

### ((R)-2-(aminomethyl)morpholino)((S)-1-(4-fluorophenyl)-3,4-dihydroisoquinolin-2(1H-yl)methanone (Compound 2302)

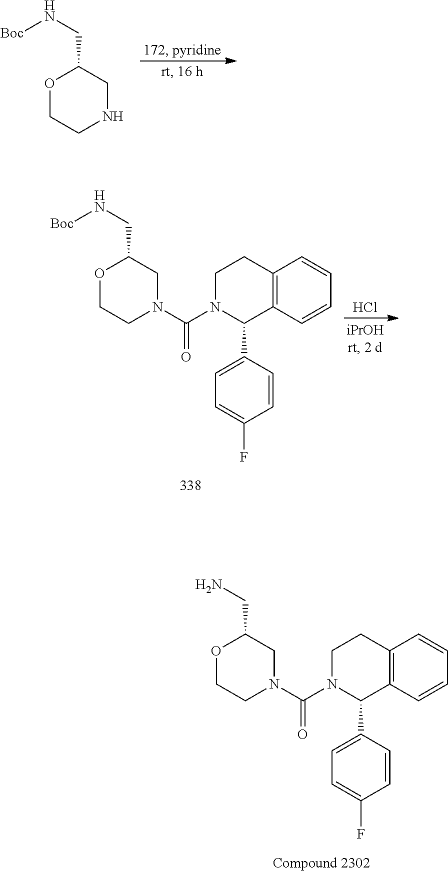 [SMILES: CC(C)(C)OC(=O)NC[C@@H]1CNCCO1 ; CC(C)(C)OC(=O)NC[C@@H]1CN(C(=O)N2CCc3ccccc3[C@@H]2c2ccc(F)cc2)CCO1 ; Cl ; NC[C@@H]1CN(C(=O)N2CCc3ccccc3[C@@H]2c2ccc(F)cc2)CCO1]

Synthesis of tert-butyl (((R)-4-((S)-1-(4-fluorophenyl)-1,2,3,4-tetrahydroisoquinoline-2-carbonyl)morpholin-2-yl)methyl)carbamate.

Tert-butyl (S)-(morpholin-2-ylmethyl)carbamate (226 mg, 1.044 mmol) was added to a solution of (S)-1-(4-fluorophenyl)-3,4-dihydroisoquinoline-2(1H-carbonyl chloride (see COMPOUND 2324, 275 mg, 0.949 mmol) in pyridine (1.5 mL). The reaction mixture was stirred for 16 hours at room temperature. The reaction mixture was diluted with dichloromethane (25 mL), washed with aqueous HCl (1 M, 25 mL), saturated aqueous NaHCO₃ (25 mL), and brine (25 mL), dried over Na₂SO₄, and evaporated under reduced pressure to give tert-butyl (((R)-4-((S)-1-(4-fluorophenyl)-1,2,3,4-tetrahydroisoquinoline-2-carbonyl)morpholin-2-yl)methyl)carbamate. LCMS: RT=2.166 min., (M+H)⁺=470 (method A).

Synthesis of ((R)-2-(aminomethyl)morpholino)((S)-1-(4-fluorophenyl)-3,4-dihydroisoquinolin-2(1H)-yl)methanone (COMPOUND 2302).

Starting from tert-butyl (((R)-4-((S)-1-(4-fluorophenyl)-1,2,3,4-tetrahydroisoquinoline-2-carbonyl)morpholin-2-yl)methyl)carbamate (150 mg, 0.319 mmol), ((R)-2-(aminomethyl)morpholino)((S)-1-(4-fluorophenyl)-3,4-dihydroisoquinolin-2(1H-yl)methanone (COMPOUND 2302) was prepared as described for (S)—N-((trans)-3-amino-1-methylcyclobutyl)-1-(4-fluorophenyl)-3,4-dihydroisoquinoline-2(1H-carboxamide (COMPOUND 2271). A solution of the product was eluted through an SCX-2 column (5 g) and lyophilized from a mixture of acetonitrile and water (1:1). LCMS: RT=1.052 min., (M+H)⁺=370 (method P).

Compound 2303 was prepared by a similar synthesis as Compound 2302. COMPOUND 2303 (M+H)⁺=370. RT=1.05 min., (method P)

### ((S)-1-(4-fluorophenyl)-3,4-dihydroisoquinolin-2(1H)-yl)((R)-2-((methylamino)methyl)morpholino)methanone (Compound 2306)

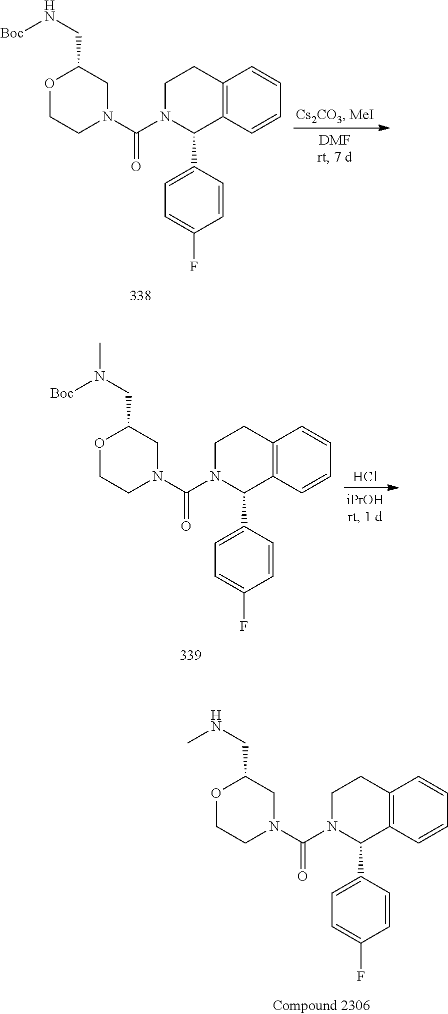

Synthesis of tert-butyl (((S)-4-((S)-1-(4-fluorophenyl)-1,2,3,4-tetrahydroisoquinoline-2-carbonyl)morpholin-2-yl)methyl)(methyl)carbamate.

Methyliodide (128 μL, 2.058 mmol) was added to a mixture of tert-butyl (((R)-4-((S)-1-(4-fluorophenyl)-1,2,3,4-tetrahydroisoquinoline-2-carbonyl)morpholin-2-yl)methyl)carbamate (see COMPOUND 2302, 249 mg, 0.514 mmol) and cesium carbonate (838 mg, 2.572 mmol) in N,N-dimethylformamide (2.5 mL) and stirred for 7 days. The mixture was diluted with diethyl ether (25 mL), washed with water (3×25 mL), dried over Na₂SO₄, and evaporated under reduced pressure. The residue was purified by flash column chromatography (silica, 0% to 75% ethyl acetate in heptane) to give tert-butyl (((S)-4-((S)-1-(4-fluorophenyl)-1,2,3,4-tetrahydroisoquinoline-2-carbonyl)morpholin-2-yl)methyl)(methyl)carbamate. LCMS: RT=2.238 min., (M+H)⁺=484 (method A).

Synthesis of ((S)-1-(4-fluorophenyl)-3,4-dihydroisoquinolin-2(1H)-yl)((R)-2-((methylamino)methyl)morpholino)methanone (COMPOUND 2306).

Starting from tert-butyl (((S)-4-((S)-1-(4-fluorophenyl)-1,2,3,4-tetrahydroisoquinoline-2-carbonyl)morpholin-2-yl)methyl)(methyl)carbamate (112 mg (90 wt %), 0.209 mmol), ((S)-1-(4-fluorophenyl)-3,4-dihydroisoquinolin-2(1H-yl)((R)-2-((methylamino)methyl)morpholino)methanone (COMPOUND 2306) was prepared as described for (S)—N-((trans)-3-amino-1-methylcyclobutyl)-1-(4-fluorophenyl)-3,4-dihydroisoquinoline-2(1H-carboxamide (COMPOUND 2271). A solution of the product was eluted through an SCX-2 column (1 g) and lyophilized from a mixture of acetonitrile and water (1:1). LCMS: RT=1.063 min., (M+H)⁺=384 (method P).

### Azetidin-3-ylmethyl (S)-1-(4-fluorophenyl)-3,4-dihydroisoquinoline-2(1H)-carboxylate 2,2,2-trifluoroacetate (Compound 2246)

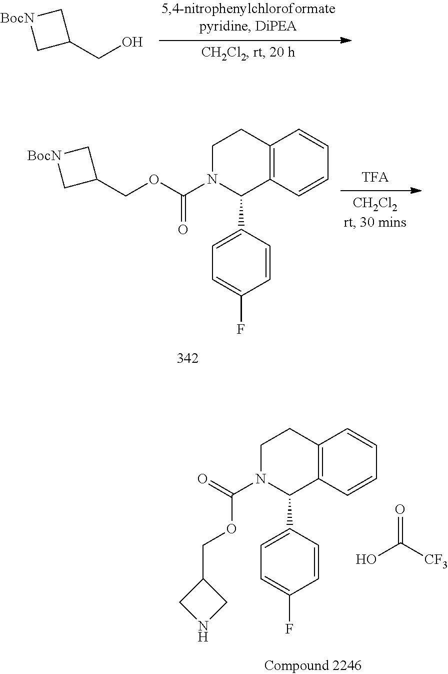

Synthesis of (1-(tert-butoxycarbonyl)azetidin-3-yl)methyl (S)-1-(4-fluorophenyl)-3,4-dihydroisoquinoline-2(1H)-carboxylate.

Starting from tert-butyl 3-(hydroxymethyl)azetidine-1-carboxylate (60 mg, 0.32 mmol) and (S)-1-(4-fluorophenyl)-1,2,3,4-tetrahydroisoquinoline (73 mg, 0.32 mmol), (1-(tert-butoxycarbonyl)azetidin-3-yl)methyl (S)-1-(4-fluorophenyl)-3,4-dihydroisoquinoline-2(1H-carboxylate (71 mg) was prepared according to the procedure described for tert-butyl (cis-3-((S)-1-(4-fluorophenyl)-1,2,3,4-tetrahydroisoquinoline-2-carboxamido)cyclobutyl)carbamate (see COMPOUND 2236) after purification by flash column chromatography (silica, 10 to 100% ethyl acetate in heptane). LCMS: RT=2.24 min., (M+Na)⁺=463 (method A).

Synthesis of azetidin-3-ylmethyl (S)-1-(4-fluorophenyl)-3,4-dihydroisoquinoline-2(1H)-carboxylate 2,2,2-trifluoroacetate (COMPOUND 2246).

Starting from (1-(tert-butoxycarbonyl)azetidin-3-yl)methyl (S)-1-(4-fluorophenyl)-3,4-dihydroisoquinoline-2(1H-carboxylate (71 mg, 0.16 mmol), azetidin-3-ylmethyl (S)-1-(4-fluorophenyl)-3,4-dihydroisoquinoline-2(1H)-carboxylate 2,2,2-trifluoroacetate (COMPOUND 2246) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H-carboxamide 2,2,2-trifluoroacetate (COMPOUND 2201). After desalting (SCX-2 (1 g) ion exchange chromatography) the molecule was purified by acidic preparative MPLC (Linear Gradient: t=0 min 5% A, t=1 min 5% A; t=16 min 50% A; t=17 min 100%; t=22 min 100% A; detection: 220 nm), concentrated from a mixture of dichloromethane (0.5 mL) and trifluoroacetic acid (10 μL), and lyophilized from a mixture of acetonitrile and water (1:1, 4 mL). LCMS: RT=2.63 min., (M+H)⁺=341 (method AK).

Compounds 2258, 2260, 2263, 2272 were prepared by a similar synthesis as Compound 2246:

COMPOUND 2258 (M+H)⁺=359. RT=1.12 min., (method P)

COMPOUND 2260 (M+H)⁺=371. RT=2.63 min., (method AK)

COMPOUND 2263 (M+H)⁺=371. RT=2.703 min., (method AK)

COMPOUND 2272 (M+H)⁺=341. RT=1.07 min., (method P) (R)-1-methylpyrrolidin-3-yl (S)-1-(4-fluorophenyl)-3,4-dihydroisoquinoline-2(1H)-carboxylate hydrochloride (COMPOUND 2250)

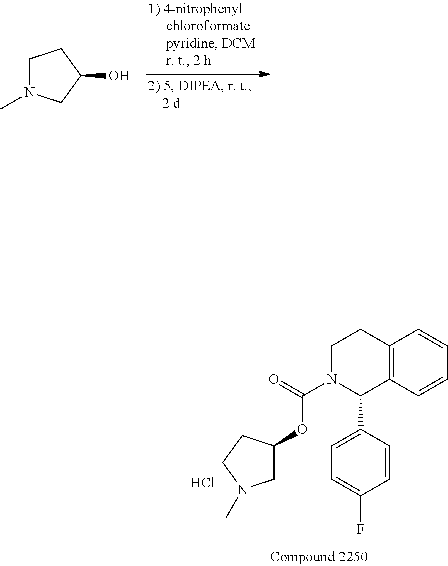

Synthesis of (R)-1-methylpyrrolidin-3-yl (S)-1-(4-fluorophenyl)-3,4-dihydroisoquinoline-2(1H)-carboxylate hydrochloride (COMPOUND 2250).

Starting from (R)-1-methylpyrrolidin-3-ol (109 μL, 0.989 mmol) and (S)-1-(4-fluorophenyl)-1,2,3,4-tetrahydroisoquinoline (270 mg, 1.19 mmol), (R)-1-methylpyrrolidin-3-yl (S)-1-(4-fluorophenyl)-3,4-dihydroisoquinoline-2(1H-carboxylate hydrochloride (COMPOUND 2250) was prepared according to the procedure described for tert-butyl (cis-3-((S)-1-(4-fluorophenyl)-1,2,3,4-tetrahydroisoquinoline-2-carboxamido)cyclobutyl)carbamate (see COMPOUND 2236). After a basic workup, the molecule was purified by basic preparative MPLC (Linear Gradient: t=0 min 5% A, t=1 min 5% A; t=2 min 40% A; t=17 min 60% A; t=18 min 100% A; t=23 min 100% A; detection 220/264 nm) and acidic preparative MPLC (Linear Gradient: t=0 min 5% A, t=1 min 5% A; t=17 min 40% A; t=18 min 100% A; t=23 min 100% A; detection 220/270 nm). The residue was lyophilized from acetonitrile (0.5 mL) and aqueous HCl (0.5 M, 1 mL). LCMS: RT=1.10 min., (M+H)⁺=355 (method P).

Compound 2251 was prepared by a similar synthesis as Compound 2250.

COMPOUND 2251 (M+H)⁺=355. RT=1.09 min., (method P)

(R)-pyrrolidin-3-yl (R)-1-(4-fluorophenyl)-3,4-dihydroisoquinoline-2(1H-carboxylate (COMPOUND 2266)

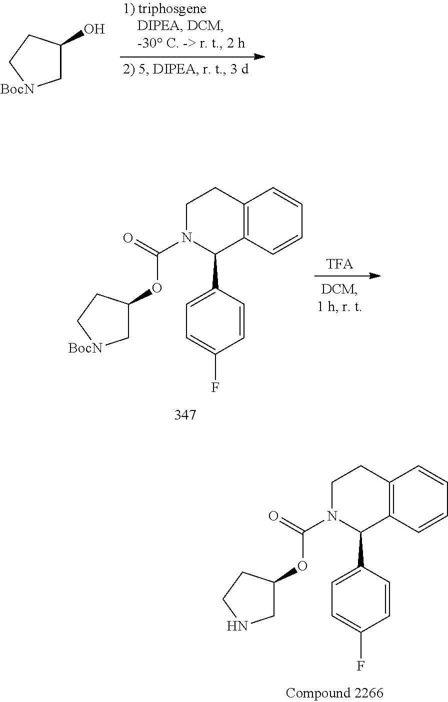 [SMILES: CC(C)(C)OC(=O)N1CC[C@@H](O)C1 ; CC(C)(C)OC(=O)N1CC[C@@H](OC(=O)N2CCc3ccccc3[C@H]2c2ccc(F)cc2)C1 ; O=C(O[C@@H]1CCNC1)N1CCc2ccccc2[C@H]1c1ccc(F)cc1]

Synthesis of (R)-1-(tert-butoxycarbonyl)pyrrolidin-3-yl (R)-1-(4-fluorophenyl)-3,4-dihydroisoquinoline-2(1H)-carboxylate.

Under an argon atmosphere, a solution of tert-butyl (R)-3-hydroxypyrrolidine-1-carboxylate (0.20 g, 1.1 mmol) in dichloromethane (5.0 mL) was added to a solution of bis(trichloromethyl) carbonate (0.11 g, 0.36 mmol) in dichloromethane (1.0 mL) at −30° C. Then, N,N-diisopropylethylamine (0.22 mL, 1.3 mmol) was added and the reaction mixture was allowed to warm to room temperature and stirred for 2 hours. (R)-1-(4-fluorophenyl)-1,2,3,4-tetrahydroisoquinoline (0.26 g, 1.1 mmol) and N,N-diisopropylethylamine (0.22 mL, 1.3 mmol) were added and the reaction mixture was stirred for 3 days. The mixture was concentrated to dryness under reduced pressure. The residue was purified by flash column chromatography (silica, 0 to 100% ethyl acetate in heptane) to give (R)-1-(tert-butoxycarbonyl)pyrrolidin-3-yl (R)-1-(4-fluorophenyl)-3,4-dihydroisoquinoline-2(1H)-carboxylate. LCMS: RT=2.29 min., (M+Na)⁺=463 (method B).

Synthesis of (R)-pyrrolidin-3-yl (R)-1-(4-fluorophenyl)-3,4-dihydroisoquinoline-2(1H)-carboxylate (COMPOUND 2266).

Starting from (R)-1-(tert-butoxycarbonyl)pyrrolidin-3-yl (R)-1-(4-fluorophenyl)-3,4-dihydroisoquinoline-2(1H-carboxylate (73 mg, 0.17 mmol), (R)-pyrrolidin-3-yl (R)-1-(4-fluorophenyl)-3,4-dihydroisoquinoline-2(1H-carboxylate (COMPOUND 2266, 20 mg) was prepared according to the procedure described for (R)-pyrrolidin-3-yl (S)-1-(3-fluorobicyclo[1.1.1]pentan-1-yl)-3,4-dihydroisoquinoline-2(1H-carboxylate (COMPOUND 2290) and desalted (SCX-2 (5 g) ion exchange chromatography). LCMS: RT=2.63 min., (M+H)⁺=341 (method AK). Chiral LC: RT=7.70 min. (method AB).

### ((2R,3R,4R)-3,4-dihydroxy-1-methylpiperidin-2-yl)methyl (S)-1-(4-fluorophenyl)-3,4-dihydroisoquinoline-2(1H)-carboxylate (Compound 2289)

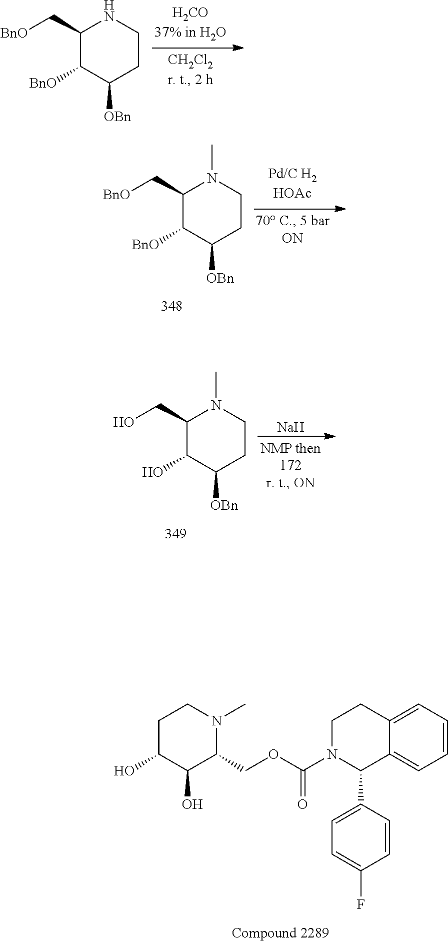

Synthesis of (2R,3R,4R)-3,4-bis(benzyloxy)-2-((benzyloxy)methyl)-1-methylpiperidine.

Formaldehyde (37 wt % solution in water, stabilized with 10-15% methanol, 0.392 mL, 5.22 mmol) followed by sodium triacetoxyborohydride (1.107 g, 5.22 mmol) was added to a solution of (2R,3R,4R)-3,4-bis(benzyloxy)-2-((benzyloxy)methyl)piperidine (1.09 g, 2.61 mmol) in dichloromethane (12 mL). The mixture was stirred at room temperature for 2 hours. The reaction mixture was partitioned between dichloromethane (10 mL) and a mixture of saturated aqueous K₂CO₃ solution and water (1:1, 10 mL). The layers were separated using a phase separator and the organic filtrate was evaporated under reduced pressure. The residue was purified by flash column chromatography (silica, 0 to 10% methanol in dichloromethane) to give (2R,3R,4R)-3,4-bis(benzyloxy)-2-((benzyloxy)methyl)-1-methylpiperidine. LCMS: RT=2.36 min., (M+H)⁺=432 (method B).

Synthesis of (2R,3R,4R)-2-(hydroxymethyl)-1-methylpiperidine-3,4-diol.

Palladium on carbon (10 wt %, containing 50% water, 0.46 g, 0.216 mmol) was added to a degassed solution of (2R,3R,4R)-3,4-bis(benzyloxy)-2-((benzyloxy)methyl)-1-methylpiperidine (0.93 g, 2.155 mmol) in acetic acid (20 mL). The mixture was hydrogenated at 70° C. and 5 bar overnight, then allowed to cool to room temperature, flushed with nitrogen and filtered over a layer of celite in a glass filter. The residue was washed with a mixture of water and ethanol (3:1). The combined filtrates were concentrated under reduced pressure and then evaporated from water (50 mL) and ethanol (3×50 mL). The residue was dissolved in methanol (10 mL) and sodium methoxide (35 wt % solution in methanol, 0.4 mL, 2.16 mmol) was added. The mixture was stirred for 1 hour, then the pH of the mixture was adjusted to 7 by addition of aqueous HCl (1 M), and the resultant mixture was concentrated under reduced pressure. The residue was dissolved in water (10 mL) and brought onto a column containing Dowex® 50WX8 50-100 mesh (about 100 mL). The column was eluted with water until neutral eluate and then eluted with aqueous ammonia (1 M). The product containing fractions were combined and evaporated under reduced pressure giving (2R,3R,4R)-2-(hydroxymethyl)-1-methylpiperidine-3,4-diol.

Synthesis of ((2R,3R,4R)-3,4-dihydroxy-1-methylpiperidin-2-yl)methyl (S)-1-(4-fluorophenyl)-3,4-dihydroisoquinoline-2(1H)-carboxylate (COMPOUND 2289).

Sodium hydride (60 wt % dispersion in mineral oil, 2.481 mg, 0.062 mmol) was added to (2R,3R,4R)-2-(hydroxymethyl)-1-methylpiperidine-3,4-diol (10 mg, 0.062 mmol) in N-methyl-2-pyrrolidinone (dry, 0.2 mL). After 10 minutes, (S)-1-(4-fluorophenyl)-3,4-dihydroisoquinoline-2(1H)-carbonyl chloride (see COMPOUND 2324, 18.8 mg, 0.065 mmol) was added. The mixture was stirred for 4 hours, then diluted with methanol (1 mL) and purified by basic preparative MPLC (Linear Gradient: t=0 min 5% A, t=1 min 5% A; t=1 min 20% A; t=15 min 60% A; t=1 min 100% A; t=5 min 100% A; detection: 210/264 nm). The product containing fractions were concentrated under reduced pressure and the residue was lyophilized from a mixture of acetonitrile and water (1:1, 4 mL) to give ((2R,3R,4R)-3,4-dihydroxy-1-methylpiperidin-2-yl)methyl (S)-1-(4-fluorophenyl)-3,4-dihydroisoquinoline-2(1H)-carboxylate (COMPOUND 2289). LCMS: RT=1.45 min., (M+H)⁺=415 (method Q).

### ((2S,3S,4S)-3,4-dihydroxy-1-methylpiperidin-2-yl)methyl (S)-1-(4-fluorophenyl)-3,4-dihydroisoquinoline-2(1H-carboxylate (Compound 2304) and

### (2S,3S,4S)-4-hydroxy-2-(hydroxymethyl)-1-methylpiperidin-3-yl (S)-1-(4-fluorophenyl)-3,4-dihydroisoquinoline-2(1H)-carboxylate (Compound 2305)

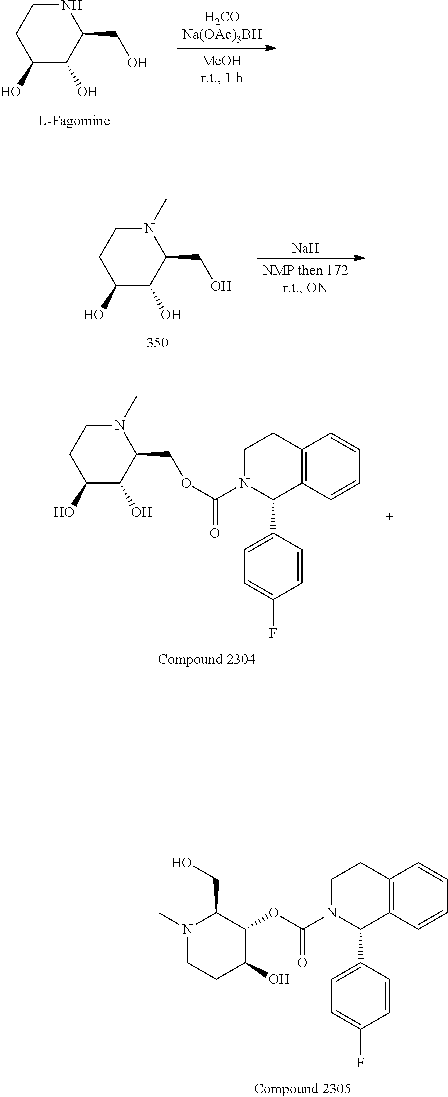

Synthesis of (2S,3S,4S)-2-(hydroxymethyl)-1-methylpiperidine-3,4-diol.

Formaldehyde (37 wt % solution in water, stabilized with 10-15% methanol, 0.225 mL, 2.99 mmol) followed by sodium triacetoxyborohydride (634 mg, 2.99 mmol) was added to a solution of (2S,3S,4S)-2-(hydroxymethyl)piperidine-3,4-diol (88 mg, 0.598 mmol) in methanol (5 mL). The reaction mixture was stirred at room temperature. After 1 hour, the reaction mixture was diluted with water (10 mL) and the methanol was removed under reduced pressure. The residue was brought onto a column containing Dowex® 50WX8 50-100 mesh (H⁺-form, about 50 mL). The column was eluted with water until neutral and then with aqueous ammonia (1 M). The product containing fractions were concentrated under reduced pressure giving (2S,3S,4S)-2-(hydroxymethyl)-1-methylpiperidine-3,4-diol.

Synthesis of ((2S,3S,4S)-3,4-dihydroxy-1-methylpiperidin-2-yl)methyl (S)-1-(4-fluorophenyl)-3,4-dihydroisoquinoline-2(1H)-carboxylate (COMPOUND 2304) and (2S,3S,4S)-4-hydroxy-2-(hydroxymethyl)-1-methylpiperidin-3-yl (S)-1-(4-fluorophenyl)-3,4-dihydroisoquinoline-2(1H)-carboxylate (COMPOUND 2305).

Starting from (2S,3S,4S)-2-(hydroxymethyl)-1-methylpiperidine-3,4-diol (88 mg, 0.546 mmol) and (S)-1-(4-fluorophenyl)-3,4-dihydroisoquinoline-2(1H)-carbonyl chloride (see COMPOUND 2324, 158 mg, 0.546 mmol), (2S,3S,4S)-4-hydroxy-2-(hydroxymethyl)-1-methylpiperidin-3-yl (S)-1-(4-fluorophenyl)-3,4-dihydroisoquinoline-2(1H)-carboxylate (COMPOUND 2305) as the first eluting SFC isomer and ((2S,3S,4S)-3,4-dihydroxy-1-methylpiperidin-2-yl)methyl (S)-1-(4-fluorophenyl)-3,4-dihydroisoquinoline-2(1H-carboxylate (COMPOUND 2304) as the second eluting SFC isomer were prepared as described for ((2R,3R,4R)-3,4-dihydroxy-1-methylpiperidin-2-yl)methyl (S)-1-(4-fluorophenyl)-3,4-dihydroisoquinoline-2(1H-carboxylate (COMPOUND 2289). After workup, the molecules were purified by basic reverse phase MPLC (Linear Gradient: t=0 min 5% A, t=1 min 5% A; t=1 min 20% A; t=15 min 60% A; t=1 min 100% A t=5 min 100% A; detection: 210/264 nm) followed by chiral SFC (Method S). COMPOUND 2304: LCMS: RT=1.45 min., (M+H)⁺=415 (method Q). COMPOUND 2305: LCMS: RT=1.49 min., (M+H)⁺=415 (method Q).

### (3R,4R)-4-fluoropyrrolidin-3-yl (S)-1-(4-fluorophenyl)-3,4-dihydroisoquinoline-2(1H-carboxylate (Compound 2349)

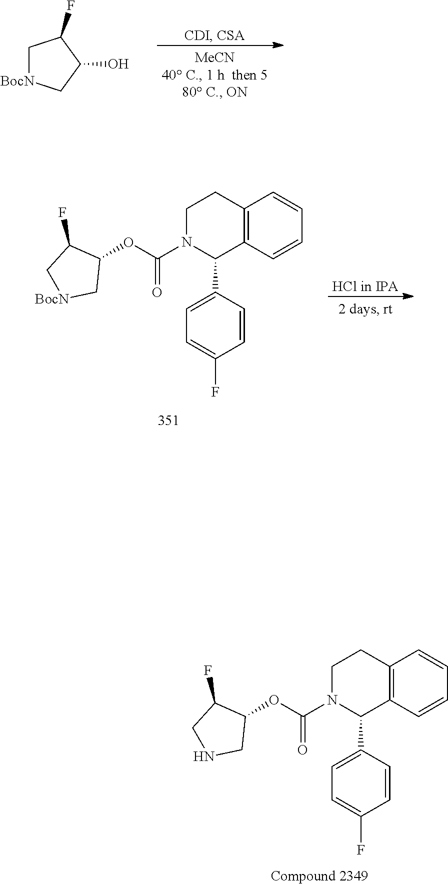

Synthesis of (3R,4R)-1-(tert-butoxycarbonyl)-4-fluoropyrrolidin-3-yl (S)-1-(4-fluorophenyl)-3,4-dihydroisoquinoline-2(1H)-carboxylate.

Under argon atmosphere, 1,1′-carbonyldiimidazole (43.5 mg, 0.268 mmol) was added to a suspension of tert-butyl (3R,4R)-3-fluoro-4-hydroxypyrrolidine-1-carboxylate (50 mg, 0.244 mmol) in acetonitrile (2 mL) and the reaction mixture was stirred at 40° C. for 1 hour. At room temperature, (S)-1-(4-fluorophenyl)-1,2,3,4-tetrahydroisoquinoline (60.9 mg, 0.268 mmol) followed by DL-10-camphorsulfonic acid (102 mg, 0.439 mmol) were added and the resulting mixture was stirred at 80° C. overnight. The reaction mixture was concentrated under reduced pressure. The residue was diluted with dichloromethane (10 mL) and saturated aqueous NaHCO₃ (15 mL). The aqueous layer was extracted with dichloromethane (3×10 mL). The combined organics were dried over Na₂SO₄ and evaporated under reduced pressure. The residue was purified by flash column chromatography (silica, 0 to 30% ethyl acetate in heptane) to give (3R,4R)-1-(tert-butoxycarbonyl)-4-fluoropyrrolidin-3-yl (S)-1-(4-fluorophenyl)-3,4-dihydroisoquinoline-2(1H-carboxylate. LCMS: RT=2.33 min., (M−tBu+H)⁺=403 (method B).

Synthesis of (3R,4R)-4-fluoropyrrolidin-3-yl (S)-1-(4-fluorophenyl)-3,4-dihydroisoquinoline-2(1H)-carboxylate (COMPOUND 2349).

Starting from (3R,4R)-1-(tert-butoxycarbonyl)-4-fluoropyrrolidin-3-yl (S)-1-(4-fluorophenyl)-3,4-dihydroisoquinoline-2(1H-carboxylate (70 mg, 0.153 mmol), (3R,4R)-4-fluoropyrrolidin-3-yl (S)-1-(4-fluorophenyl)-3,4-dihydroisoquinoline-2(1H)-carboxylate (COMPOUND 2349) was prepared as described for (S)—N-((trans)-3-amino-1-methylcyclobutyl)-1-(4-fluorophenyl)-3,4-dihydroisoquinoline-2(1H)-carboxamide (COMPOUND 2271) and eluted through an SCX-2 column (1 g). LCMS: RT=1.08 min., (M+H)⁺=359 (method P).

### (3S,4S)-4-fluoropyrrolidin-3-yl (S)-1-(4-fluorophenyl)-3,4-dihydroisoquinoline-2(1H)-carboxylate (Compound 2361)

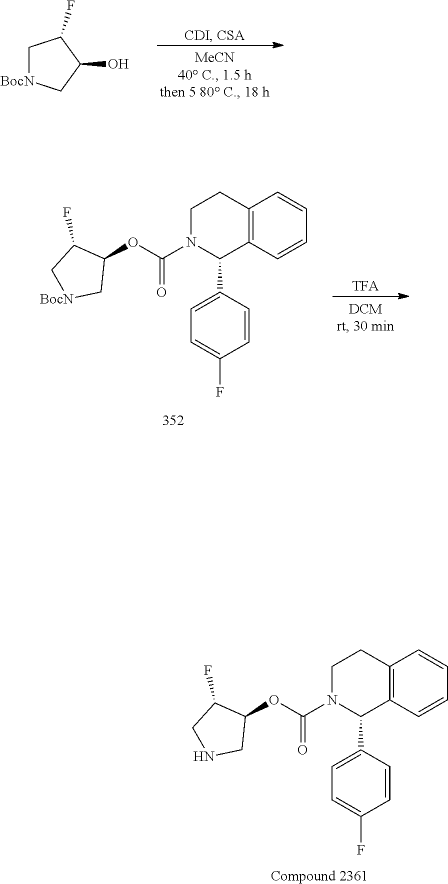

Synthesis of (3S,4S)-1-(tert-butoxycarbonyl)-4-fluoropyrrolidin-3-yl (S)-1-(4-fluorophenyl)-3,4-dihydroisoquinoline-2(1H)-carboxylate.

Starting from tert-butyl (3S,4S)-3-fluoro-4-hydroxypyrrolidine-1-carboxylate (50.0 mg, 0.244 mmol) and (S)-1-(4-fluorophenyl)-1,2,3,4-tetrahydroisoquinoline (60.9 mg, 0.268 mmol), (3S,4S)-1-(tert-butoxycarbonyl)-4-fluoropyrrolidin-3-yl (S)-1-(4-fluorophenyl)-3,4-dihydroisoquinoline-2(1H-carboxylate was prepared as described for (3R,4R)-1-(tert-butoxycarbonyl)-4-fluoropyrrolidin-3-yl (S)-1-(4-fluorophenyl)-3,4-dihydroisoquinoline-2(1H-carboxylate (see COMPOUND 2349). LCMS: RT=2.31 min., (M−tBu+H)⁺=403 (method B).

Synthesis of (3S,4S)-4-fluoropyrrolidin-3-yl (S)-1-(4-fluorophenyl)-3,4-dihydroisoquinoline-2(1H)-carboxylate (COMPOUND 2361).

Starting from (3S,4S)-1-(tert-butoxycarbonyl)-4-fluoropyrrolidin-3-yl (S)-1-(4-fluorophenyl)-3,4-dihydroisoquinoline-2(1H-carboxylate (89.2 mg (92 wt %), 0.179 mmol), (3S,4S)-4-fluoropyrrolidin-3-yl (S)-1-(4-fluorophenyl)-3,4-dihydroisoquinoline-2(1H-carboxylate (COMPOUND 2361) was prepared as described for (R)-pyrrolidin-3-yl (R)-1-(4-fluorophenyl)-3,4-dihydroisoquinoline-2(1H-carboxylate (COMPOUND 2266) using a mixture of saturated aqueous NaHCO₃ and saturated aqueous K₂CO₃ (2:1, 15 mL) for the basic workup. LCMS: RT=1.08 min., (M+H)⁺=359 (method P). SFC: partial epimerisation observed (non-baseline separation), RT=2.70 min., (M+H)⁺=359 (method AR).

Compounds 2362 and 2363 were prepared by a similar synthesis as Compound 2361: COMPOUND 2362 (M+H)⁺=359. RT=1.10 min., (method P)

COMPOUND 2363 (M+H)⁺=359. RT=1.10 min., (method P)

### 2-((S)-quinuclidin-3-yl)ethyl (S)-1-(4-fluorophenyl)-3,4-dihydroisoquinoline-2(1H)-carboxylate formate (Compound 2206)

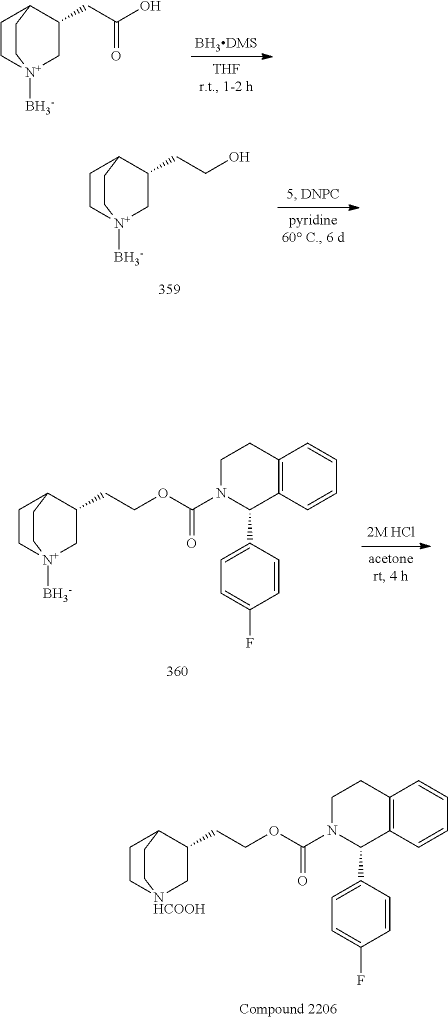

Synthesis of ((S)-2-(quinuclidin-3-yl)ethan-1-ol)trihydroborate.

Under nitrogen atmosphere, borane dimethyl sulfide complex (2 M in tetrahydrofuran, 0.273 mL, 0.546 mmol) was added to a solution of (S)-(3-(carboxymethyl)quinuclidin-1-ium-1-yl)trihydroborate (50 mg, 0.273 mmol) in tetrahydrofuran (dry, 3 mL). After 2 hours, the reaction mixture was quenched by the addition of water and extracted with chloroform (twice). The combined organic layers were concentrated under reduced pressure to give ((S)-2-(quinuclidin-3-yl)ethan-1-ol)trihydroborate

Synthesis of (2-((S)-quinuclidin-3-yl)ethyl (S)-1-(4-fluorophenyl)-3,4-dihydroisoquinoline-2(1H)-carboxylate)trihydroborate.

Starting from ((S)-2-(quinuclidin-3-yl)ethan-1-ol)trihydroborate (47 mg, 0.278 mmol) and (S)-1-(4-fluorophenyl)-1,2,3,4-tetrahydroisoquinoline (63.2 mg, 0.278 mmol), (2-((S)-quinuclidin-3-yl)ethyl (S)-1-(4-fluorophenyl)-3,4-dihydroisoquinoline-2(1H-carboxylate)trihydroborate was prepared, at 60° C., as described for tert-butyl (S)-7-(benzyloxy)-1-(4-fluorophenyl)-3,4-dihydroisoquinoline-2(1H)-carboxylate (see COMPOUND 2115). The molecule was purified by acidic preparative MPLC (Linear Gradient: t=0 min 5% A, t=1 min 5% A; t=2 min 20% A, t=17 min 60% A, t=18 min 100% A, t=24 min 100% A; detection: 220/270/288 nm). LCMS: RT=2.21 min., (M+Na)⁺=445 (method A).

Synthesis of 2-((S)-quinuclidin-3-yl)ethyl (S)-1-(4-fluorophenyl)-3,4-dihydroisoquinoline-2(1H)-carboxylate formate (COMPOUND 2206).

Aqueous HCl (2 M, 0.092 mL, 0.185 mmol) was added to a solution of (2-((S)-quinuclidin-3-yl)ethyl (S)-1-(4-fluorophenyl)-3,4-dihydroisoquinoline-2(1H)-carboxylate)trihydroborate (26 mg, 0.062 mmol) in acetone (1 mL) and the reaction mixture was stirred for 3 hours. Additional aqueous HCl (2 M, 0.154 mL, 0.308 mmol) was added and the reaction mixture was stirred for an additional hour. The reaction mixture was concentrated to dryness under reduced pressure. The residue was purified twice by acidic preparative MPLC (Linear Gradient: t=0 min 5% A, t=1 min 5% A; t=2 min 10% A, t=17 min 50% A, t=18 min 100% A, t=23 min 100% A; detection: 220/254/270 nm) to give 2-((S)-quinuclidin-3-yl)ethyl (S)-1-(4-fluorophenyl)-3,4-dihydroisoquinoline-2(1H)-carboxylate formate (COMPOUND 2206) after lyophilisation from a mixture of acetonitrile and water (1:1, 4 mL). LCMS: 99%, RT=2.98 min., (M+H)⁺=409 (method C).

Cellular Assays: To measure the efficacy of compounds, a progranulin induction cellular assay in mouse primary microglia (pMG), primary cortical neurons, and BV-2 cell lines is used. BV-2 cells are split the day before plating into a 96 well plate format at approximately 80%. Cells should be plated the day before and allowed for 1 hour attachment period and for 16 hour incubation. Levels of progranulin secreted into the cell culture medium or retained in the cell lysate can be quantified using an ELISA-based readout and measurement of secreted mouse PGRN in the medium was assessed by the methodology published by Ghidoni et al. 2012. Standard ELISA kits to measure PGRN are available from vendors such as Adipogen, R&D, and Biovendor.

In vivo Assays: A mouse ELISA protocol to detect progranulin in brain, plasma, or cerebrospinal fluid (CSF) can be used, with GRN+/− mice or GRN+/+ mice (available from TACONIC). The mouse is administered a compound as disclosed herein and the amount of progranulin in the brain is assessed after a specific amount of time. Mice treated with a test compound or compounds are compared to control mice which are not treated with the compound. Treatment can be done with a single or multiple dosing of compounds. Control samples are assigned a relative value of 100%.

Other in vivo assays can be performed using a GRN+/− and GRN+/+ rats, non-human primates (e.g., monkey, dog) using a similar protocol.

Treatment with the test compound increases the progranulin secretion relative to the control is at least about 110%, at least about 130%, at least about 150%, at least about 180%, at least about 200%, at least about 250%, or at least about 300%.

Tables C and D below present the results of a PGRN assay as described above. Table C

TABLE C

| Compound No. | EC₅₀ μM |
|---|---|
| Compound 2001 | 0.17 |
| Compound 2002 | 0.748 |
| Compound 2003 | 1.13 |
| Compound 2004 | 1.05 |
| Compound 2005 | 0.587 |
| Compound 2006 | >10.0 |
| Compound 2007 | 1.19 |
| Compound 2008 | 0.559 |
| Compound 2009 | 0.811 |
| Compound 2010 | >10.0 |
| Compound 2011 | >10.0 |
| Compound 2012 | >10.0 |
| Compound 2013 | >10.0 |
| Compound 2014 | 2.32 |
| Compound 2015 | 1.5 |
| Compound 2016 | 0.581 |
| Compound 2017 | 0.722 |
| Compound 2018 | 1.89 |
| Compound 2019 | 0.965 |
| Compound 2020 | 0.23 |
| Compound 2021 | 0.735 |
| Compound 2022 | 0.96 |
| Compound 2023 | 1.18 |
| Compound 2024 | >10.0 |
| Compound 2025 | >10.0 |
| Compound 2026 | >10.0 |
| Compound 2027 | >10.0 |
| Compound 2028 | >10.0 |
| Compound 2029 | >10.0 |
| Compound 2030 | >10.0 |
| Compound 2031 | 5.75 |
| Compound 2032 | >10.0 |
| Compound 2033 | >10.0 |
| Compound 2034 | 6.07 |
| Compound 2035 | >10.0 |
| Compound 2036 | >10.0 |
| Compound 2037 | >8.09 |
| Compound 2038 | >10.0 |
| Compound 2039 | >10.0 |
| Compound 2040 | >10.0 |
| Compound 2041 | >10.0 |
| Compound 2042 | 3.20 |
| Compound 2043 | >10.0 |
| Compound 2044 | 4.71 |
| Compound 2045 | >10.0 |
| Compound 2046 | 1.72 |
| Compound 2047 | >2.69 |
| Compound 2048 | 1.9 |
| Compound 2049 | 1.55 |
| Compound 2050 | 3.9 |
| Compound 2051 | >10.0 |
| Compound 2052 | 6.82 |
| Compound 2053 | 5.74 |
| Compound 2054 | 4.74 |
| Compound 2055 | 3.3 |
| Compound 2056 | >10.0 |
| Compound 2057 | >10.0 |
| Compound 2058 | 3 |
| Compound 2059 | >10.0 |
| Compound 2060 | >10.0 |
| Compound 2061 | 0.321 |
| Compound 2062 | 1.12 |
| Compound 2063 | >10.0 |
| Compound 2064 | >10.0 |
| Compound 2065 | >10.0 |
| Compound 2066 | >10.0 |
| Compound 2067 | 0.24 |
| Compound 2068 | 3.37 |
| Compound 2069 | 4.13 |
| Compound 2070 | 3.62 |
| Compound 2071 | 3.72 |
| Compound 2072 | 8.08 |
| Compound 2073 | >10.0 |
| Compound 2074 | 3.4 |
| Compound 2075 | 0.895 |
| Compound 2076 | >10.0 |
| Compound 2077 | >10.0 |
| Compound 2078 | 5.35 |
| Compound 2079 | 4.83 |
| Compound 2080 | >10.0 |
| Compound 2081 | >10.0 |
| Compound 2082 | 7.46 |
| Compound 2083 | 6.07 |
| Compound 2084 | >10.0 |
| Compound 2085 | 4.99 |
| Compound 2086 | >10.0 |
| Compound 2087 | >10.0 |
| Compound 2088 | >10.0 |
| Compound 2089 | >10.0 |
| Compound 2090 | >10.0 |
| Compound 2091 | >10.0 |
| Compound 2092 | >10.0 |
| Compound 2093 | 0.379 |
| Compound 2094 | 7.22 |
| Compound 2095 | 3.17 |
| Compound 2096 | 0.387 |
| Compound 2097 | 2.82 |
| Compound 2098 | 0.339 |
| Compound 2099 | 1.88 |
| Compound 2100 | >10.0 |
| Compound 2101 | >10.0 |
| Compound 2102 | 0.278 |
| Compound 2103 | 4.28 |
| Compound 2104 | 1.61 |
| Compound 2105 | 1.07 |
| Compound 2106 | >10.0 |
| Compound 2107 | >10.0 |
| Compound 2108 | >3.16 |
| Compound 2109 | >10.0 |
| Compound 2110 | 7.82 |
| Compound 2111 | 0.635 |
| Compound 2112 | 8.27 |
| Compound 2113 | 0.596 |
| Compound 2114 | 0.401 |
| Compound 2115 | 8.37 |
| Compound 2116 | >10.0 |
| Compound 2117 | >10.0 |
| Compound 2118 | >10.0 |
| Compound 2119 | >10.0 |
| Compound 2120 | 2.68 |
| Compound 2121 | >10.0 |
| Compound 2122 | 3.28 |
| Compound 2123 | >10.0 |
| Compound 2124 | 5.34 |
| Compound 2125 | >3.16 |
| Compound 2126 | 1.25 |
| Compound 2127 | 1.86 |
| Compound 2128 | 7.54 |
| Compound 2129 | 0.358 |
| Compound 2130 | >3.16 |
| Compound 2131 | 4.19 |
| Compound 2132 | 3.07 |
| Compound 2133 | 9.76 |
| Compound 2134 | >10.0 |
| Compound 2135 | 2.66 |
| Compound 2136 | >3.16 |
| Compound 2137 | >3.16 |
| Compound 2138 | 1.21 |
| Compound 2139 | >10.0 |
| Compound 2140 | 9.91 |
| Compound 2141 | >10.0 |
| Compound 2142 | 8.49 |
| Compound 2143 | 7.75 |
| Compound 2144 | 1.34 |
| Compound 2145 | 2.93 |
| Compound 2146 | 1.61 |
| Compound 2147 | 4.74 |
| Compound 2148 | 8.83 |
| Compound 2149 | >10.0 |
| Compound 2150 | 9.46 |
| Compound 2151 | 1.16 |
| Compound 2152 | 6.4 |
| Compound 2153 | >10.0 |
| Compound 2154 | 0.353 |
| Compound 2155 | >10.0 |
| Compound 2156 | 0.713 |
| Compound 2161 | >10.0 |
| Compound 2162 | >10.0 |
| Compound 2163 | >10.0 |
| Compound 2165 | >10.0 |
| Compound 2168 | 2.54 |
| Compound 2170 | 1.59 |
| Compound 2171 | 0.13 |
| Compound 2172 | 0.43 |
| Compound 2173 | 3.22 |
| Compound 2174 | 2.15 |
| Compound 2175 | 4.13 |
| Compound 2176 | 0.29 |
| Compound 2177 | >3.16 |
| Compound 2178 | 0.59 |
| Compound 2179 | 0.12 |
| Compound 2180 | 0.083 |
| Compound 2181 | 0.22 |
| Compound 2182 | >10.0 |
| Compound 2183 | 9.60 |
| Compound 2184 | >10.0 |
| Compound 2185 | >10.0 |
| Compound 2186 | >10.0 |
| Compound 2187 | 0.40 |
| Compound 2188 | 3.21 |
| Compound 2189 | 3.32 |
| Compound 2192 | >10.0 |
| Compound 2193 | 2.51 |
| Compound 2194 | 3.2 |
| Compound 2195 | 1.7 |
| Compound 2196 | >10.0 |
| Compound 2197 | 1.06 |
| Compound 2198 | >10.0 |

TABLE D

| Compound No. | EC₅₀ μM |
|---|---|
| Compound 2203 | 0.27 |
| Compound 2204 | 0.65 |
| Compound 2206 | 1.99 |
| Compound 2208 | 7.22 |
| Compound 2209 | 0.73 |
| Compound 2210 | 0.86 |
| Compound 2211 | 3.03 |
| Compound 2212 | 3.05 |
| Compound 2213 | 3.53 |
| Compound 2214 | 3.61 |
| Compound 2215 | 2.7 |
| Compound 2216 | 2.67 |
| Compound 2217 | 3.94 |
| Compound 2218 | 3.95 |
| Compound 2219 | 6.21 |
| Compound 2220 | 5.5 |
| Compound 2221 | 7.83 |
| Compound 2222 | 0.94 |
| Compound 2223 | 1.58 |
| Compound 2224 | 0.48 |
| Compound 2225 | 1.01 |
| Compound 2226 | 2.68 |
| Compound 2227 | 4.15 |
| Compound 2230 | 1.07 |
| Compound 2231 | 1.04 |
| Compound 2237 | 0.43 |
| Compound 2238 | 0.72 |
| Compound 2239 | 1.14 |
| Compound 2240 | 2.39 |
| Compound 2241 | 8.79 |
| Compound 2242 | 3.95 |
| Compound 2243 | 0.36 |
| Compound 2244 | 3.26 |
| Compound 2245 | 0.46 |
| Compound 2246 | 0.77 |
| Compound 2247 | >10.0 |
| Compound 2248 | 1.24 |
| Compound 2249 | >10.0 |
| Compound 2250 | 0.1 |
| Compound 2251 | 0.11 |
| Compound 2252 | 0.12 |
| Compound 2253 | 0.11 |
| Compound 2254 | 2.91 |
| Compound 2255 | 0.24 |
| Compound 2256 | 0.17 |
| Compound 2257 | 0.55 |
| Compound 2258 | 0.27 |
| Compound 2259 | >10.0 |
| Compound 2260 | 0.73 |
| Compound 2261 | >10.0 |
| Compound 2262 | >10.0 |
| Compound 2263 | 1.28 |
| Compound 2264 | 1.06 |
| Compound 2265 | 0.286 |
| Compound 2266 | 0.255 |
| Compound 2267 | 0.13 |
| Compound 2268 | 0.18 |
| Compound 2269 | 0.656 |
| Compound 2270 | 0.515 |
| Compound 2272 | 2.03 |
| Compound 2273 | 0.319 |
| Compound 2274 | 1.4 |
| Compound 2275 | 0.447 |
| Compound 2276 | 1.05 |
| Compound 2277 | 1.06 |
| Compound 2278 | 0.439 |
| Compound 2279 | 0.428 |
| Compound 2280 | 1.18 |
| Compound 2281 | 0.273 |
| Compound 2282 | >10.0 |
| Compound 2283 | >10.0 |
| Compound 2284 | 0.806 |
| Compound 2285 | 3.44 |
| Compound 2286 | 2.84 |
| Compound 2287 | 0.351 |
| Compound 2288 | 0.248 |
| Compound 2289 | >10.0 |
| Compound 2291 | 0.591 |
| Compound 2292 | 0.431 |
| Compound 2293 | >10.0 |
| Compound 2294 | >10.0 |
| Compound 2296 | 4.02 |
| Compound 2297 | >10.0 |
| Compound 2298 | >10.0 |
| Compound 2299 | >10.0 |
| Compound 2302 | 0.16 |
| Compound 2303 | 0.329 |
| Compound 2304 | 2.33 |
| Compound 2305 | 5.87 |
| Compound 2306 | 0.129 |
| Compound 2307 | 0.483 |
| Compound 2308 | 2.94 |
| Compound 2309 | 0.6 |
| Compound 2310 | 0.501 |
| Compound 2311 | 1.32 |
| Compound 2312 | 0.987 |
| Compound 2313 | 2.94 |
| Compound 2314 | 1.66 |
| Compound 2315 | 0.742 |
| Compound 2316 | 0.453 |
| Compound 2317 | 9.74 |
| Compound 2318 | 3.35 |
| Compound 2323 | 8.87 |
| Compound 2324 | 0.742 |
| Compound 2325 | 4.7 |
| Compound 2326 | 0.189 |
| Compound 2349 | 0.346 |
| Compound 2351 | 0.13 |
| Compound 2352 | 0.125 |
| Compound 2353 | 0.0339 |
| Compound 2360 | 0.148 |
| Compound 2361 | 0.365 |
| Compound 2362 | >3.16 |
| Compound 2363 | 0.104 |

In view of the many possible embodiments to which the principles of the disclosure may be applied, it should be recognized that the illustrated embodiments are only examples and should not be taken as limiting the scope of the invention.

What is claimed:

1. A compound, or pharmaceutically acceptable salt thereof, having a structure of Formula (I): 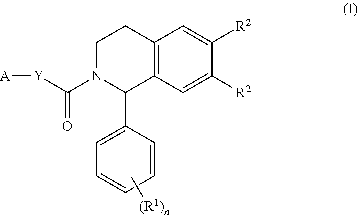
wherein
A is a 4-10 membered heterocycle comprising 1 to 3 ring heteroatoms selected from N, O, and S, optionally substituted with 1 to 3 R³;
Y is C₀₋₆alkylene, C₀₋₆alkylene-O—C₀₋₆alkylene, C₀₋₆alkylene-NR^{N}, C₀₋₆alkylene-SO₂, CO, CO₂, or CONH, wherein C₀₋₆alkylene is optionally substituted with 1 or 2 R⁴;
each R¹ is independently halo, —O—CH₂—C₆aryl-(OCH₂CH₂)ₚ—OR⁵, or —O—CH₂—Het-(OCH₃), wherein Het is a 6-membered heteroaryl comprising 2 ring N atoms;
one R² is H and the other is H, CN, COOC₁₋₆alkyl, CONHC₁₋₆alkyl, SO₂CH₃, or O-propargyl;
R³ is C₁₋₆alkyl, halo, C₀₋₆alkylene-OH, C₀₋₆alkylene-O-propargyl, propargyl, or C₀₋₆alkylene-NR^{N}R^{N};
each R⁴ is independently F, OH, or OC₁₋₆alkyl, or two R⁴ together with the carbon atom to which they are attached form cyclopropyl;
R⁵ is C₁₋₆alkyl or propargyl;
each R^{N} is independently H or C₁₋₆alkyl;
n is 1-3; and
p is 0-2;
with the proviso that
(a) if A comprises, 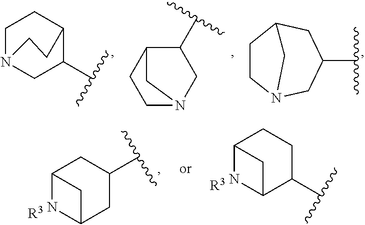
and Y is CH₂, O, or NR^{N}, then one R² is H and the other R² is not H; or
(b) if Y is CH₂, O, or NR^{N} and each R² is H, then A does not comprise

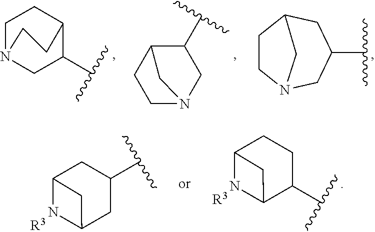

2. The compound or salt of claim 1, having a structure of Formula (Ia) or (Ib):

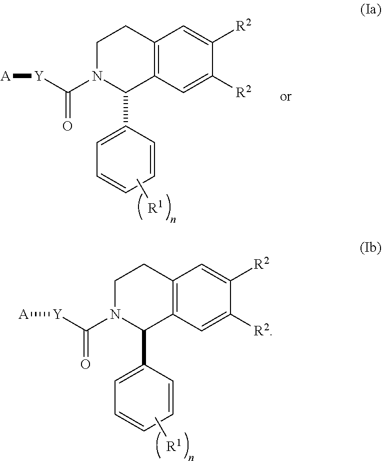

3. The compound or salt of claim 1, having a structure of Formula (Ic) or (Id):

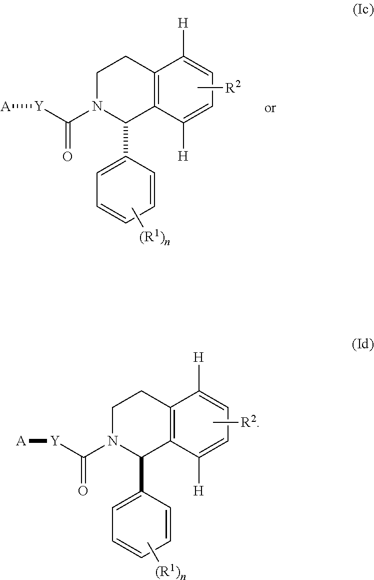

4. The compound or salt of any one of claims 1 to 3, wherein A comprises a 4-, 6-, 8-, or 10-membered heterocycle comprising 1 or 2 ring heteroatoms selected from N and O.

5. The compound or salt of claim 4, wherein A comprises an 8-membered heterocycle comprising 1 or 2 ring heteroatoms selected from N and O.

6. The compound or salt of any one of claims 1 to 5, wherein A comprises

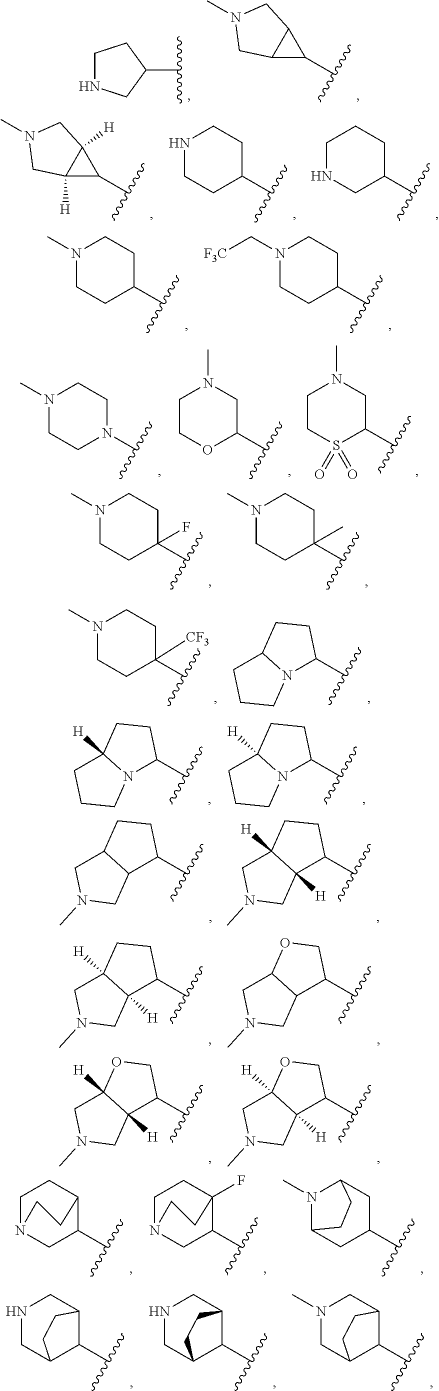

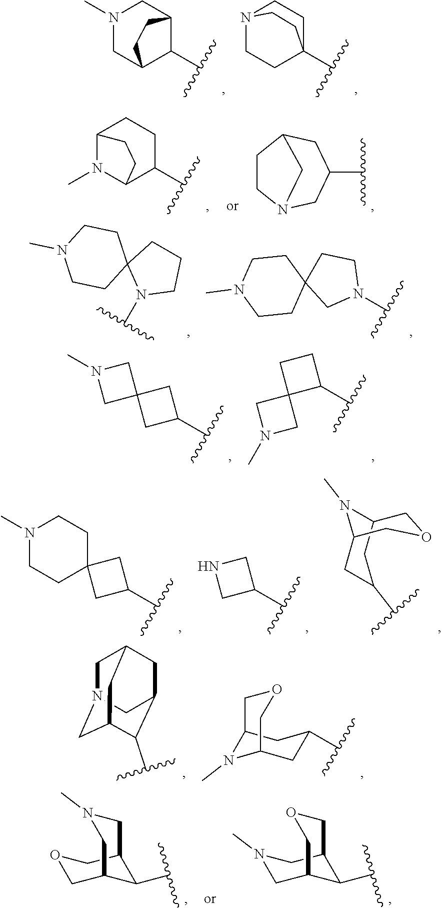

7. The compound or salt of any one of claims 1 to 6, wherein A comprises

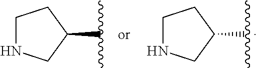 [SMILES: *[C@@H]1CCNC1 ; *[C@H]1CCNC1]

8. The compound or salt of any one of claims 1 to 6, wherein A comprises

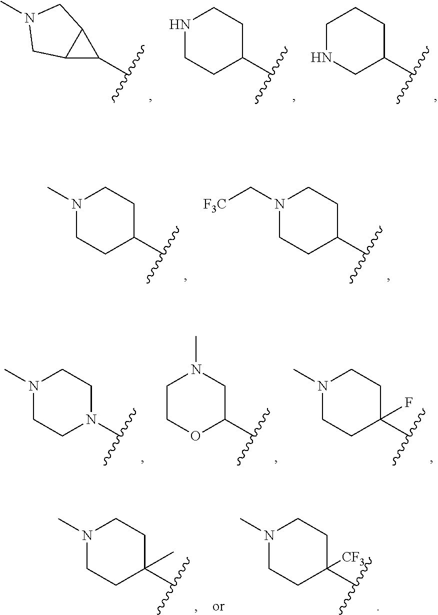

9. The compound or salt of any one of claims 1 to 6, wherein A comprises

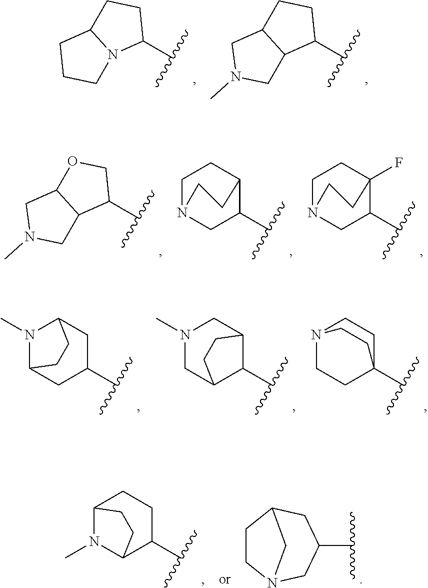

10. The compound or salt of claim 9, wherein A comprises

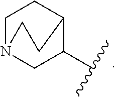 [SMILES: *C1CN2CCC1CC2]

11. The compound or salt of any one of claims 1 to 6, wherein A comprises

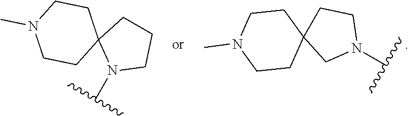 [SMILES: *N1CCCC12CCN(C)CC2 ; *N1CCC2(CCN(C)CC2)C1]

12. The compound or salt any one of claims 1 to 6, wherein A comprises

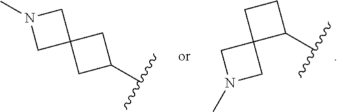

13. The compound or salt of any one of claims 1 to 6, wherein A comprises

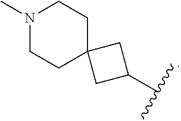 [SMILES: *C1CC2(CCN(C)CC2)C1]

14. The compound or salt of claims 1 to 13, wherein Y is NH or O.

15. The compound or salt of claim 14, wherein Y is NH.

16. The compound or salt of claim 14, wherein Y is O.

17. The compound or salt of any one of claims 1 to 13, wherein Y is C₀₋₆alkylene, C₁₋₆alkylene-O, C₁₋₆alkylene-NR^{N}, C₁₋₆alkylene-SO₂, CO₂, or CONH, and C₁₋₆alkylene is optionally substituted with 1 to 3 R⁴.

18. The compound or salt of claim 17, wherein Y is C₀ alkylene (i.e., a bond).

19. The compound or salt of claim 17, wherein Y is C₁₋₆alkylene, C₁₋₆alkylene-O, or C₁₋₆alkylene-NR³.

20. The compound or salt of claim 19, wherein Y is C₁₋₆alkylene-O.

21. The compound or salt of claim 19, wherein Y is C₁₋₆alkylene-NR^{N}.

22. The compound or salt of any one of claims 17 to 21, wherein C₁₋₆alkylene is substituted with 1 or 2 R⁴.

23. The compound or salt of claim 22, wherein R⁴ is OH or OCH₃.

24. The compound or salt of claim 23, wherein R⁴ is OH.

25. The compound or salt of claim 23, wherein R⁴ is OCH₃.

26. The compound or salt of any one of claims 1 to 25, wherein at least one R¹ is halo.

27. The compound or salt of claim 26, wherein at least one R¹ is F.

28. The compound or salt of claim 27, wherein each R¹ is F.

29. The compound or salt of any one of claims 1 to 25, wherein at least one R¹ is —O—CH₂—C₆aryl-(OCH₂CH₂)ₚ—OR⁵.

30. The compound or salt of claim 29, wherein p is 0.

31. The compound or salt of claim 29, wherein p is 1.

32. The compound or salt of claim 29, wherein p is 2.

33. The compound or salt of any one of claims 29 to 32, wherein R⁵ is methyl.

34. The compound or salt of any one of claims 29 to 32, wherein R⁵ is propargyl.

35. The compound or salt of any one of claims 1 to 25, wherein at least one R¹ is —O—CH₂—Het.

36. The compound or salt of claim 35, wherein Het comprises 2-pyrimidyl or 5-pyrimidyl optionally substituted with OMe.

37. The compound or salt of any one of claims 1 to 36, wherein n is 1.

38. The compound or salt of any one of claims 1 to 37, wherein n is 2.

39. The compound or salt of any one of claims 1 to 37, wherein n is 3.

40. The compound or salt of any one of claims 1 to 39, wherein R² is H.

41. The compound or salt of any one of claims 1 to 39, wherein R² is CN.

42. The compound or salt of any one of claims 1 to 39, wherein R² is COOCH₃ or CONHCH₃.

43. The compound or salt of claim 42, wherein R² is COOCH₃.

44. The compound or salt of claim 42, wherein R² is CONHCH₃.

45. The compound or salt of any one of claims 1 to 44, wherein R² is SO₂CH₃.

46. The compound or salt of any one of claims 1 to 44, wherein R² is O-propargyl.

47. A compound, or pharmaceutically acceptable salt thereof, having a structure as shown in Table A.

48. The compound or salt of claim 47, selected from the group consisting of Compound 2001, Compound 2171, Compound 2172, Compound 2176, Compound 2179, Compound 2180, Compound 2181, Compound 2243, Compound 2552, Compound 2250, Compound 2251, Compound 2253, and Compound 2254.

49. A compound, or pharmaceutically acceptable salt thereof, having a structure as shown in Table B.

50. The compound or salt of any one of claims 1 to 49 in the form of a salt.

51. A pharmaceutical composition comprising the compound of salt of any one of claims 1 to 50 and a pharmaceutically acceptable excipient.

52. Use of the compound or salt of any one of claims 1 to 50 as a medicament for the modulation of progranulin.

53. The use of claim 52, wherein progranulin secretion is increased.

54. A method of modulating progranulin in a subject in need thereof comprising administering to the subject the compound or salt of any one of claims 1 to 50 in an amount effective to increase progranulin secretion.

55. A method of treating a progranulin-associated disorder in a subject in need thereof comprising administering a therapeutically effective amount of the compound or salt of any one of claims 1 to 50 to the subject.

56. The method of claim 55, wherein the progranulin-associated disorder is Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), a lysosomal storage disease, a disease associated with inclusions and/or misfunction of C9orf72, TDP-43, FUS, UBQLN2, VCP, CHMP28, and/or MAPT, an acute neurological disorder, glioblastoma, or neuroblastoma.

57. The method of claim 56, wherein the lysosomal storage disease is Paget's disease, Gaucher's disease, Nieman's Pick disease, Tay-Sachs Disease, Fabry Disease, Pompes disease, or Naso-Hakula disease.

58. The method of claim 56, wherein the acute neurological disorder is stroke, cerebral hemorrhage, traumatic brain injury or head trauma.

59. The method of claim 56, wherein the progranulin-associated disorder is Frontotemporal dementia (FTD).

60. The method of claim 56, wherein the progranulin-associated disorder is Frontotemporal dementia-Granulin subtype (FTD-GRN).
